(19) United States
(12) Patent Application Publication
(10) Pub. No.: US 2026/0364057 A1
(43) Pub. Date: Nov. 19, 2026

(54) AMIDE COMPOUND, AND COMPOSITION AND USE THEREOF

(71) Applicant: SIMCERE ZAIMING PHARMACEUTICAL CO., LTD., Haikou, Hainan (CN)

(72) Inventors: Jian YU, Shanghai (CN); Yang YANG, Shanghai (CN); Muxing ZHOU, Shanghai (CN); Wei ZHU, Shanghai (CN); Tianwen SUN, Shanghai (CN); Donglin GAO, Shanghai (CN); Feng ZHOU, Shanghai (CN); Lei ZHOU, Shanghai (CN); Wansun MAI, Shanghai (CN); Zhengtao LI, Shanghai (CN)

(21) Appl. No.: 19/109,919

(22) PCT Filed: Sep. 8, 2023
(86) PCT No.: PCT/CN2023/117694

§ 371 Date: Mar. 7, 2025

(30) Foreign Application Priority Data
Sep. 9, 2022 (CN) 202211103512.1
Nov. 24, 2022 (CN) 202211481864.0
Apr. 13, 2023 (CN) 202310395648.2
Jul. 28, 2023 (CN) 202310951733.2

(51) Int. Cl. A61K 31/438; A61K 31/439; A61K 31/501; A61K 31/513; A61K 31/519; A61K 31/5377; A61K 31/5383; A61K 31/5386; A61K 31/55; A61K 31/553; A61P 35/00; C07D 401/12; C07D 401/14; C07D 405/12; C07D 471/04; C07D 471/14; C07D 471/18; C07D 487/04; C07D 498/04; C07D 498/08; C07D 498/14; C07D 513/04

(52) U.S. Cl. CPC A61K 31/438; A61K 31/439; A61K 31/501; A61K 31/513; A61K 31/519; A61K 31/5377; A61K 31/5383; A61K 31/5386; A61K 31/55; A61K 31/553; A61P 35/00; C07D 401/12; C07D 401/14; C07D 405/12; C07D 471/04; C07D 471/14; C07D 471/18; C07D 487/04; C07D 498/04; C07D 498/08; C07D 498/14; C07D 513/04

(57) ABSTRACT

The present invention relates to a novel amide compound or a pharmaceutically acceptable salt thereof, a pharmaceutical composition containing same, and a use thereof as a KIF18A inhibitor in prevention or treatment of related diseases.

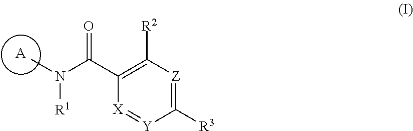

### CROSS-REFERENCE TO RELATED APPLICATION

The present application claims the benefit of and priority to the following four Chinese Patent Applications for Invention, the content of which is incorporated herein by reference in their entirety:
Chinese Patent Application No. 202211103512.1 filed with China National Intellectual Property Administration on Sep. 9, 2022;
Chinese Patent Application No. 202211481864.0 filed with China National Intellectual Property Administration on Nov. 24, 2022;
Chinese Patent Application No. 202310395648.2 filed with China National Intellectual Property Administration on Apr. 13, 2023; and
Chinese Patent Application No. 202310951733.2 filed with China National Intellectual Property Administration on Jul. 28, 2023.

### TECHNICAL FIELD

The present disclosure relates to a novel amide compound or a pharmaceutically acceptable salt thereof, a pharmaceutical composition containing same, and use thereof as a KIF18A inhibitor in the prevention or treatment of related diseases.

### BACKGROUND

Cancer is generally characterized by unregulated cell proliferation. Damage to one or more genes of the cell proliferation pathway may lead to the loss of normal regulation of cell proliferation. These deregulated genes can encode various tumor suppressors or oncogene proteins, leading to unchecked cell cycle progression and cell proliferation. Various kinases and kinesins have been identified as key regulators of cell cycle, mitotic regulation, and progression of normal cells and cancer cells.

Kinesins are molecular motors that play important roles in cell division and intracellular transport of vesicles and organelles. Mitotic kinesins play a role in spindle assembly, chromosome segregation, centrosome segregation, and many other aspects. Based on sequence homology within the so-called “motor domain”, human kinesins are classified into 14 subfamilies. The activity of ATPase of the domain enables unidirectional movement along microtubules. The non-motor domains of these proteins are responsible for attachment to the “cargo”; the “cargo” may be one of a variety of membranous organelles, signal transduction scaffold systems, and chromosomes. Kinesins use the energy resulting from ATP hydrolysis to move the “cargo” along polarized microtubules. Therefore, kinesins are often referred to as “plus-end-directed” or “minus-end-directed” motor proteins. The KIF18A gene belongs to the kinesin-8 subfamily and is a plus-end-directed motor protein. KIF18A is believed to affect the movement of the plus end of the kinetochore microtubules to control proper chromosome positioning and maintain spindle tension. Removal of human KIF18A results in longer spindles in HeLa cervical cancer cells, increases chromosome oscillation in the metaphase of mitosis and results in activation of the spindle assembly checkpoint. Thus, KIF18A may be a viable target for cancer therapy, with KIF18A being overexpressed in various types of cancer. In addition, in cancer cells, the deletion, knockout, or inhibition of the KIF18A gene affects the mitotic spindle. Specifically, it has been found that inhibiting KIF18A induces mitotic arrest, ultimately leading to mitotic catastrophe of cancer cells or mitotic slippage in interphase and thereby resulting in apoptosis of cancer cells.

Therefore, inhibitors of KIF18A can be developed into promising anticancer drugs, and research institutions are highly interested in discovering inhibitors of the KIF18A protein.

### SUMMARY

The present disclosure provides a compound of formula (I) or a pharmaceutically acceptable salt thereof:

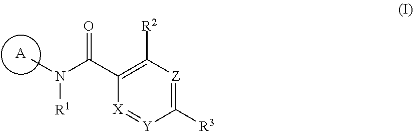

wherein
ring A is selected from 5-membered heteroaryl, pyridinonyl, azapyridinonyl,

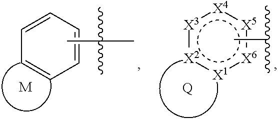

10-16 membered tricyclic heterocyclyl, 12-20 membered tetracyclic heterocyclyl, and 11-14 membered tricyclic heteroaryl;
the 5-membered heteroaryl is optionally substituted with one or more R^{1a}, and each R^{1a} is independently selected from halogen, CN, OH, NH₂, C₁-C₁₀ alkyl, C₂-C₁₀ alkenyl, C₂-C₁₀ alkynyl, C₃-C₁₀ cycloalkyl, C₅-C₁₀ cycloalkenyl, 4-10 membered heterocyclyl, C₆-C₁₀ aryl, and 5-10 membered heteroaryl, wherein the OH, NH₂, C₁-C₁₀ alkyl, C₂-C₁₀ alkenyl, C₂-C₁₀ alkynyl, C₃-C₁₀ cycloalkyl, C₅-C₁₀ cycloalkenyl, 4-10 membered heterocyclyl, C₆-C₁₀ aryl, or 5-10 membered heteroaryl is optionally substituted with one or more R^{1b}; when two R^{1a} are located at the ortho position of the 5-membered heteroaryl, the two R^{1a}, together with the atom to which they are attached, can also form C₅-C₇ monocyclic alkenyl, 4-7 membered monoheterocyclyl, phenyl, or 5-6 membered monoheteroaryl, wherein the C₅-C₇ monocyclic alkenyl, 4-7 membered monoheterocyclyl, phenyl, or 5-6 membered monoheteroaryl is optionally substituted with one or more R^{1b};
the pyridinonyl, azapyridinonyl, 10-16 membered tricyclic heterocyclyl, 12-20 membered tetracyclic heterocyclyl, or 11-14 membered tricyclic heteroaryl is optionally substituted with one or more R^{2b};
the ring M is selected from C₄-C₇ monocyclic alkenyl, 4-7 membered monoheterocyclyl, phenyl, and 5-6 membered monoheteroaryl; the structural unit

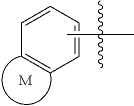

is substituted with one or more R^{3a}, wherein the R^{3a} is selected from C₃-C₁₀ cycloalkyl, C₅-C₁₀ cycloalkenyl, 4-10 membered heterocyclyl, C₆-C₁₀ aryl, and 5-10 membered heteroaryl; optionally, the

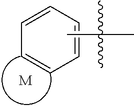

substituted with one or more R^{3a} can be further substituted with one or more R^{3b};

1, 2 or 3 of the X¹, X², X³, X⁴, X⁵, and X⁶ are selected from N and the others are selected from CR⁴, or 1 of the X³, X⁴, X⁵, and X⁶ is absent and the other 3, together with X¹ and X², form 5-membered heteroaryl, wherein the 5-membered heteroaryl is optionally substituted with one or more R^{4b};

each R⁴ is independently selected from being absent, H, and R^{4b};

the ring Q is selected from C₅-C₇ monocyclic alkenyl, 4-7 membered monoheterocyclyl, phenyl, and 5-6 membered monoheteroaryl, wherein the C₅-C₇ monocyclic alkenyl, 4-7 membered monoheterocyclyl, phenyl, or 5-6 membered monoheteroaryl is optionally substituted with one or more R^{4b};

R¹ is selected from H and C₁-C₆ alkyl, wherein the C₁-C₆ alkyl is optionally substituted with one or more halogen;
R² is selected from

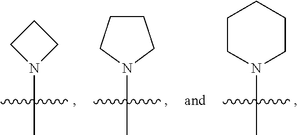

and the

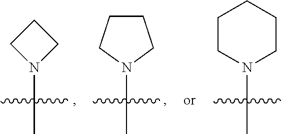

is optionally substituted with one or more R^{x}, wherein each R^{x} is independently selected from halogen, CN, OH, NH₂, C₁-C₆ alkyl, C₃-C₆ cycloalkyl, and 4-7 membered heterocyclyl, wherein the OH, NH₂, C₁-C₆ alkyl, C₃-C₆ cycloalkyl, or 4-7 membered heterocyclyl is optionally substituted with one or more R^{xb}; when the same carbon atom on

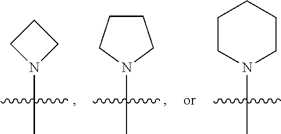

is simultaneously substituted with two R^{x}, the two R^{x}, together with the carbon atom, can also form C₃-C₁₀ cycloalkyl, C₃-C₁₀ cycloalkenyl, or 4-10 membered heterocyclyl, wherein the C₃-C₁₀ cycloalkyl, C₃-C₁₀ cycloalkenyl, or 4-10 membered heterocyclyl is optionally substituted with one or more R^{xb};

R³ is selected from L¹-L²-R^{y}, wherein L¹ is selected from —NR⁵—, —O—, —S—, —S(═O)—, —SO₂—, —C(═O)—, —NR⁵SO₂—, —NRSC(═O)—, —NR⁵—S(═O)(═NR⁵)—, —S(═O)(═NR⁵)—, —S═N—,

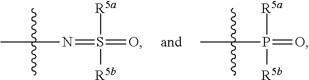

each R⁵ is independently selected from H and C₁-C₄ alkyl, each R^{5a} and R^{5b} is independently selected from C₁-C₄ alkyl, C₃-C₆ cycloalkyl, and 4-7 membered heterocyclyl, or the R^{5a} and R^{5b}, together with the atom to which they are attached, form 4-7 membered heterocyclyl; L² is selected from a chemical bond and C₁-C₄ alkylene; R^{y} is selected from H, C₃-C₆ cycloalkyl, and 4-7 membered heterocyclyl; each C₁-C₄ alkyl, C₁-C₄ alkylene, C₃-C₆ cycloalkyl, or 4-7 membered heterocyclyl described above is optionally substituted with one or more halogen, CN, OH, NH₂, O(C₁-C₄ alkyl), NH(C₁-C₄ alkyl), or N(C₁-C₄ alkyl)₂;
X, Y, and Z are each independently selected from N and CR⁶, wherein the R⁶ is selected from H, halogen, CN, OH, NH₂, C₁-C₆ alkyl, C₃-C₆ cycloalkyl, 4-7 membered heterocyclyl, O(C₁-C₄ alkyl), NH(C₁-C₄ alkyl), and N(C₁-C₄ alkyl)₂, wherein the C₁-C₆ alkyl, C₃-C₆ cycloalkyl, 4-7 membered heterocyclyl, O(C₁-C₄ alkyl), NH(C₁-C₄ alkyl), or N(C₁-C₄ alkyl)₂ is optionally substituted with one or more halogen or CN;
each R^{1b}, R^{2b}, R^{3b}, R^{4b}, and R^{xb} is independently selected from halogen, CN, OH, NH₂, C₁-C₆ alkyl, C₂-C₆ alkenyl, C₅-C₁₀ cycloalkenyl, C(O)R^{d}, C₃-C₆ cycloalkyl, 4-14 membered heterocyclyl, O(C₁-C₄ alkyl), NH(C₁-C₄ alkyl), and N(C₁-C₄ alkyl)₂, wherein the C₁-C₆ alkyl, C₂-C₆ alkenyl, C₅-C₁₀ cycloalkenyl, C₃-C₆ cycloalkyl, 4-14 membered heterocyclyl, O(C₁-C₄ alkyl), NH(C₁-C₄ alkyl), or N(C₁-C₄ alkyl)₂ is optionally substituted with one or more R^{c};
each R^{c} is independently selected from halogen, CN, OH, NH₂, C₁-C₆ alkyl, C₃-C₆ cycloalkyl, 4-7 membered heterocyclyl, O(C₁-C₄ alkyl), NH(C₁-C₄ alkyl), and N(C₁-C₄ alkyl)₂;
R^{d} is selected from OH, C₁-C₆ alkyl, O(C₁-C₄ alkyl), NH₂, NH(C₁-C₄ alkyl), and N(C₁-C₄ alkyl)₂.

In some embodiments, ring A is selected from 5-membered heteroaryl, pyridinonyl, azapyridinonyl,

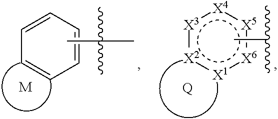

10-16 membered tricyclic heterocyclyl, 12-20 membered tetracyclic heterocyclyl, and 11-14 membered tricyclic heteroaryl;
the 5-membered heteroaryl is optionally substituted with one or more R^{1a}, and each R^{1a} is independently selected from halogen, CN, OH, NH₂, C₁-C₁₀ alkyl, C₂-C₁₀ alkenyl, C₂-C₁₀ alkynyl, C₃-C₁₀ cycloalkyl, C₅-C₁₀ cycloalkenyl, 4-10 membered heterocyclyl, C₆-C₁₀ aryl, and 5-10 membered heteroaryl, wherein the OH, NH₂, C₁-C₁₀ alkyl, C₂-C₁₀ alkenyl, C₂-C₁₀ alkynyl, C₃-C₁₀ cycloalkyl, C₅-C₁₀ cycloalkenyl, 4-10 membered heterocyclyl, C₆-C₁₀ aryl, or 5-10 membered heteroaryl is optionally substituted with one or more R^{1b}; when two R^{1a} are located at the ortho position of the 5-membered heteroaryl, the two Ria, together with the atom to which they are attached, can also form C₅-C₇ monocyclic alkenyl, 4-7 membered monoheterocyclyl, phenyl, or 5-6 membered monoheteroaryl, wherein the C₅-C₇ monocyclic alkenyl, 4-7 membered monoheterocyclyl, phenyl, or 5-6 membered monoheteroaryl is optionally substituted with one or more R^{1b};
the pyridinonyl, azapyridinonyl, 10-16 membered tricyclic heterocyclyl, 12-20 membered tetracyclic heterocyclyl, or 11-14 membered tricyclic heteroaryl is optionally substituted with one or more R^{2b};
the ring M is selected from C₅-C₇ monocyclic alkenyl, 4-7 membered monoheterocyclyl, phenyl, and 5-6 membered monoheteroaryl; the structural unit

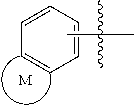

is substituted with one or more R^{3a}, wherein the R^{3a} is selected from C₃-C₁₀ cycloalkyl, C₅-C₁₀ cycloalkenyl, 4-10 membered heterocyclyl, C₆-C₁₀ aryl, and 5-10 membered heteroaryl; optionally, the

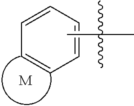

substituted with one or more R^{3a} can be further substituted with one or more R^{3b};
1, 2 or 3 of the X¹, X², X³, X⁴, X⁵, and X⁶ are selected from N and the others are selected from CR⁴, or 1 of the X³, X⁴, X⁵, and X⁶ is absent and the other 3, together with X¹ and X², form 5-membered heteroaryl, wherein the 5-membered heteroaryl is optionally substituted with one or more R^{4b};
each R⁴ is independently selected from being absent, H, and R^{4b};
the ring Q is selected from C₅-C₇ monocyclic alkenyl, 4-7 membered monoheterocyclyl, phenyl, and 5-6 membered monoheteroaryl, wherein the C₅-C₇ monocyclic alkenyl, 4-7 membered monoheterocyclyl, phenyl, or 5-6 membered monoheteroaryl is optionally substituted with one or more R^{4b};
R¹ is selected from H and C₁-C₆ alkyl, wherein the C₁-C₆ alkyl is optionally substituted with one or more halogen;
R² is selected from

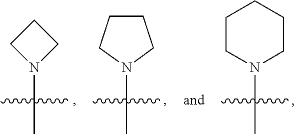

and the

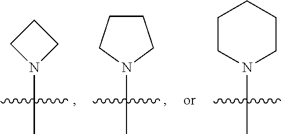

is optionally substituted with one or more R^{x}, wherein each R^{x} is independently selected from halogen, CN, OH, NH₂, C₁-C₆ alkyl, C₃-C₆ cycloalkyl, and 4-7 membered heterocyclyl, wherein the OH, NH₂, C₁-C₆ alkyl, C₃-C₆ cycloalkyl, or 4-7 membered heterocyclyl is optionally substituted with one or more R^{xb}; when the same carbon atom on

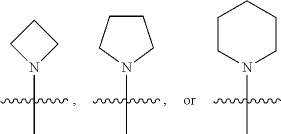

is simultaneously substituted with two R^{x}, the two R^{x}, together with the carbon atom, can also form C₃-C₁₀ cycloalkyl, C₃-C₁₀ cycloalkenyl, or 4-10 membered heterocyclyl, wherein the C₃-C₁₀ cycloalkyl, C₃-C₁₀ cycloalkenyl, or 4-10 membered heterocyclyl is optionally substituted with one or more R^{xb};
R³ is selected from L¹-L²-R^{y}, wherein Li is selected from —NR⁵—, —O—, —S—, —S(═O)—, —SO₂—, —C(═O)—, —NR⁵SO₂—, —NR⁵C(═O)—, —NR⁵—S(═O)(═NR⁵)—, —S(═O)(═NR⁵)—, —S═N—,

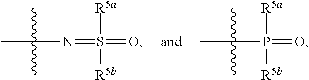

each R⁵ is independently selected from H and C₁-C₄ alkyl, each R^{5a} and R^{5b} is independently selected from C₁-C₄ alkyl, C₃-C₆ cycloalkyl, and 4-7 membered heterocyclyl, or the R^{5a} and R^{5b}, together with the atom to which they are attached, form 4-7 membered heterocyclyl; L² is selected from a chemical bond and C₁-C₄ alkylene; R^{y} is selected from H, C₃-C₆ cycloalkyl, and 4-7 membered heterocyclyl; each C₁-C₄ alkyl, C₁-C₄ alkylene, C₃-C₆ cycloalkyl, or 4-7 membered heterocyclyl described above is optionally substituted with one or more halogen, CN, OH, NH₂, O(C₁-C₄ alkyl), NH(C₁-C₄ alkyl), or N(C₁-C₄ alkyl)₂;
X, Y, and Z are each independently selected from N and CR⁶, wherein the R⁶ is selected from H, halogen, CN, OH, NH₂, C₁-C₆ alkyl, C₃-C₆ cycloalkyl, 4-7 membered heterocyclyl, O(C₁-C₄ alkyl), NH(C₁-C₄ alkyl), and N(C₁-C₄ alkyl)₂, wherein the C₁-C₆ alkyl, C₃-C₆ cycloalkyl, 4-7 membered heterocyclyl, O(C₁-C₄ alkyl), NH(C₁-C₄ alkyl), or N(C₁-C₄ alkyl)₂ is optionally substituted with one or more halogen or CN;
each R^{1b}, R^{2b}, R^{3b}, R^{4b}, and R^{xb} is independently selected from halogen, CN, OH, NH₂, C₁-C₆ alkyl, C₂-C₆ alkenyl, C₃-C₆ cycloalkyl, 4-14 membered heterocyclyl, O(C₁-C₄ alkyl), NH(C₁-C₄ alkyl), and N(C₁-C₄ alkyl)₂, wherein the C₁-C₆ alkyl, C₂-C₆ alkenyl, C₃-C₆ cycloalkyl, 4-14 membered heterocyclyl, O(C₁-C₄ alkyl), NH(C₁-C₄ alkyl), or N(C₁-C₄ alkyl)₂ is optionally substituted with one or more R^{c};
each R^{c} is independently selected from halogen, CN, OH, NH₂, C₁-C₆ alkyl, C₃-C₆ cycloalkyl, 4-7 membered heterocyclyl, O(C₁-C₄ alkyl), NH(C₁-C₄ alkyl), and N(C₁-C₄ alkyl)₂.

In some embodiments, 1, 2, or 3 of the X¹, X², X³, X⁴, X⁵, and X⁶ are selected from N, and the others are selected from CR⁴, wherein each R⁴ is each independently selected from being absent, H, and R^{4b}; each R^{1b}, R^{2b}, R^{3b}, R^{4b}, and R^{xb} is independently selected from halogen, CN, OH, NH₂, C₁-C₆ alkyl, C₃-C₆ cycloalkyl, 4-14 membered heterocyclyl, O(C₁-C₄ alkyl), NH(C₁-C₄ alkyl), and N(C₁-C₄ alkyl)₂, wherein the C₁-C₆ alkyl, C₃-C₆ cycloalkyl, 4-14 membered heterocyclyl, O(C₁-C₄ alkyl), NH(C₁-C₄ alkyl) or N(C₁-C₄ alkyl)₂ is optionally substituted with one or more R^{c};
each R^{c} is independently selected from halogen, CN, OH, NH₂, C₁-C₆ alkyl, C₃-C₆ cycloalkyl, 4-7 membered heterocyclyl, O(C₁-C₄ alkyl), NH(C₁-C₄ alkyl), and N(C₁-C₄ alkyl)₂.

In some embodiments, provided is the compound of formula (I) or the pharmaceutically acceptable salt thereof, wherein ring A is selected from 5-membered heteroaryl, pyridinonyl, azapyridinonyl,

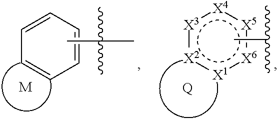

10-16 membered tricyclic heterocyclyl, and 11-14 membered tricyclic heteroaryl;
the 5-membered heteroaryl is optionally substituted with one or more R^{1a}, and each R^{1a} is independently selected from halogen, CN, OH, NH₂, C₁-C₁₀ alkyl, C₂-C₁₀ alkenyl, C₂-C₁₀ alkynyl, C₃-C₁₀ cycloalkyl, C₅-C₁₀ cycloalkenyl, 4-10 membered heterocyclyl, C₆-C₁₀ aryl, and 5-10 membered heteroaryl, wherein the OH, NH₂, C₁-C₁₀ alkyl, C₂-C₁₀ alkenyl, C₂-C₁₀ alkynyl, C₃-C₁₀ cycloalkyl, C₅-C₁₀ cycloalkenyl, 4-10 membered heterocyclyl, C₆-C₁₀ aryl, or 5-10 membered heteroaryl is optionally substituted with one or more R^{1b}; when two R^{1a} are located at the ortho position of the 5-membered heteroaryl, the two R^{1a}, together with the atom to which they are attached, can also form C₅-C₇ monocyclic alkenyl, 4-7 membered monoheterocyclyl, phenyl, or 5-6 membered monoheteroaryl, wherein the C₅-C₇ monocyclic alkenyl, 4-7 membered monoheterocyclyl, phenyl, or 5-6 membered monoheteroaryl is optionally substituted with one or more R^{1b};
the pyridinonyl, azapyridinonyl, 10-16 membered tricyclic heterocyclyl, or 11-14 membered tricyclic heteroaryl is optionally substituted with one or more R^{2b};
the ring M is selected from C₅-C₇ monocyclic alkenyl, 4-7 membered monoheterocyclyl, phenyl, and 5-6 membered monoheteroaryl; the structural unit

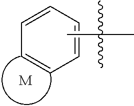

is substituted with one or more R^{3a}, wherein the R^{3a} is selected from C₃-C₁₀ cycloalkyl, C₅-C₁₀ cycloalkenyl, 4-10 membered heterocyclyl, C₆-C₁₀ aryl, and 5-10 membered heteroaryl; optionally, the

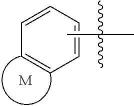

substituted with one or more R^{3a} can be further substituted with one or more R^{3b};
1, 2 or 3 of the X¹, X², X³, X⁴, X⁵, and X⁶ are selected from N, and the others are selected from CR⁴, wherein each R⁴ is independently selected from being absent, H, and R^{4b};
the ring Q is selected from C₅-C₇ monocyclic alkenyl, 4-7 membered monoheterocyclyl, phenyl, and 5-6 membered monoheteroaryl, wherein the C₅-C₇ monocyclic alkenyl, 4-7 membered monoheterocyclyl, phenyl, or 5-6 membered monoheteroaryl is optionally substituted with one or more R^{4b};
R¹ is selected from H and C₁-C₆ alkyl, wherein the C₁-C₆ alkyl is optionally substituted with one or more halogen;
R² is selected from

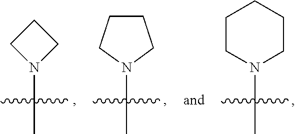

and the

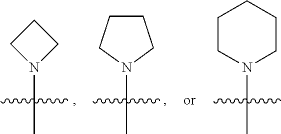

is optionally substituted with one or more R^{x}, wherein each R^{x} is independently selected from halogen, CN, OH, NH₂, C₁-C₆ alkyl, C₃-C₆ cycloalkyl, and 4-7 membered heterocyclyl, wherein the OH, NH₂, C₁-C₆ alkyl, C₃-C₆ cycloalkyl, or 4-7 membered heterocyclyl is optionally substituted with one or more R^{xb}; when the same carbon atom on

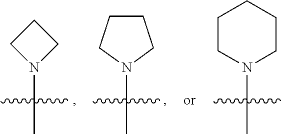

is simultaneously substituted with two R^{x}, the two R^{x}, together with the carbon atom, can also form C₃-C₁₀ cycloalkyl, C₃-C₁₀ cycloalkenyl, or 4-10 membered heterocyclyl, wherein the C₃-C₁₀ cycloalkyl, C₃-C₁₀ cycloalkenyl, or 4-10 membered heterocyclyl is optionally substituted with one or more R^{xb};
R³ is selected from Li-L²-R^{y}, wherein Li is selected from —NR—, —O—, —S—, —S(═O)—, —SO₂—, —(C═O)—, —NR⁵SO₂—, —NR⁵(C═O)—, —NR⁵—S(═O)(═NR⁵)—, and —S(═O)(═NR⁵)—, and each R⁵ is independently selected from H and C₁-C₄ alkyl; L² is selected from a chemical bond and C₁-C₄ alkylene; R^{y} is selected from H, C₃-C₆ cycloalkyl, and 4-7 membered heterocyclyl, wherein the C₁-C₄ alkylene, C₃-C₆ cycloalkyl, or 4-7 membered heterocyclyl is optionally substituted with one or more halogen, CN, OH, NH₂, O(C₁-C₄ alkyl), NH(C₁-C₄ alkyl), or N(C₁-C₄ alkyl)₂;
X, Y, and Z are each independently selected from N and CR⁶, wherein the R⁶ is selected from H, halogen, CN, OH, NH₂, C₁-C₆ alkyl, C₃-C₆ cycloalkyl, 4-7 membered heterocyclyl, O(C₁-C₄ alkyl), NH(C₁-C₄ alkyl), and N(C₁-C₄ alkyl)₂, wherein the C₁-C₆ alkyl, C₃-C₆ cycloalkyl, 4-7 membered heterocyclyl, O(C₁-C₄ alkyl), NH(C₁-C₄ alkyl), or N(C₁-C₄ alkyl)₂ is optionally substituted with one or more halogen or CN;
each R¹¹, R^{2b}, R^{3b}, R^{4b}, and R^{xb} is independently selected from halogen, CN, OH, NH₂, C₁-C₆ alkyl, C₃-C₆ cycloalkyl, 4-7 membered heterocyclyl, O(C₁-C₄ alkyl), NH(C₁-C₄ alkyl), and N(C₁-C₄ alkyl)₂, wherein the C₁-C₆ alkyl, C₃-C₆ cycloalkyl, 4-7 membered heterocyclyl, O(C₁-C₄ alkyl), NH(C₁-C₄ alkyl), or N(C₁-C₄ alkyl)₂ is optionally substituted with one or more R^{c};
each R^{c} is independently selected from halogen, CN, OH, NH₂, C₁-C₆ alkyl, C₃-C₆ cycloalkyl, 4-7 membered heterocyclyl, O(C₁-C₄ alkyl), NH(C₁-C₄ alkyl), and N(C₁-C₄ alkyl)₂.

In some embodiments, ring A is selected from azapyridinonyl,

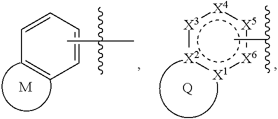

10-16 membered tricyclic heterocyclyl, 12-20 membered tetracyclic heterocyclyl, and 11-14 membered tricyclic heteroaryl, wherein the azapyridinonyl,

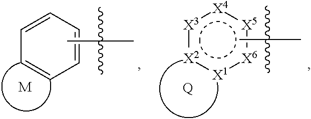

10-16 membered tricyclic heterocyclyl, 12-20 membered tetracyclic heterocyclyl, or 11-14 membered tricyclic heteroaryl is as defined above.

In some embodiments, ring A is selected from azapyridinonyl,

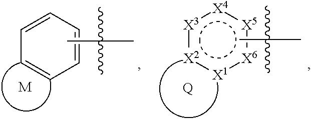

10-16 membered tricyclic heterocyclyl, and 11-14 membered tricyclic heteroaryl, wherein the azapyridinonyl,

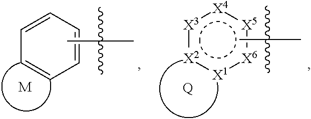

0-16 membered tricyclic heterocyclyl, or 11-14 membered tricyclic heteroaryl is as defined above.

In some embodiments, ring A is selected from azapyridinonyl, 10-16 membered tricyclic heterocyclyl, 12-20 membered tetracyclic heterocyclyl, and 11-14 membered tricyclic heteroaryl, wherein the azapyridinonyl, 10-16 membered tricyclic heterocyclyl, 12-20 membered tetracyclic heterocyclyl, or 11-14 membered tricyclic heteroaryl is selected from the following groups optionally substituted with one or more R^{2b}:

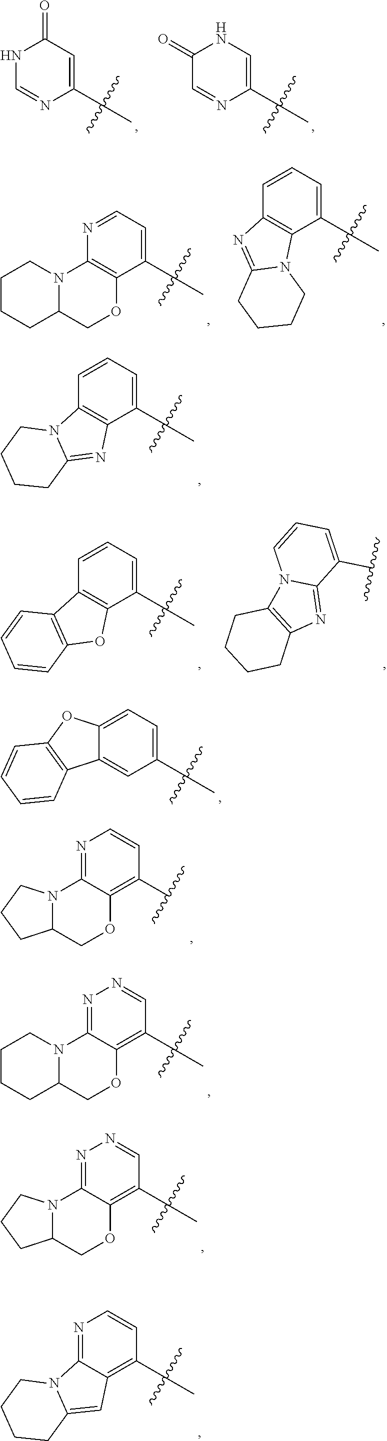

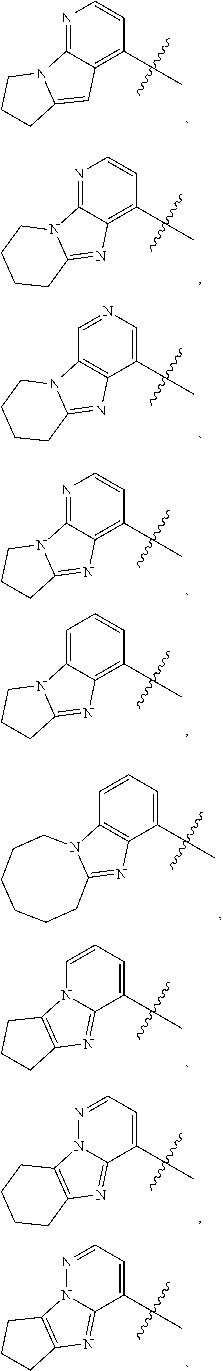

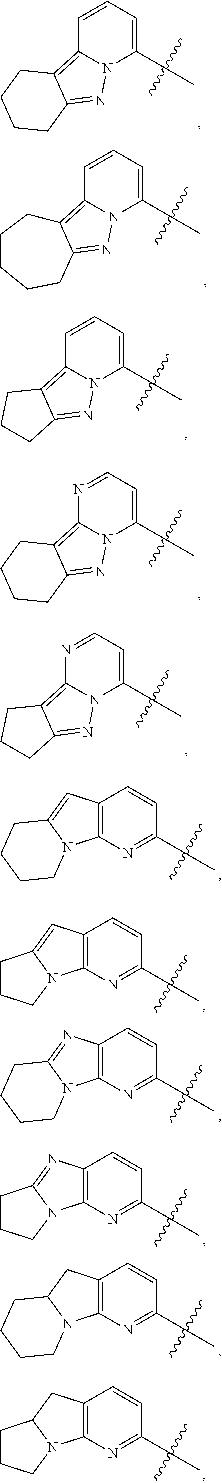

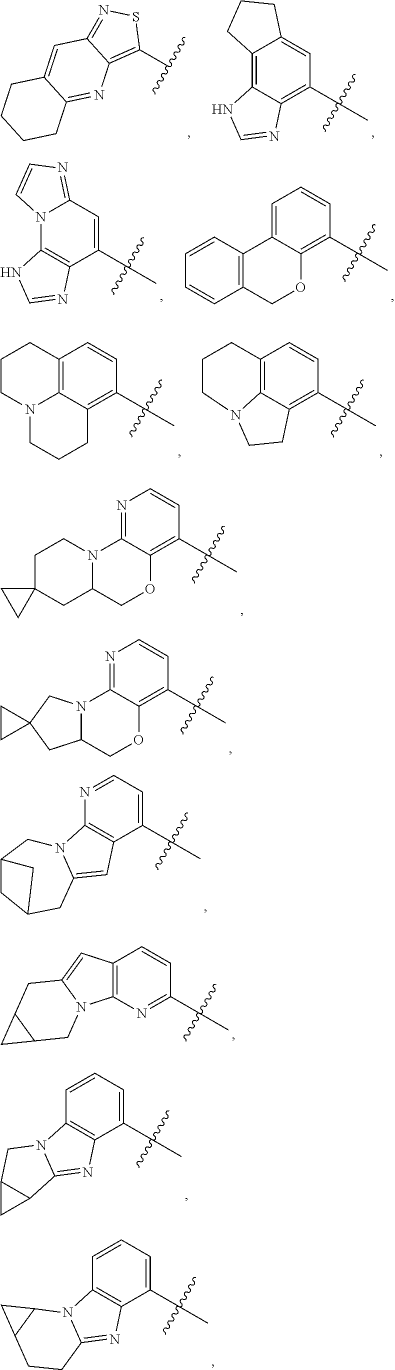

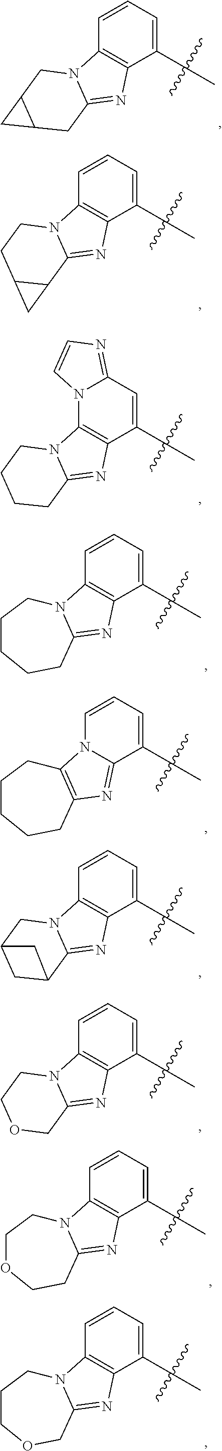

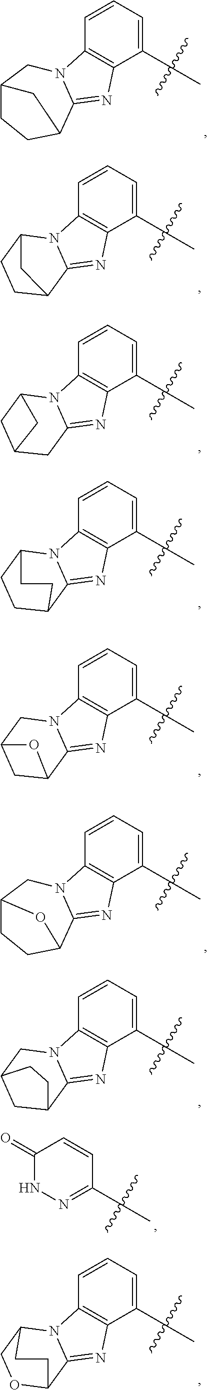

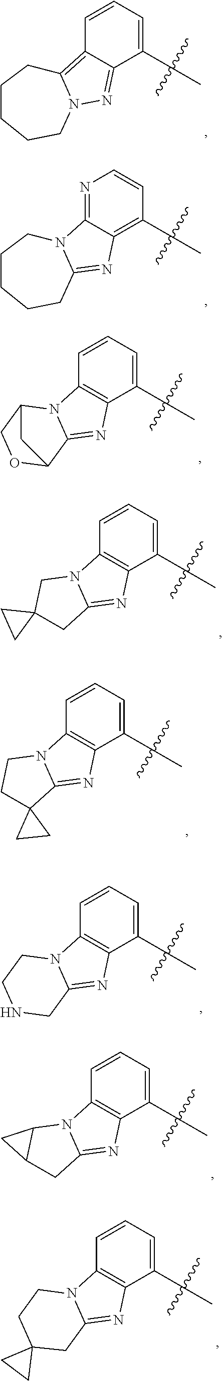

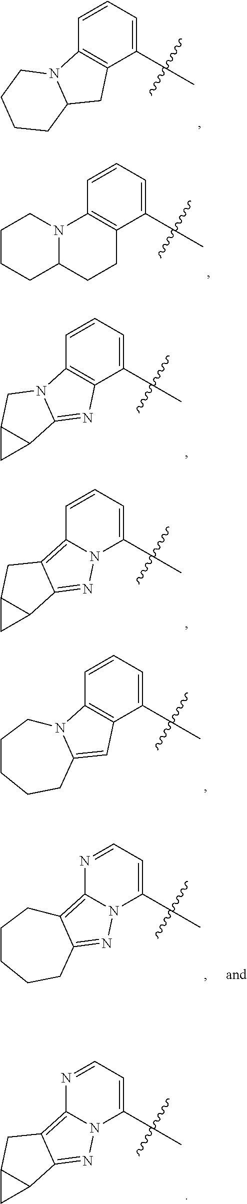

In some embodiments, ring A is selected from azapyridinonyl, 10-16 membered tricyclic heterocyclyl, 12-20 membered tetracyclic heterocyclyl, and 11-14 membered tricyclic heteroaryl, wherein the azapyridinonyl, 10-16 membered tricyclic heterocyclyl, 12-20 membered tetracyclic heterocyclyl, or 11-14 membered tricyclic heteroaryl is selected from the following groups optionally substituted with one or more R^{2b}:

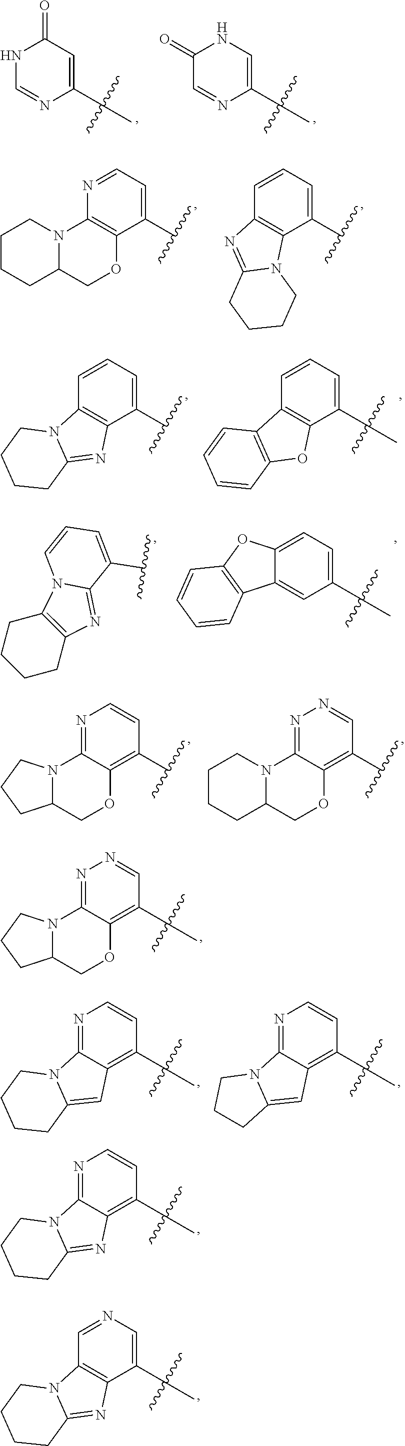

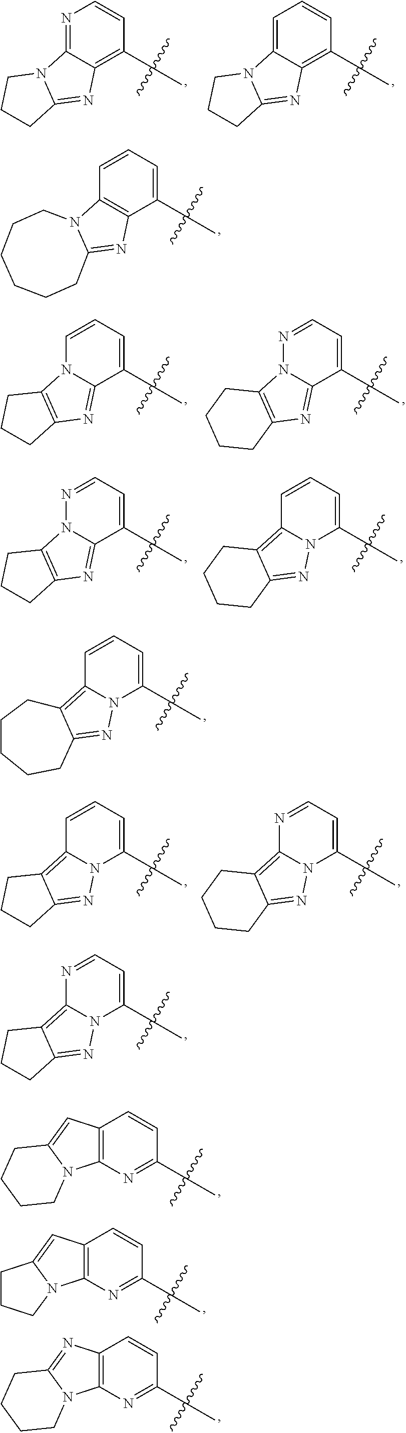

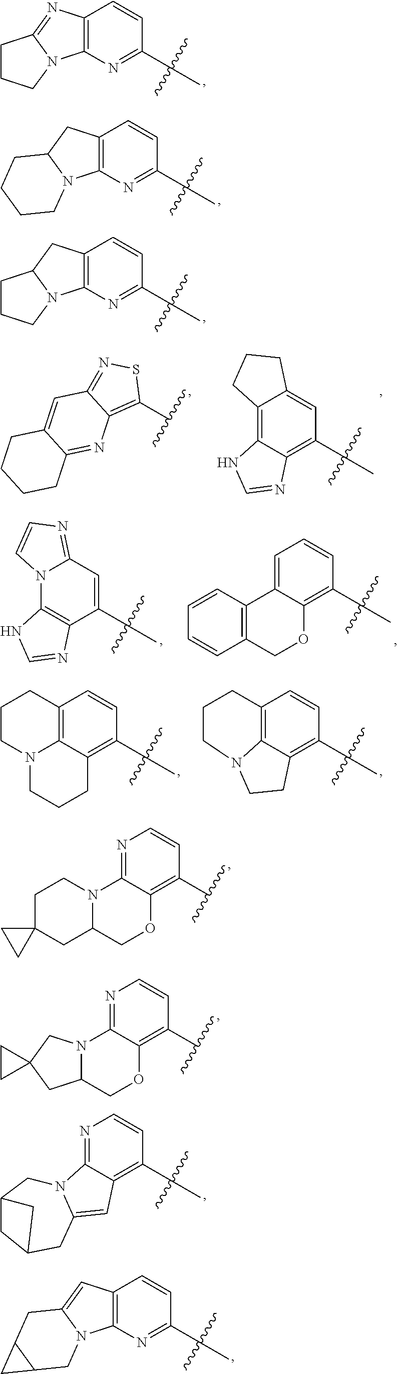

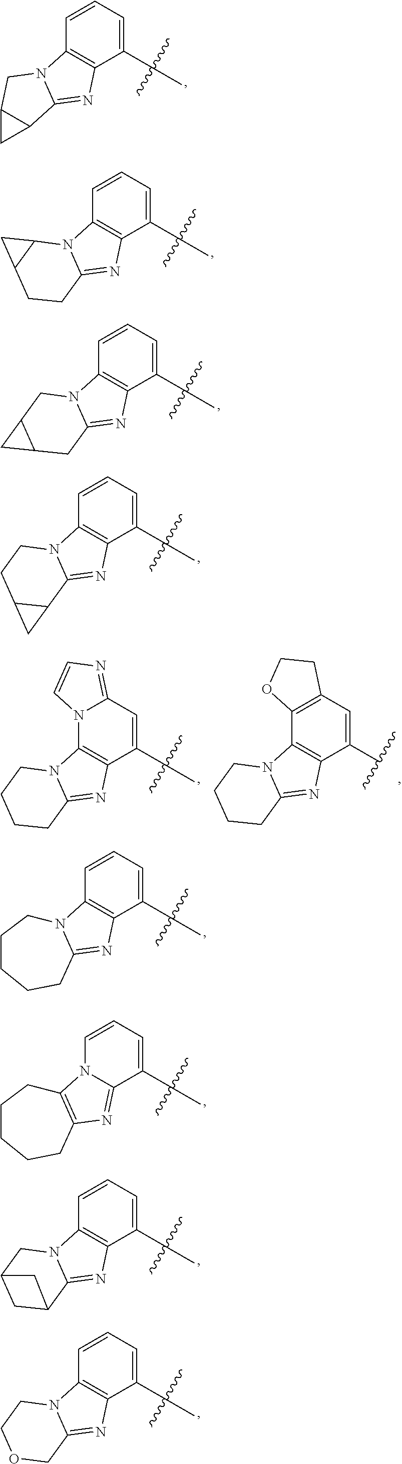

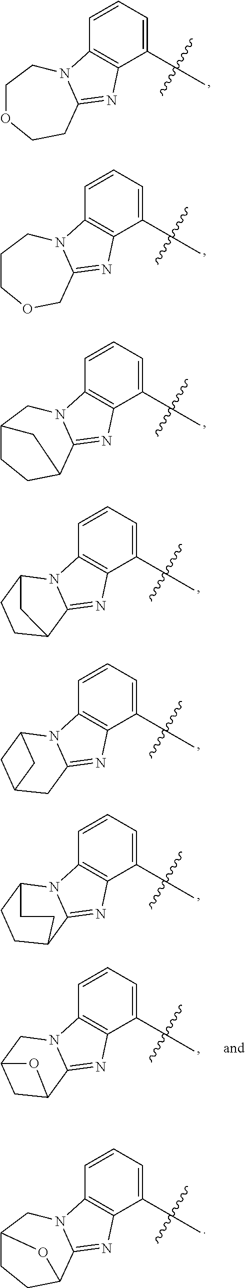

In some embodiments, ring A is selected from azapyridinonyl, 10-16 membered tricyclic heterocyclyl, 12-20 membered tetracyclic heterocyclyl, and 11-14 membered tricyclic heteroaryl, wherein the azapyridinonyl, 10-16 membered tricyclic heterocyclyl, 12-20 membered tetracyclic heterocyclyl, or 11-14 membered tricyclic heteroaryl is selected from the following groups optionally substituted with one or more R^{2b}:

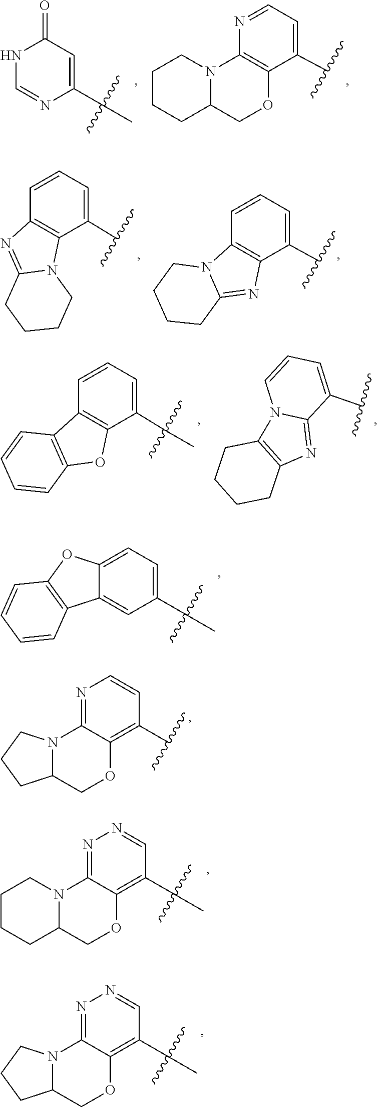

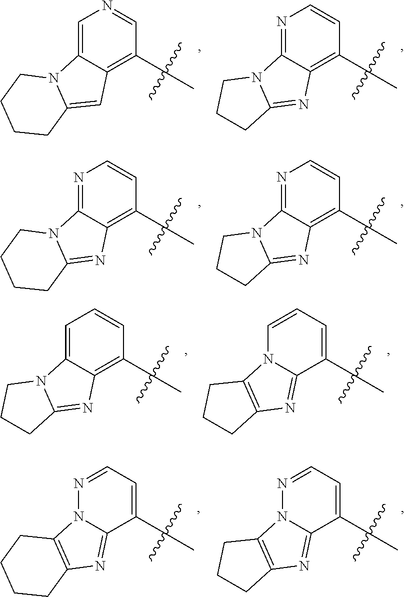

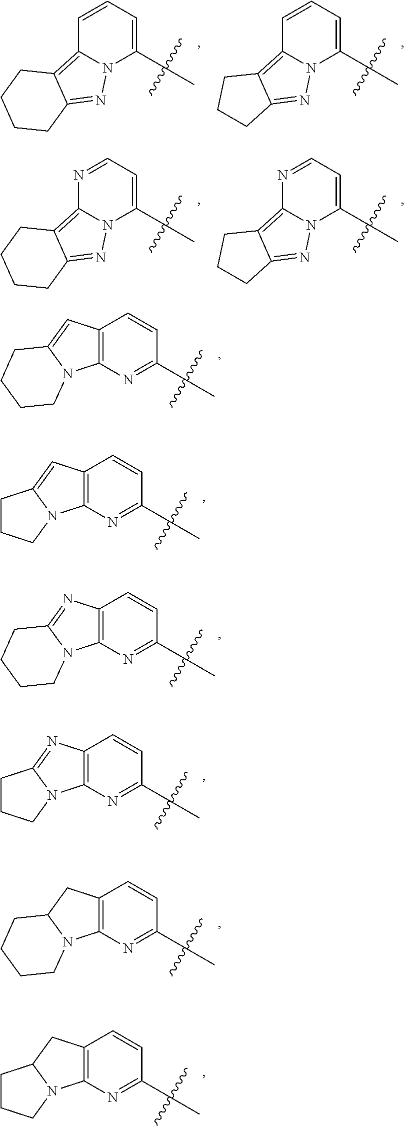

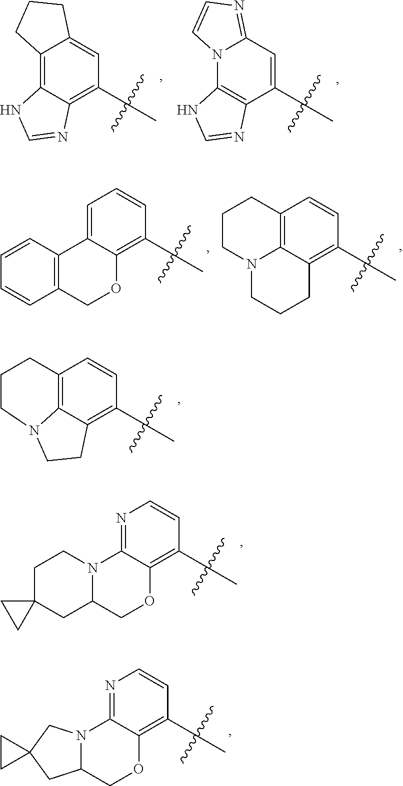

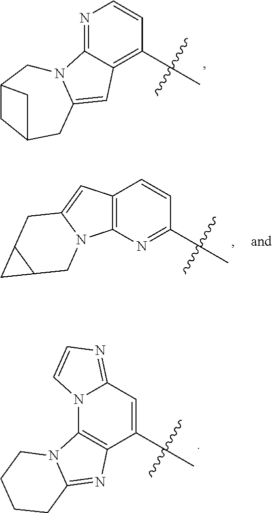 [SMILES: *c1ccnc2c1cc1n2CC2CC(C1)C2 ; *c1ccc2cc3n(c2n1)CC1CC1C3 ; *c1cc2nccn2c2c1nc1n2CCCC1]

In some embodiments, ring A is selected from azapyridinonyl, 10-16 membered tricyclic heterocyclyl, 12-20 membered tetracyclic heterocyclyl, and 11-14 membered tricyclic heteroaryl, wherein the azapyridinonyl, 10-16 membered tricyclic heterocyclyl, 12-20 membered tetracyclic heterocyclyl, or 11-14 membered tricyclic heteroaryl is selected from

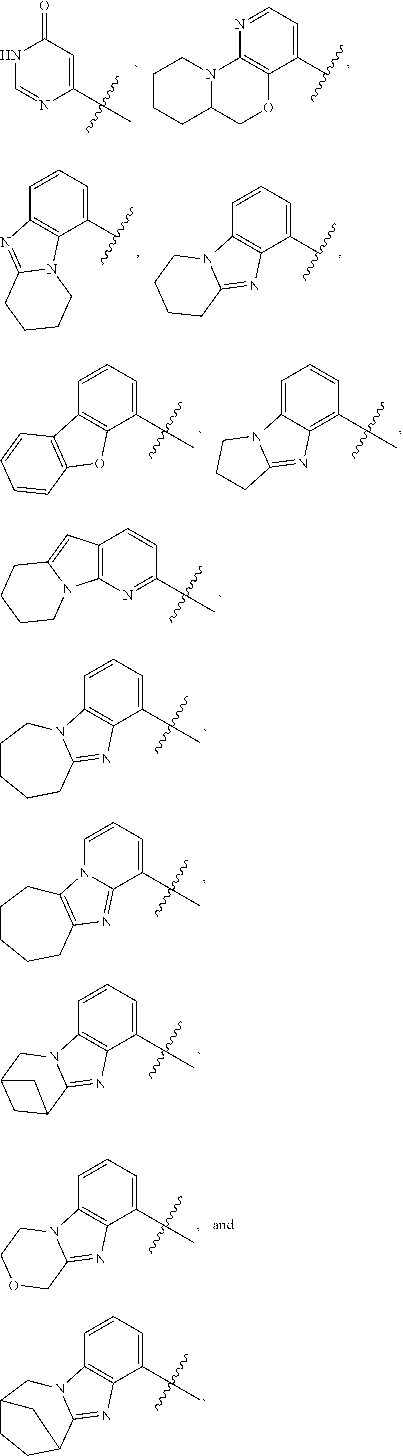

wherein the

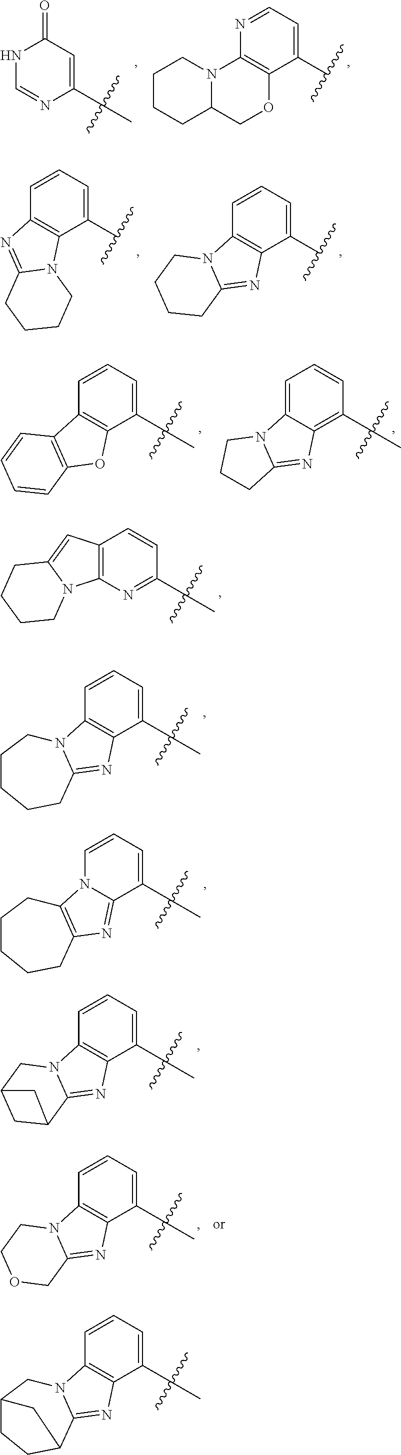

is optionally substituted with one or more R^{2b}.

In some embodiments, the azapyridinonyl, 10-16 membered tricyclic heterocyclyl, or 11-14 membered tricyclic heteroaryl is selected from

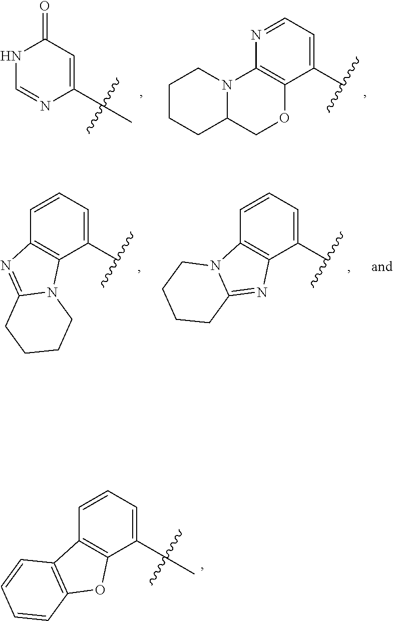 [SMILES: *c1cc(=O)[nH]cn1 ; *c1ccnc2c1OCC1CCCCN21 ; *c1cccc2nc3n(c12)CCCC3 ; *c1cccc2c1nc1n2CCCC1 ; *c1cccc2c1oc1ccccc12]

wherein the

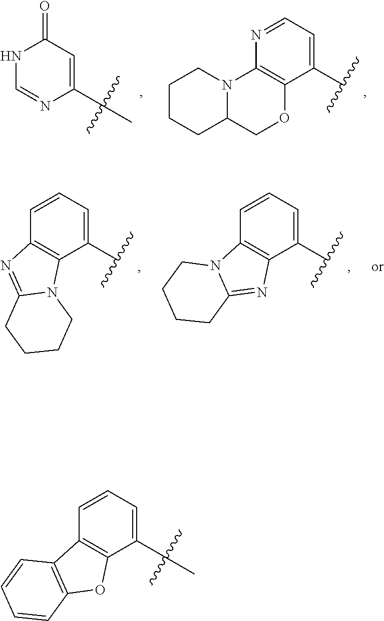 [SMILES: *c1cc(=O)[nH]cn1 ; *c1ccnc2c1OCC1CCCCN21 ; *c1cccc2nc3n(c12)CCCC3 ; *c1cccc2c1nc1n2CCCC1 ; *c1cccc2c1oc1ccccc12]

is optionally substituted with one or more R^{2b}.

In some embodiments, ring M in structural unit

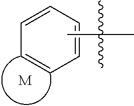

is selected from C₄-C₇ monocyclic alkenyl, 5-6 membered monoheteroaryl, and 4-7 membered monoheterocyclyl, and the structural unit

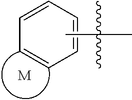

is substituted with one or more R^{3a}; optionally, the structural unit

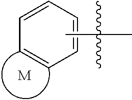

substituted with one or more R^{3a} can be further substituted with one or more R^{3b}.

In some embodiments, the ring M in structural unit

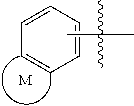

is selected from 5-6 membered monoheteroaryl and 4-7 membered monoheterocyclyl, and the structural unit

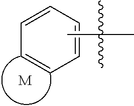

is substituted with one or more R^{3a}; optionally, the structural unit

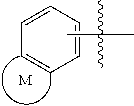

substituted with one or more R^{3a} can be further substituted with one or more R^{3b}.

In some embodiments, the ring M in structural unit

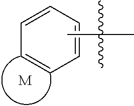

is selected from 5-6 membered monoheteroaryl, and the structural unit

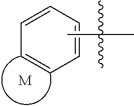

is substituted with one or more R^{3a}; optionally, the structural unit

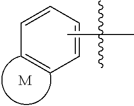

substituted with one or more R^{3a} can be further substituted with one or more R^{3b}.

In some embodiments, the structural unit

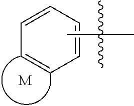

is selected from the following groups substituted with one or more R^{3a}:

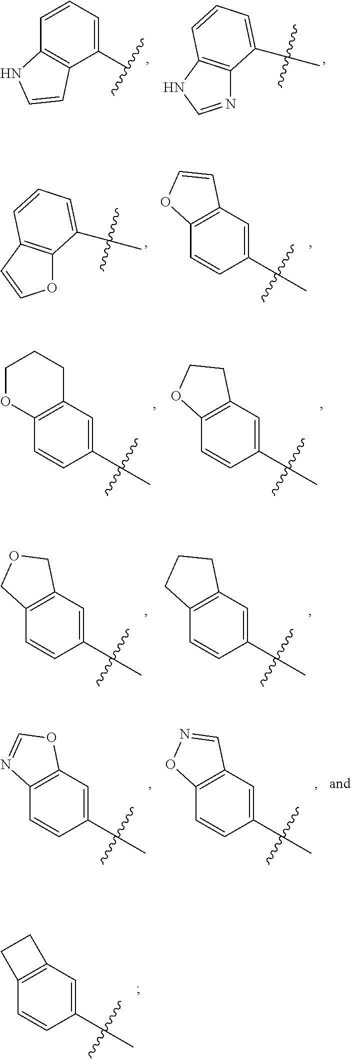

optionally, the aforementioned groups substituted with one or more R^{3a} can be further substituted with one or more R^{3b}.

In some embodiments, the structural unit

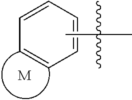

is selected from the following groups substituted with one or more R^{3a}:

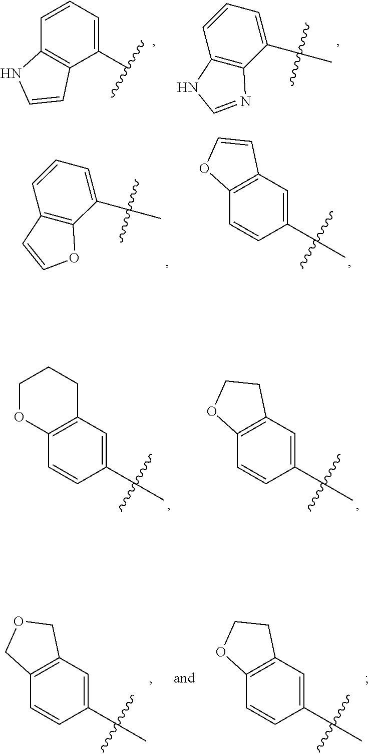

optionally, the aforementioned groups substituted with one or more R^{3a} can be further substituted with one or more R^{3b}.

In some embodiments, the structural unit

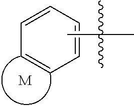

is selected from the following groups substituted with one or more R^{3a}:

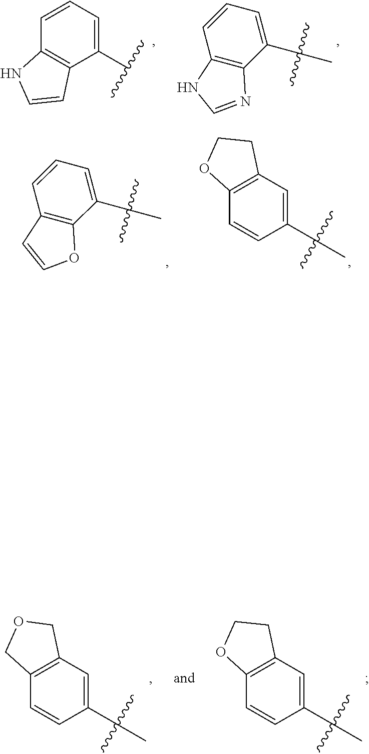

optionally, the aforementioned groups substituted with one or more R^{3a} can be further substituted with one or more R^{3b}.

In some embodiments, the structural unit

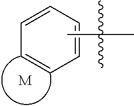

is selected from

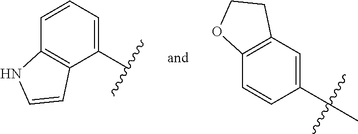 [SMILES: *c1cccc2[nH]ccc12 ; *c1ccc2c(c1)CCO2]

substituted with one or more R^{3a}; optionally, the

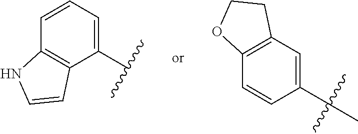 [SMILES: *c1cccc2[nH]ccc12 ; *c1ccc2c(c1)CCO2]

substituted with one or more R^{3a} can be further substituted with one or more R^{3b}.

In some embodiments, the structural unit

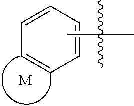

is selected from

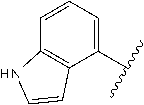 [SMILES: *c1cccc2[nH]ccc12]

substituted with one or more R^{3a}; optionally, the

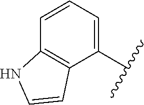 [SMILES: *c1cccc2[nH]ccc12]

substituted with one or more R^{3a} can be further substituted with one or more R^{3b}.

In some embodiments, the structural unit

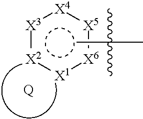

is selected from

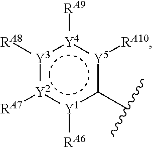

wherein 1, 2, or 3 of ring atoms Y¹, Y², Y³, Y⁴, and Y⁵ are N, and the others are C; any one of R^{A6} and R^{A8}, and R^{A7}, together with the atom to which they are attached, form C₅-C₇ monocyclic alkenyl, 4-7 membered monoheterocyclyl, phenyl, or 5-6 membered monoheteroaryl, wherein the C₅-C₇ monocyclic alkenyl, 4-7 membered monoheterocyclyl, phenyl, or 5-6 membered monoheteroaryl is optionally substituted with one or more R^{4b}; R^{A6}, R^{A8}, R^{A9}, and R^{A10} not involved in the ring forming are each independently selected from being absent, H, and R^{4b}.

In some embodiments, the structural unit

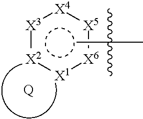

is selected from

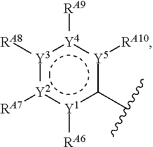

wherein 1 or 2 of ring atoms Y¹, Y², Y³, Y⁴, and Y⁵ of the

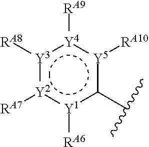

are N, and the others are C; any one of R^{A6} and R^{A8}, and R^{A7}, together with the atom to which they are attached, form C₅ monocyclic alkenyl or 5-6 membered monoheteroaryl, wherein the C₅ monocyclic alkenyl or 5-6 membered monoheteroaryl is optionally substituted with one or more R^{4b}; R^{A6}, R^{A8}, R^{A9}, and R^{A10} not involved in the ring forming are each independently selected from being absent, H, and R^{4b}.

In some embodiments, the structural unit

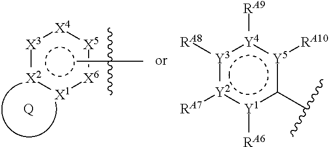

is selected from the following groups optionally substituted with one or more R^{4b}:

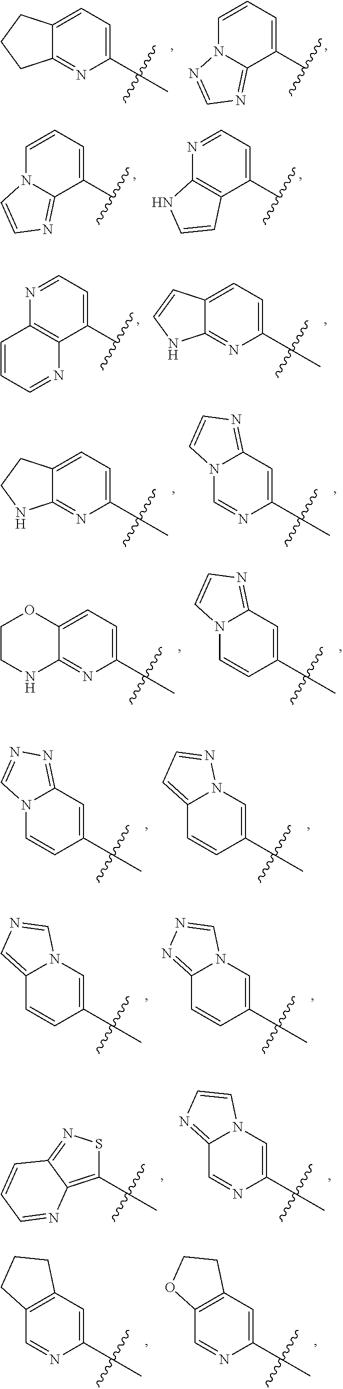

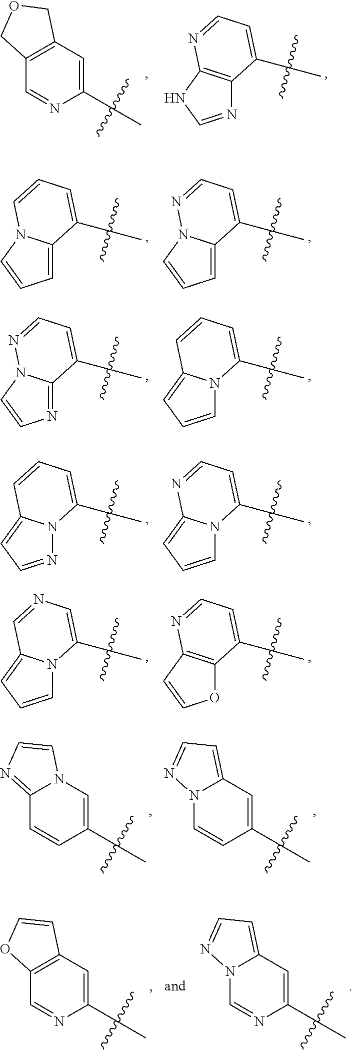

In some embodiments, the structural unit

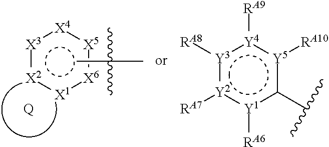

is selected from the following groups optionally substituted with one or more R^{4b}:

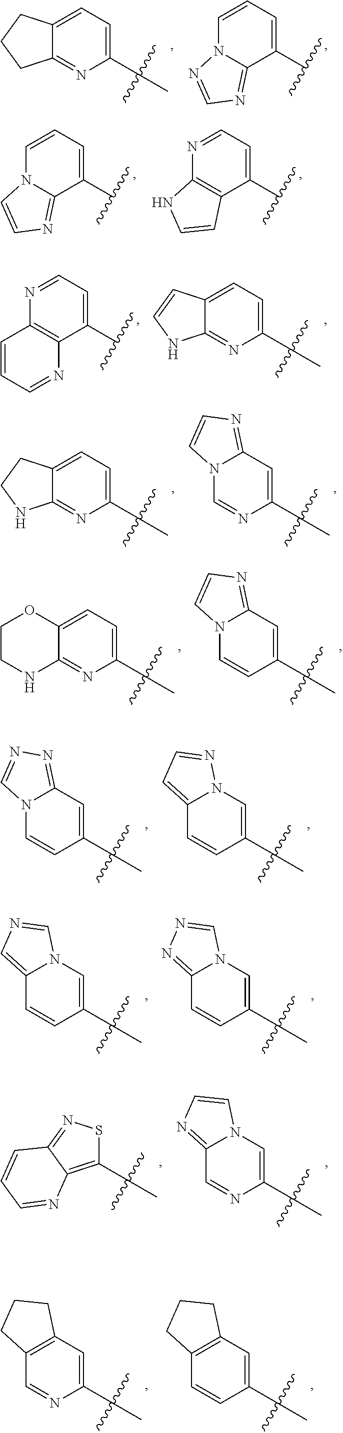

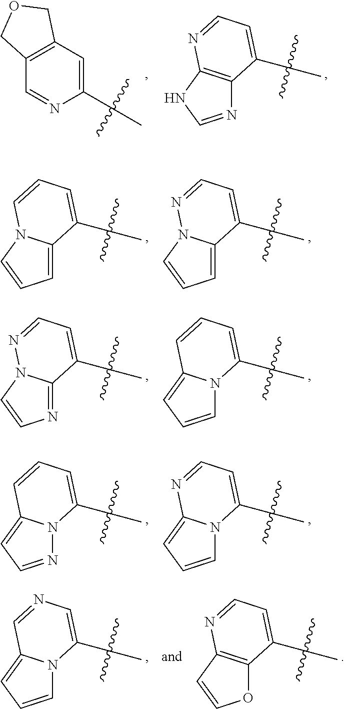

In some embodiments, the structural unit

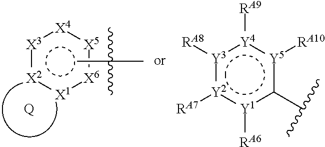

is selected from the following groups optionally substituted with one or more R^{4b}:

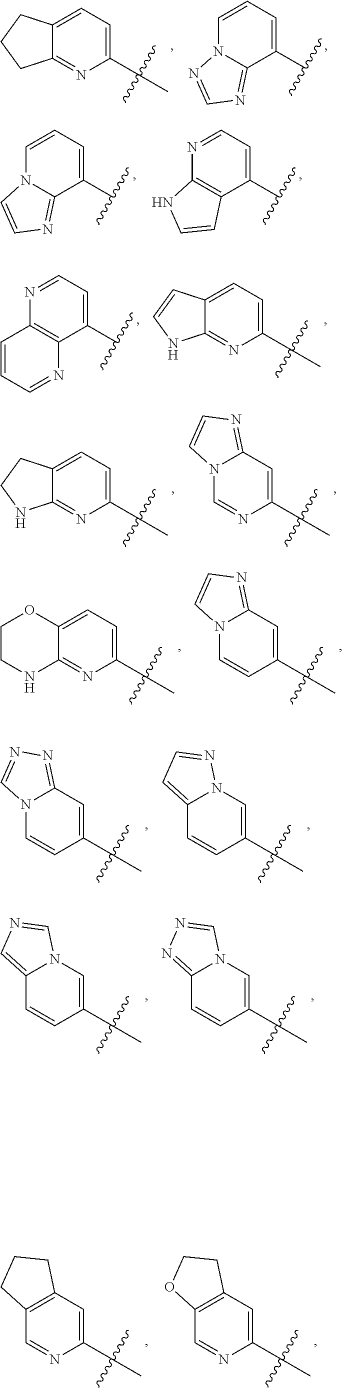

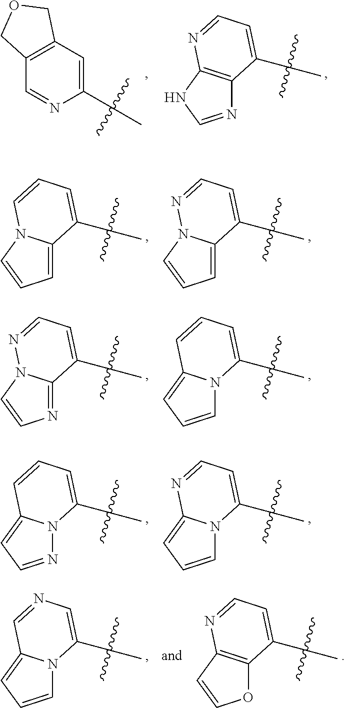

In some embodiments, the structural unit

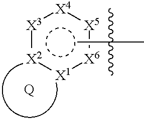

selected from

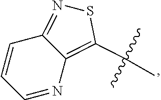 [SMILES: *c1snc2cccnc12]

wherein the

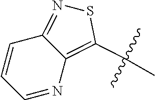 [SMILES: *c1snc2cccnc12]

is optionally substituted with one or more R^{4b}.

In some embodiments, the structural unit

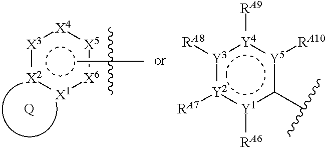

is selected from

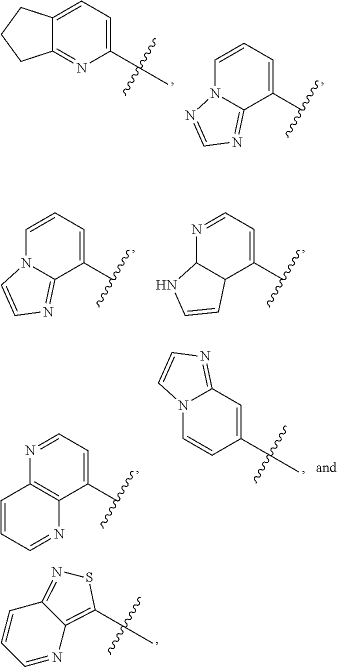

wherein the

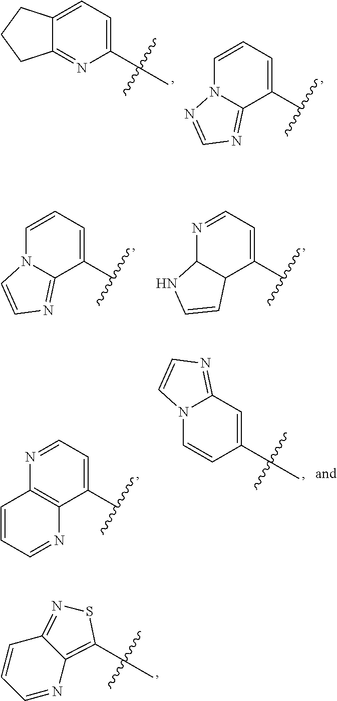

is optionally substituted with one or more R^{4b}.

In some embodiments, the structural unit

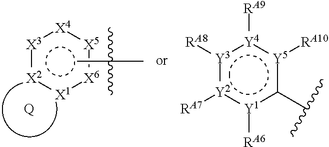

is selected from

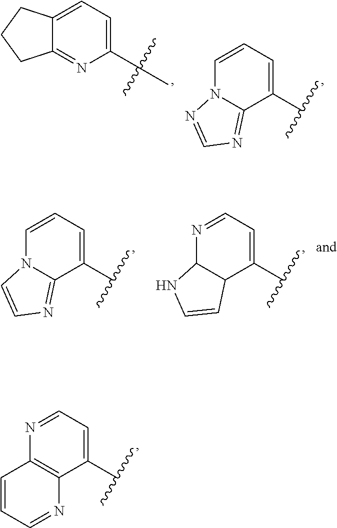 [SMILES: *c1ccc2c(n1)CCC2 ; *c1cccn2ncnc12 ; *c1cccn2ccnc12 ; *C1=CC=NC2NC=CC12 ; *c1ccnc2cccnc12]

wherein the

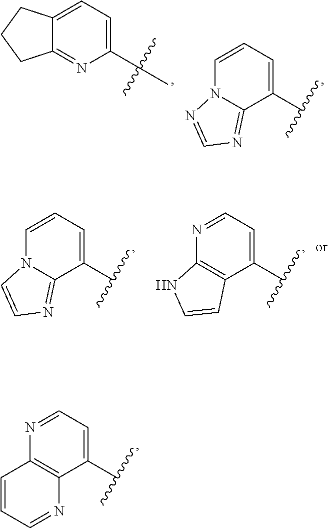 [SMILES: *c1ccc2c(n1)CCC2 ; *c1cccn2ncnc12 ; *c1cccn2ccnc12 ; *c1ccnc2[nH]ccc12 ; *c1ccnc2cccnc12]

is optionally substituted with one or more R^{4b}.

In some embodiments, ring A is selected from

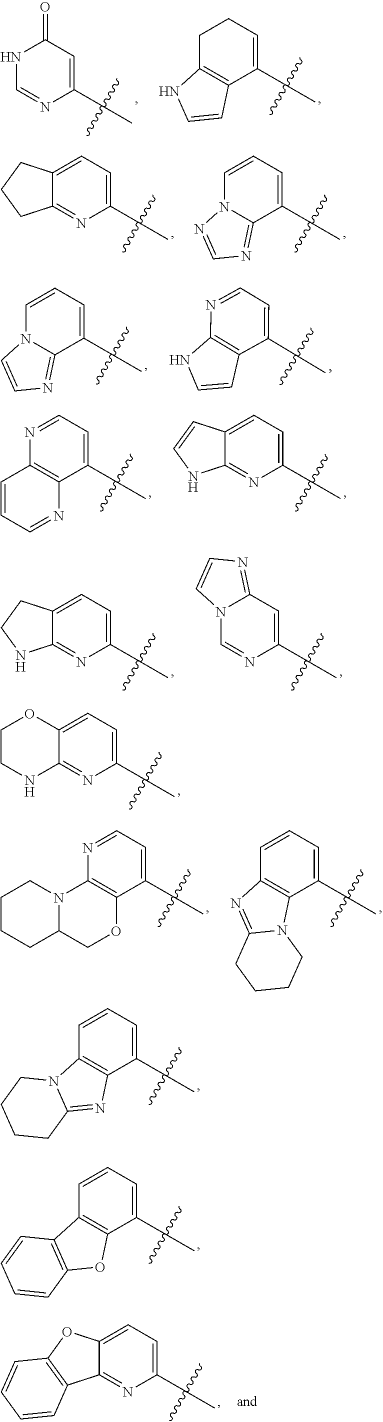

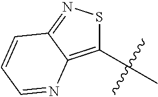 [SMILES: *c1snc2cccnc12]

wherein the

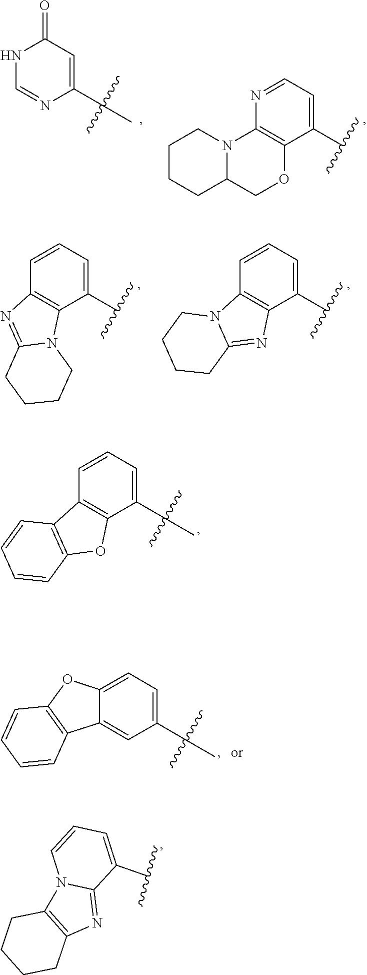

is optionally substituted with one or more R^{2b}, the

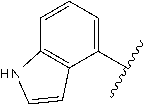 [SMILES: *c1cccc2[nH]ccc12]

is substituted with one or more R^{3a}, and the

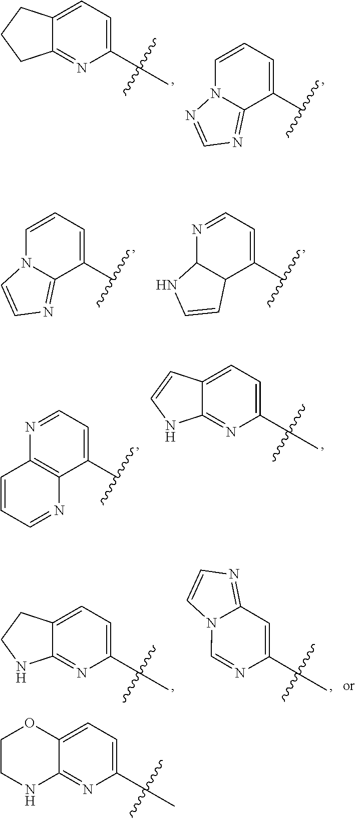

is optionally substituted with one or more R^{4b}.

In some embodiments, ring A is selected from

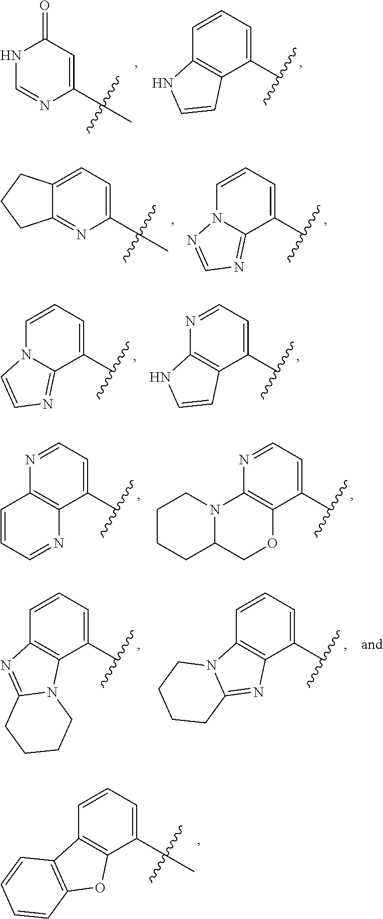

wherein the

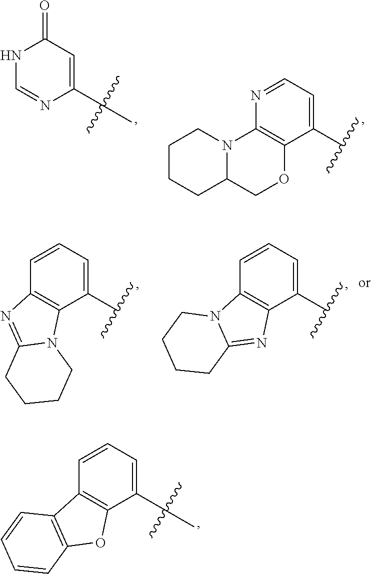 [SMILES: *c1cc(=O)[nH]cn1 ; *c1ccnc2c1OCC1CCCCN21 ; *c1cccc2nc3n(c12)CCCC3 ; *c1cccc2c1nc1n2CCCC1 ; *c1cccc2c1oc1ccccc12]

is optionally substituted with one or more R^{2b}, the

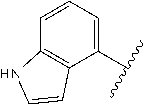 [SMILES: *c1cccc2[nH]ccc12]

is substituted with one or more R^{3a}, and the

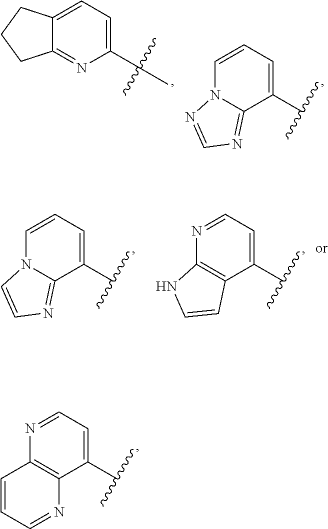 [SMILES: *c1ccc2c(n1)CCC2 ; *c1cccn2ncnc12 ; *c1cccn2ccnc12 ; *c1ccnc2[nH]ccc12 ; *c1ccnc2cccnc12]

is optionally substituted with one or more R^{4b}.

In some embodiments, R^{3a} is selected from C₃-C₆ cycloalkyl and 4-7 membered heterocyclyl.

In some embodiments, R^{3a} is selected from C₃-C₆ cycloalkyl, e.g., cyclobutyl.

In some embodiments, R^{3a} is selected from 4-7 membered heterocyclyl, e.g., piperidinyl, morpholinyl, or azabicyclo[3.1.0]hexyl. In some embodiments, R^{3a} is selected from cyclobutyl, piperidinyl, and morpholinyl.

In some embodiments, R^{3a} is selected from cyclobutyl and piperidinyl.

In some embodiments, R^{3a} is selected from piperidinyl.

In some embodiments, R^{3b} is selected from halogen and C₁-C₆ alkyl, e.g., F, C₁, and methyl.

In some embodiments, R^{3b} is selected from halogen, e.g., F.

In some embodiments, R^{2b} and/or R^{4b} are each independently selected from halogen, CN, C₁-C₆ alkyl, C₃-C₆ cycloalkyl, 4-10 membered heterocyclyl, C₂-C₄ alkenyl, C₅-C₁₀ cycloalkenyl, C(O)R^{d}, and O(C₁-C₄ alkyl), wherein the C₁-C₆ alkyl, C₃-C₆ cycloalkyl, 4-10 membered heterocyclyl, C₂-C₄ alkenyl, C₅-C₁₀ cycloalkenyl, or O(C₁-C₄ alkyl) is optionally substituted with one or more R^{c}.

In some embodiments, R^{2b} and/or R^{4b} are each independently selected from halogen, CN, C₁-C₆ alkyl, C₃-C₆ cycloalkyl, 4-10 membered heterocyclyl, C₂-C₄ alkenyl, and O(C₁-C₄ alkyl), wherein the C₁-C₆ alkyl, C₃-C₆ cycloalkyl, 4-10 membered heterocyclyl, C₂-C₄ alkenyl, or O(C₁-C₄ alkyl) is optionally substituted with one or more R^{c}.

In some embodiments, R^{2b} and/or R^{4b} are each independently selected from halogen, CN, C₁-C₆ alkyl, C₃-C₆ cycloalkyl, C₂-C₄ alkenyl, and 4-7 membered heterocyclyl, wherein the C₁-C₆ alkyl, C₃-C₆ cycloalkyl, C₂-C₄ alkenyl, or 4-7 membered heterocyclyl is optionally substituted with one or more R^{c}.

In some embodiments, R^{2b} and/or R^{4b} are each independently selected from halogen, CN, C₁-C₆ alkyl, C₃-C₆ cycloalkyl, 4-10 membered heterocyclyl, and O(C₁-C₄ alkyl), wherein the C₁-C₆ alkyl, C₃-C₆ cycloalkyl, 4-10 membered heterocyclyl, or O(C₁-C₄ alkyl) is optionally substituted with one or more R^{c}.

In some embodiments, R^{2b} and/or R^{4b} are each independently selected from halogen, CN, C₁-C₆ alkyl, C₃-C₆ cycloalkyl, and 4-7 membered heterocyclyl, wherein the C₁-C₆ alkyl, C₃-C₆ cycloalkyl, or 4-7 membered heterocyclyl is optionally substituted with one or more R^{c}.

In some embodiments, each R^{c} is independently selected from halogen, CN, OH, NH₂, C₁-C₄ alkyl, and O(C₁-C₄ alkyl).

In some embodiments, each R^{c} is independently selected from halogen, CN, OH, NH₂, and C₁-C₄ alkyl.

In some embodiments, each R^{c} is independently selected from halogen and O(C₁-C₄ alkyl).

In some embodiments, each R^{c} is independently selected from halogen.

In some embodiments, R^{d} is independently selected from O(C₁-C₄ alkyl), NH₂, NH(C₁-C₄ alkyl), and N(C₁-C₄ alkyl)₂.

In some embodiments, R^{d} is independently selected from O(C₁-C₄ alkyl) and NH(C₁-C₄ alkyl).

In some embodiments, R^{d} is independently selected from methoxy and NHCH₂CH₃.

In some embodiments, R^{2b} is independently selected from halogen, CN, C₁-C₆ alkyl, C₃-C₆ cycloalkyl, 4-10 membered heterocyclyl, C₅-C₁₀ cycloalkenyl, C(O)R^{d}, and O(C₁-C₄ alkyl), wherein the C₁-C₆ alkyl, C₃-C₆ cycloalkyl, 4-10 membered heterocyclyl, C₅-C₁₀ cycloalkenyl, or O(C₁-C₄ alkyl) is optionally substituted with one or more R^{c}, and R^{c} is selected from halogen and O(C₁-C₄ alkyl).

In some embodiments, R^{2b} is independently selected from halogen, CN, C₁-C₆ alkyl, C₃-C₆ cycloalkyl, 4-10 membered heterocyclyl, C₅-C₁₀ cycloalkenyl, C(O)R^{d}, and O(C₁-C₄ alkyl), wherein the C₁-C₆ alkyl, 4-10 membered heterocyclyl, and C₅-C₁₀ cycloalkenyl are optionally substituted with one or more R^{c}, and R^{c} is selected from halogen.

In some embodiments, R^{2b} is selected from cyano, halogen, C₁-C₄ alkyl, C₅-C₁₀ cycloalkenyl, C(O)R^{d}, O(C₁-C₄ alkyl), and piperidinyl, wherein the C₁-C₄ alkyl, O(C₁-C₄ alkyl), or piperidinyl is optionally substituted with one or more F.

In some embodiments, R^{2b} is selected from cyano, halogen, C₁-C₄ alkyl, O(C₁-C₄ alkyl), and piperidinyl, wherein the C₁-C₄ alkyl, O(C₁-C₄ alkyl), or piperidinyl is optionally substituted with one or more F.

In some embodiments, R^{2b} is selected from halogen, C₁-C₄ alkyl, and piperidinyl, wherein the C₁-C₄ alkyl or piperidinyl is optionally substituted with one or more F.

In some embodiments, R^{2b} is selected from C₁-C₄ alkyl and piperidinyl, wherein the C₁-C₄ alkyl or piperidinyl is optionally substituted with one or more F.

In some embodiments, R^{2b} is selected from CN, F, methyl, trifluoromethyl, methoxy, methoxymethyl, cyclopropyl,

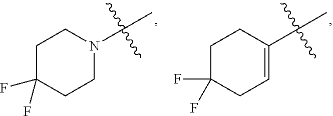 [SMILES: *N1CCC(F)(F)CC1 ; *C1=CCC(F)(F)CC1]

methoxycarbonyl, and ethylaminocarbonyl.

In some embodiments, R^{2b} is selected from CN, F, methyl, trifluoromethyl, methoxy, cyclopropyl,

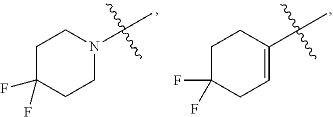 [SMILES: *N1CCC(F)(F)CC1 ; *C1=CCC(F)(F)CC1]

methoxycarbonyl, and ethylaminocarbonyl.

In some embodiments, R^{2b} is selected from CN, F, methyl, methoxy,

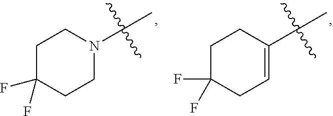 [SMILES: *N1CCC(F)(F)CC1 ; *C1=CCC(F)(F)CC1]

methoxycarbonyl, and ethylaminocarbonyl.

In some embodiments, R^{2b} is selected from CN, F, methyl, methoxy, and

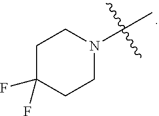 [SMILES: *N1CCC(F)(F)CC1]

In some embodiments, R^{2b} is selected from F, methyl, and

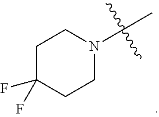 [SMILES: *N1CCC(F)(F)CC1]

In some embodiments, R^{2b} is selected from methyl and

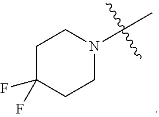 [SMILES: *N1CCC(F)(F)CC1]

In some embodiments, R^{4b} is selected from C₁-C₄ alkyl, O(C₁-C₄ alkyl), 4-10 membered heterocyclyl, and C₂-C₄ alkenyl, wherein the C₁-C₄ alkyl, O(C₁-C₄ alkyl), 4-10 membered heterocyclyl, or C₂-C₄ alkenyl is optionally substituted with one or more F.

In some embodiments, R^{4b} is selected from C₁-C₄ alkyl, O(C₁-C₄ alkyl), and 4-10 membered heterocyclyl, wherein the C₁-C₄ alkyl, O(C₁-C₄ alkyl), or 4-10 membered heterocyclyl is optionally substituted with one or more F.

In some embodiments, R^{4b} is selected from C₁-C₄ alkyl, wherein the C₁-C₄ alkyl is optionally substituted with one or more F.

In some embodiments, R^{4b} is selected from methyl, isopropyl, CF₃CH₂CH₂, CF₃CH₂CH₂O,

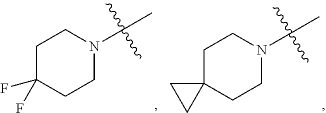 [SMILES: *N1CCC(F)(F)CC1 ; *N1CCC2(CC1)CC2]

and vinyl.

In some embodiments, R^{4b} is selected from methyl, isopropyl, CF₃CH₂CH₂, CF₃CH₂CH₂O,

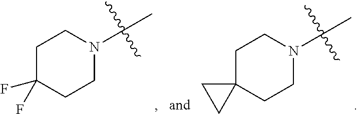 [SMILES: *N1CCC(F)(F)CC1 ; *N1CCC2(CC1)CC2]

In some embodiments, R^{4b} is selected from methyl, isopropyl, and CF₃CH₂CH₂.

In some embodiments, ring A is selected from

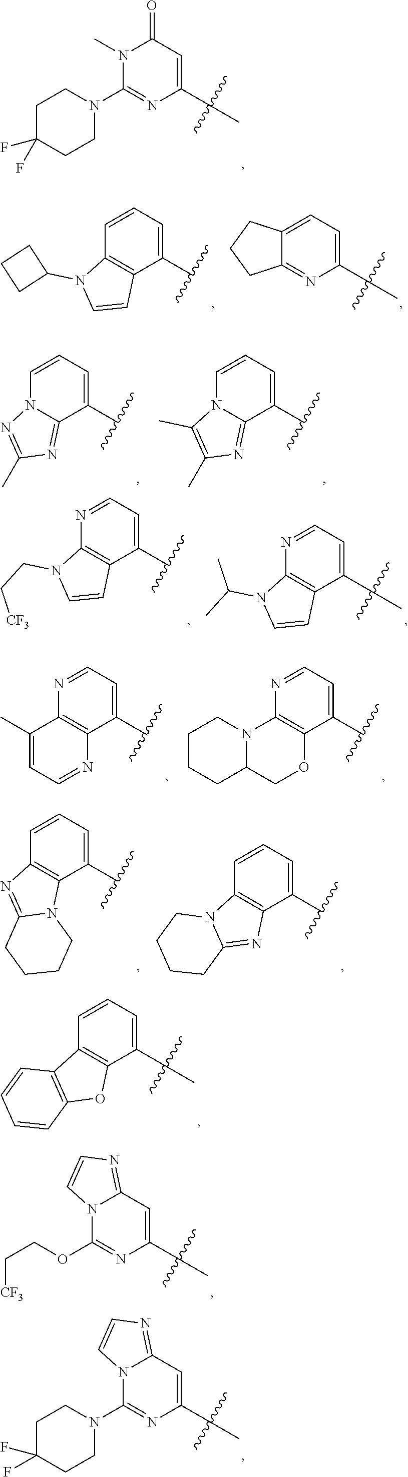

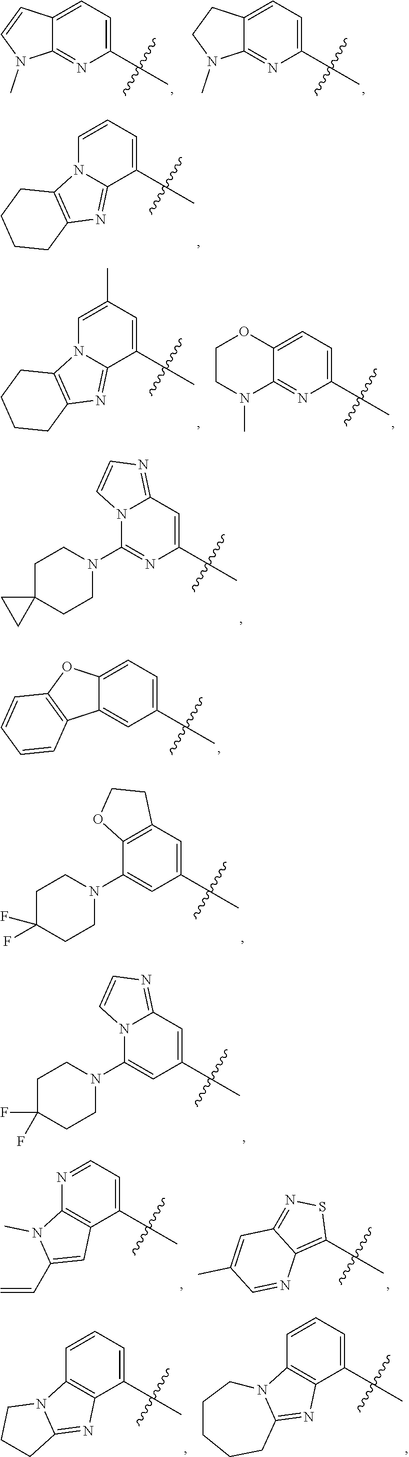

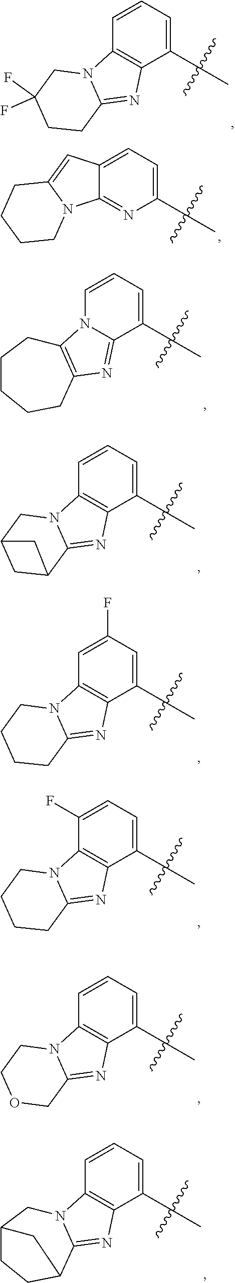

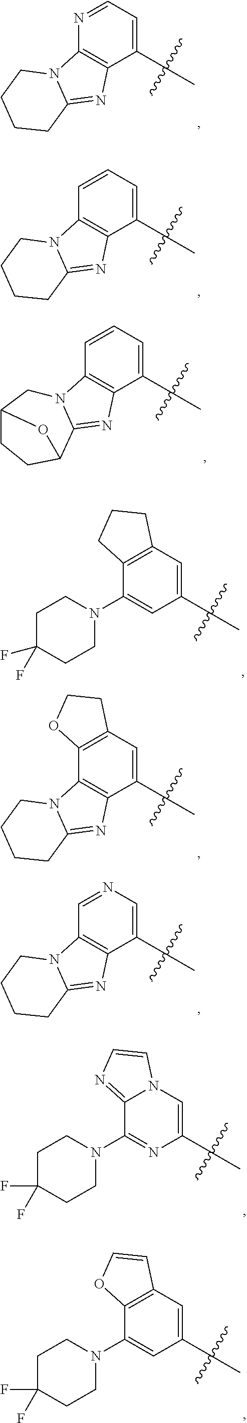

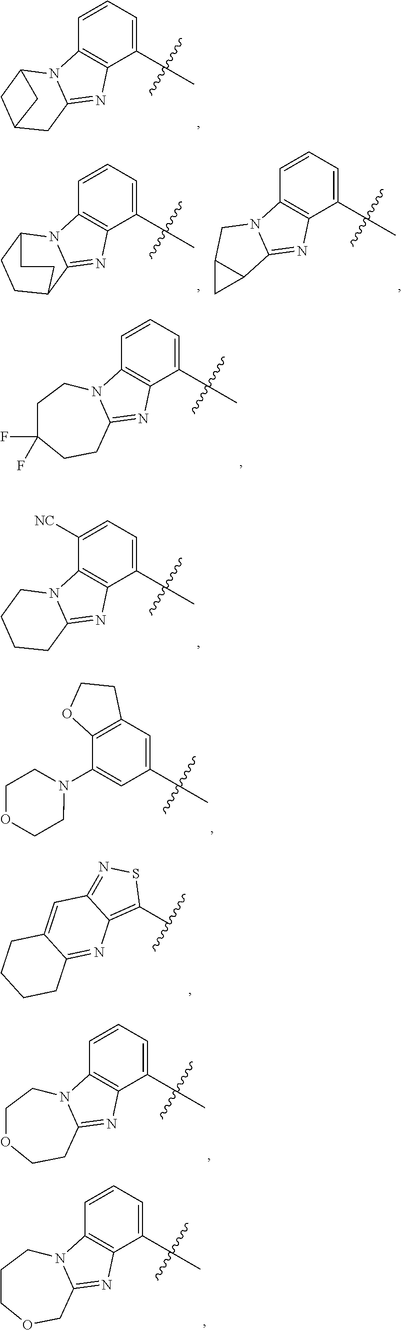

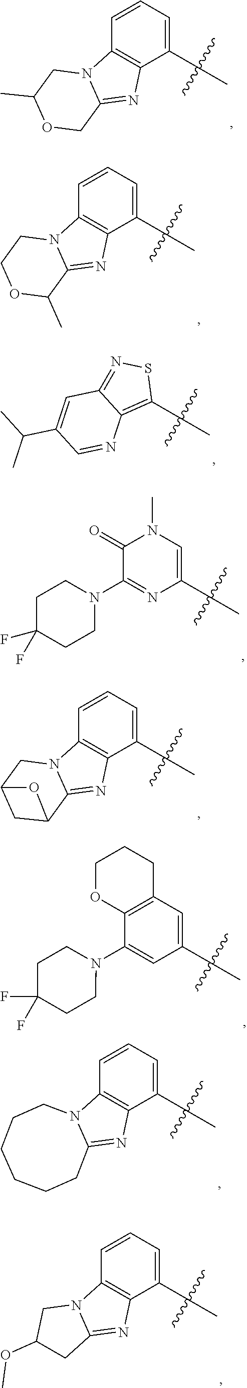

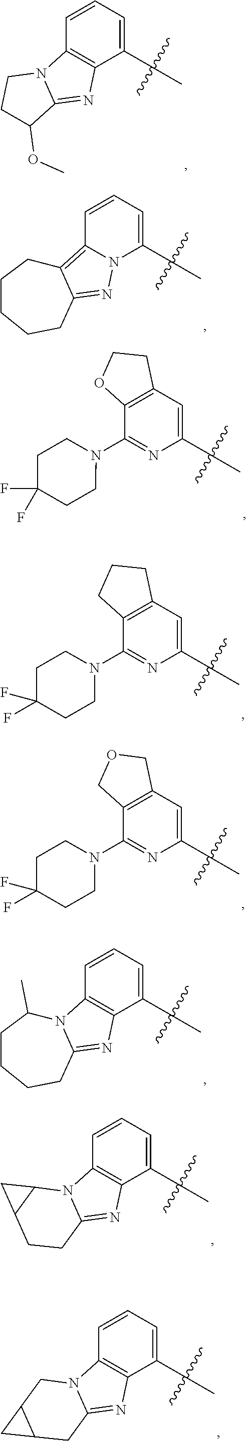

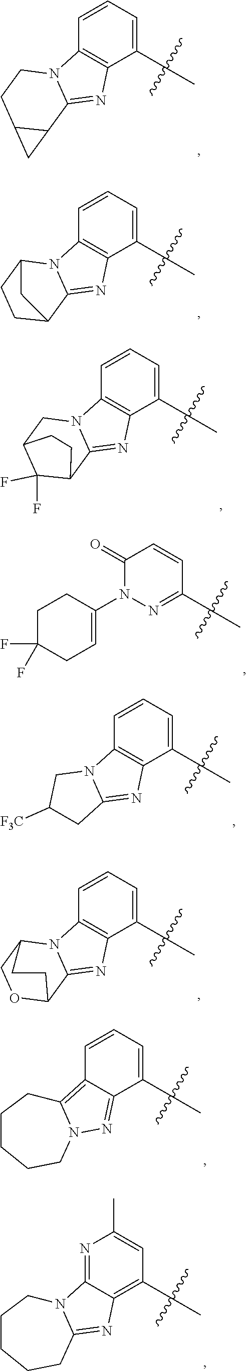

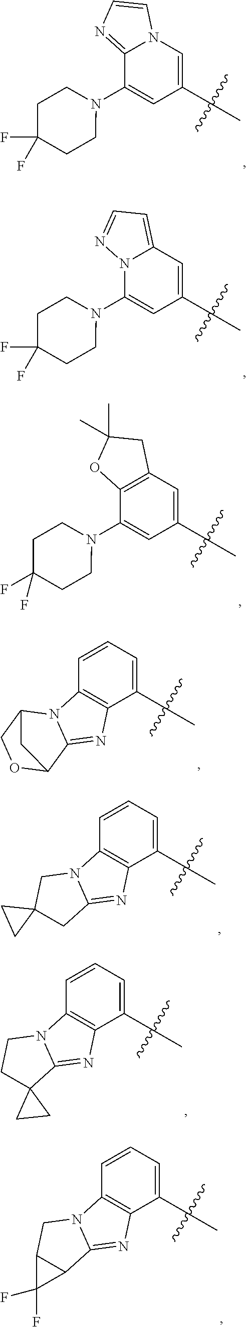

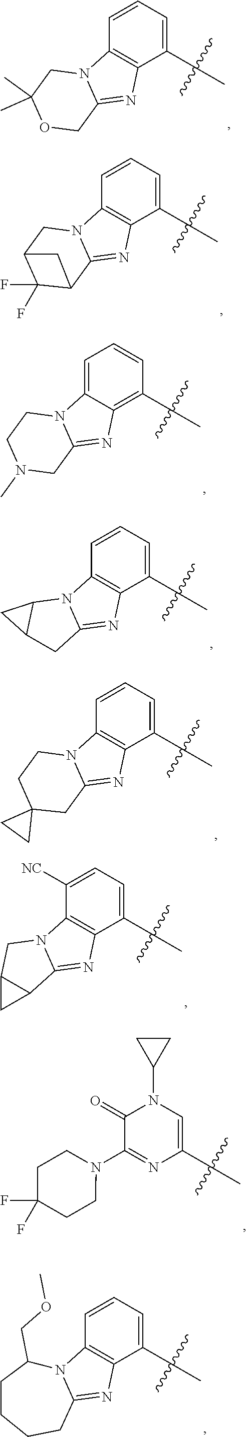

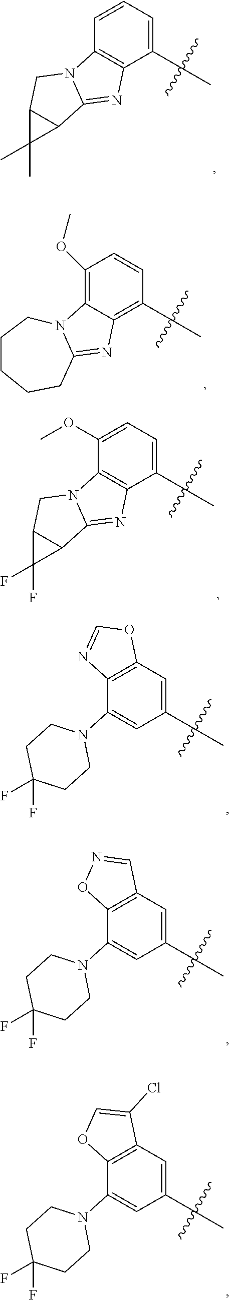

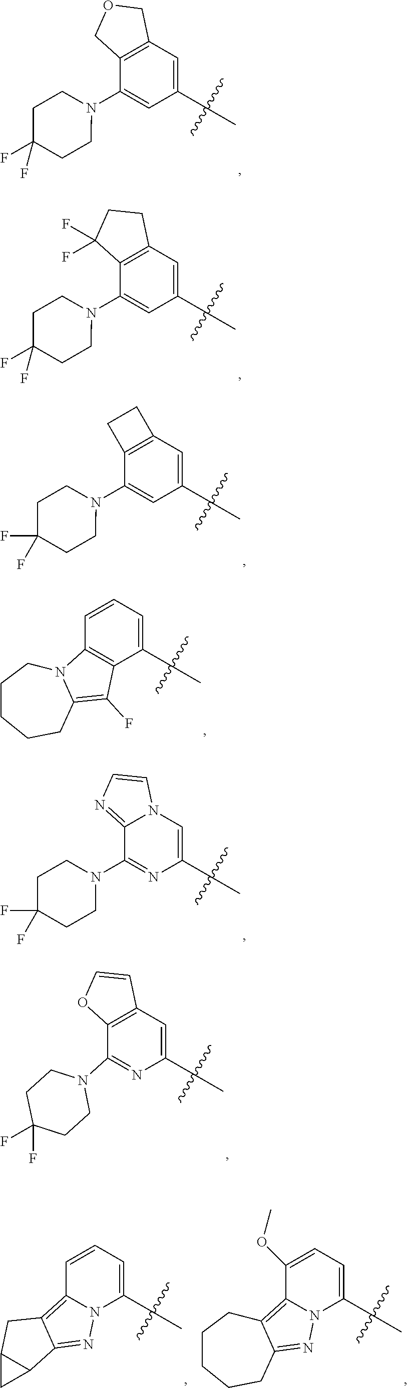

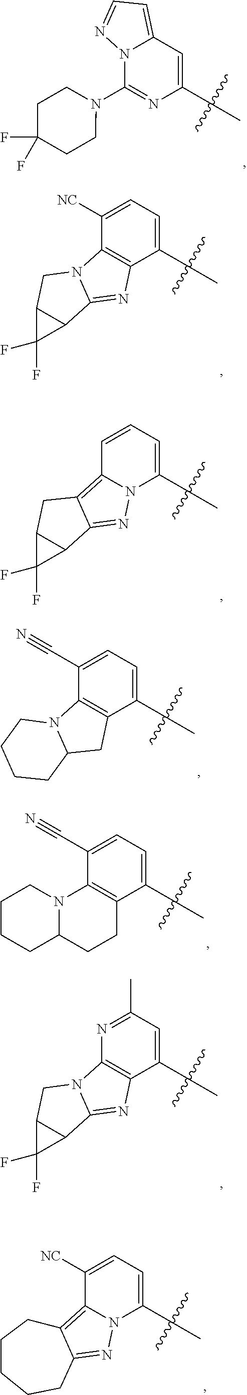

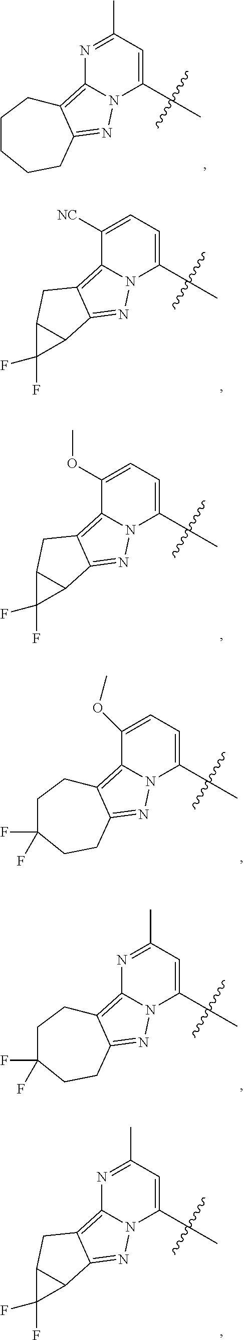 [SMILES: *c1cc(C)nc2c3c(nn12)CCCCC3 ; *c1ccc(C#N)c2c3c(nn12)C1C(C3)C1(F)F ; *c1ccc(OC)c2c3c(nn12)C1C(C3)C1(F)F ; *c1ccc(OC)c2c3c(nn12)CCC(F)(F)CC3 ; *c1cc(C)nc2c3c(nn12)CCC(F)(F)CC3 ; *c1cc(C)nc2c3c(nn12)C1C(C3)C1(F)F]

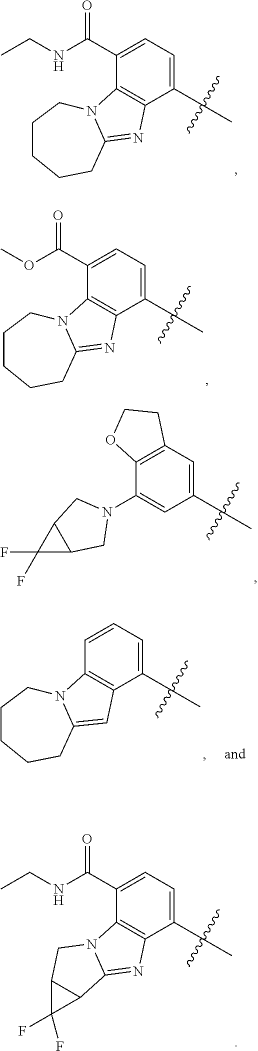

In some embodiments, ring A is selected from

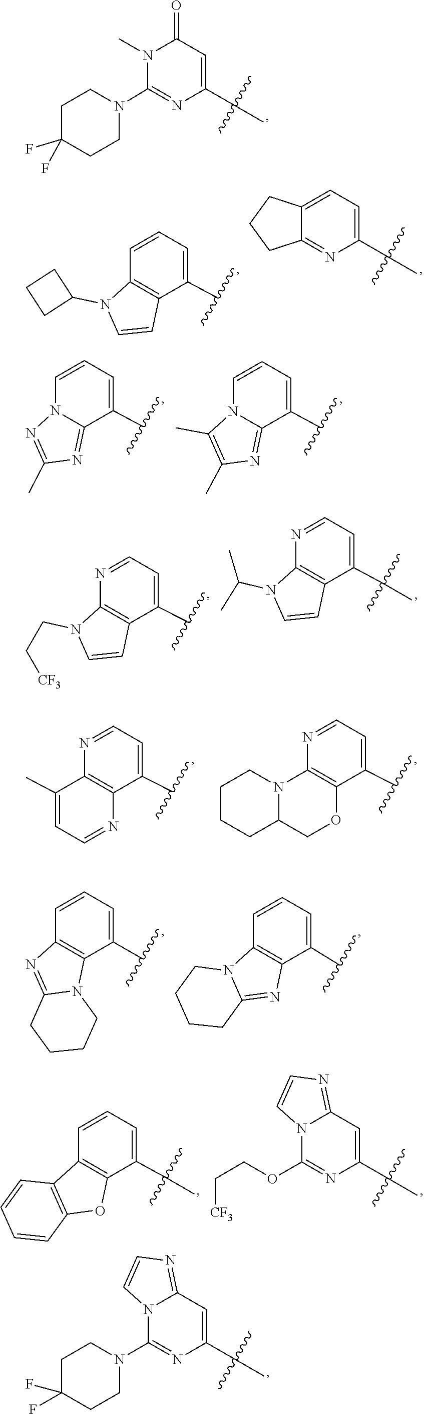

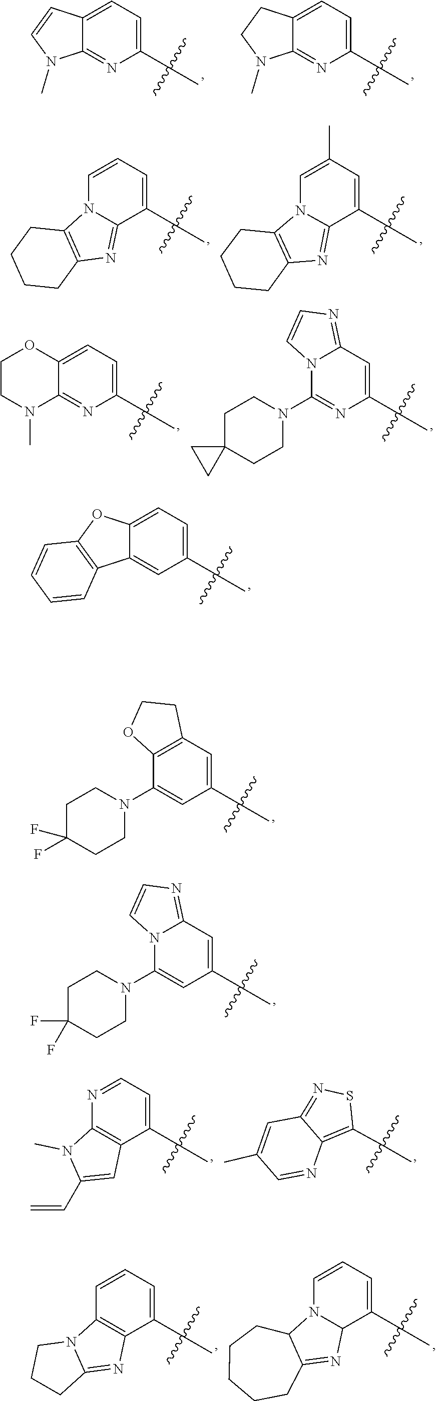

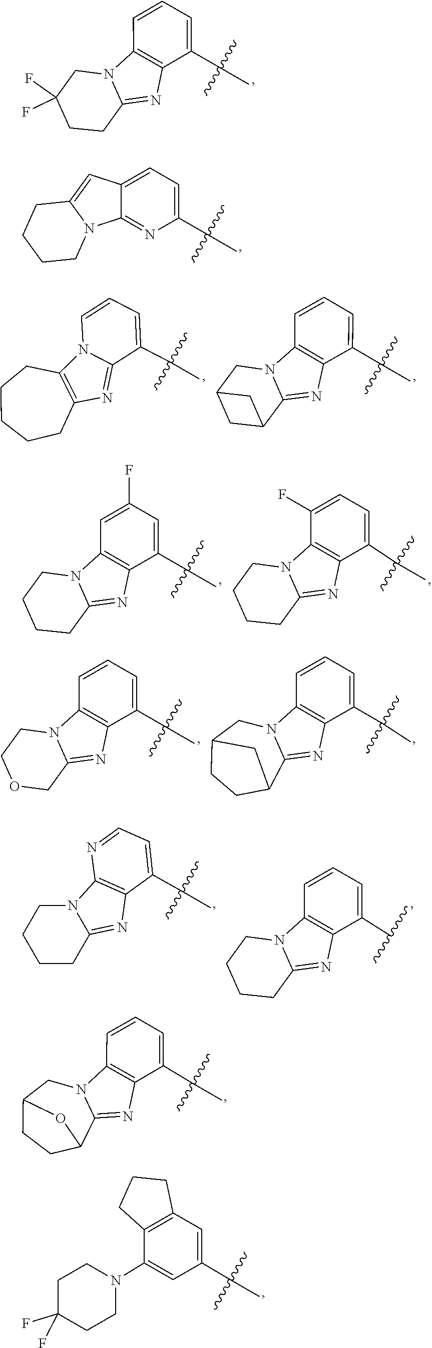

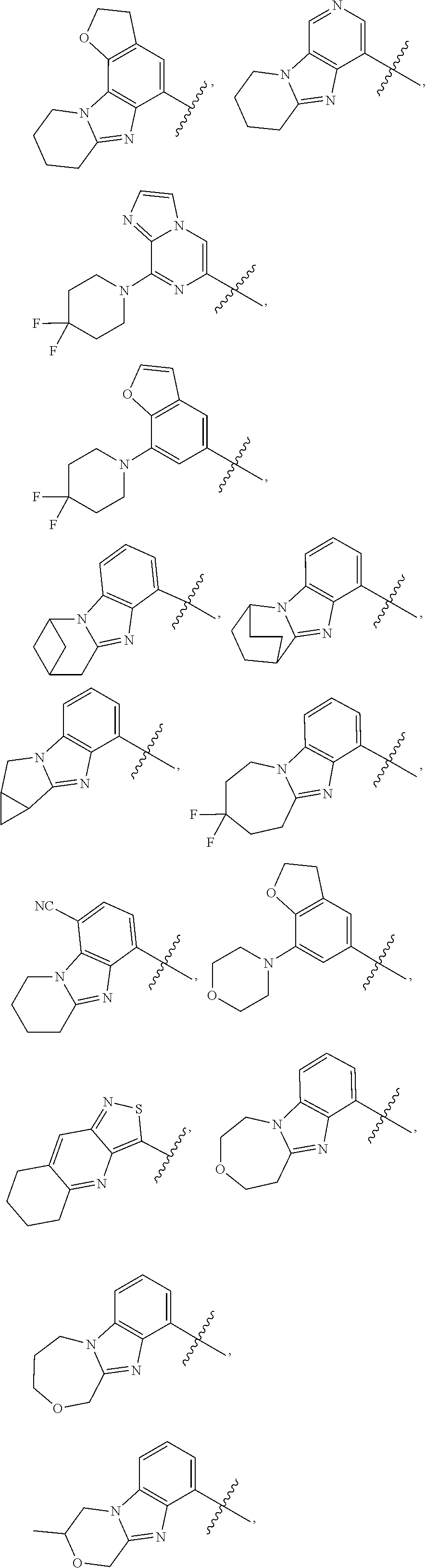

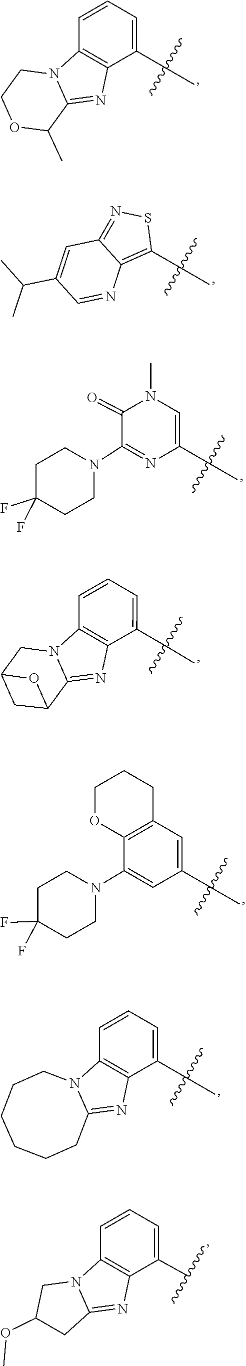

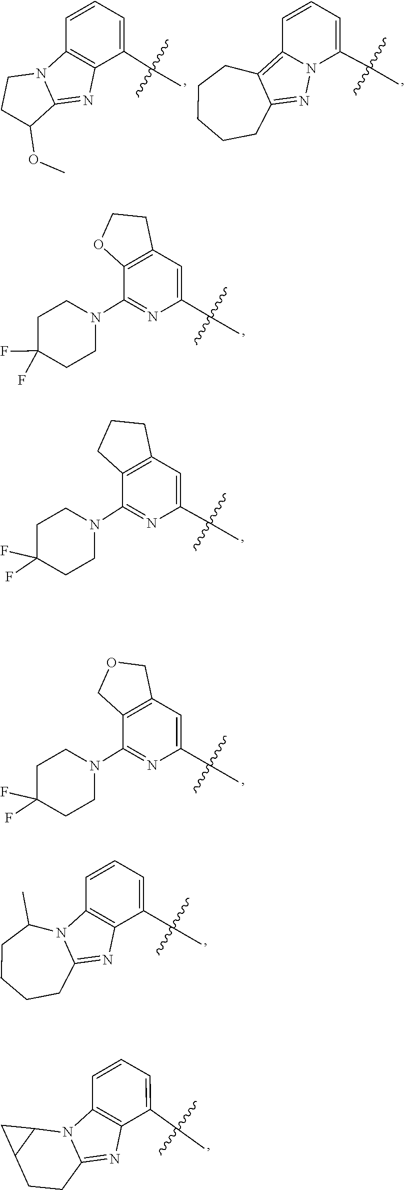

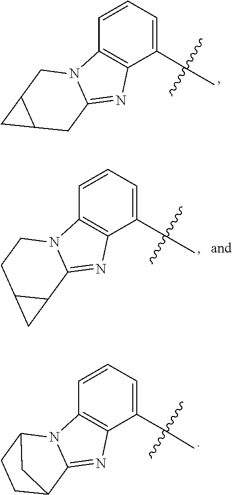 [SMILES: *c1cccc2c1nc1n2CC2CC2C1 ; *c1cccc2c1nc1n2CCC2CC12 ; *c1cccc2c1nc1n2C2CCC1C2]

In some embodiments, ring A is selected from

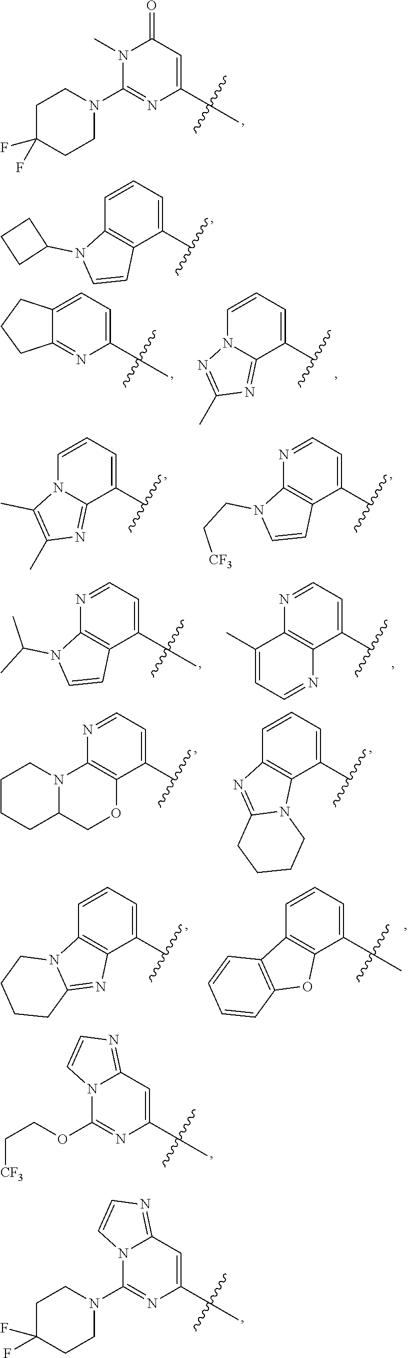

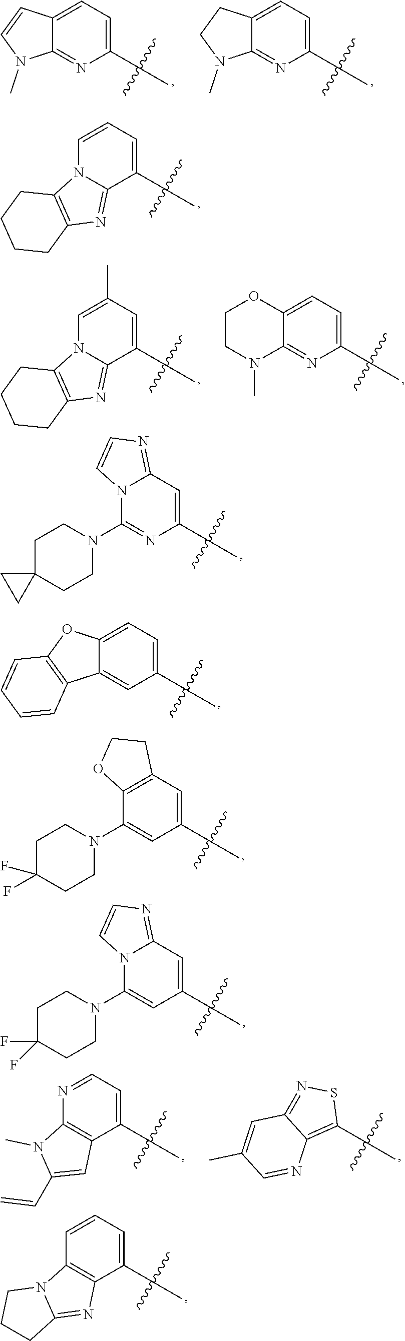

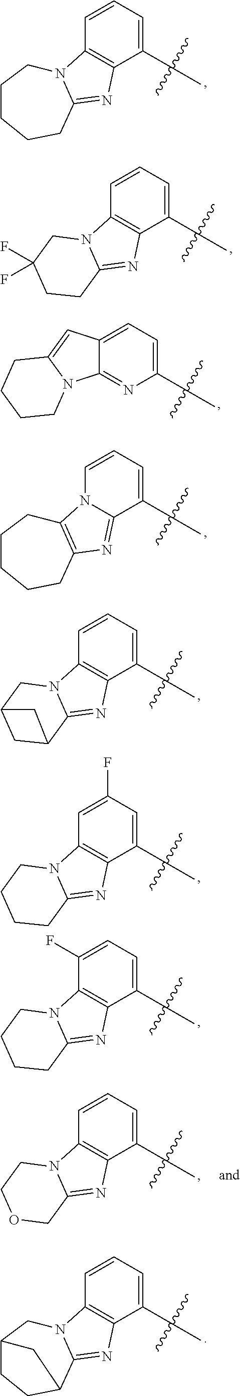

In some embodiments, ring A is selected from

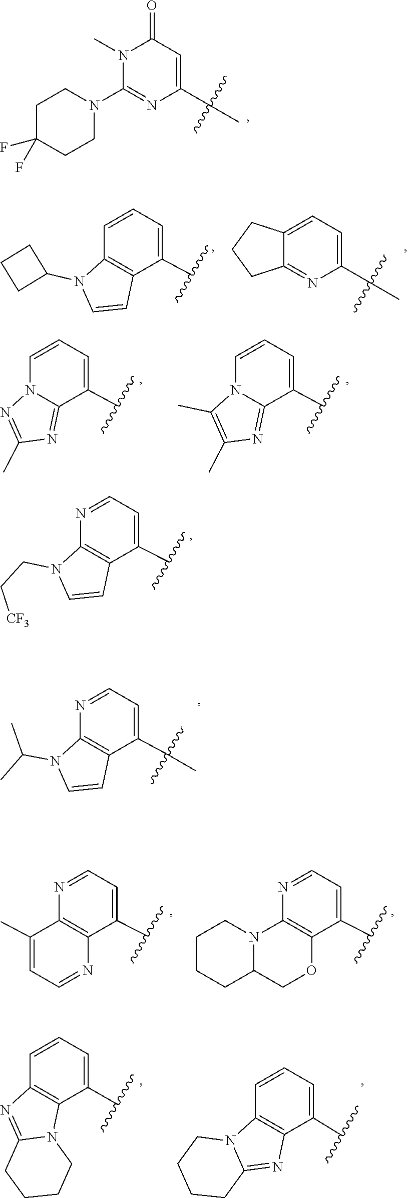

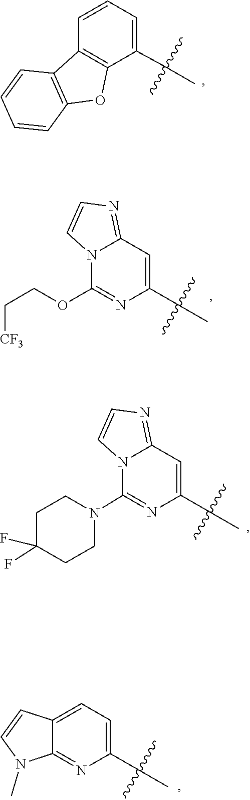 [SMILES: *c1cccc2c1oc1ccccc12 ; *c1cc2nccn2c(OCCC(F)(F)F)n1 ; *c1cc2nccn2c(N2CCC(F)(F)CC2)n1 ; *c1ccc2ccn(C)c2n1]

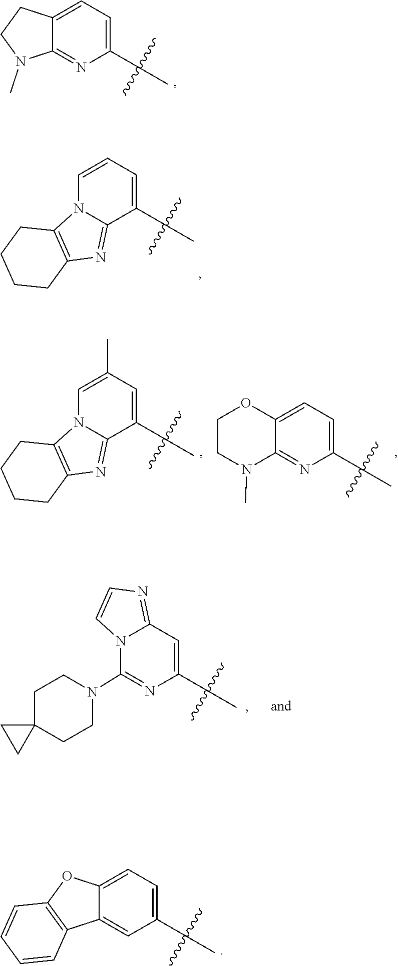 [SMILES: *c1ccc2c(n1)N(C)CC2 ; *c1cccn2c3c(nc12)CCCC3 ; *c1cc(C)cn2c3c(nc12)CCCC3 ; *c1ccc2c(n1)N(C)CCO2 ; *c1cc2nccn2c(N2CCC3(CC2)CC3)n1 ; *c1ccc2oc3ccccc3c2c1]

In some embodiments, ring A is selected from

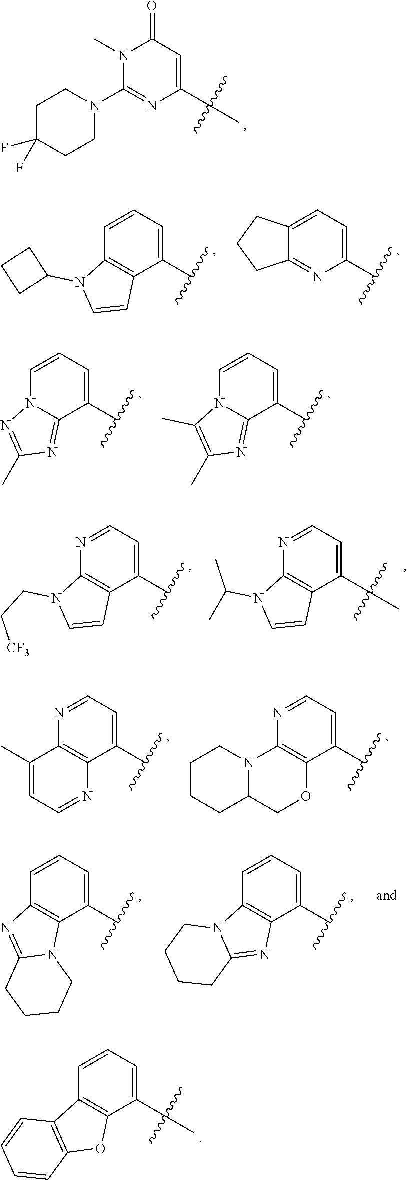

In some embodiments, R¹ is selected from H and C₁-C₄ alkyl.

In some embodiments, R¹ is selected from H.

In some embodiments, R² is selected from

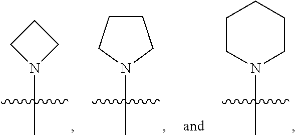

wherein the same carbon atom on the

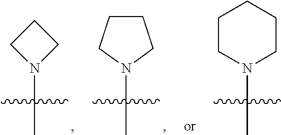

is simultaneously substituted with two R^{X}, and the two R^{x}, together with the carbon atom, form C₃-C₆ cycloalkyl, C₃-C₆ cycloalkenyl, or 4-7 membered heterocyclyl, wherein the C₃-C₆ cycloalkyl, C₃-C₆ cycloalkenyl, or 4-7 membered heterocyclyl is optionally substituted with one or more R^{xb}.

In some embodiments, R² is selected from

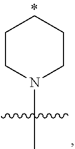 [SMILES: *N1CCCCC1]

the * carbon atom in the

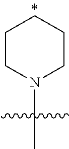 [SMILES: *N1CCCCC1]

is simultaneously substituted with two R^{x}, and the two R^{x}, together with the * carbon atom, form C₃-C₆ cycloalkyl, C₃-C₆ cycloalkenyl, or 4-7 membered heterocyclyl, wherein the C₃-C₆ cycloalkyl, C₃-C₆ cycloalkenyl, or 4-7 membered heterocyclyl is optionally substituted with one or more R^{xb}.

In some embodiments, R² is selected from

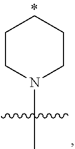 [SMILES: *N1CCCCC1]

the * carbon atom in the

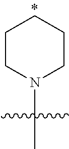 [SMILES: *N1CCCCC1]

is simultaneously substituted with two R^{x}, and the two R^{x}, together with the * carbon atom, form C₃-C₆ cycloalkyl, wherein the C₃-C₆ cycloalkyl is optionally substituted with one or more R^{xb}.

In some embodiments, R² is selected from

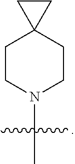 [SMILES: *N1CCC2(CC1)CC2]

In some embodiments, R³ is selected from L¹-L²-R^{y}, wherein L¹ is selected from —NH—, —O—, —S—, —S(═O)—, —SO₂—, —C(═O)—, —NHSO₂—, —NH(C═O)—, and

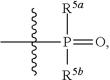

and R^{5a} and R^{5b} are each independently selected from C₁-C₄ alkyl, or R^{5a} and R^{5b}, together with the atom to which they are attached, form 4-7 membered heterocyclyl; L² is selected from a chemical bond and C₁-C₄ alkylene; R^{y} is selected from H, C₃-C₆ cycloalkyl, and 4-7 membered heterocyclyl; each C₁-C₄ alkyl, C₁-C₄ alkylene, C₃-C₆ cycloalkyl, or 4-7 membered heterocyclyl described above is optionally substituted with one or more halogen, CN, OH, or NH₂.

In some embodiments, R³ is selected from L¹-L²-R^{y}, wherein L¹ is selected from —NH—, —O—, —S—, —S(═O)—, —SO₂—, —C(═O)—, —NHSO₂—, and —NH(C═O)—, L² is selected from C₁-C₄ alkylene, and R^{y} is selected from H; the C₁-C₄ alkylene is optionally substituted with one or more halogen, CN, OH, or NH₂.

In some embodiments, R³ is selected from Li-L²-R^{y}, wherein Li is selected from —NHSO₂—, L² is selected from C₁-C₄ alkylene, and R^{y} is selected from H, wherein the C₁-C₄ alkylene is optionally substituted with one or more halogen or OH.

In some embodiments, R³ is selected from

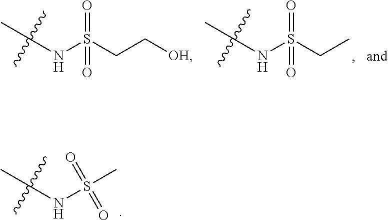

In some embodiments, R³ is selected from

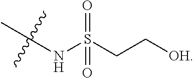 [SMILES: *NS(=O)(=O)CCO]

In some embodiments, X, Y, and Z are all selected from CH.

In another aspect, the present disclosure provides a compound of formula (II) or a pharmaceutically acceptable salt thereof:

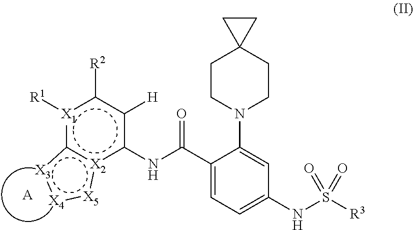

In formula (II):
A is a monocyclic or bicyclic ring selected from 4-10 membered carbon ring and 4-10 membered heterocycle, wherein the 4-10 membered carbon ring or 4-10 membered heterocycle is optionally substituted with one or more R^{b}, and each R^{b} is independently selected from halogen, OH, C₁-C₆ alkyl, O(C₁-C₄ alkyl), NH₂, NH(C₁-C₄ alkyl), and N(C₁-C₄ alkyl)₂, wherein the C₁-C₆ alkyl is optionally substituted with one or more groups independently selected from halogen, cyano, OH, NH₂, and O(C₁-C₄ alkyl);
X₁, X₂, X₃, X₄, and Xₛ are each independently CR^{a} or nitrogen, wherein R^{a} are each independently selected from being absent, H, and halogen;
R¹ is absent, H, C₁-C₆ alkyl, cyano, C₁-C₆ alkoxy, (CH₂)ᵣ—C(O)R^{d}, halogen, (CH₂)ᵣ—C₃-C₁₀ cycloalkyl, (CH₂)ᵣ—C₅-C₁₀ cycloalkenyl, (CH₂)ᵣ-4-10 membered heterocyclyl, (CH₂)ᵣ—C₆-C₁₀ aryl, or (CH₂)ᵣ-5-10 membered heteroaryl, wherein r is 0, 1, 2, or 3, and the C₁-C₆ alkyl, C₁-C₆ alkoxy, C₃-C₁₀ cycloalkyl, C₅-C₁₀ cycloalkenyl, 4-10 membered heterocyclyl, C₆-C₁₀ aryl, or 5-10 membered heteroaryl is optionally substituted with one or more groups independently selected from halogen, CN, OH, NH₂, C₁-C₆ alkyl, C₃-C₆ cycloalkyl, 4-7 membered heterocyclyl, O(C₁-C₄ alkyl), NH(C₁-C₄ alkyl), and N(C₁-C₄ alkyl)₂;
R² is H, C₁-C₆ alkyl, cyano, C₁-C₆ alkoxy, C(O)R^{d}, halogen, C₁-C₆ haloalkyl, or C₁-C₆ haloalkoxy; R^{d} is selected from OH, C₁-C₆ alkyl, O(C₁-C₄ alkyl), NH₂, NH(C₁-C₄ alkyl), and N(C₁-C₄ alkyl)₂; and
R³ is selected from C₁-C₆ alkyl optionally substituted with one or more halogen or OH.

In some embodiments, the compounds of formula (II) and its subformula (III) below do not include the following compounds:

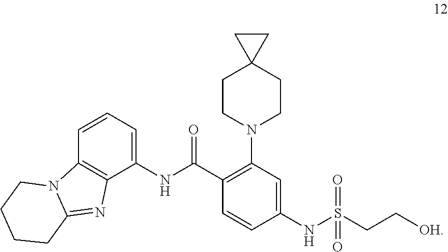 [SMILES: O=C(Nc1cccc2c1nc1n2CCCC1)c1ccc(NS(=O)(=O)CCO)cc1N1CCC2(CC1)CC2]

In some embodiments, the compounds of formula (II) and its subformula (III) below do not include the following compounds:

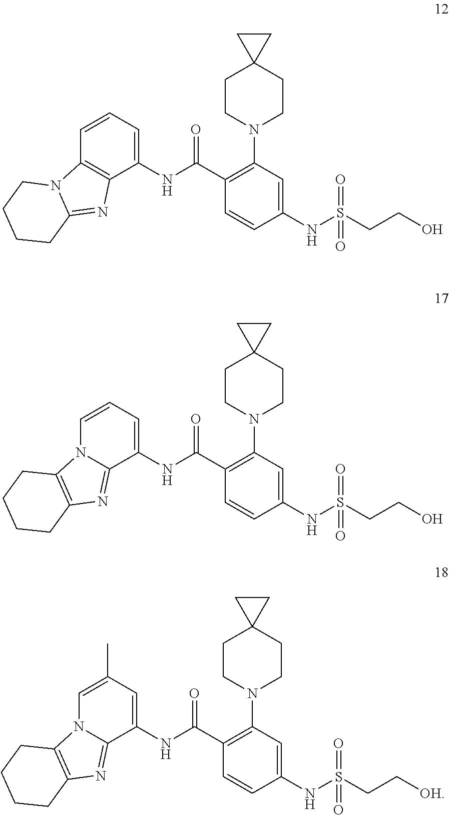 [SMILES: O=C(Nc1cccc2c1nc1n2CCCC1)c1ccc(NS(=O)(=O)CCO)cc1N1CCC2(CC1)CC2 ; O=C(Nc1cccn2c3c(nc12)CCCC3)c1ccc(NS(=O)(=O)CCO)cc1N1CCC2(CC1)CC2 ; Cc1cc(NC(=O)c2ccc(NS(=O)(=O)CCO)cc2N2CCC3(CC2)CC3)c2nc3c(n2c1)CCCC3]

In some embodiments, the compounds of formula (II) and its subformula (III) below do not include the following compounds:

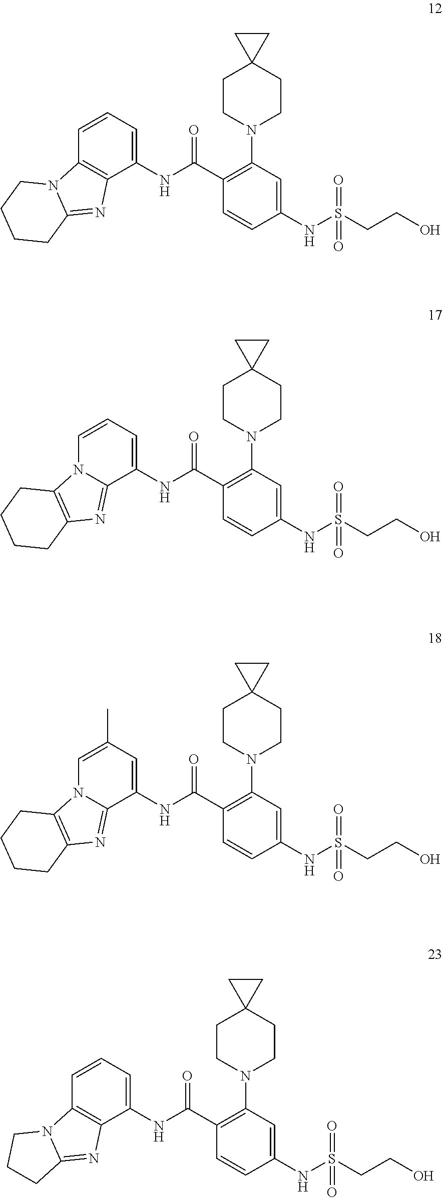

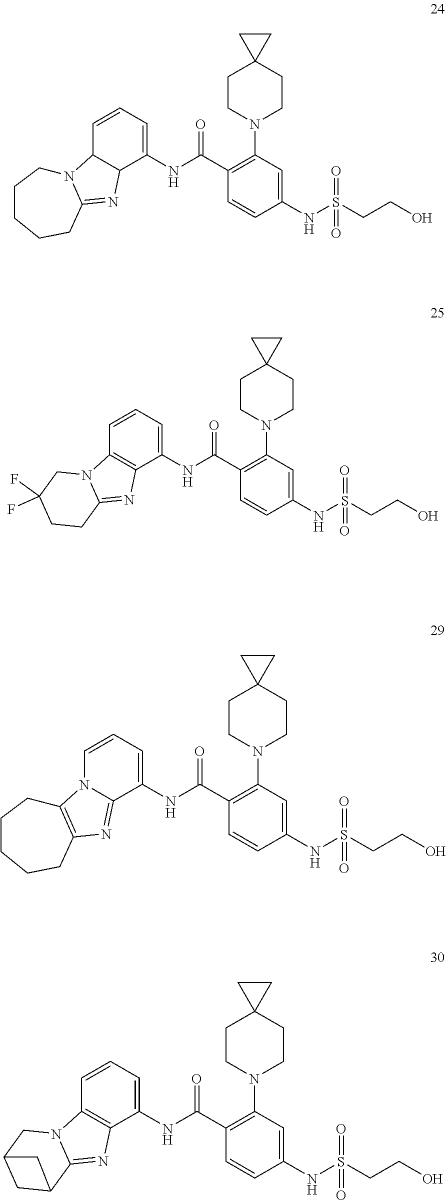

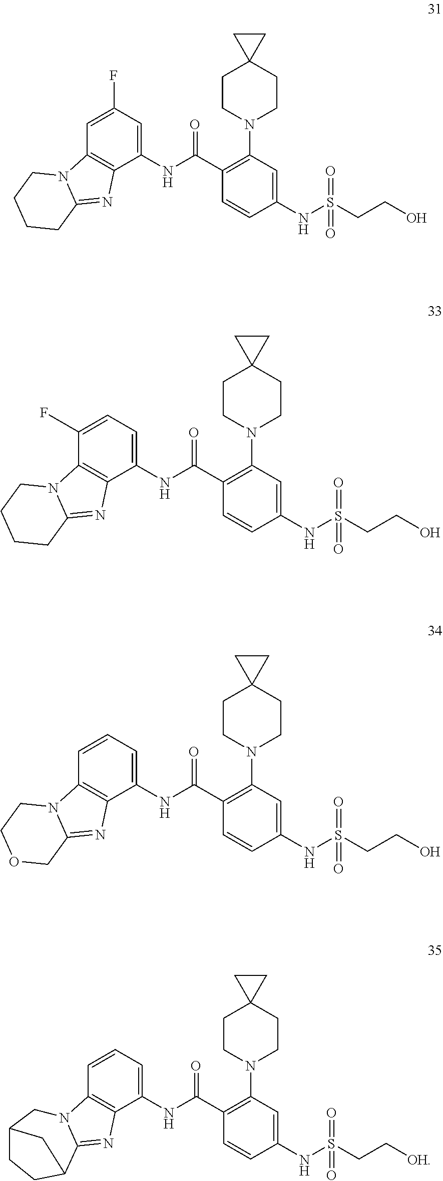

In some embodiments, the compounds of formula (II) and its subformula (III) below do not include the following compounds:

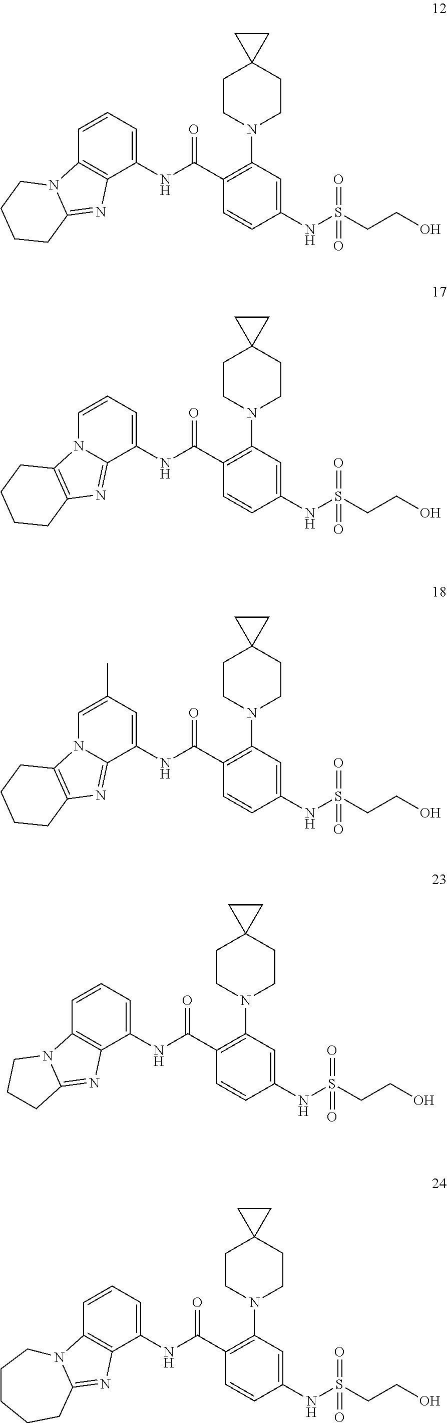 [SMILES: O=C(Nc1cccc2c1nc1n2CCCC1)c1ccc(NS(=O)(=O)CCO)cc1N1CCC2(CC1)CC2 ; O=C(Nc1cccn2c3c(nc12)CCCC3)c1ccc(NS(=O)(=O)CCO)cc1N1CCC2(CC1)CC2 ; Cc1cc(NC(=O)c2ccc(NS(=O)(=O)CCO)cc2N2CCC3(CC2)CC3)c2nc3c(n2c1)CCCC3 ; O=C(Nc1cccc2c1nc1n2CCC1)c1ccc(NS(=O)(=O)CCO)cc1N1CCC2(CC1)CC2 ; O=C(Nc1cccc2c1nc1n2CCCCC1)c1ccc(NS(=O)(=O)CCO)cc1N1CCC2(CC1)CC2]

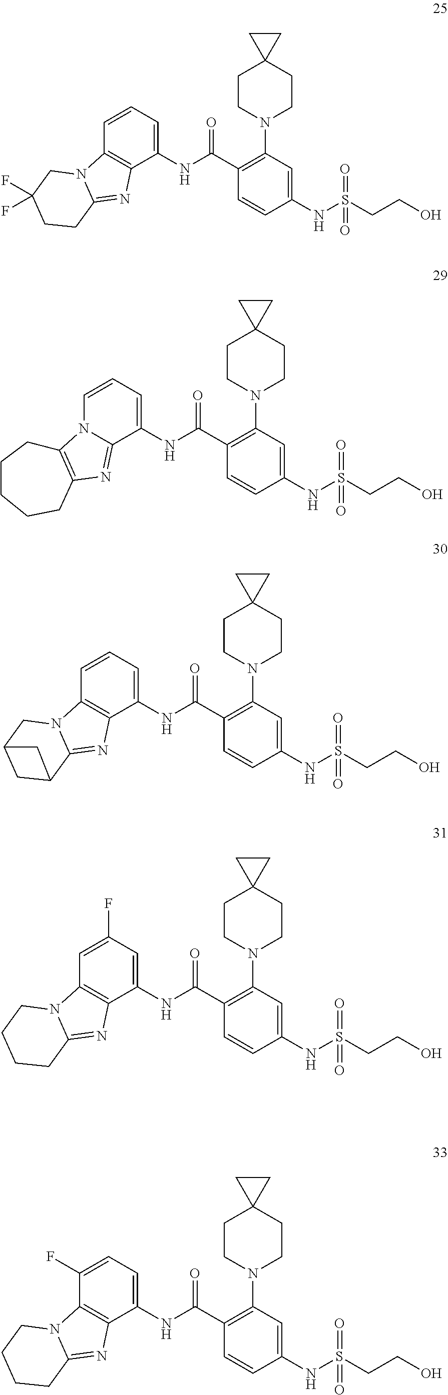 [SMILES: O=C(Nc1cccc2c1nc1n2CC(F)(F)CC1)c1ccc(NS(=O)(=O)CCO)cc1N1CCC2(CC1)CC2 ; O=C(Nc1cccn2c3c(nc12)CCCCC3)c1ccc(NS(=O)(=O)CCO)cc1N1CCC2(CC1)CC2 ; O=C(Nc1cccc2c1nc1n2CC2CC1C2)c1ccc(NS(=O)(=O)CCO)cc1N1CCC2(CC1)CC2 ; O=C(Nc1cc(F)cc2c1nc1n2CCCC1)c1ccc(NS(=O)(=O)CCO)cc1N1CCC2(CC1)CC2 ; O=C(Nc1ccc(F)c2c1nc1n2CCCC1)c1ccc(NS(=O)(=O)CCO)cc1N1CCC2(CC1)CC2]

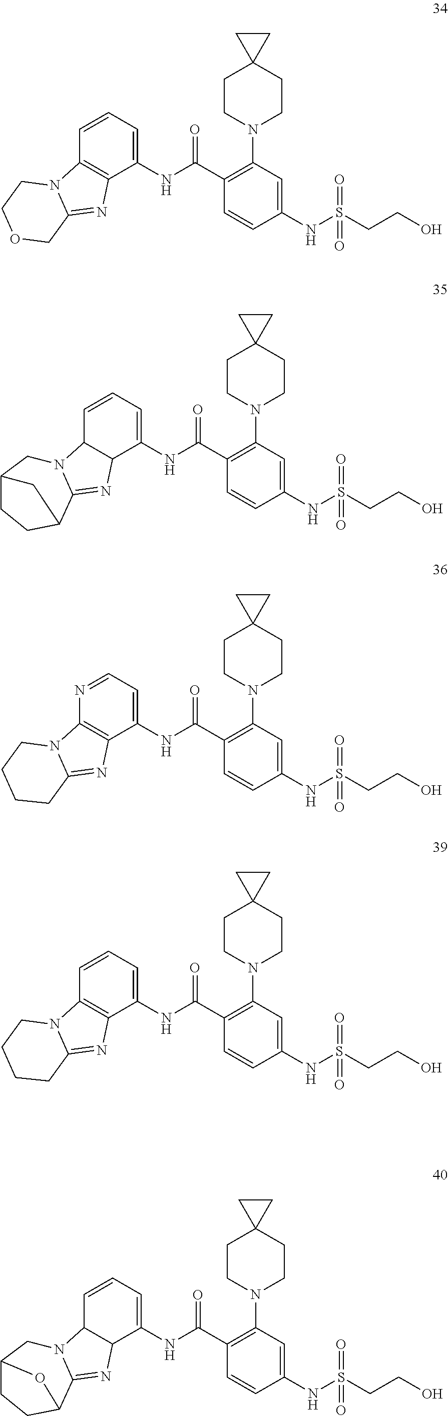 [SMILES: O=C(Nc1cccc2c1nc1n2CCOC1)c1ccc(NS(=O)(=O)CCO)cc1N1CCC2(CC1)CC2 ; O=C(NC1=CC=CC2C1N=C1C3CCC(C3)CN12)c1ccc(NS(=O)(=O)CCO)cc1N1CCC2(CC1)CC2 ; O=C(Nc1ccnc2c1nc1n2CCCC1)c1ccc(NS(=O)(=O)CCO)cc1N1CCC2(CC1)CC2 ; O=C(Nc1cccc2c1nc1n2CCCC1)c1ccc(NS(=O)(=O)CCO)cc1N1CCC2(CC1)CC2 ; O=C(NC1=CC=CC2C1N=C1C3CCC(CN12)O3)c1ccc(NS(=O)(=O)CCO)cc1N1CCC2(CC1)CC2]

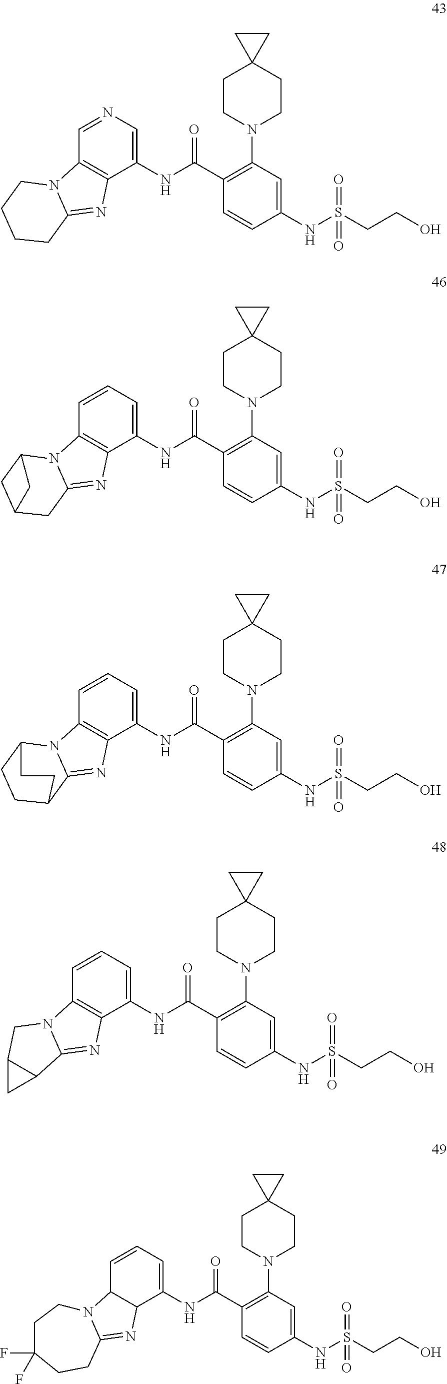 [SMILES: O=C(Nc1cncc2c1nc1n2CCCC1)c1ccc(NS(=O)(=O)CCO)cc1N1CCC2(CC1)CC2 ; O=C(Nc1cccc2c1nc1n2C2CC(C1)C2)c1ccc(NS(=O)(=O)CCO)cc1N1CCC2(CC1)CC2 ; O=C(Nc1cccc2c1nc1n2C2CCC1CC2)c1ccc(NS(=O)(=O)CCO)cc1N1CCC2(CC1)CC2 ; O=C(Nc1cccc2c1nc1n2CC2CC12)c1ccc(NS(=O)(=O)CCO)cc1N1CCC2(CC1)CC2 ; O=C(NC1=CC=CC2C1N=C1CCC(F)(F)CCN12)c1ccc(NS(=O)(=O)CCO)cc1N1CCC2(CC1)CC2]

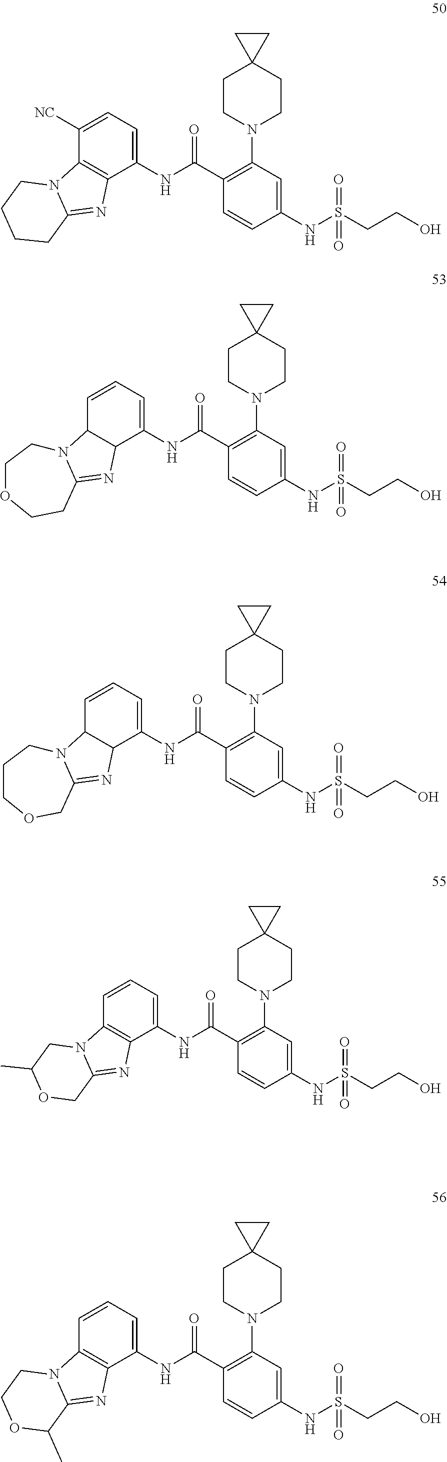 [SMILES: N#Cc1ccc(NC(=O)c2ccc(NS(=O)(=O)CCO)cc2N2CCC3(CC2)CC3)c2nc3n(c12)CCCC3 ; O=C(NC1=CC=CC2C1N=C1CCOCCN12)c1ccc(NS(=O)(=O)CCO)cc1N1CCC2(CC1)CC2 ; O=C(NC1=CC=CC2C1N=C1COCCCN12)c1ccc(NS(=O)(=O)CCO)cc1N1CCC2(CC1)CC2 ; CC1Cn2c(nc3c(NC(=O)c4ccc(NS(=O)(=O)CCO)cc4N4CCC5(CC4)CC5)cccc32)CO1 ; CC1OCCn2c1nc1c(NC(=O)c3ccc(NS(=O)(=O)CCO)cc3N3CCC4(CC3)CC4)cccc12]

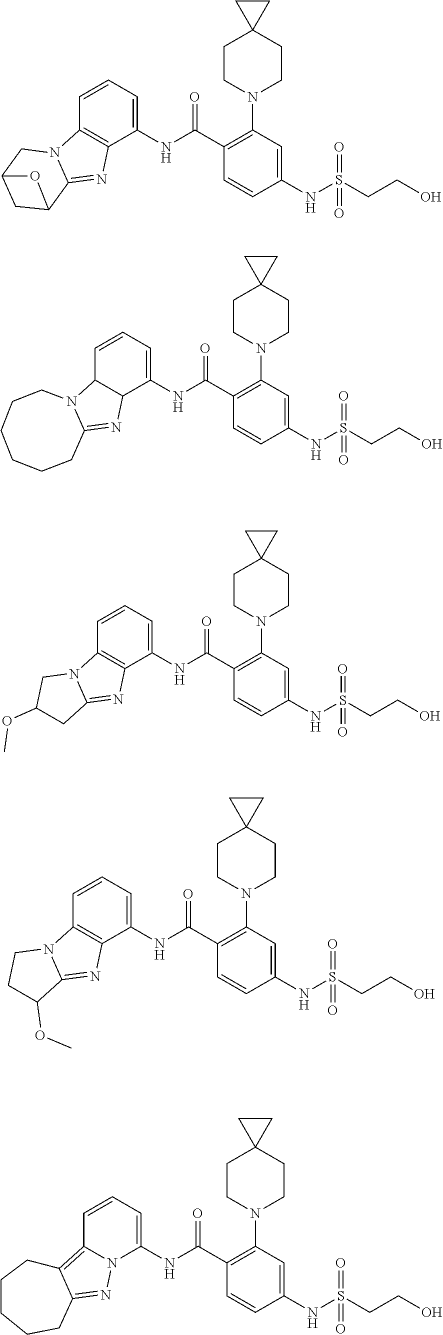 [SMILES: O=C(Nc1cccc2c1nc1n2CC2CC1O2)c1ccc(NS(=O)(=O)CCO)cc1N1CCC2(CC1)CC2 ; O=C(NC1=CC=CC2C1N=C1CCCCCCN12)c1ccc(NS(=O)(=O)CCO)cc1N1CCC2(CC1)CC2 ; COC1Cc2nc3c(NC(=O)c4ccc(NS(=O)(=O)CCO)cc4N4CCC5(CC4)CC5)cccc3n2C1 ; COC1CCn2c1nc1c(NC(=O)c3ccc(NS(=O)(=O)CCO)cc3N3CCC4(CC3)CC4)cccc12 ; O=C(Nc1cccc2c3c(nn12)CCCCC3)c1ccc(NS(=O)(=O)CCO)cc1N1CCC2(CC1)CC2]

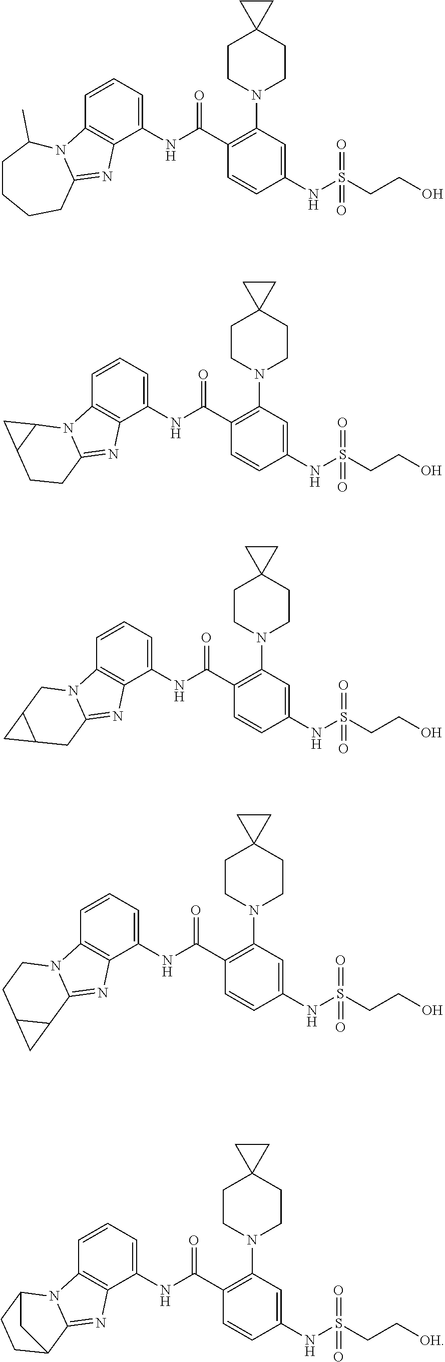 [SMILES: CC1CCCCc2nc3c(NC(=O)c4ccc(NS(=O)(=O)CCO)cc4N4CCC5(CC4)CC5)cccc3n21 ; O=C(Nc1cccc2c1nc1n2C2CC2CC1)c1ccc(NS(=O)(=O)CCO)cc1N1CCC2(CC1)CC2 ; O=C(Nc1cccc2c1nc1n2CC2CC2C1)c1ccc(NS(=O)(=O)CCO)cc1N1CCC2(CC1)CC2 ; O=C(Nc1cccc2c1nc1n2CCC2CC12)c1ccc(NS(=O)(=O)CCO)cc1N1CCC2(CC1)CC2 ; O=C(Nc1cccc2c1nc1n2C2CCC1C2)c1ccc(NS(=O)(=O)CCO)cc1N1CCC2(CC1)CC2]

In some embodiments, when one of R¹ and R² is H, the other is not H, methyl, CN, or halogen.

In some embodiments, A is a monocyclic or bicyclic ring selected from 4-10 membered carbon ring and 4-10 membered heterocycle, wherein the 4-10 membered carbon ring or 4-10 membered heterocycle is optionally substituted with one or more R^{b}, and each R^{b} is independently selected from F and C₁-C₄ alkyl, wherein the C₁-C₄ alkyl is optionally substituted with one or more groups independently selected from F and O(C₁-C₄ alkyl).

In some embodiments, the structural unit

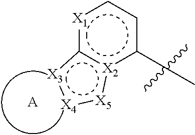

is selected from the following groups optionally substituted with one or more R^{b}:

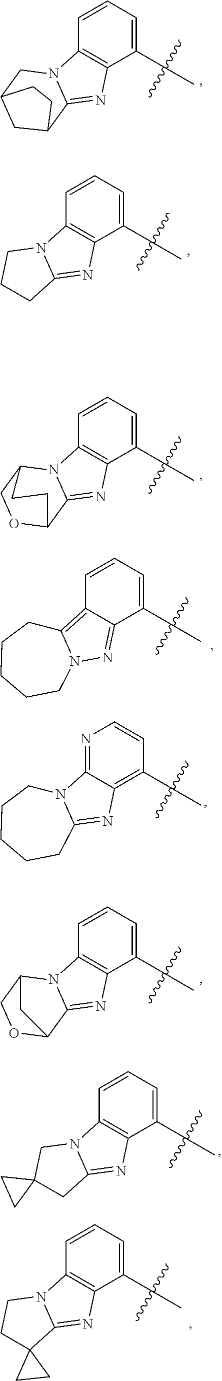

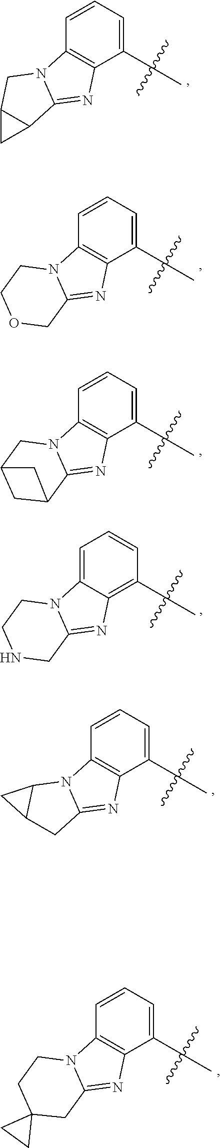 [SMILES: *c1cccc2c1nc1n2CC2CC12 ; *c1cccc2c1nc1n2CCOC1 ; *c1cccc2c1nc1n2CC2CC1C2 ; *c1cccc2c1nc1n2CCNC1 ; *c1cccc2c1nc1n2C2CC2C1 ; *c1cccc2c1nc1n2CCC2(CC2)C1]

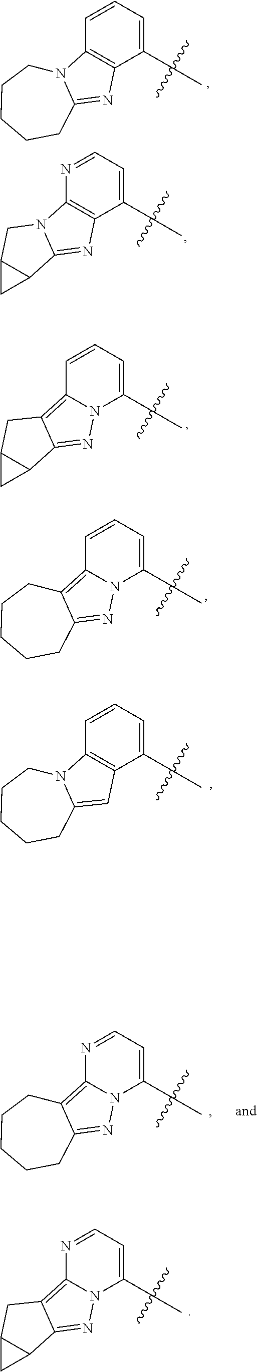

In some embodiments, the structural unit

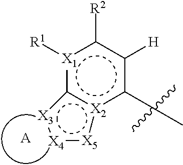

is selected from

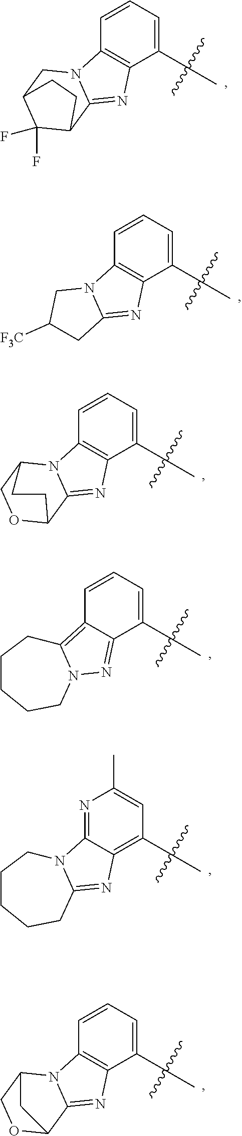

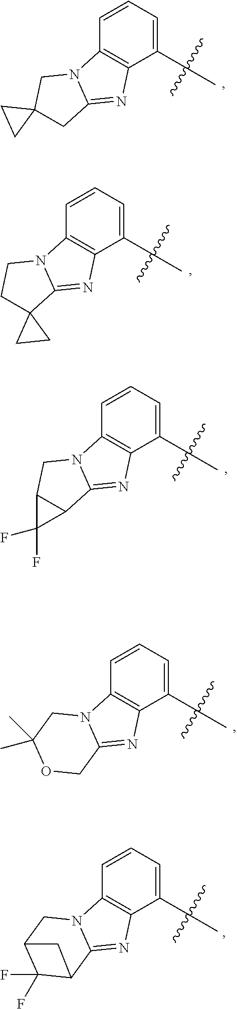

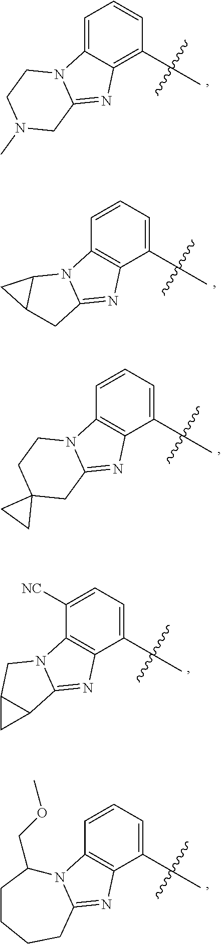

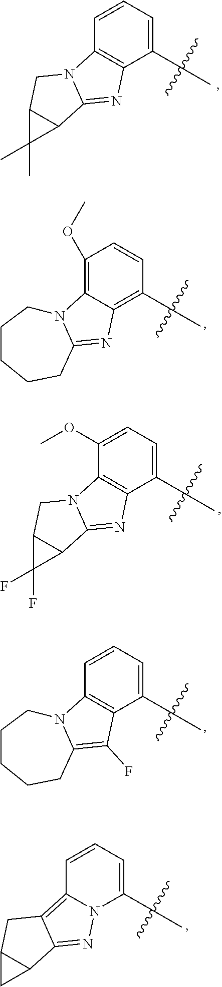

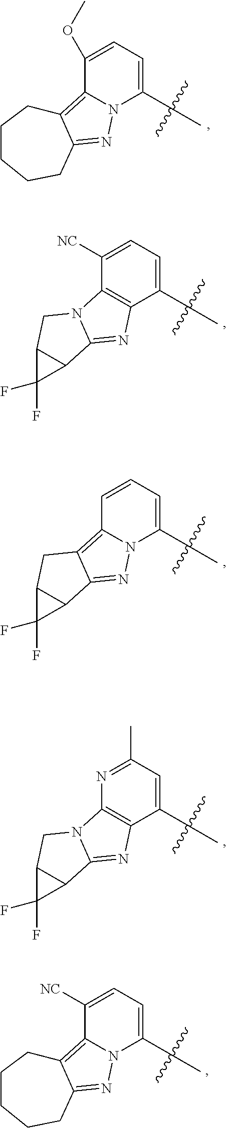

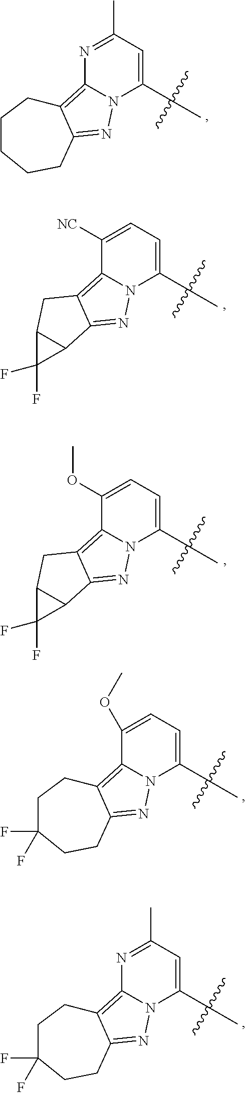

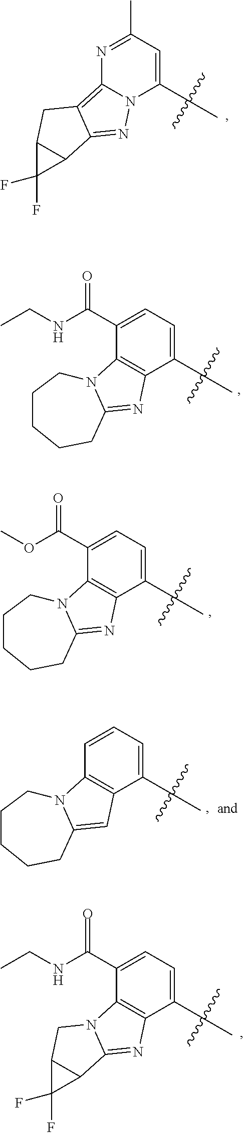

In some embodiments, X₁, X₂, X₃, X₄, and X₅ are each independently CR^{a} or N, wherein R^{a} are each independently selected from being absent, H, and F.

In some embodiments, X₅ is N.

In some embodiments, at least one of X₁, X₂, X₃, X₄, and X₅ is N.

In some embodiments, 1, 2, or 3 of X₁, X₂, X₃, X₄, and X₅ are N.

In some embodiments, X₂ and X₅ are N; in some embodiments, X₃ and X₅ are N; in some embodiments, X₄ and X₅ are N.

In some embodiments, the compound of formula (II) or the pharmaceutically acceptable salt thereof of the present disclosure is selected from a compound of formula (III) or a pharmaceutically acceptable salt thereof:

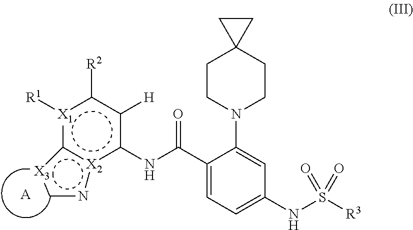

In formula (III):
A is a monocyclic or bicyclic ring selected from 4-10 membered carbon ring and 4-10 membered heterocycle, wherein the 4-10 membered carbon ring or 4-10 membered heterocycle is optionally substituted with one or more R^{b}, and each R is independently selected from halogen, OH, C₁-C₆ alkyl, O(C₁-C₄ alkyl), NH₂, NH(C₁-C₄ alkyl), and N(C₁-C₄ alkyl)₂, wherein the C₁-C₆ alkyl is optionally substituted with one or more groups independently selected from halogen, cyano, OH, NH₂, and O(C₁-C₄ alkyl);
X₁, X₂, and X₃ are each independently C or N;
R¹ is absent, H, C₁-C₆ alkyl, cyano, C₁-C₆ alkoxy, (CH₂)ᵣ—C(O)R^{d}, halogen, (CH₂)ᵣ—C₃-C₁₀ cycloalkyl, (CH₂)ᵣ—C₅-C₁₀ cycloalkenyl, (CH₂)ᵣ-4-10 membered heterocyclyl, (CH₂)ᵣ—C₆-C₁₀ aryl, or (CH₂)ᵣ-5-10 membered heteroaryl, wherein r is 0, 1, 2, or 3, and the C₁-C₆ alkyl, C₁-C₆ alkoxy, C₃-C₁₀ cycloalkyl, C₅-C₁₀ cycloalkenyl, 4-10 membered heterocyclyl, C₆-C₁₀ aryl, or 5-10 membered heteroaryl is optionally substituted with one or more groups independently selected from halogen, CN, OH, NH₂, C₁-C₆ alkyl, C₃-C₆ cycloalkyl, 4-7 membered heterocyclyl, O(C₁-C₄ alkyl), NH(C₁-C₄ alkyl), and N(C₁-C₄ alkyl)₂;
R² is H, C₁-C₆ alkyl, cyano, C₁-C₆ alkoxy, C(O)R^{d}, halogen, C₁-C₆ haloalkyl, or C₁-C₆ haloalkoxy;
R^{d} is selected from OH, C₁-C₆ alkyl, O(C₁-C₄ alkyl), NH₂, NH(C₁-C₄ alkyl), and N(C₁-C₄ alkyl)₂, and when one of R¹ and R² is H, then the other is not H, cyano, or halogen; and
R³ is selected from C₁-C₆ alkyl optionally substituted with one or more halogen or OH.

In some embodiments, R¹ in formula (II) or formula (III) is absent, H, C₁-C₆ alkyl, cyano, C₁-C₆ alkoxy, or C(O)R^{d}.

In some embodiments, R¹ in formula (II) or formula (III) is absent, H, cyano, C₁-C₆ alkoxy, or C(O)R^{d}.

In some embodiments, R¹ in formula (II) or formula (III) is absent, H, cyano, methoxy, C(O)NHCH₂CH₃, or C(O)OCH₃.

In some embodiments, R² in formula (II) or formula (III) is H, C₁-C₆ alkyl, cyano, C₁-C₆ alkoxy, or C(O)R^{d}.

In some embodiments, R² in formula (II) or (III) is H or C₁-C₆ alkyl.

In some embodiments, R² in formula (II) or formula (III) is H or methyl.

In some embodiments, R^{d} in formula (II) or formula (III) is selected from O(C₁-C₄ alkyl), NH₂, NH(C₁-C₄ alkyl), and N(C₁-C₄ alkyl)₂.

In some embodiments, R^{d} in formula (II) or formula (III) is selected from O(C₁-C₄ alkyl) and NH(C₁-C₄ alkyl).

In some embodiments, R^{d} in formula (II) or formula (III) is selected from methoxy and NHCH₂CH₃.

In some embodiments, R³ in formula (II) or formula (III) is selected from C₁-C₄ alkyl optionally substituted with one or more halogen or OH.

In some embodiments, R³ in formula (II) or formula (III) is selected from C₁-C₄ alkyl optionally substituted with one or more OH.

In some embodiments, R³ in the formula (II) or formula (III) is selected from

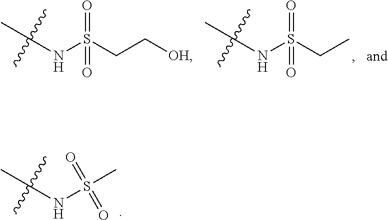

In some embodiments, R³ in formula (II) or formula (III) is selected from

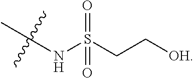 [SMILES: *NS(=O)(=O)CCO]

It should be understood that any embodiment of the compounds of formula (I), formula (II) and formula (III) of the present disclosure as described above and any specific substituents set forth herein with respect to particular ring A, ring M, ring Q, R^{a}, R^{b}, R^{1a}, R^{1b}, R^{2b}, R^{3a}, R^{3b}, X₁ to X⁶, R^{4b}, R¹ to R⁵, R^{x}, R^{xb}, L¹, L², R^{y}, R^{5a}, R^{5b}, X, Y, Z, R^{c}, R^{d}, and other substituents in the compounds of the present disclosure as described above may be independently combined with other embodiments of the present disclosure and/or substituents of the compounds to form embodiments of the present disclosure not specifically set forth above. Further, in the case that a list of substituents is disclosed in the detailed description and/or claims with respect to any particular ring A, ring M, ring Q, R^{a}, R^{1a}, R^{1b}, R^{2b}, R^{3a}, R^{3b}, X¹ to X⁶, R^{4b}, R¹ to R⁵, R^{x}, R^{xb}, L¹, L², R^{y}, R^{5a}, R^{5b}, X, Y, Z, R^{c}, R^{d}, etc., it should be understood that one or more substituents may be deleted from the list and the remaining list of substituents will be considered an embodiment of the present disclosure.

In yet another aspect, the present disclosure also provides the following compounds or pharmaceutically acceptable salts thereof:

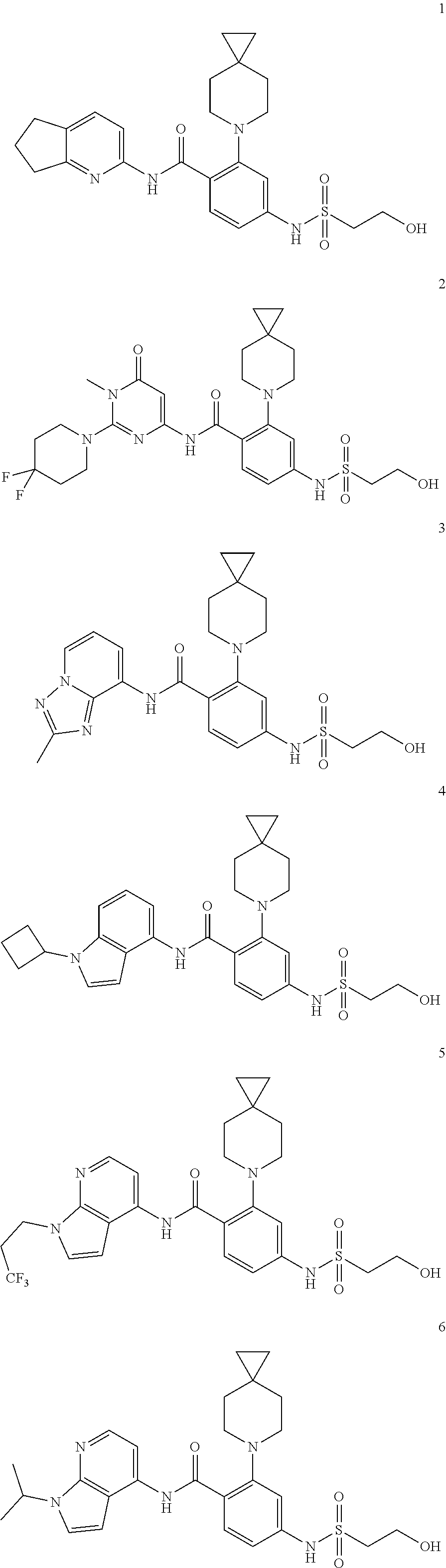 [SMILES: O=C(Nc1ccc2c(n1)CCC2)c1ccc(NS(=O)(=O)CCO)cc1N1CCC2(CC1)CC2 ; Cn1c(N2CCC(F)(F)CC2)nc(NC(=O)c2ccc(NS(=O)(=O)CCO)cc2N2CCC3(CC2)CC3)cc1=O ; Cc1nc2c(NC(=O)c3ccc(NS(=O)(=O)CCO)cc3N3CCC4(CC3)CC4)cccn2n1 ; O=C(Nc1cccc2c1ccn2C1CCC1)c1ccc(NS(=O)(=O)CCO)cc1N1CCC2(CC1)CC2 ; O=C(Nc1ccnc2c1ccn2CCC(F)(F)F)c1ccc(NS(=O)(=O)CCO)cc1N1CCC2(CC1)CC2 ; CC(C)n1ccc2c(NC(=O)c3ccc(NS(=O)(=O)CCO)cc3N3CCC4(CC3)CC4)ccnc21]

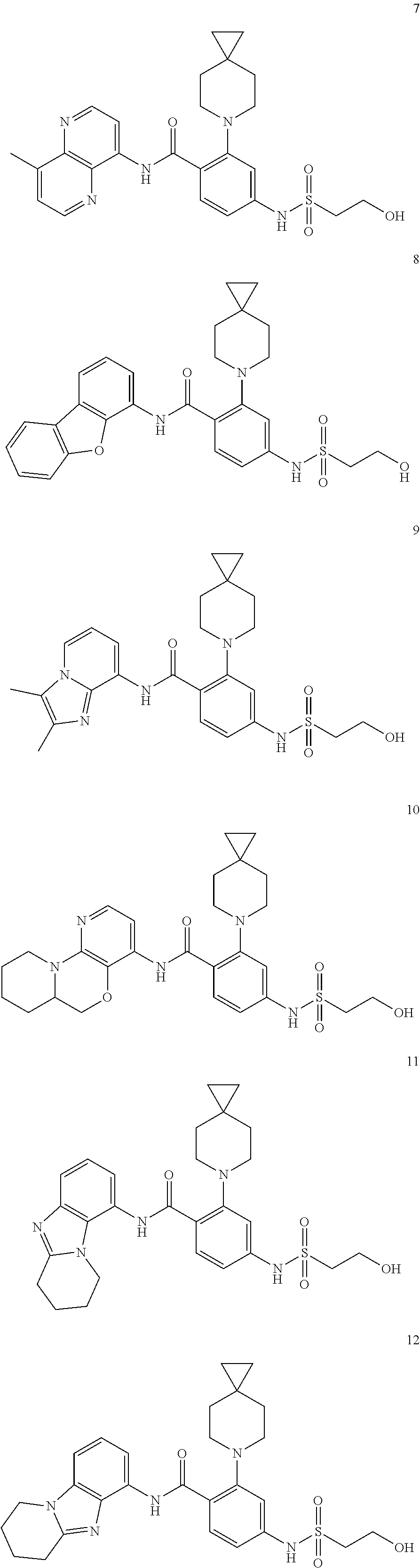 [SMILES: Cc1ccnc2c(NC(=O)c3ccc(NS(=O)(=O)CCO)cc3N3CCC4(CC3)CC4)ccnc12 ; O=C(Nc1cccc2c1oc1ccccc12)c1ccc(NS(=O)(=O)CCO)cc1N1CCC2(CC1)CC2 ; Cc1nc2c(NC(=O)c3ccc(NS(=O)(=O)CCO)cc3N3CCC4(CC3)CC4)cccn2c1C ; O=C(Nc1ccnc2c1OCC1CCCCN21)c1ccc(NS(=O)(=O)CCO)cc1N1CCC2(CC1)CC2 ; O=C(Nc1cccc2nc3n(c12)CCCC3)c1ccc(NS(=O)(=O)CCO)cc1N1CCC2(CC1)CC2 ; O=C(Nc1cccc2c1nc1n2CCCC1)c1ccc(NS(=O)(=O)CCO)cc1N1CCC2(CC1)CC2]

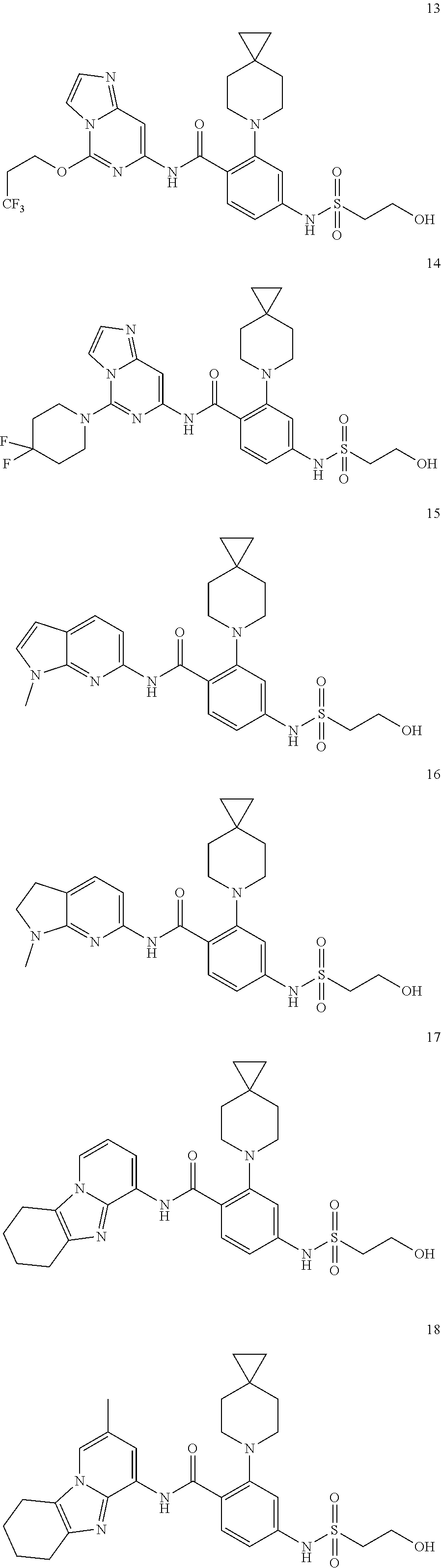 [SMILES: O=C(Nc1cc2nccn2c(OCCC(F)(F)F)n1)c1ccc(NS(=O)(=O)CCO)cc1N1CCC2(CC1)CC2 ; O=C(Nc1cc2nccn2c(N2CCC(F)(F)CC2)n1)c1ccc(NS(=O)(=O)CCO)cc1N1CCC2(CC1)CC2 ; Cn1ccc2ccc(NC(=O)c3ccc(NS(=O)(=O)CCO)cc3N3CCC4(CC3)CC4)nc21 ; CN1CCc2ccc(NC(=O)c3ccc(NS(=O)(=O)CCO)cc3N3CCC4(CC3)CC4)nc21 ; O=C(Nc1cccn2c3c(nc12)CCCC3)c1ccc(NS(=O)(=O)CCO)cc1N1CCC2(CC1)CC2 ; Cc1cc(NC(=O)c2ccc(NS(=O)(=O)CCO)cc2N2CCC3(CC2)CC3)c2nc3c(n2c1)CCCC3]

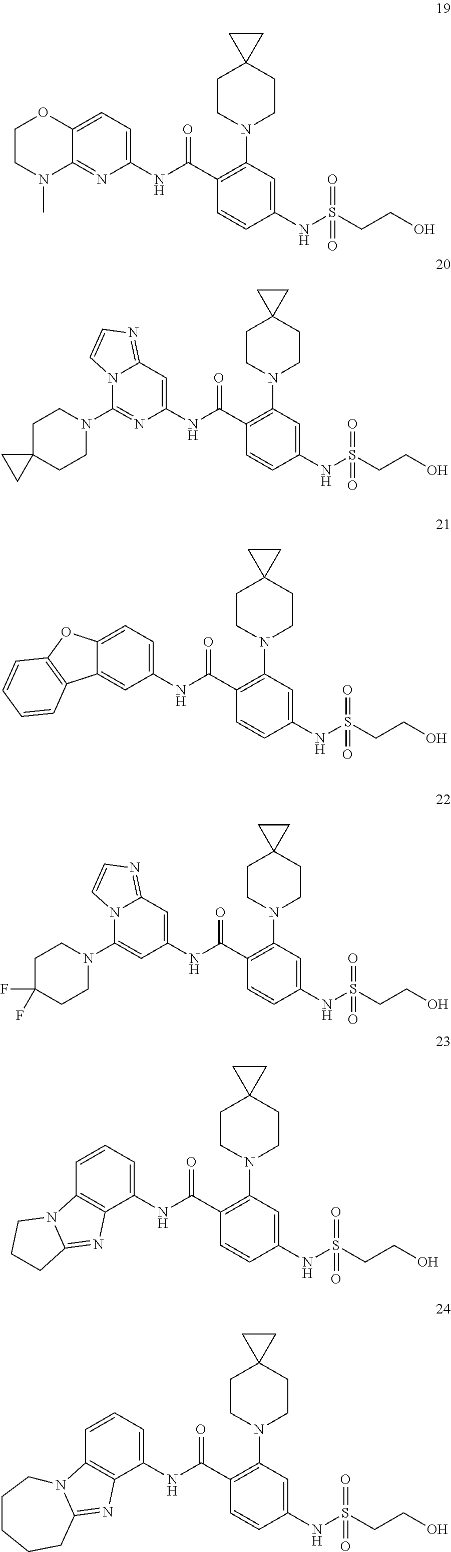 [SMILES: CN1CCOc2ccc(NC(=O)c3ccc(NS(=O)(=O)CCO)cc3N3CCC4(CC3)CC4)nc21 ; O=C(Nc1cc2nccn2c(N2CCC3(CC2)CC3)n1)c1ccc(NS(=O)(=O)CCO)cc1N1CCC2(CC1)CC2 ; O=C(Nc1ccc2oc3ccccc3c2c1)c1ccc(NS(=O)(=O)CCO)cc1N1CCC2(CC1)CC2 ; O=C(Nc1cc(N2CCC(F)(F)CC2)n2ccnc2c1)c1ccc(NS(=O)(=O)CCO)cc1N1CCC2(CC1)CC2 ; O=C(Nc1cccc2c1nc1n2CCC1)c1ccc(NS(=O)(=O)CCO)cc1N1CCC2(CC1)CC2 ; O=C(Nc1cccc2c1nc1n2CCCCC1)c1ccc(NS(=O)(=O)CCO)cc1N1CCC2(CC1)CC2]

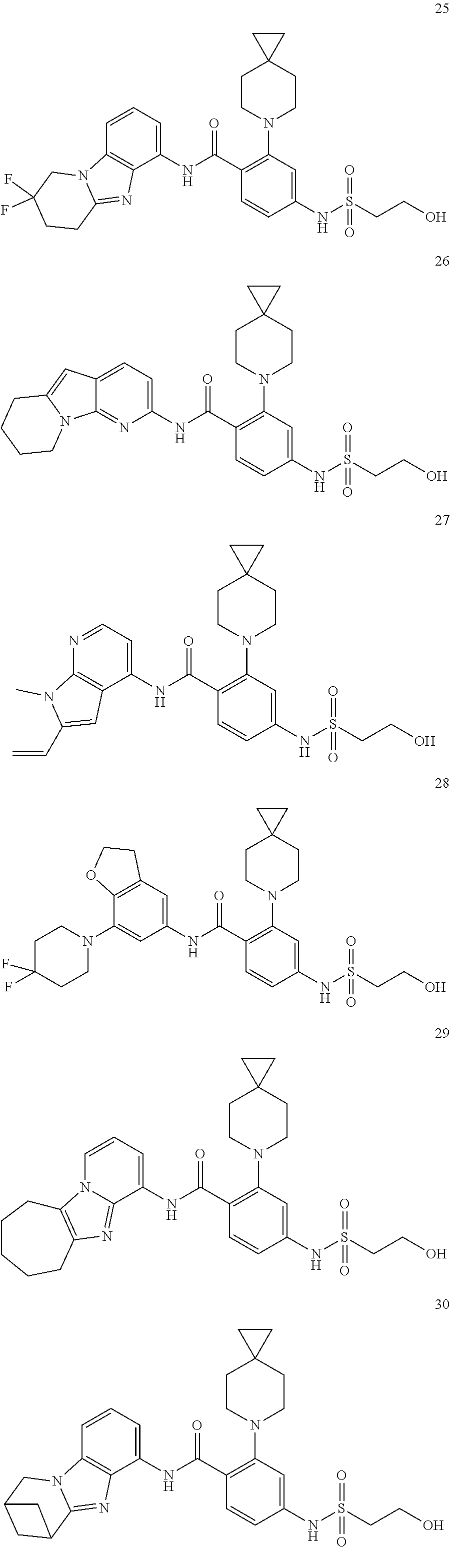 [SMILES: O=C(Nc1cccc2c1nc1n2CC(F)(F)CC1)c1ccc(NS(=O)(=O)CCO)cc1N1CCC2(CC1)CC2 ; O=C(Nc1ccc2cc3n(c2n1)CCCC3)c1ccc(NS(=O)(=O)CCO)cc1N1CCC2(CC1)CC2 ; C=Cc1cc2c(NC(=O)c3ccc(NS(=O)(=O)CCO)cc3N3CCC4(CC3)CC4)ccnc2n1C ; O=C(Nc1cc2c(c(N3CCC(F)(F)CC3)c1)OCC2)c1ccc(NS(=O)(=O)CCO)cc1N1CCC2(CC1)CC2 ; O=C(Nc1cccn2c3c(nc12)CCCCC3)c1ccc(NS(=O)(=O)CCO)cc1N1CCC2(CC1)CC2 ; O=C(Nc1cccc2c1nc1n2CC2CC1C2)c1ccc(NS(=O)(=O)CCO)cc1N1CCC2(CC1)CC2]

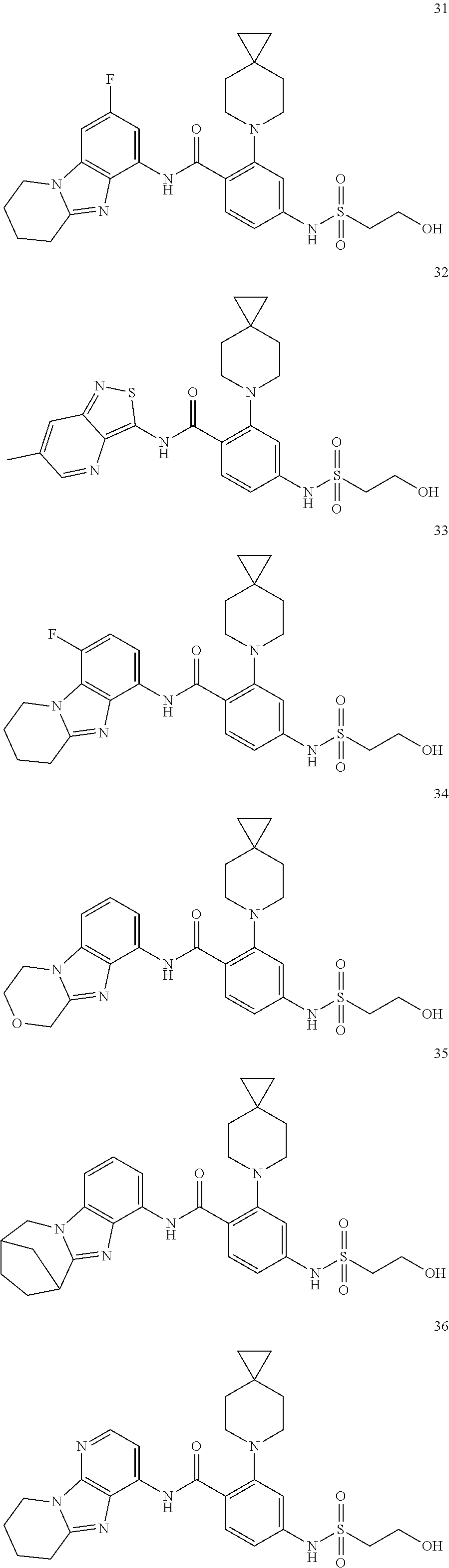 [SMILES: O=C(Nc1cc(F)cc2c1nc1n2CCCC1)c1ccc(NS(=O)(=O)CCO)cc1N1CCC2(CC1)CC2 ; Cc1cnc2c(NC(=O)c3ccc(NS(=O)(=O)CCO)cc3N3CCC4(CC3)CC4)snc2c1 ; O=C(Nc1ccc(F)c2c1nc1n2CCCC1)c1ccc(NS(=O)(=O)CCO)cc1N1CCC2(CC1)CC2 ; O=C(Nc1cccc2c1nc1n2CCOC1)c1ccc(NS(=O)(=O)CCO)cc1N1CCC2(CC1)CC2 ; O=C(Nc1cccc2c1nc1n2CC2CCC1C2)c1ccc(NS(=O)(=O)CCO)cc1N1CCC2(CC1)CC2 ; O=C(Nc1ccnc2c1nc1n2CCCC1)c1ccc(NS(=O)(=O)CCO)cc1N1CCC2(CC1)CC2]

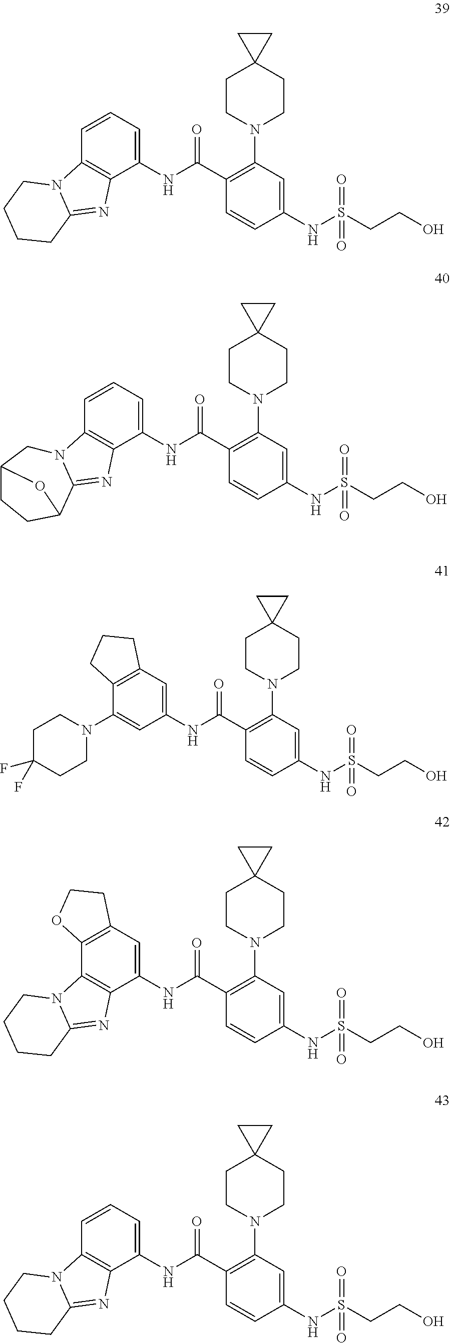 [SMILES: O=C(Nc1cccc2c1nc1n2CCCC1)c1ccc(NS(=O)(=O)CCO)cc1N1CCC2(CC1)CC2 ; O=C(Nc1cccc2c1nc1n2CC2CCC1O2)c1ccc(NS(=O)(=O)CCO)cc1N1CCC2(CC1)CC2 ; O=C(Nc1cc2c(c(N3CCC(F)(F)CC3)c1)CCC2)c1ccc(NS(=O)(=O)CCO)cc1N1CCC2(CC1)CC2 ; O=C(Nc1cc2c(c3c1nc1n3CCCC1)OCC2)c1ccc(NS(=O)(=O)CCO)cc1N1CCC2(CC1)CC2 ; O=C(Nc1cccc2c1nc1n2CCCC1)c1ccc(NS(=O)(=O)CCO)cc1N1CCC2(CC1)CC2]

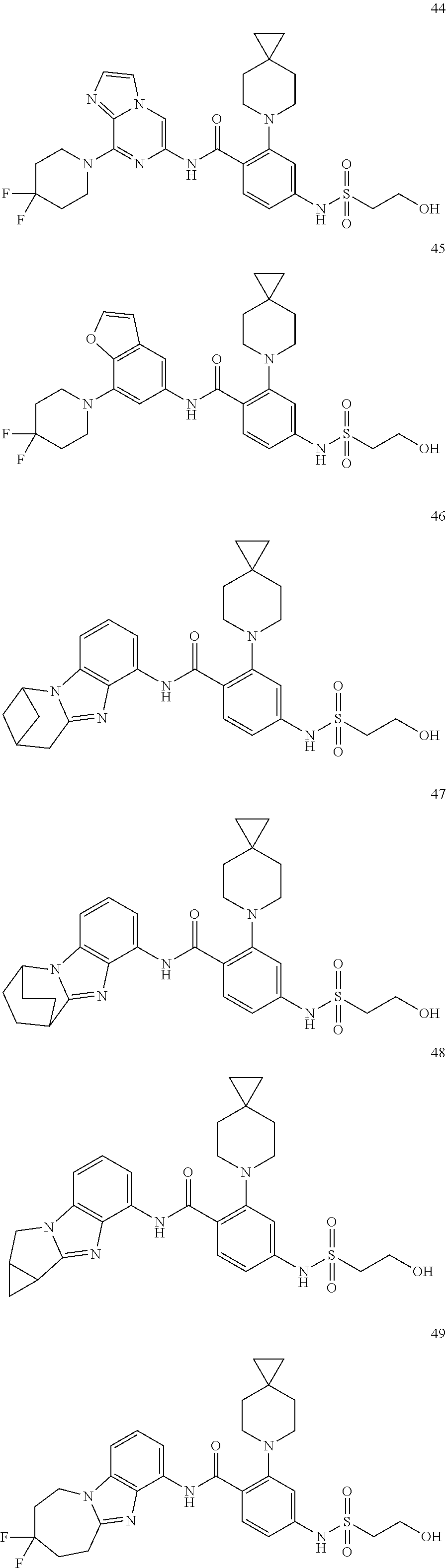 [SMILES: O=C(Nc1cn2ccnc2c(N2CCC(F)(F)CC2)n1)c1ccc(NS(=O)(=O)CCO)cc1N1CCC2(CC1)CC2 ; O=C(Nc1cc(N2CCC(F)(F)CC2)c2occc2c1)c1ccc(NS(=O)(=O)CCO)cc1N1CCC2(CC1)CC2 ; O=C(Nc1cccc2c1nc1n2C2CC(C1)C2)c1ccc(NS(=O)(=O)CCO)cc1N1CCC2(CC1)CC2 ; O=C(Nc1cccc2c1nc1n2C2CCC1CC2)c1ccc(NS(=O)(=O)CCO)cc1N1CCC2(CC1)CC2 ; O=C(Nc1cccc2c1nc1n2CC2CC12)c1ccc(NS(=O)(=O)CCO)cc1N1CCC2(CC1)CC2 ; O=C(Nc1cccc2c1nc1n2CCC(F)(F)CC1)c1ccc(NS(=O)(=O)CCO)cc1N1CCC2(CC1)CC2]

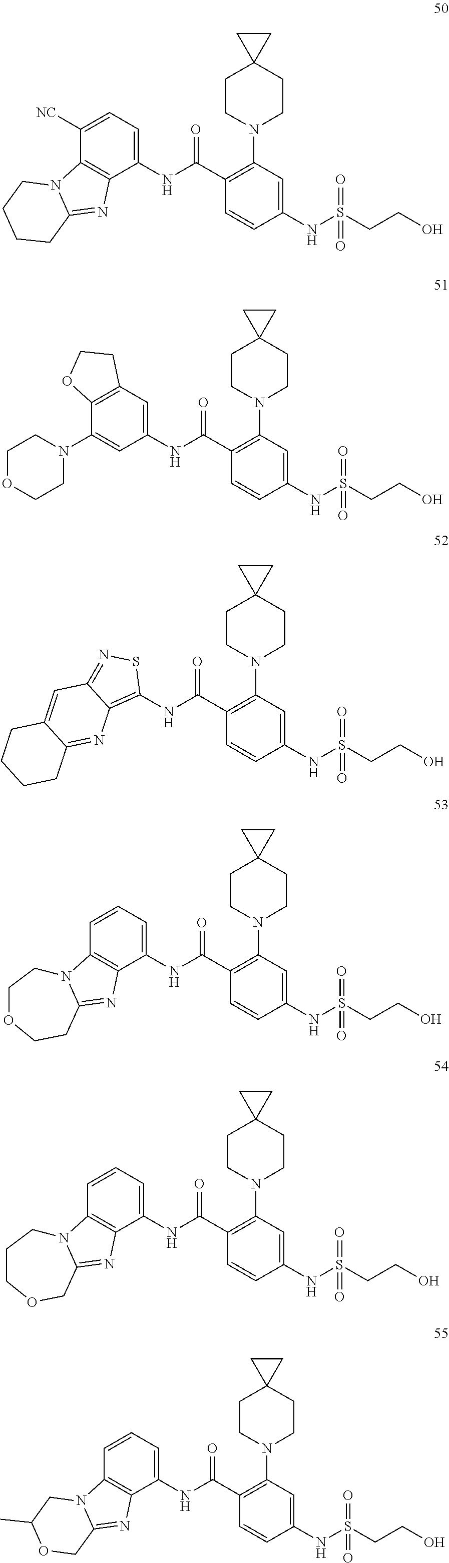 [SMILES: N#Cc1ccc(NC(=O)c2ccc(NS(=O)(=O)CCO)cc2N2CCC3(CC2)CC3)c2nc3n(c12)CCCC3 ; O=C(Nc1cc2c(c(N3CCOCC3)c1)OCC2)c1ccc(NS(=O)(=O)CCO)cc1N1CCC2(CC1)CC2 ; O=C(Nc1snc2cc3c(nc12)CCCC3)c1ccc(NS(=O)(=O)CCO)cc1N1CCC2(CC1)CC2 ; O=C(Nc1cccc2c1nc1n2CCOCC1)c1ccc(NS(=O)(=O)CCO)cc1N1CCC2(CC1)CC2 ; O=C(Nc1cccc2c1nc1n2CCCOC1)c1ccc(NS(=O)(=O)CCO)cc1N1CCC2(CC1)CC2 ; CC1Cn2c(nc3c(NC(=O)c4ccc(NS(=O)(=O)CCO)cc4N4CCC5(CC4)CC5)cccc32)CO1]

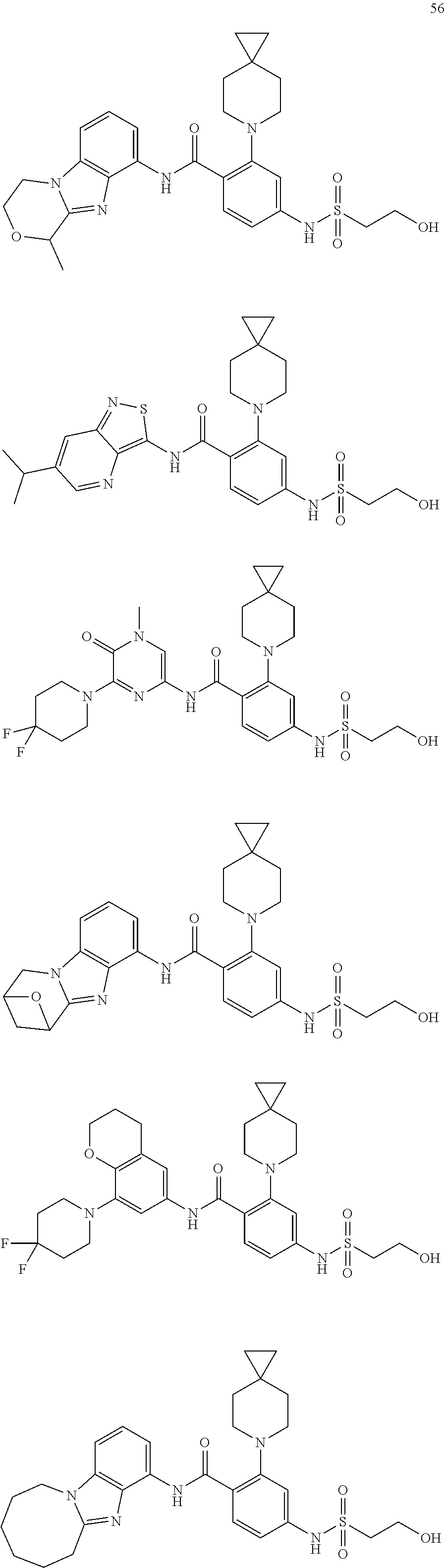 [SMILES: CC1OCCn2c1nc1c(NC(=O)c3ccc(NS(=O)(=O)CCO)cc3N3CCC4(CC3)CC4)cccc12 ; CC(C)c1cnc2c(NC(=O)c3ccc(NS(=O)(=O)CCO)cc3N3CCC4(CC3)CC4)snc2c1 ; Cn1cc(NC(=O)c2ccc(NS(=O)(=O)CCO)cc2N2CCC3(CC2)CC3)nc(N2CCC(F)(F)CC2)c1=O ; O=C(Nc1cccc2c1nc1n2CC2CC1O2)c1ccc(NS(=O)(=O)CCO)cc1N1CCC2(CC1)CC2 ; O=C(Nc1cc2c(c(N3CCC(F)(F)CC3)c1)OCCC2)c1ccc(NS(=O)(=O)CCO)cc1N1CCC2(CC1)CC2 ; O=C(Nc1cccc2c1nc1n2CCCCCC1)c1ccc(NS(=O)(=O)CCO)cc1N1CCC2(CC1)CC2]

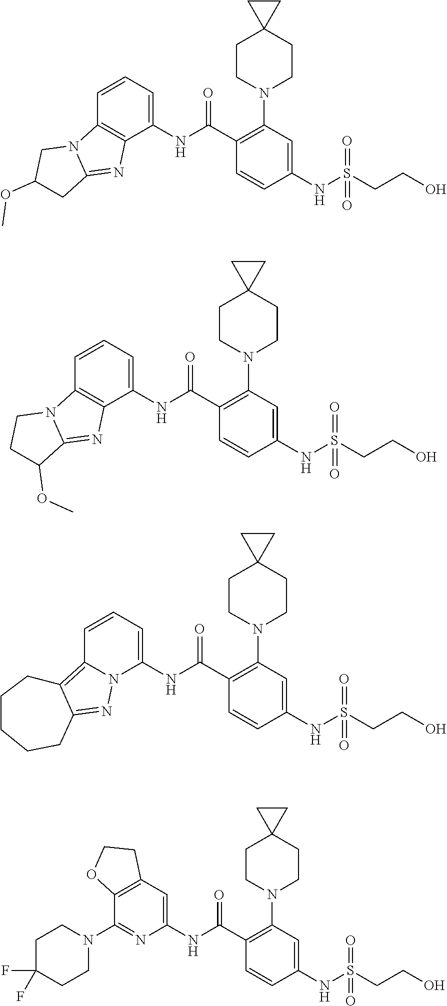 [SMILES: COC1Cc2nc3c(NC(=O)c4ccc(NS(=O)(=O)CCO)cc4N4CCC5(CC4)CC5)cccc3n2C1 ; COC1CCn2c1nc1c(NC(=O)c3ccc(NS(=O)(=O)CCO)cc3N3CCC4(CC3)CC4)cccc12 ; O=C(Nc1cccc2c3c(nn12)CCCCC3)c1ccc(NS(=O)(=O)CCO)cc1N1CCC2(CC1)CC2 ; O=C(Nc1cc2c(c(N3CCC(F)(F)CC3)n1)OCC2)c1ccc(NS(=O)(=O)CCO)cc1N1CCC2(CC1)CC2]

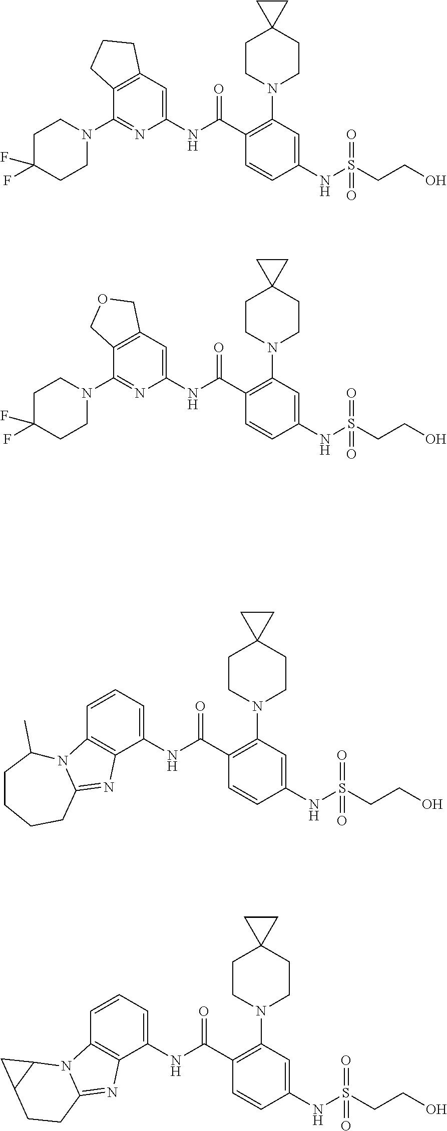 [SMILES: O=C(Nc1cc2c(c(N3CCC(F)(F)CC3)n1)CCC2)c1ccc(NS(=O)(=O)CCO)cc1N1CCC2(CC1)CC2 ; O=C(Nc1cc2c(c(N3CCC(F)(F)CC3)n1)COC2)c1ccc(NS(=O)(=O)CCO)cc1N1CCC2(CC1)CC2 ; CC1CCCCc2nc3c(NC(=O)c4ccc(NS(=O)(=O)CCO)cc4N4CCC5(CC4)CC5)cccc3n21 ; O=C(Nc1cccc2c1nc1n2C2CC2CC1)c1ccc(NS(=O)(=O)CCO)cc1N1CCC2(CC1)CC2]

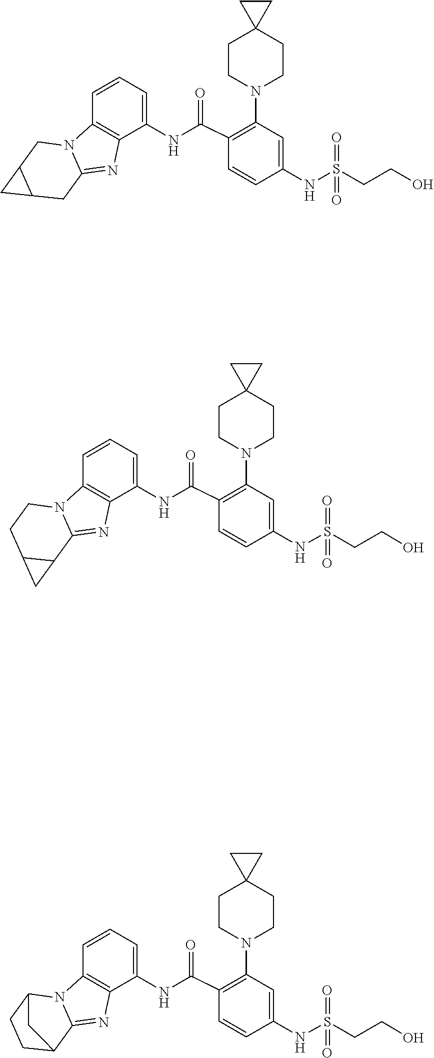 [SMILES: O=C(Nc1cccc2c1nc1n2CC2CC2C1)c1ccc(NS(=O)(=O)CCO)cc1N1CCC2(CC1)CC2 ; O=C(Nc1cccc2c1nc1n2CCC2CC12)c1ccc(NS(=O)(=O)CCO)cc1N1CCC2(CC1)CC2 ; O=C(Nc1cccc2c1nc1n2C2CCC1C2)c1ccc(NS(=O)(=O)CCO)cc1N1CCC2(CC1)CC2]

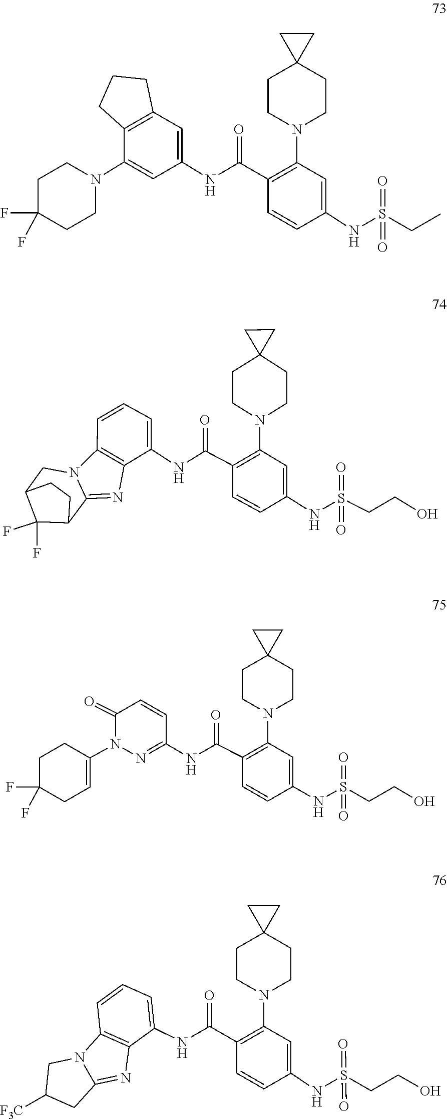 [SMILES: CCS(=O)(=O)Nc1ccc(C(=O)Nc2cc3c(c(N4CCC(F)(F)CC4)c2)CCC3)c(N2CCC3(CC2)CC3)c1 ; O=C(Nc1cccc2c1nc1n2CC2CCC1C2(F)F)c1ccc(NS(=O)(=O)CCO)cc1N1CCC2(CC1)CC2 ; O=C(Nc1ccc(=O)n(C2=CCC(F)(F)CC2)n1)c1ccc(NS(=O)(=O)CCO)cc1N1CCC2(CC1)CC2 ; O=C(Nc1cccc2c1nc1n2CC(C(F)(F)F)C1)c1ccc(NS(=O)(=O)CCO)cc1N1CCC2(CC1)CC2]

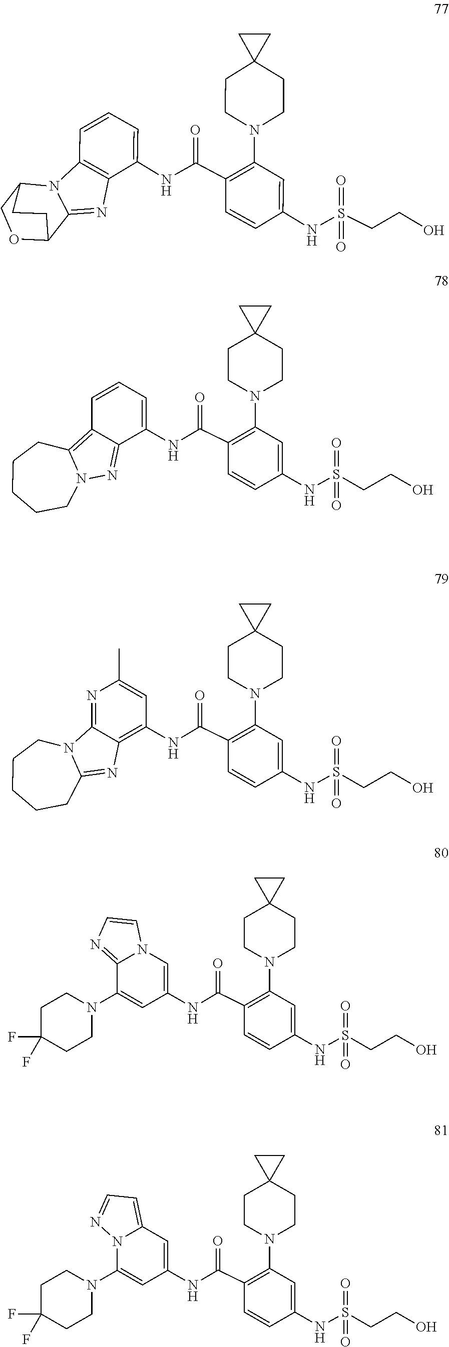 [SMILES: O=C(Nc1cccc2c1nc1n2C2CCC1OC2)c1ccc(NS(=O)(=O)CCO)cc1N1CCC2(CC1)CC2 ; O=C(Nc1cccc2c3n(nc12)CCCCC3)c1ccc(NS(=O)(=O)CCO)cc1N1CCC2(CC1)CC2 ; Cc1cc(NC(=O)c2ccc(NS(=O)(=O)CCO)cc2N2CCC3(CC2)CC3)c2nc3n(c2n1)CCCCC3 ; O=C(Nc1cc(N2CCC(F)(F)CC2)c2nccn2c1)c1ccc(NS(=O)(=O)CCO)cc1N1CCC2(CC1)CC2 ; O=C(Nc1cc(N2CCC(F)(F)CC2)n2nccc2c1)c1ccc(NS(=O)(=O)CCO)cc1N1CCC2(CC1)CC2]

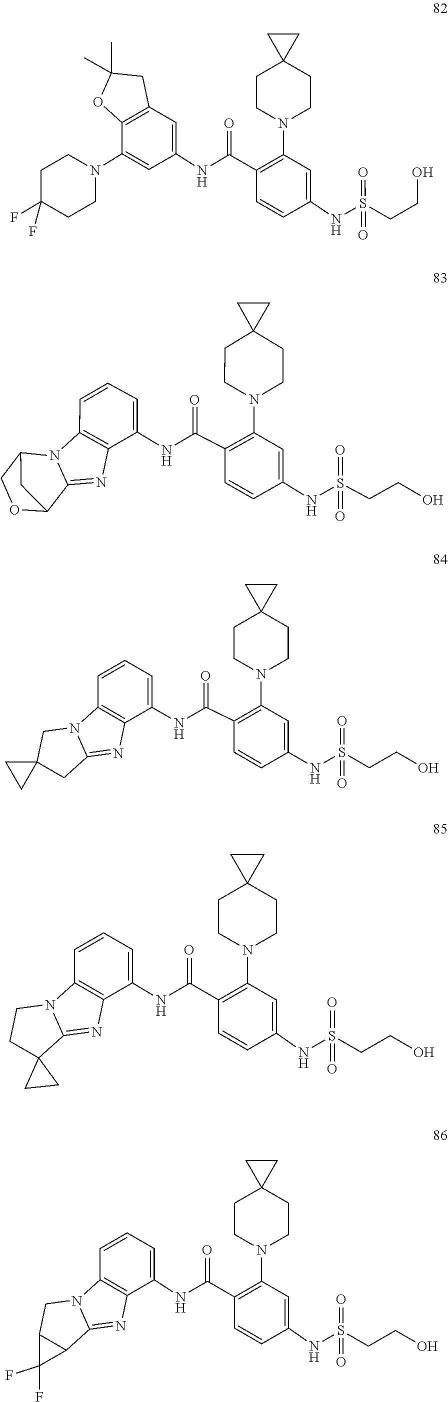 [SMILES: CC1(C)Cc2cc(NC(=O)c3ccc(NS(=O)(=O)CCO)cc3N3CCC4(CC3)CC4)cc(N3CCC(F)(F)CC3)c2O1 ; O=C(Nc1cccc2c1nc1n2C2COC1C2)c1ccc(NS(=O)(=O)CCO)cc1N1CCC2(CC1)CC2 ; O=C(Nc1cccc2c1nc1n2CC2(CC2)C1)c1ccc(NS(=O)(=O)CCO)cc1N1CCC2(CC1)CC2 ; O=C(Nc1cccc2c1nc1n2CCC12CC2)c1ccc(NS(=O)(=O)CCO)cc1N1CCC2(CC1)CC2 ; O=C(Nc1cccc2c1nc1n2CC2C1C2(F)F)c1ccc(NS(=O)(=O)CCO)cc1N1CCC2(CC1)CC2]

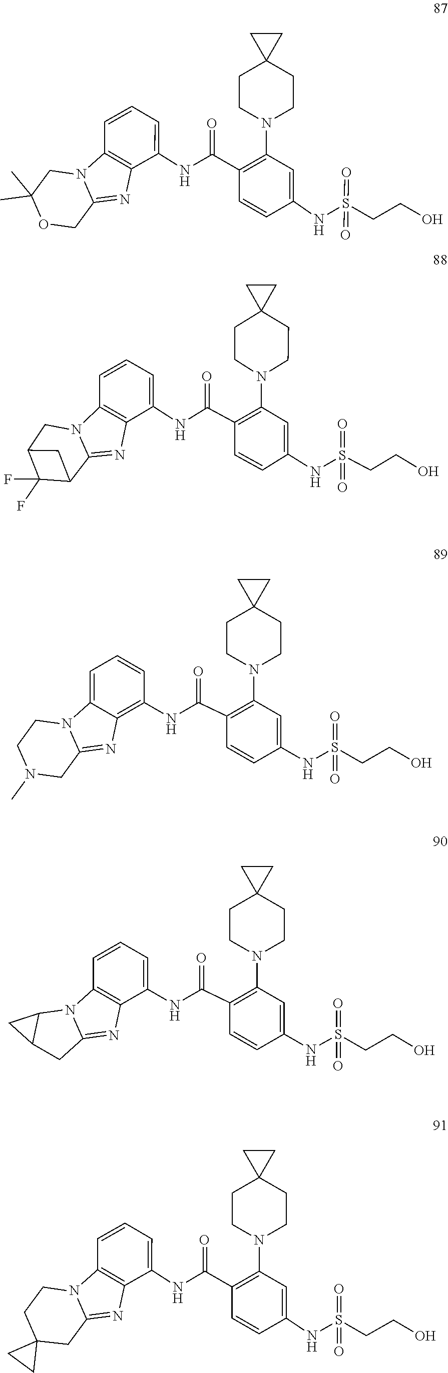

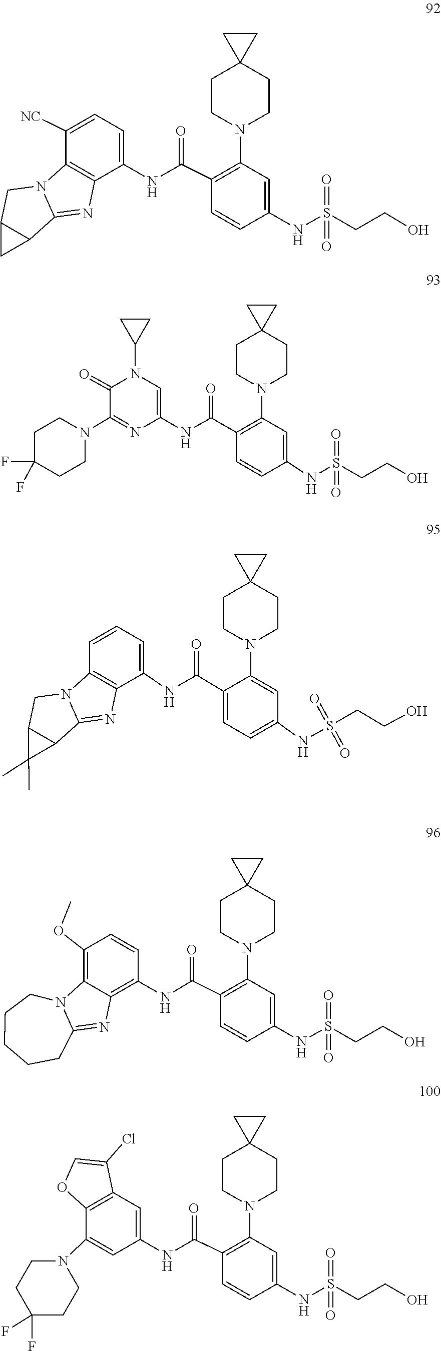 [SMILES: N#Cc1ccc(NC(=O)c2ccc(NS(=O)(=O)CCO)cc2N2CCC3(CC2)CC3)c2nc3n(c12)CC1CC31 ; O=C(Nc1cn(C2CC2)c(=O)c(N2CCC(F)(F)CC2)n1)c1ccc(NS(=O)(=O)CCO)cc1N1CCC2(CC1)CC2 ; CC1(C)C2Cn3c(nc4c(NC(=O)c5ccc(NS(=O)(=O)CCO)cc5N5CCC6(CC5)CC6)cccc43)C21 ; COc1ccc(NC(=O)c2ccc(NS(=O)(=O)CCO)cc2N2CCC3(CC2)CC3)c2nc3n(c12)CCCCC3 ; O=C(Nc1cc(N2CCC(F)(F)CC2)c2occ(Cl)c2c1)c1ccc(NS(=O)(=O)CCO)cc1N1CCC2(CC1)CC2]

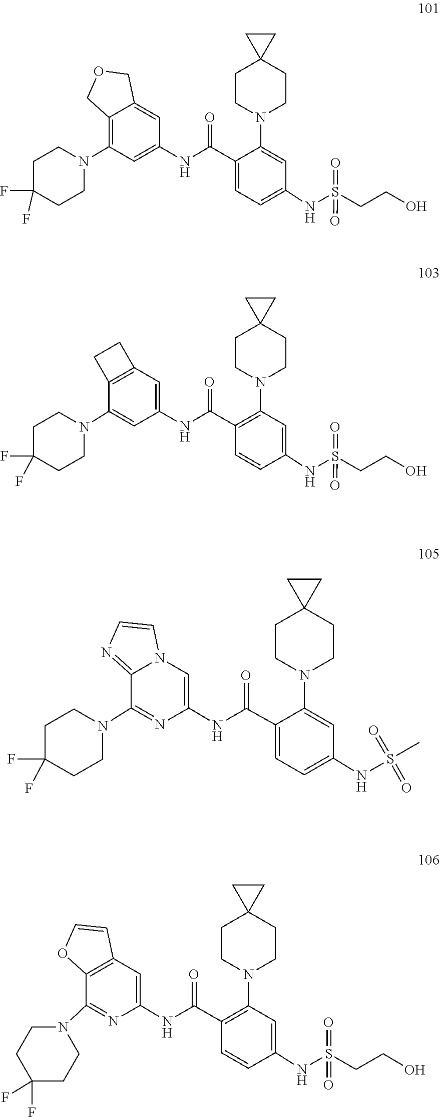 [SMILES: O=C(Nc1cc2c(c(N3CCC(F)(F)CC3)c1)COC2)c1ccc(NS(=O)(=O)CCO)cc1N1CCC2(CC1)CC2 ; O=C(Nc1cc2c(c(N3CCC(F)(F)CC3)c1)CC2)c1ccc(NS(=O)(=O)CCO)cc1N1CCC2(CC1)CC2 ; CS(=O)(=O)Nc1ccc(C(=O)Nc2cn3ccnc3c(N3CCC(F)(F)CC3)n2)c(N2CCC3(CC2)CC3)c1 ; O=C(Nc1cc2ccoc2c(N2CCC(F)(F)CC2)n1)c1ccc(NS(=O)(=O)CCO)cc1N1CCC2(CC1)CC2]

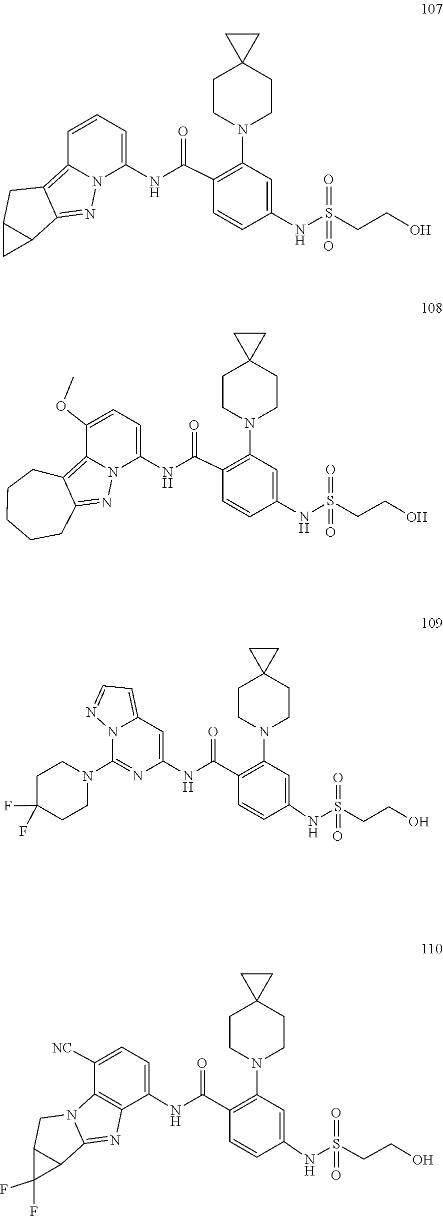 [SMILES: O=C(Nc1cccc2c3c(nn12)C1CC1C3)c1ccc(NS(=O)(=O)CCO)cc1N1CCC2(CC1)CC2 ; COc1ccc(NC(=O)c2ccc(NS(=O)(=O)CCO)cc2N2CCC3(CC2)CC3)n2nc3c(c12)CCCCC3 ; O=C(Nc1cc2ccnn2c(N2CCC(F)(F)CC2)n1)c1ccc(NS(=O)(=O)CCO)cc1N1CCC2(CC1)CC2 ; N#Cc1ccc(NC(=O)c2ccc(NS(=O)(=O)CCO)cc2N2CCC3(CC2)CC3)c2nc3n(c12)CC1C3C1(F)F]

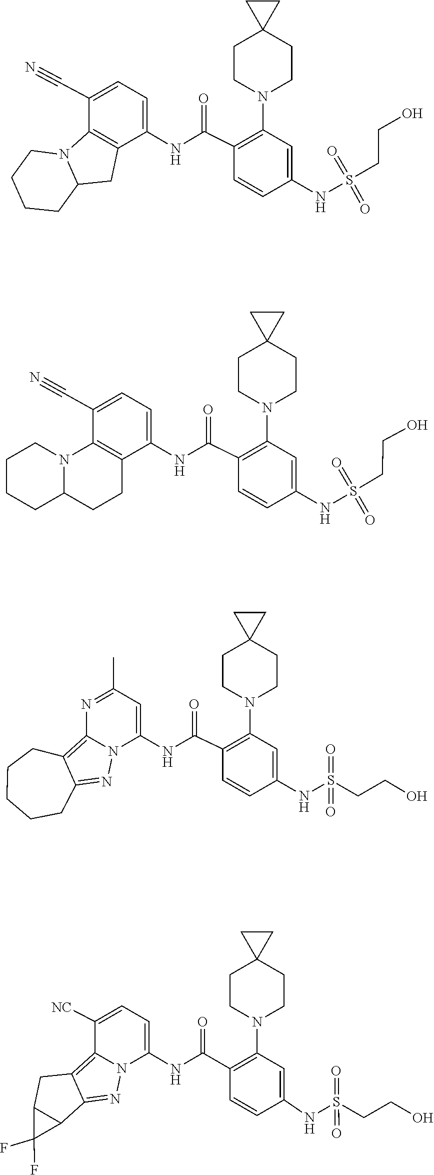 [SMILES: N#Cc1ccc(NC(=O)c2ccc(NS(=O)(=O)CCO)cc2N2CCC3(CC2)CC3)c2c1N1CCCCC1C2 ; N#Cc1ccc(NC(=O)c2ccc(NS(=O)(=O)CCO)cc2N2CCC3(CC2)CC3)c2c1N1CCCCC1CC2 ; Cc1cc(NC(=O)c2ccc(NS(=O)(=O)CCO)cc2N2CCC3(CC2)CC3)n2nc3c(c2n1)CCCCC3 ; N#Cc1ccc(NC(=O)c2ccc(NS(=O)(=O)CCO)cc2N2CCC3(CC2)CC3)n2nc3c(c12)CC1C3C1(F)F]

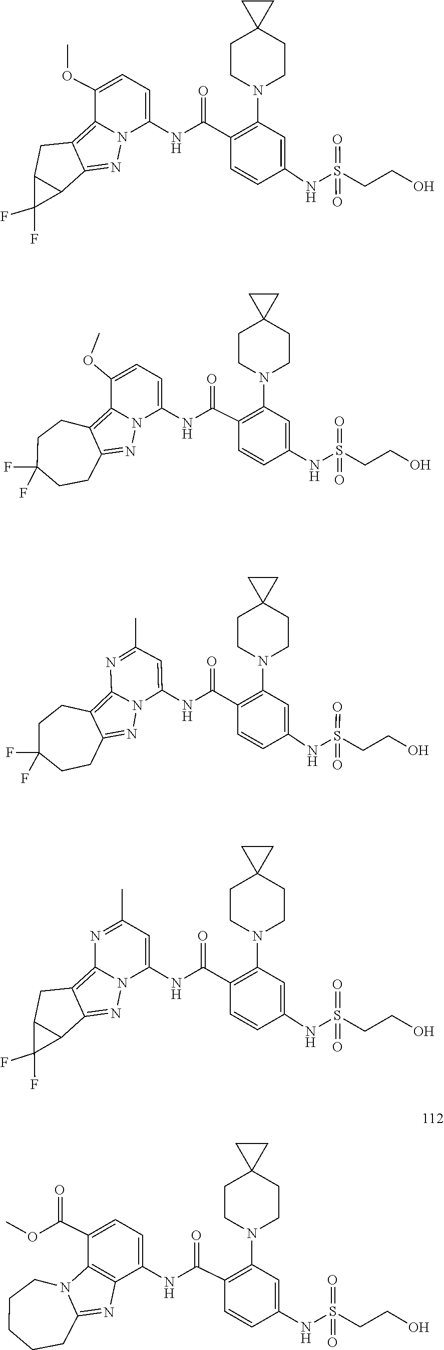 [SMILES: COc1ccc(NC(=O)c2ccc(NS(=O)(=O)CCO)cc2N2CCC3(CC2)CC3)n2nc3c(c12)CC1C3C1(F)F ; COc1ccc(NC(=O)c2ccc(NS(=O)(=O)CCO)cc2N2CCC3(CC2)CC3)n2nc3c(c12)CCC(F)(F)CC3 ; Cc1cc(NC(=O)c2ccc(NS(=O)(=O)CCO)cc2N2CCC3(CC2)CC3)n2nc3c(c2n1)CCC(F)(F)CC3 ; Cc1cc(NC(=O)c2ccc(NS(=O)(=O)CCO)cc2N2CCC3(CC2)CC3)n2nc3c(c2n1)CC1C3C1(F)F ; COC(=O)c1ccc(NC(=O)c2ccc(NS(=O)(=O)CCO)cc2N2CCC3(CC2)CC3)c2nc3n(c12)CCCCC3]

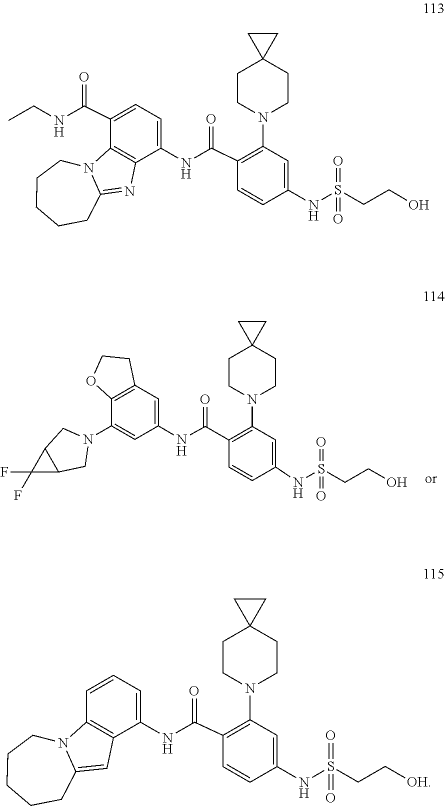 [SMILES: CCNC(=O)c1ccc(NC(=O)c2ccc(NS(=O)(=O)CCO)cc2N2CCC3(CC2)CC3)c2nc3n(c12)CCCCC3 ; O=C(Nc1cc2c(c(N3CC4C(C3)C4(F)F)c1)OCC2)c1ccc(NS(=O)(=O)CCO)cc1N1CCC2(CC1)CC2 ; O=C(Nc1cccc2c1cc1n2CCCCC1)c1ccc(NS(=O)(=O)CCO)cc1N1CCC2(CC1)CC2]

The present disclosure also provides a pharmaceutical composition comprising the compound of formula (I) or the pharmaceutically acceptable salt thereof and a pharmaceutically acceptable excipient.

Further, the present disclosure relates to use of the compound of formula (I) or the pharmaceutically acceptable salt thereof or the pharmaceutical composition thereof in the preparation of a medicament for preventing or treating a KIF18A-related disease.

Further, the present disclosure relates to use of the compound of formula (I) or the pharmaceutically acceptable salt thereof or the pharmaceutical composition thereof in the preparation of a medicament for preventing or treating tumors.

Further, the present disclosure relates to use of the compound of formula (I) or the pharmaceutically acceptable salt thereof or the pharmaceutical composition thereof in preventing or treating a KIF18A-related disease.

Further, the present disclosure relates to use of the compound of formula (I) or the pharmaceutically acceptable salt thereof or the pharmaceutical composition thereof in preventing or treating tumors.

Further, the present disclosure relates to the compound of formula (I) or the pharmaceutically acceptable salt thereof or the pharmaceutical composition thereof for use in preventing or treating a KIF18A-related disease.

Further, the present disclosure relates to the compound of formula (I) or the pharmaceutically acceptable salt thereof or the pharmaceutical composition thereof for use in preventing or treating tumors.

The present disclosure also relates to a method for treating a KIF18A-related disease, which comprises administering to a patient a therapeutically effective amount of the compound of formula (I) or the pharmaceutically acceptable salt thereof or the pharmaceutical composition thereof described in the present disclosure or a pharmaceutical formulation comprising the compound of formula (I) or the pharmaceutically acceptable salt thereof described in the present disclosure.

The present disclosure also relates to a method for treating tumors, which comprises administering to a patient a therapeutically effective amount of the compound of formula (I) or the pharmaceutically acceptable salt thereof or the pharmaceutical composition thereof described in the present disclosure or a pharmaceutical formulation comprising the compound of formula (I) or the pharmaceutically acceptable salt thereof described in the present disclosure.

In some embodiments, the KIF18A-related disease is selected from tumors.

In some embodiments, the KIF18A-related disease is selected from ovarian cancer.

### DEFINITIONS AND DESCRIPTION

Unless otherwise stated, the terms used in the present disclosure have the following meanings, and the definitions of groups and terms described in the present disclosure, including definitions thereof as examples, exemplary definitions, preferred definitions, definitions documented in tables, definitions of specific compounds in the examples, and the like, may be arbitrarily combined and incorporated with each other. A certain term, unless otherwise specifically defined, should not be considered indefinite or unclear, but should be understood according to its common meaning in the field. When referring to a trade name, it is intended to refer to its corresponding commercial product or its active ingredient.

Herein, [symbol] represents a connection site.

Dashed circles within rings of structural units such as

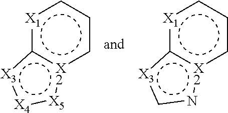

indicate aromatic delocalized bonds.

The term “tautomer” refers to functional isomers resulting from the rapid movement of an atom in a molecule between two positions. The compounds of the present disclosure may exhibit tautomerism. Tautomeric compounds may exist in two or more interconvertible forms. Tautomers generally exist in an equilibrium form. Trying to separate a single tautomer usually leads to a mixture, the physicochemical properties of which are consistent with the mixture of the compound. The position of the equilibrium depends on the chemical properties of the molecule. For example, in many aliphatic aldehydes and ketones such as acetaldehyde, the keto form predominates; whereas in phenol, the enol form predominates. In the present disclosure, all tautomeric forms of the compound are included.

The term “stereoisomer” refers to isomers resulting from different spatial arrangements of atoms in a molecule, including cis-trans isomers, enantiomers, and diastereoisomers.

The compound of the present disclosure may have an asymmetric atom such as a carbon atom, a sulfur atom, a nitrogen atom, and a phosphorus atom, or an asymmetric double bond, and thus the compound of the present disclosure may exist in the form of a particular geometric isomer or stereoisomer. The form of a particular geometric isomer or stereoisomer may be cis and trans isomers, E and Z geometric isomers, (−)- and (+)-enantiomers, (R)- and (S)-enantiomers, diastereoisomers, (D)-isomers, (L)-isomers, and racemic mixtures or other mixtures thereof, such as an enantiomer or diastereoisomer enriched mixture, and all of the above isomers, as well as mixtures thereof, are encompassed within the definition scope of the compound of the present disclosure. An additional asymmetric carbon atom, asymmetric sulfur atom, asymmetric nitrogen atom, or asymmetric phosphorus atom may be present in substituents such as alkyl. All of these isomers and mixtures thereof involved in the substituents are also encompassed within the definition scope of the compound of the present disclosure. The compound with asymmetric atoms of the present disclosure can be separated in an optically active pure form or in a racemic form. The optically active pure form can be obtained by resolving a racemic mixture or by synthesis using chiral starting materials or chiral reagents.

The term “substituted” means that any one or more (e.g., 1, 2, 3, 4, 5, or 6) hydrogen atoms on a specific atom are substituted with substituents, as long as the valence of the specific atom is normal and the compound resulting from the substitution is stable. When the substituent is oxo (namely ═O), it means that two hydrogen atoms are substituted, and oxo is not possible on an aromatic group.

The term “optional” or “optionally” means that the subsequently described event or circumstance may, but not necessarily, occur. The description includes instances where the event or circumstance occurs and instances where the event or circumstance does not. For example, ethyl being “optionally” substituted with one or more halogen means that the ethyl may be unsubstituted (CH₂CH₃), monosubstituted (CH₂CH₂F, CH₂CH₂Cl, or the like), polysubstituted (CHFCH₂F, CH₂CHF₂, CHFCH₂Cl, CH₂CHCl₂, or the like), or fully substituted (CF₂CF₃, CF₂CCl₃, CCl₂CCl₃, or the like). It will be understood by those skilled in the art that for any group comprising one or more substituents, no substitution or substituting pattern that is spatially impossible and/or cannot be synthesized will be introduced.

When any variable (e.g., R^{a} or R^{b}) occurs more than once in the constitution or structure of a compound, the variable is independently defined in each case. For example, if a group is substituted with 2R^{b}, the definition of each R^{b} is independent.

When the number of a linking group is 0, such as —(CH₂)₀—, it means that the linking group is a bond.

When one of variables is selected from a chemical bond or is absent, it means that the two groups which it links are linked directly. For example, when L in A-L-Z represents a bond, it means that the structure is actually A-Z.

When the linking direction of the linking group referred to herein is not specified, the linking direction is arbitrary. For example, when L¹ in the structural unit L¹-L²-R^{y} is selected from “—NR⁵SO₂—”, L¹ may be linked to L²-R^{y} in the left-to-right direction to form “—NR⁵SO₂-L²-R^{y}” or linked to L²-R^{y} in the right-to-left direction to form “—SO₂NR⁵-L²-R^{y}”.

When a chemical bond is cross-linked to two atoms on a ring, it means that the chemical bond can be bonded to any atom on the ring. For example, the structural unit

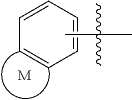

indicates that the chemical bond can be bonded to any position on the benzene ring.

Cₘ-Cₙ used herein means that the moiety has an integer number of carbon atoms in the range of m-n. For example, “C₁-C₁₀” means that the group may have 1 carbon atom, 2 carbon atoms, 3 carbon atoms, 4 carbon atoms, 5 carbon atoms, 6 carbon atoms, 7 carbon atoms, 8 carbon atoms, 9 carbon atoms, or 10 carbon atoms.

The term “azapyridinonyl” refers to groups resulting from the replacement of a ring C atom on pyridinonyl

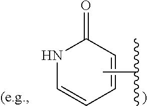

with 1 or 2 N atoms (including, but not limited to,

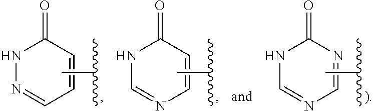

The term “alkyl” refers to a hydrocarbon group with a general formula of CₙH₂ₙ₊₁. The alkyl may be linear or branched. The term “C₁₋₁₀ alkyl” may be understood to represent a linear or branched saturated hydrocarbon group having 1, 2, 3, 4, 5, 6, 7, 8, 9, or 10 carbon atoms. Specific examples of the alkyl include, but are not limited to, methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, 1,2-dimethylbutyl, and the like. The term “C₁-C₆ alkyl” may be understood to represent alkyl having 1 to 6 carbon atoms; specific examples include, but are not limited to, methyl, ethyl, n-propyl, isopropyl, n-butyl, isobutyl, sec-butyl, tert-butyl, n-pentyl, 1-methylbutyl, 2-methylbutyl, 3-methylbutyl, neopentyl, hexyl, 2-methylpentyl, and the like. The term “C₁-C₄ alkyl” may be understood to represent a linear or branched saturated alkyl group having 1 to 4 carbon atoms. The “C₁-C₁₀ alkyl” may include the range of “C₁-C₆ alkyl”, “C₁-C₄ alkyl”, or the like, and the “C₁-C₆ alkyl” may further include “C₁-C₄ alkyl”.

The term “alkylene” may be understood to represent a linear or branched saturated divalent hydrocarbon group. The term “C₁-C₄ alkylene” is understood to represent a linear or branched saturated divalent hydrocarbon group having 1, 2, 3, or 4 carbon atoms. The “C₁-C₄ alkylene” may include “C₁-C₃ alkylene”.

The term “alkoxy” refers to a group derived from a linear or branched alcohol by loss of a hydrogen atom from hydroxy, and may be understood as “alkyloxy” or “alkyl-O—”. The term “C₁-C₁₀ alkoxy” may be understood as “C₁-C₁₀ alkyloxy” or “C₁-C₁₀ alkyl-O—”; the term “C₁-C₆ alkoxy” may be understood as “C₁-C₆ alkyloxy” or “C₁-C₆ alkyl-O—”. The “C₁-C₁₀ alkoxy” may include the ranges of “C₁-C₆ alkoxy”, “C₁-C₃ alkoxy”, and the like, and the “C₁-C₆ alkoxy” may further include “C₁-C₃ alkoxy”.

The term “haloalkyl” means that one, two, or more hydrogen atoms or all hydrogen atoms in the alkyl as defined above are substituted with halogen. “Haloalkyl” may be C₁-C₈ haloalkyl, C₁-C₆ haloalkyl, or C₁-C₄ haloalkyl. The term “haloalkoxy” means that one, two, or more hydrogen atoms or all hydrogen atoms in the alkoxy as defined above are substituted with halogen. “Haloalkoxy” may be C₁-C₈ haloalkoxy, C₁-C₆ haloalkoxy, or C₁-C₄ haloalkoxy.

The term “alkenyl” refers to a linear or branched unsaturated aliphatic hydrocarbon group consisting of carbon atoms and hydrogen atoms and having at least one double bond. The term “C₂-C₁₀ alkenyl” may be understood to represent a linear or branched unsaturated hydrocarbon group comprising one or more double bonds and having 2, 3, 4, 5, 6, 7, 8, 9, or 10 carbon atoms, and the “C₂-C₁₀ alkenyl” may include “C₂-C₆ alkenyl”, “C₂-C₄ alkenyl”, and C₂ or C₃ alkenyl. It can be understood that in the case that the alkenyl comprises more than one double bond, the double bonds can be separated from one another or conjugated. Specific examples of alkenyl include, but are not limited to, vinyl, allyl, (E)-2-methylvinyl, (Z)-2-methylvinyl, (E)-but-2-enyl, (Z)-but-2-enyl, (E)-but-1-enyl, (Z)-but-1-enyl, isopropenyl, 2-methylprop-2-enyl, 1-methylprop-2-enyl, 2-methylprop-1-enyl, (E)-1-methylprop-1-enyl, (Z)-1-methylprop-1-enyl, and the like.

The term “alkynyl” refers to a linear or branched unsaturated aliphatic hydrocarbon group consisting of carbon atoms and hydrogen atoms and having at least one triple bond. The term “C₂-C₁₀ alkynyl” may be understood to represent a linear or branched unsaturated hydrocarbon group comprising one or more triple bonds and having 2, 3, 4, 5, 6, 7, 8, 9, or 10 carbon atoms. Examples of “C₂-C₁₀ alkynyl” include, but are not limited to, ethynyl (—C≡CH), propynyl (—C≡CCH₃, —CH₂C≡CH), but-1-ynyl, but-2-ynyl, and but-3-ynyl. “C₂-C₁₀ alkynyl” may include “C₂-C₃ alkynyl”, and examples of “C₂-C₃ alkynyl” include ethynyl (—C≡CH), prop-i-ynyl (—C≡CCH₃), and prop-2-ynyl (—CH₂C≡CH).

The term “cycloalkyl” refers to a fully saturated carbon ring group that exists in the form of a monocyclic ring, a fused ring, a bridged ring, a spiro ring, or the like. Unless otherwise indicated, the carbon ring is generally a 3-20 membered ring. The term “C₃-C₁₂ cycloalkyl” refers to a cycloalkyl group having 3, 4, 5, 6, 7, 8, 9, 10, 11, or 12 ring carbon atoms. Specific examples of the cycloalkyl include, but are not limited to, cyclopropyl, cyclobutyl, cyclopentyl, cyclohexyl, cycloheptyl, cyclooctyl, cyclononyl, cyclodecyl, norbornyl (bicyclo[2.2.1]heptyl), bicyclo[2.2.2]octyl, adamantyl, spiro[4.5]decyl, and the like. The term “C₃-C₁₂ cycloalkyl” may include the ranges of “C₅-C₁₂ cycloalkyl”, “C₃-C₁₀ cycloalkyl”, “C₃-C₆ cycloalkyl” and the like. The term “C₅-C₁₂ cycloalkyl” refers to a cycloalkyl group having 5, 6, 7, 8, 9, 10, 11, or 12 ring carbon atoms. The term “C₃-C₁₀ cycloalkyl” refers to a cycloalkyl group having 3, 4, 5, 6, 7, 8, 9, or 10 ring carbon atoms. The term “C₃-C₆ cycloalkyl” refers to a cycloalkyl group having 3, 4, 5, or 6 ring carbon atoms.

The term “cycloalkyloxy” may be understood as “cycloalkyl-O—”.

The term “C₃-C₁₀ cycloalkenyl” refers to a non-aromatic carbon ring group that is not fully saturated, has at least one carbon-carbon double bond, exists in the form of a monocyclic ring, a fused ring, a bridged ring, a spiro ring, or the like, and has 3-10 ring carbon atoms. The term “C₅-C₁₀ cycloalkenyl” refers to a cycloalkenyl group having 5, 6, 7, 8, 9, or 10 ring carbon atoms. The term “C₅-C₇ monocyclic alkenyl” refers to a monocyclic alkenyl group having 5, 6, or 7 ring carbon atoms. Specific examples of cycloalkenyl include, but are not limited to, cyclopentenyl, cyclopentadienyl, cyclohexenyl, cyclohexadienyl, cycloheptenyl, cycloheptadienyl, and the like.

The term “heterocyclyl” refers to a fully saturated or partially saturated (non-aromatic heteroaromatic group on the whole) monocyclic, fused cyclic, spiro cyclic, or bridged cyclic group, and ring atoms of the group include 1-5 (e.g., 1-3 or 1-2) heteroatoms or heteroatom groups (i.e., heteroatom-containing atom groups). The “heteroatom or heteroatom group” includes, but is not limited to, a nitrogen atom (N), an oxygen atom (0), a sulfur atom (S), a phosphorus atom (P), a boron atom (B), —S(═O)₂—, —S(═O)—, —P(═O)₂—, —P(═O)—, —NH—, —S(═O)(═NH)—, —C(═O)NH—, —NHC(═O)NH—, and the like. The fused ring, spiro ring, or bridged ring may be bicyclic, tricyclic, tetracyclic, or pentacyclic. The term “12-20 membered tetracyclic heterocyclyl” refers to a tetracyclic heterocyclyl group having 12, 13, 14, 15, 16, 17, 18, 19, or 20 ring atoms, and 1, 2, 3, 4, or 5 ring atoms of the group are independently selected from the heteroatoms and heteroatom groups described above, wherein the tetracyclic ring exists in the form of a fused ring, a spiro ring, a bridged ring, or a combination thereof. The term “10-16 membered tricyclic heterocyclyl” refers to a tricyclic heterocyclyl group having 10, 11, 12, 13, 14, 15, or 16 ring atoms, and 1, 2, 3, 4, or 5 ring atoms of the group are independently selected from the heteroatoms and heteroatom groups described above, wherein the tricyclic ring exists in the form of a fused ring, a spiro ring, a bridged ring, or a combination thereof. The “10-16 membered tricyclic heterocyclyl” may include “12-15 membered tricyclic heterocyclyl”. The term “4-14 membered heterocyclyl” refers to a heterocyclyl group having 4, 5, 6, 7, 8, 9, 10, 11, 12, 13, or 14 ring atoms, and 1, 2, 3, 4 or 5 ring atoms of the group are independently selected from the heteroatoms and heteroatom groups described above. The term “4-10 membered heterocyclyl” refers to a heterocyclyl group having 4, 5, 6, 7, 8, 9, or 10 ring atoms, and 1, 2, 3, 4, or 5 ring atoms of the group are independently selected from the heteroatoms and heteroatom groups described above. The “4-14 membered heterocyclyl” may include “4-10 membered heterocyclyl” and “4-7 membered heterocyclyl”. The “4-10 membered heterocyclyl” may include “4-7 membered heterocyclyl”. The term “4-7 membered heterocyclyl” refers to a heterocyclyl group having 4, 5, 6, or 7 ring atoms, and 1, 2, 3, 4, or 5 ring atoms of the group are independently selected from the heteroatoms or heteroatom groups described above. The term “4-7 membered monoheterocyclyl” refers to a 4-7 membered heterocyclyl group in the form of a monocyclic ring. Specific examples of 4-membered heterocyclyl include, but are not limited to, azetidinyl or oxetanyl; specific examples of 5-membered heterocyclyl include, but are not limited to, tetrahydrofuranyl, dioxolyl, pyrrolidinyl, imidazolidinyl, pyrazolidinyl, pyrrolinyl, 4,5-dihydrooxazolyl, or 2,5-dihydro-1H-pyrrolyl; specific examples of 6-membered heterocyclyl include, but are not limited to, tetrahydropyranyl, piperidinyl, morpholinyl, dithianyl, thiomorpholinyl, piperazinyl, trithianyl, tetrahydropyridyl, or 4H-[1,3,4]thiadiazinyl; specific examples of 7-membered heterocyclyl include, but are not limited to, diazepanyl. The heterocyclyl may also be a bicyclic group, wherein specific examples of 5,5-membered bicyclic groups include, but are not limited to, hexahydrocyclopenta[c]pyrrol-2(1H)-yl; specific examples of 5,6-membered bicyclic groups include, but are not limited to, hexahydropyrrolo[1,2-a]pyrazin-2(1H)-yl, 5,6,7,8-tetrahydro-[1,2,4]triazolo[4,3-a]pyrazinyl, and 5,6,7,8-tetrahydroimidazo[1,5-a]pyrazinyl. Optionally, the heterocyclyl may be a benzo-fused ring group of the 4-7 membered heterocyclyl described above. Specific examples include, but are not limited to, dihydroisoquinolyl and the like. “4-10 membered heterocyclyl” may include the ranges of “5-10 membered heterocyclyl”, “4-7 membered heterocyclyl”, “5-6 membered heterocyclyl”, “6-8 membered heterocyclyl”, “4-10 membered heterocycloalkyl”, “5-10 membered heterocycloalkyl”, “4-7 membered heterocycloalkyl”, “5-6 membered heterocycloalkyl”, “6-8 membered heterocycloalkyl”, and the like, and the “4-7 membered heterocyclyl” may further include the ranges of “4-6 membered heterocyclyl”, “5-6 membered heterocyclyl”, “4-7 membered heterocycloalkyl”, “4-6 membered heterocycloalkyl”, “5-6 membered heterocycloalkyl”, and the like. Although some bicyclic heterocyclyl in the present disclosure comprise, in part, a benzene ring or a heteroaromatic ring, the heterocyclyl is still non-aromatic on the whole.

The term “heterocyclyloxy” may be understood as “heterocyclyl-O—”.

The term “heterocycloalkyl” refers to a fully saturated cyclic group that exists in the form of a monocyclic ring, a fused ring, a bridged ring, a spiro ring, or the like, and ring atoms of the group include 1-5 heteroatoms or heteroatom groups (i.e., heteroatom-containing atom groups). The “heteroatom or heteroatom group” includes, but is not limited to, a nitrogen atom (N), an oxygen atom (O), a sulfur atom (S), a phosphorus atom (P), a boron atom (B), —S(═O)₂—, —S(═O)—, —NH—, —S(═O)(═NH)—, —C(═O)NH—, —NHC(═O)NH—, or the like. The term “4-10 membered heterocycloalkyl” refers to a heterocycloalkyl group having 4, 5, 6, 7, 8, 9, or 10 ring atoms, and 1-5 ring atoms of the groups are independently selected from the heteroatoms and heteroatom groups described above. “4-10 membered heterocycloalkyl” includes “4-7 membered heterocycloalkyl”, wherein specific examples of 4-membered heterocycloalkyl include, but are not limited to, azetidinyl, oxetanyl, and thietanyl; specific examples of 5-membered heterocycloalkyl include, but are not limited to, tetrahydrofuranyl, tetrahydrothienyl, pyrrolidinyl, isoxazolidinyl, oxazolidinyl, isothiazolidinyl, thiazolidinyl, imidazolidinyl, and tetrahydropyrazolyl; specific examples of 6-membered heterocycloalkyl include, but are not limited to, piperidinyl, tetrahydropyranyl, tetrahydrothiopyranyl, morpholinyl, piperazinyl, 1,4-thioxanyl, 1,4-dioxanyl, thiomorpholinyl, 1,3-dithianyl, and 1,4-dithianyl; and specific examples of 7-membered heterocycloalkyl include, but are not limited to, azacycloheptanyl, oxacycloheptanyl, and thiocycloheptanyl.

The term “heterocycloalkyloxy” may be understood as “heterocycloalkyl-O—”.

The term “aryl” refers to an aromatic all-carbon monocyclic or fused polycyclic group with a conjugated π-electron system. Aryl may have 6-20 carbon atoms, 6-14 carbon atoms, or 6-12 carbon atoms. The term “C₆-C₂M aryl” may be understood as an aryl group having 6-20 carbon atoms, e.g., a ring having 6 carbon atoms (“C₆ aryl”), such as phenyl, or a ring having 9 carbon atoms (“C₉ aryl”), such as indanyl or indenyl, or a ring having 10 carbon atoms (“C₁₀ aryl”), such as tetrahydronaphthyl, dihydronaphthyl, or naphthyl, or a ring having 13 carbon atoms (“C₁₃ aryl”), such as fluorenyl, or a ring having 14 carbon atoms (“C₁₄ aryl”), such as anthryl. The term “C₆-C₁₀ aryl” may be understood as an aryl group having 6-10 carbon atoms, e.g., a ring having 6 carbon atoms (“C₆ aryl”), such as phenyl, or a ring having 9 carbon atoms (“C₉ aryl”), such as indanyl or indenyl, or a ring having 10 carbon atoms (“C₁₀ aryl”), such as tetrahydronaphthyl, dihydronaphthyl, or naphthyl.

The term “aryloxy” may be understood as “aryl-O—”.

The term “heteroaryl” refers to an aromatic cyclic group having an aromatic monocyclic or fused polycyclic system, which contains at least one ring atom selected from N, O, and S, with the remaining ring atoms being C, typically has 5, 6, 7, 8, 9, 10, 11, 12, 13, or 14 ring atoms, and comprises 1-8, preferably 1-5, heteroatoms independently selected from N, O, and S. The term “11-14 membered tricyclic heteroaryl” refers to an aromatic cyclic group having an aromatic fused tricyclic system with 11, 12, 13, or 14 ring atoms, wherein at least one ring atom is selected from N, O, and S is present, and the remaining ring atoms are C. The term “5-10 membered heteroaryl” may be understood to include an aromatic monocyclic or bicyclic system that has 5, 6, 7, 8, 9, or 10 ring atoms, e.g., 5, 6, 9, or 10 ring atoms, and comprises 1-5, e.g., 1-3, heteroatoms independently selected from N, O, and S. In particular, the heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, and the like, and benzo derivatives thereof, such as benzofuranyl, benzothienyl, benzothiazolyl, benzoxazolyl, benzoisoxazolyl, benzimidazolyl, benzotriazolyl, indazolyl, indolyl, isoindolyl, and the like; and pyridyl, pyridazinyl, pyrimidinyl, pyrazinyl, triazinyl, and the like, and benzo derivatives thereof, such as quinolyl, quinazolinyl, isoquinolyl, and the like; and azocinyl, indolizinyl, purinyl, and the like, and benzo derivatives thereof, and cinnolinyl, phthalazinyl, quinazolinyl, quinoxalinyl, naphthyridinyl, pteridinyl, carbazolyl, acridinyl, phenazinyl, phenothiazinyl, phenoxazinyl, and the like. The term “5-6 membered heteroaryl” or “5-6 membered monoheteroaryl” refers to an aromatic ring system having 5 or 6 ring atoms and comprising 1-3, e.g., 1-2, heteroatoms independently selected from N, O, and S. The term “5-membered heteroaryl” refers to an aromatic ring system having 5 ring atoms and comprising 1 to 3, e.g., 1 or 2, heteroatoms independently selected from N, O, and S.

The term “heteroaryloxy” may be understood as “heteroaryl-O—”.

The term “halo” or “halogen” refers to fluorine, chlorine, bromine, or iodine.

The term “hydroxy” refers to the —OH group.

The term “cyano” refers to the —CN group.

The term “sulfydryl” refers to the —SH group.

The term “amino” refers to the —NH₂ group.

The term “nitro” refers to the —NO₂ group.

The term “therapeutically effective amount” refers to an amount of the compound of the present disclosure for (i) treating a specific disease, condition, or disorder; (ii) alleviating, ameliorating, or eliminating one or more symptoms of a specific disease, condition, or disorder, or (iii) delaying the onset of the one or more symptoms of the specific disease, condition, or disorder described herein. The amount of the compound of the present disclosure constituting the “therapeutically effective amount” varies dependently on the compound, the disease state and its severity, the administration regimen, and the age of the mammal to be treated, but can be determined routinely by those skilled in the art in accordance with their knowledge and the present disclosure.

The term “prevent”, “preventing”, or “prevention” means administering the compound or formulation described herein to prevent a disease or one or more symptoms associated with the disease, and includes: preventing the occurrence of the disease or disease state in an individual (e.g., a mammal), particularly when such an individual (e.g., a mammal) is predisposed to the disease state but has not yet been diagnosed with it.

The term “individual” includes mammals and non-mammals. Examples of mammals include, but are not limited to, any member of the class Mammalia: humans, non-human primates (e.g., chimpanzees and other apes and monkeys); livestock animals, such as cattle, horses, sheep, goats, and pigs; domestic animals, such as rabbits, dogs, and cats; laboratory animals, including rodents, such as rats, mice, guinea pigs, and the like. Examples of non-human mammals include, but are not limited to, birds, fish, and the like. In one embodiment associated with the methods and compositions provided herein, the mammal is a human. The terms “patient” and “individual” are used interchangeably.

The term “pharmaceutically acceptable” is used herein for those compounds, materials, compositions, and/or dosage forms that are, within the scope of sound medical judgment, suitable for use in contact with the tissues of human beings and animals without excessive toxicity, irritation, allergic response, or other problems or complications, and commensurate with a reasonable benefit/risk ratio.

The term “pharmaceutically acceptable salt” refers to salts of pharmaceutically acceptable acids or bases, including salts formed from the compound and an inorganic or organic acid, and salts formed from the compound and an inorganic or organic base.

The term “pharmaceutical composition” refers to a mixture consisting of one or more of the compounds or the salts thereof of the present disclosure and a pharmaceutically acceptable excipient. The pharmaceutical composition is intended to facilitate the administration of the compound of the present disclosure to an organism.

The term “pharmaceutically acceptable excipient” refers to those that do not have a significant irritating effect on an organism and do not impair the biological activity and properties of the active compound. Suitable excipients are well known to those skilled in the art, such as carbohydrate, wax, water-soluble and/or water-swellable polymers, hydrophilic or hydrophobic materials, gelatin, oil, solvent, water, and the like.

The word “comprise” and variations thereof such as “comprises” or “comprising” may be understood in an open, non-exclusive sense, i.e., “including but not limited to”.

The present disclosure also includes isotopically labeled compounds of the present disclosure which are identical to those documented herein but have one or more atoms replaced by an atom having an atomic mass or mass number different from the atomic mass or mass number usually found in nature. Examples of isotopes that can be incorporated into the compounds of the present disclosure include isotopes of hydrogen, carbon, nitrogen, oxygen, phosphorus, sulfur, fluorine, iodine and chlorine, such as ²H, ³H, ¹¹C, ¹³C, ¹⁴C, ¹³N, ¹⁵N ¹⁵O, ¹⁷O, ¹⁸O, ³¹P, ³²P, ³⁵S, ¹⁸F, ¹²³I, ¹²⁵I, ³⁶Cl, and the like.

Certain isotopically labeled compounds of the present disclosure (e.g., those labeled with ³H and ¹⁴C) can be used to analyze compounds and/or substrate tissue distribution. Tritiated (i.e., ³H) and carbon-14 (i.e., ¹⁴C) isotopes are particularly preferred for their ease of preparation and detectability. Positron emitting isotopes, such as ¹⁵O, ¹³N, ¹¹C, and ¹⁸F, can be used in positron emission tomography (PET) studies to determine substrate occupancy. Isotopically labeled compounds of the present disclosure can generally be prepared by following procedures similar to those disclosed in the schemes and/or examples below while substituting a non-isotopically labeled reagent with an isotopically labeled reagent.

The pharmaceutical composition of the present disclosure can be prepared by combining the compound of the present disclosure with a suitable pharmaceutically acceptable excipient, and can be formulated, for example, into a solid, semisolid, liquid or gaseous formulation such as tablet, pill, capsule, powder, granule, ointment, emulsion, suspension, suppository, injection, inhalant, gel, microsphere, aerosol, and the like.

Typical routes of administration of the compound or the pharmaceutically acceptable salt thereof or the pharmaceutical composition thereof of the present disclosure include, but are not limited to, oral, rectal, local, inhalation, parenteral, sublingual, intravaginal, intranasal, intraocular, intraperitoneal, intramuscular, subcutaneous and intravenous administration.

The pharmaceutical composition of the present disclosure can be manufactured by methods well known in the art, such as conventional methods of mixing, dissolving, granulating, emulsifying, lyophilizing, and the like.

In some embodiments, the pharmaceutical composition is in an oral form. For oral administration, the pharmaceutical composition can be formulated by mixing the active compounds with pharmaceutically acceptable excipients well known in the art. These excipients enable the compounds of the present disclosure to be formulated into tablets, pills, lozenges, dragees, capsules, liquids, gels, slurries, suspensions, and the like, for oral administration to patients.

A solid oral composition can be prepared by conventional mixing, filling or tableting. For example, it can be obtained by the following method: mixing the active compounds with solid excipients, optionally grinding the resulting mixture, adding additional suitable excipients if desired, and processing the mixture into granules to get the core parts of tablets or dragees. Suitable excipients include, but are not limited to, binders, diluents, disintegrants, lubricants, glidants, flavoring agents, and the like.

The pharmaceutical compositions may also be suitable for parenteral administration, such as sterile solutions, suspensions, or lyophilized products in suitable unit dosage forms.

In all of the administration methods for the compound of general formula (I) described herein, the daily dose administered is from 0.01 mg/kg body weight to 200 mg/kg body weight, preferably from 0.05 mg/kg body weight to 50 mg/kg body weight, and more preferably from 0.1 mg/kg body weight to 30 mg/kg body weight, given in a single dose or divided doses.

The compounds of the present disclosure can be prepared by a variety of synthetic methods well known to those skilled in the art, including the specific embodiments listed below, embodiments formed by combinations thereof with other chemical synthetic methods, and equivalents thereof well known to those skilled in the art. The preferred embodiments include, but are not limited to, the examples of the present disclosure.

The chemical reactions of the specific embodiments of the present disclosure are conducted in a suitable solvent that must be suitable for the chemical changes in the present disclosure and the reagents and materials required. In order to obtain the compounds of the present disclosure, it is sometimes necessary for those skilled in the art to modify or select a synthesis procedure or a reaction process based on the existing embodiments.

The following abbreviations are used in the present disclosure:

| Abbreviation | Chemical name | Abbreviation | Chemical name |
|---|---|---|---|
| BPin | 4,4,5,5-tetramethyl-1,3,2- | B₂Pin₂ | 4,4,5,5-tetramethyl-2-(4,4,5,5- |
| | dioxaborane-2-yl | | tetramethyl-1,3,2-dioxaborane-2-yl)- |
| | | | 1,3,2-dioxaborane |
| DCM | Dichloromethane | DMF | N,N-dimethylformamide |
| Pd(dppf)Cl₂ | 1,1′- | HCl | Hydrochloric acid |
| | bis(diphenylphosphino)ferrocene | | |
| | palladium chloride | | |
| DMAP | 4-Dimethylaminopyridine | TFA | Trifluoroacetic acid |
| THF | Tetrahydrofuran | MeCN | Acetonitrile |
| TEA | Triethylamine | NBS | N-Bromosuccinimide |
| M | mol/L | Boc | tert-Butyloxycarbonyl |
| DIEA | N,N-Diisopropylethylamine | MeOH | Methanol |
| DMSO | Dimethyl sulfoxide | TLC | Thin layer chromatography |
| AcOK | Potassium acetate | DIPEA | N,N-Diisopropylethylamine |
| PE | Petroleum ether | EA | Ethyl acetate |
| ACN | Acetonitrile | Pd₂(Ph₃P)₄ | Tetrakis(triphenylphosphine)palladium(0) |
| AIBN | Azobisisobutyronitrile | Pd₂dba₃ | Tris(dibenzylideneacetone)dipalladium(0) |
| Xantphos | 4,5-Bis(diphenylphosphino)-9,9- | Cs₂CO₃ | Cesium carbonate |
| | dimethylxanthene | | |
| XPhos-Pd- | Chloro(2-dicyclohexylphosphino- | XPhos-Pd- | Methanesulfonato(2- |
| G2 | 2′,4′,6′-triisopropyl-1,1′- | G3 | dicyclohexylphosphino-2′,4′,6′- |
| | biphenyl)[2-(2′-amino-1,1′- | | triisopropyl-1,1′-biphenyl)(2′-amino-1,1′- |
| | biphenyl)]palladium(II) | | biphenyl-2-yl)palladium(II) |
| K₃PO₄•3H₂O | Potassium phosphate trihydrate | CaCO₃ | Calcium carbonate |
| THF | Tetrahydrofuran | NaH | Sodium hydride |
| HCHO | Formaldehyde | AcOK | Potassium acetate |
| K₂CO₃ | Potassium carbonate | X-Phos | Dicyclohexyl(2,4,6-triisopropyl-[1,1- |
| | | | biphenyl]-2-yl)phosphine |
| HATU | O-(7-Azabenzotriazol-1-yl)- | TBS | tert-Butyldimethylsilyl |
| | N,N,N′,N′-tetramethyluronium | | |
| | hexafluorophosphate | | |
| TCFH | N,N,N′,N′- | NMI | N-Methylimidazole |
| | Tetramethylchloroformamidinium | | |
| | hexafluorophosphate | | |
| PyBOP | 1H-Benzotriazole-1- | DBU | 1,8-Diazabicyclo[5.4.0]undec-7-ene |
| | yloxytripyrrolidinyl | | |
| | hexafluorophosphate | | |
| RuPhos | 2-Dicyclohexylphosphonium-2′,6′- | | |
| | diisopropoxy-1,1′-biphenyl | | |

### DETAILED DESCRIPTION

The present disclosure is described in detail below by way of examples, which, however, are not intended to disadvantageously limit the scope of the present disclosure in any way. Although the present disclosure has been described in detail herein and specific embodiments thereof have also been disclosed, it will be apparent to those skilled in the art that various changes and modifications can be made to the specific embodiments of the present disclosure without departing from the spirit and scope of the present disclosure. All reagents used in the present disclosure are commercially available and can be used without further purification.

Unless otherwise stated, the ratios expressed for mixed solvents are volume mixing ratios.

Unless otherwise stated, 00 refers to weight percentage wt %.

Compounds are named either manually or by ChemDraw® software, and supplier's catalog names are given for commercially available compounds.

The structures of the compounds are determined by nuclear magnetic resonance (NMR) and/or mass spectrometry (MS). NMR shifts are given in 10⁻⁶ (ppm). The solvents for NMR determination are deuterated dimethyl sulfoxide, deuterated chloroform, deuterated methanol, and the like, and the internal standard is tetramethylsilane (TMS); “IC₅₀” refers to the half maximal inhibitory concentration, which is the concentration at which half of the maximal inhibitory effect is achieved.

The eluent or mobile phase may be a mixed eluent or mobile phase composed of two or more solvents, and the ratio of the mixed eluent or mobile phase is the volume ratio of the solvents. For example, “0-10% methanol/dichloromethane” represents that the volume ratio of methanol to dichloromethane in the mixed eluent or mobile phase is 0:100-10:100.

### Example 1: Synthesis of Compound 1

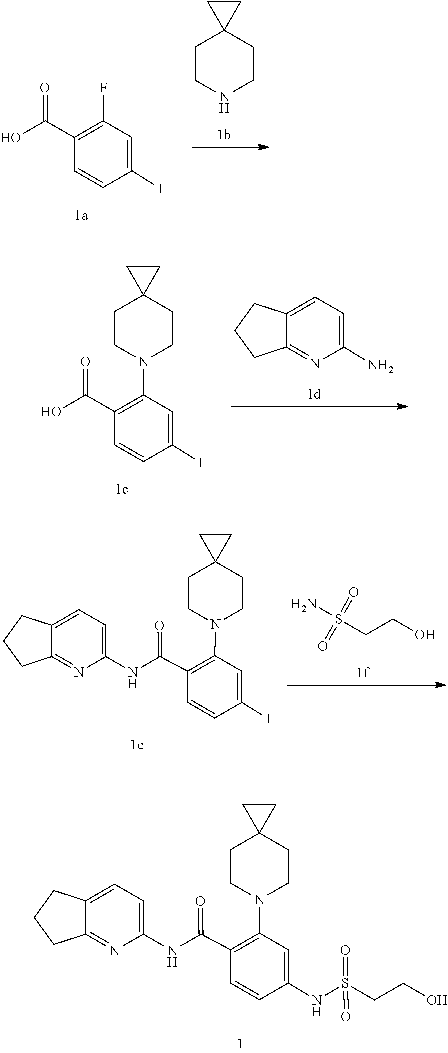

### Step 1: Synthesis of Compound 1c

Compound 1a (2.00 g, 7.52 mmol) and potassium carbonate (3.12 g, 22.5 mmol) were added to DMSO (20 mL), followed by the addition of the hydrochloride of compound 1b (1.22 g, 8.27 mmol) at 25 °C. The reaction mixture was warmed to 140 °C and reacted sufficiently. The reaction mixture was cooled, and water (60 mL) was added to the reaction system. The aqueous phase was washed with petroleum ether (30 mL×2), adjusted to pH=3 with hydrochloric acid (1 N), and filtered, and the filter cake was dried to give compound 1c (1.80 g).

Step 2: synthesis of compound 1e

Compound 1c (30.0 mg, 83.9 μmol), diisopropylethylamine (54.2 mg, 419 μmol), and HATU (47.5 mg, 125 μmol) were added to DMF (1 mL), and after 1 min of sonication, compound 1d (13.5 mg, 100 μmol) was added. The reaction mixture was reacted sufficiently at room temperature. Water (3 mL) was added to the reaction system, and ethyl acetate (2 mL×3) was added for extraction. The combined organic phases were washed with saturated brine, dried over anhydrous magnesium sulfate, and filtered, and the filtrate was concentrated to dryness by rotary evaporation. The residue was purified by flash column chromatography (petroleum ether:ethyl acetate=6:1) to give compound 1e (32 mg).

### Step 3: Synthesis of Compound 1

Compound 1e (20.0 mg, 42.2 μmol), compound if (10.5 mg, 84.5 μmol), potassium phosphate (26.9 mg, 126 μmol), and (1R,2R)—N,N′-dimethyl-1,2-cyclohexanediamine (6.01 mg, 42.25 μmol) were added to DMF (0.5 mL), followed by the addition of cuprous iodide (16.0 mg, 84.5 μmol). The reaction mixture was reacted sufficiently at 95 °C in a nitrogen atmosphere. After the completion of the reaction, the reaction mixture was cooled and directly purified by flash reverse-phase column chromatography (acetonitrile:water, 10-85%) to give compound 1 (8 mg).

MS m/z (ESI): 471.4 [M+H]⁺.

¹H NMR: (400 MHz, DMSO-d₆) δ 12.63 (s, 1H), 8.05 (d, J=8.3 Hz, 1H), 8.00 (d, J=8.7 Hz, 1H), 7.63 (d, J=8.3 Hz, 1H), 7.23 (s, 1H), 7.08 (d, J=8.6 Hz, 1H), 3.75 (t, J=6.4 Hz, 2H), 2.97 (t, J=4.5 Hz, 6H), 2.86 (q, J=7.0 Hz, 4H), 2.12-1.99 (m, 2H), 1.67 (s, 4H), 0.37 (s, 4H).

### Example 2: Synthesis of Compound 2

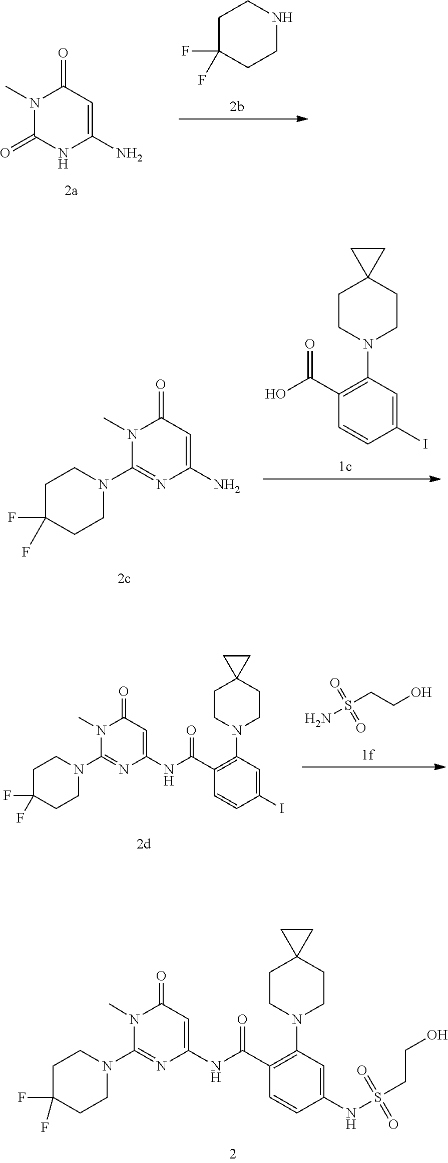

### Step 1: Synthesis of Compound 2c

Compound 2a (1.0 g, 7.1 mmol) and PyBOP (11.3 g, 21.6 mmol) were dissolved in DMF (30 mL), and DBU (11.2 g, 73.8 mmol) and the hydrochloride of compound 2b (1.6 g, 10.1 mmol) were added to the reaction mixture. The reaction mixture was stirred and reacted sufficiently at room temperature. The reaction mixture was dissolved in a mixed solvent of ethyl acetate and water and extracted with ethyl acetate (5×100 mL). The organic phases were combined, dried over anhydrous sodium sulfate, filtered, and purified by flash column chromatography (petroleum ether:ethyl acetate=100:0-0:100) to give compound 2c (820 mg).

¹H NMR (400 MHz, DMSO-d₆) δ 6.22 (s, 2H), 4.86 (s, 1H), 3.24 (s, 3H), 3.21 (t, J=5.7 Hz, 4H), 2.17-2.03 (m, 4H).

### Step 2: Synthesis of Compound 2d

Oxalyl chloride (0.8 mL) and DMF (0.01 mL) were added to a solution of compound 1c (300 mg, 839.9 μmol) in dichloromethane (10 mL) at 0 °C, and the reaction mixture was stirred at room temperature. After the completion of the reaction, the reaction mixture was concentrated to dryness by rotary evaporation. The residue was dissolved in dioxane (10 mL), and the solution was slowly added dropwise to a solution of compound 2c (204.8 mg, 838.6 μmol) and triethylamine (424.3 mg, 4.2 mmol, 584.8 L) in 1,4-dioxane (15 mL) at 0 °C, and the reaction mixture was reacted sufficiently at room temperature. After the completion of the reaction, water (25 mL) was added to quench the reaction, and ethyl acetate (3×30 mL) was added for extraction. The organic phases were combined, concentrated, and purified by flash column chromatography (petroleum ether:ethyl acetate=100:0-50:50) to give compound 2d (180 mg).

MS m/z (ESI): 584.0 [M+H]⁺.

### Step 3: Synthesis of Compound 2

Compound 2 (20 mg) was obtained through synthesis and purification using a method identical to that described in step 3 of Example 1, with compound 2d (50.0 mg, 85.7 μmol) and compound if (21.5 mg, 171.4 μmol) as starting materials.

MS m/z (ESI): 581.1 [M+H]⁺.

¹H NMR (400 MHz, DMSO-d₆) δ 13.13 (s, 1H), 8.03 (d, J=8.7 Hz, 1H), 7.24 (d, J=2.2 Hz, 1H), 7.10 (dd, J=8.7, 2.1 Hz, 1H), 6.83 (s, 1H), 3.75 (t, J=6.5 Hz, 2H), 3.38-3.32 (m, 9H), 2.95 (t, J=5.3 Hz, 4H), 2.15 (m, 4H), 1.71 (m, 4H), 0.38 (s, 4H).

### Example 3: Synthesis of Compound 3

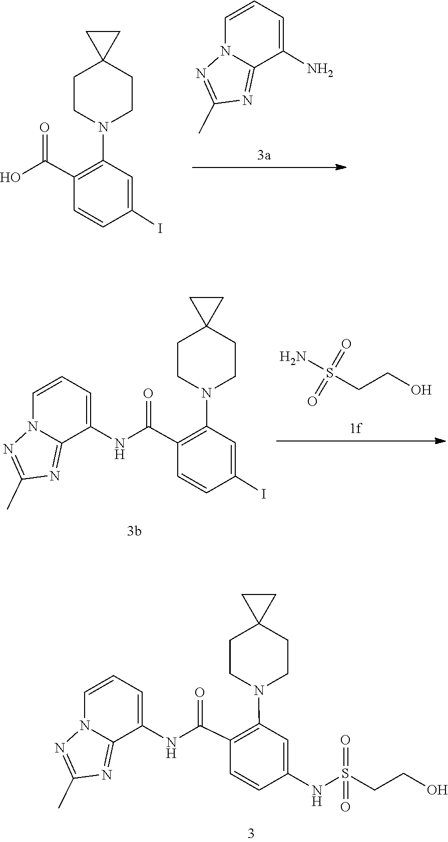 [SMILES: O=C(O)c1ccc(I)cc1N1CCC2(CC1)CC2 ; Cc1nc2c(N)cccn2n1 ; Cc1nc2c(NC(=O)c3ccc(I)cc3N3CCC4(CC3)CC4)cccn2n1 ; NS(=O)(=O)CCO ; Cc1nc2c(NC(=O)c3ccc(NS(=O)(=O)CCO)cc3N3CCC4(CC3)CC4)cccn2n1]

### Step 1: Synthesis of Compound 3b

Compound 3b (33 mg) was obtained through synthesis and purification using a method identical to that described in step 2 of Example 1, with compound 1c (30.0 mg, 83.9 μmol) and compound 3a (14.9 mg, 100 μmol) as starting materials.

### Step 2: Synthesis of Compound 3

Compound 3 (8.1 mg) was obtained through synthesis and purification using a method identical to that described in step 3 of Example 1, with compound 3b (33.0 mg, 67.7 μmol) and compound if (16.9 mg, 135 μmol) as starting materials.

MS m/z (ESI): 485.3 [M+H]⁺.

¹H NMR (400 MHz, DMSO-d₆) δ 12.94 (s, 1H), 8.59 (d, J=7.8 Hz, 1H), 8.54 (d, J=6.7 Hz, 1H), 8.06 (d, J=8.6 Hz, 1H), 7.30 (d, J=2.2 Hz, 1H), 7.15-7.07 (m, 2H), 3.75 (t, J=6.5 Hz, 2H), 3.35 (s, 4H), 2.98 (s, 4H), 2.52 (s, 3H), 1.85 (s, 2H), 0.34 (s, 4H).

### Example 4: Synthesis of Compound 4

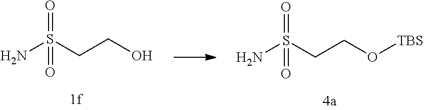

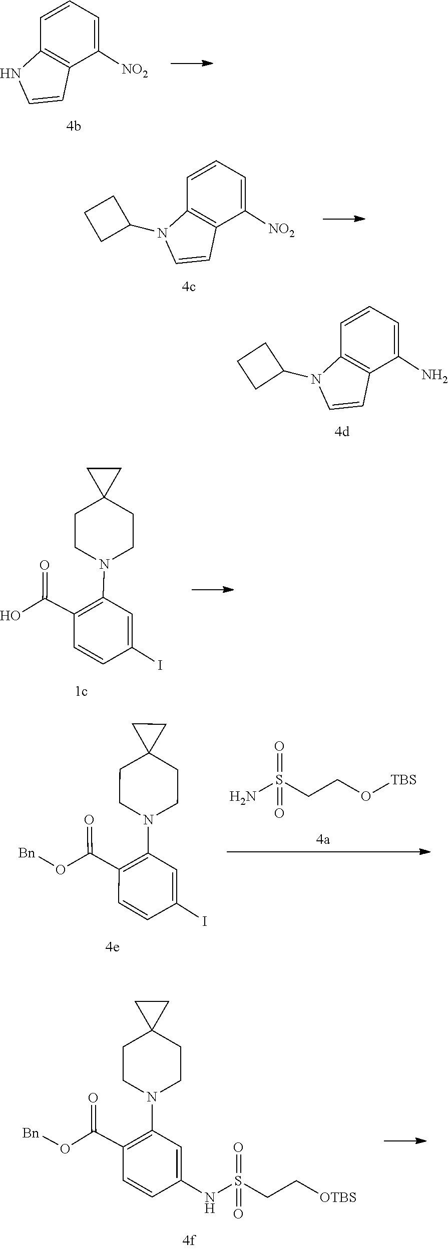

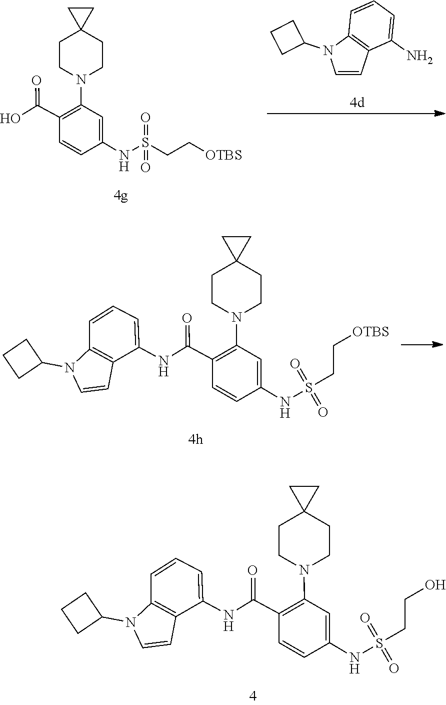 [SMILES: CC(C)(C)[Si](C)(C)OCCS(=O)(=O)Nc1ccc(C(=O)O)c(N2CCC3(CC2)CC3)c1 ; Nc1cccc2c1ccn2C1CCC1 ; CC(C)(C)[Si](C)(C)OCCS(=O)(=O)Nc1ccc(C(=O)Nc2cccc3c2ccn3C2CCC2)c(N2CCC3(CC2)CC3)c1 ; O=C(Nc1cccc2c1ccn2C1CCC1)c1ccc(NS(=O)(=O)CCO)cc1N1CCC2(CC1)CC2]

### Step 1: Synthesis of Compound 4a

Compound 1f (2.00 g, 15.9 mmol) and imidazole (1.31 g, 19.1 mmol) were added to DMF (20 mL), followed by the addition of tert-butyldimethylsilyl chloride (2.89 g, 19.1 mmol) at 0 °C. The reaction mixture was reacted sufficiently at room temperature. A saturated ammonium chloride solution (50 mL) was added, and ethyl acetate (30 mL×3) was added for extraction. The combined organic phases were washed with saturated brine, dried over anhydrous magnesium sulfate, filtered, and slurried with petroleum ether to give compound 4a (3.20 g).

### Step 2: Synthesis of Compound 4c

Compound 4b (200 mg, 1.23 mmol) and cesium carbonate (603 mg, 1.85 mmol) were added to DMF (20 mL), followed by the addition of bromocyclobutane (333 mg, 2.46 mmol). The reaction mixture was reacted sufficiently at 80 °C. After the reaction mixture was cooled, water (30 mL) was added, and ethyl acetate (20 mL×3) was added for extraction. The combined organic phases were washed with saturated brine, dried over anhydrous magnesium sulfate, filtered, and concentrated to dryness by rotary evaporation, and the residue was purified by flash column chromatography (petroleum ether:ethyl acetate=9:1) to give compound 4c (212 mg).

### Step 3: Synthesis of Compound 4d

Compound 4c (100 mg, 0.46 mmol) and ammonium chloride (196 mg, 3.70 mmol) were added to ethanol (2 mL) and water (0.5 mL), followed by the addition of iron powder (103 mg, 1.85 mmol). The reaction mixture was reacted sufficiently at 80 °C. An aqueous sodium hydroxide solution (1 N, 5 mL) was added to the system, and ethyl acetate (3 mL×3) was added for extraction. The combined organic phases were washed with saturated brine, dried over anhydrous magnesium sulfate, and filtered, and the filtrate was concentrated to dryness by rotary evaporation to give compound 4d (59.9 mg).

MS m/z (ESI): 187.2 [M+H]⁺.

### Step 4: Synthesis of Compound 4e

Compound 1c (1.00 g, 2.80 mmol) and potassium carbonate (464 mg, 3.36 mmol) were added to DMF (20 mL), followed by the addition of benzyl bromide (526 mg, 3.08 mmol) at 0 °C. The reaction mixture was reacted sufficiently at room temperature. Water (50 mL) was added to the system, and ethyl acetate (30 mL×3) was added for extraction. The combined organic phases were washed with saturated brine, dried over magnesium sulfate, and filtered, and the filtrate was concentrated to dryness by rotary evaporation. The residue was purified by flash column chromatography (petroleum ether:ethyl acetate=8:1) to give compound 4e (1.0 g).

MS m/z (ESI): 448.2 [M+H]⁺.

### Step 5: Synthesis of Compound 4f

Compound 4f (0.51 g) was obtained through synthesis and purification using a method identical to that described in step 3 of Example 1, with compound 4e (0.50 g, 1.12 mmol) and compound 4a (401 mg, 1.68 mmol) as starting materials.

MS m/z (ESI): 559.3 [M+H]⁺.

### Step 6: Synthesis of Compound 4g

Compound 4f (0.50 g, 1.12 mmol) was added to methanol (6 mL) and ethyl acetate (4 mL), followed by the addition of palladium on carbon (0.20 g, 10%). The reaction mixture was reacted sufficiently at 25 °C in a hydrogen atmosphere. The reaction mixture was filtered, concentrated to dryness by rotary evaporation, and slurried with petroleum ether (5 mL) to give compound 4g (314 mg).

MS m/z (ESI): 469.3 [M+H]⁺.

### Step 7: Synthesis of Compound 4h

Compound 4h (20.0 mg) was obtained through synthesis and purification using a method identical to that described in step 2 of Example 1, with compound 4g (20.0 mg, 42.7 μmol) and compound 4d (11.9 mg, 64.1 μmol) as starting materials.

MS m/z (ESI): 637.5 [M+H]⁺.

### Step 8: Synthesis of Compound 4

Compound 4h (20.0 mg, 31.4 μmol) and pyridine hydrofluoride (9.32 mg, 94.2 μmol) were added to DMF, and the reaction mixture was reacted sufficiently at room temperature. The reaction mixture was directly purified by flash reverse-phase column chromatography (acetonitrile:water, 80%) to give compound 4 (7.2 mg).

MS m/z (ESI): 523.3 [M+H]⁺.

¹H NMR (400 MHz, DMSO-d₆) δ 11.22 (s, 1H), 7.94 (d, J=7.6 Hz, 1H), 7.86 (d, J=8.4 Hz, 1H), 7.60 (d, J=3.3 Hz, 1H), 7.26 (d, J=8.2 Hz, 1H), 7.17 (s, 1H), 7.11 (t, J=8.0 Hz, 1H), 7.01 (d, J=8.5 Hz, 1H), 6.70 (d, J=3.3 Hz, 1H), 5.04-4.90 (m, 1H), 3.76 (t, J=6.6 Hz, 2H), 3.02 (t, J=5.0 Hz, 4H), 2.90-2.74 (m, 1H), 2.49-2.40 (m, 4H), 1.92-1.79 (m, 2H), 1.46 (s, 4H), 1.26-1.08 (m, 1H), 0.32-0.22 (s, 4H).

### Example 5: Synthesis of Compound 5

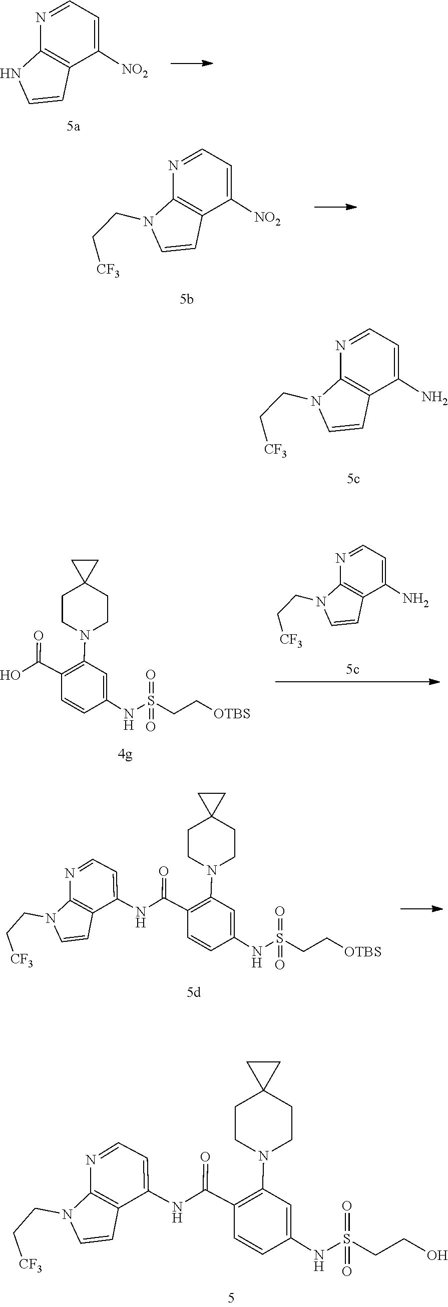

### Step 1: Synthesis of Compound 5b

Compound 5b (0.19 g) was obtained through synthesis and purification using a method identical to that described in step 2 of Example 4, with compound 5a (0.20 g, 1.22 mmol) and 1-iodo-3,3,3-trifluoropropane (1.37 g, 6.13 mmol) as starting materials.

MS m/z (ESI): 260.2 [M+H]⁺.

### Step 2: Synthesis of Compound 5c

Compound 5c (0.12 g) was obtained through synthesis and purification using a method identical to that described in step 3 of Example 4, with compound 5b (0.19 g, 0.73 mmol) as starting material.

MS m/z (ESI): 230.1 [M+H]⁺.

### Step 3: Synthesis of Compound 5d

Compound 4g (20.0 mg, 42.7 μmol), compound 5c (14.7 mg, 64.1 μmol), NMI (35.0 mg, 427 μmol), and TCFH (35.9 mg, 128 μmol) were added to acetonitrile (0.5 mL), and the reaction mixture was reacted sufficiently at 80 °C. Water (3 mL) was added to the system, and ethyl acetate (2 mL×3) was added for extraction. The combined organic phases were washed with saturated brine, dried over anhydrous magnesium sulfate, and filtered, and the filtrate was concentrated to dryness by rotary evaporation. The residue was purified by flash column chromatography (petroleum ether:ethyl acetate=9:1) to give compound 5d (20.0 mg).

MS m/z (ESI): 680.4 [M+H]⁺.

### Step 4: Synthesis of Compound 5

Compound 5 (7.1 mg) was obtained through synthesis and purification using a method identical to that described in step 8 of Example 4, with compound 5d (20.0 mg, 29.4 μmol) as starting material.

MS m/z (ESI): 566.3 [M+H]⁺.

### Example 6: Synthesis of Compound 6

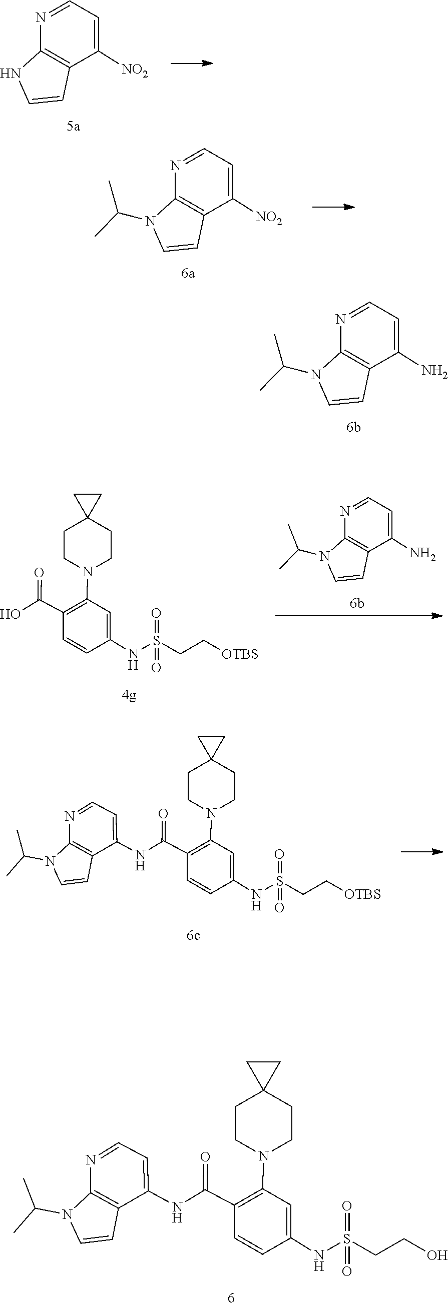

### Step 1: Synthesis of Compound 6a

Compound 6a (0.21 g) was obtained through synthesis and purification using a method identical to that described in step 2 of Example 4, with compound 5a (0.20 g, 1.22 mmol) and 2-iodopropane (0.31 g, 1.83 mmol) as starting materials.

MS m/z (ESI): 206.4 [M+H]⁺.

### Step 2: Synthesis of Compound 6b

Compound 6b (0.13 g) was obtained through synthesis and purification using a method identical to that described in step 3 of Example 4, with compound 6a (0.21 g, 0.99 mmol) as starting material.

MS m/z (ESI): 176.2 [M+H]⁺.

### Step 3: Synthesis of Compound 6c

Compound 6c (20.6 mg) was obtained through synthesis and purification using a method identical to that described in step 3 of Example 5, with compound 4g (20.0 mg, 42.7 μmol) and compound 6b (11.3 mg, 64.1 mmol) as starting materials.

MS m/z (ESI): 626.3 [M+H]⁺.

### Step 4: Synthesis of Compound 6

Compound 6 (8.8 mg) was obtained through synthesis and purification using a method identical to that described in step 8 of Example 4, with compound 6c (20.0 mg, 32.0 μmol) as starting material.

MS m/z (ESI): 512.3 [M+H]⁺.

¹H NMR (400 MHz, Methanol-d₄) δ 8.21 (d, J=5.5 Hz, 1H), 8.09 (d, J=5.4 Hz, 1H), 8.00 (d, J=8.5 Hz, 1H), 7.55 (d, J=4.4 Hz, 1H), 7.30 (s, 1H), 7.11 (d, J=8.5 Hz, 1H), 6.78 (d, J=3.7 Hz, 1H), 5.18-5.08 (m, 1H), 3.97 (t, J=6.3 Hz, 2H), 3.39 (t, J=6.3 Hz, 2H), 3.13 (d, J=5.7 Hz, 4H), 1.59 (s, 4H), 1.56 (d, J=6.8 Hz, 6H), 0.35 (s, 4H).

### Example 7: Synthesis of Compound 7

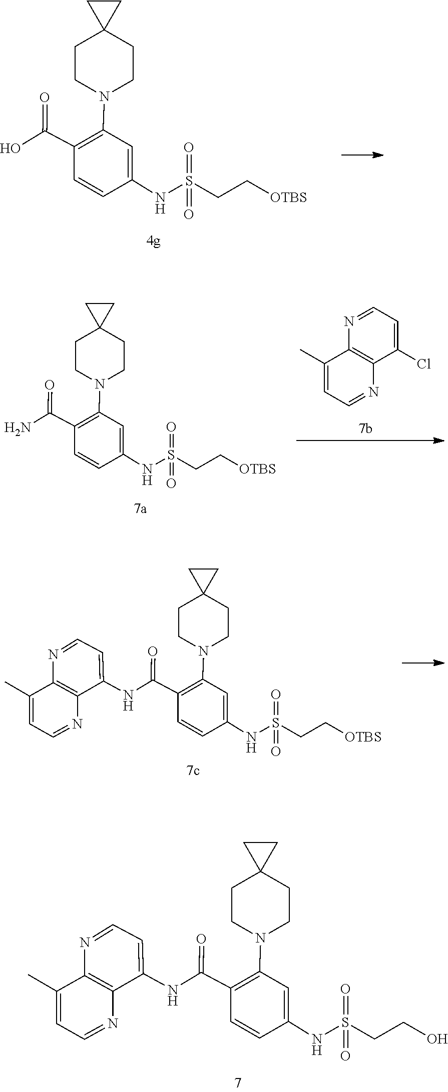 [SMILES: CC(C)(C)[Si](C)(C)OCCS(=O)(=O)Nc1ccc(C(=O)O)c(N2CCC3(CC2)CC3)c1 ; CC(C)(C)[Si](C)(C)OCCS(=O)(=O)Nc1ccc(C(N)=O)c(N2CCC3(CC2)CC3)c1 ; Cc1ccnc2c(Cl)ccnc12 ; Cc1ccnc2c(NC(=O)c3ccc(NS(=O)(=O)CCO[Si](C)(C)C(C)(C)C)cc3N3CCC4(CC3)CC4)ccnc12 ; Cc1ccnc2c(NC(=O)c3ccc(NS(=O)(=O)CCO)cc3N3CCC4(CC3)CC4)ccnc12]

### Step 1: Synthesis of Compound 7a

Compound 4g (30.0 mg, 64.1 μmol), diisopropylethylamine (54.2 mg, 321 μmol), and HATU (36.5 mg, 96.1 μmol) were added to DMF (1 mL), and after 1 min of sonication, ammonium chloride (10.1 mg, 192 mol) was added. The reaction mixture was reacted sufficiently at room temperature. Water (3 mL) was added to the system, and ethyl acetate (2 mL×3) was added for extraction. The combined organic phases were washed with saturated brine, dried over anhydrous magnesium sulfate, and filtered, and the filtrate was concentrated to dryness by rotary evaporation. The residue was purified by flash column chromatography (petroleum ether:ethyl acetate=5:1) to give compound 7a (24.9 mg).

MS m/z (ESI): 468.4 [M+H]⁺.

### Step 2: Synthesis of Compound 7c

Compound 7a (10.0 mg, 21.4 μmol), compound 7b (5.71 mg, 32.1 μmol), and potassium phosphate (9.07 mg, 42.8 μmol) were added to DMF (0.3 mL), followed by the addition of X-Phos (2.03 mg, 4.28 μmol) and Pd₂(dba)₃ (1.96 mg, 2.14 μmol). The reaction mixture was reacted sufficiently at room temperature. Water (2 mL) was added to the system, and ethyl acetate (1 mL×3) was added for extraction. The combined organic phases were washed with saturated brine, dried over anhydrous magnesium sulfate, and filtered, and the filtrate was concentrated to dryness by rotary evaporation. The residue was purified by flash column chromatography (petroleum ether:ethyl acetate=6:1) to give compound 7c (9.07 mg).

MS m/z (ESI): 610.4 [M+H]⁺.

### Step 3: Synthesis of Compound 7

Compound 7 (5.6 mg) was obtained through synthesis and purification using a method identical to that described in step 8 of Example 4, with compound 7c (9.07 mg, 14.9 μmol) as starting material.

MS m/z (ESI): 496.3 [M+H]⁺.

### Example 8: Synthesis of Compound 8

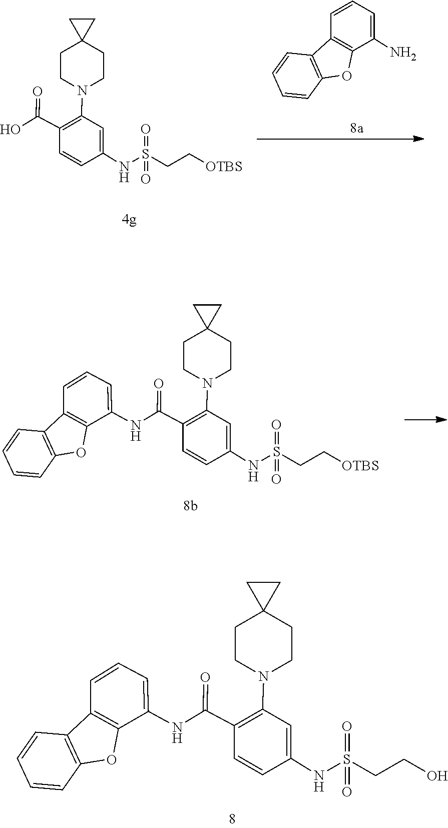 [SMILES: CC(C)(C)[Si](C)(C)OCCS(=O)(=O)Nc1ccc(C(=O)O)c(N2CCC3(CC2)CC3)c1 ; Nc1cccc2c1oc1ccccc12 ; CC(C)(C)[Si](C)(C)OCCS(=O)(=O)Nc1ccc(C(=O)Nc2cccc3c2oc2ccccc23)c(N2CCC3(CC2)CC3)c1 ; O=C(Nc1cccc2c1oc1ccccc12)c1ccc(NS(=O)(=O)CCO)cc1N1CCC2(CC1)CC2]

### Step 1: Synthesis of Compound 8b

Compound 8b (25.0 mg) was obtained through synthesis and purification using a method identical to that described in step 2 of Example 1, with compound 4g (20.0 mg, 42.7 μmol) and compound 8a (11.7 mg, 64.1 μmol) as starting materials.

MS m/z (ESI): 634.1 [M+H]⁺.

### Step 2: Synthesis of Compound 8

Compound 8 (9.6 mg) was obtained through synthesis and purification using a method identical to that described in step 8 of Example 4, with compound 8b (20.0 mg, 31.6 μmol) as starting material.

MS m/z (ESI): 520.1 [M+H]⁺.

### Example 9: Synthesis of Compound 9

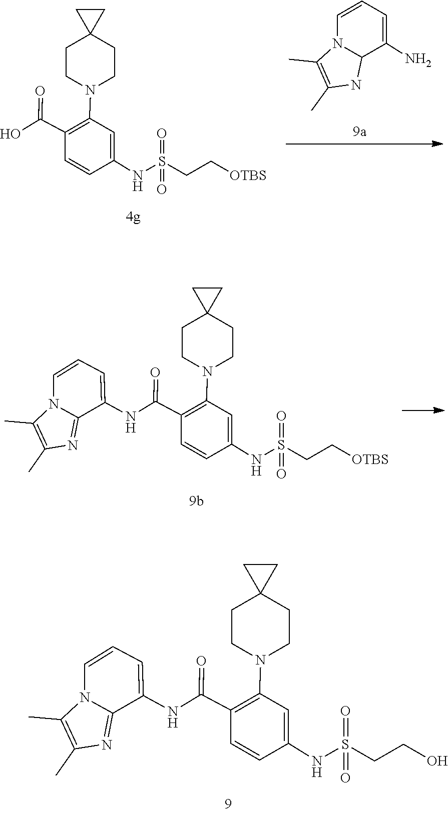 [SMILES: CC(C)(C)[Si](C)(C)OCCS(=O)(=O)Nc1ccc(C(=O)O)c(N2CCC3(CC2)CC3)c1 ; CC1=C(C)N2C=CC=C(N)C2N1 ; Cc1nc2c(NC(=O)c3ccc(NS(=O)(=O)CCO[Si](C)(C)C(C)(C)C)cc3N3CCC4(CC3)CC4)cccn2c1C ; Cc1nc2c(NC(=O)c3ccc(NS(=O)(=O)CCO)cc3N3CCC4(CC3)CC4)cccn2c1C]

### Step 1: Synthesis of Compound 9b

Compound 9b (21.0 mg) was obtained through synthesis and purification using a method identical to that described in step 2 of Example 1, with compound 4g (20.0 mg, 42.7 μmol) and compound 9a (10.3 mg, 64.1 μmol) as starting materials.

MS m/z (ESI): 612.3 [M+H]⁺.

### Step 2: Synthesis of Compound 9

Compound 9 (8.30 mg) was obtained through synthesis and purification using a method identical to that described in step 8 of Example 4, with compound 9b (20.0 mg, 32.7 μmol) as starting material.

MS m/z (ESI): 498.4 [M+H]⁺.

¹H NMR (400 MHz, Methanol-d₄) δ 8.25 (d, J=7.5 Hz, 1H), 8.04 (d, J=8.2 Hz, 1H), 7.87 (d, J=6.8 Hz, 1H), 7.30 (s, 1H), 7.12 (d, J=8.4 Hz, 1H), 6.92 (t, J=7.1 Hz, 1H), 3.97 (t, J=6.3 Hz, 2H), 3.38 (t, J=6.3 Hz, 2H), 3.10 (s, 4H), 2.45 (d, J=16.2 Hz, 6H), 1.73 (s, 2H), 1.29 (s, 2H), 0.32 (s, 4H).

### Example 10: Synthesis of Compound 10

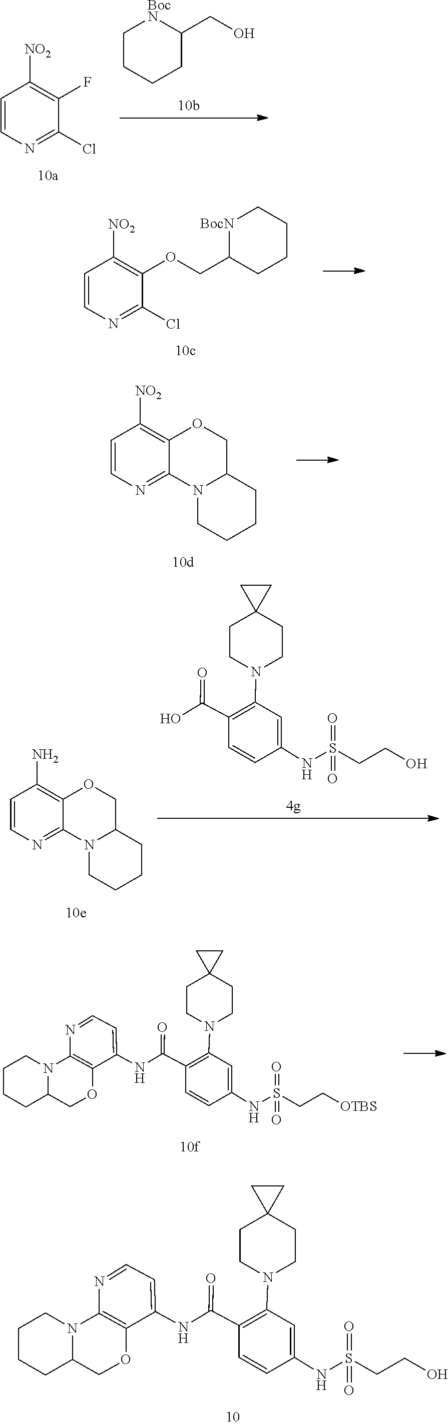

### Step 1: Synthesis of Compound 10c

Compound 10a (1.0 g, 5.66 mmol), compound 10b (1.22 g, 5.66 mmol), and triethylamine (1.14 g, 11.3 mmol) were added to DMF (10 mL), and the reaction mixture was reacted sufficiently at room temperature. Water (20 mL) was added to the system, and ethyl acetate (20 mL×3) was added for extraction. The combined organic phases were washed with saturated brine, dried over anhydrous magnesium sulfate, and filtered, and the filtrate was concentrated to dryness by rotary evaporation. The residue was purified by flash column chromatography (petroleum ether:ethyl acetate=10:1) to give compound 10c (960 mg).

MS m/z (ESI): 372.4 [M+H]⁺.

### Step 2: Synthesis of Compound 10d

Compound 10c (742 mg, 2 mmol) was dissolved in dichloromethane (5 mL), followed by the addition of trifluoroacetic acid (1 mL). The reaction mixture was reacted sufficiently at room temperature. The volatiles were removed under vacuum, and the residue was redissolved in dichloromethane (5 mL), followed by the addition of triethylamine (1 mL). The reaction mixture was reacted sufficiently at room temperature. Water (10 mL) was added to the system, and ethyl acetate (10 mL×3) was added for extraction. The combined organic phases were washed with saturated brine, dried over anhydrous magnesium sulfate, and filtered, and the filtrate was concentrated to dryness by rotary evaporation. The residue was purified by flash column chromatography (petroleum ether:ethyl acetate=8:1) to give compound 10d (60 mg).

MS m/z (ESI): 236.3 [M+H]⁺.

### Step 3: Synthesis of Compound 10e

Compound 10e (18 mg) was obtained through synthesis and purification using a method identical to that described in step 3 of Example 4, with compound 10d (30 mg, 0.13 mmol) as starting material.

MS m/z (ESI): 206.1 [M+H]⁺.

### Step 4: Synthesis of Compound 10f

Compound 10f (24 mg) was obtained through synthesis and purification using a method identical to that described in step 2 of Example 1, with compound 10e (15 mg, 73 μmol) and compound 4g (34 mg, 73 mol) as starting materials.

MS m/z (ESI): 656.5 [M+H]⁺.

### Step 5: Synthesis of Compound 10

Compound 10 (6.3 mg) was obtained through synthesis and purification using a method identical to that described in step 8 of Example 4, with compound 10f (20.0 mg, 30.5 μmol) as starting material.

MS m/z (ESI): 542.3 [M+H]⁺.

¹H NMR (400 MHz, CDCl₃) δ 11.74 (s, 1H), 8.11 (d, J=8.5 Hz, 1H), 7.98 (d, J=5.7 Hz, 1H), 7.78 (d, J=5.8 Hz, 1H), 6.97 (d, J=8.2 Hz, 1H), 4.68 (d, J=13.1 Hz, 1H), 4.29 (d, J=10.0 Hz, 1H), 4.13 (t, J=5.2 Hz, 2H), 4.02 (t, J=9.2 Hz, 1H), 3.41-3.19 (m, 3H), 3.13-2.95 (m, 4H), 2.63 (t, J=12.1 Hz, 1H), 2.02-1.18 (m, 11H), 0.37 (s, 4H).

### Example 11 and Example 12: Synthesis of Compound 11 and Compound 12

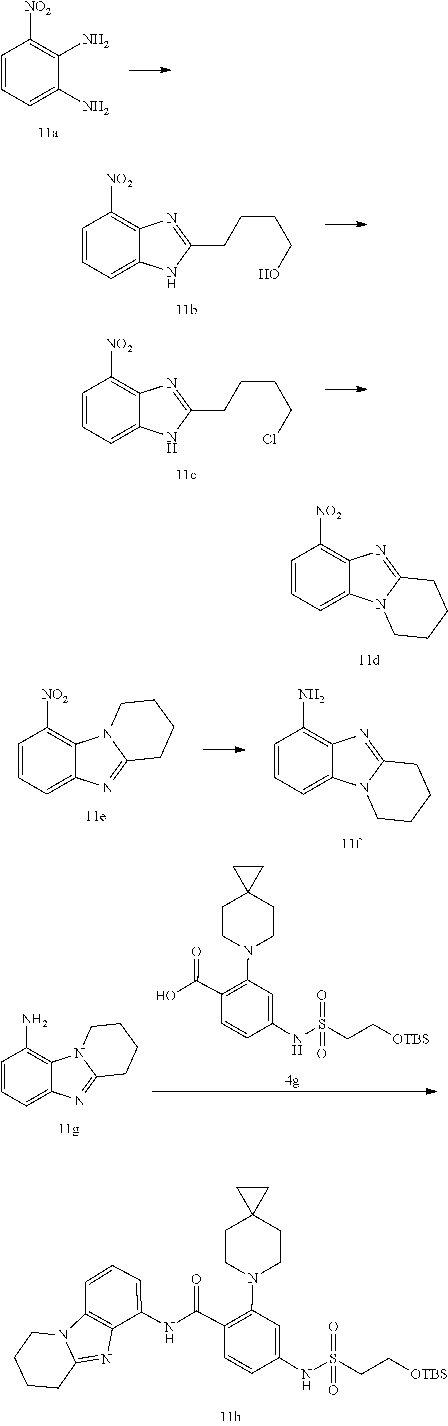

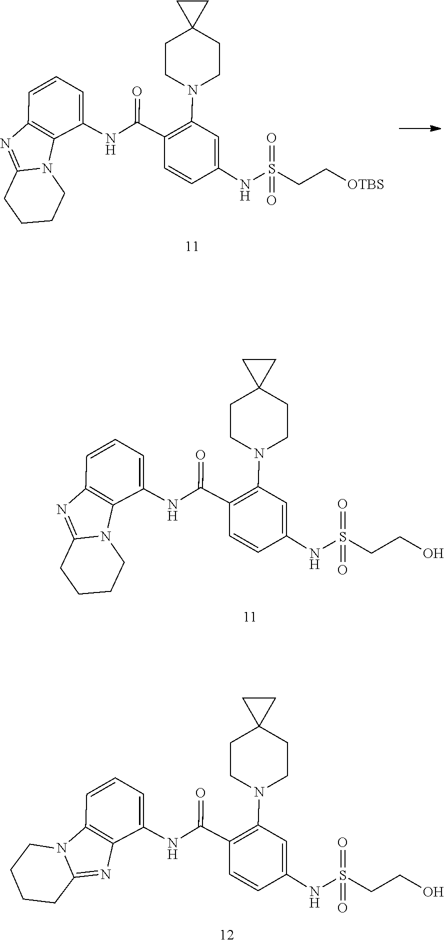 [SMILES: CC(C)(C)[Si](C)(C)OCCS(=O)(=O)Nc1ccc(C(=O)Nc2cccc3nc4n(c23)CCCC4)c(N2CCC3(CC2)CC3)c1 ; O=C(Nc1cccc2nc3n(c12)CCCC3)c1ccc(NS(=O)(=O)CCO)cc1N1CCC2(CC1)CC2 ; O=C(Nc1cccc2c1nc1n2CCCC1)c1ccc(NS(=O)(=O)CCO)cc1N1CCC2(CC1)CC2]

### Step 1: Synthesis of Compound 11b

Compound 11a (1.0 g, 6.54 mmol) and 5-valerolactone (0.65 g, 6.54 mmol) were added to 4 N hydrochloric acid (20 mL), and the reaction mixture was reacted sufficiently under reflux. After the reaction mixture was cooled to room temperature, a 4 N sodium hydroxide solution was added to the system to neutralize to pH 7-8, and ethyl acetate (20 mL×3) was added for extraction. The combined organic phases were washed with saturated brine, dried over anhydrous magnesium sulfate, and filtered, and the filtrate was concentrated to dryness by rotary evaporation. The residue was purified by flash column chromatography (petroleum ether:ethyl acetate=1:1) to give compound 11b (680 mg).

MS m/z (ESI): 236.2 [M+H]⁺.

### Step 2: Synthesis of Compound 11c

Compound 11c (500 mg, 2.13 mmol) was added to thionyl chloride (10 mL), and the reaction mixture was reacted sufficiently under reflux. The volatiles were removed under vacuum, followed by the addition of a saturated sodium bicarbonate solution (10 mL) and the extraction with ethyl acetate (10 mL×3). The combined organic phases were washed with saturated brine, dried over anhydrous magnesium sulfate, and filtered, and the filtrate was concentrated to dryness by rotary evaporation. The residue was purified by flash column chromatography (petroleum ether:ethyl acetate=3:1) to give compound 11c (460 mg).

MS m/z (ESI): 254.2 [M+H]⁺.

Step 3: synthesis of compound lid and compound 11e

Compound 11c (400 mg, 1.57 mmol) was dissolved in anhydrous ethanol (10 mL), followed by the addition of sodium ethoxide (129 mg, 1.89 mmol). The reaction mixture was reacted sufficiently at room temperature. The volatiles were removed under vacuum, followed by the addition of a saturated ammonium chloride solution (10 mL) and the extraction with ethyl acetate (10 mL×3). The combined organic phases were washed with saturated brine, dried over anhydrous magnesium sulfate, and filtered, and the filtrate was concentrated to dryness by rotary evaporation. The residue was purified by flash column chromatography (petroleum ether:ethyl acetate=4:1) to give the mixture (275 mg) of compound lid and compound 11e.

MS m/z (ESI): 218.2 [M+H]⁺.

### Step 4: Synthesis of Compound 11f and Compound 11g

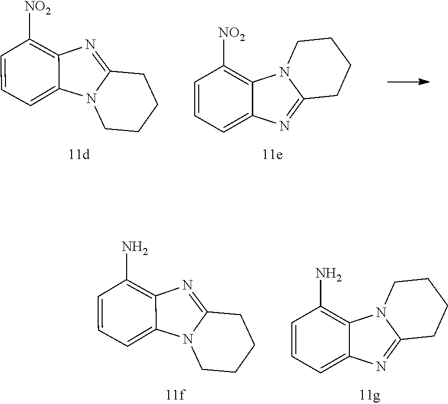

The mixture (118 mg) of compound 11f and compound 11g was obtained through synthesis and purification using a method identical to that described in step 3 of Example 4, with the mixture (217 mg, 1.0 mmol) of compound lid and compound 11e as starting material.

MS m/z (ESI): 188.1 [M+H]⁺.

Step 5: Synthesis of Compound 11 h and compound 11i

The mixture (89 mg) of compound 11 h and compound 11i was obtained through synthesis and purification using a method identical to that described in step 2 of Example 1, with the mixture (40 mg, 214 mol) of compound 11f and compound 11g and compound 4g (100 mg, 214 μmol) as starting materials.

MS m/z (ESI): 638.5 [M+H]⁺.

Step 6: Synthesis of Compound 11 and compound 12

Compound 11 (23 mg) and compound 12 (26 mg) were obtained through synthesis and purification using a method identical to that described in step 8 of Example 4, with the mixture (80.0 mg, 125 μmol) of compound 11 h and compound 11i as starting material.

MS m/z (ESI): 524.3 [M+H]⁺.

### Compound 11

¹H NMR (400 MHz, Methanol-d₄) δ 8.10 (d, J=8.6 Hz, 1H), 7.54 (d, J=8.0 Hz, 1H), 7.37 (s, 1H), 7.27 (t, J=7.8 Hz, 1H), 7.19-7.11 (m, 2H), 4.28 (t, J=5.9 Hz, 2H), 3.96 (t, J=6.1 Hz, 2H), 3.38 (t, J=6.4 Hz, 2H), 3.15 (t, J=5.4 Hz, 4H), 3.06 (t, J=6.2 Hz, 2H), 2.04-1.96 (m, 4H), 1.61 (s, 2H), 0.40 (s, 4H).

### Compound 12

¹H NMR (400 MHz, CDCl₃) δ 12.33 (s, 1H), 8.32 (d, J=8.0 Hz, 1H), 8.04 (d, J=8.4 Hz, 1H), 7.08 (d, J=8.0 Hz, 1H), 6.94 (d, J=8.5 Hz, 1H), 4.13 (s, 2H), 4.04 (s, 2H), 3.27 (s, 2H), 3.15-3.03 (m, 6H), 2.17 (s, 2H), 2.05 (s, 2H), 1.98-1.65 (m, 4H), 0.28 (s, 4H).

### Example 13: Synthesis of Compound 13

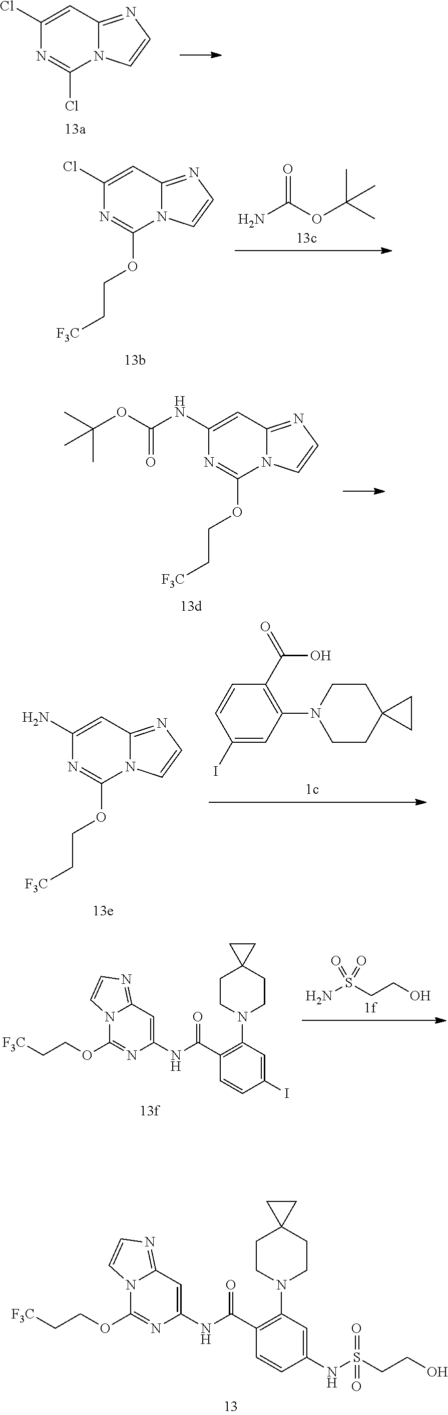

### Step 1: Synthesis of Compound 13b

Trifluoropropanol (328 mg), compound 13a (270 mg), and potassium carbonate (437 mg) were mixed in DMF (20 mL), and the mixture was stirred and reacted sufficiently at 90 °C. After the reaction mixture was cooled to room temperature, water (100 mL) was added, and the mixture was extracted with dichloromethane (3×20 mL). The organic phases were combined and concentrated, and the residue was purified by flash column chromatography (petroleum ether:ethyl acetate=100:0-80:20) to give compound 13b (200 mg).

MS m/z (ESI): 266.0 [M+H]⁺.

### Step 2: Synthesis of Compound 13d

In a nitrogen atmosphere, Pd₂(dba)₃ (65 mg), 4,5-bis(diphenylphosphino)-9,9-dimethylxanthene (Xantphos, 83 mg), and cesium carbonate (0.6 g) were added to a solution of 13b (190 mg) and 13c (170 mg) in dioxane (5 mL), and the reaction mixture was reacted sufficiently under microwaves at 120 °C. After the reaction mixture was cooled to room temperature, water (25 mL) was added, dichloromethane (3×10 mL) was added for extraction. The organic phases were combined and concentrated, and the residue was purified by flash column chromatography (petroleum ether:ethyl acetate=100:0-55:45) to give compound 13d (160 mg).

MS m/z (ESI): 347.1 [M+H]⁺.

### Step 3: Synthesis of Compound 13e

Trifluoroacetic acid (2 mL) was added to a solution of 13d (150 mg) in dichloromethane (5 mL) at 0 °C, and the reaction mixture was reacted sufficiently at room temperature. The volatiles were removed under vacuum, the residue was mixed in dichloromethane (5 mL) and a saturated sodium bicarbonate solution (5 mL), and dichloromethane (3×5 mL) was added for extraction. The organic phases were combined, dried over anhydrous sodium sulfate, and filtered, and the filtrate was concentrated to dryness by rotary evaporation to give 13e (100 mg).

MS m/z (ESI): 247.1 [M+H]⁺.

### Step 4: Synthesis of Compound 13f

Compound 13f (80 mg) was obtained through synthesis and purification using a method identical to that described in step 2 of Example 1, with compound 1c (42 mg) and compound 13e (60 mg) as starting materials.

MS m/z (ESI): 586.1 [M+H]⁺.

### Step 5: Synthesis of Compound 13

Compound 13 (25 mg) was obtained through synthesis and purification using a method identical to that described in step 3 of Example 1, with compound 13f (50 mg) and compound if (54 mg) as starting materials.

MS m/z (ESI): 583.2 [M+H]⁺.

¹H-NMR (600 MHz, DMSO-d₆)δ 13.47 (s, 1H), 10.34 (m, 1H), 8.10 (d, J=8.7 Hz, 1H), 7.94 (s, 1H), 7.66 (s, 1H), 7.52 (s, 1H), 7.29 (d, J=2.2 Hz, 1H), 7.14 (dd, J=8.5, 2.2 Hz, 1H), 4.85 (t, J=5.9 Hz, 2H), 3.77 (t, J=6.5 Hz, 2H), 3.38-3.34 (m, 2H), 3.10-2.96 (m, 6H), 1.76 (m, 4H), 0.41 (s, 4H).

### Example 14: Synthesis of Compound 14

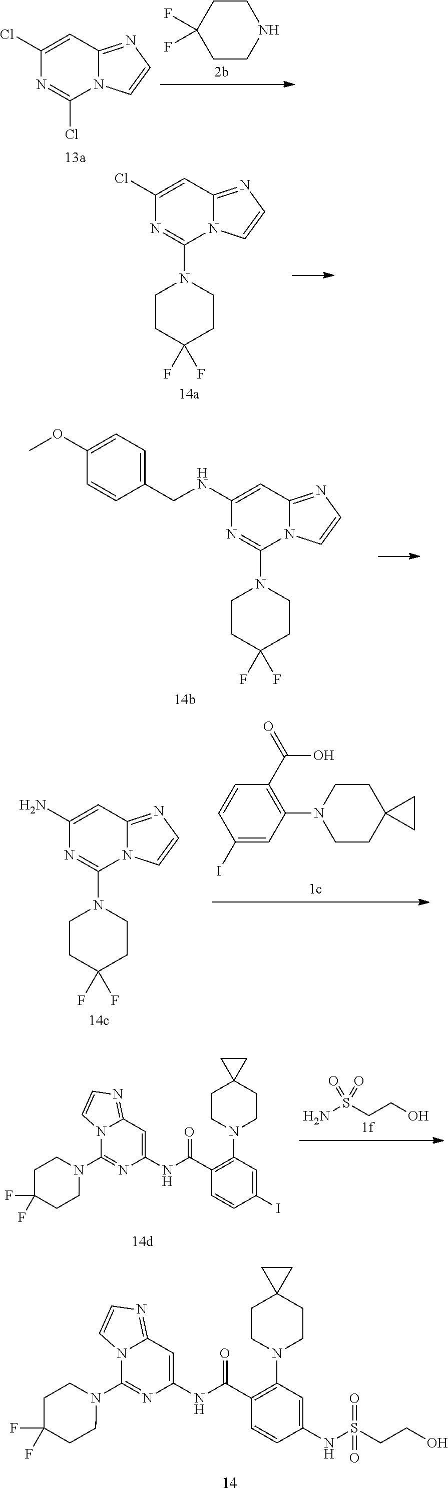

### Step 1: Synthesis of Compound 14a

Compound 14a (650 mg) was obtained through synthesis and purification using a method identical to that described in step 1 of Example 13, with compound 13a (500 mg) and compound 2b (838 mg) as starting materials.

MS m/z (ESI): 273.1 [M+H]⁺.

### Step 2: Synthesis of Compound 14b

Compound 14b (600 mg) was obtained through synthesis and purification using a method identical to that described in step 2 of Example 13, with compound 14a (500 mg) and p-methoxybenzylamine (PMBNH₂, 503 mg) as starting materials.

### Step 3: Synthesis of Compound 14c

Compound 14c (230 mg) was obtained through synthesis and purification using a method identical to that described in step 3 of Example 13, with compound 14b (600 mg) as starting material.

MS m/z (ESI): 254.1 [M+H]⁺.

### Step 4: Synthesis of Compound 14d

Compound 14d (120 mg) was obtained through synthesis and purification using a method identical to that described in step 2 of Example 1, with compound 14c (100 mg) and compound 1c (141 mg) as starting materials.

MS m/z (ESI): 586.1 [M+H]⁺.

### Step 5: Synthesis of Compound 14

Compound 14 (20 mg) was obtained through synthesis and purification using a method identical to that described in step 3 of Example 1, with compound 14d (50 mg) and compound if (50 mg) as starting materials.

MS m/z (ESI): 590.2 [M+H]⁺.

¹H NMR (400 MHz, DMSO-d₆) δ13.30 (s, 1H), 10.47-10.02 (m, 1H), 8.10 (d, J=8.7 Hz, 1H), 7.94 (s, 1H), 7.82 (s, 1H), 7.55 (s, 1H), 7.29 (d, J=2.1 Hz, 1H), 7.14 (dd, J=8.7, 2.1 Hz, 1H), 4.95 (m, 1H), 3.77 (t, J=6.5 Hz, 2H), 3.61 (d, J=6.4 Hz, 4H), 3.33 (m, 2H), 3.00 (s, 4H), 2.29 (m, 4H), 1.76 (m, 4H), 0.41 (s, 4H).

### Example 15: Synthesis of Compound 15

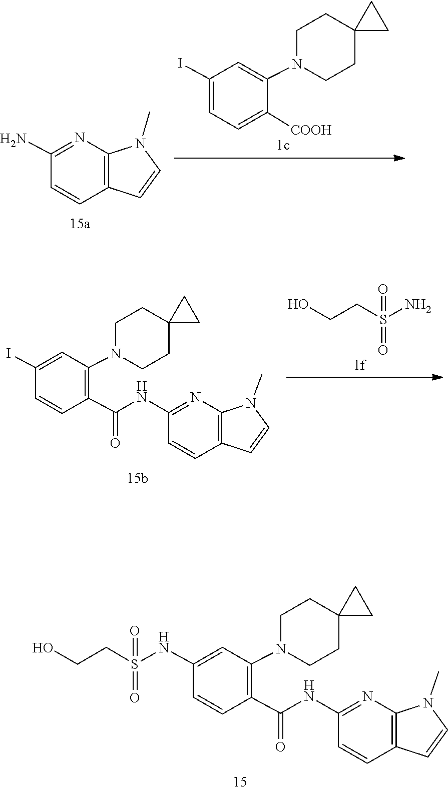 [SMILES: Cn1ccc2ccc(N)nc21 ; O=C(O)c1ccc(I)cc1N1CCC2(CC1)CC2 ; Cn1ccc2ccc(NC(=O)c3ccc(I)cc3N3CCC4(CC3)CC4)nc21 ; NS(=O)(=O)CCO ; Cn1ccc2ccc(NC(=O)c3ccc(NS(=O)(=O)CCO)cc3N3CCC4(CC3)CC4)nc21]

### Step 1: Synthesis of Compound 15b

Compound 15b (60 mg) was obtained through synthesis and purification using a method identical to that described in step 2 of Example 1, with compound 15a (40 mg) and compound 1c (100 mg) as starting materials.

MS m/z (ESI): 487.1 [M+H]⁺.

### Step 2: Synthesis of Compound 15

Compound 15 (23 mg) was obtained through synthesis and purification using a method identical to that described in step 3 of Example 1, with compound 15b (60 mg) and compound if (40 mg) as starting materials.

MS m/z (ESI): 484.1 [M+H]⁺.

¹H NMR (400 MHz, DMSO-d₆) δ13.40 (s, 1H), 10.20 (br, 1H), 8.09 (d, J=8.7 Hz, 2H), 7.99 (d, J=8.4 Hz, 1H), 7.40 (d, J=3.5 Hz, 1H), 7.29 (d, J=2.2 Hz, 1H), 7.14 (dd, J=8.6, 2.1 Hz, 1H), 6.43 (d, J=3.4 Hz, 1H), 4.96 (br, 1H), 3.79 (s, 3H), 3.77 (t, J=6.4 Hz, 2H), 3.36 (t, J=6.6 Hz, 2H), 3.02 (t, J=5.3 Hz, 4H), 1.85 (m, 4H), 0.41 (s, 4H).

### Example 16: Synthesis of Compound 16

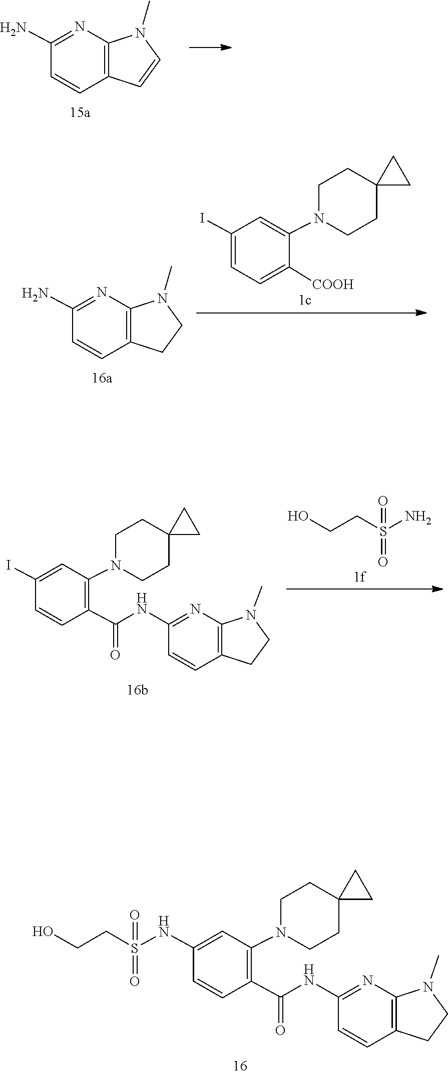 [SMILES: Cn1ccc2ccc(N)nc21 ; CN1CCc2ccc(N)nc21 ; O=C(O)c1ccc(I)cc1N1CCC2(CC1)CC2 ; CN1CCc2ccc(NC(=O)c3ccc(I)cc3N3CCC4(CC3)CC4)nc21 ; NS(=O)(=O)CCO ; CN1CCc2ccc(NC(=O)c3ccc(NS(=O)(=O)CCO)cc3N3CCC4(CC3)CC4)nc21]

### Step 1: Synthesis of Compound 16a

In a H₂ atmosphere, 15a (350 mg) and palladium on carbon (35.0 mg, palladium content 10%) were added to methanol (10 mL), and the reaction mixture was stirred and reacted sufficiently at room temperature. The reaction mixture was filtered and concentrated under vacuum to give 16a (340 mg).

MS m/z (ESI): 150.1 [M+H]⁺

### Step 2: Synthesis of Compound 16b

Compound 16b (450 mg) was obtained through synthesis and purification using a method identical to that described in step 2 of Example 1, with compound 16a (300 mg) and compound 1c (700 mg) as starting materials.

MS m/z (ESI): 489.1 [M+H]⁺.

### Step 3: Synthesis of Compound 16

Compound 16 (110 mg) was obtained through synthesis and purification using a method identical to that described in step 3 of Example 1, with compound 16b (200 mg) and compound if (80 mg) as starting materials.

MS m/z (ESI): 486.2 [M+H]⁺.

¹H NMR (400 MHz, DMSO-d₆) δ12.98 (s, 1H), 10.15 (br, 1H), 8.04 (d, J=8.6 Hz, 1H), 7.38 (d, J=7.6 Hz, 1H), 7.26 (dd, J=7.8, 1.6 Hz, 2H), 7.11 (dd, J=8.6, 2.1 Hz, 1H), 5.02 (br, 1H), 3.76 (t, J=6.5 Hz, 2H), 3.42 (t, J=8.2 Hz, 2H), 3.35 (d, J=6.5 Hz, 2H), 2.97 (t, J=5.3 Hz, 4H), 2.89 (t, J=8.2 Hz, 2H), 2.85 (s, 3H), 1.77 (m, 4H), 0.37 (s, 4H).

### Example 17: Synthesis of Compound 17

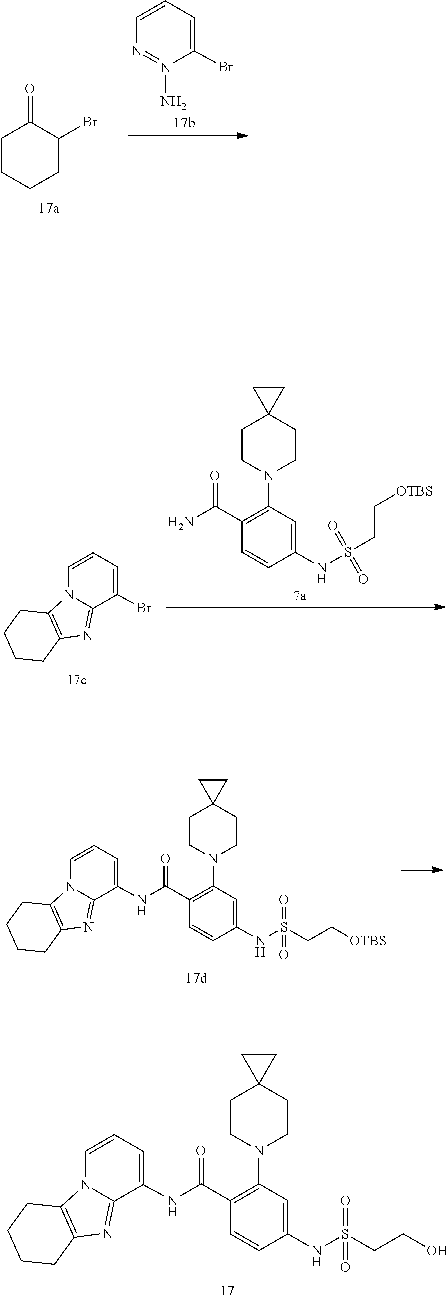

### Step 1: Synthesis of Compound 17c

Compound 17a (100 mg), compound 17b (98 mg), and sodium carbonate (120 mg) were added to ethanol (2 mL), and the reaction mixture was reacted sufficiently at 80 °C. The reaction mixture was cooled to room temperature and then filtered, and the filtrate was purified by reverse-phase column chromatography (acetonitrile:water=10-85%) to give compound 17c (49.7 mg).

MS m/z (ESI): 251.2 [M+H]⁺.

### Step 2: Synthesis of Compound 17d

Compound 17d (6.00 mg) was obtained through synthesis and purification using a method identical to that described in step 2 of Example 7, with compound 17c (12.9 mg) and 7a (20.0 mg) as starting materials.

MS m/z (ESI): 638.3 [M+H]⁺.

### Step 3: Synthesis of Compound 17

Compound 17 (2.96 mg) was obtained through synthesis and purification using a method identical to that described in step 8 of Example 4, with compound 17d (6.00 mg) as starting material.

MS m/z (ESI): 524.5 [M+H]⁺.

¹H NMR (400 MHz, DMSO-d₆) δ 11.92 (s, 1H), 7.72 (d, J=8.5 Hz, 1H), 7.65 (s, 1H), 7.44 (d, J=9.0 Hz, 2H), 7.27 (t, J=8.9 Hz, 1H), 6.91 (s, 1H), 6.78 (d, J=8.2 Hz, 2H), 4.26 (t, J=5.9 Hz, 2H), 3.71 (t, J=6.5 Hz, 2H), 3.35 (t, J=7.1 Hz, 2H), 3.06 (s, 2H), 2.80 (m, 8H), 1.53 (s, 4H), 0.32 (s, 4H).

### Example 18: Synthesis of Compound 18

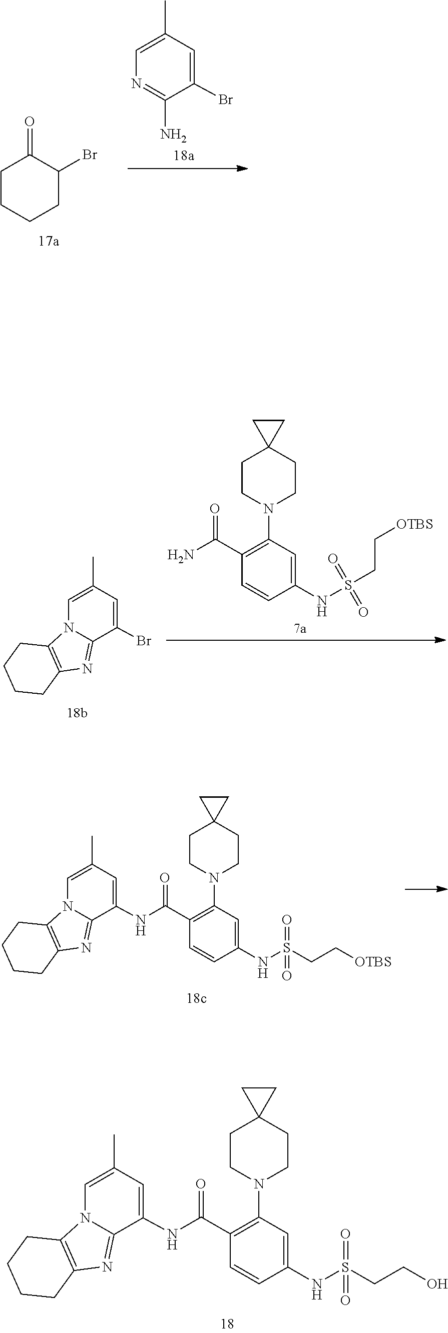

### Step 1: Synthesis of Compound 18b

Compound 18b (42.0 mg) was obtained through synthesis and purification using a method identical to that described in step 1 of Example 17, with compound 17a (100 mg) and compound 18a (106 mg) as starting materials.

MS m/z (ESI): 265.2 [M+H]⁺.

### Step 2: Synthesis of Compound 18c

Compound 18c (8.95 mg) was obtained through synthesis and purification using a method identical to that described in step 2 of Example 7, with compound 18b (12.9 mg) and 7a (20.0 mg) as starting materials.

MS m/z (ESI): 652.2 [M+H]⁺.

### Step 3: Synthesis of Compound 18

Compound 18 (5.30 mg) was obtained through synthesis and purification using a method identical to that described in step 8 of Example 4, with compound 18c (8.95 mg) as starting material.

MS m/z (ESI): 538.3 [M+H]⁺.

### Example 19: Synthesis of Compound 19

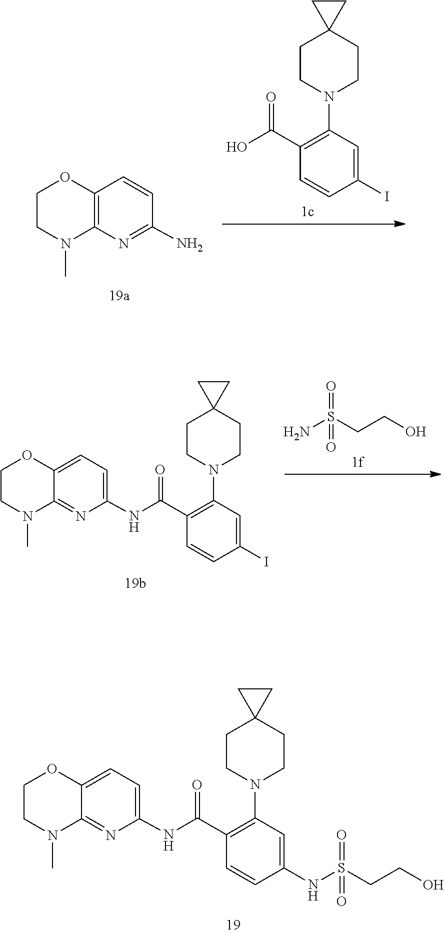 [SMILES: CN1CCOc2ccc(N)nc21 ; O=C(O)c1ccc(I)cc1N1CCC2(CC1)CC2 ; CN1CCOc2ccc(NC(=O)c3ccc(I)cc3N3CCC4(CC3)CC4)nc21 ; NS(=O)(=O)CCO ; CN1CCOc2ccc(NC(=O)c3ccc(NS(=O)(=O)CCO)cc3N3CCC4(CC3)CC4)nc21]

### Step 1: Synthesis of Compound 19b

Compound 19b (220 mg) was obtained through synthesis and purification using a method identical to that described in step 2 of Example 1, with compound 19a (165 mg) and compound 1c (357 mg) as starting materials.

MS m/z (ESI): 505.2 [M+H]⁺.

### Step 2: Synthesis of Compound 19

Compound 19 (15 mg) was obtained through synthesis and purification using a method identical to that described in step 3 of Example 1, with compound 19b (50 mg) and compound if (50 mg) as starting materials.

MS m/z (ESI): 502.2 [M+H]⁺.

¹H NMR (400 MHz, DMSO-d₆) δ 12.90 (s, 1H), 8.04 (d, J=8.5 Hz, 1H), 7.42 (d, J=8.2 Hz, 1H), 7.23 (s, 1H), 7.08 (d, J=8.9 Hz, 1H), 6.96 (d, J=8.2 Hz, 1H), 4.96 (s, 1H), 4.19 (s, 2H), 3.76 (d, J=6.8 Hz, 2H), 3.47-3.41 (m, 4H), 3.06 (s, 3H), 2.96 (s, 4H), 2.16-1.39 (m, 4H), 0.37 (s, 4H).

### Example 20: Synthesis of Compound 20

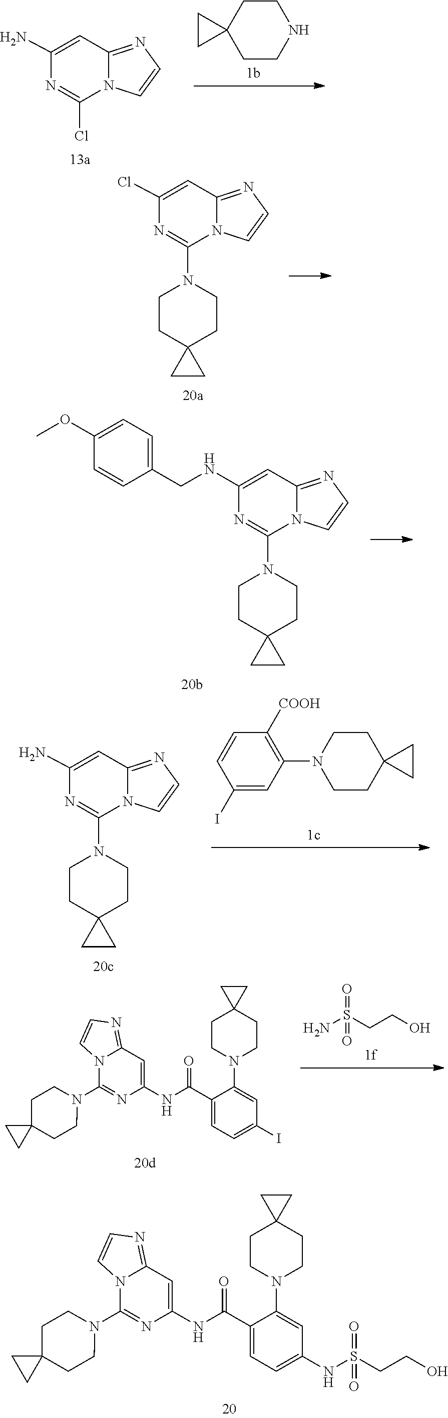

### Step 1: Synthesis of Compound 20a

Compound 20a (270 mg) was obtained through synthesis and purification using a method identical to that described in step 1 of Example 13, with compound 13a (200 mg) and compound 1b (130 mg) as starting materials.

MS m/z (ESI): 263.1 [M+H]⁺.

### Step 2: Synthesis of Compound 20b

Compound 20b (280 mg) was obtained through synthesis and purification using a method identical to that described in step 2 of Example 13, with compound 20a (262 mg) and p-methoxybenzylamine (150 mg) as starting materials.

### Step 3: Synthesis of Compound 20c

Compound 20c (125 mg) was obtained through synthesis and purification using a method identical to that described in step 3 of Example 13, with compound 20b (240 mg) as starting material.

MS m/z (ESI): 244.2 [M+H]⁺.

### Step 4: Synthesis of Compound 20d

Compound 20d (70 mg) was obtained through synthesis and purification using a method identical to that described in step 2 of Example 1, with compound 20c (100 mg) and compound 1c (120 mg) as starting materials.

MS m/z (ESI): 583.3 [M+H]⁺.

### Step 5: Synthesis of Compound 20

Compound 20 (12 mg) was obtained through synthesis and purification using a method identical to that described in step 3 of Example 1, with compound 20d (50 mg) and compound if (50 mg) as starting materials.

MS m/z (ESI): 580.3 [M+H]⁺.

¹H NMR (400 MHz, DMSO-d₆) δ 13.34 (s, 1H), 10.27 (s, 1H), 8.11 (d, J=8.6 Hz, 1H), 7.88 (s, 1H), 7.71 (s, 1H), 7.52 (s, 1H), 7.29 (d, J=2.1 Hz, 1H), 7.15 (dd, J=8.5, 2.0 Hz, 1H), 4.97 (m, 1H), 3.76 (m, 2H), 3.50 (d, J=6.3 Hz, 4H), 3.33 (s, 3H), 2.99 (s, 4H), 1.60 (s, 4H), 0.41 (s, 4H), 0.38 (s, 4H).

### Example 21: Synthesis of Compound 21

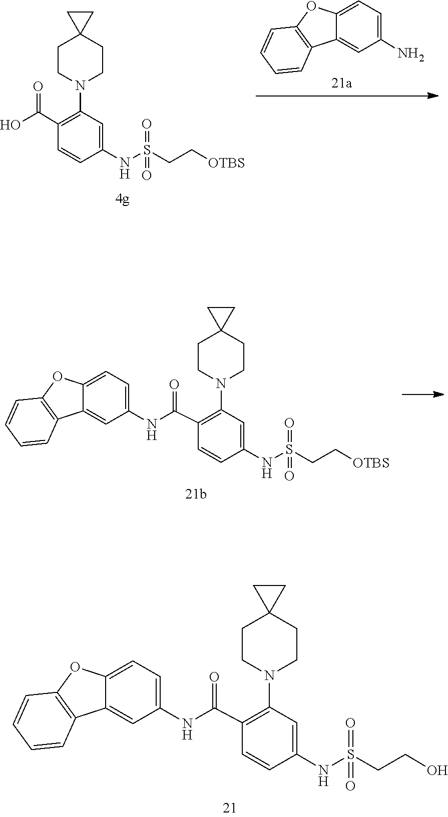 [SMILES: CC(C)(C)[Si](C)(C)OCCS(=O)(=O)Nc1ccc(C(=O)O)c(N2CCC3(CC2)CC3)c1 ; Nc1ccc2oc3ccccc3c2c1 ; CC(C)(C)[Si](C)(C)OCCS(=O)(=O)Nc1ccc(C(=O)Nc2ccc3oc4ccccc4c3c2)c(N2CCC3(CC2)CC3)c1 ; O=C(Nc1ccc2oc3ccccc3c2c1)c1ccc(NS(=O)(=O)CCO)cc1N1CCC2(CC1)CC2]

### Step 1: Synthesis of Compound 21b

Compound 21b (43 mg) was obtained through synthesis and purification using a method identical to that described in step 2 of Example 1, with compound 4g (47 mg) and compound 21a (20 mg) as starting materials.

MS m/z (ESI): 634.2 [M+H]⁺.

### Step 2: Synthesis of Compound 21

Compound 21 (28 mg) was obtained through synthesis and purification using a method identical to that described in step 8 of Example 4, with compound 21b (40.0 mg) as starting material.

MS m/z (ESI): 520.2 [M+H]⁺.

¹H NMR (400 MHz, DMSO-d₆) δ 11.60 (s, 1H), 10.11 (s, 1H), 8.74 (d, J=2.1 Hz, 1H), 8.13 (d, J=7.6 Hz, 1H), 7.85 (d, J=8.4 Hz, 1H), 7.73 (m, 2H), 7.65 (dd, J=8.8, 2.2 Hz, 1H), 7.57-7.51 (m, 1H), 7.42 (m, 1H), 7.18 (d, J=2.0 Hz, 1H), 7.05 (dd, J=8.5, 1.9 Hz, 1H), 4.99 (m, 1H), 3.77 (m, 2H), 3.35 (s, 2H), 3.02 (m, 4H), 1.54 (s, 4H), 0.34 (s, 4H).

### Example 22: Synthesis of Compound 22

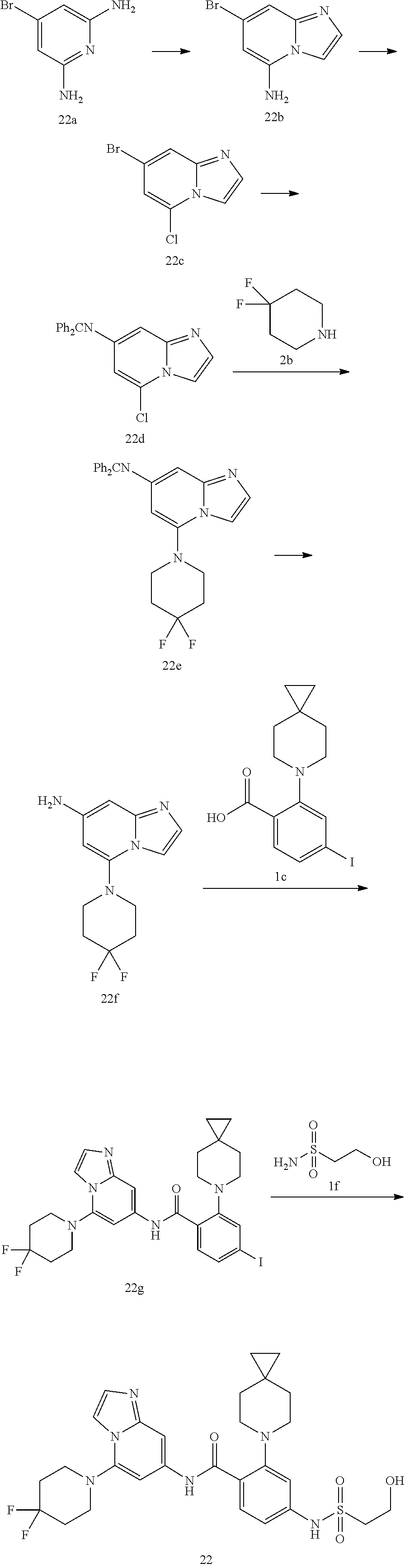

### Step 1: Synthesis of Compound 22b

Compound 22a (4.0 g, 21.3 mmol) and chloroacetaldehyde (2.5 g, 31.9 mmol) were added to a mixed solution of ethanol (84 mL) and water (6 mL), and the mixture was reacted sufficiently under reflux. The mixture was concentrated under reduced pressure to remove most of the organic solvent, followed by the addition of a saturated sodium bicarbonate solution (40 mL). The aqueous phase was extracted with ethyl acetate (40 mL×2). The organic phase was dried over anhydrous sodium sulfate, and the solvent was removed under reduced pressure to give compound 22b (3 g).

MS m/z (ESI): 212.0 [M+H]⁺.

### Step 2: Synthesis of Compound 22c

tert-Butyl nitrite (2.9 g, 28.3 mmol) and copper chloride (3.8 g, 28.3 mmol) were added to acetonitrile (60 mL) at 0 °C, followed by the addition of compound 22b (3.0 g, 14.2 mmol) in batches. The reaction mixture was warmed to room temperature, stirred, and reacted sufficiently. A saturated sodium bicarbonate solution (60 mL) was added to the reaction mixture, and the aqueous phase was extracted with ethyl acetate (60 mL×3). The organic phases were combined and concentrated to give compound 22c (1.5 g).

MS m/z (ESI): 231.0 [M+H]⁺.

### Step 3: Synthesis of Compound 22d

In a nitrogen atmosphere, compound 22c (1.5 g, 6.5 mmol), benzophenone imine (1.8 g, 9.7 mmol), Pd₂dba₃ (593.4 mg, 648.0 μmol), RuPhos (604.8 mg, 1.3 mmol), and cesium carbonate (6.3 g, 19.4 mmol) were added to DMF (10 mL), and the mixture was reacted sufficiently at 110 °C. After the reaction mixture was cooled, water (50 mL) was added, and the aqueous phase was extracted with ethyl acetate (50 mL×2). The organic phases were combined, dried over anhydrous sodium sulfate, filtered, and concentrated under reduced pressure, and the residue was purified by flash column chromatography (petroleum ether:ethyl acetate=100:0-75:25) to give compound 22d (0.3 g).

### Step 4: Synthesis of Compound 22e

In a nitrogen atmosphere, compound 22d (150 mg, 452 μmol) and compound 2b (hydrochloride, 85.5 mg, 542 μmol), Pd₂dba₃ (41.4 mg, 45 μmol), RuPhos (42.2 mg, 90 μmol), and cesium carbonate (442 mg, 1.4 mmol) were added to DMF (3 mL), and the mixture was reacted sufficiently at 110 °C. After the reaction mixture was cooled, water (15 mL) was added, and the aqueous phase was extracted with ethyl acetate (15 mL×2). The organic phases were combined, dried over anhydrous sodium sulfate, filtered, and concentrated under reduced pressure, and the residue was purified by flash column chromatography (petroleum ether:ethyl acetate=100:0-75:25) to give compound 22e (150 mg).

### Step 5: Synthesis of Compound 22f

A solution of hydrogen chloride in dioxane (4 N, 3.5 mL) was added dropwise to a solution of compound 22e (100 mg, 240 μmol) in dioxane (7 mL), and the mixture was reacted sufficiently at room temperature. Water (10 mL) was added to the reaction mixture, and the mixture was washed with ethyl acetate (10 mL×2). The organic phase was discarded, and the aqueous phase was adjusted to pH 8 with a saturated sodium bicarbonate solution and then extracted with ethyl acetate (10 mL×2). The organic phases were combined, dried over anhydrous sodium sulfate, filtered, and concentrated under reduced pressure to give compound 22f (55 mg).

MS m/z (ESI): 253.1 [M+H]⁺.

### Step 6: Synthesis of Compound 22g

Compound 22g (16 mg) was obtained through synthesis and purification using a method identical to that described in step 2 of Example 1, with compound 1c (86 mg, 0.24 mmol) and compound 22f (55 mg, 0.22 mmol) as starting materials.

MS m/z (ESI): 592.1 [M+H]⁺.

### Step 7: Synthesis of Compound 22

Compound 22 (3 mg) was obtained through synthesis and purification using a method identical to that described in step 3 of Example 1, with compound 22f (16 mg, 27 μmol) and compound if (6.4 mg, 54 mol) as starting materials.

MS m/z (ESI): 589.2 [M+H]⁺.

¹H NMR (400 MHz, DMSO-d₆) δ 11.80 (s, 1H), 7.86 (s, 1H), 7.79 (d, J=8.5 Hz, 1H), 7.73 (s, 1H), 7.53 (s, 1H), 7.13 (s, 1H), 7.01 (d, J=8.3 Hz, 1H), 6.81 (s, 1H), 3.76 (t, J=6.5 Hz, 3H), 3.58-3.51 (m, 2H), 3.24-3.21 (m, 4H), 2.99-2.95 (m, 4H), 2.31-2.23 (m, 4H), 2.04-1.93 (m, 4H), 0.34 (s, 4H).

### Example 23: Synthesis of Compound 23

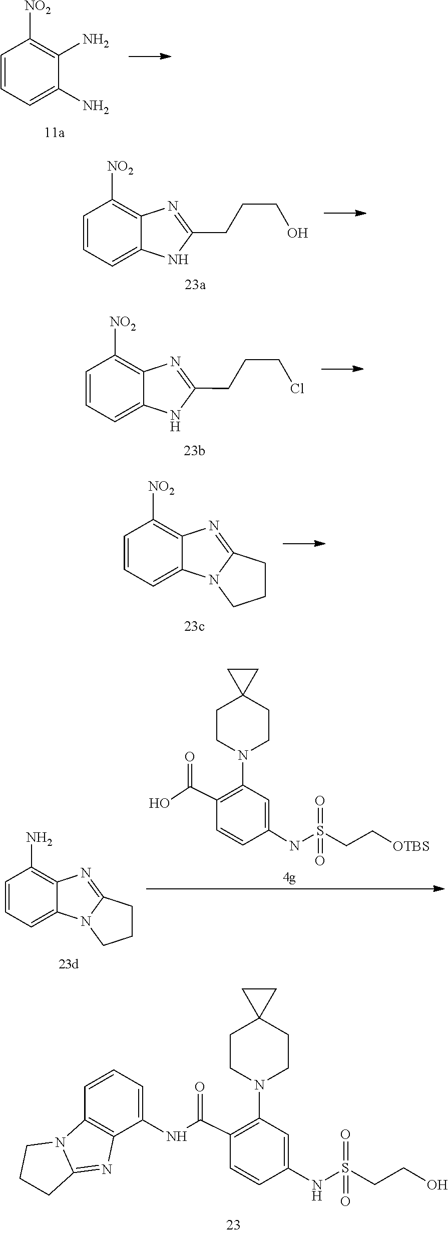

### Step 1: Synthesis of Compound 23a

Compound 23a (570 mg) was obtained through synthesis and purification using a method identical to that described in step 1 of Example 12, with compound 11a (800 mg, 5.23 mmol) and 4-butyrolactone (860 mg, 10.0 mmol) as starting materials.

MS m/z (ESI): 222.0 [M+H]⁺.

### Step 2: Synthesis of Compound 23b

Compound 23b (450 mg) was obtained through synthesis and purification using a method identical to that described in step 2 of Example 12, with compound 23a (500 mg, 2.25 mmol) as starting material.

MS m/z (ESI): 240.0 [M+H]⁺.

### Step 3: Synthesis of Compound 23c

Compound 23c (35 mg) was obtained through synthesis and purification using a method identical to that described in step 3 of Example 12, with compound 23b (400 mg, 1.67 mmol) as starting material.

MS m/z (ESI): 204.0 [M+H]⁺.

### Step 4: Synthesis of Compound 23c

Compound 23d (27 mg) was obtained through synthesis and purification using a method identical to that described in step 3 of Example 4, with compound 23c (35 mg, 0.17 mmol) as starting material.

MS m/z (ESI): 174.1 [M+H]⁺.

### Step 5: Synthesis of Compound 23

Compound 4g (46.8 mg, 100 μmol), diisopropylethylamine (38.7 mg, 300 μmol), and HATU (45.6 mg, 120 μmol) were added to DMF (0.5 mL), and after 1 min of stirring, compound 23d (17.3 mg, 100 μmol) was added. The reaction mixture was reacted sufficiently at room temperature. Pyridine hydrofluoride (14.9 mg, 150 μmol) was added, and the reaction mixture was reacted sufficiently at room temperature. The reaction mixture was directly purified by flash reverse-phase column chromatography (acetonitrile:water=10:90-90:10) to give compound 23 (10.7 mg).

MS m/z (ESI): 510.2 [M+H]⁺.

¹H NMR (400 MHz, DMSO-d₆) δ 12.70 (s, 1H), 8.18 (dd, J=12.9, 2.4 Hz, 1H), 8.05 (d, J=8.6 Hz, 1H), 7.29 (d, J=2.1 Hz, 1H), 7.12-7.06 (m, 2H), 4.09 (t, J=5.9 Hz, 2H), 3.76 (t, J=6.5 Hz, 2H), 3.37 (t, J=6.4 Hz, 2H), 3.05-2.97 (m, 4H), 2.07 (m, 2H), 1.98 (m, 2H), 1.97-1.55 (br, 4H), 0.32 (s, 4H).

### Example 24: Synthesis of Compound 24

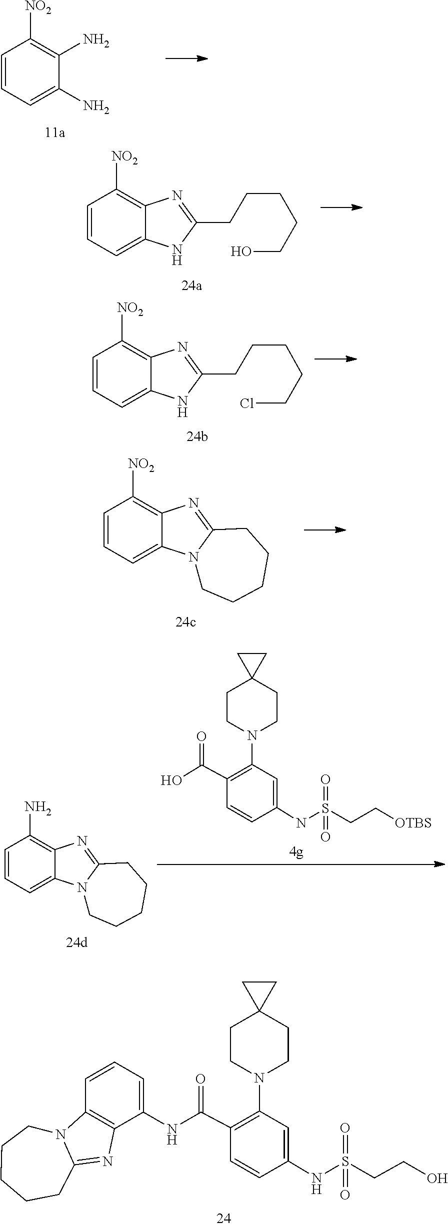

### Step 1: Synthesis of Compound 24a

Compound 24a (745 mg) was obtained through synthesis and purification using a method identical to that described in step 1 of Example 12, with compound 11a (1.0 g, 6.5 mmol) and 6-caprolactone (1.48 g, 13.0 mmol) as starting materials.

MS m/z (ESI): 250.0 [M+H]⁺.

### Step 2: Synthesis of Compound 24b

Compound 24b (425 mg) was obtained through synthesis and purification using a method identical to that described in step 2 of Example 12, with compound 24a (498 mg, 2.0 mmol) as starting material.

MS m/z (ESI): 268.0 [M+H]⁺.

### Step 3: Synthesis of Compound 24c

Compound 24c (136 mg) was obtained through synthesis and purification using a method identical to that described in step 3 of Example 12, with compound 24b (400 mg, 1.5 mmol) as starting material.

MS m/z (ESI): 232.1 [M+H]⁺.

### Step 4: Synthesis of Compound 24d

Compound 24d (71 mg) was obtained through synthesis and purification using a method identical to that described in step 3 of Example 4, with compound 24c (120 mg, 0.52 mmol) as starting material.

MS m/z (ESI): 202.1 [M+H]⁺.

### Step 5: Synthesis of Compound 24

Compound 24 (27.3 mg) was obtained through synthesis and purification using a method identical to that described in step 5 of Example 23, with compound 24d (40 mg, 0.2 mmol) and compound 4g (94 mg, 0.2 mmol) as starting materials.

MS m/z (ESI): 538.2 [M+H]⁺.

¹H NMR (400 MHz, DMSO-d₆) δ 12.26 (s, 1H), 8.00 (d, J=8.6 Hz, 2H), 8.05 (d, J=8.6 Hz, 2H), 7.26 (br, 3H), 7.09 (dd, J=8.6, 2.1 Hz, 1H), 4.39 (br, 2H), 3.77 (t, J=6.5 Hz, 2H), 3.36 (t, J=6.5 Hz, 2H), 3.01 (br, 6H), 1.91 (m, 2H), 1.81 (m, 2H), 1.79-1.45 (br, 6H), 0.31 (s, 4H).

### Example 25: Synthesis of Compound 25

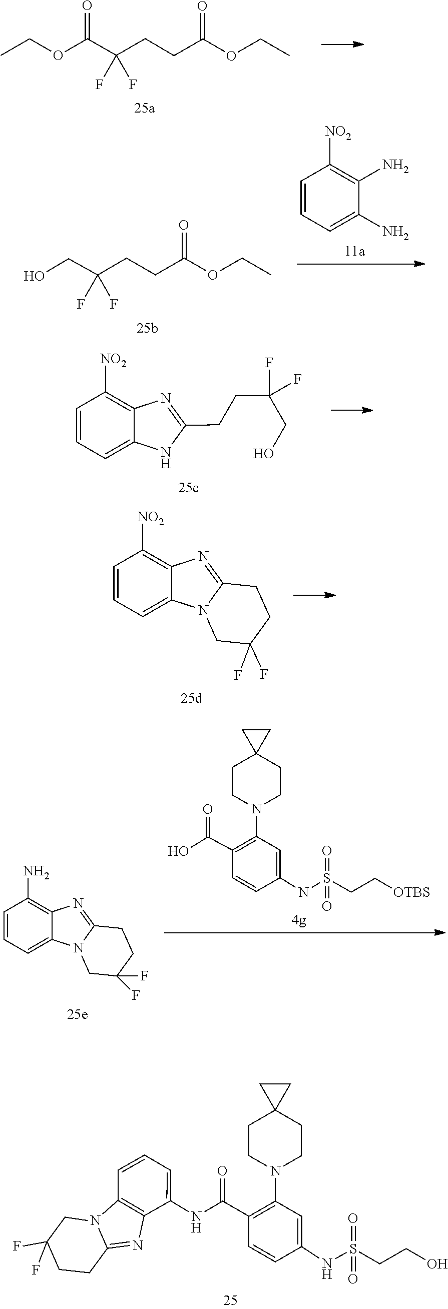

### Step 1: Synthesis of Compound 25b

Compound 25a (1.0 g, 4.5 mmol) was added to a mixed solution of THF (10 mL) and ethanol (10 mL), followed by the addition of sodium borohydride (0.46 g, 13.5 mmol) at 0 °C. The mixture was stirred and reacted sufficiently at room temperature. A saturated ammonium chloride solution (20 mL) was added to quench the reaction, and ethyl acetate (20 mL×3) was added for extraction. The combined organic phases were dried over anhydrous sodium sulfate and filtered, and the solvent was removed from the filtrate under reduced pressure to give compound 25b (0.58 g).

### Step 2: Synthesis of Compound 25c

Compound 25c (320 mg) was obtained through synthesis and purification using a method identical to that described in step 1 of Example 12, with compound 11a (0.41 g, 2.7 mmol) and compound 25b (0.50 g, 2.7 mmol) as starting materials.

MS m/z (ESI): 272.0 [M+H]⁺.

### Step 3: Synthesis of Compound 25d

Compound 25c (0.30 g, 1.1 mmol) and triphenylphosphine (0.39 g, 1.5 mmol) were added to anhydrous THE (5 mL), followed by the dropwise addition of diethyl azodicarboxylate (0.23 g, 1.3 mmol) at 0 °C. The mixture was stirred and reacted sufficiently at room temperature. The solvent was removed under reduced pressure, and the residue was purified by flash column chromatography (petroleum ether:ethyl acetate=1:1) to give compound 25d (183 mg).

MS m/z (ESI): 254.0 [M+H]⁺.

### Step 4: Synthesis of Compound 25e

Compound 25e (90 mg) was obtained through synthesis and purification using a method identical to that described in step 3 of Example 4, with compound 25d (165 mg, 0.65 mmol) as starting material.

MS m/z (ESI): 224.0 [M+H]⁺.

### Step 5: Synthesis of Compound 25

Compound 25 (23 mg) was obtained through synthesis and purification using a method identical to that described in step 5 of Example 23, with compound 25e (45 mg, 0.2 mmol) and compound 4g (94 mg, 0.2 mmol) as starting materials.

MS m/z (ESI): 560.2 [M+H]⁺.

¹H NMR (400 MHz, DMSO-d₆) δ 12.48 (s, 1H), 10.16 (s, 1H), 8.34 (d, J=7.6 Hz, 1H), 8.04 (d, J=8.6 Hz, 1H), 7.33-7.22 (m, 3H), 7.10 (dd, J=8.6, 2.1 Hz, 1H), 4.70 (t, J=12.5 Hz, 2H), 3.76 (td, J=6.4, 2.2 Hz, 2H), 3.39-3.34 (m, 2H), 3.22 (t, J=6.9 Hz, 2H), 3.04-2.96 (m, 2H), 1.91 (m, 2H), 1.81 (m, 2H), 1.80 (br, 4H), 0.32 (s, 4H).

### Example 26: Synthesis of Compound 26

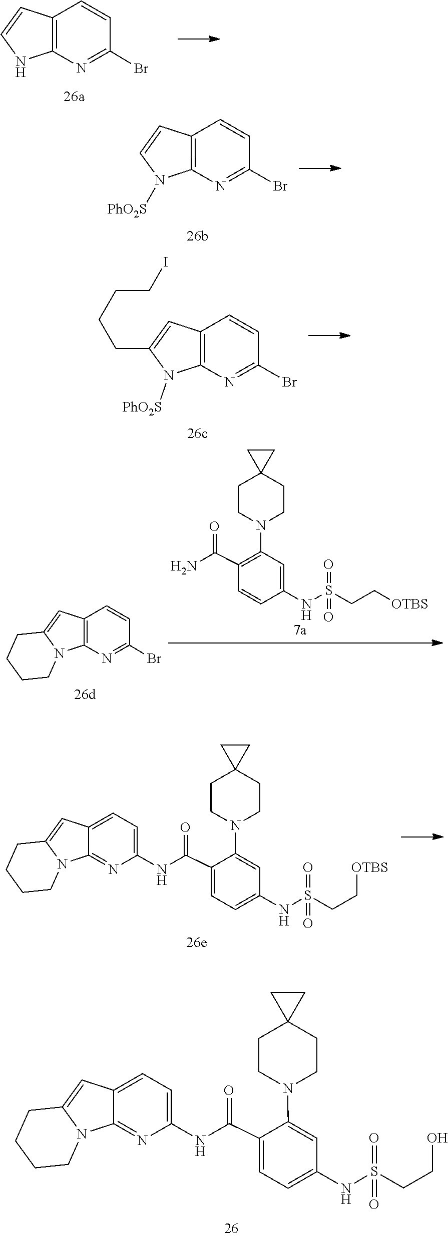

### Step 1: Synthesis of Compound 26b

Sodium hydride (2.2 g, 55.8 mmol, 60% dispersion in mineral oil) was added in batches to a solution of compound 26a (10.0 g, 50.8 mmol) in THE (100 mL) at 0 °C, and the mixture was stirred and reacted sufficiently at 0 °C. A solution of benzenesulfonyl chloride (9.9 g, 55.8 mmol) in THE (20 mL) was added dropwise to the reaction mixture, and the mixture was reacted sufficiently at room temperature. The reaction mixture was poured into a mixture of ethyl acetate (50 mL) and a saturated sodium bicarbonate solution (50 mL), and ethyl acetate (50 mL×3) was added for extraction. The combined organic phases were dried over anhydrous sodium sulfate and filtered, and the solvent was removed under reduced pressure. The residue was slurried with methyl tert-butyl ether (50 mL) and filtered to give compound 26b (14.5 g).

### Step 2: Synthesis of Compound 26c

Lithium diisopropylamide (2 M in THF, 9.6 mL) was slowly added dropwise to a solution of compound 26b (5.0 g, 14.8 mmol) and tetramethylethylenediamine (3.5 g, 29.7 mmol, 4.4 mL) in THE (100 mL) at −78 °C, and then the mixture was held at −78 °C and 1,4-diiodobutane (5.1 g, 16.3 mmol) was slowly added dropwise. The mixture was warmed to room temperature and reacted sufficiently. A saturated ammonium chloride solution (50 mL) was added to the reaction mixture to quench the reaction, and dichloromethane (50 mL×3) was added for extraction. The combined organic phases were dried over anhydrous sodium sulfate and filtered, and the filtrate was concentrated to give the crude product of compound 26c (9.0 g), which was used directly in the next step.

### Step 3: Synthesis of Compound 26d

Sodium tert-butoxide (3.3 g, 34.7 mmol) was added to a solution of compound 26c (9.0 g, 17.3 mmol) in 1,4-dioxane (150 mL). The mixture was stirred and reacted sufficiently at 80 °C, and the solvent was removed under reduced pressure. The residue was subjected to flash column chromatography (petroleum ether:ethyl acetate=100:0-85:15) to give compound 26d (650 mg).

¹H NMR (400 MHz, DMSO-d₆) δ 7.79 (d, J=8.1 Hz, 1H), 7.20 (d, J=8.1 Hz, 1H), 6.22 (d, J=1.3 Hz, 1H), 4.09 (t, J=6.2 Hz, 2H), 2.96-2.89 (m, 2H), 2.04-1.96 (m, 2H), 1.87-1.78 (m, 2H).

### Step 4: Synthesis of Compound 26e

Compound 26e (45 mg) was obtained through synthesis and purification using a method identical to that described in step 2 of Example 7, with compound 26d (43 mg, 0.17 mmol) and 7a (80 mg, 0.17 mmol) as starting materials.

MS m/z (ESI): 637.3 [M+H]⁺.

### Step 5: Synthesis of Compound 26

Compound 26 (19 mg) was obtained through synthesis and purification using a method identical to that described in step 8 of Example 4, with compound 26e (35 mg) as starting material.

MS m/z (ESI): 524.2 [M+H]⁺.

¹H NMR (400 MHz, DMSO-d₆) δ 13.36 (s, 1H), 10.19 (s, 1H), 8.08 (d, J=8.6 Hz, 1H), 8.03 (d, J=8.4 Hz, 1H), 7.85 (d, J=8.4 Hz, 1H), 7.30-7.24 (m, 1H), 7.12 (d, J=8.6 Hz, 1H), 6.14 (s, 1H), 4.96 (s, 1H), 4.09 (t, J=6.1 Hz, 2H), 3.76 (t, J=6.5 Hz, 2H), 3.38-3.35 (m, 2H), 3.07-2.95 (m, 4H), 2.93 (t, J=6.4 Hz, 2H), 2.10-2.01 (m, 2H), 2.00-1.62 (m, 6H), 0.41 (s, 4H).

### Example 27: Synthesis of Compound 27

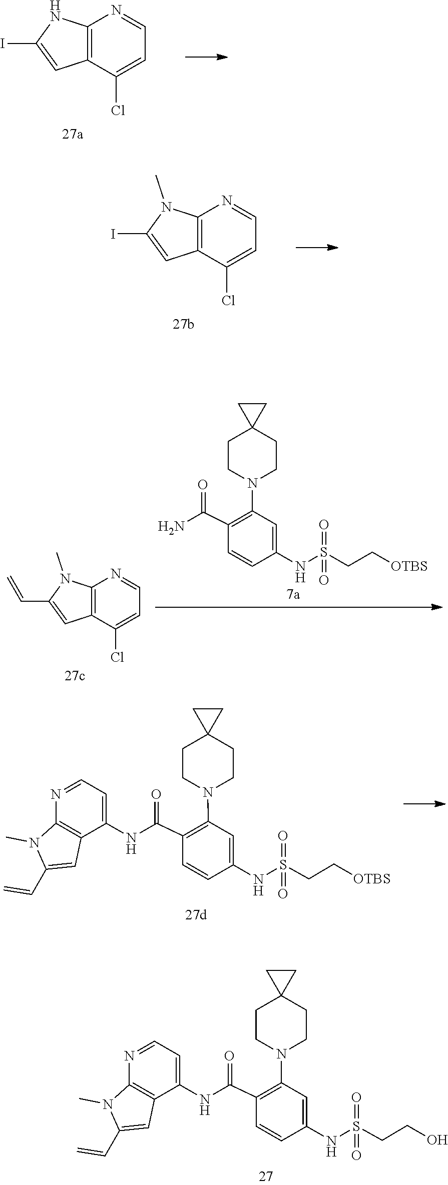 [SMILES: Clc1ccnc2[nH]c(I)cc12 ; Cn1c(I)cc2c(Cl)ccnc21 ; C=Cc1cc2c(Cl)ccnc2n1C ; CC(C)(C)[Si](C)(C)OCCS(=O)(=O)Nc1ccc(C(N)=O)c(N2CCC3(CC2)CC3)c1 ; C=Cc1cc2c(NC(=O)c3ccc(NS(=O)(=O)CCO[Si](C)(C)C(C)(C)C)cc3N3CCC4(CC3)CC4)ccnc2n1C ; C=Cc1cc2c(NC(=O)c3ccc(NS(=O)(=O)CCO)cc3N3CCC4(CC3)CC4)ccnc2n1C]

### Step 1: Synthesis of Compound 27b

Compound 27a (200 mg, 0.72 mmol) and cesium carbonate (701 mg, 2.16 mmol) were added to DMF (2 mL), followed by the addition of methyl iodide (306 mg, 2.16 mmol) at 0 °C. The mixture was reacted sufficiently at room temperature. A saturated sodium bicarbonate solution (5 mL) was added to the system, and dichloromethane (3 mL×3) was added for extraction. The combined organic phases were washed with saturated brine, dried over anhydrous magnesium sulfate, filtered, and concentrated to dryness by rotary evaporation, and the residue was purified by flash column chromatography (petroleum ether:ethyl acetate=5:1) to give compound 27b (180 mg).

MS m/z (ESI): 293.2 [M+H]⁺.

### Step 2: Synthesis of Compound 27c

In an argon atmosphere, compound 27b (180 mg, 0.62 mmol), potassium vinyltetrafluoroborate (165 mg, 1.24 mmol), and cesium carbonate (400 mg, 1.23 mmol) were added to dioxane (2 mL), followed by the addition of Pd(dppf)Cl₂ (45.0 mg, 62 μmol). The mixture was reacted sufficiently at 50 °C. After being cooled to room temperature, the reaction mixture was filtered and concentrated, and the residue was purified by flash column chromatography (petroleum ether:ethyl acetate=3:1) to give compound 27c (66 mg).

MS m/z (ESI): 193.2 [M+H]⁺.

### Step 3: Synthesis of Compound 27d

Compound 27d (6 mg) was obtained through synthesis and purification using a method identical to that described in step 2 of Example 7, with compound 27c (12 mg, 64 μmol) and 7a (20 mg, 43 μmol) as starting materials.

MS m/z (ESI): 624.3 [M+H]⁺.

### Step 4: Synthesis of Compound 27

Compound 27 (2.6 mg) was obtained through synthesis and purification using a method identical to that described in step 8 of Example 4, with compound 27d (6 mg) as starting material.

MS m/z (ESI): 510.2 [M+H]⁺.

### Example 28: Synthesis of Compound 28

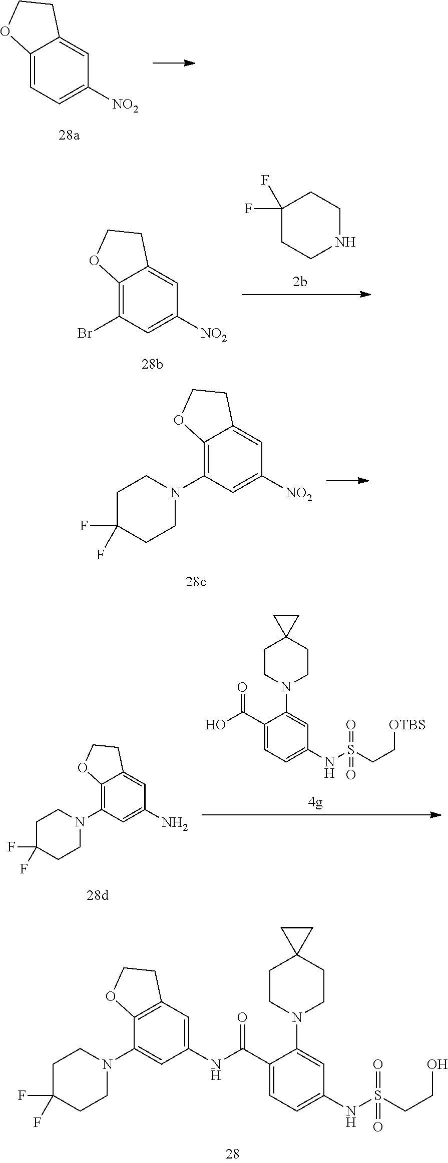

### Step 1: Synthesis of Compound 28b

Compound 28a (1.0 g, 6 mmol) was dissolved in 1,4-dioxane (10 mL), followed by the addition of liquid bromine (3.2 g, 20 mmol). The mixture was reacted sufficiently at room temperature. A saturated sodium bicarbonate solution (10 mL) and a sodium thiosulfate solution (10 mL) were added to the system, and dichloromethane (10 mL×3) was added for extraction. The combined organic phases were washed with saturated brine, dried over anhydrous magnesium sulfate, and filtered, and the filtrate was concentrated to dryness by rotary evaporation. The residue was purified by flash column chromatography (petroleum ether:ethyl acetate=8:1) to give compound 28b (890 mg).

### Step 2: Synthesis of Compound 28c

Compound 28c (278 mg) was obtained through synthesis and purification using a method identical to that described in step 4 of Example 22, with compound 28b (600 mg, 2.5 mmol) and 2b (hydrochloride, 790 mg, 5 mmol) as starting materials.

### Step 3: Synthesis of Compound 28d

Compound 28d (110 mg) was obtained through synthesis and purification using a method identical to that described in step 3 of Example 4, with compound 28c (200 mg, 0.70 mmol) as starting material.

MS m/z (ESI): 255.1 [M+H]⁺.

### Step 4: Synthesis of Compound 28

Compound 28 (27 mg) was obtained through synthesis and purification using a method identical to that described in step 5 of Example 23, with compound 28d (51 mg, 0.2 mmol) and compound 4g (94 mg, 0.2 mmol) as starting materials.

MS m/z (ESI): 591.2 [M+H]⁺.

¹H NMR (400 MHz, DMSO-d₆) δ 11.52 (s, 1H), 10.06 (s, 1H), 7.81 (d, J=9.2 Hz, 1H), 7.41 (s, 1H), 7.15 (s, 1H), 6.98-7.06 (m, 2H), 4.52 (t, J=8.6 Hz, 2H), 3.75 (t, J=6.2 Hz, 2H), 3.24-3.12 (m, 6H), 2.95 (br, 4H), 2.16-1.97 (m, 6H), 1.54 (br, 4H), 0.34 (s, 4H).

### Example 29: Synthesis of Compound 29

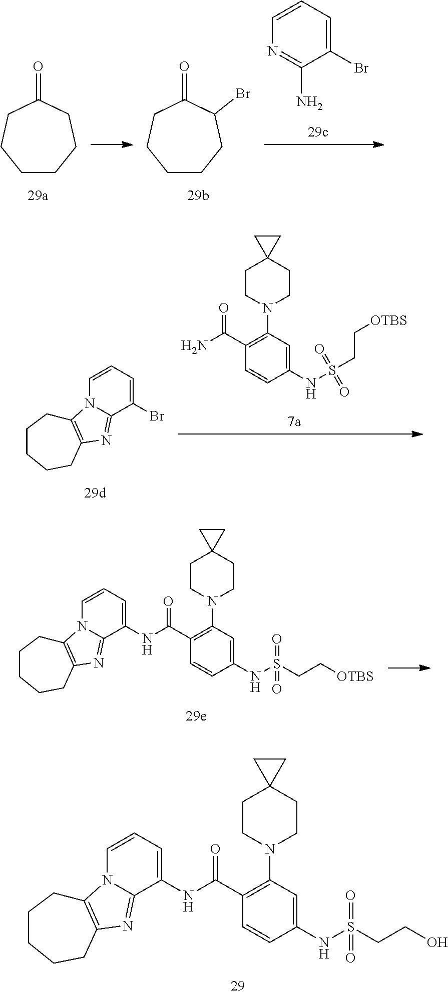

### Step 1: Synthesis of Compound 29b

NBS (433 mg, 2.4 mmol) and p-toluenesulfonic acid monohydrate (35 mg, 0.20 mmol) were added to dichloromethane (2 mL), followed by the addition of compound 29a (200 mg, 2.0 mmol) at 0 °C. The mixture was reacted sufficiently at 0 °C. A saturated sodium bicarbonate solution (3 mL) was added to the system, and dichloromethane (2 mL×3) was added for extraction. The combined organic phases were washed with saturated brine, dried over anhydrous magnesium sulfate, and filtered, and the filtrate was concentrated to dryness by rotary evaporation. The residue was purified by flash column chromatography (petroleum ether:ethyl acetate=10:1) to give compound 29b (286 mg).

### Step 2: Synthesis of Compound 29d

Compound 29b (100 mg, 0.57 μmol) and compound 29c (98 mg, 0.57 μmol) were added to ethanol (2 mL), and the mixture was reacted sufficiently at 80 °C. The reaction mixture was filtered and purified by reverse-phase flash column chromatography (acetonitrile:water=95:5-10:90) to give compound 29d (50 mg).

MS m/z (ESI): 265.0 [M+H]⁺.

### Step 3: Synthesis of Compound 29e

Compound 29e (9 mg) was obtained through synthesis and purification using a method identical to that described in step 2 of Example 7, with compound 29d (10 mg, 38 μmol) and 7a (35 mg, 75 μmol) as starting materials.

MS m/z (ESI): 652.3 [M+H]⁺.

### Step 4: Synthesis of Compound 29

Compound 29 (4 mg) was obtained through synthesis and purification using a method identical to that described in step 8 of Example 4, with compound 29e (9 mg) as starting material.

MS m/z (ESI): 538.2 [M+H]⁺.

### Example 30: Synthesis of Compound 30

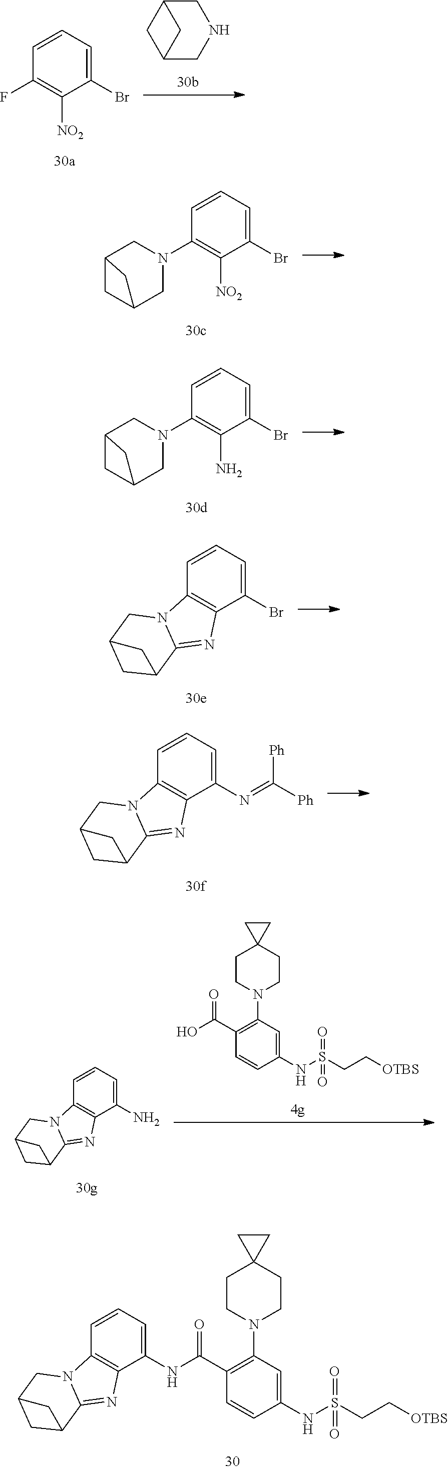

### Step 1: Synthesis of Compound 30c

Compound 30a (100 mg, 0.46 mmol), compound 30b (hydrochloride, 73 mg, 0.55 mmol), and cesium carbonate (371 mg, 1.14 mmol) were added to acetonitrile (1 mL), and the mixture was stirred and reacted sufficiently at 60 °C. Water (5 mL) was added to the system, and ethyl acetate (5 mL×3) was added for extraction. The combined organic phases were washed with saturated brine, dried over anhydrous magnesium sulfate, filtered, and concentrated to dryness by rotary evaporation, and the residue was purified by flash column chromatography (petroleum ether:ethyl acetate=5:1) to give compound 30c (125 mg).

MS m/z (ESI): 297.2 [M+H]⁺.

### Step 2: Synthesis of Compound 30d

Compound 30d (90 mg) was obtained through synthesis and purification using a method identical to that described in step 3 of Example 4, with compound 30c (125 mg, 0.42 mmol) as starting material.

MS m/z (ESI): 267.1 [M+H]⁺.

### Step 3: Synthesis of Compound 30e

Compound 30d (90 mg, 0.34 mmol) was added to acetonitrile (1 mL) and water (0.1 mL), followed by the addition of iodine (430 mg, 1.69 mmol) at 80 °C. The mixture was stirred and reacted sufficiently at that temperature. After the reaction mixture was cooled to room temperature, ethyl acetate (5 mL) was added to the system, followed by washing with a saturated aqueous sodium carbonate solution (5 mL) and a sodium thiosulfate solution (5 mL). The collected organic phase was washed with saturated brine, dried over anhydrous magnesium sulfate, filtered, and concentrated to dryness by rotary evaporation, and the residue was purified by flash column chromatography (petroleum ether:ethyl acetate=3:1) to give compound 30e (76 mg).

MS m/z (ESI): 263.2 [M+H]⁺.

### Step 4: Synthesis of Compound 30f

Compound 30e (76 mg, 0.29 mmol), benzophenone imine (105 mg, 0.58 mmol), and cesium carbonate (291 mg, 0.87 mmol) were added to 1,4-dioxane (1 mL), followed by the addition of XantPhos (33.5 mg, 58 μmol) and Pd₂(dba)₃ (26.5 mg, 29 μmol). The mixture was reacted sufficiently at 100 °C. The reaction mixture was filtered and concentrated to dryness by rotary evaporation, and the residue was purified by flash column chromatography (petroleum ether:ethyl acetate=1:1) to give compound 30f (80 mg).

MS m/z (ESI): 364.3 [M+H]⁺.

### Step 5: Synthesis of Compound 30g

Compound 30f (80 mg, 22 μmol) was dissolved in 1,4-dioxane (1 mL), followed by the addition of a solution of hydrogen chloride in dioxane (4 N, 1.8 mL, 4.5 mmol). The mixture was reacted sufficiently at room temperature. The reaction mixture was filtered, and the filter cake was collected and dried to give compound 30g (43 mg).

MS m/z (ESI): 200.1 [M+H]⁺.

### Step 6: Synthesis of Compound 30

Compound 30 (11 mg) was obtained through synthesis and purification using a method identical to that described in step 5 of Example 23, with compound 30g (20 mg, 0.1 mmol) and compound 4g (47 mg, 0.1 mmol) as starting materials.

MS m/z (ESI): 536.2 [M+H]⁺.

¹H NMR (400 MHz, DMSO-d₆) δ 10.16 (s, 1H), 8.12 (s, 1H), 8.00 (d, J=8.5 Hz, 1H), 7.41-7.19 (m, 3H), 7.08 (dd, J=8.5, 2.0 Hz, 1H), 4.37 (s, 2H), 3.76 (t, J=6.5 Hz, 2H), 3.35 (t, J=6.5 Hz, 2H), 3.11-2.87 (m, 6H), 2.74 (dt, J=8.3, 5.4 Hz, 2H), 1.85 (s, 2H), 1.70 (s, 2H), 1.24 (s, 2H), 0.28 (s, 4H).

### Example 31: Synthesis of Compound 31

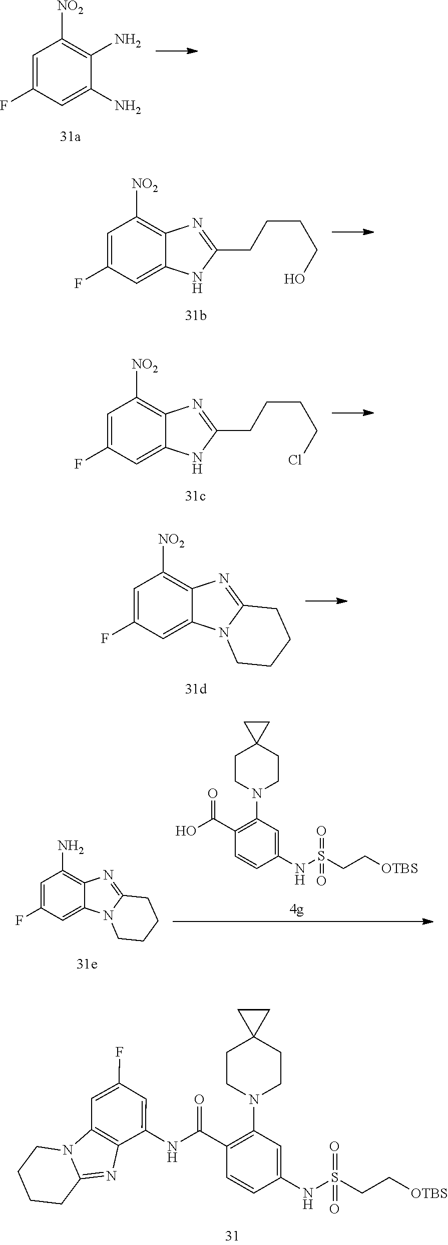

### Step 1: Synthesis of Compound 31b

Compound 31b (470 mg) was obtained through synthesis and purification using a method identical to that described in step 1 of Example 12, with compound 31a (500 mg, 2.92 mmol) and 5-valerolactone (500 mg, 5 mmol) as starting materials.

MS m/z (ESI): 254.0 [M+H]⁺.

### Step 2: Synthesis of Compound 31c

Compound 31c (283 mg) was obtained through synthesis and purification using a method identical to that described in step 2 of Example 12, with compound 31b (350 mg, 1.38 mmol) as starting material.

MS m/z (ESI): 272.0 [M+H]⁺.

### Step 3: Synthesis of Compound 31d

Compound 31d (135 mg) was obtained through synthesis and purification using a method identical to that described in step 3 of Example 12, with compound 31c (200 mg, 0.74 mmol) as starting material.

MS m/z (ESI): 237.0 [M+H]⁺.

### Step 4: Synthesis of Compound 31e

Compound 31e (77 mg) was obtained through synthesis and purification using a method identical to that described in step 3 of Example 4, with compound 31d (110 mg, 0.46 mmol) as starting material.

MS m/z (ESI): 206.1 [M+H]⁺.

### Step 5: Synthesis of Compound 31

Compound 31 (19 mg) was obtained through synthesis and purification using a method identical to that described in step 5 of Example 23, with compound 31e (41 mg, 0.2 mmol) and compound 4g (94 mg, 0.2 mmol) as starting materials.

MS m/z (ESI): 542.2 [M+H]⁺.

¹H NMR (400 MHz, DMSO-d₆) δ 12.08 (s, 1H), 10.17 (s, 1H), 7.95 (d, J=8.5 Hz, 1H), 7.85 (br, 1H), 7.45 (br, 1H), 7.24 (d, J=2.1 Hz, 1H), 7.08 (dd, J=8.5, 2.1 Hz, 1H), 4.31 (br, 2H), 3.77 (t, J=6.5 Hz, 2H), 3.36 (t, J=6.5 Hz, 2H), 3.21 (br, 4H), 3.03 (br, 4H), 2.82-2.69 (m, 2H), 1.57 (br, 4H), 0.31 (s, 4H).

### Example 32: Synthesis of Compound 32

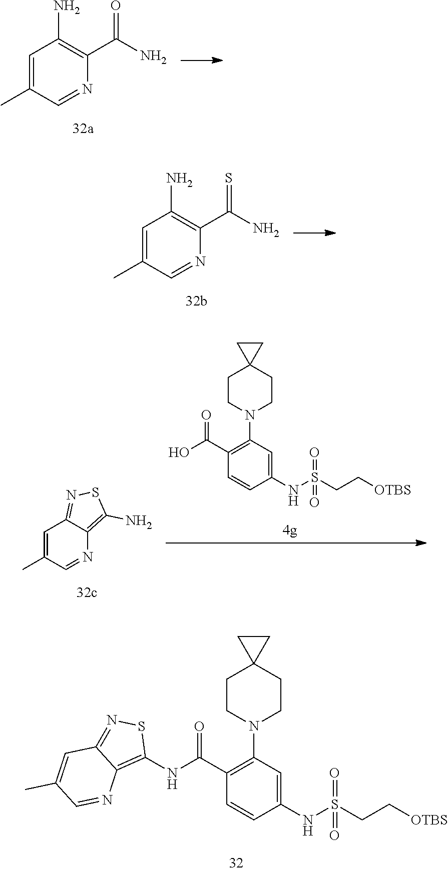 [SMILES: Cc1cnc(C(N)=O)c(N)c1 ; Cc1cnc(C(N)=S)c(N)c1 ; Cc1cnc2c(N)snc2c1 ; CC(C)(C)[Si](C)(C)OCCS(=O)(=O)Nc1ccc(C(=O)O)c(N2CCC3(CC2)CC3)c1 ; Cc1cnc2c(NC(=O)c3ccc(NS(=O)(=O)CCO[Si](C)(C)C(C)(C)C)cc3N3CCC4(CC3)CC4)snc2c1]

### Step 1: Synthesis of Compound 32b

Compound 32a (500 mg, 3.31 mmol) was added to anhydrous ethanol (10 mL), followed by the addition of phosphorus pentasulfide (1.0 mL, 4.5 mmol) in batches. The mixture was stirred and reacted sufficiently at 80 °C. The reaction mixture was concentrated, and then the residue was purified by flash column chromatography (petroleum ether:ethyl acetate=2:1) to give compound 32b (320 mg).

MS m/z (ESI): 168.1 [M+H]⁺.

### Step 2: Synthesis of Compound 32c

Compound 32b (300 mg, 1.78 mmol) was dissolved in methanol (10 mL), followed by the slow and dropwise addition of 30% hydrogen peroxide. The mixture was monitored until the reaction was sufficient. The reaction mixture was concentrated, and then the residue was purified by flash column chromatography (petroleum ether:ethyl acetate=3:1) to give compound 32c (93 mg).

MS m/z (ESI): 166.0 [M+H]⁺.

### Step 3: Synthesis of Compound 32

Compound 32 (3.5 mg) was obtained through synthesis and purification using a method identical to that described in step 5 of Example 23, with compound 32c (33 mg, 0.2 mmol) and compound 4g (94 mg, 0.2 mmol) as starting materials.

MS m/z (ESI): 502.2 [M+H]⁺.

### Example 33: Synthesis of Compound 33

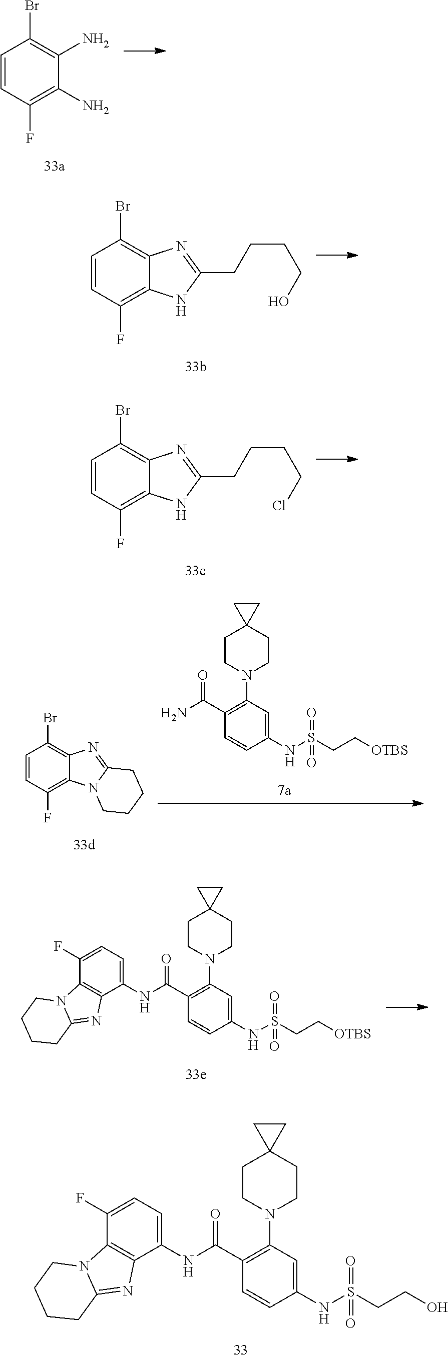

### Step 1: Synthesis of Compound 33b

Compound 33b (427 mg) was obtained through synthesis and purification using a method identical to that described in step 1 of Example 12, with compound 33a (500 mg, 2.45 mmol) and 5-valerolactone (500 mg, 5 mmol) as starting materials.

MS m/z (ESI): 287.0 [M+H]⁺.

### Step 2: Synthesis of Compound 33c

Compound 33c (190 mg) was obtained through synthesis and purification using a method identical to that described in step 2 of Example 12, with compound 33b (300 mg, 1.05 mmol) as starting material.

MS m/z (ESI): 305.0 [M+H]⁺.

### Step 3: Synthesis of Compound 33d

Compound 33d (35 mg) was obtained through synthesis and purification using a method identical to that described in step 3 of Example 12, with compound 33c (180 mg, 0.74 mmol) as starting material.

MS m/z (ESI): 269.1 [M+H]⁺.

### Step 4: Synthesis of Compound 33e

Compound 33e (29 mg) was obtained through synthesis and purification using a method identical to that described in step 2 of Example 7, with compound 33d (30 mg, 0.11 mmol) and 7a (52 mg, 0.11 mmol) as starting materials.

MS m/z (ESI): 665.3 [M+H]⁺.

### Step 5: Synthesis of Compound 33

Compound 33 (13 mg) was obtained through synthesis and purification using a method identical to that described in step 8 of Example 4, with compound 33e (29 mg) as starting material.

MS m/z (ESI): 542.2 [M+H]⁺.

### Example 34: Synthesis of Compound 34

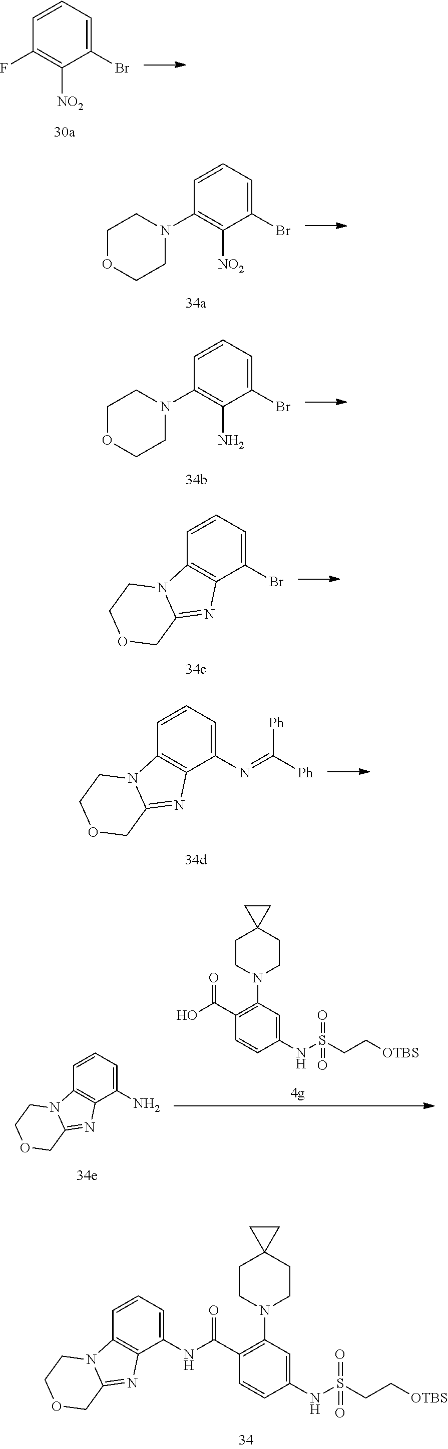

### Step 1: Synthesis of Compound 34a

Compound 34a (200 mg) was obtained through synthesis and purification using a method identical to that described in step 1 of Example 30, with compound 30a (220 mg, 1.0 mmol) and morpholine as starting materials.

### Step 2: Synthesis of Compound 34b

Compound 34b (147 mg) was obtained through synthesis and purification using a method identical to that described in step 3 of Example 4, with compound 34a (200 mg, 0.70 mmol) as starting material.

MS m/z (ESI): 257.0 [M+H]⁺.

### Step 3: Synthesis of Compound 34c

Compound 34c (65 mg) was obtained through synthesis and purification using a method identical to that described in step 3 of Example 30, with compound 34b (100 mg, 0.39 mmol) as starting material.

MS m/z (ESI): 253.0 [M+H]⁺.

### Step 4: Synthesis of Compound 34d

Compound 34d (71 mg) was obtained through synthesis and purification using a method identical to that described in step 4 of Example 30, with compound 34c (65 mg, 0.26 mmol) as starting material.

MS m/z (ESI): 354.0 [M+H]⁺.

### Step 5: Synthesis of Compound 34e

Compound 34e (32 mg) was obtained through synthesis and purification using a method identical to that described in step 5 of Example 30, with compound 34d (71 mg, 0.20 mmol) as starting material.

MS m/z (ESI): 190.0 [M+H]⁺.

### Step 6: Synthesis of Compound 34

Compound 34 (17 mg) was obtained through synthesis and purification using a method identical to that described in step 5 of Example 23, with compound 34e (30 mg, 0.16 mmol) and compound 4g (75 mg, 0.16 mmol) as starting materials.

MS m/z (ESI): 526.2 [M+H]⁺.

### Example 35: Synthesis of Compound 35

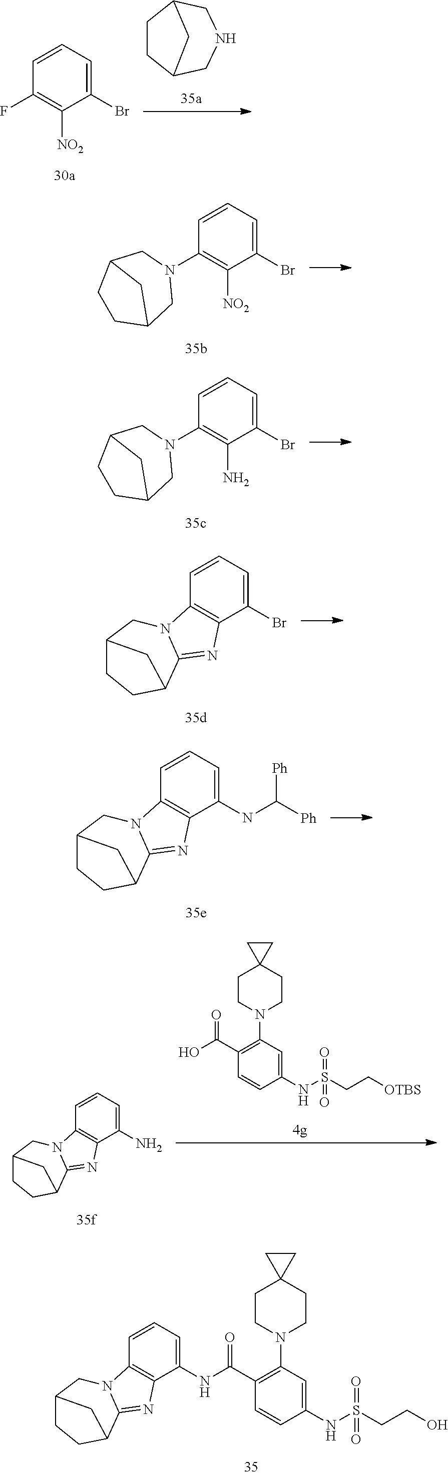

### Step 1: Synthesis of Compound 35b

Compound 35b (128 mg) was obtained through synthesis and purification using a method identical to that described in step 1 of Example 30, with compound 30a (100 mg, 0.46 mmol) and 35a (hydrochloride, 81 mg, 0.55 mmol) as starting materials.

### Step 2: Synthesis of Compound 35c

Compound 35c (95 mg) was obtained through synthesis and purification using a method identical to that described in step 3 of Example 4, with compound 35b (122 mg, 0.39 mmol) as starting material.

MS m/z (ESI): 281.0 [M+H]⁺.

### Step 3: Synthesis of Compound 35d

Compound 35d (73 mg) was obtained through synthesis and purification using a method identical to that described in step 3 of Example 30, with compound 35c (95 mg, 0.34 mmol) as starting material.

MS m/z (ESI): 277.0 [M+H]⁺.

### Step 4: Synthesis of Compound 35e

Compound 35e (66 mg) was obtained through synthesis and purification using a method identical to that described in step 4 of Example 30, with compound 35d (73 mg, 0.26 mmol) as starting material.

MS m/z (ESI): 378.1 [M+H]⁺.

### Step 5: Synthesis of Compound 35f

Compound 35f (37 mg) was obtained through synthesis and purification using a method identical to that described in step 5 of Example 30, with compound 35e (64 mg, 0.17 mmol) as starting material.

MS m/z (ESI): 214.1 [M+H]⁺.

### Step 6: Synthesis of Compound 35

Compound 35 (11 mg) was obtained through synthesis and purification using a method identical to that described in step 5 of Example 23, with compound 35f (32 mg, 0.15 mmol) and compound 4g (70 mg, 0.15 mmol) as starting materials.

MS m/z (ESI): 550.3 [M+H]⁺.

¹H NMR (400 MHz, DMSO-d₆) δ 12.45 (s, 1H), 8.32 (d, J=7.4 Hz, 1H), 8.02 (d, J=8.6 Hz, 1H), 7.25 (d, J=2.1 Hz, 1H), 7.18-7.02 (m, 3H), 4.14 (dd, J=11.5, 3.7 Hz, 1H), 3.96 (d, J=11.5 Hz, 1H), 3.76 (t, J=6.5 Hz, 2H), 3.43 (t, J=4.6 Hz, 2H), 3.00 (d, J=10.0 Hz, 4H), 2.87 (d, J=8.0 Hz, 1H), 2.15-1.96 (m, 4H), 1.94-1.85 (m, 5H), 1.68-1.53 (m, 2H), 0.30 (s, 4H).

### Example 36: Synthesis of Compound 36

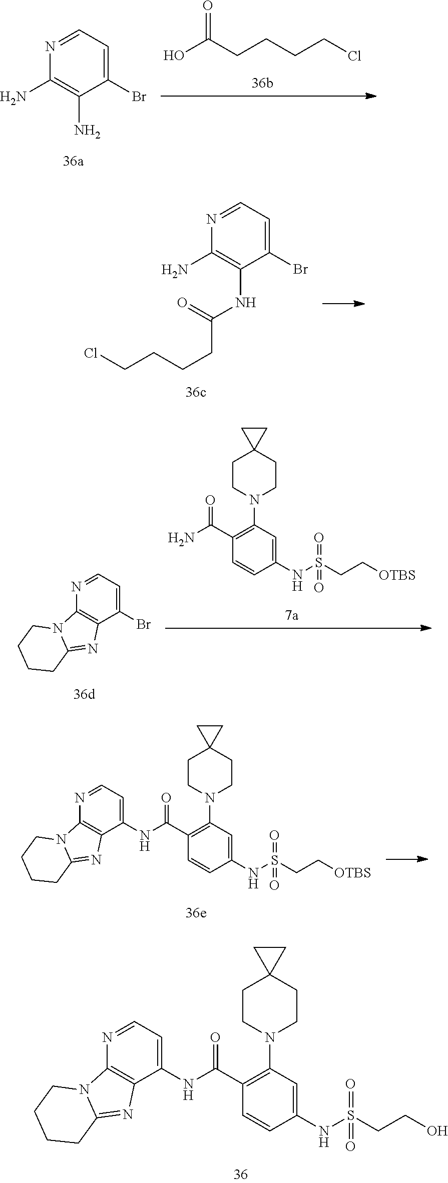

### Step 1: Synthesis of Compound 36c

Compound 36b (217 mg, 1.59 mmol), DIEA (617 mg, 4.78 mmol), and HATU (1.21 g, 3.18 mmol) were added to THE (3 mL), followed by the addition of compound 36a (300 mg, 1.59 mmol). The mixture was reacted sufficiently at room temperature. The reaction mixture was concentrated to dryness by rotary evaporation, and the residue was purified by flash column chromatography (petroleum ether:ethyl acetate=1:1) to give compound 36c (425 mg).

MS m/z (ESI): 306.2/308.2 [M+H]⁺.

### Step 2: Synthesis of Compound 36d

Compound 36c (425 mg, 1.39 mmol) was added to anhydrous acetic acid (4 mL), and the mixture was reacted sufficiently at 120 °C. The reaction mixture was concentrated to dryness by rotary evaporation, and the residue was purified by flash column chromatography (petroleum ether:ethyl acetate=2:1) to give compound 36d (181 mg).

MS m/z (ESI): 252.3/254.3 [M+H]⁺.

### Step 3: Synthesis of Compound 36e

Compound 36e (15 mg) was obtained through synthesis and purification using a method identical to that described in step 2 of Example 7, with compound 36d (16 mg, 0.064 mmol) and 7a (20 mg, 0.043 mmol) as starting materials.

MS m/z (ESI): 639.0 [M+H]⁺.

### Step 4: Synthesis of Compound 36

Compound 36 (3.6 mg) was obtained through synthesis and purification using a method identical to that described in step 8 of Example 4, with compound 36e (15 mg) as starting material.

MS m/z (ESI): 525.3 [M+H]⁺.

¹H NMR (400 MHz, DMSO-d₆) δ 12.37 (s, 1H), 10.15 (s, 1H), 8.33 (d, J=7.3 Hz, 1H), 8.06 (d, J=8.6 Hz, 1H), 7.34 (d, J=7.3 Hz, 1H), 7.29 (d, J=2.1 Hz, 1H), 7.10 (dd, J=8.6, 2.1 Hz, 1H), 4.94 (s, 1H), 4.11 (t, J=6.1 Hz, 2H), 4.06 (m, 4H), 3.76 (t, J=6.5 Hz, 2H), 3.36 (d, J=6.5 Hz, 2H), 3.00 (m, 2H), 2.62 (m, 4H), 1.85 (s, 4H), 0.35 (s, 4H).

### Example 39: Synthesis of Compound 39

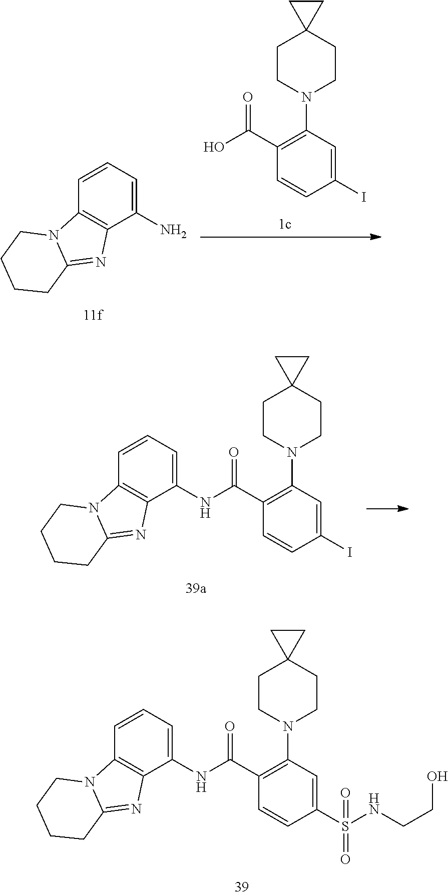 [SMILES: Nc1cccc2c1nc1n2CCCC1 ; O=C(O)c1ccc(I)cc1N1CCC2(CC1)CC2 ; O=C(Nc1cccc2c1nc1n2CCCC1)c1ccc(I)cc1N1CCC2(CC1)CC2 ; O=C(Nc1cccc2c1nc1n2CCCC1)c1ccc(S(=O)(=O)NCCO)cc1N1CCC2(CC1)CC2]

### Step 1: Synthesis of Compound 39a

Compound 39a (37 mg) was obtained through synthesis and purification using a method identical to that described in step 2 of Example 1, with compound 11f (30 mg, 0.16 mmol) and compound 1c (71 mg, 0.2 mol) as starting materials.

### Step 2: Synthesis of Compound 39

Compound 39a (30 mg, 57 μmol), DBU sulfur trioxide complex (24 mg, 0.1 mmol), and triethylamine (15 mg, 0.15 mmol) were added to isopropanol (0.5 mL), followed by the addition of palladium acetate (2.2 mg, 10 μmol) and XantPhos (5.8 mg, 10 μmol). The reaction mixture was reacted sufficiently at 80 °C in an argon atmosphere. After the reaction mixture was cooled to room temperature, ethanolamine (12 mg, 0.2 mmol) and sodium hypochlorite (10% aqueous solution, 150 mg, 0.2 mmol) were added. The mixture was reacted sufficiently at room temperature. The reaction mixture was directly purified by flash reverse-phase column chromatography (acetonitrile:water, 10-85%) to give compound 39 (4.5 mg).

MS m/z (ESI): 524.3 [M+H]⁺.

### Example 40: Synthesis of Compound 40

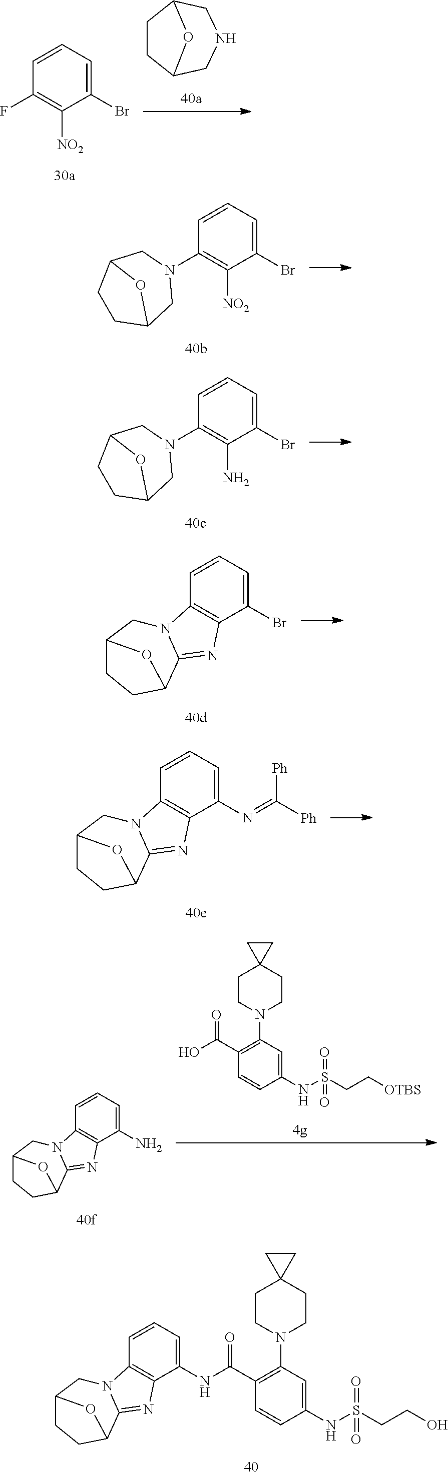

### Step 1: Synthesis of Compound 40b

Compound 40b (123 mg) was obtained through synthesis and purification using a method identical to that described in step 1 of Example 30, with compound 30a (100 mg, 0.46 mmol) and 40a (hydrochloride, 82 mg, 0.55 mmol) as starting materials.

### Step 2: Synthesis of Compound 40c

Compound 40c (90 mg) was obtained through synthesis and purification using a method identical to that described in step 3 of Example 4, with compound 40b (120 mg, 0.39 mmol) as starting material.

MS m/z (ESI): 281.3/283.3 [M+H]⁺.

### Step 3: Synthesis of Compound 40d

Compound 40d (72 mg) was obtained through synthesis and purification using a method identical to that described in step 3 of Example 30, with compound 40c (90 mg, 0.34 mmol) as starting material.

MS m/z (ESI): 277.2/279.2 [M+H]⁺.

### Step 4: Synthesis of Compound 40e

Compound 40e (62 mg) was obtained through synthesis and purification using a method identical to that described in step 4 of Example 30, with compound 40d (72 mg, 0.26 mmol) as starting material.

MS m/z (ESI): 380.1 [M+H]⁺.

### Step 5: Synthesis of Compound 40f

Compound 40f (35 mg) was obtained through synthesis and purification using a method identical to that described in step 5 of Example 30, with compound 40e (62 mg, 0.16 mmol) as starting material.

MS m/z (ESI): 216.2 [M+H]⁺.

### Step 6: Synthesis of Compound 40

Compound 40 (10.5 mg) was obtained through synthesis and purification using a method identical to that described in step 5 of Example 23, with compound 40f (25 mg, 0.12 mmol) and compound 4g (55 mg, 0.12 mmol) as starting materials.

MS m/z (ESI): 552.4 [M+H]⁺.

¹H NMR (400 MHz, DMSO-d₆) δ 12.48 (s, 1H), 8.36 (dd, J=7.7, 1.2 Hz, 1H), 8.03 (d, J=8.6 Hz, 1H), 7.26 (d, J=2.1 Hz, 1H), 7.23-7.13 (m, 2H), 7.07 (dd, J=8.6, 2.0 Hz, 1H), 5.33 (d, J=5.9 Hz, 1H), 5.02 (d, J=5.7 Hz, 1H), 4.30 (dd, J=11.9, 4.2 Hz, 1H), 4.05 (d, J=11.9 Hz, 1H), 3.76 (t, J=6.5 Hz, 2H), 3.31 (t, J=6.5 Hz, 2H), 2.99 (m, 6H), 2.32-2.21 (m, 2H), 2.10 (t, J=10.7 Hz, 1H), 1.93 (t, J=10.8 Hz, 1H), 1.24 (s, 2H), 0.32 (s, 4H).

### Example 41: Synthesis of Compound 41

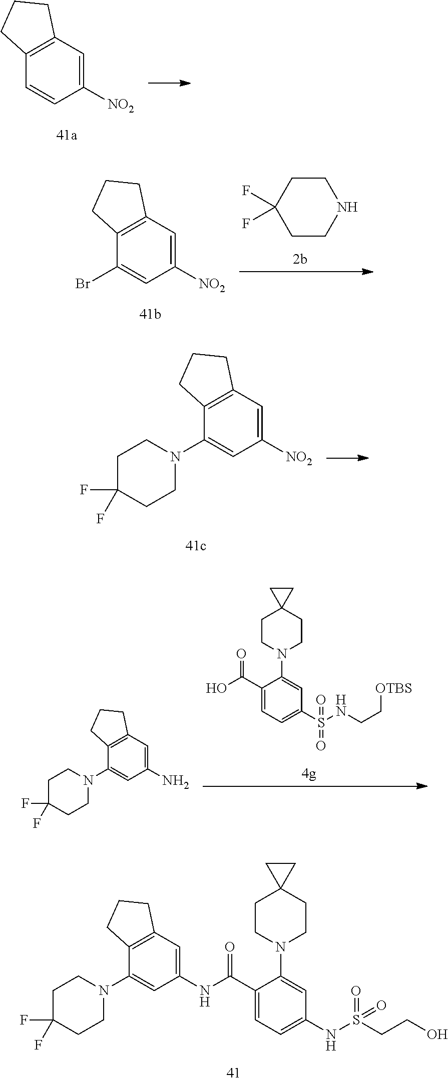

### Step 1: Synthesis of Compound 41b

Compound 41a (0.5 g, 3.1 mmol) and NBS (573 mg, 3.2 mmol) were added to concentrated sulfuric acid (2 mL), and the mixture was reacted sufficiently at 60 °C. After being cooled, the reaction mixture was slowly poured into ice water (40 mL), extracted with ethyl acetate (40 mL×3), and separated. The combined organic phases were dried over anhydrous sodium sulfate and concentrated to dryness by rotary evaporation, and the residue was purified by flash column chromatography (petroleum ether:ethyl acetate=12:1) to give compound 41b (500 mg).

### Step 2: Synthesis of Compound 41c

Compound 41c (99 mg) was obtained through synthesis and purification using a method identical to that described in step 4 of Example 22, with compound 41b (150 mg, 0.62 mmol) and 2b (hydrochloride, 146 mg, 0.93 mmol) as starting materials.

### Step 3: Synthesis of Compound 41d

Compound 41d (40 mg) was obtained through synthesis and purification using a method identical to that described in step 3 of Example 4, with compound 41c (80 mg, 0.14 mmol) as starting material.

### Step 4: Synthesis of Compound 41

Compound 41 (6 mg) was obtained through synthesis and purification using a method identical to that described in step 5 of Example 23, with compound 41d (36 mg, 0.14 mmol) and compound 4g (62 mg, 0.14 mmol) as starting materials.

MS m/z (ESI): 589.2 [M+H]⁺.

¹H NMR (400 MHz, DMSO-d₆) δ 11.52 (s, 1H), 10.06 (s, 1H), 7.81 (d, J=9.2 Hz, 1H), 7.41 (s, 1H), 7.15 (s, 1H), 6.98-7.06 (m, 2H), 4.52 (t, J=8.6 Hz, 2H), 3.75 (t, J=6.2 Hz, 2H), 3.24-3.12 (m, 6H), 2.95 (br, 4H), 2.16-1.97 (m, 6H), 1.54 (br, 4H), 0.34 (s, 4H).

### Example 42: Synthesis of Compound 42

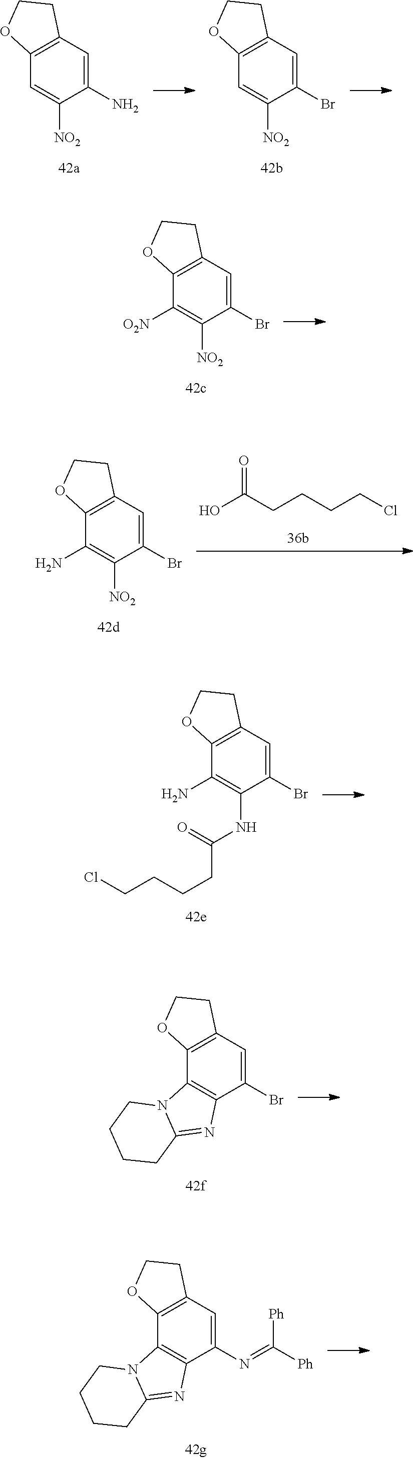

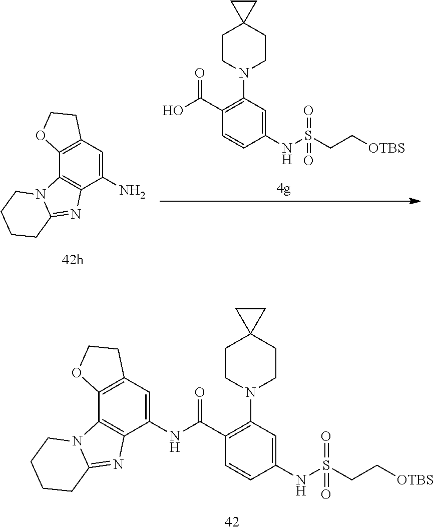 [SMILES: Nc1cc2c(c3c1nc1n3CCCC1)OCC2 ; CC(C)(C)[Si](C)(C)OCCS(=O)(=O)Nc1ccc(C(=O)O)c(N2CCC3(CC2)CC3)c1 ; CC(C)(C)[Si](C)(C)OCCS(=O)(=O)Nc1ccc(C(=O)Nc2cc3c(c4c2nc2n4CCCC2)OCC3)c(N2CCC3(CC2)CC3)c1]

### Step 1: Synthesis of Compound 42b

Compound 42a (300 mg, 1.66 mmol), tert-butyl nitrite (206 mg, 2.00 mmol), and cuprous bromide (357 mg, 2.50 mmol) were added to acetonitrile (3 mL), and the mixture was reacted sufficiently at 70 °C. Water (6 mL) was added to the system, and ethyl acetate (2 mL×3) was added for extraction. The combined organic phases were washed with saturated brine, dried over anhydrous magnesium sulfate, filtered, and concentrated to dryness by rotary evaporation, and the residue was purified by flash column chromatography (petroleum ether:ethyl acetate=5:1) to give compound 42b (320 mg).

### Step 2: Synthesis of Compound 42c

Compound 42b (150 mg, 0.62 mmol) was added to concentrated sulfuric acid (1 mL), followed by the addition of concentrated nitric acid (0.2 mL) at 0 °C. The mixture was reacted sufficiently at room temperature. The reaction mixture was poured into ice water (10 mL) and filtered, and the filter cake was purified by flash column chromatography (petroleum ether:ethyl acetate=4:1) to give compound 42c (68.0 mg).

### Step 3: Synthesis of Compound 42d

Compound 42d (43 mg) was obtained through synthesis and purification using a method identical to that described in step 3 of Example 4, with compound 42c (68 mg, 0.24 mmol) as starting material.

MS m/z (ESI): 229.3/231.3 [M+H]⁺.

### Step 4: Synthesis of Compound 42e

Compound 42e (57 mg) was obtained through synthesis and purification using a method identical to that described in step 1 of Example 36, with compound 42d (43 mg, 0.19 mmol) and 36b (26 mg, 0.19 mmol) as starting materials.

### Step 5: Synthesis of Compound 42f

Compound 42f (38 mg) was obtained through synthesis and purification using a method identical to that described in step 2 of Example 36, with compound 42e (55 mg, 0.16 mmol) as starting material.

MS m/z (ESI): 293.4/295.4 [M+H]⁺.

### Step 6: Synthesis of Compound 42g

Compound 42g (39 mg) was obtained through synthesis and purification using a method identical to that described in step 4 of Example 30, with compound 42f (38 mg, 0.13 mmol) as starting material.

MS m/z (ESI): 394.3 [M+H]⁺.

### Step 7: Synthesis of Compound 42h

Compound 42h (22 mg) was obtained through synthesis and purification using a method identical to that described in step 5 of Example 30, with compound 42g (39 mg, 0.1 mmol) as starting material.

MS m/z (ESI): 230.2 [M+H]⁺.

### Step 8: Synthesis of Compound 42

Compound 42 (3.9 mg) was obtained through synthesis and purification using a method identical to that described in step 5 of Example 23, with compound 42h (20 mg, 87 μmol) and compound 4g (47 mg, 0.1 mmol) as starting materials.

MS m/z (ESI): 566.3 [M+H]⁺.

¹H NMR (400 MHz, DMSO-d₆) δ 12.24 (s, 1H), 8.23 (s, 1H), 8.00 (d, J=8.6 Hz, 1H), 7.23 (d, J=2.1 Hz, 1H), 7.05 (dd, J=8.6, 2.1 Hz, 1H), 4.92 (s, 1H), 4.66 (t, J=8.7 Hz, 2H), 4.27 (t, J=5.8 Hz, 2H), 3.75 (t, J=6.5 Hz, 2H), 3.24 (t, J=6.5 Hz, 2H), 2.97 (m, 6H), 2.09-1.89 (m, 4H), 1.78 (m, 4H), 1.24 (m, 2H), 0.30 (s, 4H).

### Example 43: Synthesis of Compound 43

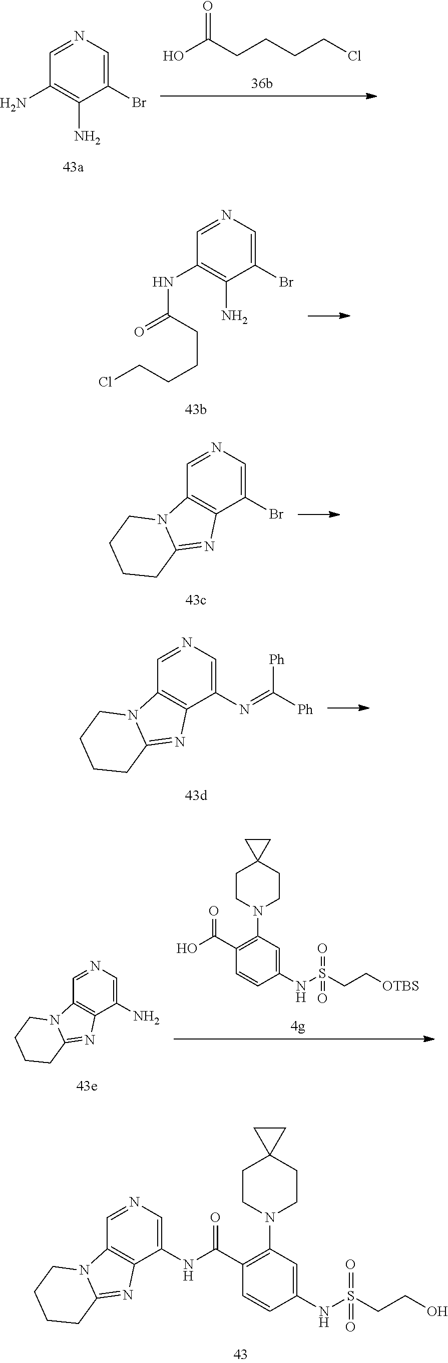

### Step 1: Synthesis of Compound 43b

Compound 43b (425 mg) was obtained through synthesis and purification using a method identical to that described in step 1 of Example 36, with compound 43a (217 mg, 1.59 mmol) and 36b (300 mg, 1.59 mmol) as starting materials.

### Step 2: Synthesis of Compound 43c

Compound 43c (143 mg) was obtained through synthesis and purification using a method identical to that described in step 2 of Example 36, with compound 43b (425 mg, 1.39 mmol) as starting material.

MS m/z (ESI): 252.2/254.2 [M+H]⁺.

### Step 3: Synthesis of Compound 43d

Compound 43d (140 mg) was obtained through synthesis and purification using a method identical to that described in step 4 of Example 30, with compound 43c (143 mg, 57 μmol) as starting material.

MS m/z (ESI): 353.2 [M+H]⁺.

### Step 4: Synthesis of Compound 43e

Compound 43e (62 mg) was obtained through synthesis and purification using a method identical to that described in step 5 of Example 30, with compound 43d (140 mg, 41.5 μmol) as starting material.

MS m/z (ESI): 189.2 [M+H]⁺.

### Step 5: Synthesis of Compound 43

Compound 43 (3.7 mg) was obtained through synthesis and purification using a method identical to that described in step 5 of Example 23, with compound 43e (19 mg, 0.1 mmol) and compound 4g (47 mg, 0.1 mmol) as starting materials.

MS m/z (ESI): 525.2 [M+H]⁺.

¹H NMR (400 MHz, DMSO-d₆) δ 12.68 (s, 1H), 9.47 (s, 1H), 8.59 (s, 1H), 8.08 (d, J=8.6 Hz, 1H), 7.30 (s, 1H), 7.11 (d, J=8.6 Hz, 1H), 4.93 (s, 1H), 4.25 (t, J=5.9 Hz, 2H), 3.76 (t, J=6.5 Hz, 2H), 3.28 (t, J=6.5 Hz, 2H), 3.05 (t, J=6.5 Hz, 2H), 2.99 (m, 4H), 2.13-1.95 (m, 4H), 1.84 (s, 4H), 0.33 (s, 4H).

### Example 44: Synthesis of Compound 44

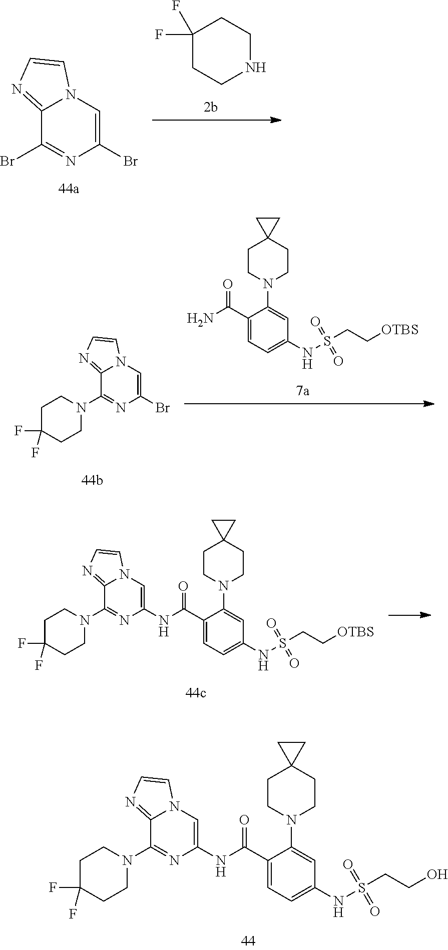 [SMILES: Brc1cn2ccnc2c(Br)n1 ; FC1(F)CCNCC1 ; FC1(F)CCN(c2nc(Br)cn3ccnc23)CC1 ; CC(C)(C)[Si](C)(C)OCCS(=O)(=O)Nc1ccc(C(N)=O)c(N2CCC3(CC2)CC3)c1 ; CC(C)(C)[Si](C)(C)OCCS(=O)(=O)Nc1ccc(C(=O)Nc2cn3ccnc3c(N3CCC(F)(F)CC3)n2)c(N2CCC3(CC2)CC3)c1 ; O=C(Nc1cn2ccnc2c(N2CCC(F)(F)CC2)n1)c1ccc(NS(=O)(=O)CCO)cc1N1CCC2(CC1)CC2]

### Step 1: Synthesis of Compound 44b

Compound 44a (300 mg, 1.08 mmol), compound 2b (hydrochloride, 189 mg, 1.2 mmol), and triethylamine (300 mg, 3 mmol) were added to acetonitrile (3 mL), and the mixture was reacted sufficiently at 40 °C. Water (20 mL) was added to the system, and ethyl acetate (10 mL×3) was added for extraction. The combined organic phases were washed with saturated brine, dried over anhydrous magnesium sulfate, filtered, and concentrated to dryness by rotary evaporation, and the residue was purified by flash column chromatography (petroleum ether:ethyl acetate=3:1) to give compound 44b (305 mg).

MS m/z (ESI): 317.1/319.1 [M+H]⁺.

### Step 2: Synthesis of Compound 44c

Compound 44c (40 mg) was obtained through synthesis and purification using a method identical to that described in step 2 of Example 7, with compound 44b (32 mg, 0.1 mmol) and 7a (47 mg, 0.1 mmol) as starting materials.

### Step 3: Synthesis of Compound 44

Compound 44 (28 mg) was obtained through synthesis and purification using a method identical to that described in step 8 of Example 4, with compound 44c (40 mg, 0.058 mmol) as starting material.

MS m/z (ESI): 590.3 [M+H]⁺.

¹H NMR (400 MHz, DMSO-d₆) δ 10.63 (s, 1H), 9.72 (s, 1H), 7.27 (s, 1H), 7.16 (d, J=8.1 Hz, 1H), 6.66-6.39 (m, 2H), 4.95 (s, 1H), 3.84 (s, 4H), 3.74 (t, J=6.8 Hz, 2H), 3.51-3.37 (m, 2H), 3.26 (t, J=6.8 Hz, 2H), 3.14 (d, J=10.0 Hz, 2H), 2.29 (s, 3H), 1.93 (d, J=14.5 Hz, 4H), 1.48 (m, 2H), 0.99 (s, 2H), 0.80 (s, 2H).

### Example 45: Synthesis of Compound 45

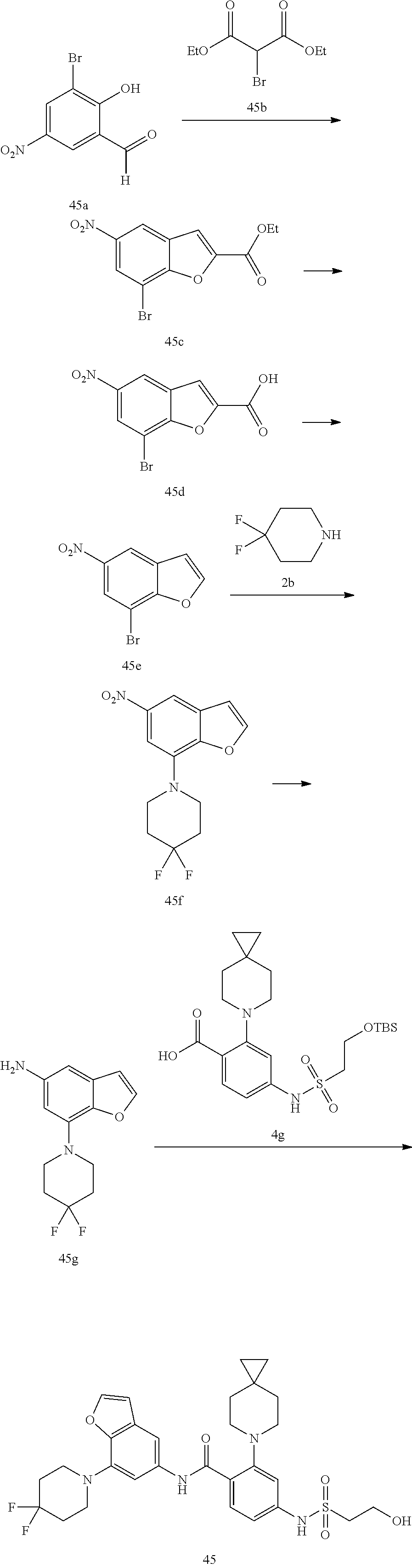

### Step 1: Synthesis of Compound 45c

Compound 45a (1.0 g, 4.06 mmol), compound 45b (1.17 g, 4.88 mmol), and potassium carbonate (1.69 g, 12.2 mmol) were added to acetone (20 mL), and the mixture was heated to 80 °C and reacted sufficiently. After being cooled to room temperature, the reaction mixture was poured into water (100 mL) and extracted with ethyl acetate (50 mL×3). The organic phases were combined, dried over anhydrous sodium sulfate, filtered, and concentrated to dryness by rotary evaporation, and the residue was purified by flash column chromatography (petroleum ether:ethyl acetate=4:1) to give compound 45c (520 mg).

### Step 2: Synthesis of Compound 45d

Sodium hydroxide (132 mg, 3.31 mmol) was added to a mixed solution of compound 45c (0.52 g, 1.66 mmol) in methanol (4 mL), tetrahydrofuran (8 mL), and water (2 mL), and the mixture was reacted sufficiently at room temperature. The reaction mixture was poured into water (50 mL), the pH was adjusted to 3 with diluted hydrochloric acid, and ethyl acetate (30 mL×3) was added for extraction. The combined organic phases were dried over anhydrous sodium sulfate, filtered, and concentrated to dryness by rotary evaporation, and the residue was purified by flash column chromatography (ethyl acetate) to give compound 45d (350 mg).

### Step 3: Synthesis of Compound 45e

Copper powder (133 mg, 2.10 mmol) was added to a solution of compound 45d (300 mg, 1.05 mmol) in quinoline (3 mL), and the mixture was heated to 200 °C and reacted sufficiently. After being cooled, the reaction mixture was poured into water (50 mL), the pH was adjusted to 6-7 with diluted hydrochloric acid, and ethyl acetate (20 mL×3) was added for extraction. The combined organic phases were dried over anhydrous sodium sulfate, filtered, and concentrated to dryness by rotary evaporation, and the residue was purified by flash column chromatography (petroleum ether:ethyl acetate=10:1) to give compound 45e (120 mg).

### Step 4: Synthesis of Compound 45f

Compound 45f (278 mg) was obtained through synthesis and purification using a method identical to that described in step 4 of Example 22, with compound 45e (150 mg, 0.62 mmol) and 2b (hydrochloride, 127 mg, 0.8 mmol) as starting materials.

### Step 5: Synthesis of Compound 45g

Compound 45g (70 mg) was obtained through synthesis and purification using a method identical to that described in step 3 of Example 4, with compound 45f (120 mg, 0.42 mmol) as starting material.

MS m/z (ESI): 253.1 [M+H]⁺.

### Step 6: Synthesis of Compound 45

Compound 45 (12 mg) was obtained through synthesis and purification using a method identical to that described in step 5 of Example 23, with compound 45g (70 mg, 0.28 mmol) and compound 4g (130 mg, 0.28 mmol) as starting materials.

MS m/z (ESI): 589.1 [M+H]⁺.

¹H NMR (400 MHz, DMSO-d₆) δ 11.71 (s, 1H), 7.96 (d, J=2.1 Hz, 1H), 7.87-7.82 (m, 2H), 7.17 (d, J=2.1 Hz, 1H), 7.09 (d, J=1.9 Hz, 1H), 7.04 (dd, J=8.5, 2.1 Hz, 1H), 6.95 (d, J=2.2 Hz, 1H), 3.76 (t, J=6.6 Hz, 2H), 3.46 (t, J=5.7 Hz, 4H), 2.98 (t, J=5.2 Hz, 4H), 2.26-2.09 (m, 4H), 1.57 (s, 4H), 0.35 (s, 4H).

### Example 46: Synthesis of Compound 46

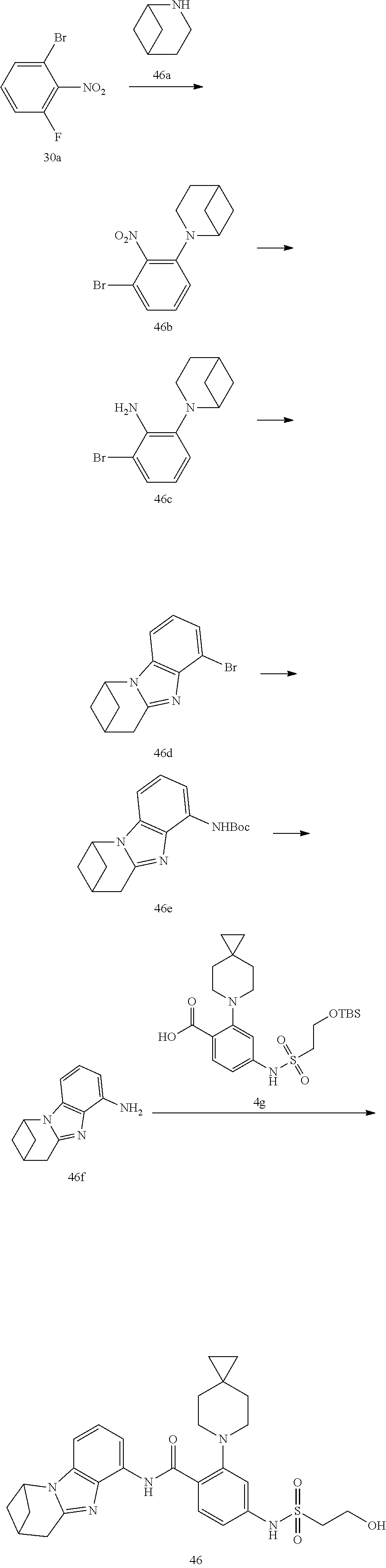

### Step 1: Synthesis of Compound 46b

Compound 46b (640 mg) was obtained through synthesis and purification using a method identical to that described in step 1 of Example 30, with compound 30a (500 mg, 2.27 mmol) and 46a (hydrochloride, 304 mg, 2.27 mmol) as starting materials.

### Step 2: Synthesis of Compound 46c

Compound 46c (490 mg) was obtained through synthesis and purification using a method identical to that described in step 3 of Example 4, with compound 46b (540 mg, 2.15 mmol) as starting material.

### Step 3: Synthesis of Compound 46d

Compound 46d (230 mg) was obtained through synthesis and purification using a method identical to that described in step 3 of Example 30, with compound 46c (470 mg, 1.76 mmol) as starting material.

### Step 4: Synthesis of Compound 46e

Compound 46e (80 mg) was obtained through synthesis and purification using a method identical to that described in step 4 of Example 30, with compound 46d (220 mg, 0.84 mmol) and tert-butyl carbamate (118 mg, 1.0 mmol) as starting materials.

### Step 5: Synthesis of Compound 46f

Compound 46f (45 mg) was obtained through synthesis and purification using a method identical to that described in step 5 of Example 30, with compound 46e (75 mg, 0.25 mmol) as starting material.

MS m/z (ESI): 200.2 [M+H]⁺.

### Step 6: Synthesis of Compound 46

Compound 46 (4.5 mg) was obtained through synthesis and purification using a method identical to that described in step 5 of Example 23, with compound 46f (40 mg, 0.2 mmol) and compound 4g (94 mg, 0.2 mmol) as starting materials.

MS m/z (ESI): 536.2 [M+H]⁺.

¹H NMR (400 MHz, DMSO-d₆) δ 12.64 (s, 1H), 8.30 (d, J=7.8 Hz, 1H), 7.92 (d, J=8.6 Hz, 1H), 7.20 (d, J=8.0 Hz, 1H), 7.11 (t, J=8.0 Hz, 1H), 7.06 (d, J=2.2 Hz, 1H), 6.87 (dd, J=8.7, 2.1 Hz, 1H), 4.96 (q, J=4.7 Hz, 1H), 3.73 (t, J=6.6 Hz, 2H), 3.14 (t, J=6.6 Hz, 2H), 3.03-2.93 (m, 4H), 2.75-2.71 (m, 2H), 2.11-1.76 (m, 5H), 1.68-1.62 (m, 2H), 1.49-1.42 (m, 1H), 0.32 (s, 4H).

### Example 47: Synthesis of Compound 47

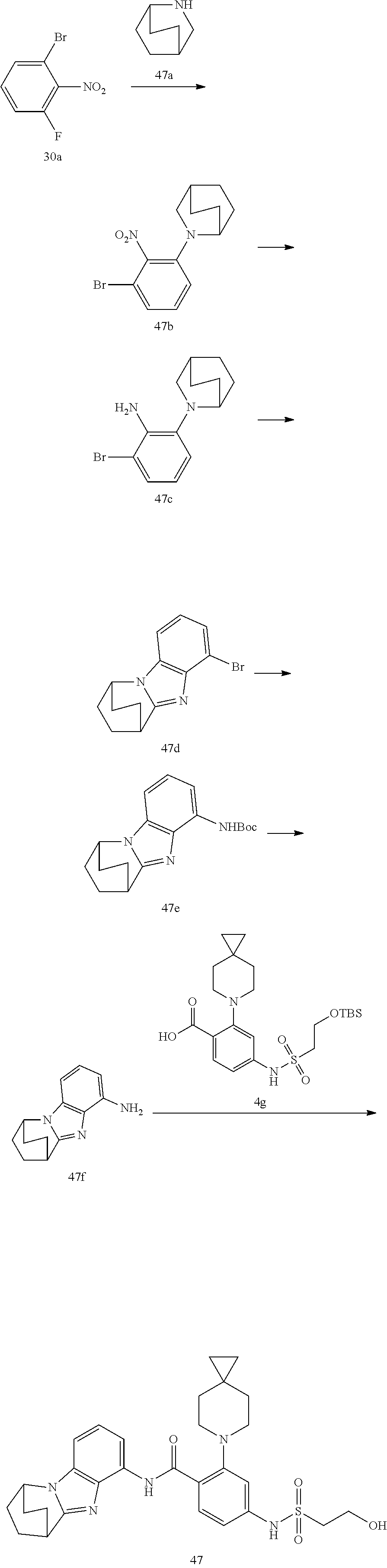

### Step 1: Synthesis of Compound 47b

Compound 47b (1.8 g) was obtained through synthesis and purification using a method identical to that described in step 1 of Example 30, with compound 30a (1.34 g, 6.1 mmol) and 47a (hydrochloride, 857 mg, 5.8 mmol) as starting materials.

### Step 2: Synthesis of Compound 47c

Compound 47c (1.54 g) was obtained through synthesis and purification using a method identical to that described in step 3 of Example 4, with compound 47b (1.8 g, 5.78 mmol) as starting material.

### Step 3: Synthesis of Compound 47d

Compound 47d (500 mg) was obtained through synthesis and purification using a method identical to that described in step 3 of Example 30, with compound 47c (1.53 g, 5.44 mmol) as starting material.

### Step 4: Synthesis of Compound 47e

Compound 47e (500 mg) was obtained through synthesis and purification using a method identical to that described in step 4 of Example 30, with compound 47d (500 mg, 1.80 mmol) as starting material.

### Step 5: Synthesis of Compound 47f

Compound 47f (100 mg) was obtained through synthesis and purification using a method identical to that described in step 5 of Example 30, with compound 47e (500 mg, 1.32 mmol) as starting material.

MS m/z (ESI): 214.2 [M+H]⁺.

### Step 6: Synthesis of Compound 47

Compound 47 (31 mg) was obtained through synthesis and purification using a method identical to that described in step 5 of Example 23, with compound 47f (89 mg, 0.4 mmol) and compound 4g (188 mg, 0.4 mmol) as starting materials.

MS m/z (ESI): 550.1 [M+H]⁺.

¹H NMR (400 MHz, DMSO-d₆) δ 12.45 (s, 1H), 8.30 (dd, J=8.0, 0.9 Hz, 1H), 8.03 (d, J=8.6 Hz, 1H), 7.31 (dd, J=8.1, 1.0 Hz, 1H), 7.26 (d, J=2.2 Hz, 1H), 7.14 (t, J=8.0 Hz, 1H), 7.08 (dd, J=8.6, 2.1 Hz, 1H), 5.02 (s, 1H), 3.77 (t, J=6.5 Hz, 2H), 3.38-3.36 (m, 2H), 3.04-2.91 (m, 4H), 2.18-1.68 (m, 8H), 1.68-1.55 (m, 4H), 0.28 (s, 4H).

### Example 48: Synthesis of Compound 48

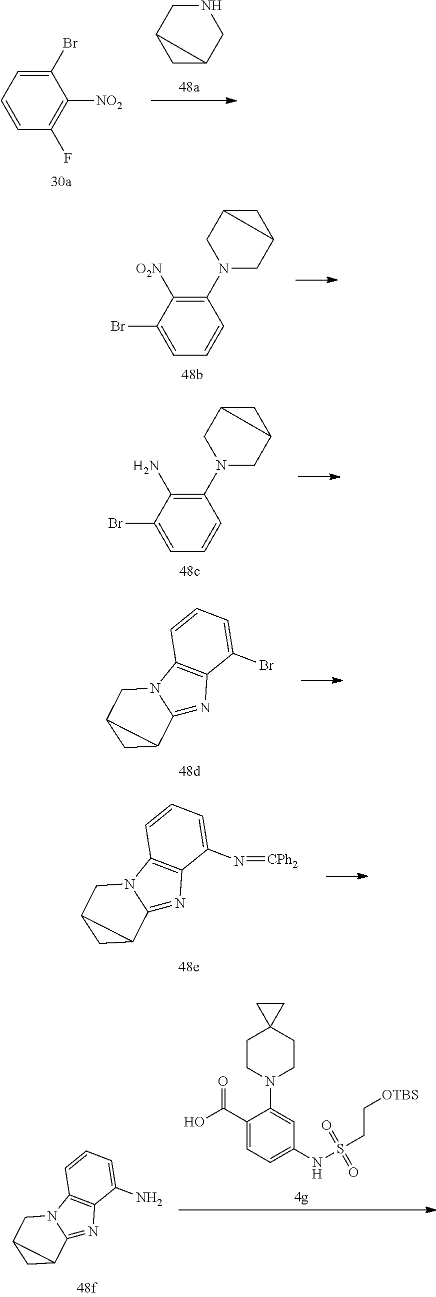

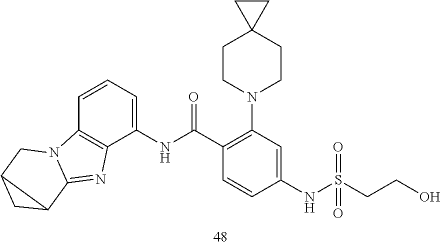 [SMILES: O=C(Nc1cccc2c1nc1n2CC2CC12)c1ccc(NS(=O)(=O)CCO)cc1N1CCC2(CC1)CC2]

### Step 1: Synthesis of Compound 48b

Compound 48b (3.5 g) was obtained through synthesis and purification using a method identical to that described in step 1 of Example 30, with compound 30a (3.00 g, 13.6 mmol) and 48a (hydrochloride, 1.96 g, 16.4 mmol) as starting materials.

### Step 2: Synthesis of Compound 48c

Compound 48c (2.00 g) was obtained through synthesis and purification using a method identical to that described in step 3 of Example 4, with compound 48b (2.26 g, 8.0 mmol) as starting material.

### Step 3: Synthesis of Compound 48d

Compound 48d (850 mg) was obtained through synthesis and purification using a method identical to that described in step 3 of Example 30, with compound 48c (2.00 g, 7.90 mmol) as starting material.

### Step 4: Synthesis of Compound 48e

Compound 48e (300 mg) was obtained through synthesis and purification using a method identical to that described in step 4 of Example 30, with compound 48d (300 mg, 1.20 mmol) as starting material.

### Step 5: Synthesis of Compound 48f

Compound 48f (222 mg) was obtained through synthesis and purification using a method identical to that described in step 5 of Example 30, with compound 48e (300 mg, 0.86 mmol) as starting material.

MS m/z (ESI): 186.1 [M+H]⁺.

### Step 6: Synthesis of Compound 48

Compound 48 (26 mg) was obtained through synthesis and purification using a method identical to that described in step 5 of Example 23, with compound 48f (118 mg, 0.64 mmol) and compound 4g (200 mg, 0.43 mmol) as starting materials.

MS m/z (ESI): 522.1 [M+H]⁺.

¹H NMR (400 MHz, DMSO-d₆) δ 12.30 (s, 1H), 10.16 (s, 1H), 8.26 (d, J=7.7 Hz, 1H), 8.00 (d, J=8.5 Hz, 1H), 7.26 (d, J=2.1 Hz, 1H), 7.15-7.04 (m, 3H), 4.95 (s, 1H), 4.25 (dd, J=11.2, 5.7 Hz, 1H), 4.16 (d, J=11.2 Hz, 1H), 3.76 (t, J=6.5 Hz, 2H), 3.38-3.36 (m, 2H), 3.06-2.93 (m, 4H), 2.64-2.57 (m, 1H), 2.47-2.43 (m, 1H), 1.79 (s, 4H), 1.46-1.38 (m, 1H), 0.80-0.74 (m, 1H), 0.31 (s, 4H).

### Example 49: Synthesis of Compound 49

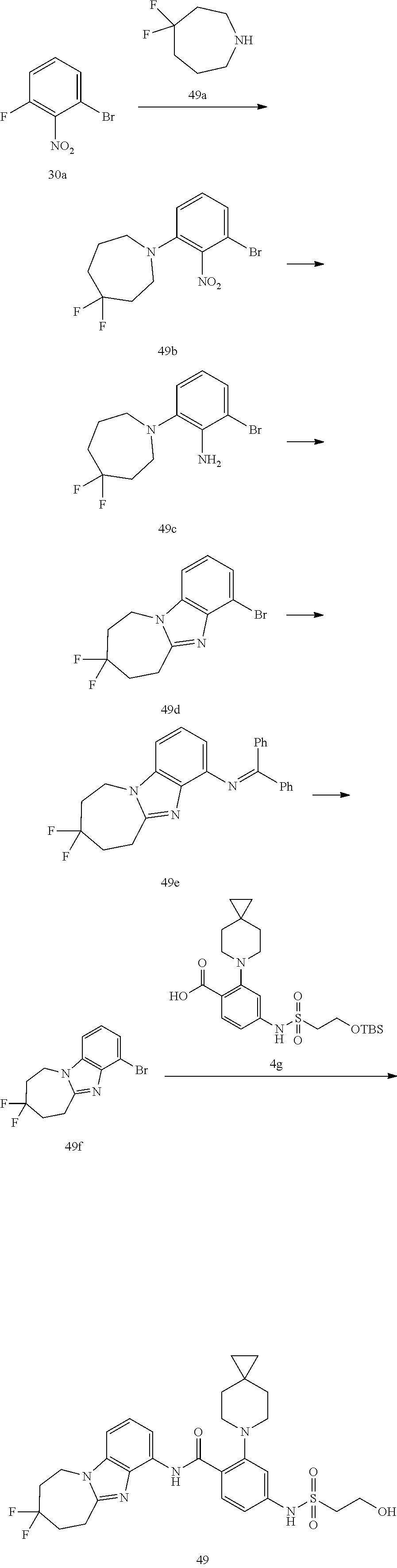

### Step 1: Synthesis of Compound 49b

Compound 49b (1.1 g) was obtained through synthesis and purification using a method identical to that described in step 1 of Example 30, with compound 30a (1.0 g, 4.5 mmol) and 49a (hydrochloride, 0.86 g, 5.0 mmol) as starting materials.

### Step 2: Synthesis of Compound 49c

Compound 49c (0.9 g) was obtained through synthesis and purification using a method identical to that described in step 3 of Example 4, with compound 49b (1.1 g, 3.3 mmol) as starting material.

### Step 3: Synthesis of Compound 49d

Compound 49d (270 mg) was obtained through synthesis and purification using a method identical to that described in step 3 of Example 30, with compound 49c (0.9 g, 3.0 mmol) as starting material.

### Step 4: Synthesis of Compound 49e

Compound 49e (280 mg) was obtained through synthesis and purification using a method identical to that described in step 4 of Example 30, with compound 49d (270 mg, 0.90 mmol) as starting material.

### Step 5: Synthesis of Compound 49f

Compound 49f (121 mg) was obtained through synthesis and purification using a method identical to that described in step 5 of Example 30, with compound 49e (280 mg, 0.70 mmol) as starting material.

MS m/z (ESI): 238.1 [M+H]⁺.

### Step 6: Synthesis of Compound 49

Compound 49 (16 mg) was obtained through synthesis and purification using a method identical to that described in step 5 of Example 23, with compound 49f (80 mg, 0.34 mmol) and compound 4g (158 mg, 0.34 mmol) as starting materials.

MS m/z (ESI): 574.2 [M+H]⁺.

### Example 50: Synthesis of Compound 50

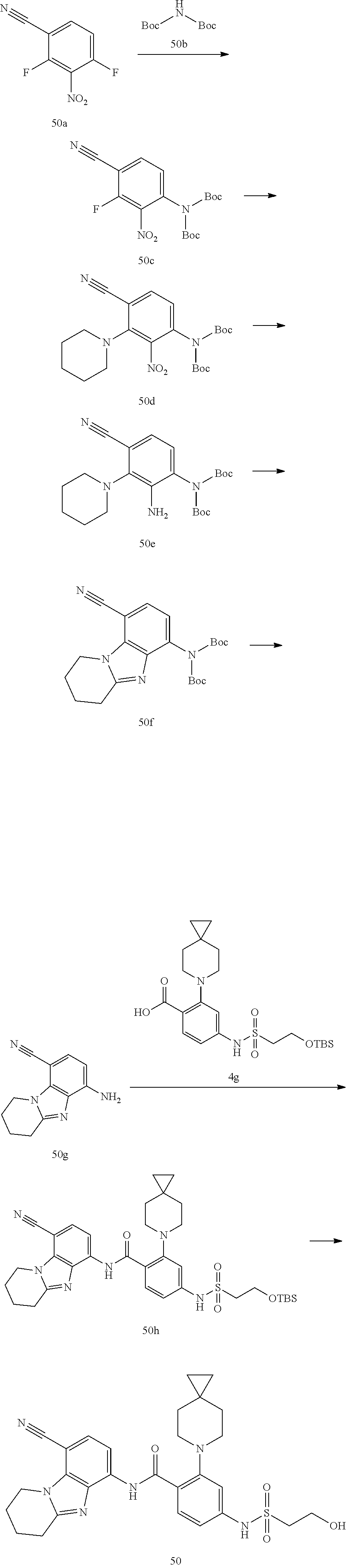

### Step 1: Synthesis of Compound 50c

Compound 50a (200 mg, 1.08 mmol) and compound 50b (235 mg, 1.08 mmol) were dissolved in DMF (4 mL), followed by the addition of sodium hydride (86.4 mg, 2.16 mmol, 60%). The mixture was reacted sufficiently at room temperature. Water (20 mL) was added to quench the reaction, and ethyl acetate (10 mL×3) was added for extraction. The combined organic phases were washed with saturated brine, dried over anhydrous magnesium sulfate, filtered, and concentrated to dryness by rotary evaporation, and the residue was purified by flash column chromatography (petroleum ether:ethyl acetate=5:1) to give compound 50c (244 mg).

### Step 2: Synthesis of Compound 50d

Compound 50d (121 mg) was obtained through synthesis and purification using a method identical to that described in step 1 of Example 30, with compound 50c (122 mg, 0.32 mmol) and piperidine (43 mg, 0.5 mmol) as starting materials.

### Step 3: Synthesis of Compound 50e

Compound 50e (87 mg) was obtained through synthesis and purification using a method identical to that described in step 3 of Example 4, with compound 50d (120 mg, 0.27 mmol) as starting material.

### Step 4: Synthesis of Compound 50f

Compound 50f (62 mg) was obtained through synthesis and purification using a method identical to that described in step 3 of Example 30, with compound 50e (83 mg, 0.21 mmol) as starting material.

### Step 5: Synthesis of Compound 50g

Compound 50f (62 mg, 0.15 mmol) was added to anhydrous dioxane (1 mL), followed by the addition of a solution of hydrochloric acid in dioxane (4 N, 0.2 mL). The mixture was reacted sufficiently at room temperature. The reaction mixture was filtered, and the filter cake was dried to give compound 50g (30 mg, crude product).

MS m/z (ESI): 213.1 [M+H]⁺.

### Step 6: Synthesis of Compound 50h

Compound 50h (13.9 mg) was obtained through synthesis and purification using a method identical to that described in step 3 of Example 5, with compound 4g (70 mg, 0.15 mmol) and 50g (21 mg, 0.1 mmol) as starting materials.

### Step 7: Synthesis of Compound 50

Compound 50 (5.6 mg) was obtained through synthesis and purification using a method identical to that described in step 8 of Example 4, with compound 50h (13.9 mg, 21 μmol) as starting material.

MS m/z (ESI): 549.2 [M+H]⁺.

¹H NMR (400 MHz, DMSO-d₆) δ 12.88 (s, 1H), 8.46 (d, J=8.5 Hz, 1H), 8.03 (d, J=8.6 Hz, 1H), 7.65 (d, J=8.5 Hz, 1H), 7.26 (s, 1H), 7.06 (d, J=8.7 Hz, 1H), 4.45 (t, J=6.1 Hz, 2H), 3.75 (t, J=6.5 Hz, 2H), 3.30 (t, J=6.5 Hz, 2H), 3.05 (t, J=6.3 Hz, 2H), 2.98 (t, J=5.4 Hz, 4H), 2.20-2.06 (m, 2H), 1.97 (t, J=5.7 Hz, 2H), 1.84 (s, 4H), 0.33 (s, 4H).

### Example 51: Synthesis of Compound 51

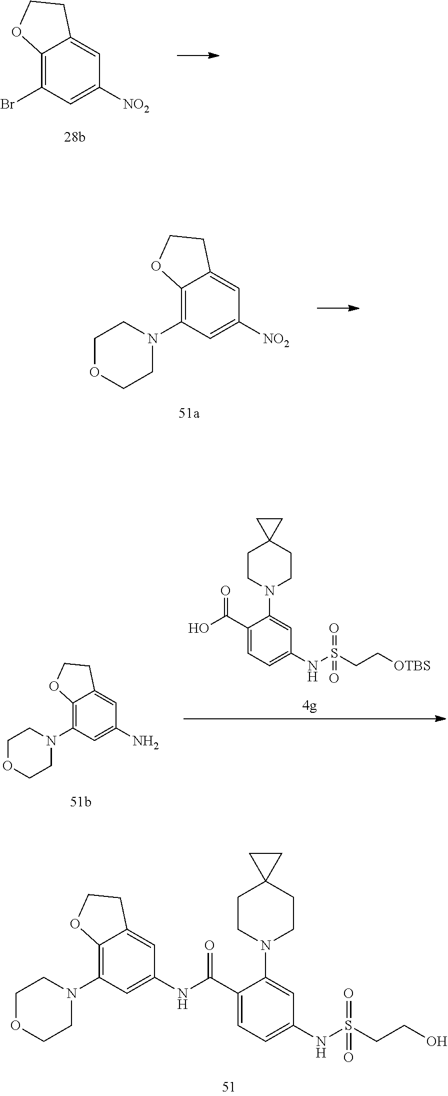 [SMILES: O=[N+]([O-])c1cc(Br)c2c(c1)CCO2 ; O=[N+]([O-])c1cc2c(c(N3CCOCC3)c1)OCC2 ; Nc1cc2c(c(N3CCOCC3)c1)OCC2 ; CC(C)(C)[Si](C)(C)OCCS(=O)(=O)Nc1ccc(C(=O)O)c(N2CCC3(CC2)CC3)c1 ; O=C(Nc1cc2c(c(N3CCOCC3)c1)OCC2)c1ccc(NS(=O)(=O)CCO)cc1N1CCC2(CC1)CC2]

### Step 1: Synthesis of Compound 51a

Compound 51a (80 mg) was obtained through synthesis and purification using a method identical to that described in step 4 of Example 22, with compound 28b (100 mg, 0.41 mmol) and morpholine (36 mg, 0.41 mmol) as starting materials.

### Step 2: Synthesis of Compound 51b

Compound 51b (41 mg) was obtained through synthesis and purification using a method identical to that described in step 3 of Example 4, with compound 51a (60 mg, 0.24 mmol) as starting material.

MS m/z (ESI): 221.1 [M+H]⁺.

### Step 3: Synthesis of Compound 51

Compound 51 (13 mg) was obtained through synthesis and purification using a method identical to that described in step 5 of Example 23, with compound 51b (11 mg, 0.05 mmol) and compound 4g (28 mg, 0.06 mmol) as starting materials.

MS m/z (ESI): 557.2 [M+H]⁺.

¹H NMR (400 MHz, DMSO-d₆) δ 11.60 (s, 1H), 7.83 (d, J=8.4 Hz, 1H), 7.39 (s, 1H), 7.16 (d, J=2.0 Hz, 1H), 7.04-7.00 (m, 2H), 4.52 (t, J=8.8 Hz, 2H), 3.77-3.72 (m, 6H), 3.16 (t, J=8.4 Hz, 2H), 3.14 (d, J=10.0 Hz, 2H), 3.06-3.03 (m, 4H), 2.98-2.95 (m, 4H), 1.95-1.52 (m, 4H), 0.36 (s, 4H).

### Example 52: Synthesis of Compound 52

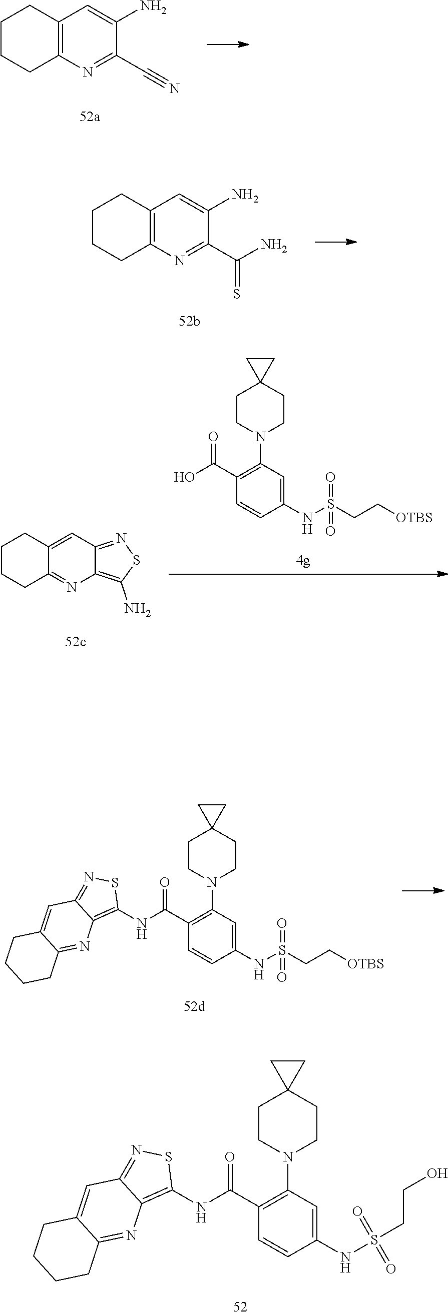 [SMILES: N#Cc1nc2c(cc1N)CCCC2 ; NC(=S)c1nc2c(cc1N)CCCC2 ; CC(C)(C)[Si](C)(C)OCCS(=O)(=O)Nc1ccc(C(=O)O)c(N2CCC3(CC2)CC3)c1 ; Nc1snc2cc3c(nc12)CCCC3 ; CC(C)(C)[Si](C)(C)OCCS(=O)(=O)Nc1ccc(C(=O)Nc2snc3cc4c(nc23)CCCC4)c(N2CCC3(CC2)CC3)c1 ; O=C(Nc1snc2cc3c(nc12)CCCC3)c1ccc(NS(=O)(=O)CCO)cc1N1CCC2(CC1)CC2]

### Step 1: Synthesis of Compound 52b

Compound 52b (45 mg) was obtained through synthesis and purification using a method identical to that described in step 1 of Example 32, with compound 52a (45 mg, 0.26 mmol) as starting material.

### Step 2: Synthesis of Compound 52c

Compound 52c (42 mg) was obtained through synthesis and purification using a method identical to that described in step 2 of Example 32, with compound 52b (45 mg, 0.22 mmol) as starting material.

MS m/z (ESI): 206.1 [M+H]⁺.

### Step 3: Synthesis of Compound 52d

Compound 52d (100 mg) was obtained through synthesis and purification using a method identical to that described in step 3 of Example 5, with compound 4g (93 mg, 0.2 mmol) and 52c (41 mg, 0.2 mmol) as starting materials.

### Step 4: Synthesis of Compound 52

Compound 52 (8.0 mg) was obtained through synthesis and purification using a method identical to that described in step 8 of Example 4, with compound 52d (100 mg, 15 μmol) as starting material.

MS m/z (ESI): 542.2 [M+H]⁺.

¹H NMR (400 MHz, DMSO-d₆) δ 14.92 (s, 1H), 10.37 (s, 1H), 8.19 (d, J=8.7 Hz, 1H), 7.73 (s, 1H), 7.42 (d, J=2.1 Hz, 1H), 7.23 (dd, J=8.7, 2.1 Hz, 1H), 4.97 (t, J=5.2 Hz, 1H), 3.78 (q, J=6.0 Hz, 2H), 3.40 (t, J=6.4 Hz, 2H), 3.09 (t, J=6.6 Hz, 2H), 3.04 (s, 4H), 2.94 (t, J=6.3 Hz, 2H), 2.28-1.38 (m, 8H), 0.43 (s, 4H).

### Example 53 and Example 54: Synthesis of Compound 53 and Compound 54

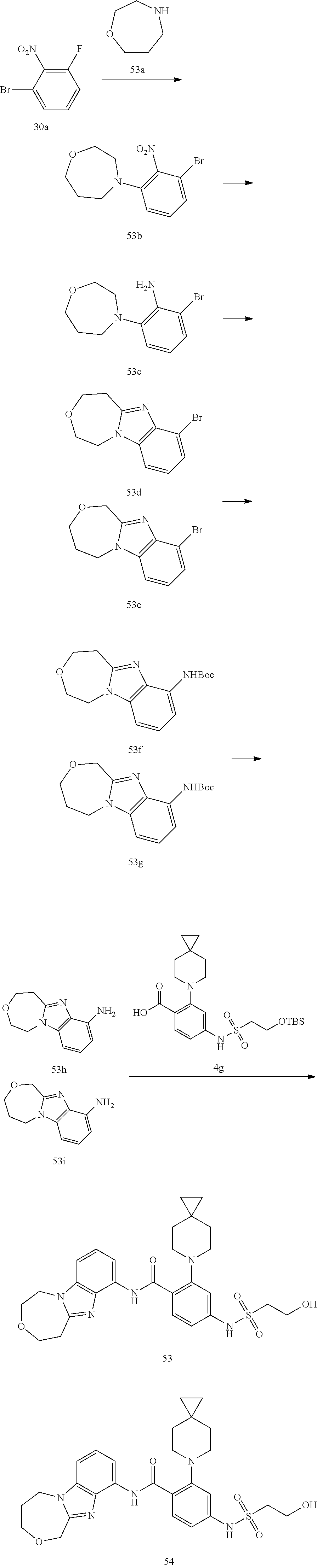

### Step 1: Synthesis of Compound 53b

Compound 53b (5.1 g) was obtained through synthesis and purification using a method identical to that described in step 1 of Example 30, with compound 30a (4.0 g, 18.2 mmol) and 53a (hydrochloride, 2.5 g, 18.2 mmol) as starting materials.

### Step 2: Synthesis of Compound 53c

Compound 53c (3.1 g) was obtained through synthesis and purification using a method identical to that described in step 3 of Example 4, with compound 53b (5.1 g, 17 mmol) as starting material.

Step 3: Synthesis of Compound 53d and compound 53e

The mixture (1.2 g) of compound 53d and compound 53e was obtained through synthesis and purification using a method identical to that described in step 3 of Example 30, with compound 53c (3.0 g, 11 mmol) as starting material.

### Step 4: Synthesis of Compound 53f and Compound 53g

The mixture (0.24 g) of compound 53f and compound 53g was obtained through synthesis and purification using a method identical to that described in step 4 of Example 30, with the mixture (276 mg, 1.0 mmol) of compound 53d and compound 53e and tert-butyl carbamate (140 mg, 1.2 mmol) as starting materials.

Step 5: synthesis of Compound 53 h and Compound 53i 109501 The mixture (110 mg) of compound 53 h and compound 53i was obtained through synthesis and purification using a method identical to that described in step 5 of Example 30, with the mixture (240 mg, 0.79 mmol) of compound 53f and compound 53g as starting material.

MS m/z (ESI): 204.1 [M+H]⁺.

### Step 6: Synthesis of Compound 53 and Compound 54

The final products were obtained through synthesis and purification using a method identical to that described in step of Example 23, with the mixture (110 mg, 0.54 mmol) of compound 53 h and compound 53i and compound 4g (281 mg, 0.6 mmol) as starting materials.

### Compound 53 (25 mg)

MS m/z (ESI): 540.2 [M+H]⁺.

¹H NMR (400 MHz, DMSO-d₆) δ 12.53 (s, 1H), 8.34 (dd, J=7.7, 1.1 Hz, 1H), 8.01 (d, J=8.6 Hz, 1H), 7.25-7.14 (m, 3H), 7.03 (dd, J=8.6, 2.1 Hz, 1H), 4.45-4.33 (m, 2H), 3.92-3.83 (m, 4H), 3.75 (t, J=6.5 Hz, 2H), 3.31-3.26 (m, 4H), 3.03-2.92 (m, 4H), 2.07-1.54 (m, 4H), 0.30 (s, 4H).

### Compound 54 (34 mg)

MS m/z (ESI): 540.2 [M+H]⁺

¹H NMR (400 MHz, DMSO-d₆) δ 12.59 (s, 1H), 8.36 (d, J=7.8 Hz, 1H), 8.04 (d, J=8.6 Hz, 1H), 7.30 (d, J=8.0 Hz, 1H), 7.27-7.19 (m, 2H), 7.05 (dd, J=8.6, 2.1 Hz, 1H), 4.89 (s, 2H), 4.47-4.38 (m, 2H), 4.11-4.03 (m, 2H), 3.75 (t, J=6.5 Hz, 2H), 3.30 (t, J=6.5 Hz, 2H), 2.98 (t, J=5.3 Hz, 4H), 2.13-1.50 (m, 6H), 0.32 (s, 4H).

### Example 55 and Example 56: Synthesis of Compound 55 and Compound 56

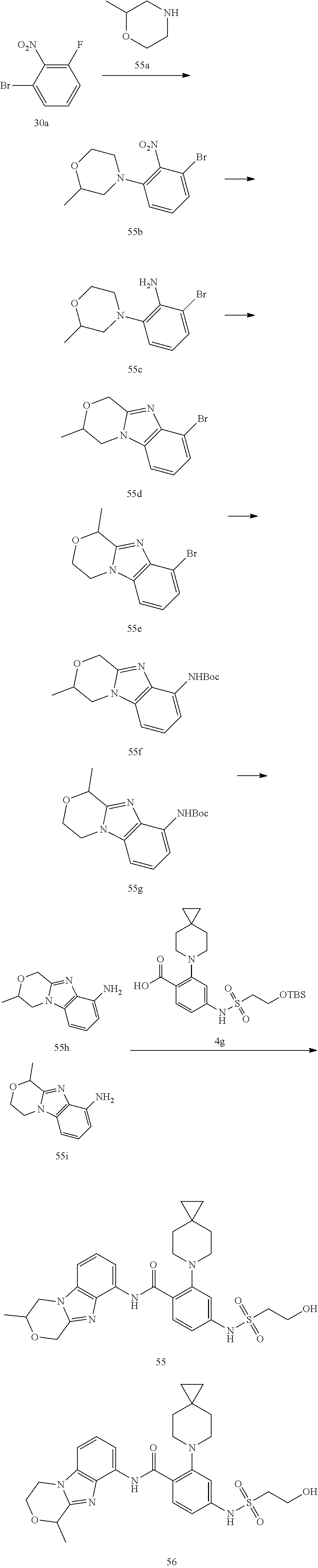

### Step 1: Synthesis of Compound 55b

Compound 55b (2.8 g) was obtained through synthesis and purification using a method identical to that described in step 1 of Example 30, with compound 30a (2.4 g, 10.9 mmol) and 55a (1.32 g, 13 mmol) as starting materials.

### Step 2: Synthesis of Compound 55c

Compound 55c (1.1 g) was obtained through synthesis and purification using a method identical to that described in step 3 of Example 4, with compound 55b (2.8 g, 9.3 mmol) as starting material.

Step 3: Synthesis of Compound 55d and compound 55e

The mixture (0.3 g) of compound 55d and compound 55e was obtained through synthesis and purification using a method identical to that described in step 3 of Example 30, with compound 55c (1.1 g, 4.1 mmol) as starting material.

### Step 4: Synthesis of Compound 55f and Compound 55g

The mixture (0.13 g) of compound 55f and compound 55g was obtained through synthesis and purification using a method identical to that described in step 4 of Example 30, with the mixture (150 mg, 0.56 mmol) of compound 55d and compound 55e and tert-butyl carbamate (132 mg, 1.1 mmol) as starting materials.

### Step 5: Synthesis of Compound 55 h and Compound 55i

The mixture (50 mg) of compound 55 h and compound 55i was obtained through synthesis and purification using a method identical to that described in step 5 of Example 30, with the mixture (130 mg, 0.43 mmol) of compound 55f and compound 55g as starting material.

MS m/z (ESI): 204.1 [M+H]⁺.

### Step 6: Synthesis of Compound 55 and Compound 56

The final products were obtained through synthesis and purification using a method identical to that described in step 5 of Example 23, with the mixture (47 mg, 0.23 mmol) of compound 55 h and compound 55i and compound 4g (122 mg, 0.26 mmol) as starting materials.

Compound 55 (25 mg)

MS m/z (ESI): 540.1 [M+H]⁺.

¹H NMR (400 MHz, DMSO-d₆) δ 12.50 (s, 1H), 8.38 (dd, J=6.5, 2.4 Hz, 1H), 8.04 (d, J=8.6 Hz, 1H), 7.27-7.18 (m, 3H), 7.08 (dd, J=8.6, 2.1 Hz, 1H), 5.02 (d, J=15.8 Hz, 1H), 4.97 (d, J=15.7 Hz, 1H), 4.36 (dd, J=12.0, 3.3 Hz, 1H), 4.25-4.16 (m, 1H), 3.84 (dd, J=12.0, 10.4 Hz, 1H), 3.76 (t, J=6.5 Hz, 2H), 3.37-3.35 (m, 2H), 3.03-2.92 (m, 4H), 1.95-1.54 (m, 4H), 1.39 (d, J=6.1 Hz, 3H), 0.31 (s, 4H).

Compound 56 (20 mg)

MS m/z (ESI): 540.1 [M+H]⁺.

¹H NMR (400 MHz, DMSO-d₆) δ 12.15 (s, 1H), 8.45 (dd, J=6.2, 2.7 Hz, 1H), 8.00 (d, J=8.6 Hz, 1H), 7.26-7.19 (m, 3H), 7.05 (dd, J=8.6, 2.0 Hz, 1H), 5.03 (q, J=6.6 Hz, 1H), 4.39-4.31 (m, 1H), 4.28-4.14 (m, 2H), 4.10-4.01 (m, 1H), 3.76 (t, J=6.5 Hz, 2H), 3.32-3.30 (m, 2H), 3.06-2.93 (m, 4H), 1.96-1.56 (m, 7H), 0.38-0.23 (m, 4H).

### Example 57: Synthesis of Compound 57

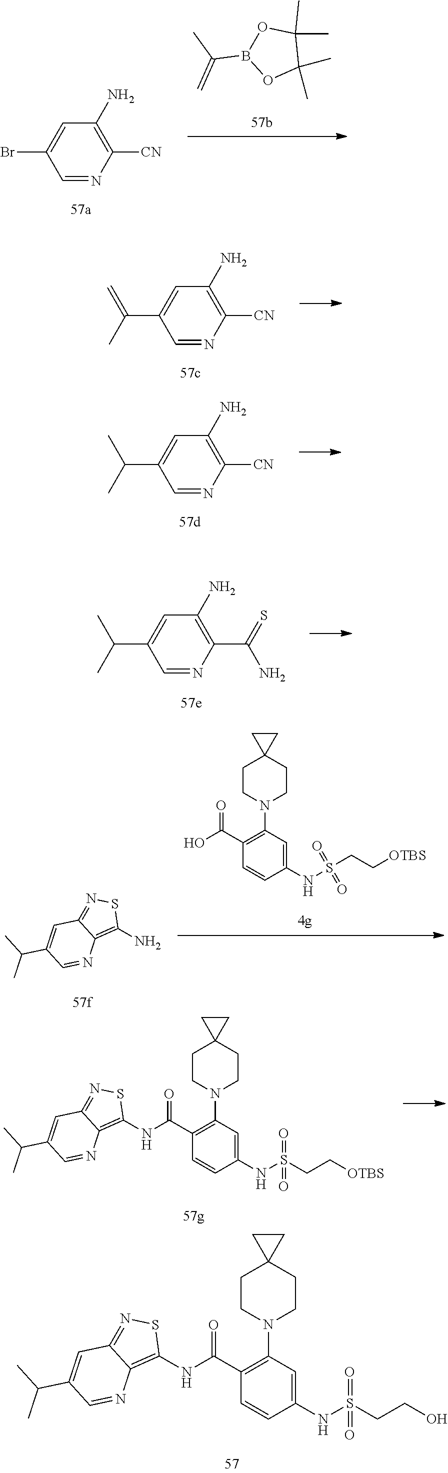

### Step 1: Synthesis of Compound 57c

Compound 57c (1.3 g) was obtained through synthesis and purification using a method identical to that described in step 2 of Example 27, with compound 57a (2.0 g, 10.1 mmol) and compound 57b (3.4 g, 20.2 mmol) as starting materials.

### Step 2: Synthesis of Compound 57d

Palladium on carbon (130 mg) was added to a solution of compound 57c (1.3 g, 9.30 mmol) in methanol (20 mL), and the mixture was reacted sufficiently at room temperature in a hydrogen atmosphere. The reaction mixture was filtered and concentrated to dryness by rotary evaporation, and the residue was purified by column chromatography (petroleum ether:ethyl acetate=100:0-70:30) to give compound 57d (1.0 g).

### Step 3: Synthesis of Compound 57e

Compound 57e (0.92 g) was obtained through synthesis and purification using a method identical to that described in step 1 of Example 32, with compound 57d (1.0 mg, 6.2 mmol) as starting material.

### Step 4: Synthesis of Compound 57f

Compound 57f (0.60 g) was obtained through synthesis and purification using a method identical to that described in step 2 of Example 32, with compound 57e (0.90 g, 4.6 mmol) as starting material.

MS m/z (ESI): 194.1 [M+H]⁺.

### Step 5: Synthesis of Compound 57g

Compound 57g (55 mg) was obtained through synthesis and purification using a method identical to that described in step 3 of Example 5, with compound 4g (70 mg, 0.15 mmol) and 57f (29 mg, 0.15 mmol) as starting materials.

### Step 6: Synthesis of Compound 57

Compound 57 (13 mg) was obtained through synthesis and purification using a method identical to that described in step 8 of Example 4, with compound 57g (50 mg, 78 μmol) as starting material.

MS m/z (ESI): 530.1 [M+H]⁺.

¹H NMR (400 MHz, DMSO-d₆) δ 15.00 (s, 1H), 10.37 (s, 1H), 8.60 (d, J=1.9 Hz, 1H), 8.18 (d, J=8.6 Hz, 1H), 7.84 (d, J=1.5 Hz, 1H), 7.41 (d, J=2.2 Hz, 1H), 7.22 (dd, J=8.7, 2.1 Hz, 1H), 4.96 (s, 1H), 3.83-3.73 (m, 2H), 3.40 (t, J=6.4 Hz, 2H), 3.16 (p, J=6.9 Hz, 1H), 3.04 (t, J=5.4 Hz, 4H), 1.87 (s, 4H), 1.33 (d, J=6.9 Hz, 6H), 0.43 (s, 4H).

### Example 58: Synthesis of Compound 58

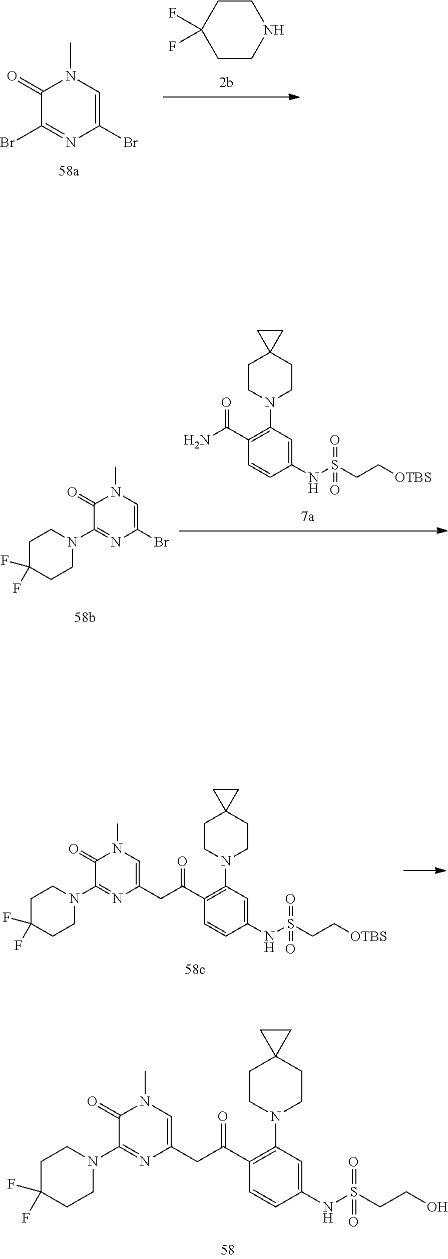

### Step 1: Synthesis of Compound 58b

Compound 58b (194 mg) was obtained through synthesis and purification using a method identical to that described in step 1 of Example 30, with compound 58a (0.2 g, 0.75 mmol) and 2b (91 mg, 0.75 mmol) as starting materials.

### Step 2: Synthesis of Compound 58c

Compound 58c (12 mg) was obtained through synthesis and purification using a method identical to that described in step 2 of Example 7, with compound 7a (31 mg, 0.065 mmol) and 58b (20 mg, 0.065 mmol) as starting materials.

### Step 3: Synthesis of Compound 58

Compound 58 (2.2 mg) was obtained through synthesis and purification using a method identical to that described in step 8 of Example 4, with compound 58c (11 mg, 16 μmol) as starting material.

MS m/z (ESI): 581.4 [M+H]⁺.

¹H NMR (400 MHz, DMSO-d₆) δ 12.70 (s, 1H), 10.15 (s, 1H), 8.03 (d, J=8.6 Hz, 1H), 7.96 (s, 1H), 7.25 (d, J=2.2 Hz, 1H), 7.12 (dd, J=8.6, 2.1 Hz, 1H), 4.94 (s, 1H), 3.99 (s, 4H), 3.75 (t, J=6.5 Hz, 2H), 3.45 (s, 3H), 3.36 (d, J=6.5 Hz, 2H), 2.95 (s, 4H), 2.16-1.99 (m, 4H), 1.72 (m, 4H), 0.39 (s, 4H).

### Example 59: Synthesis of Compound 59

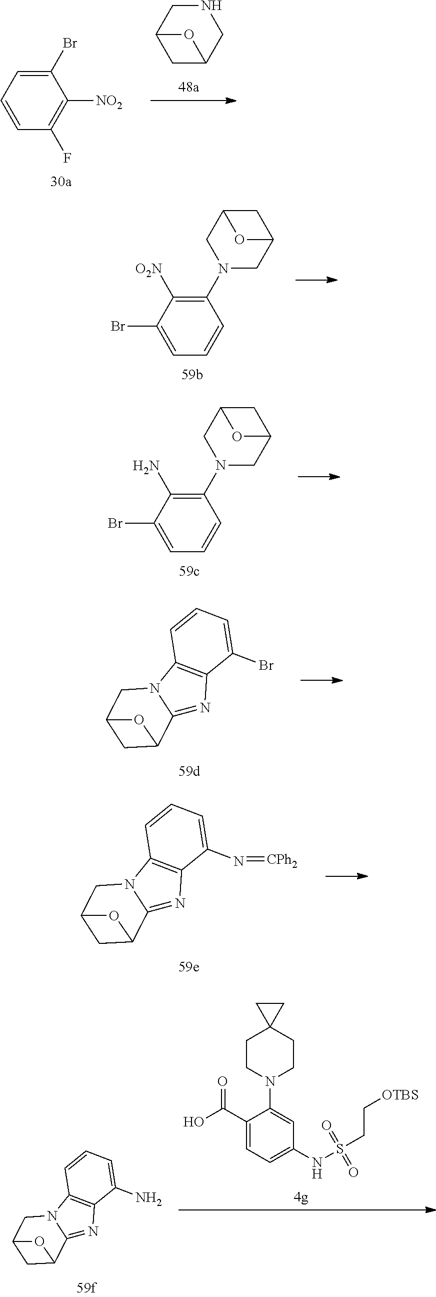

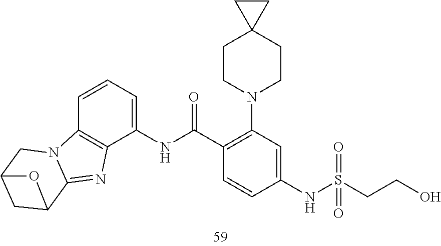 [SMILES: O=C(Nc1cccc2c1nc1n2CC2CC1O2)c1ccc(NS(=O)(=O)CCO)cc1N1CCC2(CC1)CC2]

### Step 1: Synthesis of Compound 59b

Compound 59b (262 mg) was obtained through synthesis and purification using a method identical to that described in step 1 of Example 30, with compound 30a (0.3 g, 1.36 mmol) and 59a (185 mg, 1.36 mmol) as starting materials.

### Step 2: Synthesis of Compound 59c

Compound 59c (188 mg) was obtained through synthesis and purification using a method identical to that described in step 3 of Example 4, with compound 59b (262 mg, 0.88 mmol) as starting material.

### Step 3: Synthesis of Compound 59d

Compound 59d (60 mg) was obtained through synthesis and purification using a method identical to that described in step 3 of Example 30, with compound 59c (188 mg, 0.7 mmol) as starting material.

### Step 4: Synthesis of Compound 59e

Compound 59e (44 mg) was obtained through synthesis and purification using a method identical to that described in step 4 of Example 30, with compound 59d (59 mg, 0.22 mmol) as starting material.

### Step 5: Synthesis of Compound 59f

Compound 59f (18.6 mg) was obtained through synthesis and purification using a method identical to that described in step 5 of Example 30, with compound 59e (44 mg, 0.12 mmol) as starting material.

MS m/z (ESI): 202.1 [M+H]⁺.

### Step 6: Synthesis of Compound 59

Compound 59 (3.6 mg) was obtained through synthesis and purification using a method identical to that described in step 5 of Example 23, with compound 59f (12 mg, 0.06 mmol) and compound 4g (28 mg, 0.06 mmol) as starting materials.

MS m/z (ESI): 538.3 [M+H]⁺.

### Example 60: Synthesis of Compound 60

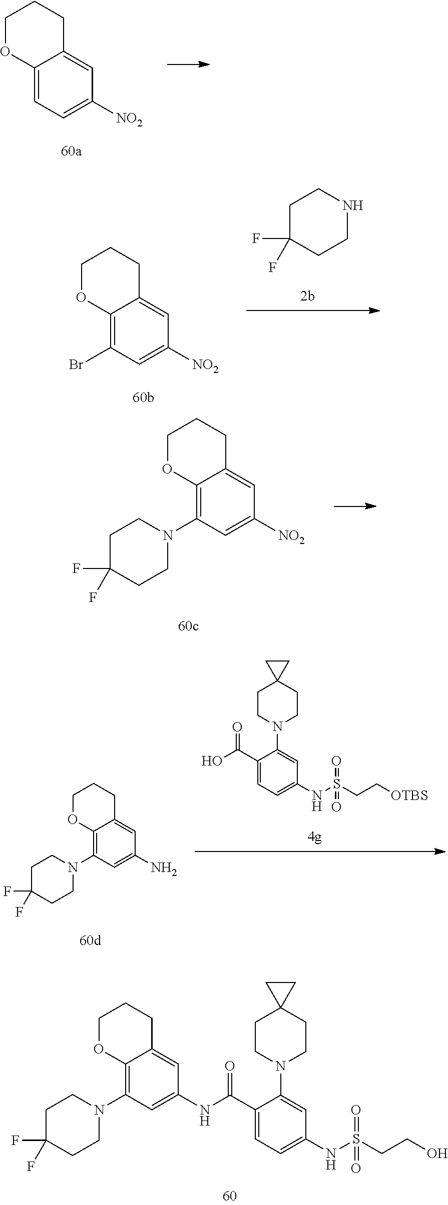

### Step 1: Synthesis of Compound 60b

Compound 60b (110 mg) was obtained through synthesis and purification using a method identical to that described in step 1 of Example 28, with compound 60a (100 mg, 0.55 mmol) as starting material.

### Step 2: Synthesis of Compound 60c

Compound 60c (72 mg) was obtained through synthesis and purification using a method identical to that described in step 4 of Example 22, with compound 60b (110 mg, 0.41 mmol) and 2b (hydrochloride, 60 mg, 0.41 mmol) as starting materials.

### Step 3: Synthesis of Compound 60d

Compound 60d (50 mg) was obtained through synthesis and purification using a method identical to that described in step 3 of Example 4, with compound 60c (70 mg, 0.23 mmol) as starting material.

MS m/z (ESI): 269.1 [M+H]⁺.

### Step 4: Synthesis of Compound 60

Compound 60 (11 mg) was obtained through synthesis and purification using a method identical to that described in step 5 of Example 23, with compound 60d (14 mg, 0.05 mmol) and compound 4g (23 mg, 0.05 mmol) as starting materials.

MS m/z (ESI): 605.3 [M+H]⁺.

### Example 61: Synthesis of Compound 61

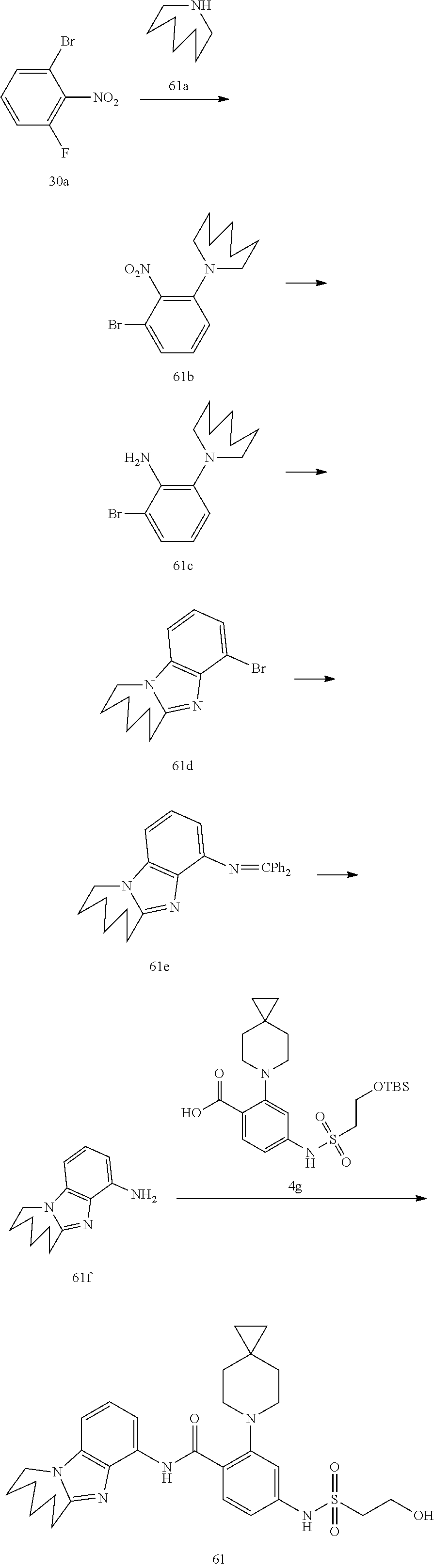

### Step 1: Synthesis of Compound 61b

Compound 61b (0.7 g) was obtained through synthesis and purification using a method identical to that described in step 1 of Example 30, with compound 30a (2.14 g, 9.7 mmol) and 61a (304 mg, 2.3 mmol) as starting materials.

### Step 2: Synthesis of Compound 61c

Compound 61c (520 mg) was obtained through synthesis and purification using a method identical to that described in step 3 of Example 4, with compound 61b (616 mg, 2.0 mmol) as starting material.

### Step 3: Synthesis of Compound 61d

Compound 61d (360 mg) was obtained through synthesis and purification using a method identical to that described in step 3 of Example 30, with compound 61c (520 mg, 1.8 mmol) as starting material.

### Step 4: Synthesis of Compound 61e

Compound 61e (234 mg) was obtained through synthesis and purification using a method identical to that described in step 4 of Example 30, with compound 61d (210 mg, 1.0 mmol) as starting material.

### Step 5: Synthesis of Compound 61f

Compound 61f (116 mg) was obtained through synthesis and purification using a method identical to that described in step 5 of Example 30, with compound 61e (230 mg, 0.6 mmol) as starting material.

MS m/z (ESI): 216.1 [M+H]⁺.

### Step 6: Synthesis of Compound 61

Compound 61 (26 mg) was obtained through synthesis and purification using a method identical to that described in step 5 of Example 23, with compound 61f (22 mg, 0.1 mmol) and compound 4g (47 mg, 0.1 mmol) as starting materials.

MS m/z (ESI): 552.2 [M+H]⁺.

¹H NMR (400 MHz, DMSO-d₆) δ 12.41 (s, 1H), 10.14 (s, 1H), 8.32 (dd, J=7.9, 1.0 Hz, 1H), 8.04 (d, J=8.6 Hz, 1H), 7.28 (d, J=2.2 Hz, 1H), 7.24 (dd, J=8.1, 1.0 Hz, 1H), 7.15 (t, J=8.0 Hz, 1H), 7.09 (dd, J=8.6, 2.1 Hz, 1H), 4.93 (s, 1H), 4.38 (t, J=5.9 Hz, 2H), 3.84-3.68 (m, 2H), 3.36 (t, J=6.5 Hz, 2H), 3.09-3.02 (m, 2H), 3.02-2.96 (m, 4H), 2.05-1.80 (m, 4H), 1.80-1.59 (m, 4H), 1.50-1.40 (m, 2H), 1.17-1.08 (m, 2H), 0.30 (s, 4H).

### Example 62 and Example 63: Synthesis of Compound 62 and Compound 63

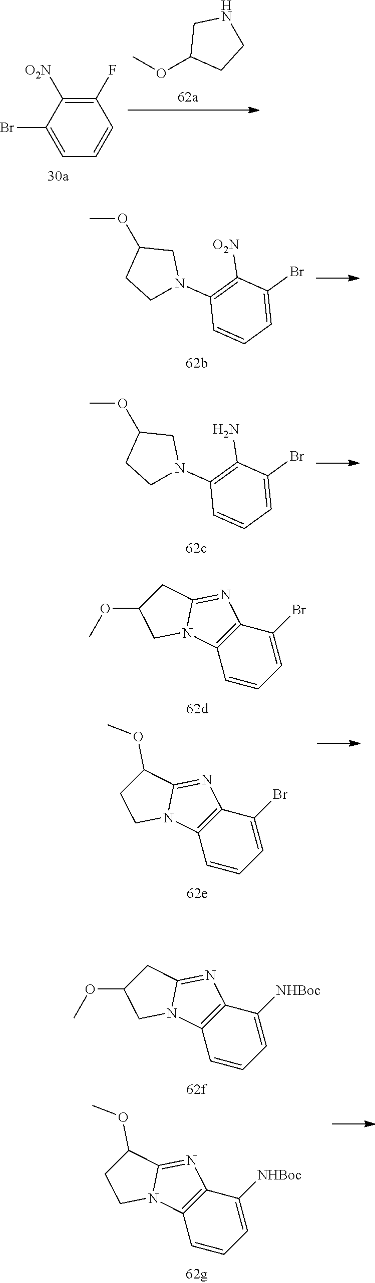

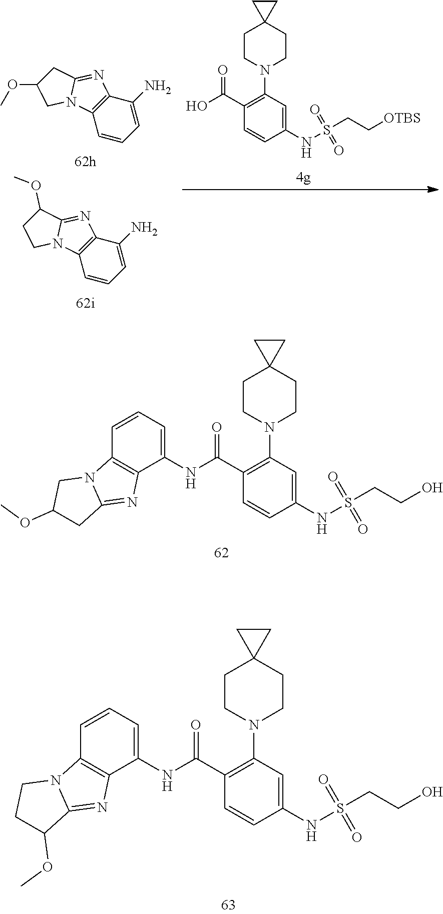 [SMILES: COC1Cc2nc3c(N)cccc3n2C1 ; CC(C)(C)[Si](C)(C)OCCS(=O)(=O)Nc1ccc(C(=O)O)c(N2CCC3(CC2)CC3)c1 ; COC1CCn2c1nc1c(N)cccc12 ; COC1Cc2nc3c(NC(=O)c4ccc(NS(=O)(=O)CCO)cc4N4CCC5(CC4)CC5)cccc3n2C1 ; COC1CCn2c1nc1c(NC(=O)c3ccc(NS(=O)(=O)CCO)cc3N3CCC4(CC3)CC4)cccc12]

### Step 1: Synthesis of Compound 62b

Compound 62b (1.45 g) was obtained through synthesis and purification using a method identical to that described in step 1 of Example 30, with compound 30a (1.08 g, 4.9 mmol) and 62a (0.5 g, 4.9 mmol) as starting materials.

### Step 2: Synthesis of Compound 62c

Compound 62c (1.2 g) was obtained through synthesis and purification using a method identical to that described in step 3 of Example 4, with compound 62b (1.45 g, 4.5 mmol) as starting material.

Step 3: Synthesis of Compound 62d and compound 62e

The mixture (0.81 g) of compound 62d and compound 62e was obtained through synthesis and purification using a method identical to that described in step 3 of Example 30, with compound 62c (1.2 g, 4.4 mmol) as starting material.

### Step 4: Synthesis of Compound 62f and Compound 62g

The mixture (0.65 g) of compound 62f and compound 62g was obtained through synthesis and purification using a method identical to that described in step 4 of Example 30, with the mixture (0.81 g, 3.0 mmol) of compound 62d and compound 62e and tert-butyl carbamate (0.39 g, 3.3 mmol) as starting materials.

### Step 5: Synthesis of Compound 62 h and Compound 62i

The mixture (130 mg) of compound 62 h and compound 62i was obtained through synthesis and purification using a method identical to that described in step 5 of Example 30, with the mixture (0.64 g, 2.1 mmol) of compound 62f and compound 62g as starting material.

MS m/z (ESI): 204.1 [M+H]⁺

### Step 6: Synthesis of Compound 62 and Compound 63

The final products were obtained through synthesis and purification using a method identical to that described in step 5 of Example 23, with the mixture (31 mg, 0.15 mmol) of compound 62 h and compound 62i and compound 4g (93 mg, 0.2 mmol) as starting materials.

Compound 62 (13 mg)

MS m/z (ESI): 540.2 [M+H]⁺.

¹H NMR (400 MHz, DMSO-d₆) δ 12.37 (s, 1H), 8.31 (dd, J=6.9, 2.1 Hz, 1H), 7.99 (d, J=8.5 Hz, 1H), 7.22 (d, J=2.1 Hz, 1H), 7.19-7.11 (m, 2H), 7.04 (dd, J=8.7, 2.0 Hz, 1H), 4.79-4.72 (m, 1H), 4.36 (dd, J=11.5, 5.6 Hz, 1H), 4.17 (dd, J=11.4, 2.2 Hz, 1H), 3.76 (t, J=6.5 Hz, 2H), 3.36 (s, 3H), 3.32-3.28 (m, 3H), 3.07-2.87 (m, 5H), 1.78 (s, 4H), 0.30 (s, 4H).

Compound 63 (4 mg)

MS m/z (ESI): 540.2 [M+H]⁺.

¹H NMR (400 MHz, DMSO-d₆) δ 12.25 (s, 1H), 8.42 (dd, J=6.9, 2.1 Hz, 1H), 7.96 (d, J=8.5 Hz, 1H), 7.27-7.13 (m, 3H), 6.99 (d, J=8.7 Hz, 1H), 4.86-4.77 (m, 1H), 4.27-4.13 (m, 2H), 3.75 (t, J=6.5 Hz, 2H), 3.49 (s, 3H), 3.28-3.23 (m, 2H), 3.07-2.91 (m, 5H), 2.62-2.56 (m, 1H), 1.93-1.59 (m, 4H), 0.29 (d, J=6.4 Hz, 4H).

### Example 64: Synthesis of Compound 64

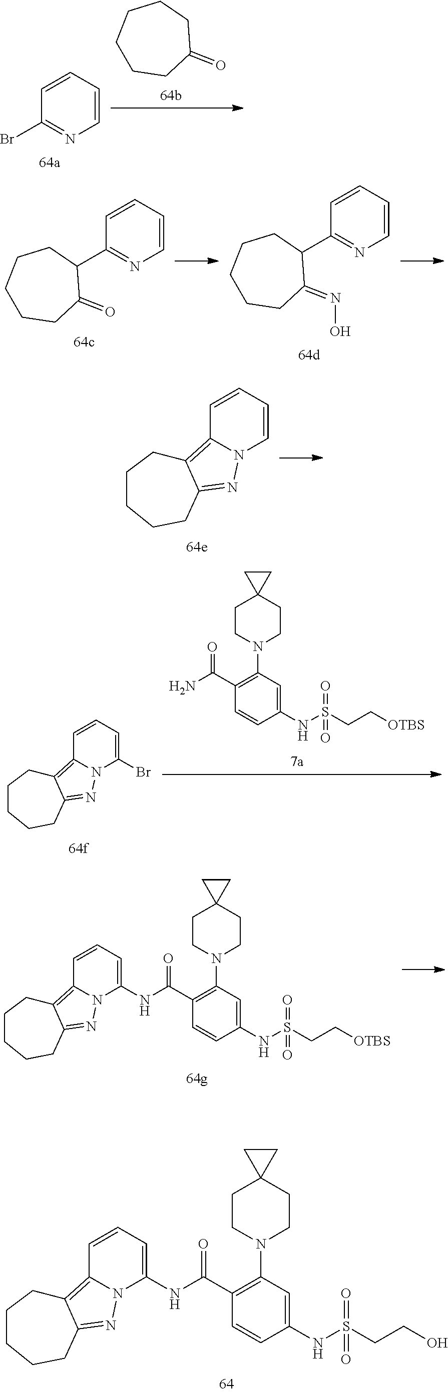

### Step 1: Synthesis of Compound 64c

Compound 64a (300 mg, 1.9 mmol), compound 64b (214 mg, 1.9 mmol), and sodium tert-butoxide (366 mg, 3.8 mmol) were added to THE (10 mL), followed by the addition of XPhos Pd G2 (73 mg, 95.5 mol) in an argon atmosphere. The mixture was reacted sufficiently at room temperature. Water (20 mL) was added to the system, and ethyl acetate (10 mL×3) was added for extraction. The combined organic phases were washed with saturated brine, dried over anhydrous magnesium sulfate, filtered, and concentrated to dryness by rotary evaporation, and the residue was purified by flash column chromatography (petroleum ether:ethyl acetate=5:1) to give compound 64c (180 mg).

### Step 2: Synthesis of Compound 64d

Compound 64c (180 mg, 0.96 mmol), hydroxylamine hydrochloride (66 mg, 0.96 mmol), and sodium acetate (117 mg, 1.43 mmol) were added to anhydrous methanol (10 mL), and the mixture was reacted sufficiently at 70 °C. After the reaction mixture was cooled, a saturated aqueous sodium bicarbonate solution (20 mL) was added to the system, and ethyl acetate (10 mL×3) was added for extraction. The combined organic phases were washed with saturated brine, dried over anhydrous magnesium sulfate, filtered, and concentrated to dryness by rotary evaporation, to give compound 64d (145 mg).

### Step 3: Synthesis of Compound 64e

Compound 64d (145 mg, 0.66 mmol) was added to dichloromethane (0.3 mL), followed by the addition of p-toluenesulfonyl chloride (125 mg, 66 mmol) and triethylamine (199 mg, 1.98 mmol) at 0 °C. The mixture was reacted sufficiently at room temperature. Water (5 mL) was added to the system, and ethyl acetate (5 mL×3) was added for extraction. The combined organic phases were washed with saturated brine, dried over anhydrous magnesium sulfate, filtered, and concentrated to dryness by rotary evaporation, and the residue was purified by flash column chromatography (petroleum ether:ethyl acetate=4:1) to give compound 64e (44 mg).

### Step 4: Synthesis of Compound 64f

Compound 64e (44 mg, 0.22 mmol) was dissolved in anhydrous THE (0.3 mL), followed by the addition of n-butyllithium (0.2 mL, 0.32 mmol, 1.6 M) at −60 °C. The mixture was reacted sufficiently at this temperature, and then 1,2-dibromotetrachloroethane (71 mg, 0.22 mmol) was added. The mixture was warmed to room temperature and reacted sufficiently. A saturated aqueous ammonium chloride solution (3 mL) was added to the system, and ethyl acetate (3 mL×3) was added for extraction. The combined organic phases were washed with saturated brine, dried over anhydrous magnesium sulfate, filtered, and concentrated to dryness by rotary evaporation, and the residue was purified by flash column chromatography (petroleum ether:ethyl acetate=4:1) to give compound 64f (45.8 mg).

MS m/z (ESI): 265.2/267.2 [M+H]⁺.

### Step 5: Synthesis of Compound 64g

Compound 64g (17.5 mg) was obtained through synthesis and purification using a method identical to that described in step 2 of Example 7, with compound 7a (47 mg, 0.1 mmol) and 64f (27 mg, 0.1 mmol) as starting materials.

### Step 6: Synthesis of Compound 64

Compound 64 (8.0 mg) was obtained through synthesis and purification using a method identical to that described in step 8 of Example 4, with compound 64g (17 mg, 26 μmol) as starting material.

MS m/z (ESI): 538.3 [M+H]⁺.

¹H NMR (400 MHz, DMSO-d₆) δ 12.95 (s, 1H), 8.06 (d, J=8.6 Hz, 1H), 7.86-7.81 (m, 1H), 7.30 (dd, J=9.4, 1.6 Hz, 2H), 7.19 (dd, J=8.8, 7.4 Hz, 1H), 7.12 (dd, J=8.6, 2.1 Hz, 1H), 3.79 (t, J=6.4 Hz, 2H), 3.30 (t, J=6.5 Hz, 2H), 3.03 (s, 6H), 2.86-2.76 (m, 2H), 1.92-1.74 (m, 6H), 1.29-1.24 (m, 4H), 0.34 (s, 4H).

### Example 65: Synthesis of Compound 65

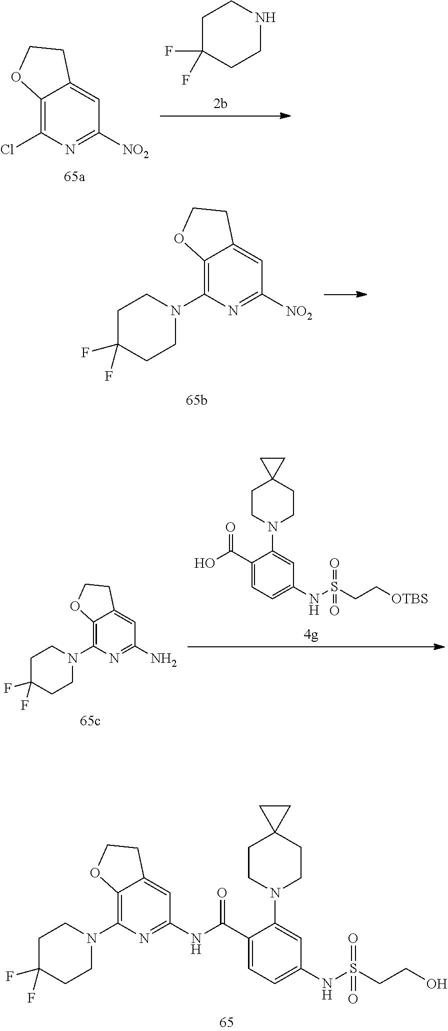 [SMILES: O=[N+]([O-])c1cc2c(c(Cl)n1)OCC2 ; FC1(F)CCNCC1 ; O=[N+]([O-])c1cc2c(c(N3CCC(F)(F)CC3)n1)OCC2 ; Nc1cc2c(c(N3CCC(F)(F)CC3)n1)OCC2 ; CC(C)(C)[Si](C)(C)OCCS(=O)(=O)Nc1ccc(C(=O)O)c(N2CCC3(CC2)CC3)c1 ; O=C(Nc1cc2c(c(N3CCC(F)(F)CC3)n1)OCC2)c1ccc(NS(=O)(=O)CCO)cc1N1CCC2(CC1)CC2]

### Step 1: Synthesis of Compound 65b

Compound 65b (17.8 mg) was obtained through synthesis and purification using a method identical to that described in step 1 of Example 30, with compound 65a (50 mg, 0.25 mmol) and 2b (242 mg, 2 mmol) as starting materials.

### Step 2: Synthesis of Compound 65c

Compound 65c (12 mg) was obtained through synthesis and purification using a method identical to that described in step 3 of Example 4, with compound 65b (17 mg, 0.063 mmol) as starting material.

MS m/z (ESI): 256.2 [M+H]⁺.

### Step 3: Synthesis of Compound 65

Compound 65 (18 mg) was obtained through synthesis and purification using a method identical to that described in step 5 of Example 23, with compound 65c (12 mg, 0.05 mmol) and compound 4g (24 mg, 0.05 mmol) as starting materials.

MS m/z (ESI): 592.5 [M+H]⁺.

¹H NMR (400 MHz, DMSO-d₆) δ 12.70 (s, 1H), 8.03 (d, J=8.4 Hz, 1H), 7.75 (s, 1H), 7.22 (s, 1H), 7.08 (d, J=8.8 Hz, 1H), 4.56 (t, J=8.8 Hz, 2H), 3.75 (t, J=6.8 Hz, 2H), 3.71-3.65 (m, 5H), 3.19 (t, J=8.8 Hz, 2H), 2.96 (t, J=8.4 Hz, 4H), 2.09-1.97 (m, 4H), 1.30-1.20 (m, 4H), 0.38-0.34 (m, 4H).

### Example 66: Synthesis of Compound 66

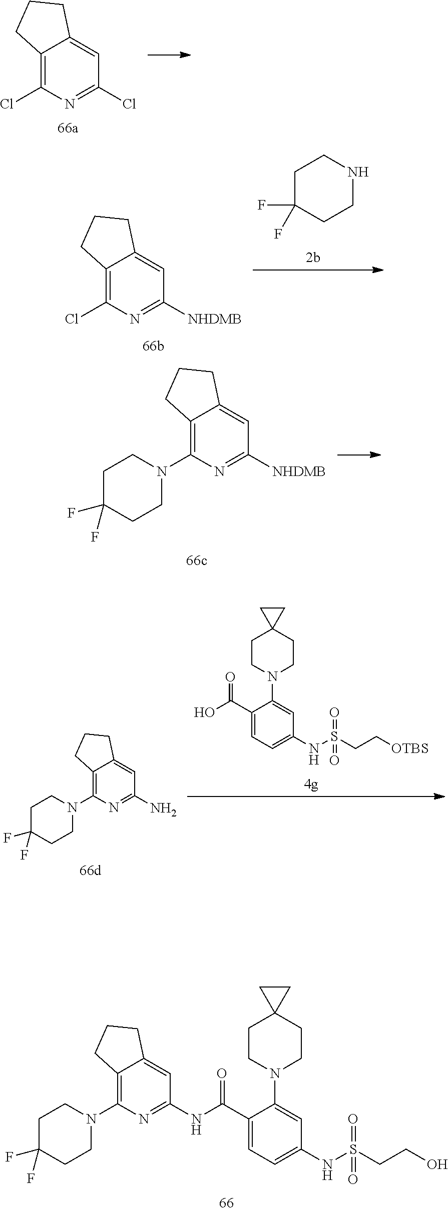

### Step 1: Synthesis of Compound 66b

Compound 66b (275 mg) was obtained through synthesis and purification using a method identical to that described in step 1 of Example 30, with compound 66a (200 mg, 1.06 mmol) and DMBNH₂ (217 mg, 1.3 mmol) as starting materials.

### Step 2: Synthesis of Compound 66c

Compound 66c (110 mg) was obtained through synthesis and purification using a method identical to that described in step 4 of Example 22, with compound 66b (200 mg, 0.63 mmol) and compound 2b (121 mg, 1.0 mmol) as starting materials.

### Step 3: Synthesis of Compound 66d

Compound 66d (56 mg) was obtained through synthesis and purification using a method identical to that described in step 5 of Example 30, with compound 66c (100 mg, 0.25 mmol) as starting material.

MS m/z (ESI): 254.1 [M+H]⁺.

### Step 6: Synthesis of Compound 66

Compound 66 (7.8 mg) was obtained through synthesis and purification using a method identical to that described in step 5 of Example 23, with compound 66d (25 mg, 0.1 mmol) and compound 4g (47 mg, 0.1 mmol) as starting materials.

MS m/z (ESI): 590.3 [M+H]⁺.

### Example 67: Synthesis of Compound 67

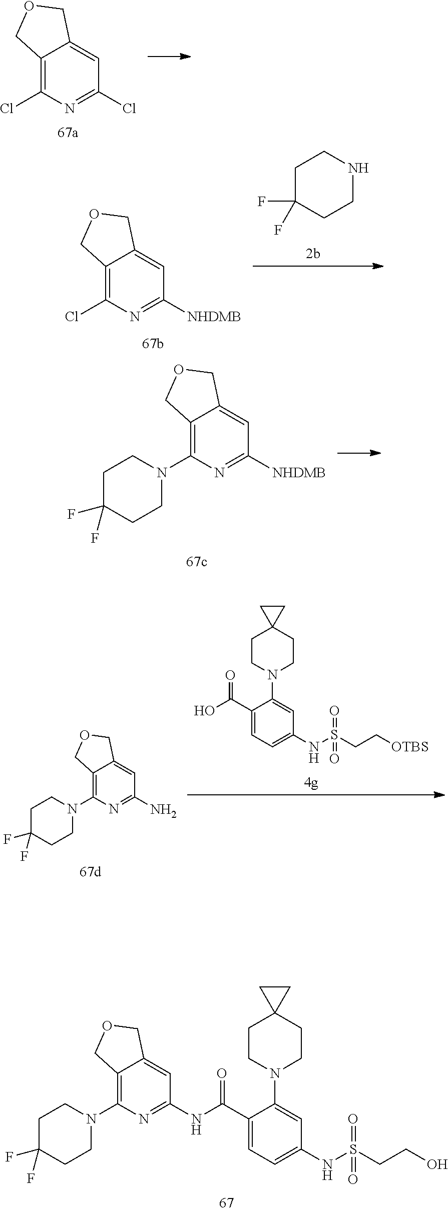

### Step 1: Synthesis of Compound 67b

Compound 67b (255 mg) was obtained through synthesis and purification using a method identical to that described in step 1 of Example 30, with compound 67a (200 mg, 1.05 mmol) and DMBNH₂ (217 mg, 1.3 mmol) as starting materials.

### Step 2: Synthesis of Compound 67c

Compound 67c (103 mg) was obtained through synthesis and purification using a method identical to that described in step 4 of Example 22, with compound 67b (200 mg, 0.62 mmol) and compound 2b (121 mg, 1.0 mmol) as starting materials.

### Step 3: Synthesis of Compound 67d

Compound 67d (38 mg) was obtained through synthesis and purification using a method identical to that described in step 5 of Example 30, with compound 67c (99 mg, 0.24 mmol) as starting material.

MS m/z (ESI): 256.1 [M+H]⁺.

### Step 6: Synthesis of Compound 67

Compound 67 (6.7 mg) was obtained through synthesis and purification using a method identical to that described in step 5 of Example 23, with compound 67d (26 mg, 0.1 mmol) and compound 4g (47 mg, 0.1 mmol) as starting materials.

MS m/z (ESI): 592.3 [M+H]⁺.

### Example 68: Synthesis of Compound 68

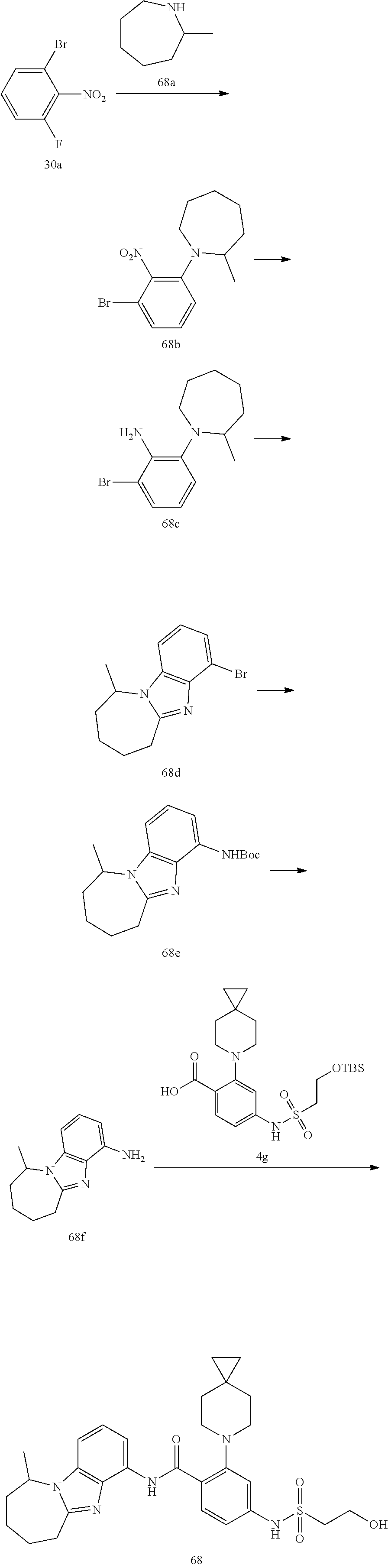

### Step 1: Synthesis of Compound 68b

Compound 68b (0.5 g) was obtained through synthesis and purification using a method identical to that described in step 1 of Example 30, with compound 30a (3.91 g, 17.8 mmol) and 68a (hydrochloride, 266 mg, 1.78 mmol) as starting materials.

### Step 2: Synthesis of Compound 68c

Compound 68c (0.32 g) was obtained through synthesis and purification using a method identical to that described in step 3 of Example 4, with compound 68b (0.5 g, 1.6 mmol) as starting material.

### Step 3: Synthesis of Compound 68d

Compound 68d (220 mg) was obtained through synthesis and purification using a method identical to that described in step 3 of Example 30, with compound 68c (300 mg, 1.06 mmol) as starting material.

### Step 4: Synthesis of Compound 68e

Compound 68e (180 mg) was obtained through synthesis and purification using a method identical to that described in step 4 of Example 30, with compound 68d (200 mg, 0.72 mmol) and tert-butyl carbamate (101 mg, 0.86 mmol) as starting materials.

### Step 5: Synthesis of Compound 68f

Compound 68f (90 mg) was obtained through synthesis and purification using a method identical to that described in step 5 of Example 30, with compound 68e (170 mg, 0.54 mmol) as starting material.

MS m/z (ESI): 216.1 [M+H]⁺.

### Step 6: Synthesis of Compound 68

Compound 68 (10.0 mg) was obtained through synthesis and purification using a method identical to that described in step 5 of Example 23, with compound 68f (43 mg, 0.2 mmol) and compound 4g (94 mg, 0.2 mmol) as starting materials.

MS m/z (ESI): 552.2 [M+H]⁺.

¹H NMR (400 MHz, DMSO-d₆) δ 12.49 (s, 1H), 8.32 (d, J=7.8 Hz, 1H), 8.05 (d, J=8.6 Hz, 1H), 7.32-7.21 (m, 2H), 7.16 (t, J=8.0 Hz, 1H), 7.09 (dd, J=8.6, 2.1 Hz, 1H), 4.99-4.88 (m, 1H), 3.77 (t, J=6.5 Hz, 2H), 3.74-3.65 (m, 2H), 3.61-3.57 (m, 1H), 3.35-3.23 (m, 2H), 3.00 (d, J=13.3 Hz, 5H), 2.15-2.09 (m, 1H), 2.06-1.75 (m, 7H), 1.46-1.42 (m, 3H), 0.31 (s, 4H).

### Example 69: Synthesis of Compound 69

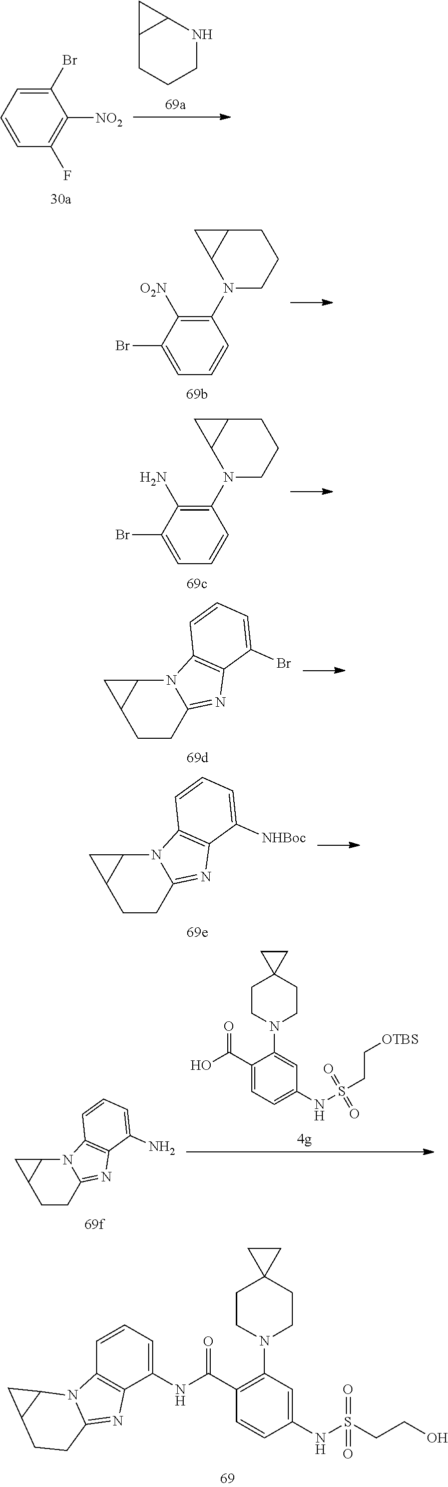

### Step 1: Synthesis of Compound 69b

Compound 69b (0.5 g) was obtained through synthesis and purification using a method identical to that described in step 1 of Example 30, with compound 30a (436 mg, 2.0 mmol) and 69a (hydrochloride, 240 mg, 1.8 mmol) as starting materials.

### Step 2: Synthesis of Compound 69c

Compound 69c (0.33 g) was obtained through synthesis and purification using a method identical to that described in step 3 of Example 4, with compound 69b (0.5 g, 1.7 mmol) as starting material.

### Step 3: Synthesis of Compound 69d

Compound 69d (170 mg) was obtained through synthesis and purification using a method identical to that described in step 3 of Example 30, with compound 69c (320 mg, 1.2 mmol) as starting material.

### Step 4: Synthesis of Compound 69e

Compound 69e (120 mg) was obtained through synthesis and purification using a method identical to that described in step 4 of Example 30, with compound 69d (160 mg, 0.61 mmol) and tert-butyl carbamate (86 mg, 0.73 mmol) as starting materials.

### Step 5: Synthesis of Compound 69f

Compound 69f (70 mg) was obtained through synthesis and purification using a method identical to that described in step 5 of Example 30, with compound 69e (110 mg, 0.37 mmol) as starting material.

MS m/z (ESI): 200.1 [M+H]⁺.

### Step 6: Synthesis of Compound 69

Compound 69 (18 mg) was obtained through synthesis and purification using a method identical to that described in step 5 of Example 23, with compound 69f (20 mg, 0.1 mmol) and compound 4g (60 mg, 0.13 mmol) as starting materials.

MS m/z (ESI): 536.1 [M+H]⁺.

¹H NMR (400 MHz, DMSO-d₆) δ 12.13 (s, 1H), 8.43 (dd, J=6.9, 2.0 Hz, 1H), 8.01 (d, J=8.6 Hz, 1H), 7.27-7.18 (m, 3H), 7.06 (dd, J=8.6, 2.1 Hz, 1H), 6.29 (d, J=1.9 Hz, 1H), 5.35 (d, J=1.9 Hz, 1H), 4.23 (t, J=5.9 Hz, 2H), 3.76 (t, J=6.5 Hz, 2H), 3.46-3.36 (m, 2H), 3.03 (t, J=5.3 Hz, 4H), 2.74 (t, J=5.9 Hz, 2H), 2.20-2.08 (m, 2H), 1.77 (s, 4H), 0.29 (s, 4H).

### Example 70 and Example 71: Synthesis of Compound 70 and Compound 71

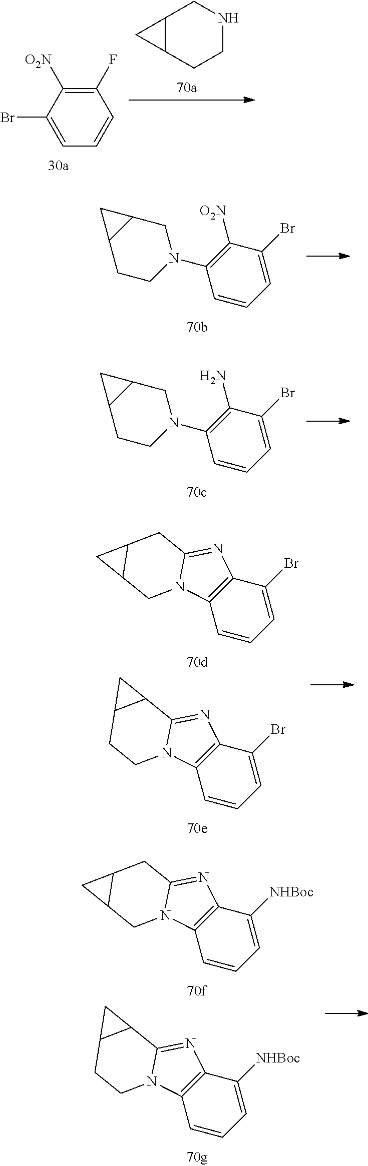

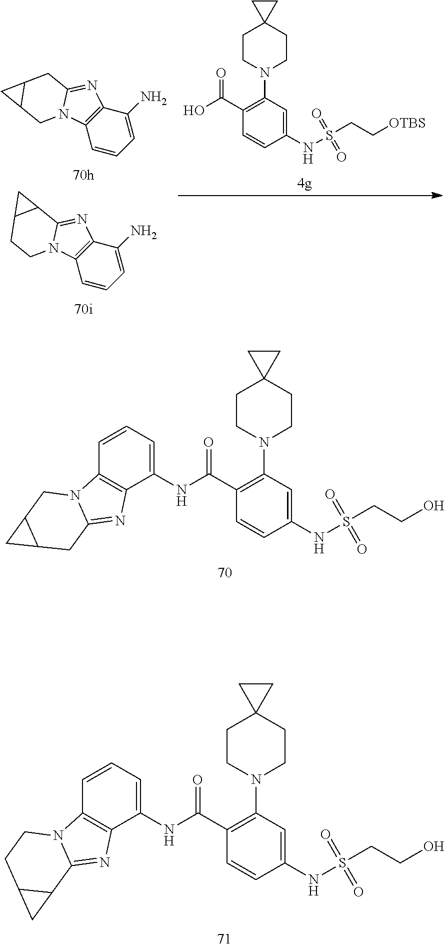 [SMILES: Nc1cccc2c1nc1n2CC2CC2C1 ; CC(C)(C)[Si](C)(C)OCCS(=O)(=O)Nc1ccc(C(=O)O)c(N2CCC3(CC2)CC3)c1 ; Nc1cccc2c1nc1n2CCC2CC12 ; O=C(Nc1cccc2c1nc1n2CC2CC2C1)c1ccc(NS(=O)(=O)CCO)cc1N1CCC2(CC1)CC2 ; O=C(Nc1cccc2c1nc1n2CCC2CC12)c1ccc(NS(=O)(=O)CCO)cc1N1CCC2(CC1)CC2]

### Step 1: Synthesis of Compound 70b

Compound 70b (0.5 g) was obtained through synthesis and purification using a method identical to that described in step 1 of Example 30, with compound 30a (436 mg, 2.0 mmol) and 70a (240 mg, 1.8 mmol) as starting materials.

### Step 2: Synthesis of Compound 70c

Compound 70c (0.39 g) was obtained through synthesis and purification using a method identical to that described in step 3 of Example 4, with compound 70b (0.5 g, 1.7 mmol) as starting material.

Step 3: Synthesis of Compound 70d and compound 70e

The mixture (280 mg) of compound 70d and compound 70e was obtained through synthesis and purification using a method identical to that described in step 3 of Example 30, with compound 70c (380 mg, 1.4 mmol) as starting material.

Step 4: Synthesis of Compound 70f and compound 70g

The mixture (240 mg) of compound 70f and compound 70g was obtained through synthesis and purification using a method identical to that described in step 4 of Example 30, with the mixture (260 mg, 1.0 mmol) of compound 70d and compound 70e and tert-butyl carbamate (140 mg, 1.2 mmol) as starting materials.

Step 5: Synthesis of Compound 70 h and compound 70i

The mixture (95 mg) of compound 70 h and compound 70i was obtained through synthesis and purification using a method identical to that described in step 5 of Example 30, with the mixture (230 mg, 0.77 mmol) of compound 70f and compound 70g as starting material.

MS m/z (ESI): 200.1 [M+H]⁺.

Step 6: Synthesis of Compound 70 and compound 71

The final products were obtained through synthesis and purification using a method identical to that described in step 5 of Example 23, with the mixture (80 mg, 0.4 mmol) of compound 70 h and compound 70i and compound 4g (234 mg, 0.5 mmol) as starting materials.

Compound 70 (6.0 mg)

MS m/z (ESI): 536.2 [M+H]⁺.

¹H NMR (400 MHz, DMSO-d₆) δ 12.44 (s, 1H), 9.94 (s, 1H), 8.32 (dd, J=7.7, 1.2 Hz, 1H), 8.03 (d, J=8.6 Hz, 1H), 7.26 (d, J=2.1 Hz, 1H), 7.20 (dd, J=8.1, 1.3 Hz, 1H), 7.15 (t, J=7.9 Hz, 1H), 7.08 (dd, J=8.6, 2.1 Hz, 1H), 4.96 (s, 1H), 4.59 (d, J=12.8 Hz, 1H), 4.30 (dd, J=12.7, 2.9 Hz, 1H), 3.76 (t, J=6.5 Hz, 2H), 3.38-3.35 (m, 2H), 2.97 (t, J=5.4 Hz, 4H), 2.08-1.65 (m, 4H), 1.61-1.52 (m, 2H), 0.75-0.64 (m, 1H), 0.30 (s, 4H), −0.03-−0.13 (m, 1H).

Compound 71 (40 mg)

MS m/z (ESI): 536.2 [M+H]⁺.

¹H NMR (400 MHz, DMSO-d₆) δ 12.46 (s, 1H), 10.11 (s, 1H), 8.30 (dd, J=7.3, 1.7 Hz, 1H), 8.04 (d, J=8.6 Hz, 1H), 7.28 (d, J=2.2 Hz, 1H), 7.18-7.05 (m, 3H), 4.95 (s, 1H), 4.31 (dd, J=13.1, 6.0 Hz, 1H), 3.77 (t, J=6.5 Hz, 2H), 3.69 (td, J=12.9, 4.8 Hz, 1H), 3.36 (t, J=6.6 Hz, 2H), 3.00 (t, J=5.5 Hz, 4H), 2.37-2.22 (m, 2H), 2.18-1.98 (m, 2H), 1.98-1.62 (m, 4H), 1.30-1.10 (m, 2H), 0.32 (s, 4H).

### Example 72: Synthesis of Compound 72

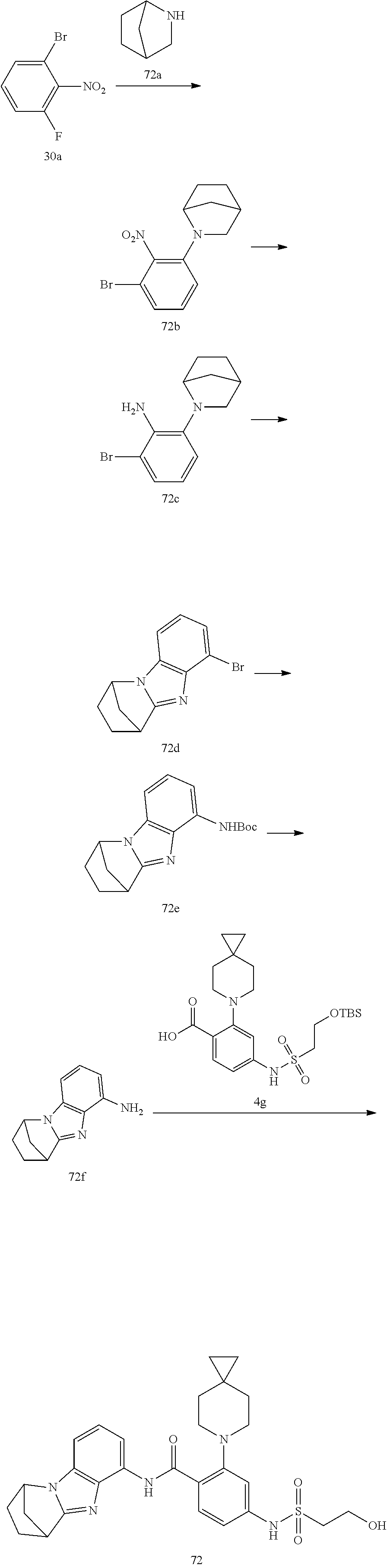

### Step 1: Synthesis of Compound 72b

Compound 72b (0.3 g) was obtained through synthesis and purification using a method identical to that described in step 1 of Example 30, with compound 30a (224 mg, 1.0 mmol) and 72a (100 mg, 1.0 mmol) as starting materials.

### Step 2: Synthesis of Compound 72c

Compound 72c (0.26 g) was obtained through synthesis and purification using a method identical to that described in step 3 of Example 4, with compound 72b (0.3 g, 1.0 mmol) as starting material.

### Step 3: Synthesis of Compound 72d

Compound 72d (110 mg) was obtained through synthesis and purification using a method identical to that described in step 3 of Example 30, with compound 72c (0.26 g, 1.0 mmol) as starting material.

### Step 4: Synthesis of Compound 72e

Compound 72e (110 mg) was obtained through synthesis and purification using a method identical to that described in step 4 of Example 30, with compound 72d (100 mg, 0.38 mmol) and tert-butyl carbamate (89 mg, 0.76 mmol) as starting materials.

### Step 5: Synthesis of Compound 72f

Compound 72f (50 mg) was obtained through synthesis and purification using a method identical to that described in step 5 of Example 30, with compound 72e (110 mg, 0.37 mmol) as starting material.

MS m/z (ESI): 200.1 [M+H]⁺.

### Step 6: Synthesis of Compound 72

Compound 72 (12.5 mg) was obtained through synthesis and purification using a method identical to that described in step 5 of Example 23, with compound 72f (20 mg, 0.1 mmol) and compound 4g (47 mg, 0.1 mmol) as starting materials.

MS m/z (ESI): 536.2 [M+H]⁺.

¹H NMR (400 MHz, DMSO-d₆) δ 12.33 (s, 1H), 8.28 (d, J=7.8 Hz, 1H), 8.00 (d, J=8.6 Hz, 1H), 7.27-7.21 (m, 2H), 7.13 (t, J=8.0 Hz, 1H), 7.07 (dd, J=8.6, 2.1 Hz, 1H), 5.15 (s, 1H), 3.76 (t, J=6.5 Hz, 2H), 3.59 (d, J=3.7 Hz, 1H), 3.35-3.32 (m, 2H), 3.05-2.91 (m, 4H), 2.27-2.21 (m, 1H), 2.19-2.09 (m, 1H), 2.03-1.95 (m, 2H), 1.74 (s, 4H), 1.20-1.09 (m, 2H), 0.29 (s, 4H).

### Example 73: Synthesis of Compound 73

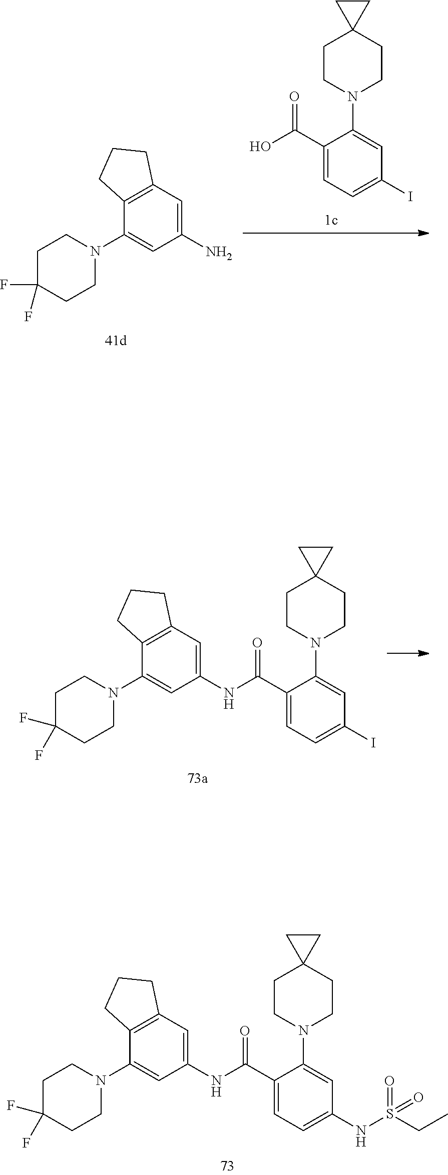 [SMILES: Nc1cc2c(c(N3CCC(F)(F)CC3)c1)CCC2 ; O=C(O)c1ccc(I)cc1N1CCC2(CC1)CC2 ; O=C(Nc1cc2c(c(N3CCC(F)(F)CC3)c1)CCC2)c1ccc(I)cc1N1CCC2(CC1)CC2 ; CCS(=O)(=O)Nc1ccc(C(=O)Nc2cc3c(c(N4CCC(F)(F)CC4)c2)CCC3)c(N2CCC3(CC2)CC3)c1]

### Step 1: Synthesis of Compound 73a

Compound 73a (27 mg) was obtained through synthesis and purification using a method identical to that described in step 2 of Example 1, with compound 41d (25 mg, 0.1 mmol) and compound 1c (36 mg, 0.1 mmol) as starting materials.

### Step 2: Synthesis of Compound 73

Compound 73 (7 mg) was obtained through synthesis and purification using a method identical to that described in step 3 of Example 1, with compound 73a (24 mg, 0.04 mmol) and ethanesulfonamide (11 mg, 0.1 mmol) as starting materials.

MS m/z (ESI): 573.2 [M+H]⁺.

¹H NMR (400 MHz, DMSO-d₆) δ 11.65 (s, 1H), 10.13 (br, 1H), 7.83 (d, J=8.5 Hz, 1H), 7.46 (s, 1H), 7.17 (d, J=2.2 Hz, 1H), 7.08 (s, 1H), 7.04 (dd, J=8.5 Hz, J=2.1 Hz, 1H), 3.19 (q, J=7.3 Hz, 2H), 3.07 (t, J=5.5 Hz, 4H), 2.96 (t, J=5.3 Hz, 4H), 2.84 (t, J=7.3 Hz, 2H), 2.77 (t, J=7.2 Hz, 2H), 2.12 (m, 4H), 2.03-1.95 (m, 2H), 1.56 (br, 4H), 1.21 (t, J=7.3 Hz, 2H), 0.35 (s, 4H).

### Example 74: Synthesis of Compound 74

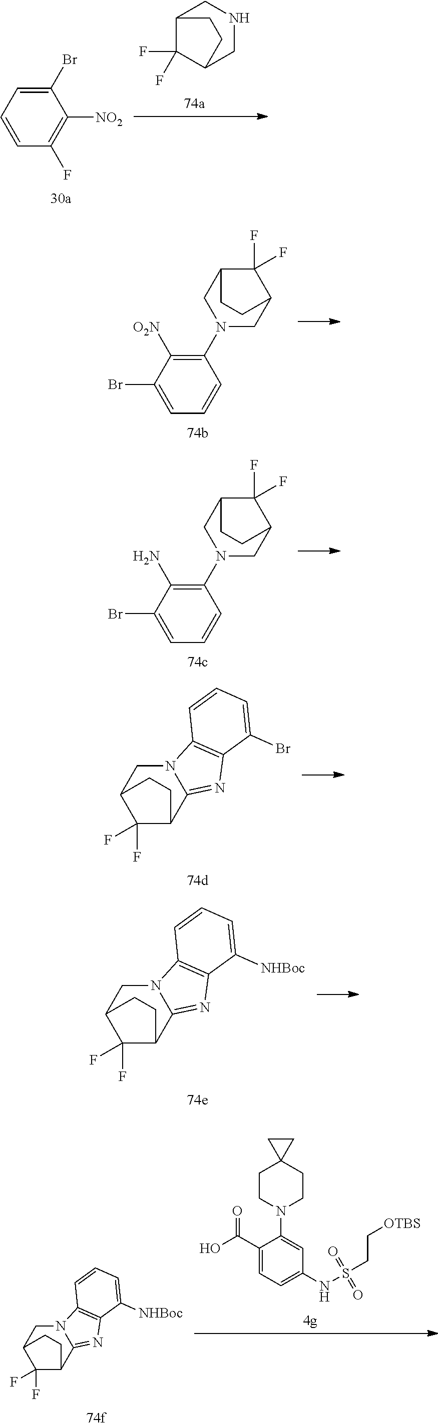

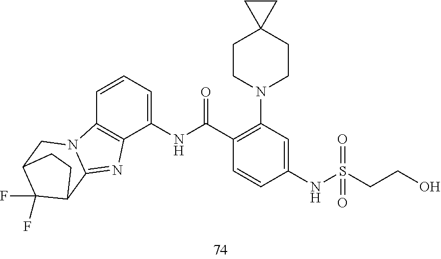 [SMILES: O=C(Nc1cccc2c1nc1n2CC2CCC1C2(F)F)c1ccc(NS(=O)(=O)CCO)cc1N1CCC2(CC1)CC2]

### Step 1: Synthesis of Compound 74b

Compound 74b (453 mg) was obtained through synthesis and purification using a method identical to that described in step 1 of Example 30, with compound 30a (414 mg, 1.88 mmol) and 74a (294 mg, 2.0 mmol) as starting materials.

### Step 2: Synthesis of Compound 74c

Compound 74c (150 mg) was obtained through synthesis and purification using a method identical to that described in step 3 of Example 4, with compound 74b (347 mg, 1.0 mmol) as starting material.

### Step 3: Synthesis of Compound 74d

Compound 74d (90 mg) was obtained through synthesis and purification using a method identical to that described in step 3 of Example 30, with compound 74c (150 mg, 0.47 mmol) as starting material.

### Step 4: Synthesis of Compound 74e

Compound 74e (77 mg) was obtained through synthesis and purification using a method identical to that described in step 4 of Example 30, with compound 74d (80 mg, 0.25 mmol) and tert-butyl carbamate (59 mg, 0.5 mmol) as starting materials.

### Step 5: Synthesis of Compound 74f

Compound 74f (50 mg) was obtained through synthesis and purification using a method identical to that described in step 5 of Example 30, with compound 74e (77 mg, 0.22 mmol) as starting material.

MS m/z (ESI): 200.1 [M+H]⁺.

### Step 6: Synthesis of Compound 74

Compound 74 (20 mg) was obtained through synthesis and purification using a method identical to that described in step 5 of Example 23, with compound 74f (40 mg, 0.16 mmol) and compound 4g (75 mg, 0.16 mmol) as starting materials.

MS m/z (ESI): 586.2 [M+H]⁺.

¹H NMR (400 MHz, DMSO-d₆) δ 12.51 (s, 1H), 10.11 (s, 1H), 8.38 (dd, J=7.2, 1.7 Hz, 1H), 8.03 (d, J=8.5 Hz, 1H), 7.27-7.17 (m, 3H), 7.07 (dd, J=8.6, 2.0 Hz, 1H), 4.92 (s, 1H), 4.34 (d, J=12.2 Hz, 1H), 4.29-4.21 (m, 1H), 3.76 (t, J=6.5 Hz, 2H), 3.72-3.65 (m, 1H), 3.37-3.33 (m, 2H), 3.11-3.05 (m, 1H), 3.04-2.90 (m, 4H), 2.30-2.21 (m, 2H), 1.98-1.90 (m, 2H), 1.90-1.58 (m, 4H), 0.30 (s, 4H).

### Example 75: Synthesis of Compound 75

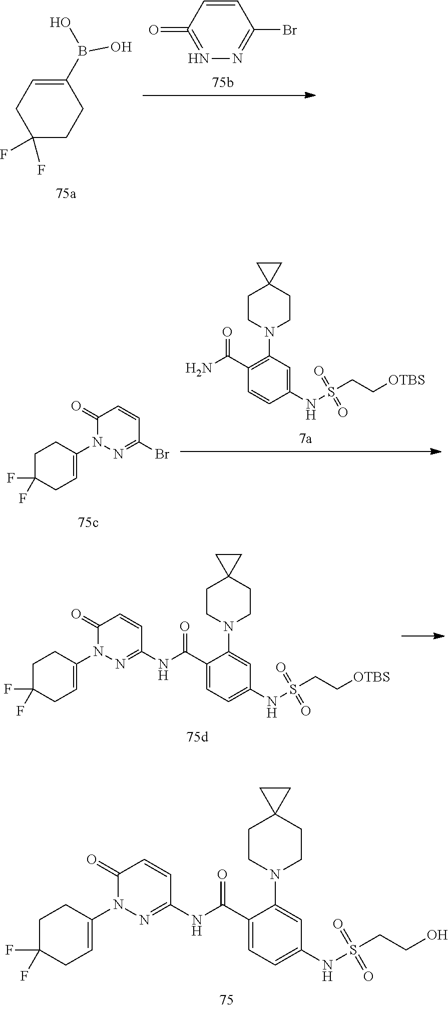

### Step 1: Synthesis of Compound 75c

Copper acetate (11.4 mg, 57 μmol) and pyridine (45.2 mg, 571 μmol) were added to a solution of 75a (60.2 mg, 371 μmol) and 75b (50 mg, 285 μmol) in DMF (2 mL), and the mixture was stirred and reacted sufficiently at 100 °C in an oxygen atmosphere. The reaction mixture was filtered, the volatiles were removed under vacuum, and the residue was purified by flash column chromatography (petroleum ether:ethyl acetate=3:1) to give compound 75c (61 mg).

### Step 2: Synthesis of Compound 75d

Compound 75d (9 mg) was obtained through synthesis and purification using a method identical to that described in step 2 of Example 7, with compound 75c (25 mg, 86 μmol) and 7a (40 mg, 86 μmol) as starting materials.

### Step 3: Synthesis of Compound 75

Compound 75 (2.4 mg) was obtained through synthesis and purification using a method identical to that described in step 8 of Example 4, with compound 75d (9 mg) as starting material.

MS m/z (ESI): 564.2 [M+H]⁺.

¹H NMR (400 MHz, DMSO-d₆) δ 13.63 (s, 1H), 8.48-8.15 (m, 1H), 7.98 (d, J=8.6 Hz, 1H), 7.20 (s, 1H), 7.13-7.05 (m, 2H), 6.09 (s, 1H), 3.75 (t, J=6.4 Hz, 2H), 3.30-3.29 (m, 2H), 2.98-2.93 (m, 4H), 2.80 (t, J=14.7 Hz, 2H), 2.71-2.66 (m, 2H), 2.27-2.16 (m, 2H), 1.65 (s, 4H), 0.38 (s, 4H).

### Example 76: Synthesis of Compound 76

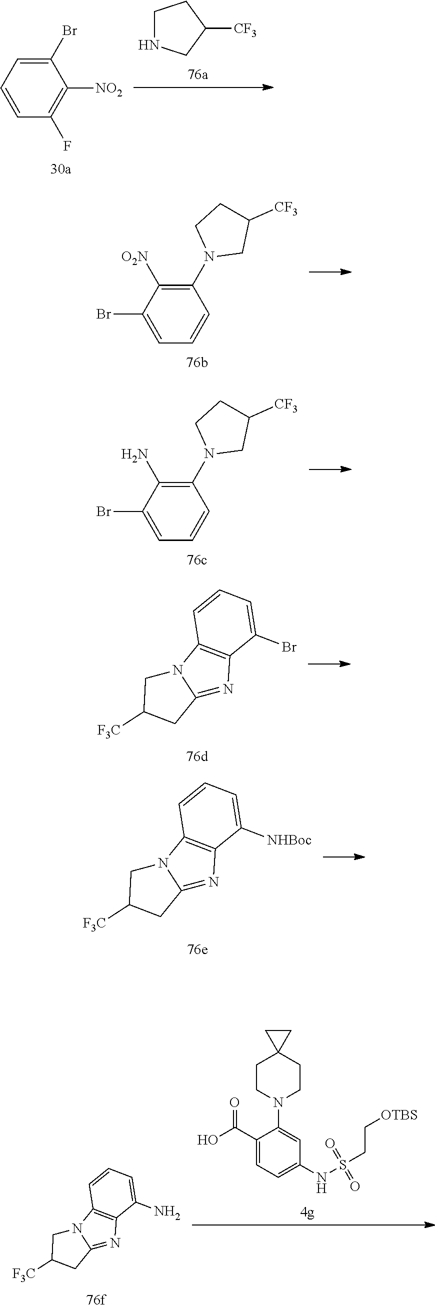

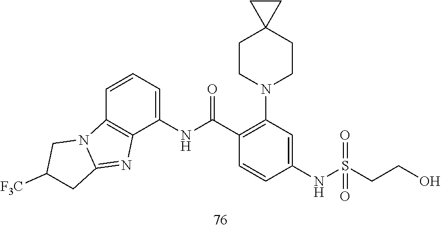 [SMILES: O=C(Nc1cccc2c1nc1n2CC(C(F)(F)F)C1)c1ccc(NS(=O)(=O)CCO)cc1N1CCC2(CC1)CC2]

### Step 1: Synthesis of Compound 76b

Compound 76b (585 mg) was obtained through synthesis and purification using a method identical to that described in step 1 of Example 30, with compound 30a (760 mg, 3.45 mmol) and 76a (240 mg, 1.73 mmol) as starting materials.

### Step 2: Synthesis of Compound 76c

Compound 76c (349 mg) was obtained through synthesis and purification using a method identical to that described in step 3 of Example 4, with compound 76b (400 mg, 1.18 mmol) as starting material.

### Step 3: Synthesis of Compound 76d

Compound 76d (250 mg) was obtained through synthesis and purification using a method identical to that described in step 3 of Example 30, with compound 76c (330 mg, 1.07 mmol) as starting material.

### Step 4: Synthesis of Compound 76e

Compound 76e (240 mg) was obtained through synthesis and purification using a method identical to that described in step 4 of Example 30, with compound 76d (242 mg, 0.79 mmol) and tert-butyl carbamate (230 mg, 2.0 mmol) as starting materials.

### Step 5: Synthesis of Compound 76f

Compound 76f (70 mg) was obtained through synthesis and purification using a method identical to that described in step 5 of Example 30, with compound 76e (240 mg, 0.70 mmol) as starting material.

MS m/z (ESI): 242.1 [M+H]⁺.

### Step 6: Synthesis of Compound 76

Compound 76 (5 mg) was obtained through synthesis and purification using a method identical to that described in step 5 of Example 23, with compound 76f (62 mg, 0.26 mmol) and compound 4g (80 mg, 0.17 mmol) as starting materials.

MS m/z (ESI): 578.1 [M+H]⁺.

¹H NMR (400 MHz, DMSO-d₆) of δ 12.38 (s, 1H), 8.34 (dd, J=7.7, 1.3 Hz, 1H), 8.00 (d, J=8.6 Hz, 1H), 7.25-7.21 (m, 2H), 7.18 (t, J=7.9 Hz, 1H), 7.05 (dd, J=8.6, 2.1 Hz, 1H), 4.53 (dd, J=11.2, 8.8 Hz, 1H), 4.33 (dd, J=11.2, 4.8 Hz, 1H), 4.28-4.17 (m, 1H), 3.76 (t, J=6.5 Hz, 2H), 3.43 (dd, J=17.1, 9.6 Hz, 1H), 3.35-3.33 (m, 2H), 3.08 (dd, J=17.1, 5.1 Hz, 1H), 3.04-2.92 (m, 4H), 1.98-1.54 (m, 4H), 0.29 (s, 4H).

### Example 77: Synthesis of Compound 77

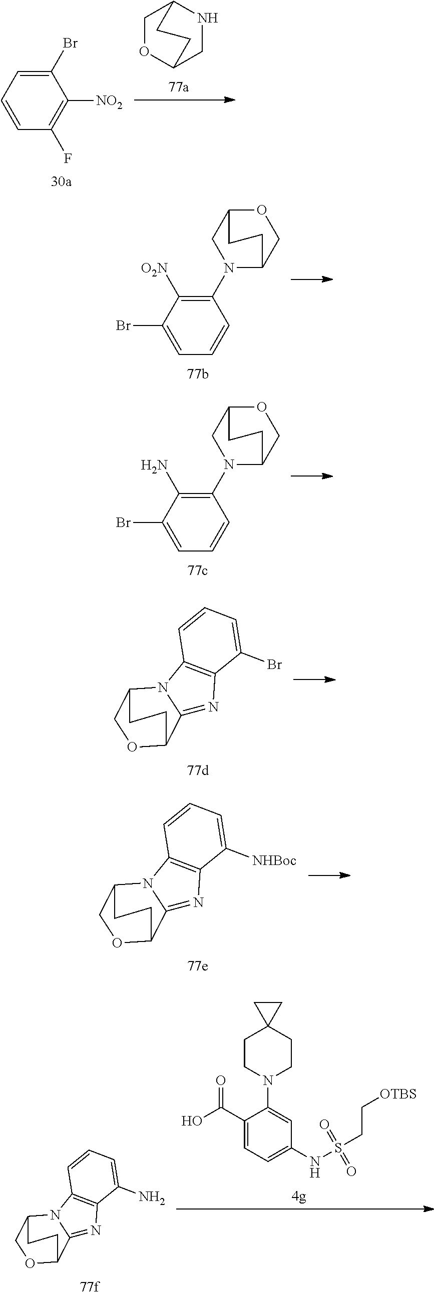

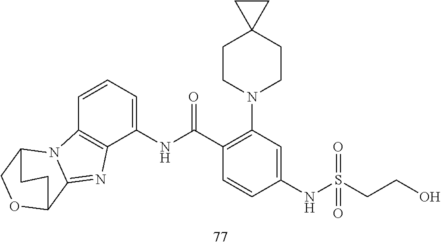 [SMILES: O=C(Nc1cccc2c1nc1n2C2CCC1OC2)c1ccc(NS(=O)(=O)CCO)cc1N1CCC2(CC1)CC2]

### Step 1: Synthesis of Compound 77b

Compound 77b (358 mg) was obtained through synthesis and purification using a method identical to that described in step 1 of Example 30, with compound 30a (440 mg, 2.0 mmol) and 77a (226 mg, 2.0 mmol) as starting materials.

### Step 2: Synthesis of Compound 77c

Compound 77c (219 mg) was obtained through synthesis and purification using a method identical to that described in step 3 of Example 4, with compound 77b (310 mg, 1.0 mmol) as starting material.

### Step 3: Synthesis of Compound 77d

Compound 77d (150 mg) was obtained through synthesis and purification using a method identical to that described in step 3 of Example 30, with compound 77c (200 mg, 0.71 mmol) as starting material.

### Step 4: Synthesis of Compound 77e

Compound 77e (105 mg) was obtained through synthesis and purification using a method identical to that described in step 4 of Example 30, with compound 77d (140 mg, 0.5 mmol) and tert-butyl carbamate (100 mg, 1.0 mmol) as starting materials.

### Step 5: Synthesis of Compound 77f

Compound 77f (70 mg) was obtained through synthesis and purification using a method identical to that described in step 5 of Example 30, with compound 77e (100 mg, 0.32 mmol) as starting material.

MS m/z (ESI): 216.1 [M+H]⁺.

### Step 6: Synthesis of Compound 77

Compound 77 (8 mg) was obtained through synthesis and purification using a method identical to that described in step 5 of Example 23, with compound 77f (22 mg, 0.1 mmol) and compound 4g (47 mg, 0.1 mmol) as starting materials.

MS m/z (ESI): 552.2 [M+H]⁺.

### Example 78: Synthesis of Compound 78

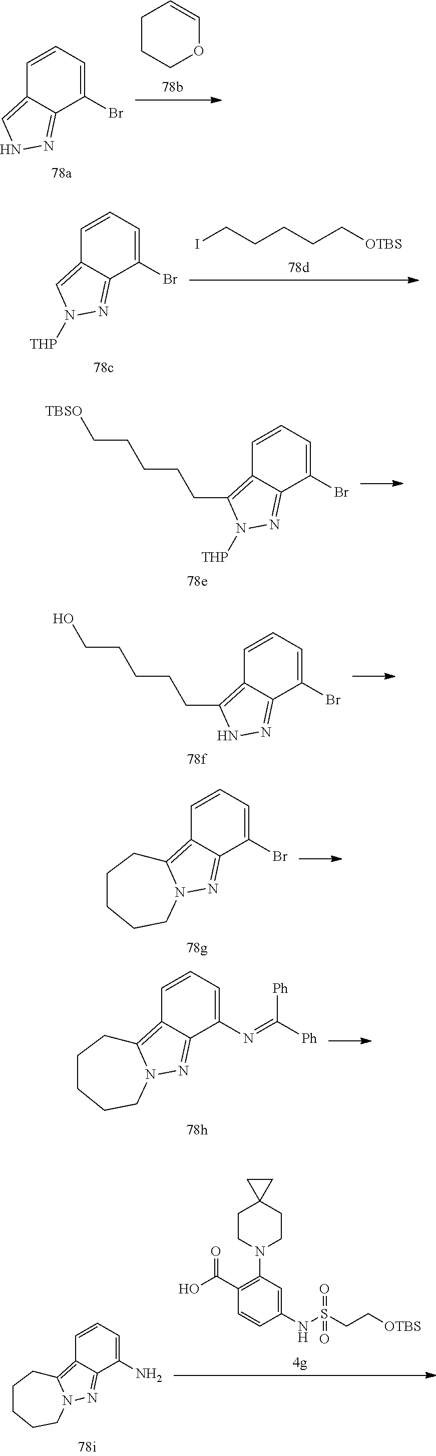

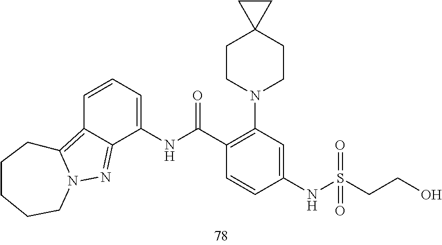 [SMILES: O=C(Nc1cccc2c3n(nc12)CCCCC3)c1ccc(NS(=O)(=O)CCO)cc1N1CCC2(CC1)CC2]

### Step 1: Synthesis of Compound 78c

Compound 78a (300 mg, 1.53 mmol) and compound 78b (193 mg, 2.29 mmol) were added to dichloromethane (3 mL), and the mixture was reacted sufficiently at room temperature. Water (5 mL) was added to the system, and dichloromethane (3 mL×3) was added for extraction. The combined organic phases were washed with saturated brine, dried over anhydrous magnesium sulfate, filtered, and concentrated to dryness by rotary evaporation, and the residue was purified by flash column chromatography (petroleum ether:ethyl acetate=8:1) to give compound 78c (373 mg).

### Step 2: Synthesis of Compound 78e

Compound 78c (373 mg, 1.33 mmol) was added to THF (6 mL), followed by the addition of lithium diisopropylamide (2 mL, 2.00 mmol, 1 N) at −60 °C. The mixture was reacted at this temperature for 10 min, and then 78d (655 mg, 2.00 mmol) was added. The mixture was warmed to room temperature and reacted sufficiently. A saturated ammonium chloride solution (10 mL) was added to the system, and ethyl acetate (5 mL×3) was added for extraction. The combined organic phases were washed with saturated brine, dried over anhydrous magnesium sulfate, and purified by flash column chromatography (petroleum ether:ethyl acetate=5:1) to give compound 78e (331 mg).

### Step 3: Synthesis of Compound 78f

Compound 78e (331 mg, 678 μmol) was added to dioxane (1 mL), followed by the addition of a solution of hydrochloric acid in dioxane (1.70 mL, 6.78 mmol, 4 N). The mixture was reacted sufficiently at room temperature, and then pyridine hydrofluoride (336 mg, 3.39 mmol) was added. The mixture was reacted sufficiently at room temperature. The volatiles were removed under vacuum, and the residue was purified by flash reverse-phase column chromatography (acetonitrile:water) to give compound 78f (173 mg).

### Step 4: Synthesis of Compound 78g

Compound 78f (173 mg, 603 μmol) was added to tetrahydrofuran (20 mL), followed by the addition of triphenylphosphine (474 mg, 1.8 mmol) and diisopropyl azodicarboxylate (DIAD, 366 mg, 1.8 mmol). The mixture was reacted sufficiently at room temperature. The reaction mixture was filtered and concentrated to dryness by rotary evaporation, and the residue was purified by flash column chromatography (petroleum ether:ethyl acetate=2:1) to give compound 78g (79.5 mg).

### Step 5: Synthesis of Compound 78h

Compound 78h (56.9 mg) was obtained through synthesis and purification using a method identical to that described in step 4 of Example 30, with compound 78g (72.3 mg, 274 μmol) as starting material.

### Step 6: Synthesis of Compound 78i

Compound 78i (21 mg) was obtained through synthesis and purification using a method identical to that described in step 5 of Example 30, with compound 78h (56.9 mg, 156 μmol) as starting material.

MS m/z (ESI): 202.1 [M+H]⁺.

### Step 7: Synthesis of Compound 78

Compound 78 (4 mg) was obtained through synthesis and purification using a method identical to that described in step 5 of Example 23, with compound 78i (20 mg, 43 μmol) and compound 4g (31 mg, 67 mol) as starting materials.

MS m/z (ESI): 538.5 [M+H]⁺.

### Example 79: Synthesis of Compound 79

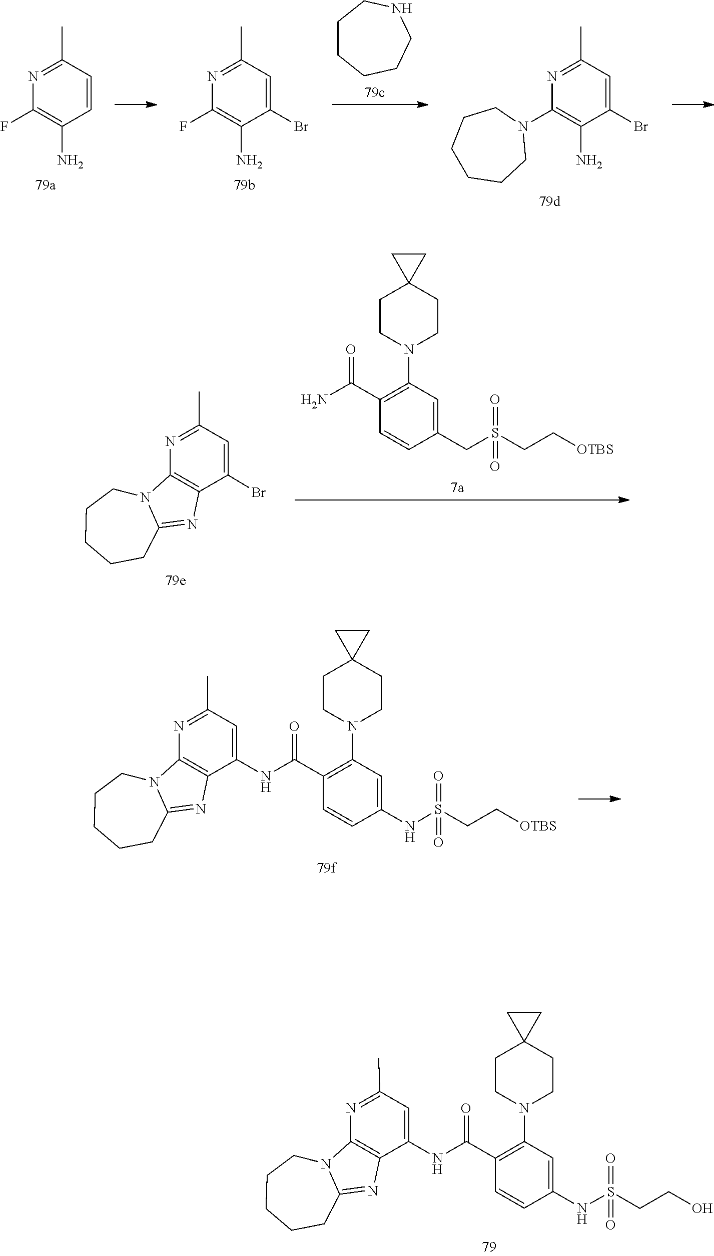

### Step 1: Synthesis of Compound 79b

Compound 79a (170 mg, 1.36 mmol) was added to methanol (2 mL) and acetic acid (0.2 mL), followed by the addition of bromine (260 mg, 1.63 mmol). The mixture was reacted sufficiently at room temperature. A saturated aqueous sodium bicarbonate solution (6 mL) was added to the system, and ethyl acetate (3 mL×3) was added for extraction. The combined organic phases were washed with saturated brine, dried over anhydrous magnesium sulfate, filtered, and concentrated to dryness by rotary evaporation, and the residue was purified by flash column chromatography (petroleum ether:ethyl acetate=1:1) to give compound 79b (254 mg).

### Step 2: Synthesis of Compound 79d

Compound 79d (241 mg) was obtained through synthesis and purification using a method identical to that described in step 1 of Example 30, with compound 79b (254 mg, 1.18 mmol) and compound 79c (141 mg, 1.42 mmol) as starting materials.

### Step 3: Synthesis of Compound 79e

Compound 79e (93 mg) was obtained through synthesis and purification using a method identical to that described in step 3 of Example 30, with compound 79d (241 mg, 0.85 mmol) as starting material.

### Step 4: Synthesis of Compound 79f

Compound 79f (26 mg) was obtained through synthesis and purification using a method identical to that described in step 2 of Example 7, with compound 7a (48 mg, 0.1 mmol) and 79e (28 mg, 0.1 mmol) as starting materials.

### Step 5: Synthesis of Compound 79

Compound 79 (5.4 mg) was obtained through synthesis and purification using a method identical to that described in step 8 of Example 4, with compound 79f (26 mg, 39 μmol) as starting material.

MS m/z (ESI): 553.4 [M+H]⁺.

### Example 80: Synthesis of Compound 80

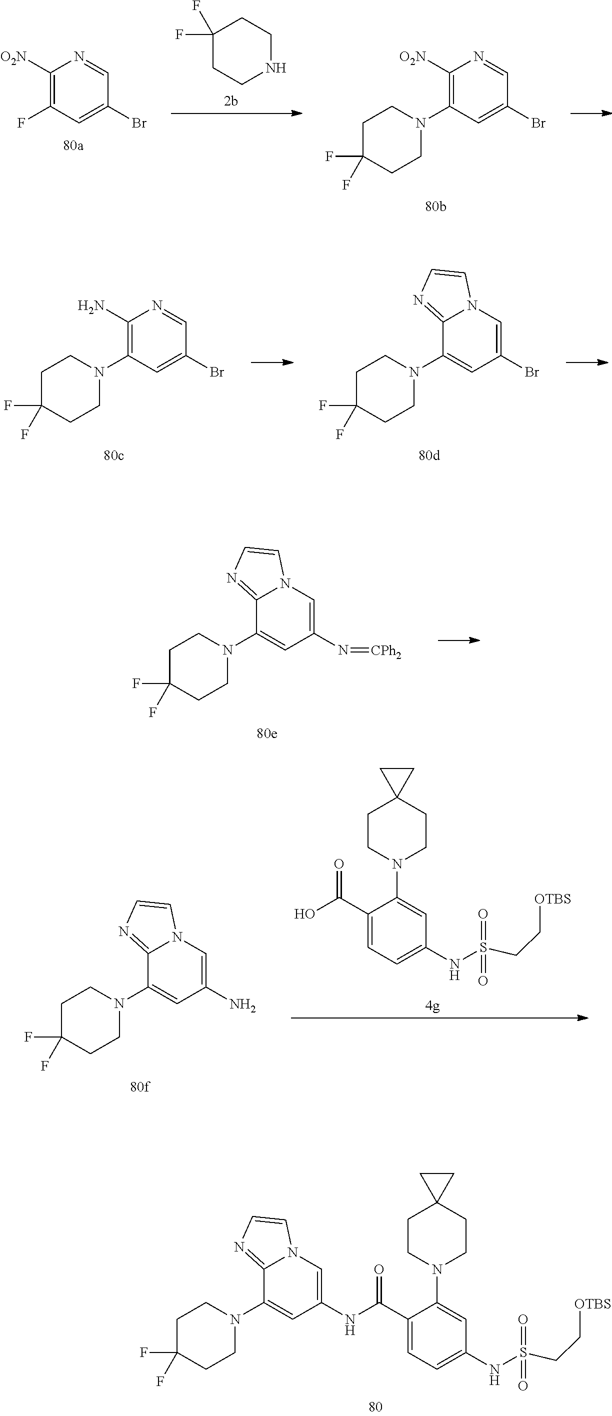

### Step 1: Synthesis of Compound 80b

Compound 80b (273 mg) was obtained through synthesis and purification using a method identical to that described in step 1 of Example 30, with compound 80a (220 mg, 1.0 mmol) and compound 2b (242 mg, 2.0 mmol) as starting materials.

### Step 2: Synthesis of Compound 80c

Compound 80c (182 mg) was obtained through synthesis and purification using a method identical to that described in step 3 of Example 4, with compound 80b (257 mg, 0.8 mmol) as starting material.

### Step 3: Synthesis of Compound 80d

Compound 80d (117 mg) was obtained through synthesis and purification using a method identical to that described in step 1 of Example 22, with compound 80c (145 mg, 0.5 mmol) as starting material.

### Step 4: Synthesis of Compound 80e

Compound 80e (39.4 mg) was obtained through synthesis and purification using a method identical to that described in step 4 of Example 30, with compound 80d (63 mg, 0.2 mmol) and benzophenone imine (40 mg, 0.22 mmol) as starting materials.

### Step 5: Synthesis of Compound 80f

Compound 80f (16.1 mg) was obtained through synthesis and purification using a method identical to that described in step 5 of Example 30, with compound 80e (33 mg, 0.08 mmol) as starting material.

MS m/z (ESI): 253.1 [M+H]⁺.

### Step 6: Synthesis of Compound 80

Compound 80 (9.4 mg) was obtained through synthesis and purification using a method identical to that described in step 5 of Example 23, with compound 80f (12.6 mg, 0.05 mmol) and compound 4g (47 mg, 0.1 mmol) as starting materials.

MS m/z (ESI): 589.3 [M+H]⁺.

### Example 81: Synthesis of Compound 81

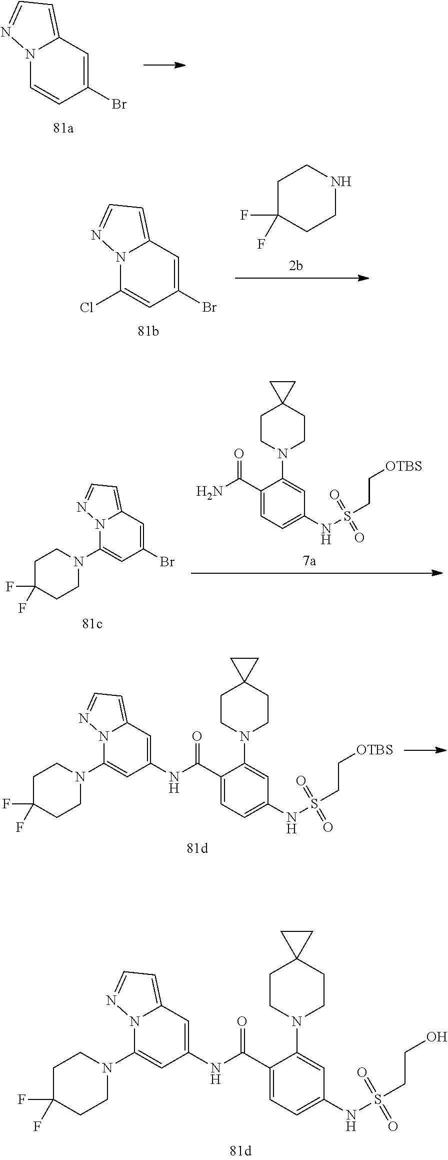

### Step 1: Synthesis of Compound 81b

Compound 81a (100 mg, 0.51 mmol) was added to THE (1 mL), and then the mixture was cooled to −78 °C, followed by the slow and dropwise addition of n-butyllithium (0.48 mL, 1.6 M, 0.76 mmol) and the addition of hexachloroethane (0.15 g, 0.61 mmol). The mixture was warmed to room temperature and reacted sufficiently. The volatiles were removed under vacuum, and the residue was directly separated by flash column chromatography to give compound 81b (76.2 mg).

### Step 2: Synthesis of Compound 81c

Compound 81c (76.2 mg) was obtained through synthesis and purification using a method identical to that described in step 1 of Example 30, with compound 81b (50 mg, 0.22 mmol) and compound 2b (266 mg, 2.2 mmol) as starting materials.

### Step 3: Synthesis of Compound 81d

Compound 81d (21 mg) was obtained through synthesis and purification using a method identical to that described in step 2 of Example 7, with compound 7a (26 mg, 0.055 mmol) and 81c (16 mg, 0.05 mmol) as starting materials.

### Step 4: Synthesis of Compound 81

Compound 81 (7.6 mg) was obtained through synthesis and purification using a method identical to that described in step 8 of Example 4, with compound 81d (21 mg, 30 μmol) as starting material.

MS m/z (ESI): 589.3 [M+H]⁺.

### Example 82: Synthesis of Compound 82

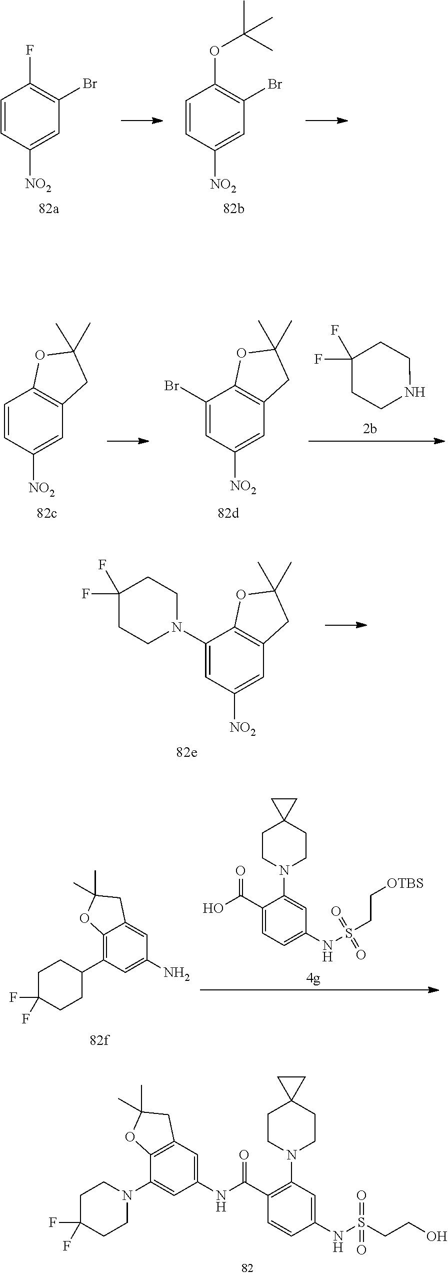

### Step 1: Synthesis of Compound 82b

Compound 82a (1.1 g, 5.0 mmol) was added to THE (10 mL), and then the mixture was cooled to 0 °C, followed by the addition of potassium tert-butoxide (678 mg, 6.0 mmol). The mixture was warmed to room temperature and reacted sufficiently. A saturated aqueous ammonium chloride solution (20 mL) was added to the system, and ethyl acetate (10 mL×3) was added for extraction. The combined organic phases were washed with saturated brine, dried over anhydrous magnesium sulfate, filtered, and concentrated to dryness by rotary evaporation, and the residue was purified by flash column chromatography (petroleum ether:ethyl acetate=12:1) to give compound 82b (1.25 g).

### Step 2: Synthesis of Compound 82c

Compound 82b (1.0 g, 3.6 mmol), palladium acetate (250 mg, 0.8 mmol), tricyclohexylphosphine (238 mg, 1.0 mmol), pivalic acid (360 mg, 3.6 mmol), and potassium carbonate (0.69 g, 5 mmol) were added to mesitylene (15 mL), and the mixture was reacted sufficiently at 140 °C in a nitrogen atmosphere. A saturated aqueous sodium bicarbonate solution (20 mL) was added to the system, and ethyl acetate (15 mL×3) was added for extraction. The combined organic phases were washed with saturated brine, dried over anhydrous magnesium sulfate, filtered, and concentrated to dryness by rotary evaporation, and the residue was purified by flash column chromatography (petroleum ether:ethyl acetate=10:1) to give compound 82c (570 mg).

### Step 3: Synthesis of Compound 82d

Compound 82c (385 mg, 2.0 mmol) was added to dioxane (5 mL), followed by the addition of bromine (480 mg, 3.0 mmol). The mixture was reacted sufficiently at room temperature. A saturated aqueous sodium bicarbonate solution (10 mL) was added to the system, and ethyl acetate (10 mL×3) was added for extraction. The combined organic phases were washed with saturated brine, dried over anhydrous magnesium sulfate, filtered, and concentrated to dryness by rotary evaporation, and the residue was purified by flash column chromatography (petroleum ether:ethyl acetate=10:1) to give compound 82d (401 mg).

### Step 4: Synthesis of Compound 82e

Compound 82e (199 mg) was obtained through synthesis and purification using a method identical to that described in step 4 of Example 22, with compound 82d (270 mg, 1.0 mmol) and compound 2b (132 mg, 1.1 mmol) as starting materials.

### Step 5: Synthesis of Compound 82f

Compound 82f (94 mg) was obtained through synthesis and purification using a method identical to that described in step 3 of Example 4, with compound 82e (190 mg, 0.61 mmol) as starting material.

MS m/z (ESI): 282.2 [M+H]⁺.

### Step 6: Synthesis of Compound 82

Compound 82 (12 mg) was obtained through synthesis and purification using a method identical to that described in step 5 of Example 23, with compound 82f (28 mg, 0.1 mmol) and compound 4g (47 mg, 0.1 mmol) as starting materials.

MS m/z (ESI): 619.3 [M+H]⁺.

### Example 83: Synthesis of Compound 83

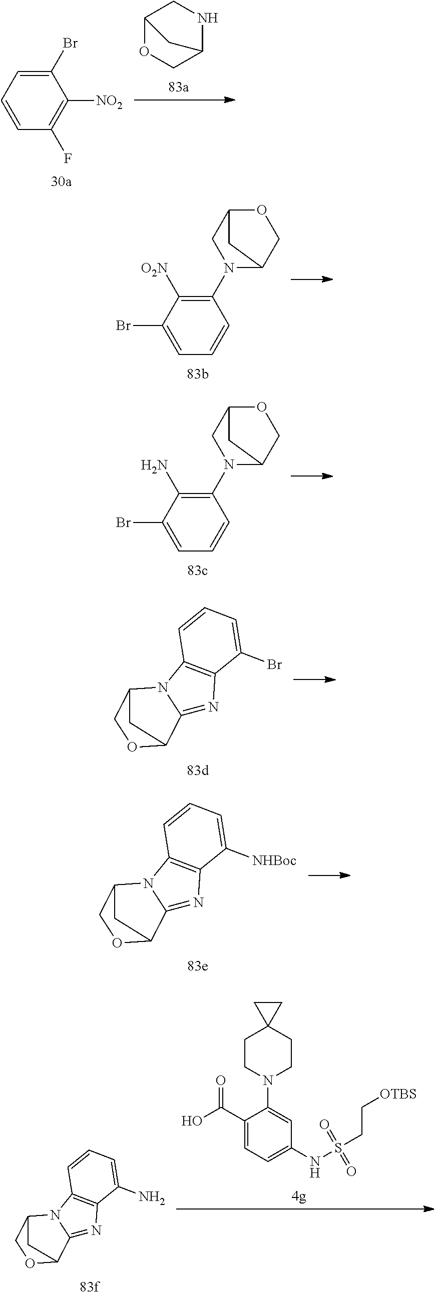

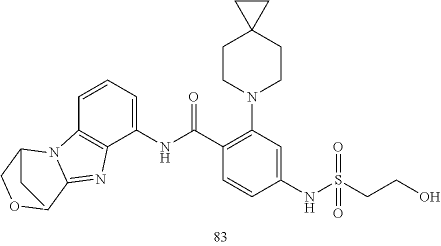

### Step 1: Synthesis of Compound 83b

Compound 83b (1.0 g) was obtained through synthesis and purification using a method identical to that described in step 1 of Example 30, with compound 30a (820 mg, 3.7 mmol) and 83a (hydrochloride, 500 mg, 3.7 mmol) as starting materials.

### Step 2: Synthesis of Compound 83c

Compound 83c (499 mg) was obtained through synthesis and purification using a method identical to that described in step 3 of Example 4, with compound 83b (1.0 g, 3.3 mmol) as starting material.

### Step 3: Synthesis of Compound 83d

Compound 83d (60 mg) was obtained through synthesis and purification using a method identical to that described in step 3 of Example 30, with compound 83c (200 mg, 0.74 mmol) as starting material.

### Step 4: Synthesis of Compound 83e

Compound 83e (17 mg) was obtained through synthesis and purification using a method identical to that described in step 4 of Example 30, with compound 83d (30 mg, 0.11 mmol) and tert-butyl carbamate (40 mg, 0.34 mmol) as starting materials.

### Step 5: Synthesis of Compound 83f

Compound 83f (21 mg) was obtained through synthesis and purification using a method identical to that described in step 5 of Example 30, with compound 83e (34 mg, 0.11 mmol) as starting material.

MS m/z (ESI): 202.1 [M+H]⁺.

### Step 6: Synthesis of Compound 83

Compound 83 (8 mg) was obtained through synthesis and purification using a method identical to that described in step 5 of Example 23, with compound 83f (20 mg, 0.1 mmol) and compound 4g (47 mg, 0.1 mmol) as starting materials.

MS m/z (ESI): 538.2 [M+H]⁺.

¹H NMR (400 MHz, DMSO-d₆) δ 12.39 (s, 1H), 10.14 (s, 1H), 8.37-8.30 (m, 1H), 8.02 (d, J=8.6 Hz, 1H), 7.36 (dd, J=8.1, 1.0 Hz, 1H), 7.27 (d, J=2.1 Hz, 1H), 7.22 (t, J=8.0 Hz, 1H), 7.08 (dd, J=8.6, 2.1 Hz, 1H), 5.66 (s, 1H), 5.44 (d, J=2.3 Hz, 1H), 4.93 (s, 1H), 3.98 (d, J=7.2 Hz, 1H), 3.76 (t, J=6.5 Hz, 2H), 3.36-3.35 (m, 2H), 3.01-2.96 (m, 5H), 2.46-2.42 (m, 1H), 2.36-2.32 (m, 1H), 1.88-1.69 (m, 4H), 0.31 (s, 4H).

### Example 84 and Example 85: Synthesis of Compound 84 and Compound 85

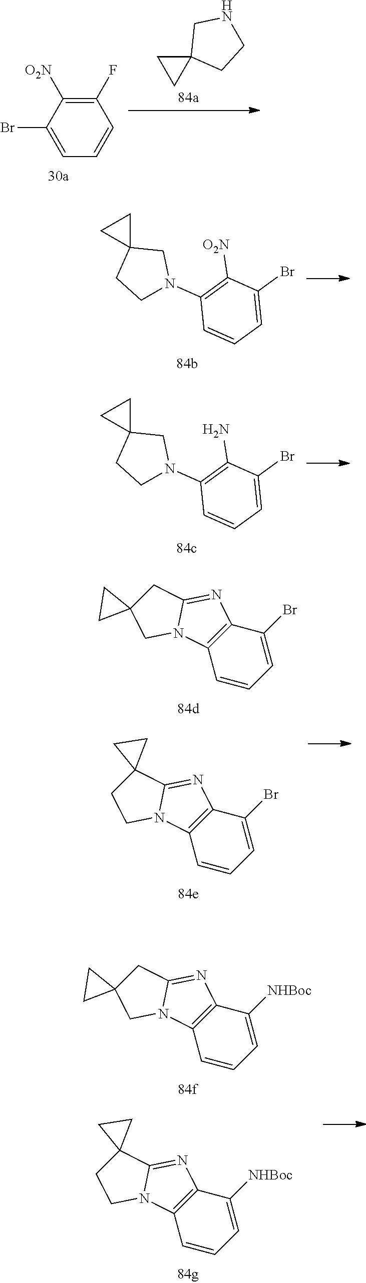

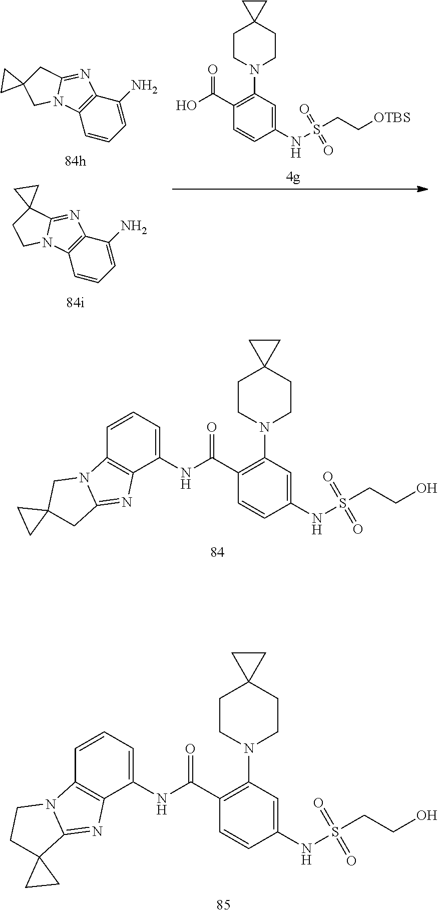 [SMILES: Nc1cccc2c1nc1n2CC2(CC2)C1 ; CC(C)(C)[Si](C)(C)OCCS(=O)(=O)Nc1ccc(C(=O)O)c(N2CCC3(CC2)CC3)c1 ; Nc1cccc2c1nc1n2CCC12CC2 ; O=C(Nc1cccc2c1nc1n2CC2(CC2)C1)c1ccc(NS(=O)(=O)CCO)cc1N1CCC2(CC1)CC2 ; O=C(Nc1cccc2c1nc1n2CCC12CC2)c1ccc(NS(=O)(=O)CCO)cc1N1CCC2(CC1)CC2]

### Step 1: Synthesis of Compound 84b

Compound 84b (1.13 g) was obtained through synthesis and purification using a method identical to that described in step 1 of Example 30, with compound 30a (1.00 g, 4.55 mmol) and 84a (hydrochloride, 0.73 g, 5.45 mmol) as starting materials.

### Step 2: Synthesis of Compound 84c

Compound 84c (0.91 g) was obtained through synthesis and purification using a method identical to that described in step 3 of Example 4, with compound 84b (1.13 g, 3.8 mmol) as starting material.

Step 3: Synthesis of Compound 84d and compound 84e

The mixture (0.7 g) of compound 84d and compound 84e was obtained through synthesis and purification using a method identical to that described in step 3 of Example 30, with compound 84c (0.91 g, 3.7 mmol) as starting material.

Step 4: Synthesis of Compound 84f and compound 84g

The mixture (0.3 g) of compound 84f and compound 84g was obtained through synthesis and purification using a method identical to that described in step 4 of Example 30, with the mixture (0.3 g, 1.14 mmol) of compound 84d and compound 84e and tert-butyl carbamate (0.2 g, 1.71 mmol) as starting materials.

Step 5: Synthesis of Compound 84 h and compound 84i

The mixture (182 mg) of compound 84 h and compound 84i was obtained through synthesis and purification using a method identical to that described in step 5 of Example 30, with the mixture (0.3 g, 1.0 mmol) of compound 84f and compound 84g as starting material.

MS m/z (ESI): 200.1 [M+H]⁺.

Step 6: Synthesis of Compound 84 and compound 85

The final products were obtained through synthesis and purification using a method identical to that described in step 5 of Example 23, with the mixture (170 mg, 0.85 mmol) of compound 84 h and compound 84i and compound 4g (398 mg, 0.85 mmol) as starting materials.

Compound 84 (10.6 mg)

MS m/z (ESI): 536.2 [M+H]⁺.

¹H NMR (400 MHz, DMSO-d₆) δ 12.38 (s, 1H), 8.35-8.30 (m, 1H), 8.01 (d, J=8.6 Hz, 1H), 7.24 (d, J=2.1 Hz, 1H), 7.16 (d, J=2.1 Hz, 1H), 7.15 (s, 1H), 7.09-7.04 (m, 1H), 4.11 (s, 2H), 3.76 (t, J=6.5 Hz, 2H), 3.34-3.32 (m, 2H), 3.02 (s, 2H), 3.01-2.93 (m, 4H), 1.98-1.61 (m, 4H), 0.88 (s, 4H), 0.29 (s, 4H).

Compound 85 (102 mg)

MS m/z (ESI): 536.2 [M+H]⁺.

¹H NMR (400 MHz, DMSO-d₆) δ 12.22 (s, 1H), 8.37 (dd, J=7.6, 1.5 Hz, 1H), 8.02 (d, J=8.6 Hz, 1H), 7.24 (s, 1H), 7.16 (dd, J=8.0, 1.4 Hz, 1H), 7.12 (t, J=7.8 Hz, 1H), 7.06 (d, J=8.5 Hz, 1H), 4.28 (t, J=7.2 Hz, 2H), 3.76 (t, J=6.5 Hz, 2H), 3.35-3.35 (m, 2H), 3.00 (d, J=15.8 Hz, 4H), 2.74 (t, J=7.2 Hz, 2H), 1.90-1.61 (m, 4H), 1.34-1.29 (m, 2H), 1.21-1.15 (m, 2H), 0.34 (s, 4H).

### Example 86: Synthesis of Compound 86

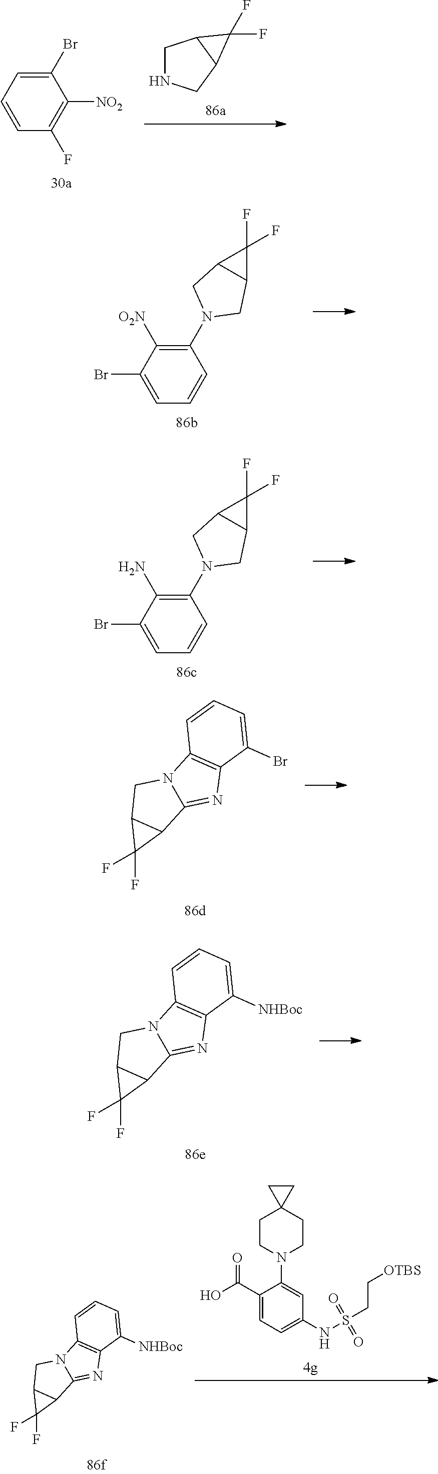

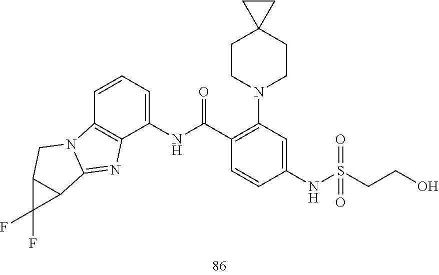 [SMILES: O=C(Nc1cccc2c1nc1n2CC2C1C2(F)F)c1ccc(NS(=O)(=O)CCO)cc1N1CCC2(CC1)CC2]

### Step 1: Synthesis of Compound 86b

Compound 86b (0.76 g) was obtained through synthesis and purification using a method identical to that described in step 1 of Example 30, with compound 30a (1.56 g, 7.07 mmol) and 86a (hydrochloride, 1.0 g, 6.43 mmol) as starting materials.

### Step 2: Synthesis of Compound 86c

Compound 86c (430 mg) was obtained through synthesis and purification using a method identical to that described in step 3 of Example 4, with compound 86b (710 mg, 2.22 mmol) as starting material.

### Step 3: Synthesis of Compound 86d

Compound 86d (140 mg) was obtained through synthesis and purification using a method identical to that described in step 3 of Example 30, with compound 86c (290 mg, 1.0 mmol) as starting material.

### Step 4: Synthesis of Compound 86e

Compound 86e (95 mg) was obtained through synthesis and purification using a method identical to that described in step 4 of Example 30, with compound 86d (100 mg, 0.35 mmol) and tert-butyl carbamate (164 mg, 1.4 mmol) as starting materials.

### Step 5: Synthesis of Compound 86f

Compound 86f (55 mg) was obtained through synthesis and purification using a method identical to that described in step 5 of Example 30, with compound 86e (90 mg, 0.28 mmol) as starting material.

MS m/z (ESI): 222.1 [M+H]⁺.

### Step 6: Synthesis of Compound 86

Compound 86 (7.9 mg) was obtained through synthesis and purification using a method identical to that described in step 5 of Example 23, with compound 86f (50 mg, 0.23 mmol) and compound 4g (104 mg, 0.23 mmol) as starting materials.

MS m/z (ESI): 558.1 [M+H]⁺.

¹H NMR (400 MHz, DMSO-d₆) δ 12.36 (s, 1H), 10.15 (s, 1H), 8.33 (dd, J=6.8, 2.1 Hz, 1H), 8.01 (d, J=8.6 Hz, 1H), 7.26 (d, J=2.1 Hz, 1H), 7.23-7.16 (m, 2H), 7.08 (dd, J=8.6, 2.0 Hz, 1H), 4.57 (dd, J=12.0, 6.6 Hz, 1H), 4.42 (d, J=11.9 Hz, 1H), 3.77 (t, J=6.5 Hz, 2H), 3.73 (d, J=9.2 Hz, 1H), 3.59-3.51 (m, 1H), 3.38-3.37 (m, 2H), 2.99 (t, J=5.4 Hz, 4H), 1.76 (s, 4H), 0.30 (s, 4H).

### Example 87: Synthesis of Compound 87

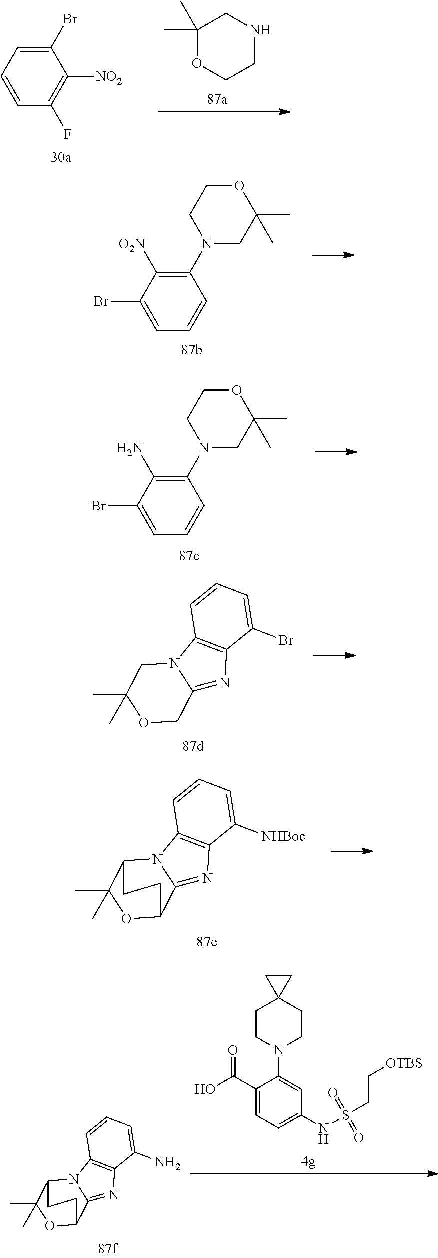

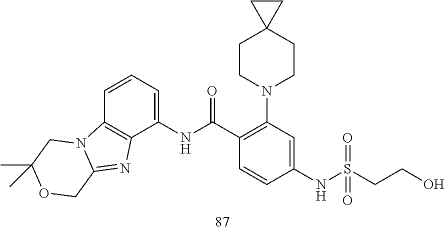 [SMILES: CC1(C)Cn2c(nc3c(NC(=O)c4ccc(NS(=O)(=O)CCO)cc4N4CCC5(CC4)CC5)cccc32)CO1]

### Step 1: Synthesis of Compound 87b

Compound 87b (0.4 g) was obtained through synthesis and purification using a method identical to that described in step 1 of Example 30, with compound 30a (0.3 g, 1.36 mmol) and 87a (172 mg, 1.49 mmol) as starting materials.

### Step 2: Synthesis of Compound 87c

Compound 87c (330 mg) was obtained through synthesis and purification using a method identical to that described in step 3 of Example 4, with compound 87b (0.4 g, 1.27 mmol) as starting material.

### Step 3: Synthesis of Compound 87d

Compound 87d (130 mg) was obtained through synthesis and purification using a method identical to that described in step 3 of Example 30, with compound 87c (330 mg, 1.16 mmol) as starting material.

### Step 4: Synthesis of Compound 87e

Compound 87e (95 mg) was obtained through synthesis and purification using a method identical to that described in step 4 of Example 30, with compound 87d (120 mg, 0.43 mmol) and tert-butyl carbamate (75 mg, 0.64 mmol) as starting materials.

### Step 5: Synthesis of Compound 87f

Compound 87f (60 mg) was obtained through synthesis and purification using a method identical to that described in step 5 of Example 30, with compound 87e (90 mg, 0.28 mmol) as starting material.

MS m/z (ESI): 218.1 [M+H]⁺.

### Step 6: Synthesis of Compound 87

Compound 87 (10.1 mg) was obtained through synthesis and purification using a method identical to that described in step 5 of Example 23, with compound 87f (46 mg, 0.21 mmol) and compound 4g (104 mg, 0.23 mmol) as starting materials.

MS m/z (ESI): 554.2 [M+H]⁺.

¹H NMR (400 MHz, DMSO-d₆) δ 12.52 (s, 1H), 8.42-8.34 (m, 1H), 8.04 (d, J=8.6 Hz, 1H), 7.26 (d, J=2.2 Hz, 1H), 7.23-7.18 (m, 2H), 7.08 (dd, J=8.6, 2.1 Hz, 1H), 4.98 (s, 2H), 4.10 (s, 2H), 3.77 (t, J=6.5 Hz, 2H), 3.34-3.32 (m, 2H), 2.97 (t, J=5.4 Hz, 4H), 2.01-1.53 (m, 4H), 1.37 (s, 6H), 0.31 (s, 4H).

### Example 88: Synthesis of Compound 88

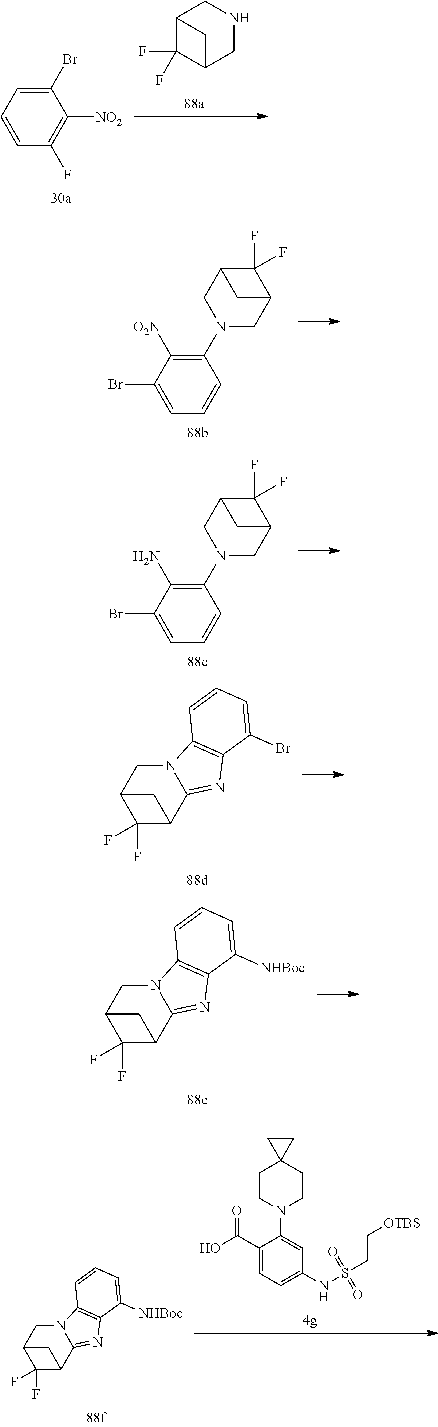

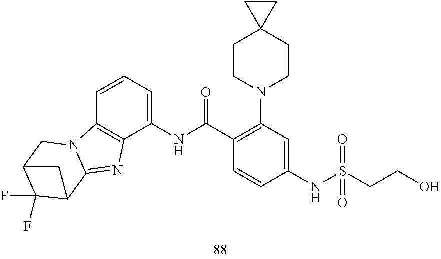 [SMILES: O=C(Nc1cccc2c1nc1n2CC2CC1C2(F)F)c1ccc(NS(=O)(=O)CCO)cc1N1CCC2(CC1)CC2]

### Step 1: Synthesis of Compound 88b

Compound 88b (0.2 g) was obtained through synthesis and purification using a method identical to that described in step 1 of Example 30, with compound 30a (824 mg, 3.75 mmol) and 88a (499 mg, 3.75 mmol) as starting materials.

### Step 2: Synthesis of Compound 88c

Compound 88c (80 mg) was obtained through synthesis and purification using a method identical to that described in step 3 of Example 4, with compound 88b (90 mg, 0.27 mmol) as starting material.

### Step 3: Synthesis of Compound 88d

Compound 88d (54 mg) was obtained through synthesis and purification using a method identical to that described in step 3 of Example 30, with compound 88c (76 mg, 0.25 mmol) as starting material.

### Step 4: Synthesis of Compound 88e

Compound 88e (46 mg) was obtained through synthesis and purification using a method identical to that described in step 4 of Example 30, with compound 88d (50 mg, 0.17 mmol) and tert-butyl carbamate (40 mg, 0.34 mmol) as starting materials.

### Step 5: Synthesis of Compound 88f

Compound 88f (30 mg) was obtained through synthesis and purification using a method identical to that described in step 5 of Example 30, with compound 88e (46 mg, 0.13 mmol) as starting material.

MS m/z (ESI): 236.1 [M+H]⁺.

### Step 6: Synthesis of Compound 88

Compound 88 (10.0 mg) was obtained through synthesis and purification using a method identical to that described in step 5 of Example 23, with compound 88f (30 mg, 0.13 mmol) and compound 4g (61 mg, 0.13 mmol) as starting materials.

MS m/z (ESI): 572.1 [M+H]⁺.

¹H NMR (400 MHz, DMSO-d₆) δ 12.49 (s, 1H), 8.38 (dd, J=6.3, 2.7 Hz, 1H), 8.02 (d, J=8.6 Hz, 1H), 7.26 (d, J=2.2 Hz, 1H), 7.25-7.20 (m, 2H), 7.07 (dd, J=8.7, 2.1 Hz, 1H), 4.41-4.34 (m, 1H), 4.32-4.24 (m, 2H), 3.76 (t, J=6.5 Hz, 2H), 3.61-3.53 (m, 1H), 3.35-3.34 (m, 2H), 3.14-3.07 (m, 1H), 3.05-2.93 (m, 4H), 2.80-2.72 (m, 1H), 1.89-1.62 (m, 4H), 0.28 (s, 4H).

### Example 89: Synthesis of Compound 89

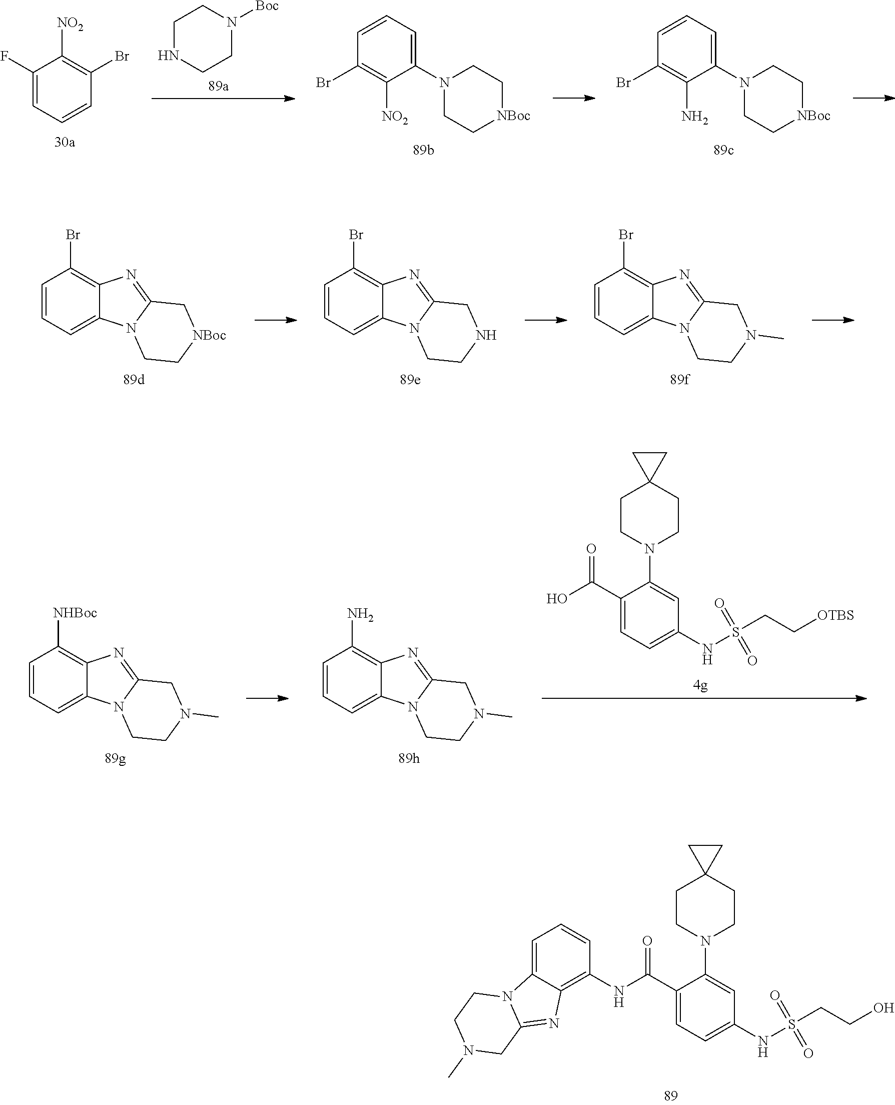

### Step 1: Synthesis of Compound 89b

Compound 89b (7.9 g) was obtained through synthesis and purification using a method identical to that described in step 1 of Example 30, with compound 30a (5.0 mg, 22.7 mmol) and 89a (5.08 g, 27.3 mmol) as starting materials.

### Step 2: Synthesis of Compound 89c

Compound 89c (7.1 g) was obtained through synthesis and purification using a method identical to that described in step 3 of Example 4, with compound 89b (7.9 g, 20.5 mmol) as starting material.

### Step 3: Synthesis of Compound 89d

Compound 89d (4.3 g) was obtained through synthesis and purification using a method identical to that described in step 3 of Example 30, with compound 89c (7.1 g, 20 mmol) as starting material.

### Step 4: Synthesis of Compound 89e

Compound 89e (370 mg) was obtained through synthesis and purification using a method identical to that described in step 5 of Example 30, with compound 89d (1.5 g, 4.26 mmol) as starting material.

### Step 5: Synthesis of Compound 89f

Compound 89e (360 mg, 1.43 mmol) and paraformaldehyde (471 mg, 1.43 mmol) were dissolved in methanol (2 mL), followed by the addition of acetic acid (0.1 mL). The mixture was stirred and reacted sufficiently at 50 °C. After the reaction mixture was cooled to room temperature, sodium cyanoborohydride (180 mg, 2.86 mmol) was added, and the mixture was stirred and reacted sufficiently at 50 °C. After the reaction mixture was cooled to room temperature, the volatiles were removed under vacuum, and the residue was purified by flash column chromatography (petroleum ether:ethyl acetate=80:20-66:34) to give compound 89f (0.3 g).

### Step 6: Synthesis of Compound 89g

Compound 89g (203 mg) was obtained through synthesis and purification using a method identical to that described in step 4 of Example 30, with compound 89f (300 mg, 1.13 mmol) and tert-butyl carbamate (198 mg, 1.69 mmol) as starting materials.

### Step 7: Synthesis of Compound 89h

Compound 89h (97 mg) was obtained through synthesis and purification using a method identical to that described in step 5 of Example 30, with compound 89g (198 mg, 0.66 mmol) as starting material.

MS m/z (ESI): 203.1 [M+H]⁺.

### Step 8: Synthesis of Compound 89

Compound 89 (10.6 mg) was obtained through synthesis and purification using a method identical to that described in step 5 of Example 23, with compound 89h (34.5 mg, 0.17 mmol) and compound 4g (80 mg, 0.17 mmol) as starting materials.

MS m/z (ESI): 539.2 [M+H]⁺.

¹H NMR (400 MHz, DMSO-d₆) δ 12.46 (s, 1H), 10.17 (s, 1H), 8.36 (d, J=7.0 Hz, 1H), 8.04 (d, J=8.6 Hz, 1H), 7.27 (s, 1H), 7.24-7.15 (m, 2H), 7.09 (d, J=8.6 Hz, 1H), 4.96 (s, 1H), 4.17 (s, 2H), 3.86-3.72 (m, 4H), 3.04-2.92 (m, 6H), 2.47 (s, 3H), 1.80 (s, 4H), 0.32 (s, 4H).

### Example 90: Synthesis of Compound 90

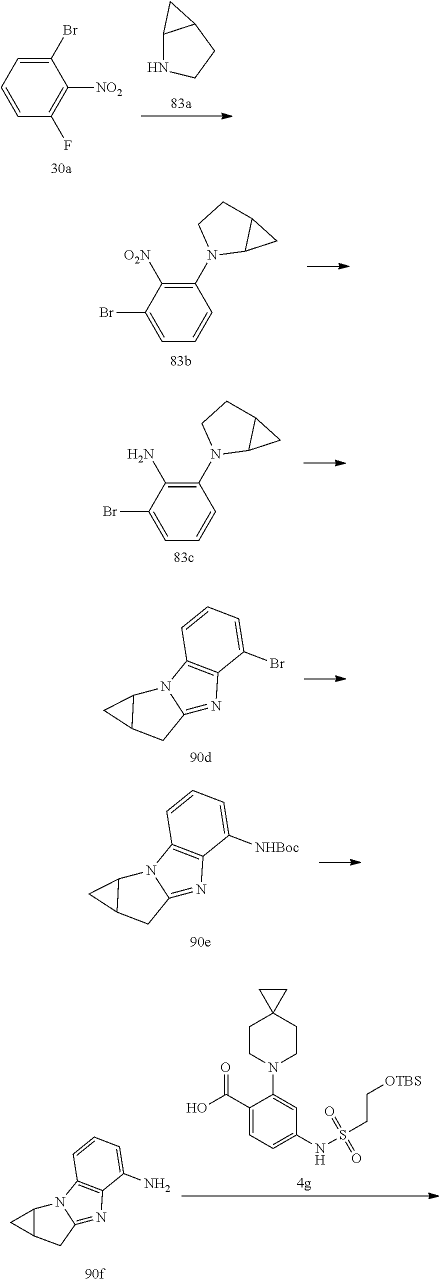

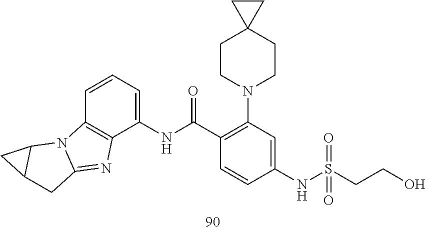 [SMILES: O=C(Nc1cccc2c1nc1n2C2CC2C1)c1ccc(NS(=O)(=O)CCO)cc1N1CCC2(CC1)CC2]

### Step 1: Synthesis of Compound 90b

Compound 90b (0.23 g) was obtained through synthesis and purification using a method identical to that described in step 1 of Example 30, with compound 30a (184 mg, 0.84 mmol) and 90a (hydrochloride, 100 mg, 0.84 mmol) as starting materials.

### Step 2: Synthesis of Compound 90c

Compound 90c (197 mg) was obtained through synthesis and purification using a method identical to that described in step 3 of Example 4, with compound 90b (0.23 g, 0.81 mmol) as starting material.

### Step 3: Synthesis of Compound 90d

Compound 90d (146 mg) was obtained through synthesis and purification using a method identical to that described in step 3 of Example 30, with compound 90c (190 mg, 0.75 mmol) as starting material.

### Step 4: Synthesis of Compound 90e

Compound 90e (65 mg) was obtained through synthesis and purification using a method identical to that described in step 4 of Example 30, with compound 90d (146 mg, 0.6 mmol) and tert-butyl carbamate (82 mg, 0.7 mmol) as starting materials.

### Step 5: Synthesis of Compound 90f

Compound 90f (37 mg) was obtained through synthesis and purification using a method identical to that described in step 5 of Example 30, with compound 90e (65 mg, 0.23 mmol) as starting material.

MS m/z (ESI): 186.1 [M+H]⁺.

### Step 6: Synthesis of Compound 90

Compound 90 (4.1 mg) was obtained through synthesis and purification using a method identical to that described in step 5 of Example 23, with compound 90f (17 mg, 0.1 mmol) and compound 4g (47 mg, 0.1 mmol) as starting materials.

MS m/z (ESI): 522.2 [M+H]⁺.

¹H NMR (400 MHz, DMSO-d₆) δ 12.49 (s, 1H), 8.43 (d, J=7.4 Hz, 1H), 8.38 (s, 1H), 8.03 (d, J=8.6 Hz, 1H), 7.33-7.15 (m, 3H), 7.05 (d, J=8.6 Hz, 1H), 5.99 (s, 1H), 5.43 (s, 1H), 4.28 (t, J=6.6 Hz, 2H), 3.76 (t, J=6.5 Hz, 2H), 3.31-3.29 (m, 4H), 3.02 (d, J=5.4 Hz, 4H), 1.94 (d, J=58.6 Hz, 4H), 0.31 (s, 4H).

### Example 91: Synthesis of Compound 91

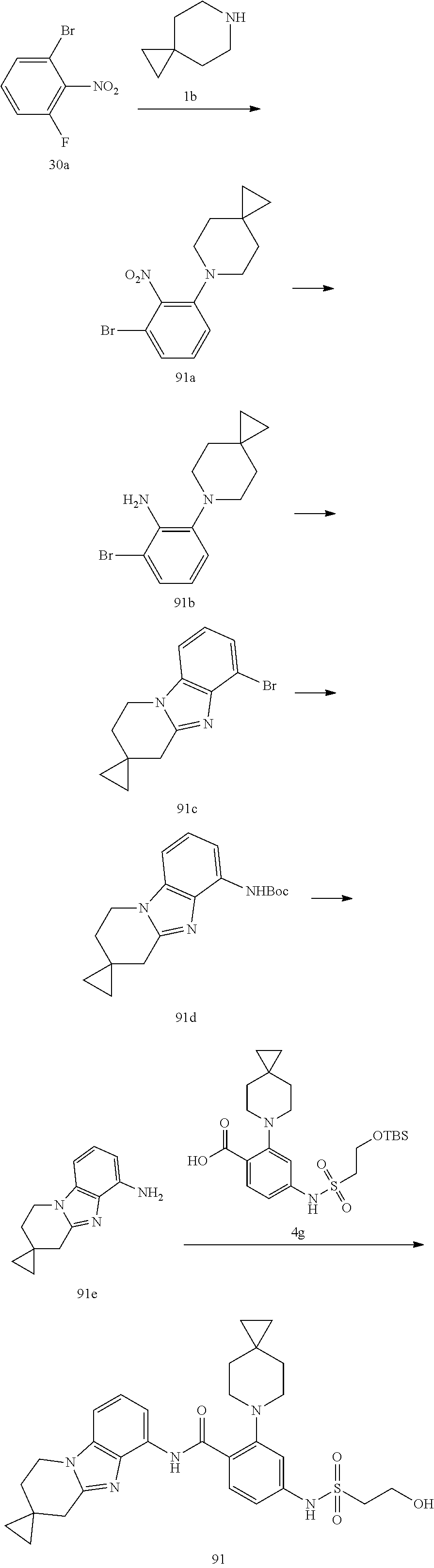

### Step 1: Synthesis of Compound 91a

Compound 91a (1.27 g) was obtained through synthesis and purification using a method identical to that described in step 1 of Example 30, with compound 30a (1.0 g, 4.55 mmol) and compound 1b (hydrochloride, 805 mg, 5.45 mmol) as starting materials.

### Step 2: Synthesis of Compound 91b

Compound 91b (790 mg) was obtained through synthesis and purification using a method identical to that described in step 3 of Example 4, with compound 91a (1.0 g, 3.2 mmol) as starting material.

### Step 3: Synthesis of Compound 91c

Compound 91c (465 mg) was obtained through synthesis and purification using a method identical to that described in step 3 of Example 30, with compound 91b (785 mg, 2.8 mmol) as starting material.

### Step 4: Synthesis of Compound 91d

Compound 91d (279 mg) was obtained through synthesis and purification using a method identical to that described in step 4 of Example 30, with compound 91c (400 mg, 1.44 mmol) and tert-butyl carbamate (334 mg, 2 mmol) as starting materials.

### Step 5: Synthesis of Compound 91e

Compound 91e (127 mg) was obtained through synthesis and purification using a method identical to that described in step 5 of Example 30, with compound 91d (279 mg, 0.89 mmol) as starting material.

MS m/z (ESI): 214.1 [M+H]⁺.

### Step 6: Synthesis of Compound 91

Compound 91 (7.4 mg) was obtained through synthesis and purification using a method identical to that described in step 5 of Example 23, with compound 91e (21 mg, 0.1 mmol) and compound 4g (47 mg, 0.1 mmol) as starting materials.

MS m/z (ESI): 550.1 [M+H]⁺.

¹H NMR (400 MHz, DMSO-d₆) δ 12.41 (s, 1H), 10.14 (s, 1H), 8.34 (dd, J=7.5, 1.3 Hz, 1H), 8.03 (d, J=8.6 Hz, 1H), 7.25 (d, J=2.1 Hz, 1H), 7.23-7.19 (m, 1H), 7.17 (t, J=7.8 Hz, 1H), 7.08 (dd, J=8.6, 2.1 Hz, 1H), 4.93 (t, J=5.7 Hz, 1H), 4.17 (t, J=6.0 Hz, 2H), 3.81-3.72 (m, 2H), 3.37-3.34 (m, 2H), 3.02-2.93 (m, 4H), 2.90 (s, 2H), 1.95 (t, J=6.0 Hz, 2H), 1.91-1.53 (m, 4H), 0.64-0.59 (m, 2H), 0.53-0.48 (m, 2H), 0.28 (s, 4H).

### Example 92: Synthesis of Compound 92

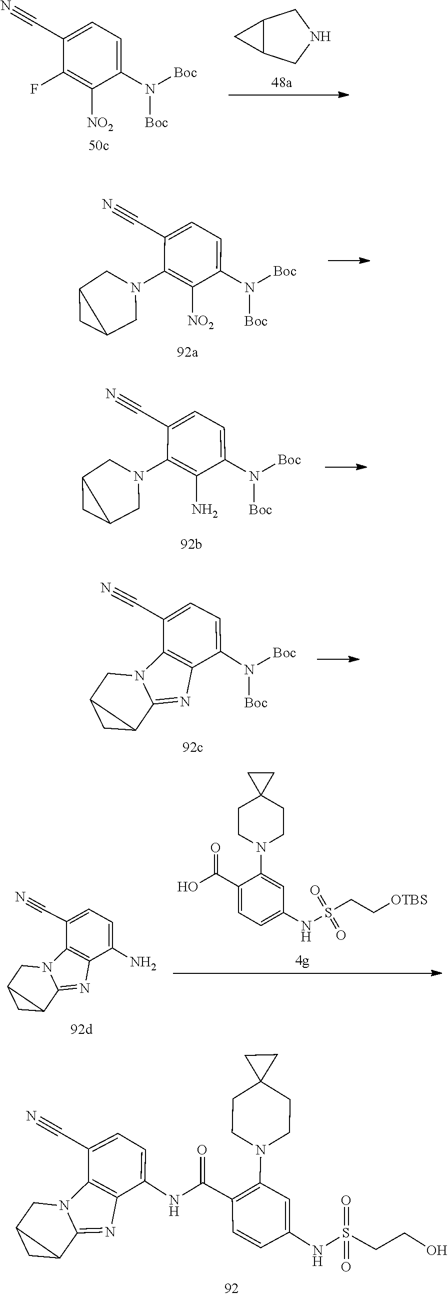

### Step 1: Synthesis of Compound 92a

Compound 92a (129 mg) was obtained through synthesis and purification using a method identical to that described in step 1 of Example 30, with compound 50c (121 mg, 0.31 mmol) and compound 48a (hydrochloride, 60 mg, 0.5 mmol) as starting materials.

### Step 3: Synthesis of Compound 92b

Compound 92b (63 mg) was obtained through synthesis and purification using a method identical to that described in step 3 of Example 4, with compound 92a (120 mg, 0.27 mmol) as starting material.

### Step 4: Synthesis of Compound 92c

Compound 92c (45 mg) was obtained through synthesis and purification using a method identical to that described in step 3 of Example 30, with compound 92b (63 mg, 0.15 mmol) as starting material.

### Step 5: Synthesis of Compound 92d

Compound 92c (45 mg, 0.11 mmol) was added to anhydrous dioxane (1 mL), followed by the addition of a solution of hydrochloric acid in dioxane (4 N, 0.2 mL). The mixture was reacted sufficiently at room temperature. The reaction mixture was filtered, and the filter cake was dried to give compound 92d (21 mg).

MS m/z (ESI): 211.1 [M+H]⁺.

### Step 6: Synthesis of Compound 92

Compound 92 (5.0 mg) was obtained through synthesis and purification using a method identical to that described in step 5 of Example 23, with compound 4g (47 mg, 0.1 mmol) and 92d (21 mg, 0.1 mmol) as starting materials.

MS m/z (ESI): 547.3 [M+H]⁺.

### Example 93: Synthesis of Compound 93

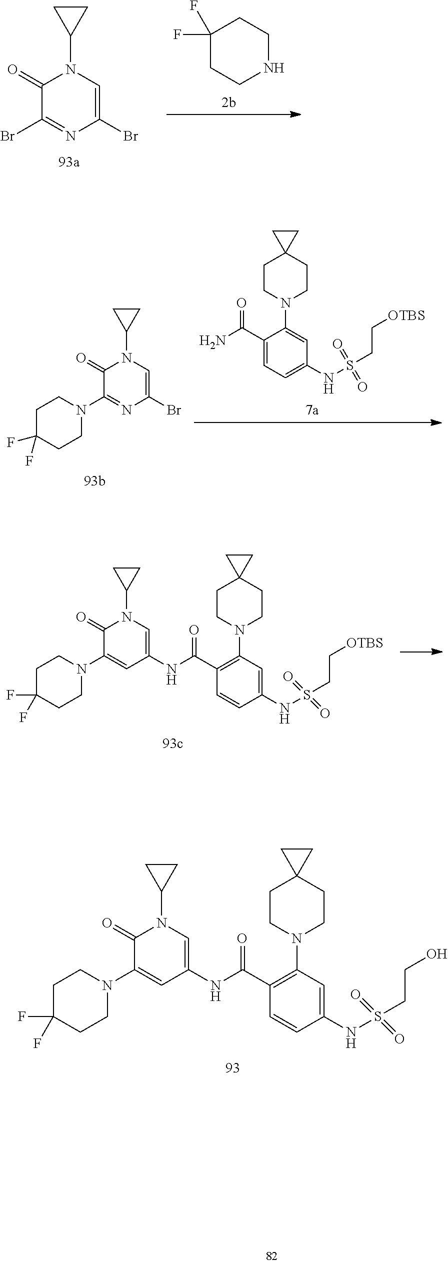 [SMILES: O=c1c(Br)nc(Br)cn1C1CC1 ; FC1(F)CCNCC1 ; O=c1c(N2CCC(F)(F)CC2)nc(Br)cn1C1CC1 ; CC(C)(C)[Si](C)(C)OCCS(=O)(=O)Nc1ccc(C(N)=O)c(N2CCC3(CC2)CC3)c1 ; CC(C)(C)[Si](C)(C)OCCS(=O)(=O)Nc1ccc(C(=O)Nc2cc(N3CCC(F)(F)CC3)c(=O)n(C3CC3)c2)c(N2CCC3(CC2)CC3)c1 ; O=C(Nc1cc(N2CCC(F)(F)CC2)c(=O)n(C2CC2)c1)c1ccc(NS(=O)(=O)CCO)cc1N1CCC2(CC1)CC2]

### Step 1: Synthesis of Compound 93b

Compound 93b (54.1 mg) was obtained through synthesis and purification using a method identical to that described in step 1 of Example 30, with compound 93a (64 mg, 0.22 mmol) and compound 2b (266 mg, 2.2 mmol) as starting materials.

### Step 2: Synthesis of Compound 93c

Compound 93c (22 mg) was obtained through synthesis and purification using a method identical to that described in step 2 of Example 7, with compound 7a (16 mg, 0.05 mmol) and 93b (16 mg, 0.05 mmol) as starting materials.

### Step 3: Synthesis of Compound 93

Compound 93 (13.7 mg) was obtained through synthesis and purification using a method identical to that described in step 8 of Example 4, with compound 93c (22 mg, 30 μmol) as starting material.

MS m/z (ESI): 607.3 [M+H]⁺.

### Example 95: Synthesis of Compound 95

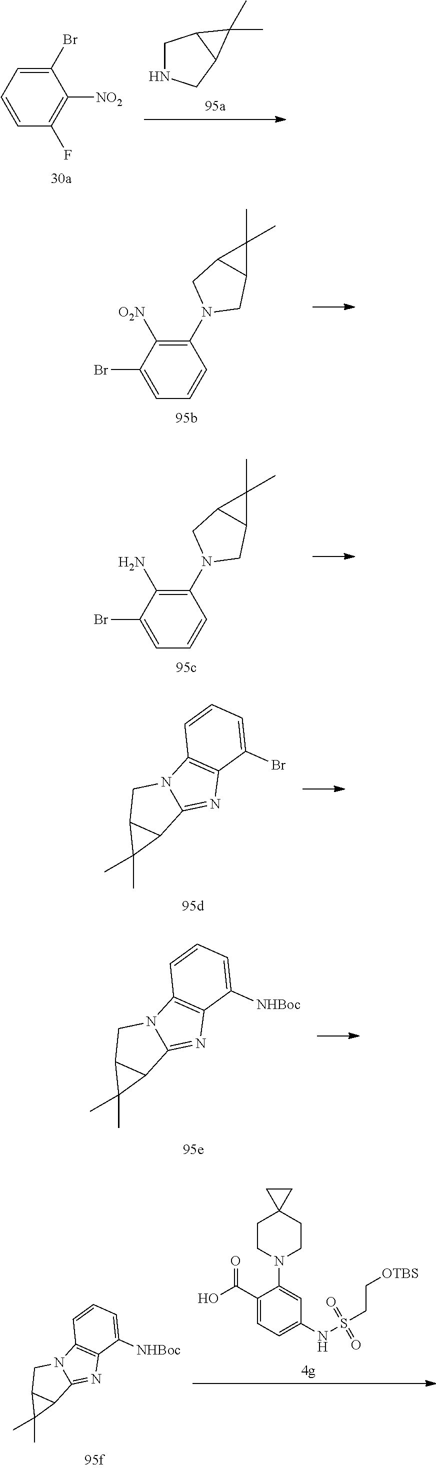

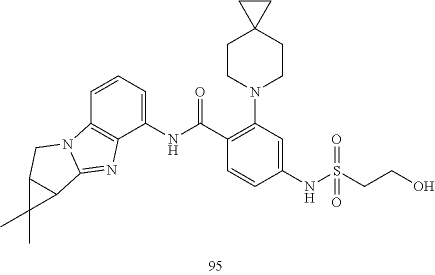 [SMILES: CC1(C)C2Cn3c(nc4c(NC(=O)c5ccc(NS(=O)(=O)CCO)cc5N5CCC6(CC5)CC6)cccc43)C21]

### Step 1: Synthesis of Compound 95b

Compound 95b (8.9 g) was obtained through synthesis and purification using a method identical to that described in step 1 of Example 30, with compound 30a (6.50 g, 29.6 mmol) and 95a (3.61 g, 32.5 mmol) as starting materials.

### Step 2: Synthesis of Compound 95c

Compound 95c (5.9 g) was obtained through synthesis and purification using a method identical to that described in step 3 of Example 4, with compound 95b (8.9 g, 28.6 mmol) as starting material.

### Step 3: Synthesis of Compound 95d

Compound 95d (3.4 g) was obtained through synthesis and purification using a method identical to that described in step 3 of Example 30, with compound 95c (5.7 g, 1.76 mmol) as starting material.

### Step 4: Synthesis of Compound 95e

Compound 95e (280 mg) was obtained through synthesis and purification using a method identical to that described in step 4 of Example 30, with compound 95d (300 mg, 1.1 mmol) and tert-butyl carbamate (507 mg, 4.3 mmol) as starting materials.

### Step 5: Synthesis of Compound 95f

Compound 95f (130 mg) was obtained through synthesis and purification using a method identical to that described in step 5 of Example 30, with compound 95e (280 mg, 0.89 mmol) as starting material.

MS m/z (ESI): 214.1 [M+H]⁺.

### Step 6: Synthesis of Compound 95

Compound 95 (30 mg) was obtained through synthesis and purification using a method identical to that described in step 5 of Example 23, with compound 95f (92 mg, 0.43 mmol) and compound 4g (100 mg, 0.21 mmol) as starting materials.

MS m/z (ESI): 550.1 [M+H]⁺.

¹H NMR (400 MHz, DMSO-d₆) δ 12.32 (s, 1H), 9.80 (s, 1H), 8.26 (dd, J=7.2, 1.7 Hz, 1H), 7.99 (d, J=8.6 Hz, 1H), 7.25 (d, J=2.1 Hz, 1H), 7.13-7.04 (m, 3H), 5.03 (s, 1H), 4.31 (dd, J=11.8, 5.3 Hz, 1H), 3.98 (d, J=11.8 Hz, 1H), 3.76 (t, J=6.5 Hz, 2H), 3.34 (t, J=6.6 Hz, 2H), 3.05-2.92 (m, 4H), 2.41-2.34 (m, 2H), 1.98-1.53 (m, 4H), 1.22 (s, 3H), 0.64 (s, 3H), 0.30 (s, 4H).

### Example 96: Synthesis of Compound 96

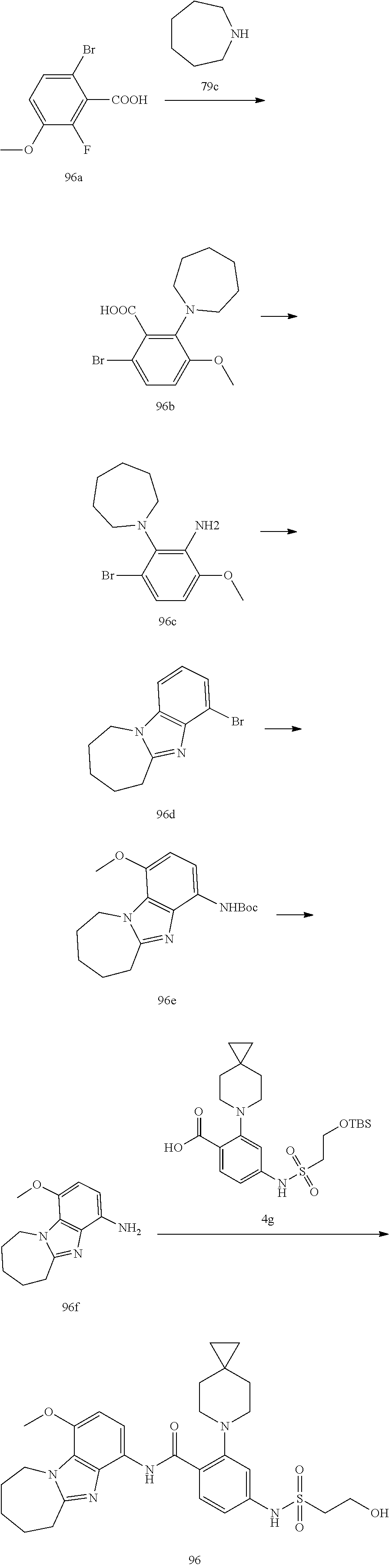

### Step 1: Synthesis of Compound 96b

Compound 96a (1.00 g, 4.02 mmol) was added to compound 79c (5 mL), and the mixture was reacted sufficiently at 100 °C. After the reaction mixture was cooled, the volatiles were removed under vacuum, and the residue was purified by flash column chromatography (dichloromethane:methanol=100:0-95:5) to give compound 96b (400 mg).

### Step 2: Synthesis of Compound 96c

Compound 96b (295 mg, 0.91 mmol), triethylamine (185 mg, 1.83 mmol), and diphenylphosphoryl azide (1.11 g, 4.57 mmol) were added to tert-butanol (5 mL) and water (1 mL), and the mixture was reacted sufficiently at 100 °C. After being cooled, the reaction mixture was poured into water and extracted with ethyl acetate (60 mL×3). The combined organic phases were dried over anhydrous sodium sulfate, filtered, and concentrated to dryness by rotary evaporation, and the residue was purified by flash column chromatography (petroleum ether:ethyl acetate=100:0-90:10) to give compound 96c (60.0 mg).

### Step 3: Synthesis of Compound 96d

Compound 96d (43 mg) was obtained through synthesis and purification using a method identical to that described in step 3 of Example 30, with compound 96c (60 mg, 0.15 mmol) as starting material.

### Step 4: Synthesis of Compound 96e

Compound 96e (24 mg) was obtained through synthesis and purification using a method identical to that described in step 4 of Example 30, with compound 96d (25 mg, 0.085 mmol) and tert-butyl carbamate (40 mg, 0.34 mmol) as starting materials.

### Step 5: Synthesis of Compound 96f

Compound 96f (13 mg) was obtained through synthesis and purification using a method identical to that described in step 5 of Example 30, with compound 96e (20 mg, 0.06 mmol) as starting material.

MS m/z (ESI): 231.1 [M+H]⁺.

### Step 6: Synthesis of Compound 96

Compound 96 (1.1 mg) was obtained through synthesis and purification using a method identical to that described in step 5 of Example 23, with compound 96f (13 mg, 0.056 mmol) and compound 4g (26 mg, 0.056 mmol) as starting materials.

MS m/z (ESI): 568.1 [M+H]⁺.

¹H NMR (400 MHz, DMSO-d₆) 12.29 (s, 1H), 10.10 (s, 1H), 8.20 (d, J=8.6 Hz, 1H), 8.03 (d, J=8.4 Hz, 1H), 7.26 (d, J=2.1 Hz, 1H), 7.08 (dd, J=8.6, 2.1 Hz, 1H), 6.71 (d, J=8.7 Hz, 1H), 4.92 (t, J=5.6 Hz, 1H), 4.68-4.60 (m, 2H), 3.89 (s, 3H), 3.78-3.72 (m, 2H), 3.38-3.37 (m, 2H), 3.10-3.05 (m, 2H), 3.00-2.94 (m, 4H), 1.95-1.79 (m, 6H), 1.78-1.74 (m, 2H), 1.71-1.66 (m, 2H), 0.30 (s, 4H).

### Example 100: Synthesis of Compound 100

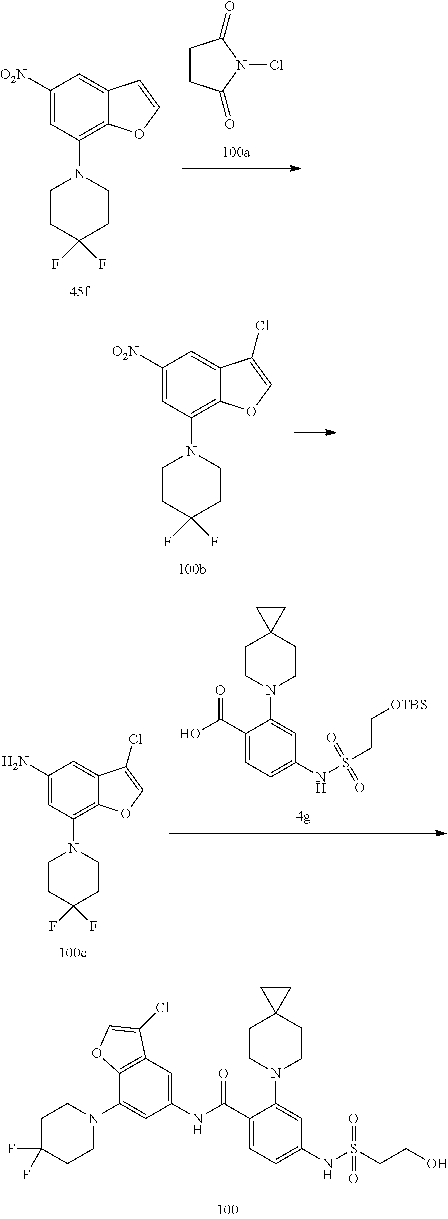

### Step 1: Synthesis of Compound 100b

Compound 45f (60 mg, 0.21 mmol) was dissolved in dichloromethane (2 mL) and trifluoroacetic acid (2 mL), followed by the addition of compound 100a (43 mg, 0.32 mmol). The mixture was stirred and reacted sufficiently at 50 °C. A saturated sodium bicarbonate solution (20 mL) was added, and dichloromethane (3×20 mL) was added for extraction. The combined organic phases were dried over anhydrous sodium sulfate, filtered, and concentrated, and the residue was purified by flash column chromatography to give 100b (30.0 mg).

### Step 2: Synthesis of Compound 100c

Compound 100c (21 mg) was obtained through synthesis and purification using a method identical to that described in step 3 of Example 4, with compound 100b (25 mg, 0.79 mmol) as starting material.

MS m/z (ESI): 287.0 [M+H]⁺.

### Step 3: Synthesis of Compound 100

Compound 100 (12 mg) was obtained through synthesis and purification using a method identical to that described in step 5 of Example 23, with compound 100c (15 mg, 0.52 mmol) and compound 4g (24.5 mg, 0.52 mmol) as starting materials.

MS m/z (ESI): 623.1 [M+H]⁺.

¹H NMR (400 MHz, DMSO-d₆) δ 11.52 (s, 1H), 8.28 (s, 1H), 8.06 (d, J=2.1 Hz, 1H), 7.93 (d, J=8.5 Hz, 1H), 7.23 (d, J=2.1 Hz, 1H), 7.06 (dd, J=8.5, 2.1 Hz, 1H), 7.02 (d, J=2.2 Hz, 1H), 3.77 (t, J=6.5 Hz, 2H), 3.40 (t, J=5.6 Hz, 4H), 3.36-3.34 (m, 2H), 3.02 (t, J=5.3 Hz, 4H), 2.23-2.10 (m, 4H), 1.51 (s, 4H), 0.33 (s, 4H).

### Example 101: Synthesis of Compound 101

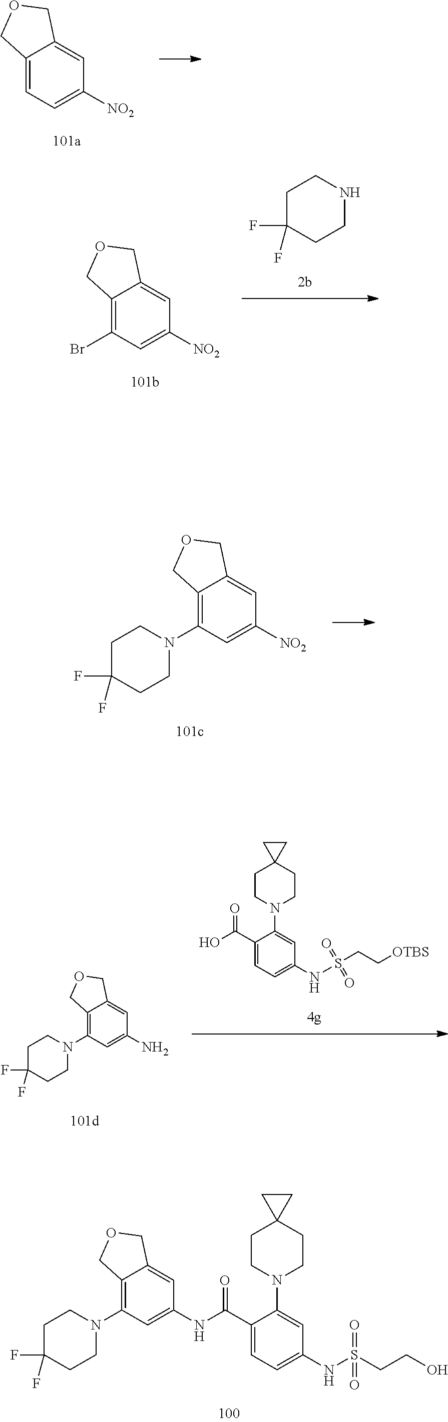

Step 1: synthesis of compound 101b

Compound 101b (550 mg) was obtained through synthesis and purification using a method identical to that described in step 1 of Example 41, with compound 101a (800 mg, 4.84 mmol) as starting material.

### Step 2: Synthesis of Compound 101c

Compound 101c (250 mg) was obtained through synthesis and purification using a method identical to that described in step 4 of Example 22, with compound 101b (300 mg, 1.23 mmol) and compound 2b (hydrochloride, 213 mg, 1.35 mmol) as starting materials.

### Step 3: Synthesis of Compound 101d

Compound 101d (70 mg) was obtained through synthesis and purification using a method identical to that described in step 3 of Example 4, with compound 101c (100 mg, 0.35 mmol) as starting material.

MS m/z (ESI): 255.1 [M+H]⁺.

### Step 4: Synthesis of Compound 101

Compound 101 (15.0 mg) was obtained through synthesis and purification using a method identical to that described in step 5 of Example 23, with compound 101d (23 mg, 0.09 mmol) and compound 4g (30 mg, 0.064 mmol) as starting materials.

MS m/z (ESI): 591.1 [M+H]⁺.

¹H NMR (400 MHz, DMSO-d₆) δ 11.71 (s, 1H), 7.82 (d, J=8.5 Hz, 1H), 7.51 (d, J=1.5 Hz, 1H), 7.16 (d, J=2.1 Hz, 1H), 7.13 (d, J=1.7 Hz, 1H), 7.03 (dd, J=8.5, 2.1 Hz, 1H), 5.01-4.97 (m, 2H), 4.97-4.94 (m, 2H), 3.76 (t, J=6.6 Hz, 2H), 3.34-3.33 (m, 2H), 3.16-3.08 (m, 4H), 3.01-2.93 (m, 4H), 2.15-2.03 (m, 4H), 1.60-1.49 (m, 4H), 0.35 (s, 4H).

### Example 103: Synthesis of Compound 103

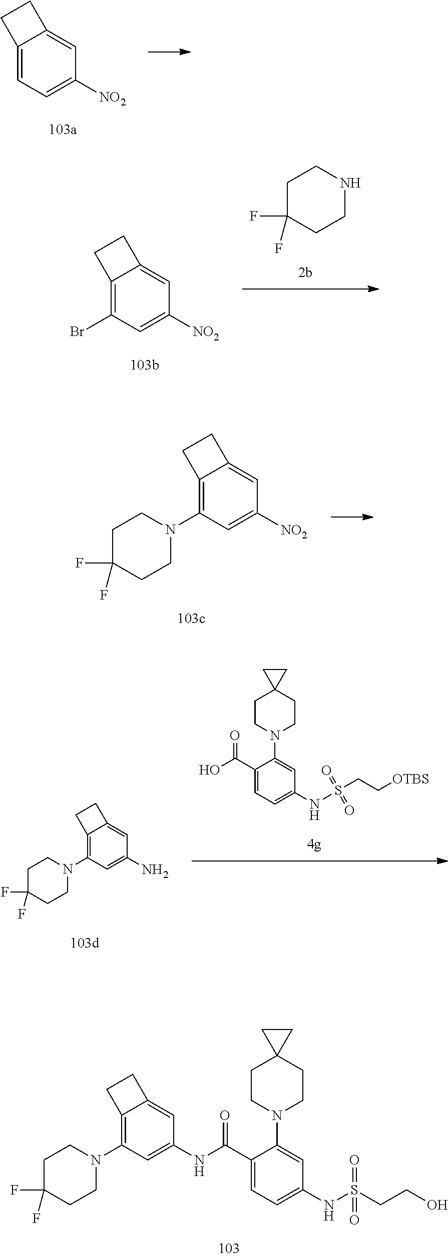

### Step 1: Synthesis of Compound 103b

Compound 103b (610 mg) was obtained through synthesis and purification using a method identical to that described in step 1 of Example 41, with compound 103a (1.0 g, 6.7 mmol) as starting material.

### Step 2: Synthesis of Compound 103c

Compound 103c (145 mg) was obtained through synthesis and purification using a method identical to that described in step 4 of Example 22, with compound 103b (600 mg, 2.63 mmol) and compound 2b (hydrochloride, 498 mg, 3.16 mmol) as starting materials.

### Step 3: Synthesis of Compound 103d

Compound 103d (71 mg) was obtained through synthesis and purification using a method identical to that described in step 3 of Example 4, with compound 103c (100 mg, 0.37 mmol) as starting material.

MS m/z (ESI): 239.1 [M+H]⁺.

### Step 4: Synthesis of Compound 103

Compound 103 (5.0 mg) was obtained through synthesis and purification using a method identical to that described in step 5 of Example 23, with compound 103d (10 mg, 0.042 mmol) and compound 4g (19.6 mg, 0.042 mmol) as starting materials.

MS m/z (ESI): 575.1 [M+H]⁺.

¹H NMR (400 MHz, DMSO-d₆) δ 11.54 (s, 1H), 10.08 (s, 1H), 7.84 (d, J=8.5 Hz, 1H), 7.20-7.13 (m, 2H), 7.04 (dd, J=8.5, 2.1 Hz, 1H), 6.94 (s, 1H), 3.76 (t, J=6.5 Hz, 2H), 3.41-3.40 (m, 2H), 3.33-3.31 (m, 4H), 3.22-3.18 (m, 2H), 3.07-3.02 (m, 2H), 2.99-2.93 (m, 4H), 2.08-1.98 (m, 4H), 1.60-1.51 (m, 4H), 0.35 (s, 4H).

### Example 105: Synthesis of Compound 105

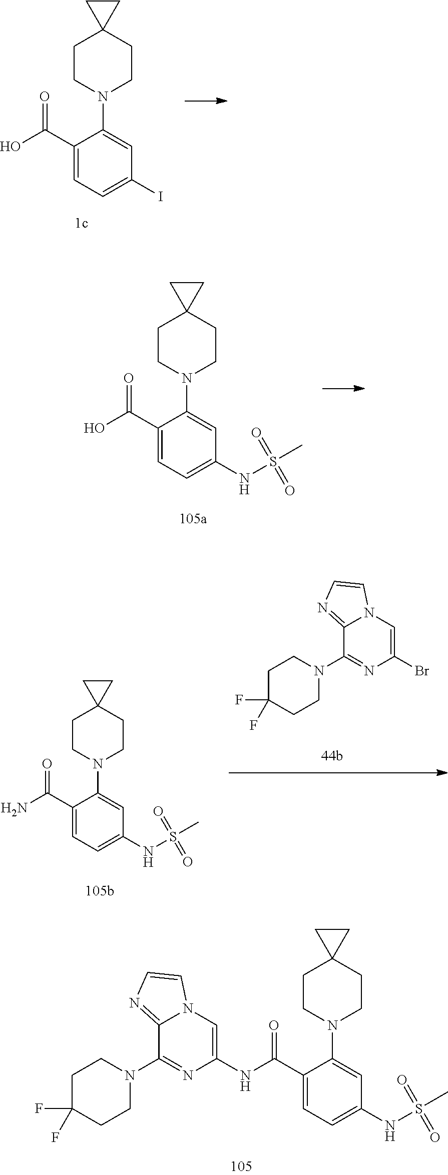 [SMILES: O=C(O)c1ccc(I)cc1N1CCC2(CC1)CC2 ; CS(=O)(=O)Nc1ccc(C(=O)O)c(N2CCC3(CC2)CC3)c1 ; CS(=O)(=O)Nc1ccc(C(N)=O)c(N2CCC3(CC2)CC3)c1 ; FC1(F)CCN(c2nc(Br)cn3ccnc23)CC1 ; CS(=O)(=O)Nc1ccc(C(=O)Nc2cn3ccnc3c(N3CCC(F)(F)CC3)n2)c(N2CCC3(CC2)CC3)c1]

### Step 1: Synthesis of Compound 105a

Compound 105a (485 mg) was obtained through synthesis and purification using a method identical to that described in step 3 of Example 1, with compound 1c (1.0 g, 2.8 mmol) and methanesulfonamide (0.54 g, 5.6 mmol) as starting materials.

### Step 2: Synthesis of Compound 105b

Compound 105b (135 mg) was obtained through synthesis and purification using a method identical to that described in step 1 of Example 7, with compound 105a (0.32 g, 1.0 mmol) as starting material.

### Step 3: Synthesis of Compound 105

Compound 105 (18 mg) was obtained through synthesis and purification using a method identical to that described in step 2 of Example 7, with compound 105b (94 mg, 0.29 mmol) and compound 44b (138 mg, 0.44 mmol) as starting materials.

MS m/z (ESI): 560.2 [M+H]⁺.

¹H NMR (400 MHz, DMSO-d₆) δ 12.88 (s, 1H), 10.22 (s, 1H), 8.84 (s, 1H), 8.12-8.06 (m, 2H), 7.55 (d, J=1.1 Hz, 1H), 7.27 (d, J=2.1 Hz, 1H), 7.14 (dd, J=8.6, 2.1 Hz, 1H), 4.51-4.35 (m, 4H), 3.12 (s, 3H), 3.03-2.95 (m, 4H), 2.16-2.05 (m, 4H), 1.86-1.62 (m, 4H), 0.41 (s, 4H).

### Example 106 Synthesis of Compound 106

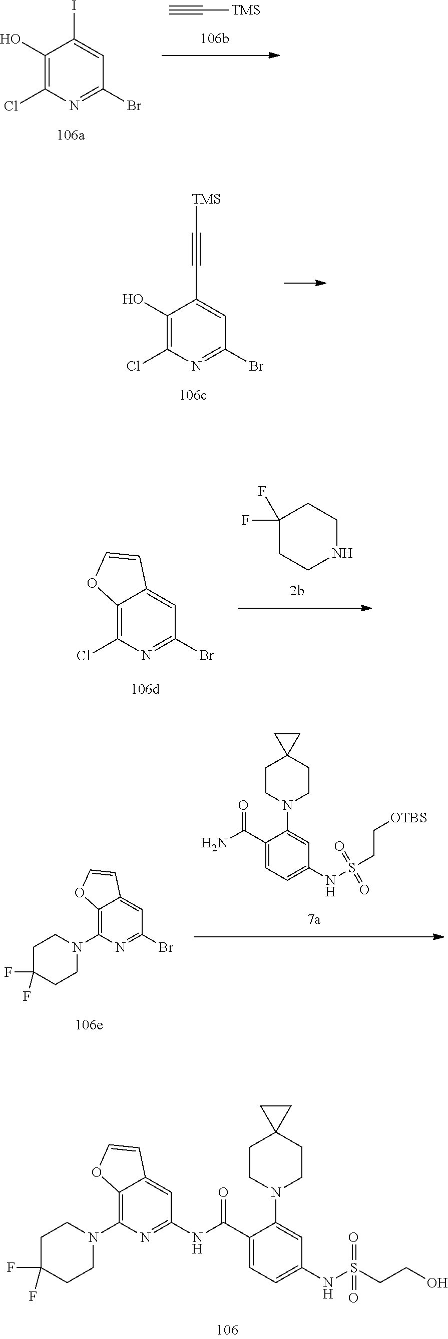

### Step 1: Synthesis of Compound 106c

Compound 106a (2.0 g, 6.0 mmol), compound 106b (1.02 mL, 7.2 mmol), cuprous iodide (110 mg, 0.6 mmol), and Pd(Ph₃P)₄ (420 mg, 0.6 mmol) were added to tetrahydrofuran (20 mL), followed by the addition of triethylamine (1.3 mL, 9.0 mmol) under an ice bath. The mixture was reacted sufficiently at room temperature. The volatiles were removed under vacuum, and the residue was purified by flash column chromatography to give compound 106c (1.54 g).

### Step 2: Synthesis of Compound 106d

Compound 106c (1.5 g, 5.0 mmol) was added to ethanol (2 mL), followed by the addition of cuprous iodide (92 mg, 0.5 mmol) and triethylamine (2 mL). The mixture was reacted sufficiently at 70 °C. The volatiles were removed under vacuum, and the residue was purified by flash column chromatography to give compound 106d (0.87 g).

### Step 3: Synthesis of Compound 106e

Compound 106e (1.5 g) was obtained through synthesis and purification using a method identical to that described in step 1 of Example 96, with compound 106d (0.8 g, 0.35 mmol) and compound 2b (2 mL) as starting materials.

### Step 4: Synthesis of Compound 106

Compound 106 (13 mg) was obtained through synthesis and purification using a method identical to that described in step 2 of Example 7, with compound 106e (63 mg, 0.2 mmol) and compound 7a (103 mg, 0.22 mmol) as starting materials.

MS m/z (ESI): 590.2 [M+H]⁺.

¹H NMR (400 MHz, DMSO-d₆) δ 12.81 (s, 1H), 10.17 (br, 1H), 8.09 (d, J=6.9 Hz, 1H), 8.07 (s, 1H), 7.27 (d, J=2.2 Hz, 1H), 7.13 (dd, J=8.6, 2.1 Hz, 1H), 6.99 (d, J=2.1 Hz, 1H), 3.97 (t, J=5.5 Hz, 2H), 3.76 (t, J=6.5 Hz, 4H), 2.99 (t, J=5.4 Hz, 4H), 2.19-2.05 (m, 4H), 1.76 (br, 4H), 0.39 (s, 4H).

### Example 107: Synthesis of Compound 107

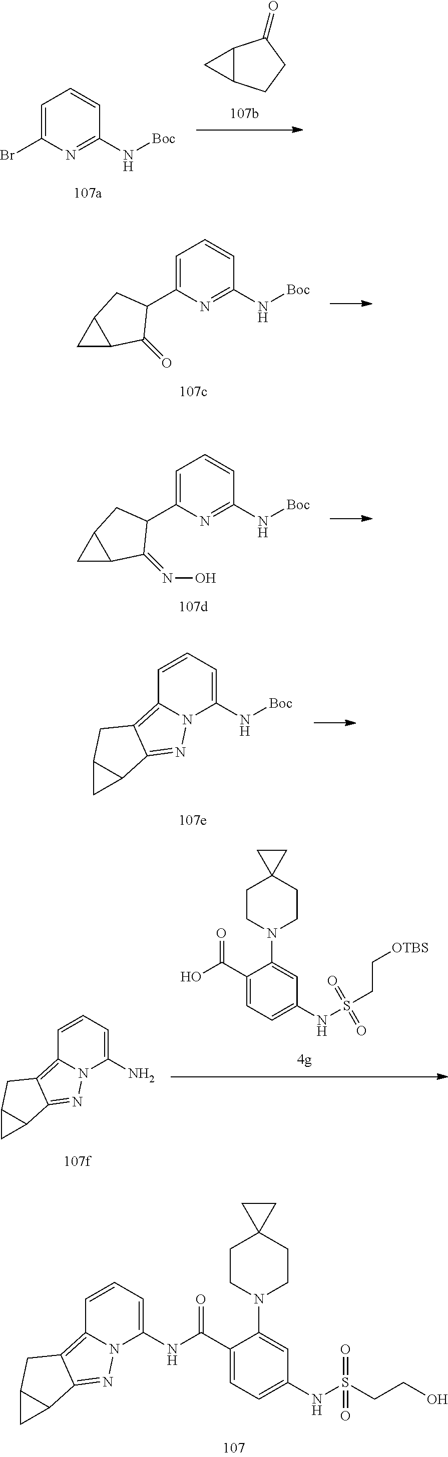

### Step 1: Synthesis of Compound 107c

Compound 107c (1.04 g) was obtained through synthesis and purification using a method identical to that described in step 1 of Example 64, with compound 107a (1.36 g, 5.0 mmol) and compound 107b (0.58 g, 6.0 mmol) as starting materials.

### Step 2: Synthesis of Compound 107d

Compound 107d (0.85 g) was obtained through synthesis and purification using a method identical to that described in step 2 of Example 64, with compound 107c (1.0 g, 3.5 mmol) as starting material.

### Step 3: Synthesis of Compound 107e

Compound 107e (0.48 g) was obtained through synthesis and purification using a method identical to that described in step 3 of Example 64, with compound 107d (0.84 g, 2.8 mmol) as starting material.

### Step 4: Synthesis of Compound 107f

Compound 107f (290 mg) was obtained through synthesis and purification using a method identical to that described in step 5 of Example 30, with compound 107e (480 mg, 1.7 mmol) as starting material.

MS m/z (ESI): 186.1 [M+H]⁺.

### Step 5: Synthesis of Compound 107

Compound 107 (0.8 mg) was obtained through synthesis and purification using a method identical to that described in step 5 of Example 23, with compound 107f (18.6 mg, 0.1 mmol) and compound 4g (48 mg, 0.1 mmol) as starting materials.

MS m/z (ESI): 522.2 [M+H]⁺.

### Example 109: Synthesis of Compound 109

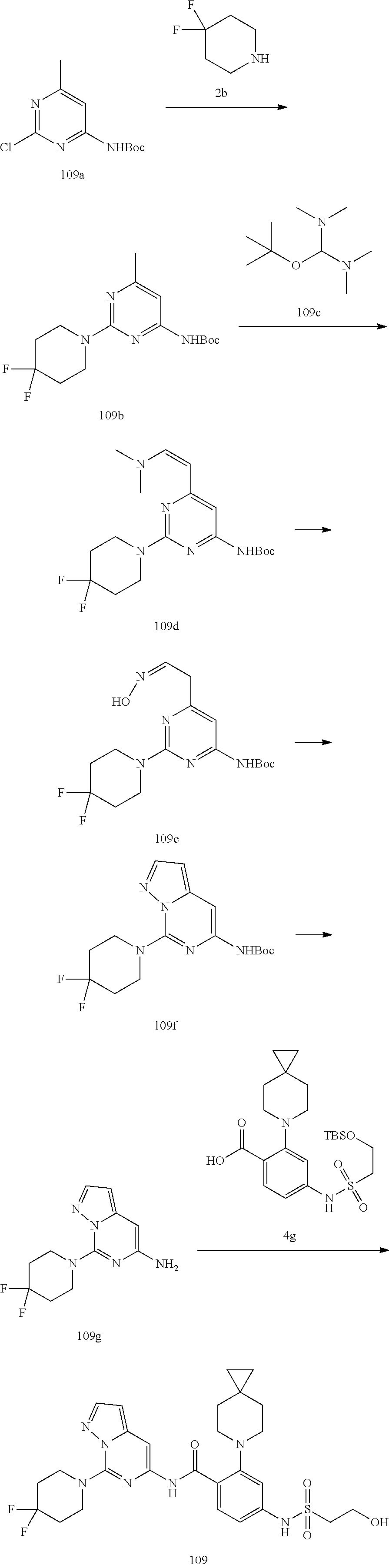

### Step 1: Synthesis of Compound 109b

Compound 109b (224 mg) was obtained through synthesis and purification using a method identical to that described in step 1 of Example 30, with compound 109a (0.20 g, 0.82 mmol) and compound 2a (hydrochloride, 0.19 g, 1.23 mmol) as starting materials.

### Step 2: Synthesis of Compound 109d

Compound 109b (150 mg, 0.46 mmol) was added to compound 109c (1.5 mL), and the mixture was reacted sufficiently at 120 °C. The volatiles were removed under vacuum, and the residue was purified by flash reverse-phase column chromatography (acetonitrile:water=30:70) to give compound 109d (40 mg).

### Step 3: Synthesis of Compound 109e

Compound 109e (41 mg) was obtained through synthesis and purification using a method identical to that described in step 2 of Example 64, with compound 109d (40 mg, 0.1 mmol) as starting material.

### Step 4: Synthesis of Compound 109f

Compound 109f (7.8 mg) was obtained through synthesis and purification using a method identical to that described in step 3 of Example 64, with compound 109e (41 g, 0.1 mmol) as starting material.

### Step 5: Synthesis of Compound 109g

Compound 109g (3 mg) was obtained through synthesis and purification using a method identical to that described in step 5 of Example 30, with compound 109f (7.8 mg, 0.02 mmol) as starting material.

MS m/z (ESI): 254.1 [M+H]⁺.

### Step 6: Synthesis of Compound 109

Compound 109 (1 mg) was obtained through synthesis and purification using a method identical to that described in step 5 of Example 23, with compound 109g (3 mg, 0.01 mmol) and compound 4g (9.6 mg, 0.2 mmol) as starting materials.

MS m/z (ESI): 590.4 [M+H]⁺.

### Example 110: Synthesis of Compound 110

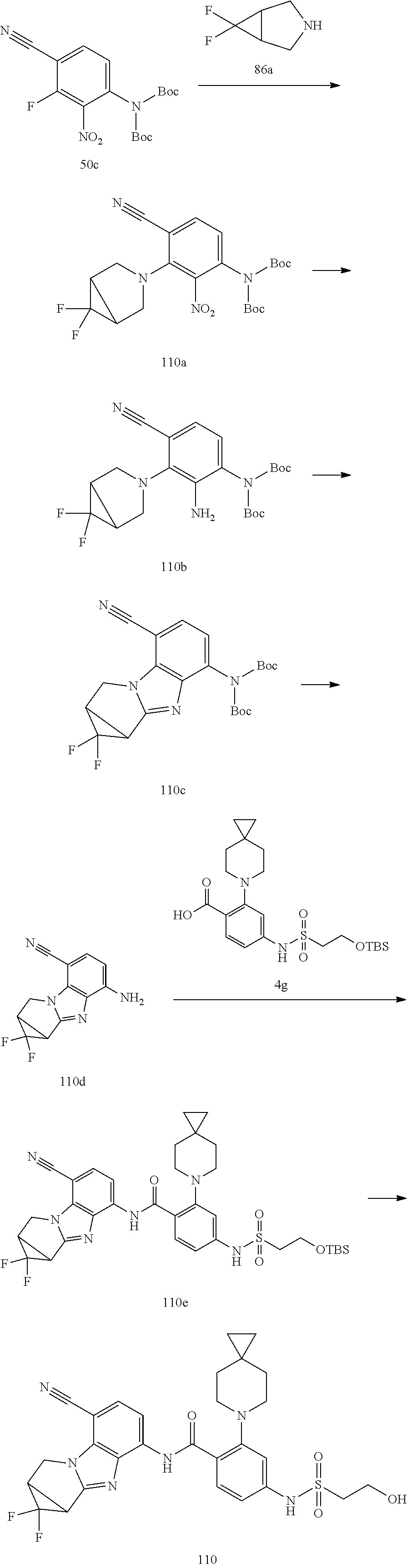

### Step 1: Synthesis of Compound 110a

Compound 110a (208 mg) was obtained through synthesis and purification using a method identical to that described in step 1 of Example 30, with compound 50c (245 mg, 0.64 mmol) and compound 86a (100 mg, 0.64 mmol) as starting materials.

### Step 2: Synthesis of Compound 110b

Compound 110b (155 mg) was obtained through synthesis and purification using a method identical to that described in step 3 of Example 4, with compound 110a (200 mg, 0.42 mmol) as starting material.

### Step 3: Synthesis of Compound 110c

Compound 110c (90 mg) was obtained through synthesis and purification using a method identical to that described in step 3 of Example 30, with compound 110b (155 mg, 0.44 mmol) as starting material.

### Step 4: Synthesis of Compound 110d

Compound 110d (43 mg) was obtained through synthesis and purification using a method identical to that described in step 5 of Example 50, with compound 110c (90 mg, 0.26 mmol) as starting material.

MS m/z (ESI): 247.2 [M+H]⁺.

### Step 5: Synthesis of Compound 110e

Compound 110e (12 mg) was obtained through synthesis and purification using a method identical to that described in step 3 of Example 5, with compound 4g (70 mg, 0.15 mmol) and 110d (25 mg, 0.1 mmol) as starting materials.

### Step 6: Synthesis of Compound 110

Compound 110 (7.8 mg) was obtained through synthesis and purification using a method identical to that described in step 8 of Example 4, with compound 110e (11.8 mg, 17 μmol) as starting material.

MS m/z (ESI): 583.4 [M+H]⁺.

¹H NMR (400 MHz, DMSO-d₆) δ 12.78 (s, 1H), 8.46 (d, J=8.6 Hz, 1H), 8.03 (d, J=8.6 Hz, 1H), 7.72 (d, J=8.5 Hz, 1H), 7.26 (dd, J=8.6, 2.1 Hz, 1H), 4.81 (dd, J=11.8, 6.6 Hz, 1H), 4.61 (d, J=11.8 Hz, 1H), 3.85 (t, J=9.1 Hz, 1H), 3.77 (t, J=6.5 Hz, 2H), 3.57-3.70 (m, 1H), 3.36 (t, J=6.5 Hz, 2H), 3.06-2.97 (m, 4H), 1.76 (br, 4H), 0.31 (s, 4H).

### Example 112: Synthesis of Compound 112

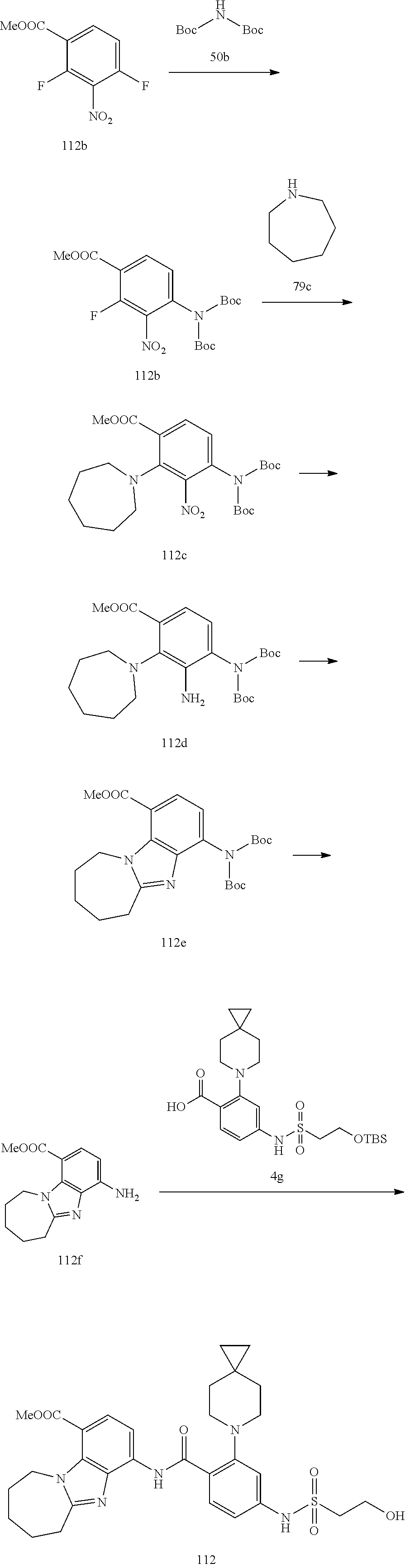

### Step 1: Synthesis of Compound 112b

Compound 112b (678 mg) was obtained through synthesis and purification using a method identical to that described in step 1 of Example 50, with compound 112a (600 mg, 2.76 mmol) and compound 50b (651 mg, 3.0 mmol) as starting materials.

### Step 2: Synthesis of Compound 112c

Compound 112c (623 mg) was obtained through synthesis and purification using a method identical to that described in step 1 of Example 30, with compound 112b (660 mg, 1.59 mmol) and compound 79c (198 mg, 2.0 mmol) as starting materials.

### Step 3: Synthesis of Compound 112d

Compound 112d (482 mg) was obtained through synthesis and purification using a method identical to that described in step 3 of Example 4, with compound 112c (600 mg, 1.22 mmol) as starting material.

### Step 4: Synthesis of Compound 112e

Compound 112e (212 mg) was obtained through synthesis and purification using a method identical to that described in step 3 of Example 30, with compound 112d (460 mg, 1.0 mmol) as starting material.

### Step 5: Synthesis of Compound 112f

Compound 112f (34 mg) was obtained through synthesis and purification using a method identical to that described in step 5 of Example 50, with compound 112e (100 mg, 0.22 mmol) as starting material.

MS m/z (ESI): 260.2 [M+H]⁺.

### Step 6: Synthesis of Compound 112

Compound 112 (5.4 mg) was obtained through synthesis and purification using a method identical to that described in step 5 of Example 23, with compound 4g (48 mg, 0.10 mmol) and 112f (26 mg, 0.1 mmol) as starting materials.

MS m/z (ESI): 596.3 [M+H]⁺.

¹H NMR (400 MHz, DMSO-d₆) δ 12.66 (s, 1H), 8.37 (d, J=8.6 Hz, 1H), 8.05 (d, J=8.6 Hz, 1H), 7.70 (d, J=8.6 Hz, 1H), 7.28 (d, J=2.1 Hz, 1H), 7.10 (dd, J=8.5, 2.1 Hz, 1H), 4.51 (d, J=6.9 Hz, 2H), 3.89 (s, 3H), 3.76 (t, J=6.5 Hz, 2H), 3.36 (t, J=6.6 Hz, 2H), 3.15 (d, J=8.0 Hz, 2H), 3.07-2.95 (m, 4H), 1.90-1.69 (m, 8H), 0.31 (s, 4H).

### Example 113: Synthesis of Compound 113

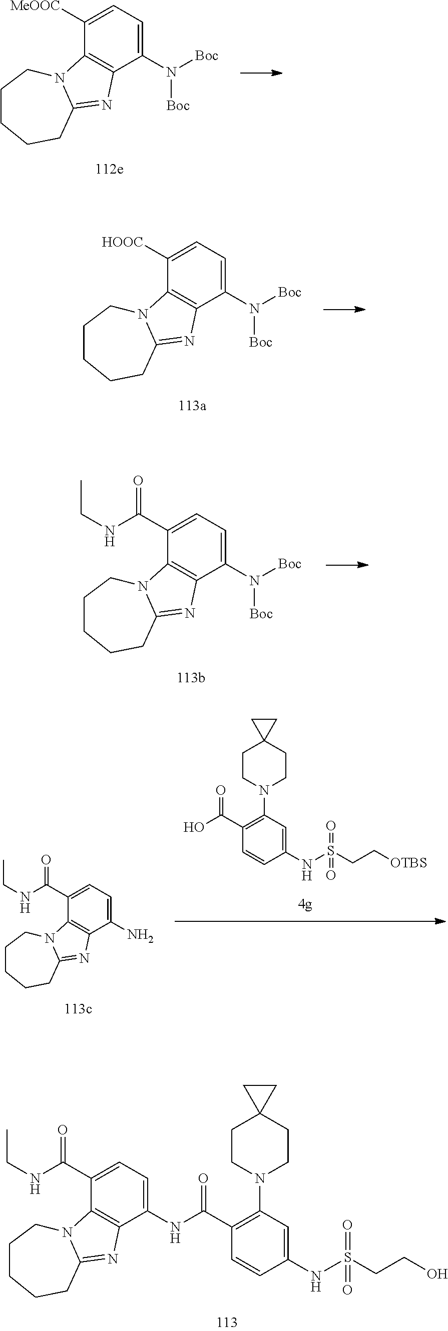 [SMILES: COC(=O)c1ccc(N(C(=O)OC(C)(C)C)C(=O)OC(C)(C)C)c2nc3n(c12)CCCCC3 ; CC(C)(C)OC(=O)N(C(=O)OC(C)(C)C)c1ccc(C(=O)O)c2c1nc1n2CCCCC1 ; CCNC(=O)c1ccc(N(C(=O)OC(C)(C)C)C(=O)OC(C)(C)C)c2nc3n(c12)CCCCC3 ; CCNC(=O)c1ccc(N)c2nc3n(c12)CCCCC3 ; CC(C)(C)[Si](C)(C)OCCS(=O)(=O)Nc1ccc(C(=O)O)c(N2CCC3(CC2)CC3)c1 ; CCNC(=O)c1ccc(NC(=O)c2ccc(NS(=O)(=O)CCO)cc2N2CCC3(CC2)CC3)c2nc3n(c12)CCCCC3]

### Step 1: Synthesis of Compound 113a

Compound 113a (90 mg) was obtained through synthesis and purification using a method identical to that described in step 2 of Example 45, with compound 112e (100 mg, 0.22 mmol) as starting material.

### Step 2: Synthesis of Compound 113b

Compound 113b (58 mg) was obtained through synthesis and purification using a method identical to that described in step 2 of Example 1, with compound 113a (90 mg, 0.2 mmol) and ethylamine hydrochloride (33 mg, 0.4 mmol) as starting materials.

### Step 3: Synthesis of Compound 113c

Compound 113c (14 mg) was obtained through synthesis and purification using a method identical to that described in step 5 of Example 50, with compound 113b (50 mg, 0.1 mmol) as starting material.

MS m/z (ESI): 273.2 [M+H]⁺.

### Step 4: Synthesis of Compound 113

Compound 113 (4.3 mg) was obtained through synthesis and purification using a method identical to that described in step 5 of Example 23, with compound 4g (48 mg, 0.10 mmol) and 113c (13 mg, 0.05 mmol) as starting materials.

MS m/z (ESI): 609.3 [M+H]⁺.

¹H NMR (400 MHz, DMSO-d₆) δ 12.50 (s, 1H), 10.14 (br, 1H), 8.54 (d, J=5.6 Hz, 1H), 8.29 (d, J=8.2 Hz, 1H), 8.04 (d, J=8.6 Hz, 1H), 7.27 (d, J=2.1 Hz, 1H), 7.19 (d, J=8.2 Hz, 1H), 7.09 (dd, J=8.6, 2.1 Hz, 1H), 3.92 (t, J=5.7 Hz, 2H), 4.34 (d, J=8.0 Hz, 2H), 3.76 (q, J=6.1 Hz, 2H), 3.36 (t, J=6.7 Hz, 2H), 3.12 (d, J=8.0 Hz, 2H), 2.99 (br, 4H), 1.95-1.68 (m, 8H), 1.15 (t, J=7.2 Hz, 3H), 0.30 (s, 4H).

### Example 114: Synthesis of Compound 114

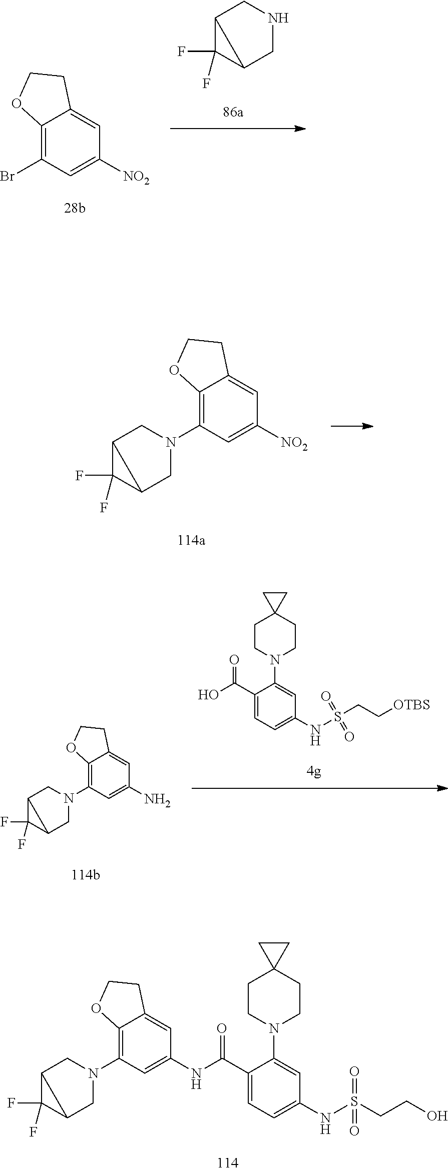 [SMILES: O=[N+]([O-])c1cc(Br)c2c(c1)CCO2 ; FC1(F)C2CNCC21 ; O=[N+]([O-])c1cc2c(c(N3CC4C(C3)C4(F)F)c1)OCC2 ; Nc1cc2c(c(N3CC4C(C3)C4(F)F)c1)OCC2 ; CC(C)(C)[Si](C)(C)OCCS(=O)(=O)Nc1ccc(C(=O)O)c(N2CCC3(CC2)CC3)c1 ; O=C(Nc1cc2c(c(N3CC4C(C3)C4(F)F)c1)OCC2)c1ccc(NS(=O)(=O)CCO)cc1N1CCC2(CC1)CC2]

### Step 1: Synthesis of Compound 114a

Compound 114a (71 mg) was obtained through synthesis and purification using a method identical to that described in step 4 of Example 22, with compound 28b (100 mg, 0.41 mmol) and 86a (hydrochloride, 61 mg, 0.41 mmol) as starting materials.

### Step 2: Synthesis of Compound 114b

Compound 114b (47 mg) was obtained through synthesis and purification using a method identical to that described in step 3 of Example 4, with compound 114a (70 mg, 0.25 mmol) as starting material.

MS m/z (ESI): 253.3 [M+H]⁺.

### Step 3: Synthesis of Compound 114

Compound 114 (18.0 mg) was obtained through synthesis and purification using a method identical to that described in step 5 of Example 23, with compound 114b (25 mg, 0.1 mmol) and compound 4g (48 mg, 0.1 mmol) as starting materials.

MS m/z (ESI): 589.5 [M+H]⁺.

### Example 115: Synthesis of Compound 115

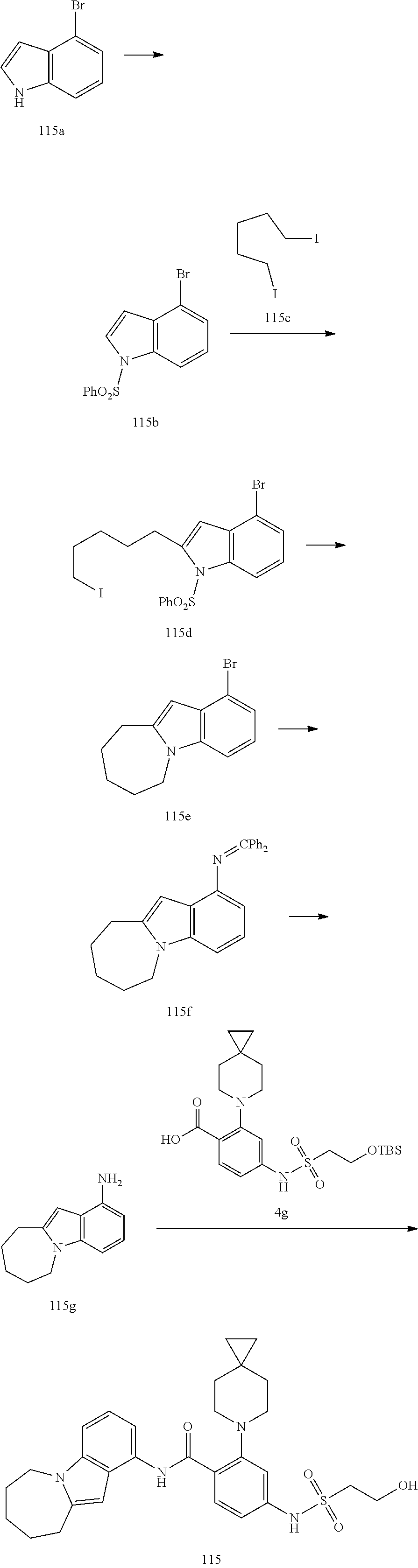

### Step 1: Synthesis of Compound 115b

Compound 115b (17.0 g) was obtained through synthesis and purification using a method identical to that described in step 1 of Example 26, with compound 115a (15 g, 76.5 mmol) as starting material.

### Step 2: Synthesis of Compound 115d

Compound 115d (5.5 g) was obtained through synthesis and purification using a method identical to that described in step 2 of Example 26, with compound 115b (5.1 g, 15.2 mmol) and 115c (5.4 g, 16.7 mmol) as starting materials.

### Step 3: Synthesis of Compound 115e

Compound 115e (2.0 g) was obtained through synthesis and purification using a method identical to that described in step 3 of Example 26, with compound 115d (5.3 mg, 10.0 mmol) as starting material.

### Step 4: Synthesis of Compound 115f

Compound 115f (1.05 g) was obtained through synthesis and purification using a method identical to that described in step 4 of Example 30, with compound 115e (1.0 g, 3.8 mmol) and benzophenone imine (820 mg, 4.5 mmol) as starting materials.

### Step 5: Synthesis of Compound 115g

Compound 115g (168 mg) was obtained through synthesis and purification using a method identical to that described in step 5 of Example 30, with compound 115f (1.0 g, 2.7 mmol) as starting material.

MS m/z (ESI): 201.1 [M+H]⁺.

### Step 6: Synthesis of Compound 115

Compound 115 (12.1 mg) was obtained through synthesis and purification using a method identical to that described in step 5 of Example 23, with compound 115g (96 mg, 0.2 mmol) and compound 4g (40 mg, 0.2 mmol) as starting materials.

MS m/z (ESI): 537.2 [M+H]⁺.

¹H NMR (400 MHz, DMSO-d₆) δ 11.16 (s, 1H), 10.06 (s, 1H), 7.90 (d, J=8.5 Hz, 1H), 7.84 (d, J=7.7 Hz, 1H), 7.25-7.17 (m, 2H), 7.10-7.01 (m, 2H), 6.45 (s, 1H), 4.96 (s, 1H), 4.36-4.17 (m, 2H), 3.78 (t, J=6.6 Hz, 2H), 3.39-3.37 (m, 2H), 3.09-2.98 (m, 4H), 2.95-2.86 (m, 2H), 1.87-1.76 (m, 2H), 1.74-1.59 (m, 4H), 1.56-1.42 (m, 4H), 0.29 (s, 4H).

### Evaluation of Biological Activity

### Test Example 1: KIF18A Enzyme Activity Assay

Based on the ADP-Glo™ Kinase Assay, the ADP amount generated in the reaction was detected to reflect the enzyme activity of KIF18A.

### 1. Experimental Instruments and Materials

| Instrument | Manufacturer | Model |
|---|---|---|
| Oscillator | Boxun | BSD-YX3400 |
| Plate reader | PerkinElmer | Envision |
| Centrifuge | Eppendorf | Eppendorf Mixmate |
| Compound dilution and | PerkinElmer | Echo |
| sample loading instrument | | |

Human KIF18A (1-376aa) protease used in the experiments, expressed by Shanghai Panchao Biotechnology Co., Ltd., was stored at −80 °C (no more than 5 freeze-thaw cycles).

The assay kit (ADP-Glo™ Kinase Assay) was purchased from Promega (Cat. No. V9101) and stored at −30 °C. The kit detects the enzyme activity by measuring ADP generated in enzyme reaction. The KIF18A enzyme and ATP generate ADP. The remaining ATP in this reaction is consumed by the ADP-Glo reagent, while the ADP generated by the KIF18A enzyme reaction is converted back to ATP by the assay reagent. The ATP then reacts with luciferin in the presence of Ultra-Glo™ luciferase, producing luminescence. The luminescent signal is positively correlated with the activity of the KIF18A enzyme.

The other reagents and consumables required for the experiment are as follows

| Reagent | Brand | Cat. No. |
|---|---|---|
| ATP | Promega | V915A |
| Tris | Teknova Inc | T0664 |
| Pluronic F-68 | Thermo Fisher | 24040032 |
| Microtubule | Cytoskeleton | MT002 |
| Taxol | Cytoskeleton | TXD01 |
| Reagent reservoir | Corning | 4877 |
| White 384-well plate | Corning | 3765 |

### 2. Experimental Procedures

Compounds were each subjected to 3-fold serial dilution starting from 10 μM to obtain 10 concentration points. The compounds and pure DMSO (control) were pipetted into wells of a 384-well plate using an Echo instrument, and the total volume of compound and DMSO was 100 nL; sample concentrations of gradient dilution were obtained by the instrument based on different ratios. Preparation of a KIF18A enzyme reaction buffer: 15 nM Tris, 10 mM MgCl₂, 0.01% Pluronic F-68, 1 μM Taxol, and 50 μg/mL Microtubule. KIF18A and the enzyme reaction buffer were mixed, and then the mixed solution was added to the 384-well plate and incubated at room temperature for 15 min. ATP (Km 70 μM) was then added to the mixed solution, and the mixed solution was incubated at room temperature for 15 min. 10 μL of ADP-Glo™ Reagent was well mixed with 10 μL of the mixed solution, and the mixture was incubated at room temperature for 40 min. Finally, 20 μL of ADP-Glo™ Detection Reagent was added, and the mixture was incubated at room temperature for 30 min. The chemiluminescent signal in each well was measured using an Envision plate reader (PerkinElmer, emission wavelength: 400-700 nm). In the experiment, 1 column of wells with no compound and no enzyme (chemiluminescence value was taken as [RLU]background) and 1 column of wells with enzyme but no compound (chemiluminescence value was taken as [RLU]enzyme) were provided. The chemiluminescence value of the compound adding group was taken as [RLU]cpd. The inhibition rate of the compound on proliferation was calculated according to the following equation: inhibition rate (%)=([RLU]enzyme−[RLU]cpd)/([RLU]enzyme−[RLU]background)×100%, and the inhibitory activity IC₅₀ value of the compound on the enzyme activity was calculated using a four-parameter Logistic Model method. In the following equation, x represents the logarithmic form of the concentration of compound, and f (x) represents an effect value (inhibition rate of enzyme activity at the concentration): F(x)=(A+((B−A)/(1+((C/x)\hat{ }D)))). A. B, C, and D are four parameters. IC₅₀ values were further calculated as the concentration of compound required for 50% inhibition of enzyme activity in the best-fit curve using Xlfit, and the KIF18A inhibitory activity of the compounds of the present disclosure is shown in Table 1.

TABLE 1
KIF18A inhibitory activity of compounds of the present disclosure

| | KIF18A inhibitory activity |
|---|---|
| Compound No. | IC₅₀ (nM) |
| 2 | ++++ |
| 3 | ++++ |
| 4 | ++++ |
| 5 | ++++ |
| 6 | ++++ |
| 7 | ++++ |
| 8 | ++++ |
| 9 | ++++ |
| 10 | ++++ |
| 12 | ++++ |
| 13 | ++++ |
| 14 | ++++ |
| 15 | ++++ |
| 16 | ++++ |
| 18 | ++++ |
| 19 | ++++ |
| 20 | ++++ |
| 22 | ++++ |
| 23 | ++++ |
| 24 | ++++ |
| 25 | ++++ |
| 26 | ++++ |
| 27 | ++++ |
| 28 | ++++ |
| 29 | ++++ |
| 30 | ++++ |
| 31 | ++++ |
| 32 | ++++ |
| 33 | ++++ |
| 34 | ++++ |
| 35 | ++++ |
| 36 | ++++ |
| 39 | ++++ |
| 40 | ++++ |
| 41 | ++++ |
| 42 | ++++ |
| 43 | +++ |
| 44 | ++++ |
| 45 | ++++ |
| 46 | ++++ |
| 47 | ++++ |
| 48 | ++++ |
| 49 | ++++ |
| 50 | ++++ |
| 51 | ++++ |
| 52 | ++++ |
| 53 | ++++ |
| 54 | ++++ |
| 55 | ++++ |
| 56 | ++++ |
| 57 | ++++ |
| 58 | ++++ |
| 59 | ++++ |
| 60 | ++++ |
| 61 | ++++ |
| 62 | ++++ |
| 63 | ++++ |
| 64 | ++++ |
| 65 | +++ |
| 66 | +++ |
| 67 | ++++ |
| 68 | ++++ |
| 69 | ++++ |
| 70 | ++++ |
| 71 | ++++ |
| 72 | ++++ |
| 73 | ++++ |
| 74 | ++++ |
| 75 | ++++ |
| 76 | ++++ |
| 77 | ++++ |
| 78 | ++++ |
| 79 | ++++ |
| 80 | ++++ |
| 81 | ++++ |
| 82 | ++++ |
| 83 | +++ |
| 84 | +++ |
| 85 | +++ |
| 86 | ++++ |
| 87 | ++++ |
| 88 | ++++ |
| 89 | +++ |
| 90 | ++++ |
| 91 | ++++ |
| 92 | ++++ |
| 93 | ++++ |
| 95 | ++++ |
| 96 | ++++ |
| 101 | ++++ |
| 103 | ++++ |
| 105 | ++++ |
| 106 | ++++ |
| 107 | ++++ |
| 109 | ++++ |
| 110 | ++++ |
| 112 | ++++ |
| 113 | ++++ |
| 114 | ++++ |
| 115 | ++++ |

In the above table, “++++” indicates that the test compound has an IC₅₀ range of <200 nM for enzyme inhibitory activity, and “+++” indicates that the test compound has an IC₅₀ range of <500 nM for enzyme inhibitory activity.

### Test Example 2. Test on Inhibition of Proliferation of OVCAR-3 Cells by Compounds of Present Disclosure

Cell and material: The human ovarian cancer cell line OVCAR3 was purchased from ATCC (Cat. No. HTB-161™), RPMI 1640 medium (Gibco #A1049101), penicillin-streptomycin (Gibco #15140122), and 0.25% Trypsin-EDTA (Gibco #25200056) were purchased from Gibco (U.S.), bovine insulin (Yeasen #40107ES60) was purchased from Yeasen, 384-well plates (Corning #CLS3765) was purchased from Corning (U.S.), and Cell-Titer Glo reagent (Promega #G7568) was purchased from Promega (U.S.).

Cell culture: OVCAR3 cells were cultured in RPMI 1640 complete medium (RPMI 1640 medium containing 20% fetal bovine serum, 10 g/mL bovine insulin, and 1% penicillin-streptomycin) in an incubator (37 °C, 5% CO₂), and the cells could be used in experiments when in the logarithmic growth phase.

Cell proliferation activity test: The Cell-Titer Glo reagent was used to test the compound for inhibition of proliferation of the OVCAR3 cell strain. The compound formulated with DMSO and pure DMSO (control) were each added to a 384-well plate using an Echo instrument; the compound was subjected to 3-fold serial dilution starting from 30 μM to obtain 11 concentration points, and the volume of the compound or DMSO added was 100 nL.

OVCAR3 cells were digested and resuspended in RPMI 1640 complete medium, and the suspension was added to a 384-well plate (1000 cells/50 μL/well) to be well mixed with the compound. The mixture was incubated in an incubator (37 °C, 5% CO₂) for 3 days. The Cell-Titer Glo reagent was added at 25 L/well, and the resulting mixture was well mixed by shaking and then incubated for 10 min. The Cell-Titer Glo readings were measured with a Multimode Plate Reader (brand: PerkinElmer; model: SIC-PV-018).

A negative control group (Bottom) was set, and the negative control group was medium wells with 0.2% DMSO added and was defined as 100% proliferation inhibition; the positive control group (Top) was OVCAR3 cell wells with 0.2% DMSO added.

### Data Analysis:

The percent proliferation inhibition (% Inhibition) was calculated, and the curves were fit using the four-parameter equation, Y=Bottom+(Top−Bottom)/(1+(IC₅₀/X)\hat{ }HillSlope), to obtain the proliferation inhibition IC₅₀ of the compound.
Percent inhibition: % Inhibition=(1−(Signal−Bottom)/(Top−Bottom))×100%.
Signal: Cell-Titer Glo reading of wells with compound added;
Bottom: Cell-Titer Glo reading of negative control wells;
Top: Cell-Titer Glo reading of positive control wells.

### Experimental Results:

The inhibitory activity of the compounds of present disclosure on OVCAR3 proliferation is shown in Table 2.

TABLE 2
Inhibitory activity of compounds of the present
disclosure on OVCAR3 proliferation

| | OVCAR3 proliferation |
|---|---|
| Compound No. | inhibition IC₅₀ (nM) |
| 12 | 95.9 |
| 14 | 77.6 |
| 24 | 52 |
| 28 | 3.67 |
| 29 | 69.8 |
| 34 | 22.8 |
| 35 | 88 |
| 39 | 75.5 |
| 40 | 36.6 |
| 41 | 16.8 |
| 42 | 26.5 |
| 44 | 9.13 |
| 45 | 6.65 |
| 47 | 56.2 |
| 48 | 20.4 |
| 49 | 49.6 |
| 50 | 48.9 |
| 51 | 68.1 |
| 53 | 21 |
| 54 | 21 |
| 55 | 17.5 |
| 56 | 31.2 |
| 58 | 8 |
| 59 | 23.3 |
| 60 | 11 |
| 61 | 77 |
| 64 | 65.7 |
| 65 | 16.7 |
| 67 | 70 |
| 68 | 41.6 |
| 70 | 93.3 |
| 71 | 94.2 |
| 72 | 57.3 |
| 73 | 38 |
| 74 | 24 |
| 76 | 43.1 |
| 77 | 25.4 |
| 78 | 45.3 |
| 80 | 24 |
| 81 | 18.2 |
| 82 | 99.3 |
| 83 | 86 |
| 86 | 6.5 |
| 87 | 31 |
| 88 | 98 |
| 92 | 48.4 |
| 96 | 25 |
| 101 | 9.1 |
| 103 | 14.7 |
| 105 | 14.9 |
| 106 | 15.7 |
| 107 | 99.3 |
| 109 | 37.4 |
| 110 | 11.2 |
| 112 | 15.3 |
| 113 | 5.32 |
| 114 | 10.7 |
| 115 | 23 |

### Test Example 3: Test on Inhibition of CYP Enzyme by Compounds of Present Disclosure

The metabolic reactions of representative substrates of 5 major human CYP subtypes (CYP1A2, CYP2C9, CYP2C19, CYP2D6, and CYP3A4/5) were evaluated using 150-donor pooled human liver microsomes (purchased from Corning, Cat. No. 452117). The effects of different concentrations of the test compounds on the metabolic reactions of phenacetin (CYP1A2), diclofenac sodium (CYP2C9), S-mephenytoin (CYP2C19), bufuralol hydrochloride (CYP2D6), and midazolam (CYP3A4/5) were assessed by liquid chromatography-tandem mass spectrometry (LC/MS/MS).

30 μM phenacetin, 10 μM diclofenac sodium, 35 μM S-mephenytoin, 5 μM bufuralol hydrochloride, 3 μM midazolam, 1 mM reduced nicotinamide adenine dinucicotide phosphate (NADPH), and test compounds (at concentrations of 0.1 μmol/L, 0.3 μmol/L, 1 μmol/L, 3 μmol/L, 10 μmol/L, and 30 μmol/L, respectively) or positive compound or blank control were incubated with 200 μL of a reaction system of pooled human liver microsomes (0.2 mg/mL) (100 mmol/L phosphate-buffered saline, pH 7.4, containing 0.3 vol % of DMSO, 0.6 vol % of acetonitrile, and 0.1 vol % of methanol) at 37 °C for 5 min. Then, 200 μL of an acetonitrile solution containing 3% formic acid and 40 nM internal standard verapamil was added, and the mixture was centrifuged at 4000 rpm for 50 min. The mixture was cooled on ice for 20 min and centrifuged at 4000 rpm for 20 min to precipitate protein. 200 μL of the supernatant was taken for LC/MS/MS analysis.

The peak area was calculated based on the chromatogram.

The residual activity rate (%) was calculated using the following equation:

peak area rate=peak area of metabolite/peak area of internal standard

residual activity rate (%)=peak area rate of the test compound group/peak area rate of the blank group

The half maximal inhibitory concentrations (IC₅₀) against CYP were calculated using Excel XLfit 5.3.1.3.

### Test Example 4. Caco-2 Permeability Assay

The apparent permeability coefficient (Pₐₚₚ) of the analytical drug was determined by liquid chromatography-tandem mass spectrometry (LC/MS/MS) using a Caco-2 cell model.

In this test example: Caco-2 cells were purchased from American Type Culture Collection (ATCC); 4-hydroxyethyl piperazine ethane sulfonic acid (HEPES) was purchased from Beijing Solarbio Science & Technology Co., Ltd.; Hanks' balanced salt solution (HBSS) and non-essential amino acids (NEAA) were purchased from ThermoFisher Scientific; penicillin, streptomycin, and trypsin/EDTA were purchased from Solarbio; fetal bovine serum (FBS) and DMEM medium were purchased from Corning; HTS-96-well Transwell plates and other sterile consumables were purchased from Corning; Millicell resistance measuring system was purchased from Millipore; Cellometer®K2 was purchased from Nexcelom Bioscience; Infinite 200 PRO microplate reader was purchased from Tecan; MTS2/4 orbital shaker was purchased from IKA Labortechnik.

### Step 1. Cell Culture and Plating

Caco-2 was cultured in a cell culture flask. The incubator was set to be at 37 °C and 5% CO₂, with the relative humidity guaranteed to be 95%. Cells could be used to be inoculated in the Transwell when at a confluency of 70-90%. Prior to cell inoculation, 50 μL of cell medium was added to each well of the upper Transwell chamber and 25 mL of cell medium was added to the lower culture plate. The culture plate was incubated in an incubator (37 °C, 5% CO₂) for 1 h and then inoculated with cells. After cell digestion, the cell suspension was pipetted and transferred to a round-bottomed centrifuge tube for centrifugation at 120 g for 5 min. The cells were resuspended in the medium to a final concentration of 6.86×10⁵ cells/mL. The cell suspension was then added to the upper chamber of the 96-well Transwell culture plate at 50 μL/well, with a final inoculation density of 2.4×10⁵ cells/cm². The medium was first changed 24 h after inoculation; the cells were cultured for 14-18 days, and the medium was changed every other day. The medium was changed as follows: Transwell chambers were separated from the receiver plate, medium in the receiver plate was discarded, then medium in the Transwell chambers was discarded, and finally, 75 μL of fresh medium was added to each chamber, and 25 mL of fresh medium was added to the receiver plate.

### Step 2. Evaluation of Integrity of Monolayer Cell Membrane

After approximately 14 days of culture, the Caco-2 cells reached the confluency and completed differentiation. In this case, the cells could be used for permeability assay. The resistance of the monolayer membrane was measured using a resistance meter (Millipore, USA), and the resistance of each well was recorded. After the measurement, the Transwell culture plate was put back into the incubator. Calculation of resistance: measured resistance value (ohms)×membrane area (cm²)=TEER value (ohm·cm²); if TEER value<230 ohms·cm², the well could not be used for permeability assay.

### Step 3. Preparation of Solution

2.38 g of HEPES and 0.35 g of sodium bicarbonate were dissolved in 900 mL of purified water, and then 100 mL of 10×HBSS was added. The mixture was stirred well, and the pH was adjusted to 7.4. Finally, the mixture was filtered to obtain 1 L of transport buffer (HBSS, 10 mM HEPES, pH 7.4).

A 1 mM DMSO stock solution of test compound was diluted with the transport buffer to obtain 5 μM test solution. The control compound digoxin or minoxidil was diluted with DMSO to 2 mM and then diluted with the transport buffer described above to 10 μM to obtain a control compound test solution. In addition, DMSO was also diluted with the transport buffer described above to obtain a receiving end solution containing 0.5% DMSO.

### Step 4. Drug Permeability Assay

The Transwell culture plate was taken out from the incubator. The monolayer cell membrane was rinsed twice with the transport buffer (10 mM HEPES, pH 7.4) and incubated at 37 °C for 30 min.

The transport rate of the compound from the apical side to the basolateral side was determined. 125 μL of test solution was added to each well of the upper chamber (apical side), and then 50 μL of solution was immediately transferred from the apical side to 200 μL of acetonitrile containing an internal standard (0.1 μM tolbutamide) to serve as an initial sample from the apical side to the basolateral side. 235 μL of receiving end solution was added to each well of the lower chamber (basolateral side).

The transport rate of the compound from the basolateral side to the apical side was determined. 285 μL of receiving end solution was added to each well of the upper chamber (apical side), and then 50 μL of solution was immediately transferred from the apical side to 200 μL of acetonitrile containing an internal standard (0.1 μM tolbutamide) to serve as an initial sample from the basolateral side to the apical side. 75 μL of test solution was added to each well of the lower chamber (basolateral side).

After the combination of the upper and lower transport devices, incubation was performed at 37 °C for 2 h.

After incubation, 50 μL of sample were taken from each well of the upper and lower chambers of the Transwell culture plate to new sample tubes. 200 μL of acetonitrile containing an internal standard (0.1 μM tolbutamide) was added to each sample tube, and then the sample tube was vortexed for 10 min and centrifuged at 3220 g for 40 min. Then, 150 μL of supernatant was pipetted and diluted with 150 μL of water for LC-MS/MS analysis. All samples were prepared in triplicate.

The integrity of monolayer cell membranes after 2 h of incubation was evaluated by leakage of fluorescein, and the fluorescein stock solution was diluted with the transport buffer (10 mM HEPES, pH 7.4) to a final concentration of 100 μM. 100 μL of fluorescein solution was added to each well of the upper Transwell plate, and 300 μL of transport buffer (10 mM HEPES, pH 7.4) was added to each well of the lower receiver plate. After incubation at 37 °C for 30 min, 80 μL of solution was pipetted from the upper and lower layers of each well into a new 96-well plate. Fluoremetry was performed using a microplate reader at an excitation wavelength of 485 nm and an emission wavelength of 530 nm.

### Step 5. Data Analysis

All calculations were performed using Microsoft Excel. Peak areas were determined by using extracted ion chromatograms.

The apparent permeability coefficient (Pₐₚₚ, cm/s×10⁻⁶) was calculated according to the following equation:

$${P}_{app} = \frac{{V}_{A}}{Area \times time} \times \frac{{[ drug ]}_{r e c e i v e r}}{{[ drug ]}_{initial , donor}}$$

In the equation: V_{A} was the volume of the receiving end solution (Ap→Bl was 0.3 mL, and Bl→Ap was 0.1 mL); Area was the area of the membrane of the Transwell 96-well plate (0.143 cm²); time was the time for incubation (s); [drug]_{receiver} was the concentration of the drug at the receiving end; [drug]_{initial, donor} was the initial concentration of the drug at the administration end.

The efflux ratio was calculated according to the following equation:

$$Efflux ratio = \frac{{P}_{app ( B \to A )}}{{P}_{app ( A \to B )}}$$

In the equation, P_{app (B-A)} was the apparent permeability coefficient from the basolateral side to the apical side, and

P_{app (A-B)} was the apparent permeability coefficient from the apical side to the basolateral side.

The recovery rate (“Percentage recovery” (%)) was calculated according to the following equation:

$$Percentage recovery ( \% ) = \frac{{[ drug ]}_{r eceive {r}^{\times }} {V}_{A} + {[ drug ]}_{d o n o r} \times {V}_{D}}{{[ drug ]}_{initial , donor} \times {V}_{D}} \times 100 \%$$

In the equation, V_{A} was the solution volume at the receiving end (mL), and V_{D} was the solution volume at the administration end (mL).

The leakage rate (Percentage leakage (%) or LY (%)) was calculated according to the following equation:

$$Percentage LY leakage ( \% ) = ( \frac{{I}_{receiver} \times 0.3}{{I}_{receiver} \times 0.3 + {I}_{d o n o r} \times 0.1} ) \times 100 \%$$

In the equation, I_{receiver} represented the fluorescence density of the receiving wells (0.3 mL), and I_{donor} represented the fluorescence density of the dosing wells (0.1 mL); the leakage rate was expressed as LY (%). LY<1.5% indicated that the monolayer cell membrane was intact. For individual LY>1.5% cases, the final data could be accepted based on scientific judgment if the Pₐₚₚ value was close to that in other replicates.

### Test Example 5: Assay for In Vitro Metabolic Stability in Rat Hepatocytes

The concentrations of the compounds in the reaction system were determined by LC/MS/MS, such that the intrinsic clearance of the test compounds was calculated, and the in vitro metabolic stability in rat hepatocytes was evaluated.

198 μL of 0.5×10⁶ cells/mL rat hepatocyte mixture and 2.0 μL of 100 μM test compound or positive control were added to the incubation plate to initiate reaction. Incubation was performed at 37 °C and 900 rpm. 25 μL of incubation system was transferred to a stop plate (150 μL of acetonitrile containing 100 nM alprazolam, 200 nM caffeine, and 100 nM toluenesulfonamide per well) at 0 min, 15 min, 30 min, 60 min, 90 min, and 120 min. Then, the mixture was vortexed for 5 min to be mixed well. The stop plate was centrifuged at 3220 g for 45 min. 100 μL of supernatant of each compound was transferred to a 96-well feeding plate, after which 100 μL of purified water was added to dilute the samples.

The resulting samples were quantified from ion chromatogram. The residual rate was calculated from the peak area of the test compound or the positive control. Slope k was determined by linear regression of the natural logarithm of the residual rate values versus incubation time using Microsoft Excel.

The intrinsic clearance (in vitro CLᵢₙₜ, μL/min/10⁶ cells) was calculated from the slope value k according to the following equation:

in vitro CLᵢₙₜ=−kV/N
V=incubation volume (0.25 mL);
N=number of cells per well (0.125×10⁶ cells)

### Test Example 6. Assay on In Vitro Metabolic Stability in Human Hepatocytes

The concentrations of the compounds in the reaction system were determined by LC/MS/MS, such that the intrinsic clearance of the test compounds was calculated, and the in vitro metabolic stability in human hepatocytes was evaluated.

198 μL of 0.5×10⁶ cells/mL human hepatocyte mixture and 2.0 μL of 100 μM test compound or positive control were added to the incubation plate to initiate reaction. Incubation was performed at 37 °C and 900 rpm. 25 μL of incubation system was transferred to a stop plate (150 μL of acetonitrile containing 100 nM alprazolam, 200 nM caffeine, and 100 nM toluenesulfonamide per well) at 0 min, 15 min, 30 min, 60 min, 90 min, and 120 min. Then, the mixture was vortexed for 5 min to be mixed well. The stop plate was centrifuged at 3220 g for 45 min. 100 μL of supernatant of each compound was transferred to a 96-well feeding plate, after which 100 μL of purified water was added to dilute the samples.

The resulting samples were quantified from ion chromatogram. The residual rate was calculated from the peak area of the test compound or the positive control. Slope k was determined by linear regression of the natural logarithm of the residual rate values versus incubation time using Microsoft Excel.

The intrinsic clearance (in vitro CLᵢₙₜ, μL/min/10⁶ cells) was calculated from the slope value according to the following equation:

in vitro CLᵢₙₜ=−kV/N
V=incubation volume (0.25 mL);
N=number of cells per well (0.125×10⁶ cells)

### Test Example 7: Assay on Solubility (PBS pH 7.4) of Compound Solids

The solubility of test compound solids in PBS (pH 7.4) was determined by LC/MS/MS.

Approximately 1 mg of powder of each compound was accurately weighed out and placed in a glass vial, and DMSO was added in a volume of 1 mL per milligram of compound. A stir bar was added to each vial, and the solubility sample vial was shaken at 25 °C and 1100 rpm for 2 h to completely dissolve the powder and thereby to prepare 1 mg/mL test sample solution. 5 μL of 1 mg/mL solution and 5 μL of PBS pH 7.4 solution were mixed in 490 μL of water and acetonitrile (1:1) containing an internal standard to prepare 10 μg/mL standard concentration solution of test sample. Then, 10 μL of 10 μg/mL solution was diluted in 90 μL of water and acetonitrile (1:1) containing an internal standard to prepare 1 g/mL standard concentration solution of test sample. The dilution factor of the standard solution could be adjusted according to the strength of response signals in LC/MS.

Approximately 1 mg of powder of each compound was accurately weighed out and placed in a glass vial, and the PBS pH 7.4 solution was added in a volume of 1 mL per milligram of compound. A stir bar was added to each vial, and the solubility sample vial was shaken at 25 °C and 1100 rpm for 24 h. After shaking, the stir bar was taken out, and the sample was transferred to a filter plate and filtered using a vacuum manifold. The filtrate resulting from filtration was diluted with a mixture of water and acetonitrile (1:1) containing an internal standard. The dilution factor could be adjusted according to the solubility value and the strength of response signals in LC/MS.

The resulting samples were determined by LC/MS/MS. The sample solubility was calculated from peak areas of the test compound solution and the standard concentration solution. The calculation equation is as follows:

$$[ Sample ] = \frac{Area {ratio}_{sample} \times INJ VOL STD \times {DF}_{sample} \times [ STD ]}{Area ratio STD \times INJ {VOL}_{Sample}}$$

[Sample] was the solubility of the test sample;
Area ratioₛₐₘₚₗₑ was the ratio of the peak area of sample to the peak area of the internal standard in the test sample;
INJ VOL STD was the sample injection volume of the standard concentration solution;
DFₛₐₘₚₗₑ was the dilution factor of the test sample solution;
[STD] was the concentration of the standard concentration solution;
INJ VOLₛₐₘₚₗₑ was the sample injection volume of the test sample solution;
Area ratio STD was the ratio of the peak area of the sample to the peak area of the internal standard in the standard concentration solution.

### Test Example 8: Assay on Solubility (PBS pH 7.4) of Compounds

The solubility of test compounds in PBS (pH 7.4) was determined by LC/MS/MS.

6 μL of 10 mM DMSO solution of the test compound was mixed with 194 μL of DMSO to prepare 300 μM compound solution. 5 μL of the solution and 5 μL of PBS pH 7.4 solution were mixed in 490 μL of water and acetonitrile (1:1) containing an internal standard to prepare 3 μM standard concentration solution of test sample. The dilution factor of the standard solution could be adjusted according to the strength of response signals in LC/MS.

15 μL of 10 mM DMSO solution of the test compound was added to 485 μL of PBS pH 7.4 solution. A stir bar was added, and the solubility sample vial was shaken at 25 °C and 1100 rpm for 2 h. After shaking, the stir bar was taken out, and the sample was transferred to a filter plate and filtered using a vacuum manifold. 5 μL of the filtrate and 5 μL of PBS pH 7.4 solution were mixed in 490 μL of water and acetonitrile (1:1) containing an internal standard to prepare a test solution. The dilution factor could be adjusted according to the solubility value and the strength of response signals in LC/MS.

The resulting samples were determined by LC/MS/MS. The sample solubility was calculated from peak areas of the test compound solution and the standard concentration solution. The calculation equation is as follows:

$$[ Sample ] = \frac{Area {ratio}_{sample} \times INJ VOL STD \times {DF}_{sample} \times [ STD ]}{Area ratio STD \times INJ {VOL}_{Sample}}$$

[Sample] was the solubility of the test sample;
Area ratioₛₐₘₚₗₑ was the ratio of the peak area of sample to the peak area of the internal standard in the test sample;
INJ VOL STD was the sample injection volume of the standard concentration solution;
DFₛₐₘₚₗₑ was the dilution factor of the test sample solution;
[STD] was the concentration of the standard concentration solution;
INJ VOLₛₐₘₚₗₑ was the sample injection volume of the test sample solution;
Area ratio STD was the ratio of the peak area of the sample to the peak area of the internal standard in the standard concentration solution.

### Test Example 9: Assay on In Vivo Pharmacokinetics of Compounds of Present Disclosure in Rats

By using SD rats as test animals, drug concentrations in plasma of the rats at different time points after intravenous injection and intragastric administration of the compounds of present disclosure were determined by LC/MS/MS. In vivo pharmacokinetic behaviors of the compounds of present disclosure in rats were studied to evaluate the pharmacokinetic characteristics of the compounds.

There were three healthy 6-8-week-old male SD rats in each group.

Intravenous injection: A certain amount of drug was weighed out, and then 10 vol % of N,N-dimethylacetamide, 33 vol % of triethylene glycol, and 57 vol % of physiological saline were added to prepare 1 mg/mL colorless clear and transparent liquid;

Intragastric administration: A certain amount of drug was weighed out, and then 0.5 wt % of hydroxypropyl methylcellulose, 0.1 vol % of tween 80, and 99.6 vol % of physiological saline were added to prepare 1 mg/mL white suspension.

After fasting overnight, SD rats were administered either intravenously or intragastrically.

The compounds of the present disclosure were injected intravenously to the rats, and then 0.2 mL of blood was collected from jugular veins at 0.083 h, 0.25 h, 0.5 h, 1 h, 2 h, 4 h, 8 h, and 24 h after the administration and placed in a test tube containing EDTA-K2. The test tube was then centrifuged at 4 °C and 4000 rpm for 5 min to separate plasma, and the plasma was stored at −75 °C.

Alternatively, the compounds of the present disclosure were administered intragastrically to the rats, and then 0.2 mL of blood was collected from jugular veins at 0.25 h, 0.5 h, 1 h, 2 h, 4 h, 8 h, and 24 h after the administration and placed in a test tube containing EDTA-K2. The test tube was then centrifuged at 4 °C and 3500 rpm for 10 min to separate plasma, and the plasma was stored at −75 °C.

Determination of the content of the test compounds in rat plasma after intravenous injection or intragastric administration of the compounds at different concentrations: 30 μL of rat plasma at each time point after administration was taken, 200 μL (50 ng/mL) of solution of internal standard dexamethasone in acetonitrile was added, and then the resulting mixture was vortexed for 30 s and centrifuged at 4 °C and 4700 rpm for 15 min. The supernatant of plasma samples was subjected to 3-fold dilution with water, and 2.0 μL of diluted solution was taken for LC-MS/MS analysis.

### Test Example 10: In Vitro Evaluation of Potential for PXR Activation in DPX2 Cells

DMSO solutions of the test compounds and the control compound rifampicin solution were prepared and diluted with Puracyp dosing medium (Puracyp, Cat. No. D-500-100) at 37 °C to respective test concentrations. The final concentrations of the test compounds were 30 μM, 10 μM, and 1 μM, and the final concentration of rifampicin was 20 μM. The final concentration of DMSO in the test solution was 0.1%. In addition, 0.1% DMSO solution prepared with Puracyp dosing medium was used as a control.

DPX2 cells were suspended in Puracyp Culture medium (Puracyp, Cat. No. D-500-100), and the cell density was 4.5×10⁵ cells/mL. The suspension was plated on a 96-well culture plate at 100 μL/well and then incubated at 37 °C for 24 h. The medium in appropriate wells of the 96-well culture plate was replaced (three times per well) with 100 μL of the test compound solution and a control compound solution. After replacement, incubation was performed at 37 °C for 24 h. The medium was again replaced (three times per well) with the newly prepared test compound solution and rifampicin test solution, and incubation was continued at 37 °C for 24 h.

The medium in the 96-well culture plate was pipetted out, and the plate was washed twice with PBS. 50 μL of reagent diluted as required by CellTiter-Fluor™ Cell Viability Assay Kit (Promega, Cat. No. G6082) was added, and the mixture was incubated at 37 °C for 0.5 h. The 96-well plate was cooled to room temperature, and the fluorescence value of each well at 505 nm was measured in a fluorescence mode with a microplate reader at an excitation wavelength of 400 nm. Then, 50 μL of reagent prepared as required by One-Glo Luciferase Assay System (Promega, Cat. No. E6120) was added to each well, and the mixture was incubated at room temperature for 5 min. The luminescence value of each well was read by using a lumen meter.

Normalized luciferase activity was determined by RLU/RFU, where RLU refers to the relative luminescence unit per test compound at each dose and RFU refers to the relative fluorescence unit per test compound at each dose. RLU and RFU of the samples were mean values of duplicate wells.

$$Fold of activation = \frac{{RLU}_{test} / {RFU}_{test}}{{RLU}_{vehicle} / {RFU}_{vehicle}}$$

The percentage of positive control was calculated as follows:

% Positive control=(fold activation_{test component}−1)/(fold activation_{positive control compound}−1)*100%
The Fold of activation was the fold of activation of PXR by the test sample;
RLUₛₐₘₚₗₑ was the relative luminescence intensity of the test sample;
RFUₛₐₘₚₗₑ was the relative fluorescence intensity of the test sample;
RLU_{vehicle} was the relative luminescence intensity of the blank control;
RFU_{vehicle} was the relative fluorescence intensity of the blank control;

% Positive control was the PXR activation rate of the test compound relative to the positive compound (rifampin).

1. A compound of formula (I) or a pharmaceutically acceptable salt thereof: 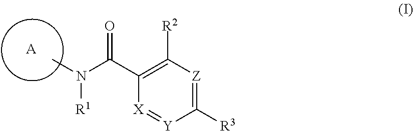
wherein
ring A is selected from 5-membered heteroaryl, pyridinonyl, azapyridinonyl, 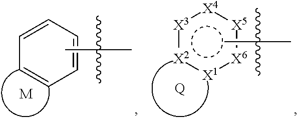
10-16 membered tricyclic heterocyclyl, 12-20 membered tetracyclic heterocyclyl, and 11-14 membered tricyclic heteroaryl;
the 5-membered heteroaryl is optionally substituted with one or more R^{1a}, and each R^{1a} is independently selected from halogen, CN, OH, NH₂, C₁-C₁₀ alkyl, C₂-C₁₀ alkenyl, C₂-C₁₀ alkynyl, C₃-C₁₀ cycloalkyl, C₅-C₁₀ cycloalkenyl, 4-10 membered heterocyclyl, C₆-C₁₀ aryl, and 5-10 membered heteroaryl, wherein the OH, NH₂, C₁-C₁₀ alkyl, C₂-C₁₀ alkenyl, C₂-C₁₀ alkynyl, C₃-C₁₀ cycloalkyl, C₅-C₁₀ cycloalkenyl, 4-10 membered heterocyclyl, C₆-C₁₀ aryl, or 5-10 membered heteroaryl is optionally substituted with one or more R^{1b}; when two R^{1a} are located at the ortho position of the 5-membered heteroaryl, the two R^{1a}, together with the atom to which they are attached, can also form C₅-C₇ monocyclic alkenyl, 4-7 membered monoheterocyclyl, phenyl, or 5-6 membered monoheteroaryl, wherein the C₅-C₇ monocyclic alkenyl, 4-7 membered monoheterocyclyl, phenyl, or 5-6 membered monoheteroaryl is optionally substituted with one or more R^{1b};
the pyridinonyl, azapyridinonyl, 10-16 membered tricyclic heterocyclyl, 12-20 membered tetracyclic heterocyclyl, or 11-14 membered tricyclic heteroaryl is optionally substituted with one or more R^{2b};
the ring M is selected from C₄-C₇ monocyclic alkenyl, 4-7 membered monoheterocyclyl, phenyl, and 5-6 membered monoheteroaryl; the structural unit 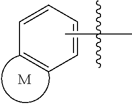
is substituted with one or more R^{3a}, wherein the R^{3a} is selected from C₃-C₁₀ cycloalkyl, C₅-C₁₀ cycloalkenyl, 4-10 membered heterocyclyl, C₆-C₁₀ aryl, and 5-10 membered heteroaryl; optionally, the 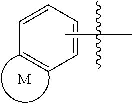
substituted with one or more R^{3a} can be further substituted with one or more R^{3b};
1, 2 or 3 of the X¹, X², X³, X⁴, X⁵, and X⁶ are selected from N and the others are selected from CR⁴, or 1 of the X³, X⁴, X⁵, and X⁶ is absent and the other 3, together with X¹ and X², form 5-membered heteroaryl, wherein the 5-membered heteroaryl is optionally substituted with one or more R^{4b};
each R⁴ is independently selected from being absent, H, and R^{4b};
the ring Q is selected from C₅-C₇ monocyclic alkenyl, 4-7 membered monoheterocyclyl, phenyl, and 5-6 membered monoheteroaryl, wherein the C₅-C₇ monocyclic alkenyl, 4-7 membered monoheterocyclyl, phenyl, or 5-6 membered monoheteroaryl is optionally substituted with one or more R^{4b};
R¹ is selected from H and C₁-C₆ alkyl, wherein the C₁-C₆ alkyl is optionally substituted with one or more halogen;
R² is selected from 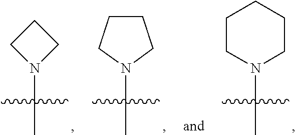
and the 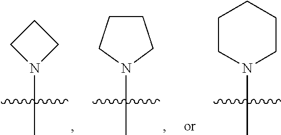
is optionally substituted with one or more R^{x}, wherein each R^{x} is independently selected from halogen, CN, OH, NH₂, C₁-C₆ alkyl, C₃-C₆ cycloalkyl, and 4-7 membered heterocyclyl, wherein the OH, NH₂, C₁-C₆ alkyl, C₃-C₆ cycloalkyl, or 4-7 membered heterocyclyl is optionally substituted with one or more R^{xb};
when the same carbon atom on 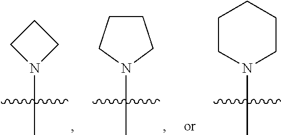
is simultaneously substituted with two R^{x}, the two R^{x}, together with the carbon atom, can also form C₃-C₁₀ cycloalkyl, C₃-C₁₀ cycloalkenyl, or 4-10 membered heterocyclyl, wherein the C₃-C₁₀ cycloalkyl, C₃-C₁₀ cycloalkenyl, or 4-10 membered heterocyclyl is optionally substituted with one or more R^{xb};
R³ is selected from L¹-L²-R^{y}, wherein L¹ is selected from —NR⁵—, —O—, —S—, —S(═O)—, —SO₂—, —C(═O)—, —NR⁵SO₂—, —NR⁵C(═O)—, —NR⁵—S(═O)(═NR⁵)—, —S(═O)(═NR⁵)—, —S═N—, 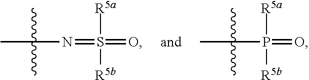
each R⁵ is independently selected from H and C₁-C₄ alkyl, each R^{5a} and R^{5b} is independently selected from C₁-C₄ alkyl, C₃-C₆ cycloalkyl, and 4-7 membered heterocyclyl, or the R^{5a} and R^{5b}, together with the atom to which they are attached, form 4-7 membered heterocyclyl; L² is selected from a chemical bond and C₁-C₄ alkylene; R^{y} is selected from H, C₃-C₆ cycloalkyl, and 4-7 membered heterocyclyl; each C₁-C₄ alkyl, C₁-C₄ alkylene, C₃-C₆ cycloalkyl, or 4-7 membered heterocyclyl described above is optionally substituted with one or more halogen, CN, OH, NH₂, O(C₁-C₄ alkyl), NH(C₁-C₄ alkyl), or N(C₁-C₄ alkyl)₂;
X, Y, and Z are each independently selected from N and CR⁶, wherein the R⁶ is selected from H, halogen, CN, OH, NH₂, C₁-C₆ alkyl, C₃-C₆ cycloalkyl, 4-7 membered heterocyclyl, O(C₁-C₄ alkyl), NH(C₁-C₄ alkyl), and N(C₁-C₄ alkyl)₂, wherein the C₁-C₆ alkyl, C₃-C₆ cycloalkyl, 4-7 membered heterocyclyl, O(C₁-C₄ alkyl), NH(C₁-C₄ alkyl), or N(C₁-C₄ alkyl)₂ is optionally substituted with one or more halogen or CN;
each R^{1b}, R^{2b}, R^{3b}, R^{4b}, and R^{xb} is independently selected from halogen, CN, OH, NH₂, C₁-C₆ alkyl, C₂-C₆ alkenyl, C₅-C₁₀ cycloalkenyl, C(O)R^{d}, C₃-C₆ cycloalkyl, 4-14 membered heterocyclyl, O(C₁-C₄ alkyl), NH(C₁-C₄ alkyl), and N(C₁-C₄ alkyl)₂, wherein the C₁-C₆ alkyl, C₂-C₆ alkenyl, C₅-C₁₀ cycloalkenyl, C₃-C₆ cycloalkyl, 4-14 membered heterocyclyl, O(C₁-C₄ alkyl), NH(C₁-C₄ alkyl), or N(C₁-C₄ alkyl)₂ is optionally substituted with one or more R^{c};
each R^{c} is independently selected from halogen, CN, OH, NH₂, C₁-C₆ alkyl, C₃-C₆ cycloalkyl, 4-7 membered heterocyclyl, O(C₁-C₄ alkyl), NH(C₁-C₄ alkyl), and N(C₁-C₄ alkyl)₂;
R^{d} is selected from OH, C₁-C₆ alkyl, O(C₁-C₄ alkyl), NH₂, NH(C₁-C₄ alkyl), and N(C₁-C₄ alkyl)₂.

2. The compound of formula (I) or the pharmaceutically acceptable salt thereof according to claim 1, wherein the azapyridinonyl, 10-16 membered tricyclic heterocyclyl, 12-20 membered tetracyclic heterocyclyl, or 11-14 membered tricyclic heteroaryl of ring A is selected from the following groups optionally substituted with one or more R^{2b}:

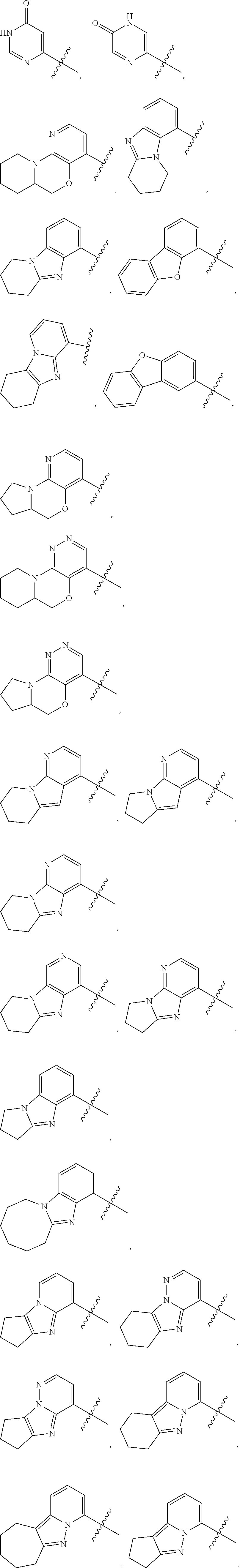

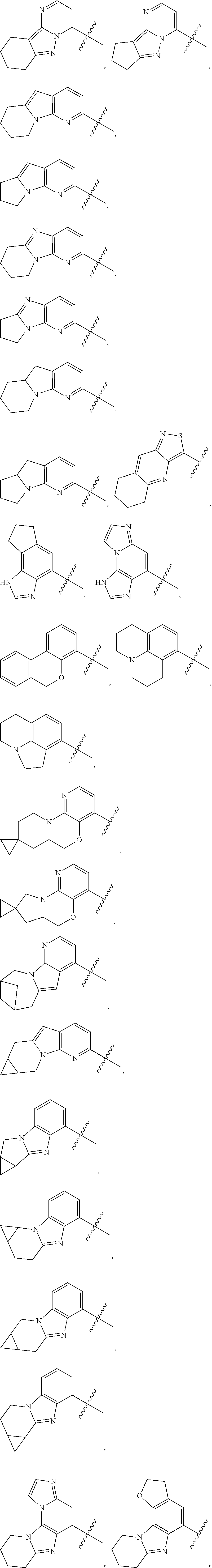

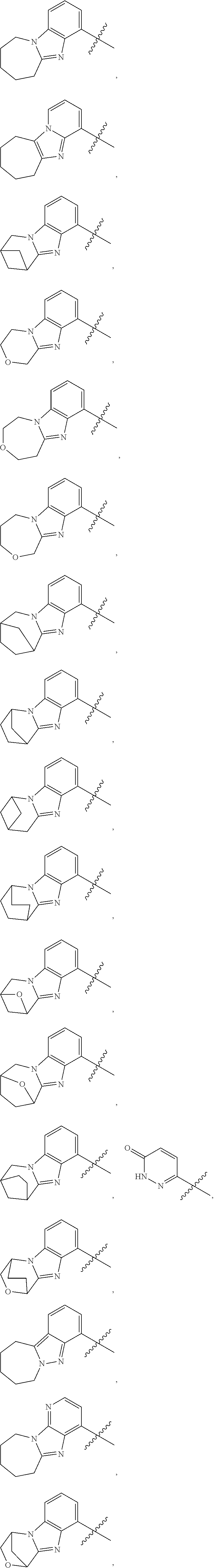

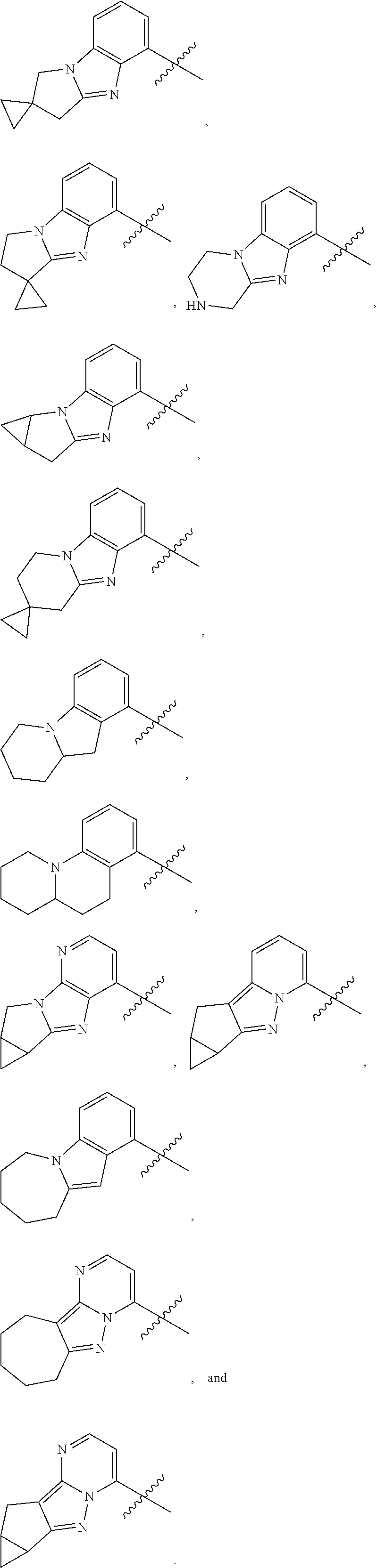

3. The compound of formula (I) or the pharmaceutically acceptable salt thereof according to claim 1, wherein the ring M in the structural unit 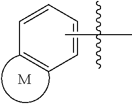
of ring A is selected from C₄-C₇ monocyclic alkenyl, 5-6 membered monoheteroaryl, and 4-7 membered monoheterocyclyl, and the structural unit 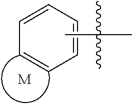
is substituted with one or more R^{3a};
optionally, the structural unit 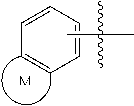
substituted with one or more R^{3a} can be further substituted with one or more R^{3b}; or
the structural unit 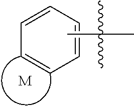
is selected from the following groups substituted with one or more R^{3a}: 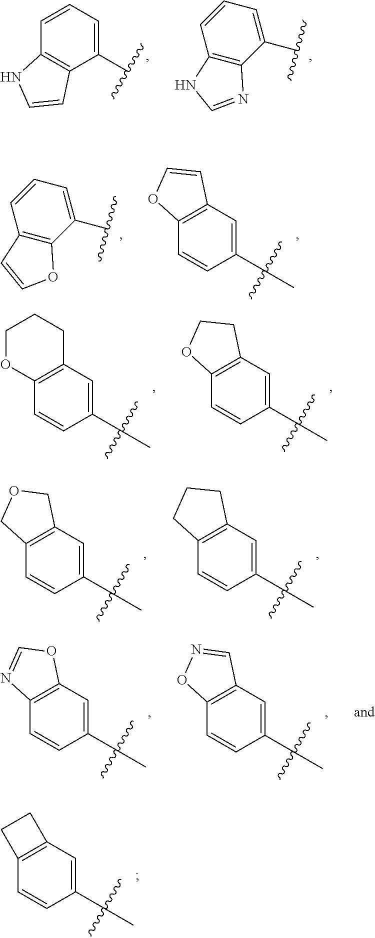
optionally, the aforementioned groups substituted with one or more R^{3a} can be further substituted with one or more R^{3b}.

4. The compound of formula (I) or the pharmaceutically acceptable salt thereof according to claim 1, wherein the structural unit 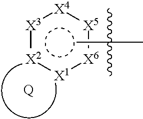
of ring A is selected from 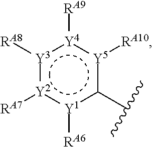
wherein 1, 2, or 3 of ring atoms Y¹, Y², Y³, Y⁴, and Y⁵ are N, and the others are C; any one of R^{A6} and R^{A8}, and R^{A7}, together with the atom to which they are attached, form C₅-C₇ monocyclic alkenyl, 4-7 membered monoheterocyclyl, phenyl, or 5-6 membered monoheteroaryl, wherein the C₅-C₇ monocyclic alkenyl, 4-7 membered monoheterocyclyl, phenyl, or 5-6 membered monoheteroaryl is optionally substituted with one or more R^{4b}; R^{A6}, R^{A8}, R^{A9}, and R^{A10} not involved in the ring forming are each independently selected from being absent, H, and R^{4b}; or
the structural unit 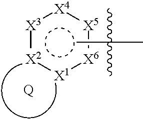
is selected from 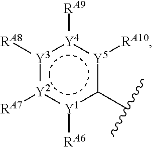
wherein 1 or 2 of ring atoms Y¹, Y², Y³, Y⁴, and Y⁵ are N, and the others are C; any one of R^{A6} and R^{A8}, and R^{A7}, together with the atom to which they are attached, form C₅ monocyclic alkenyl or 5-6 membered monoheteroaryl, wherein the C₅ monocyclic alkenyl or 5-6 membered monoheteroaryl is optionally substituted with one or more R^{4b}; R^{A6}, R^{A8}, R^{A9}, and R^{A10} not involved in the ring forming are each independently selected from being absent, H, and R^{4b}; or
the structural unit 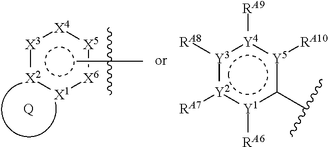
is selected from the following groups optionally substituted with one or more R^{4b}:

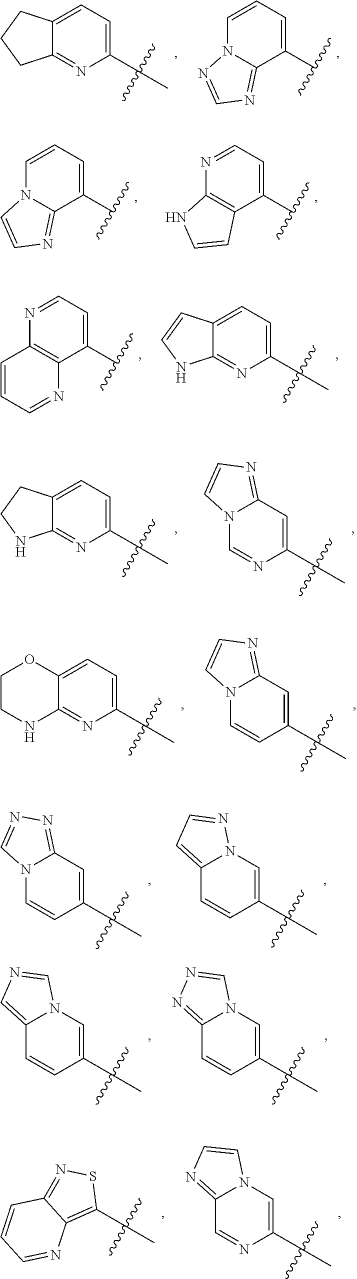

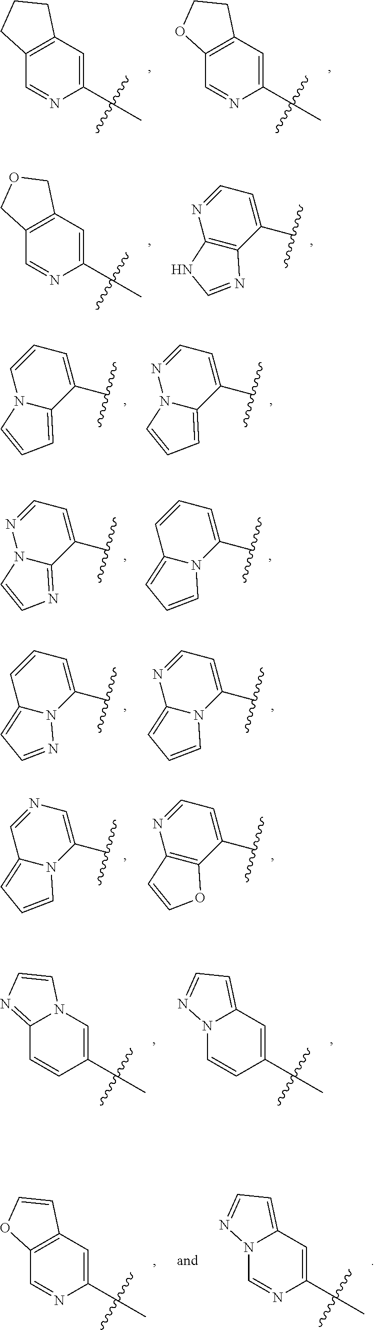

5. The compound of formula (I) or the pharmaceutically acceptable salt thereof according to claim 1, wherein R^{3a} is selected from C₃-C₆ cycloalkyl and 4-7 membered heterocyclyl, e.g., cyclobutyl, piperidinyl, morpholinyl, or azabicyclo[3.1.0]hexyl; or
R^{3a} is selected from C₃-C₆ cycloalkyl and 4-7 membered heterocyclyl, e.g., cyclobutyl, piperidinyl or morpholinyl; and/or
R^{3b} is selected from halogen and C₁-C₆ alkyl, e.g., F, Cl, and methyl;
optionally, R^{3b} is selected from halogen, e.g., F.

6. The compound of formula (I) or the pharmaceutically acceptable salt thereof according to claim 1, wherein
each R^{1b}, R^{2b}, R^{3b}, R^{4b}, and R^{xb} is independently selected from halogen, CN, OH, NH₂, C₁-C₆ alkyl, C₂-C₆ alkenyl, C₃-C₆ cycloalkyl, 4-14 membered heterocyclyl, O(C₁-C₄ alkyl), NH(C₁-C₄ alkyl), and N(C₁-C₄ alkyl)₂, wherein the C₁-C₆ alkyl, C₂-C₆ alkenyl, C₃-C₆ cycloalkyl, 4-14 membered heterocyclyl, O(C₁-C₄ alkyl), NH(C₁-C₄ alkyl), or N(C₁-C₄ alkyl)₂ is optionally substituted with one or more R^{c}; or
R^{2b} and/or R^{4b} are each independently selected from halogen, CN, C₁-C₆ alkyl, C₃-C₆ cycloalkyl, 4-10 membered heterocyclyl, C₂-C₄ alkenyl, C₅-C₁₀ cycloalkenyl, C(O)R^{d}, and O(C₁-C₄ alkyl), wherein the C₁-C₆ alkyl, C₃-C₆ cycloalkyl, 4-10 membered heterocyclyl, C₂-C₄ alkenyl, C₅-C₁₀ cycloalkenyl, or O(C₁-C₄ alkyl) is optionally substituted with one or more R^{c}, wherein the R^{c} is independently selected from halogen, CN, OH, NH₂, C₁-C₄ alkyl, and O(C₁-C₄ alkyl), and the R^{d} is independently selected from O(C₁-C₄ alkyl), NH₂, NH(C₁-C₄ alkyl), and N(C₁-C₄ alkyl)₂; or
R^{2b} and/or R^{4b} are each independently selected from halogen, CN, C₁-C₆ alkyl, C₃-C₆ cycloalkyl, 4-10 membered heterocyclyl, C₂-C₄ alkenyl, C₅-C₁₀ cycloalkenyl, C(O)R^{d}, and O(C₁-C₄ alkyl), wherein the C₁-C₆ alkyl, C₃-C₆ cycloalkyl, 4-10 membered heterocyclyl, C₂-C₄ alkenyl, C₅-C₁₀ cycloalkenyl, or O(C₁-C₄ alkyl) is optionally substituted with one or more R^{c}, wherein the R^{c} is independently selected from halogen and O(C₁-C₄ alkyl), and the R^{d} is independently selected from O(C₁-C₄ alkyl), NH₂, NH(C₁-C₄ alkyl), and N(C₁-C₄ alkyl)₂; or
R^{2b} and/or R^{4b} are each independently selected from halogen, CN, C₁-C₆ alkyl, C₃-C₆ cycloalkyl, 4-10 membered heterocyclyl, C₂-C₄ alkenyl, and O(C₁-C₄ alkyl), wherein the C₁-C₆ alkyl, C₃-C₆ cycloalkyl, 4-10 membered heterocyclyl, C₂-C₄ alkenyl, or O(C₁-C₄ alkyl) is optionally substituted with one or more R^{c}, wherein the R^{c} is independently selected from halogen, CN, OH, NH₂, and C₁-C₄ alkyl; or
R^{2b} is independently selected from halogen, CN, C₁-C₆ alkyl, C₃-C₆ cycloalkyl, 4-10 membered heterocyclyl, C₅-C₁₀ cycloalkenyl, C(O)R^{d}, and O(C₁-C₄ alkyl), wherein the C₁-C₆ alkyl, C₃-C₆ cycloalkyl, 4-10 membered heterocyclyl, C₅-C₁₀ cycloalkenyl, or O(C₁-C₄ alkyl) is optionally substituted with one or more R^{c}, and the R^{c} is selected from halogen and O(C₁-C₄ alkyl); or R^{2b} is independently selected from halogen, CN, C₁-C₆ alkyl, C₃-C₆ cycloalkyl, 4-10 membered heterocyclyl, C₅-C₁₀ cycloalkenyl, C(O)R^{d}, and O(C₁-C₄ alkyl), wherein the C₁-C₆ alkyl, 4-10 membered heterocyclyl, and C₅-C₁₀ cycloalkenyl are optionally substituted with one or more R^{c}, and the R^{c} is selected from halogen; or R^{2b} is selected from cyano, halogen, C₁-C₄ alkyl, O(C₁-C₄ alkyl), and piperidinyl, wherein the C₁-C₄ alkyl, O(C₁-C₄ alkyl), or piperidinyl is optionally substituted with one or more F, and/or R^{4b} is selected from C₁-C₄ alkyl, O(C₁-C₄ alkyl), 4-10 membered heterocyclyl, and C₂-C₄ alkenyl, wherein the C₁-C₄ alkyl, O(C₁-C₄ alkyl), 4-10 membered heterocyclyl, or C₂-C₄ alkenyl is optionally substituted with one or more F; or
R^{2b} is selected from CN F, methyl, trifluoromethyl, methoxy, methoxymethyl, cyclopropyl, 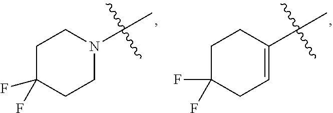 [SMILES: *N1CCC(F)(F)CC1 ; *C1=CCC(F)(F)CC1]
methoxycarbonyl, and ethylaminocarbonyl, or R^{2b} is selected from CN, F, methyl, trifluoromethyl, methoxy, cyclopropyl, 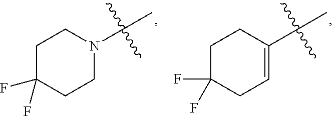 [SMILES: *N1CCC(F)(F)CC1 ; *C1=CCC(F)(F)CC1]
methoxycarbonyl, and ethylaminocarbonyl, or R^{2b} is selected from CN, F, methyl, methoxy, and 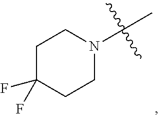 [SMILES: *N1CCC(F)(F)CC1]
and/or R^{4b} is selected from methyl, isopropyl, CF₃CH₂CH₂, CF₃CH₂CH₂O, 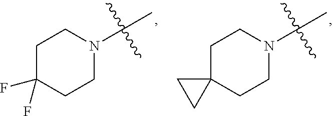 [SMILES: *N1CCC(F)(F)CC1 ; *N1CCC2(CC1)CC2]
and vinyl.

7. The compound of formula (I) or the pharmaceutically acceptable salt thereof according to claim 1, wherein ring A is selected from

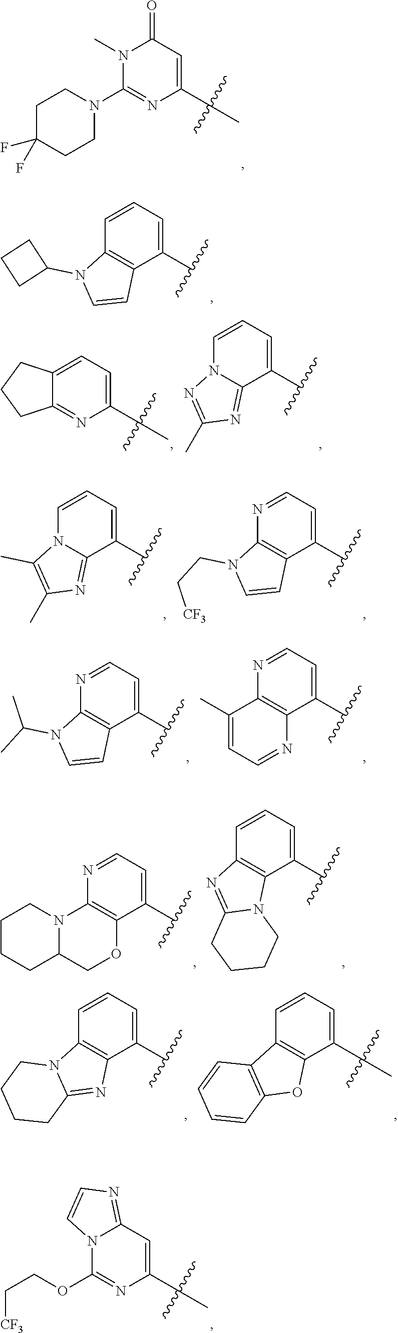

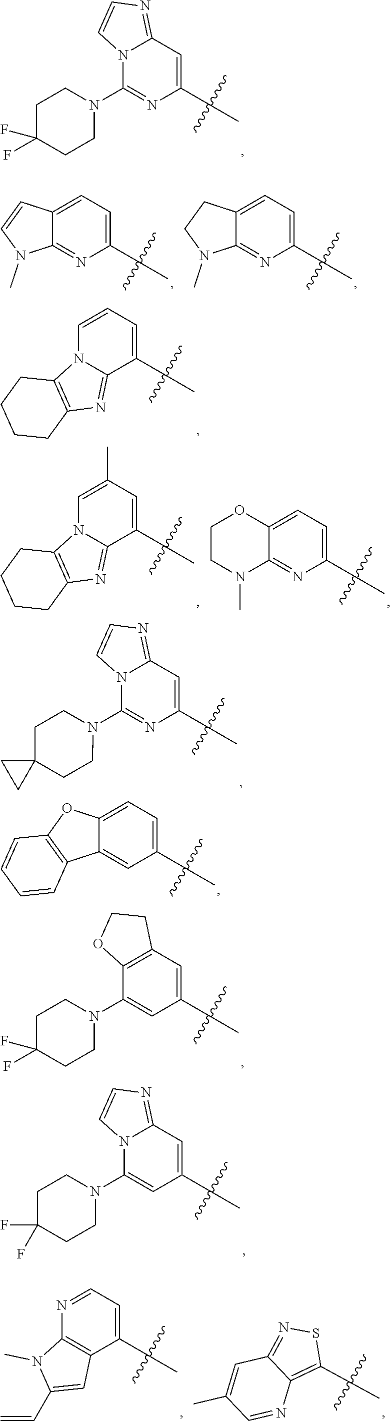

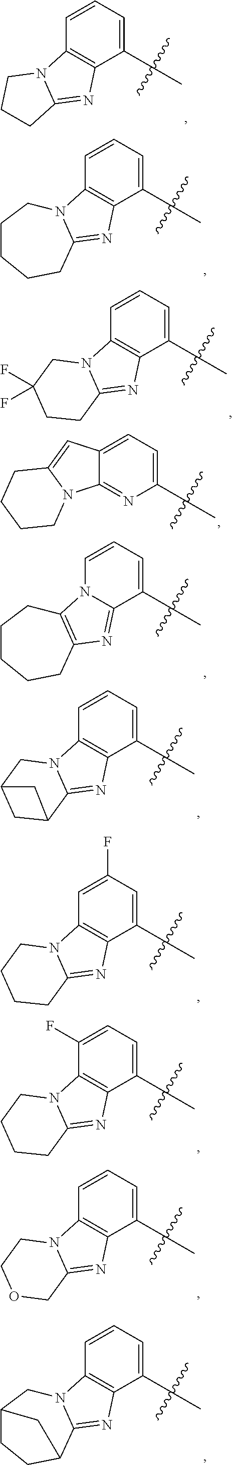

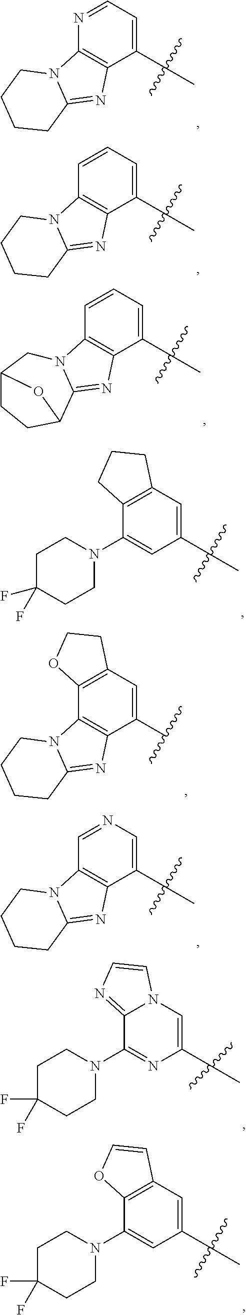

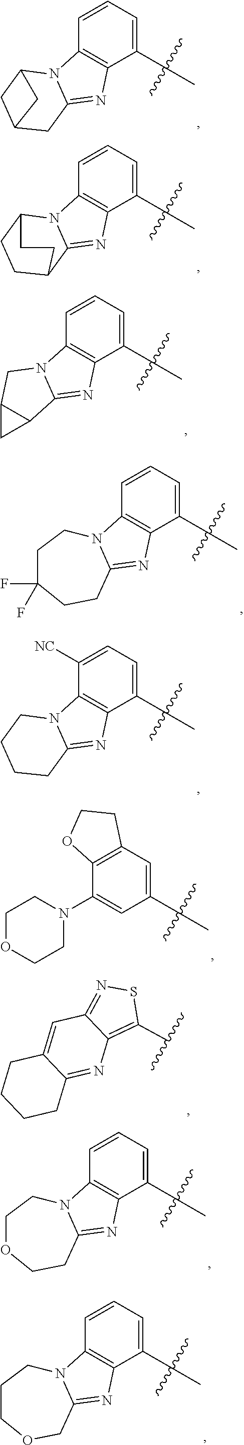

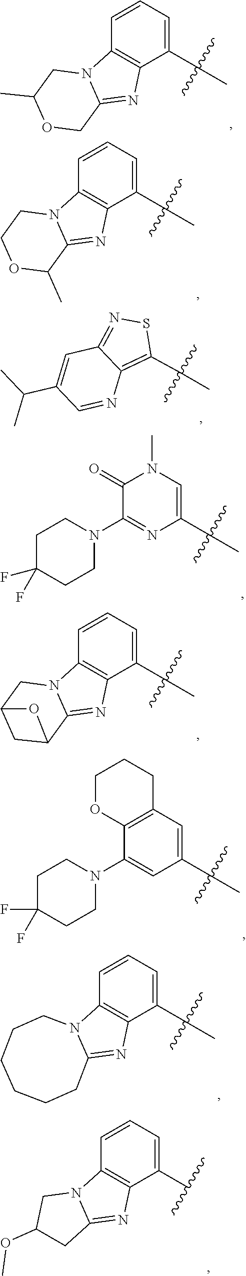

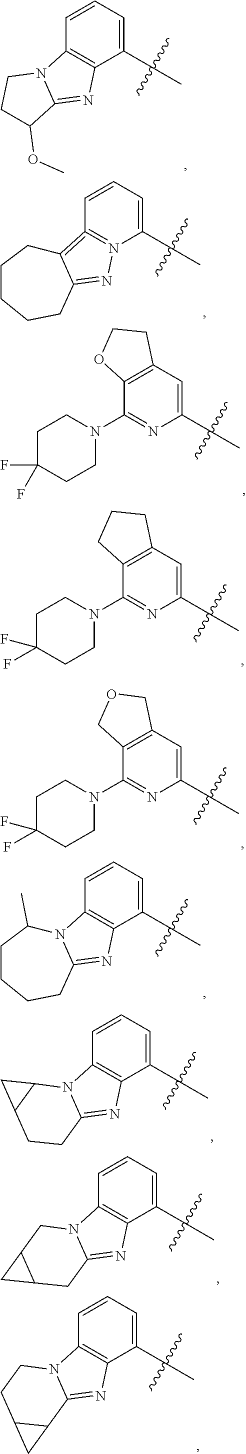

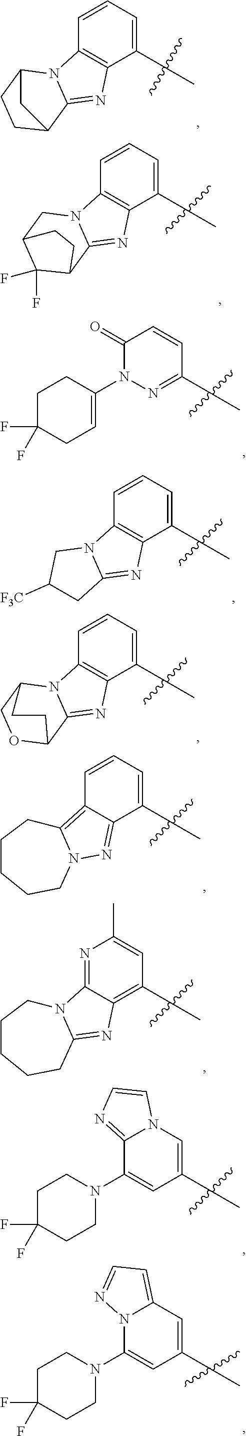

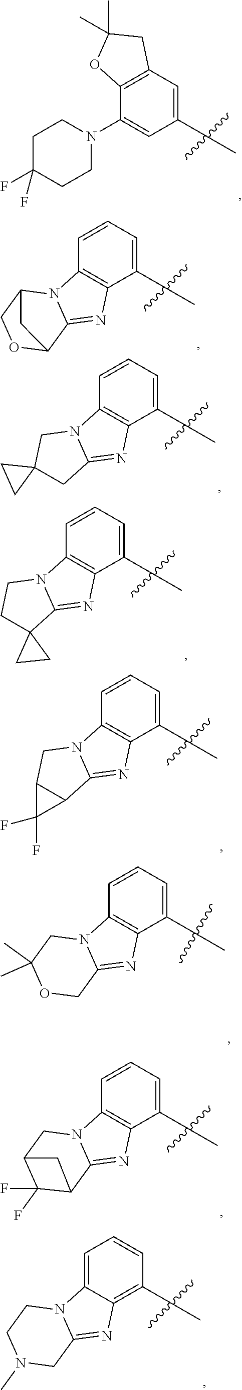

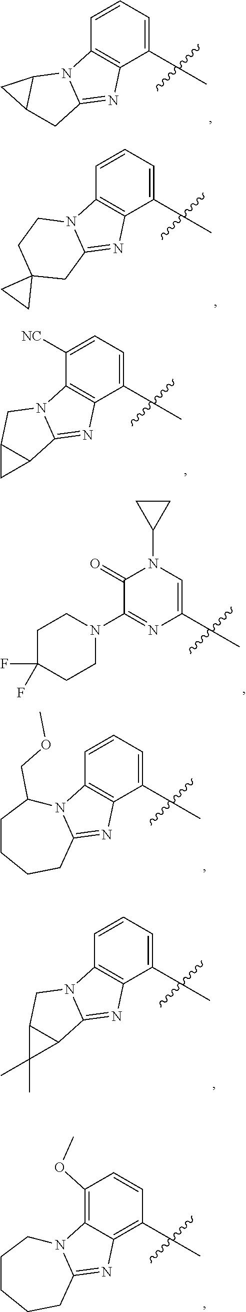

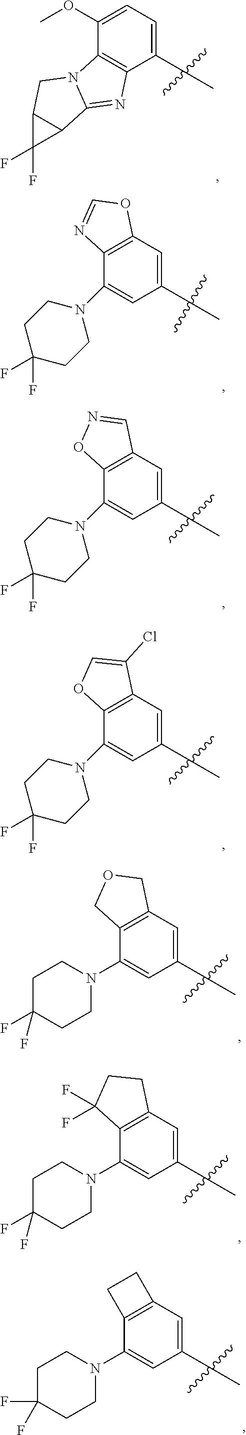

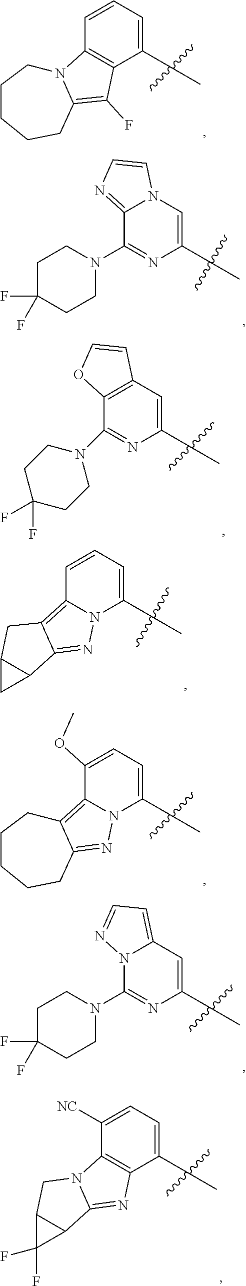

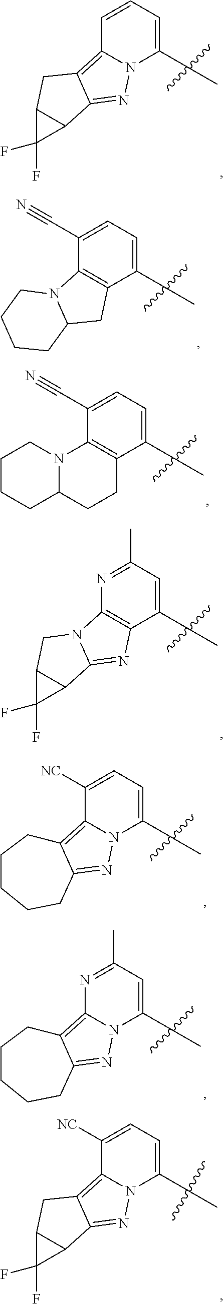

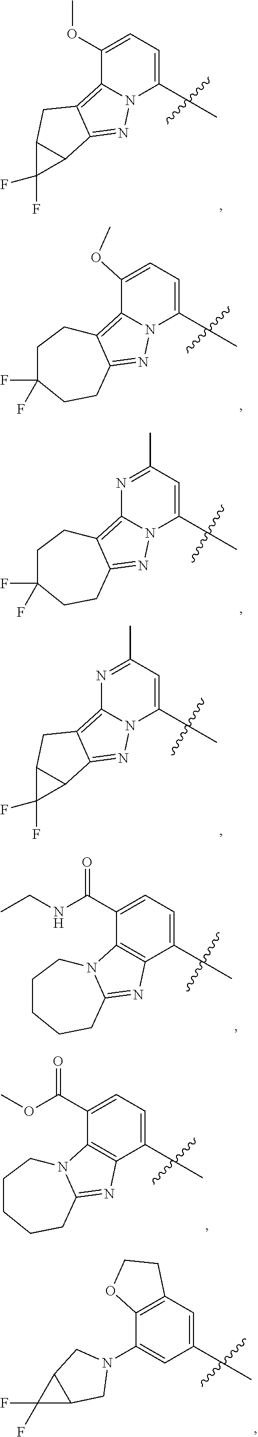

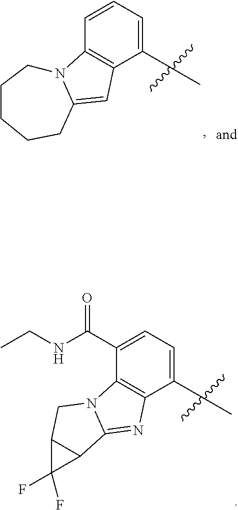 [SMILES: *c1cccc2c1cc1n2CCCCC1 ; *c1ccc(C(=O)NCC)c2c1nc1n2CC2C1C2(F)F]

8. The compound of formula (I) or the pharmaceutically acceptable salt thereof according to claim 1, wherein R¹ is selected from H and C₁-C₄ alkyl.

9. The compound of formula (I) or the pharmaceutically acceptable salt thereof according to claim 1, wherein R² is selected from 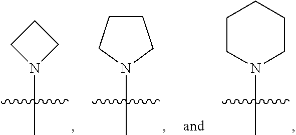
wherein the same carbon atom on the 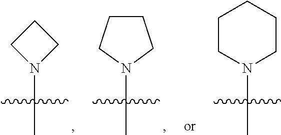
is simultaneously substituted with two R^{x}, and the two R^{x}, together with the carbon atom, form C₃-C₆ cycloalkyl, C₃-C₆ cycloalkenyl, or 4-7 membered heterocyclyl, wherein the C₃-C₆ cycloalkyl, C₃-C₆ cycloalkenyl, or 4-7 membered heterocyclyl is optionally substituted with one or more R^{xb}; or
R² is selected from 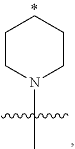 [SMILES: *N1CCCCC1]
the * carbon atom in the 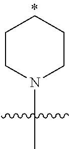 [SMILES: *N1CCCCC1]
is simultaneously substituted with two R^{x}, and the two R^{x}, together with the * carbon atom, form C₃-C₆ cycloalkyl, C₃-C₆ cycloalkenyl, or 4-7 membered heterocyclyl, wherein the C₃-C₆ cycloalkyl, C₃-C₆ cycloalkenyl, or 4-7 membered heterocyclyl is optionally substituted with one or more R^{xb}; or
R² is selected from 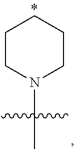 [SMILES: *N1CCCCC1]
the * carbon atom in the 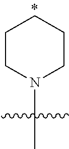 [SMILES: *N1CCCCC1]
is simultaneously substituted with two R^{x}, and the two R^{x}, together with the * carbon atom, form C₃-C₆ cycloalkyl, wherein the C₃-C₆ cycloalkyl is optionally substituted with one or more R^{xb}; or
R² is selected from

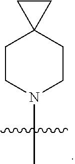 [SMILES: *N1CCC2(CC1)CC2]

10. The compound of formula (I) or the pharmaceutically acceptable salt thereof according to claim 1, wherein R³ is selected from L¹-L²-R^{y}, wherein Lⁱ is selected from —NH—, —O—, —S—, —S(═O)—, —SO₂—, —C(═O)—, —NHSO₂—, —NH(C═O)—, and 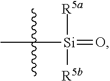
and the R^{5a} and R^{5b} are each independently selected from C₁-C₄ alkyl, or the R^{5a} and R^{5b}, together with the atom to which they are attached, form 4-7 membered heterocyclyl; L² is selected from a chemical bond and C₁-C₄ alkylene; R^{y} is selected from H, C₃-C₆ cycloalkyl, and 4-7 membered heterocyclyl; each C₁-C₄ alkyl, C₁-C₄ alkylene, C₃-C₆ cycloalkyl, or 4-7 membered heterocyclyl described above is optionally substituted with one or more halogen, CN, OH, or NH₂; or
R³ is selected from L¹-L²-R^{y}, wherein Lⁱ is selected from —NH—, —O—, —S—, —S(═O)—, —SO₂—, —C(═O)—, —NHSO₂—, and —NH(C═O)—, L² is selected from C₁-C₄ alkylene, and R^{y} is selected from H; the C₁-C₄ alkylene is optionally substituted with one or more halogen, CN, OH, or NH₂; or
R³ is selected from L¹-L²-R^{y}, wherein Lⁱ is selected from —NHSO₂—, L² is selected from C₁-C₄ alkylene, and R^{y} is selected from H, wherein the C₁-C₄ alkylene is optionally substituted with one or more halogen or OH; or R³ is selected from L¹-L²-R^{y}, wherein Lⁱ is selected from —NHSO₂—, L² is selected from C₁-C₄ alkylene, and R is selected from H, wherein the C₁-C₄ alkylene is optionally substituted with one or more OH;
R³ is selected from 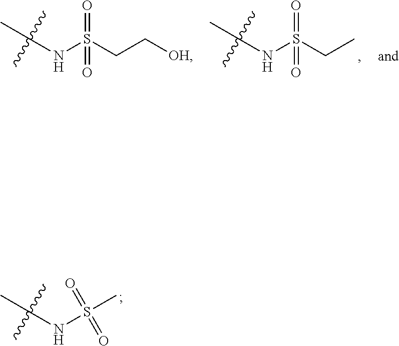
or
R³ is selected from

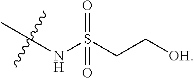 [SMILES: *NS(=O)(=O)CCO]

11. The compound of formula (I) or the pharmaceutically acceptable salt thereof according to claim 1, wherein X, Y, and Z are all selected from CH.

12. A compound or a pharmaceutically acceptable salt thereof, wherein the compound is selected from one of the following structures:

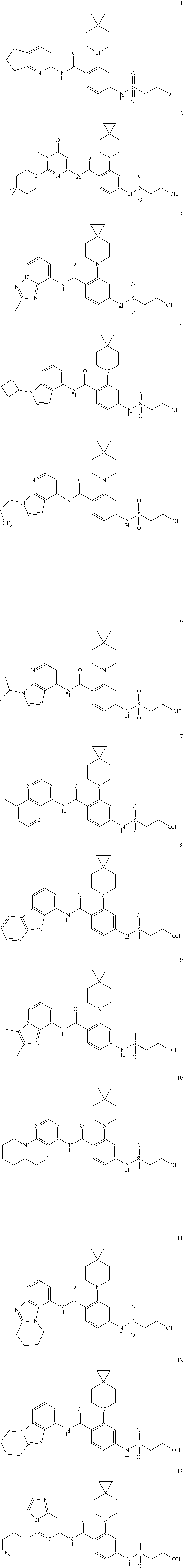

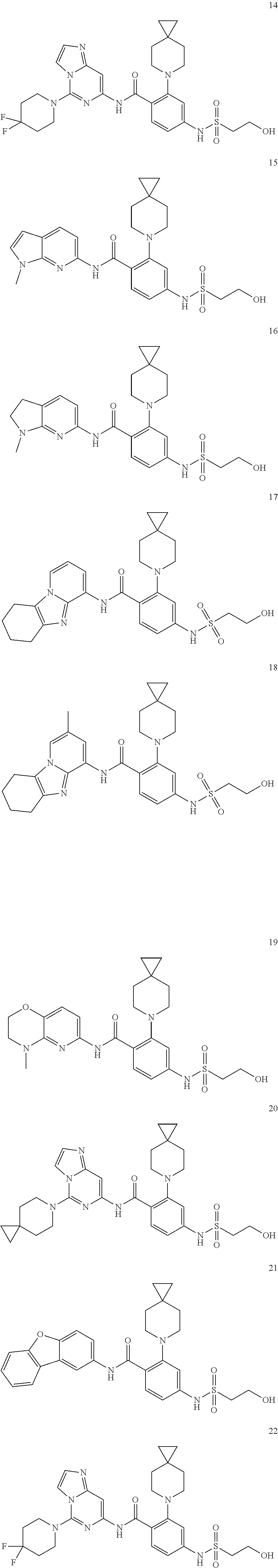

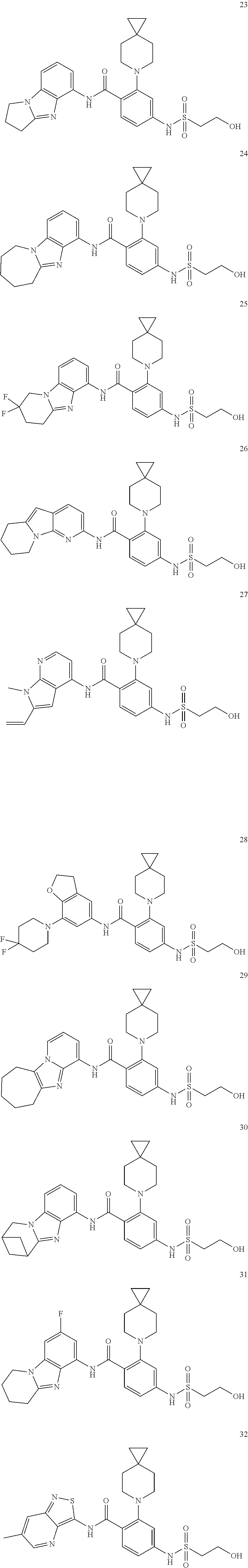

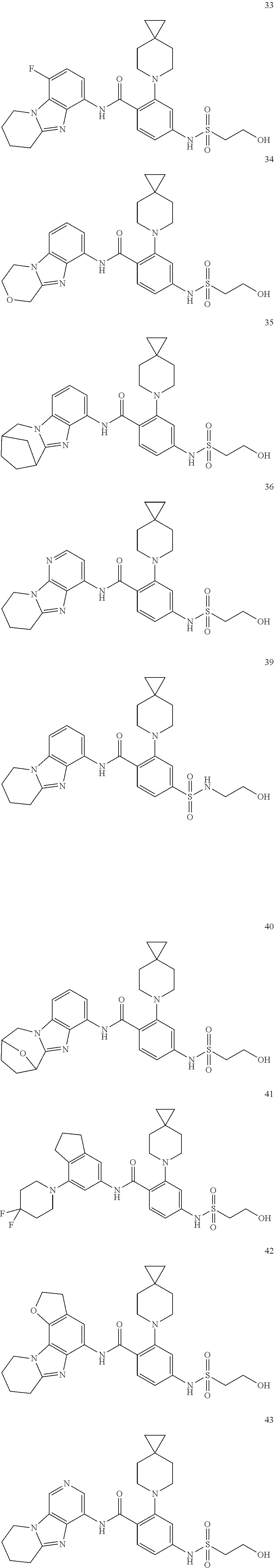

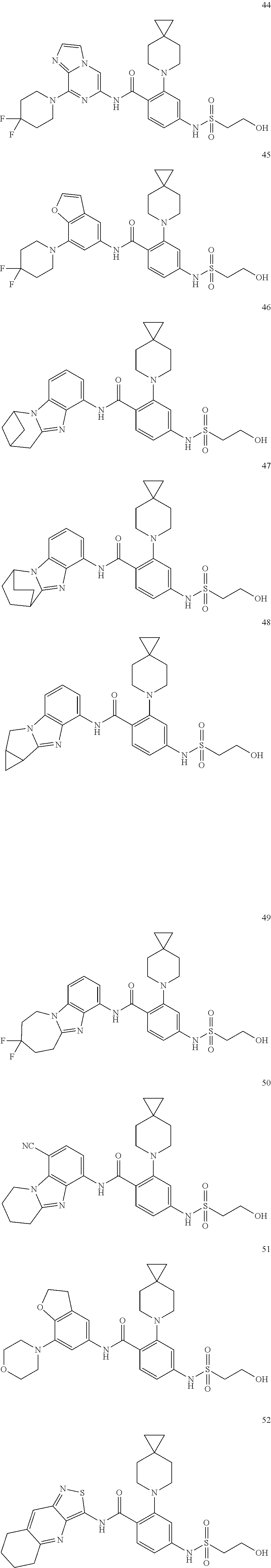

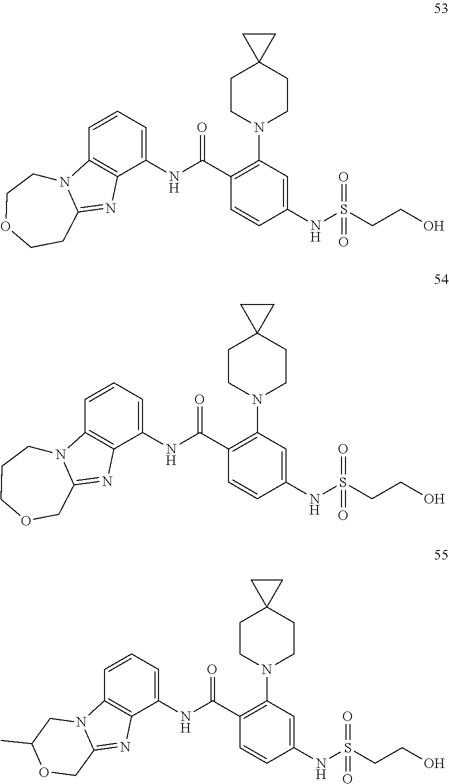 [SMILES: O=C(Nc1cccc2c1nc1n2CCOCC1)c1ccc(NS(=O)(=O)CCO)cc1N1CCC2(CC1)CC2 ; O=C(Nc1cccc2c1nc1n2CCCOC1)c1ccc(NS(=O)(=O)CCO)cc1N1CCC2(CC1)CC2 ; CC1Cn2c(nc3c(NC(=O)c4ccc(NS(=O)(=O)CCO)cc4N4CCC5(CC4)CC5)cccc32)CO1]

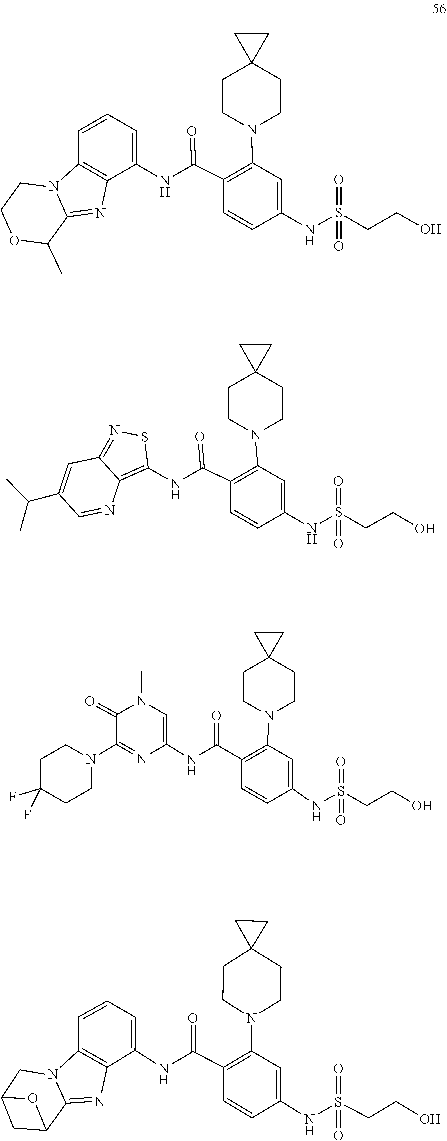 [SMILES: CC1OCCn2c1nc1c(NC(=O)c3ccc(NS(=O)(=O)CCO)cc3N3CCC4(CC3)CC4)cccc12 ; CC(C)c1cnc2c(NC(=O)c3ccc(NS(=O)(=O)CCO)cc3N3CCC4(CC3)CC4)snc2c1 ; Cn1cc(NC(=O)c2ccc(NS(=O)(=O)CCO)cc2N2CCC3(CC2)CC3)nc(N2CCC(F)(F)CC2)c1=O ; O=C(Nc1cccc2c1nc1n2CC2CC1O2)c1ccc(NS(=O)(=O)CCO)cc1N1CCC2(CC1)CC2]

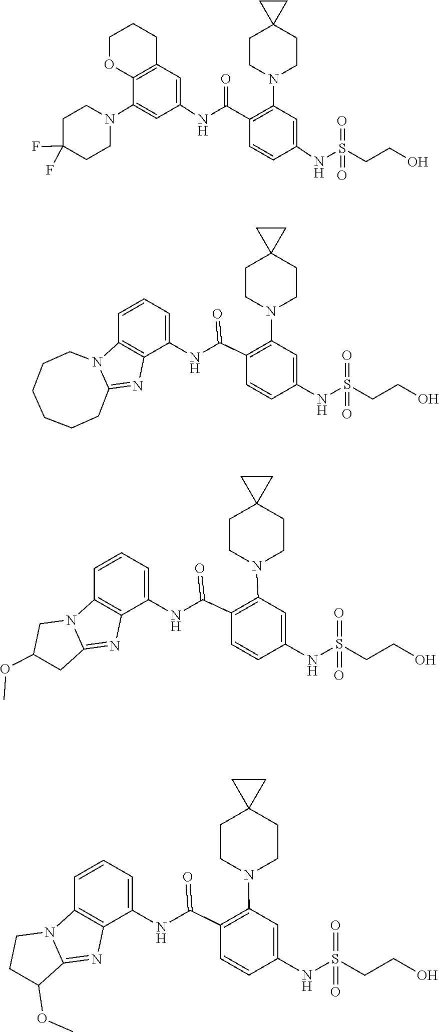 [SMILES: O=C(Nc1cc2c(c(N3CCC(F)(F)CC3)c1)OCCC2)c1ccc(NS(=O)(=O)CCO)cc1N1CCC2(CC1)CC2 ; O=C(Nc1cccc2c1nc1n2CCCCCC1)c1ccc(NS(=O)(=O)CCO)cc1N1CCC2(CC1)CC2 ; COC1Cc2nc3c(NC(=O)c4ccc(NS(=O)(=O)CCO)cc4N4CCC5(CC4)CC5)cccc3n2C1 ; COC1CCn2c1nc1c(NC(=O)c3ccc(NS(=O)(=O)CCO)cc3N3CCC4(CC3)CC4)cccc12]

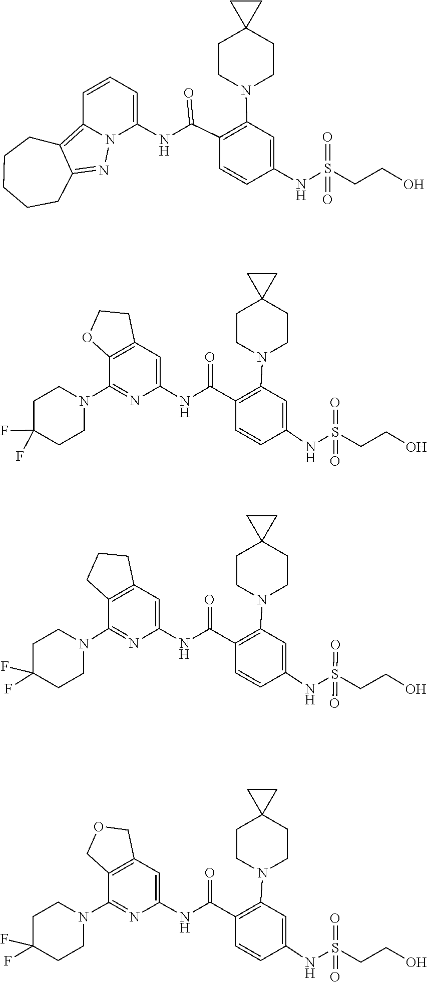 [SMILES: O=C(Nc1cccc2c3c(nn12)CCCCC3)c1ccc(NS(=O)(=O)CCO)cc1N1CCC2(CC1)CC2 ; O=C(Nc1cc2c(c(N3CCC(F)(F)CC3)n1)OCC2)c1ccc(NS(=O)(=O)CCO)cc1N1CCC2(CC1)CC2 ; O=C(Nc1cc2c(c(N3CCC(F)(F)CC3)n1)CCC2)c1ccc(NS(=O)(=O)CCO)cc1N1CCC2(CC1)CC2 ; O=C(Nc1cc2c(c(N3CCC(F)(F)CC3)n1)COC2)c1ccc(NS(=O)(=O)CCO)cc1N1CCC2(CC1)CC2]

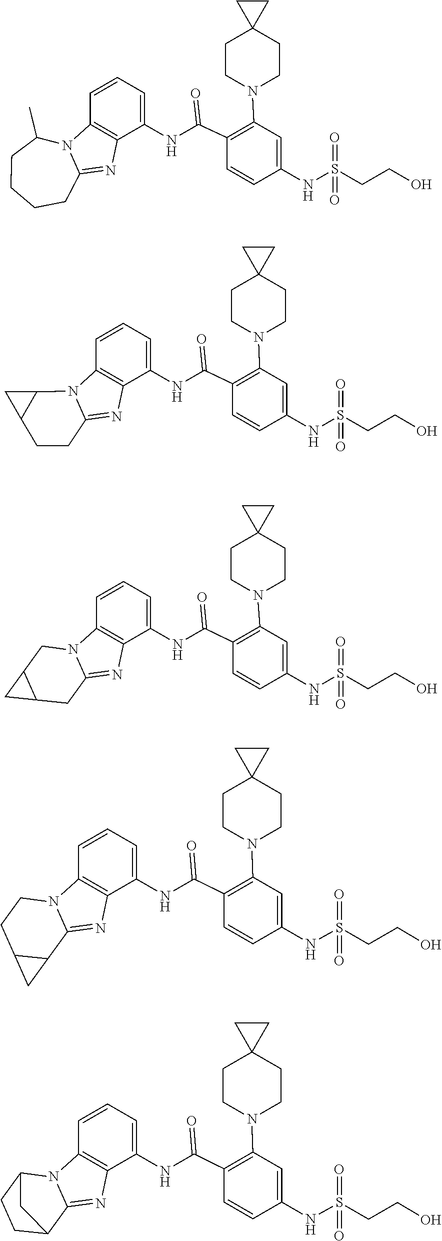

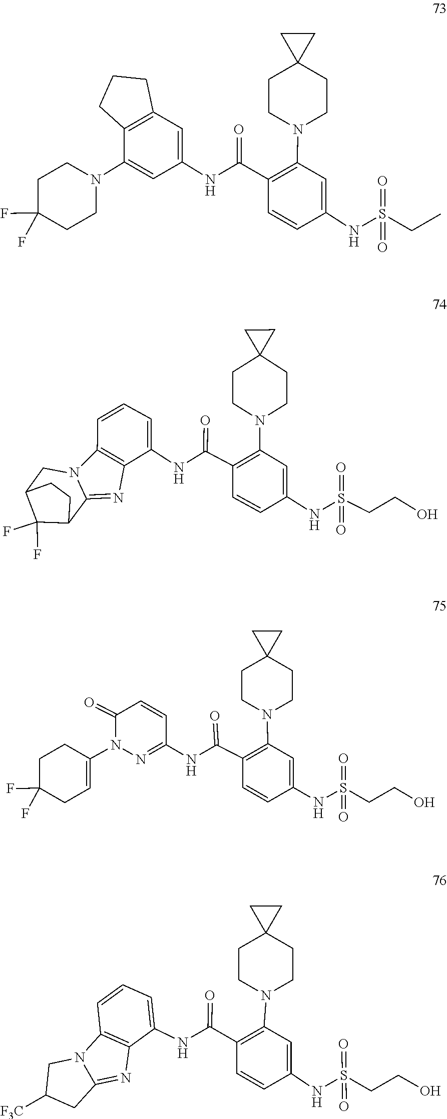 [SMILES: CCS(=O)(=O)Nc1ccc(C(=O)Nc2cc3c(c(N4CCC(F)(F)CC4)c2)CCC3)c(N2CCC3(CC2)CC3)c1 ; O=C(Nc1cccc2c1nc1n2CC2CCC1C2(F)F)c1ccc(NS(=O)(=O)CCO)cc1N1CCC2(CC1)CC2 ; O=C(Nc1ccc(=O)n(C2=CCC(F)(F)CC2)n1)c1ccc(NS(=O)(=O)CCO)cc1N1CCC2(CC1)CC2 ; O=C(Nc1cccc2c1nc1n2CC(C(F)(F)F)C1)c1ccc(NS(=O)(=O)CCO)cc1N1CCC2(CC1)CC2]

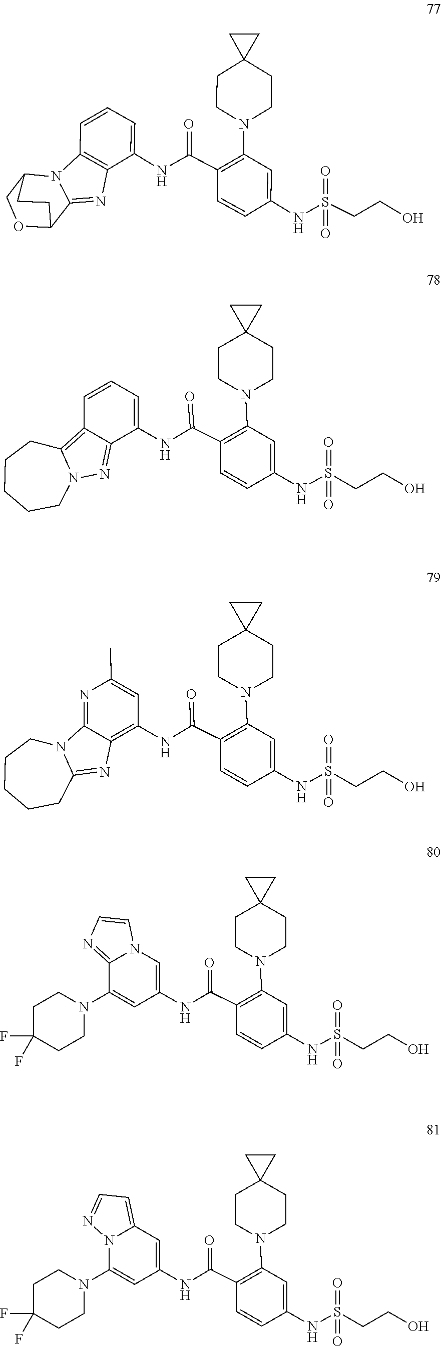 [SMILES: O=C(Nc1cccc2c1nc1n2C2CCC1OC2)c1ccc(NS(=O)(=O)CCO)cc1N1CCC2(CC1)CC2 ; O=C(Nc1cccc2c3n(nc12)CCCCC3)c1ccc(NS(=O)(=O)CCO)cc1N1CCC2(CC1)CC2 ; Cc1cc(NC(=O)c2ccc(NS(=O)(=O)CCO)cc2N2CCC3(CC2)CC3)c2nc3n(c2n1)CCCCC3 ; O=C(Nc1cc(N2CCC(F)(F)CC2)c2nccn2c1)c1ccc(NS(=O)(=O)CCO)cc1N1CCC2(CC1)CC2 ; O=C(Nc1cc(N2CCC(F)(F)CC2)n2nccc2c1)c1ccc(NS(=O)(=O)CCO)cc1N1CCC2(CC1)CC2]

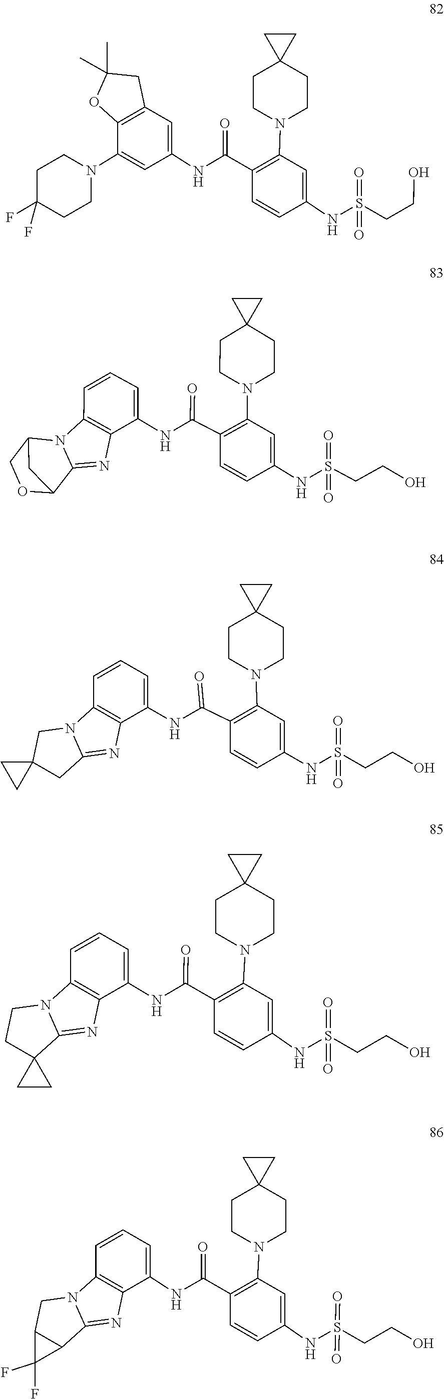 [SMILES: CC1(C)Cc2cc(NC(=O)c3ccc(NS(=O)(=O)CCO)cc3N3CCC4(CC3)CC4)cc(N3CCC(F)(F)CC3)c2O1 ; O=C(Nc1cccc2c1nc1n2C2COC1C2)c1ccc(NS(=O)(=O)CCO)cc1N1CCC2(CC1)CC2 ; O=C(Nc1cccc2c1nc1n2CC2(CC2)C1)c1ccc(NS(=O)(=O)CCO)cc1N1CCC2(CC1)CC2 ; O=C(Nc1cccc2c1nc1n2CCC12CC2)c1ccc(NS(=O)(=O)CCO)cc1N1CCC2(CC1)CC2 ; O=C(Nc1cccc2c1nc1n2CC2C1C2(F)F)c1ccc(NS(=O)(=O)CCO)cc1N1CCC2(CC1)CC2]

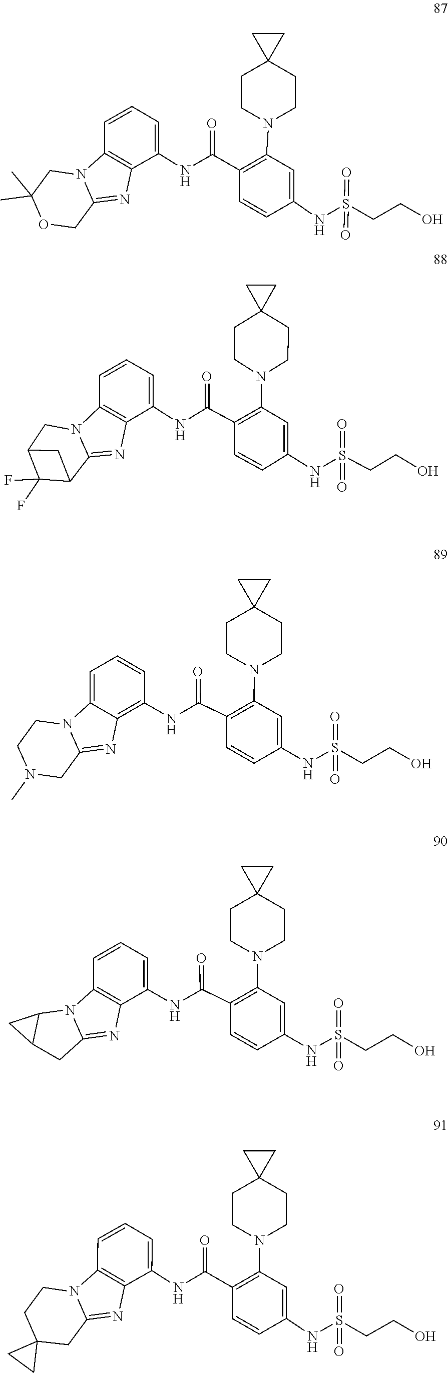

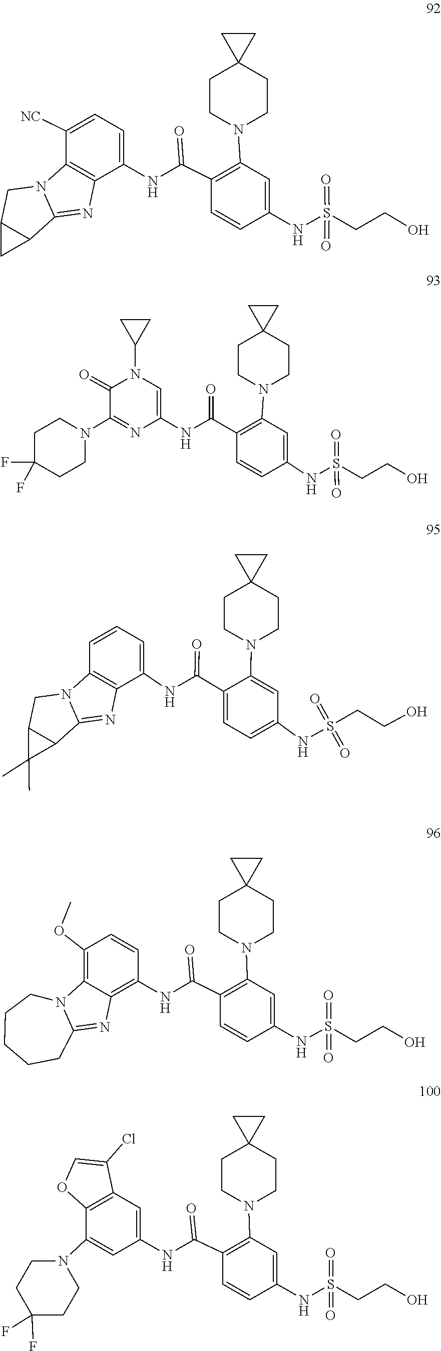 [SMILES: N#Cc1ccc(NC(=O)c2ccc(NS(=O)(=O)CCO)cc2N2CCC3(CC2)CC3)c2nc3n(c12)CC1CC31 ; O=C(Nc1cn(C2CC2)c(=O)c(N2CCC(F)(F)CC2)n1)c1ccc(NS(=O)(=O)CCO)cc1N1CCC2(CC1)CC2 ; CC1(C)C2Cn3c(nc4c(NC(=O)c5ccc(NS(=O)(=O)CCO)cc5N5CCC6(CC5)CC6)cccc43)C21 ; COc1ccc(NC(=O)c2ccc(NS(=O)(=O)CCO)cc2N2CCC3(CC2)CC3)c2nc3n(c12)CCCCC3 ; O=C(Nc1cc(N2CCC(F)(F)CC2)c2occ(Cl)c2c1)c1ccc(NS(=O)(=O)CCO)cc1N1CCC2(CC1)CC2]

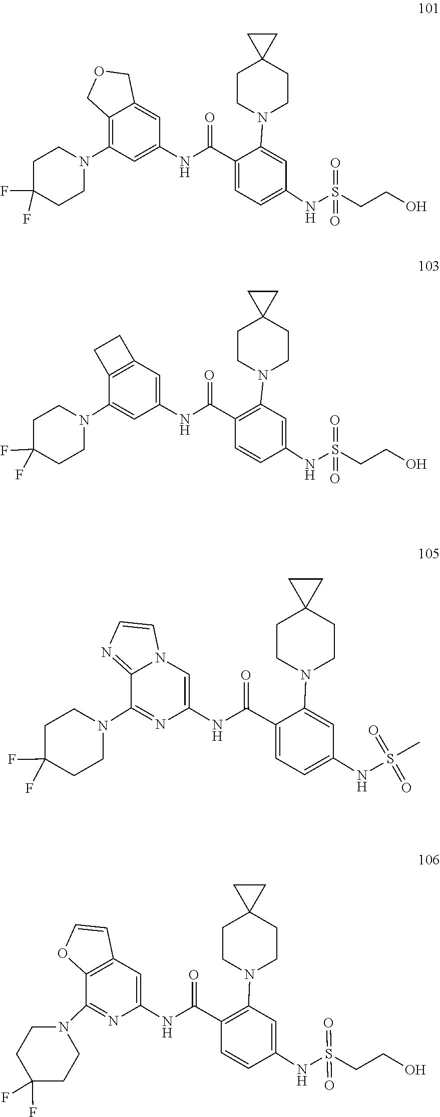 [SMILES: O=C(Nc1cc2c(c(N3CCC(F)(F)CC3)c1)COC2)c1ccc(NS(=O)(=O)CCO)cc1N1CCC2(CC1)CC2 ; O=C(Nc1cc2c(c(N3CCC(F)(F)CC3)c1)CC2)c1ccc(NS(=O)(=O)CCO)cc1N1CCC2(CC1)CC2 ; CS(=O)(=O)Nc1ccc(C(=O)Nc2cn3ccnc3c(N3CCC(F)(F)CC3)n2)c(N2CCC3(CC2)CC3)c1 ; O=C(Nc1cc2ccoc2c(N2CCC(F)(F)CC2)n1)c1ccc(NS(=O)(=O)CCO)cc1N1CCC2(CC1)CC2]

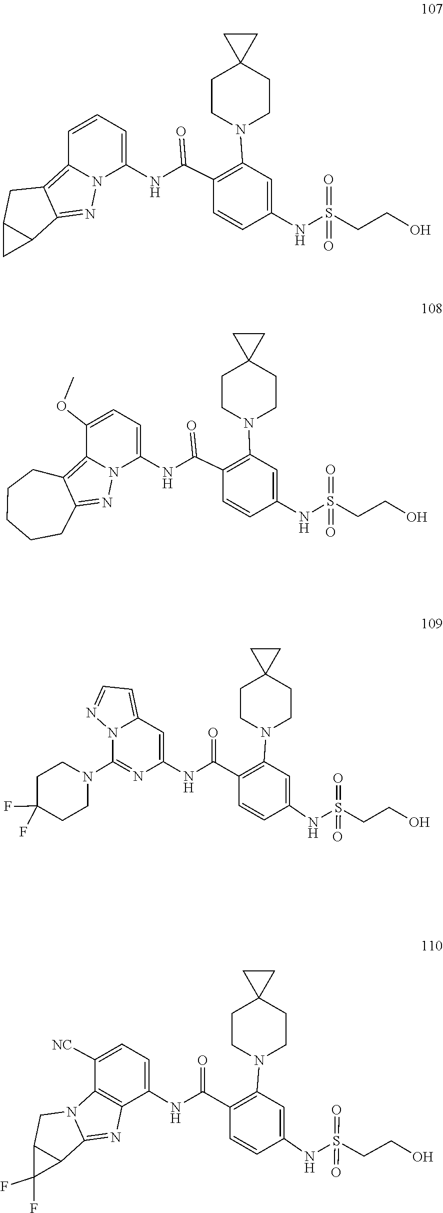 [SMILES: O=C(Nc1cccc2c3c(nn12)C1CC1C3)c1ccc(NS(=O)(=O)CCO)cc1N1CCC2(CC1)CC2 ; COc1ccc(NC(=O)c2ccc(NS(=O)(=O)CCO)cc2N2CCC3(CC2)CC3)n2nc3c(c12)CCCCC3 ; O=C(Nc1cc2ccnn2c(N2CCC(F)(F)CC2)n1)c1ccc(NS(=O)(=O)CCO)cc1N1CCC2(CC1)CC2 ; N#Cc1ccc(NC(=O)c2ccc(NS(=O)(=O)CCO)cc2N2CCC3(CC2)CC3)c2nc3n(c12)CC1C3C1(F)F]

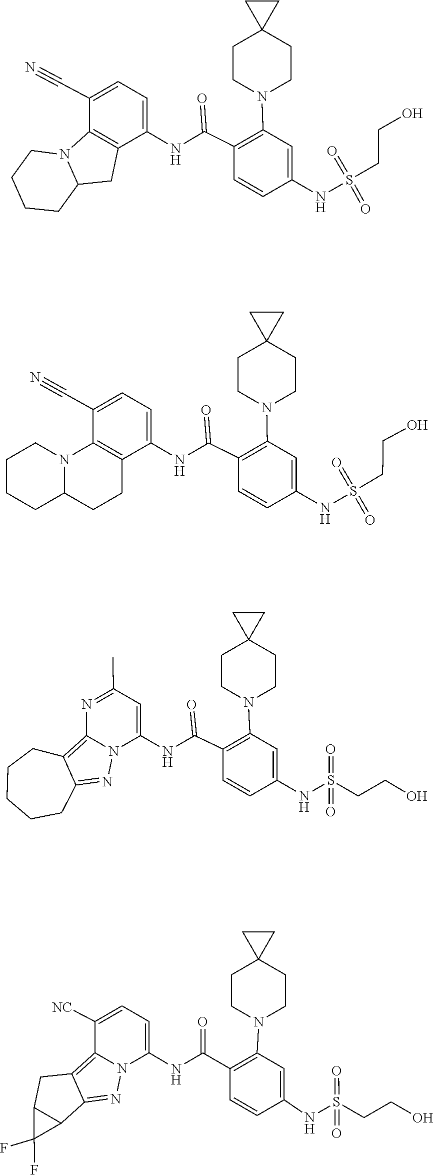 [SMILES: N#Cc1ccc(NC(=O)c2ccc(NS(=O)(=O)CCO)cc2N2CCC3(CC2)CC3)c2c1N1CCCCC1C2 ; N#Cc1ccc(NC(=O)c2ccc(NS(=O)(=O)CCO)cc2N2CCC3(CC2)CC3)c2c1N1CCCCC1CC2 ; Cc1cc(NC(=O)c2ccc(NS(=O)(=O)CCO)cc2N2CCC3(CC2)CC3)n2nc3c(c2n1)CCCCC3 ; N#Cc1ccc(NC(=O)c2ccc(NS(=O)(=O)CCO)cc2N2CCC3(CC2)CC3)n2nc3c(c12)CC1C3C1(F)F]

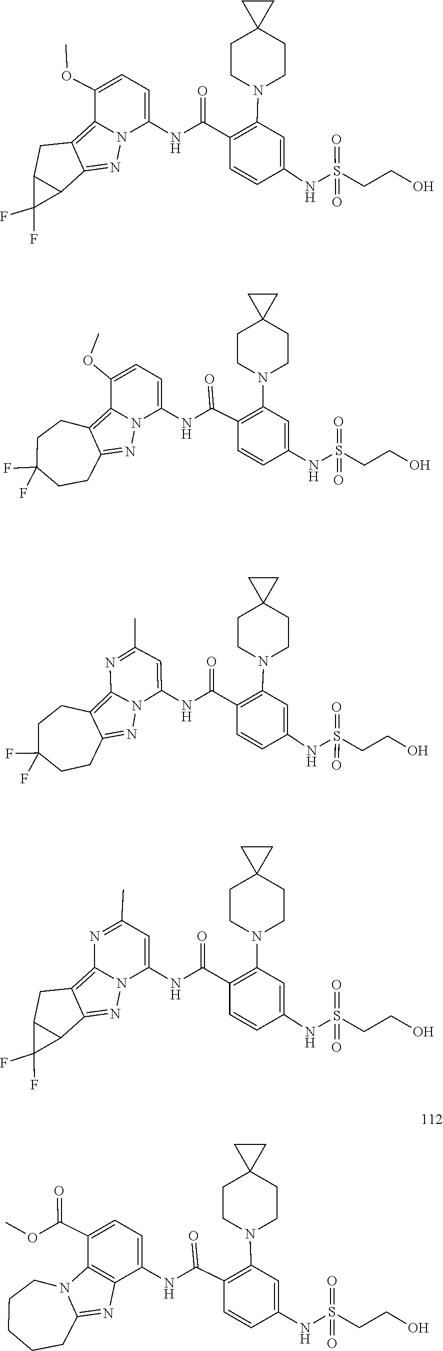 [SMILES: COc1ccc(NC(=O)c2ccc(NS(=O)(=O)CCO)cc2N2CCC3(CC2)CC3)n2nc3c(c12)CC1C3C1(F)F ; COc1ccc(NC(=O)c2ccc(NS(=O)(=O)CCO)cc2N2CCC3(CC2)CC3)n2nc3c(c12)CCC(F)(F)CC3 ; Cc1cc(NC(=O)c2ccc(NS(=O)(=O)CCO)cc2N2CCC3(CC2)CC3)n2nc3c(c2n1)CCC(F)(F)CC3 ; Cc1cc(NC(=O)c2ccc(NS(=O)(=O)CCO)cc2N2CCC3(CC2)CC3)n2nc3c(c2n1)CC1C3C1(F)F ; COC(=O)c1ccc(NC(=O)c2ccc(NS(=O)(=O)CCO)cc2N2CCC3(CC2)CC3)c2nc3n(c12)CCCCC3]

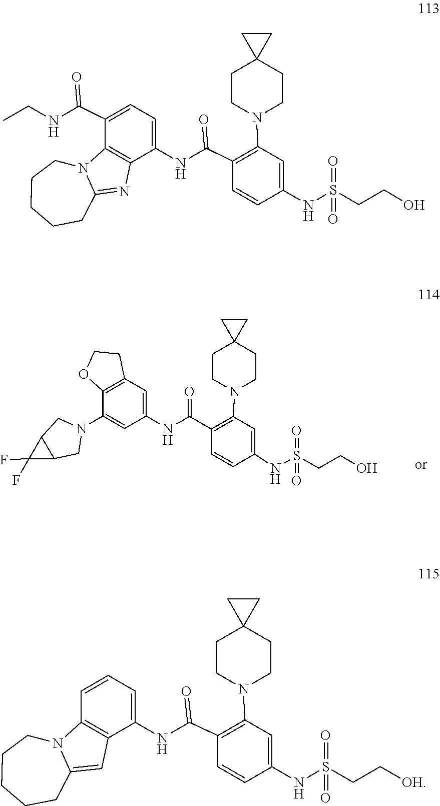 [SMILES: CCNC(=O)c1ccc(NC(=O)c2ccc(NS(=O)(=O)CCO)cc2N2CCC3(CC2)CC3)c2nc3n(c12)CCCCC3 ; O=C(Nc1cc2c(c(N3CC4C(C3)C4(F)F)c1)OCC2)c1ccc(NS(=O)(=O)CCO)cc1N1CCC2(CC1)CC2 ; O=C(Nc1cccc2c1cc1n2CCCCC1)c1ccc(NS(=O)(=O)CCO)cc1N1CCC2(CC1)CC2]

13. A pharmaceutical composition, comprising the compound or the pharmaceutically acceptable salt thereof according to claim 1 and a pharmaceutically acceptable excipient.

14. A method for treating a KIF18A-related disease, comprising administering to a patient a therapeutically effective amount of the compound or the pharmaceutically acceptable salt thereof according to claim.

15. The method according to claim 14, wherein the KIF18A-related disease is a tumor or an ovarian cancer.

16. (canceled)

17. A method for treating a KIF18A-related disease, comprising administering to a patient a therapeutically effective amount of the pharmaceutical composition according to claim 13.

18. The method according to claim 17, wherein the KIF18A-related disease is a tumor or an ovarian cancer.

19. (canceled)

20. A compound of formula (II) or a pharmaceutically acceptable salt thereof: 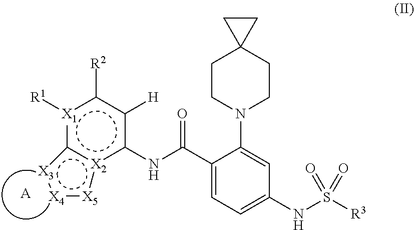
wherein
A is a monocyclic or bicyclic ring selected from 4-10 membered carbon ring and 4-10 membered heterocycle, wherein the 4-10 membered carbon ring or 4-10 membered heterocycle is optionally substituted with one or more R^{b}, and each R^{b} is independently selected from halogen, OH, C₁-C₆ alkyl, O(C₁-C₄ alkyl), NH₂, NH(C₁-C₄ alkyl), and N(C₁-C₄ alkyl)₂, wherein the C₁-C₆ alkyl is optionally substituted with one or more groups independently selected from halogen, cyano, OH, NH₂, and O(C₁-C₄ alkyl);
X₁, X₂, X₃, X₄, and X₅ are each independently CR^{a} or nitrogen, wherein R^{a} are each independently selected from being absent, H, and halogen;
R¹ is absent, H, C₁-C₆ alkyl, cyano, C₁-C₆ alkoxy, (CH₂)ᵣ—C(O)R^{d}, halogen, (CH₂)ᵣ—C₃-C₁₀ cycloalkyl, (CH₂)ᵣ—C₅-C₁₀ cycloalkenyl, (CH₂)ᵣ-4-10 membered heterocyclyl, (CH₂)ᵣ—C₆-C₁₀ aryl, or (CH₂)ᵣ-5-10 membered heteroaryl, wherein r is 0, 1, 2, or 3, and the C₁-C₆ alkyl, C₁-C₆ alkoxy, C₃-C₁₀ cycloalkyl, C₅-C₁₀ cycloalkenyl, 4-10 membered heterocyclyl, C₆-C₁₀ aryl, or 5-10 membered heteroaryl is optionally substituted with one or more groups independently selected from halogen, CN, OH, NH₂, C₁-C₆ alkyl, C₃-C₆ cycloalkyl, 4-7 membered heterocyclyl, O(C₁-C₄ alkyl), NH(C₁-C₄ alkyl), and N(C₁-C₄ alkyl)₂;
R² is H, C₁-C₆ alkyl, cyano, C₁-C₆ alkoxy, C(O)R^{d}, halogen, C₁-C₆ haloalkyl, or C₁-C₆ haloalkoxy; R^{d} is selected from OH, C₁-C₆ alkyl, O(C₁-C₄ alkyl), NH₂, NH(C₁-C₄ alkyl), and N(C₁-C₄ alkyl)₂; and
R³ is selected from C₁-C₆ alkyl optionally substituted with one or more halogen or OH.

21. The compound of formula (II) or the pharmaceutically acceptable salt thereof according to claim 20, wherein the structural unit 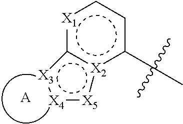
is selected from the following groups optionally substituted with one or more R^{b}: 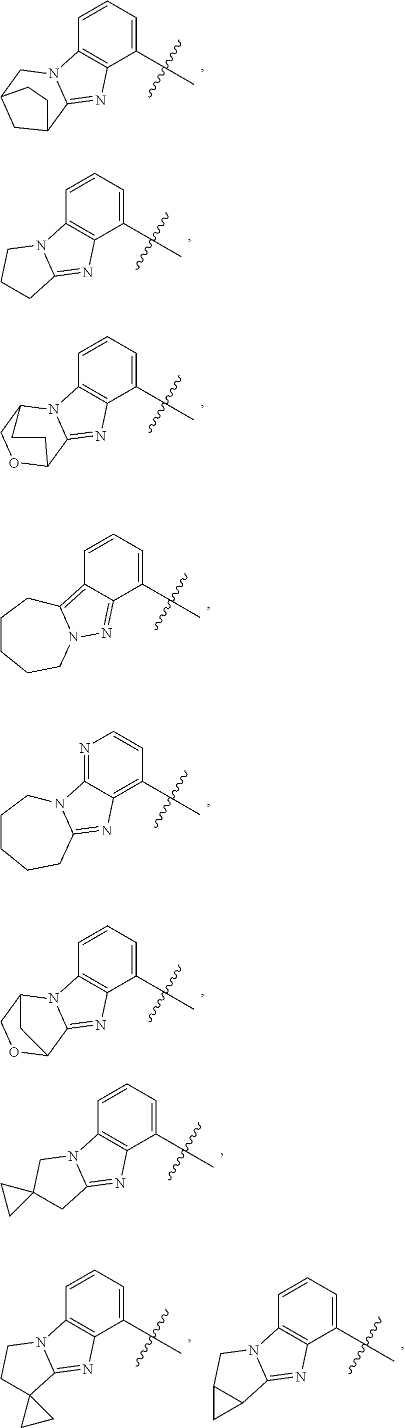 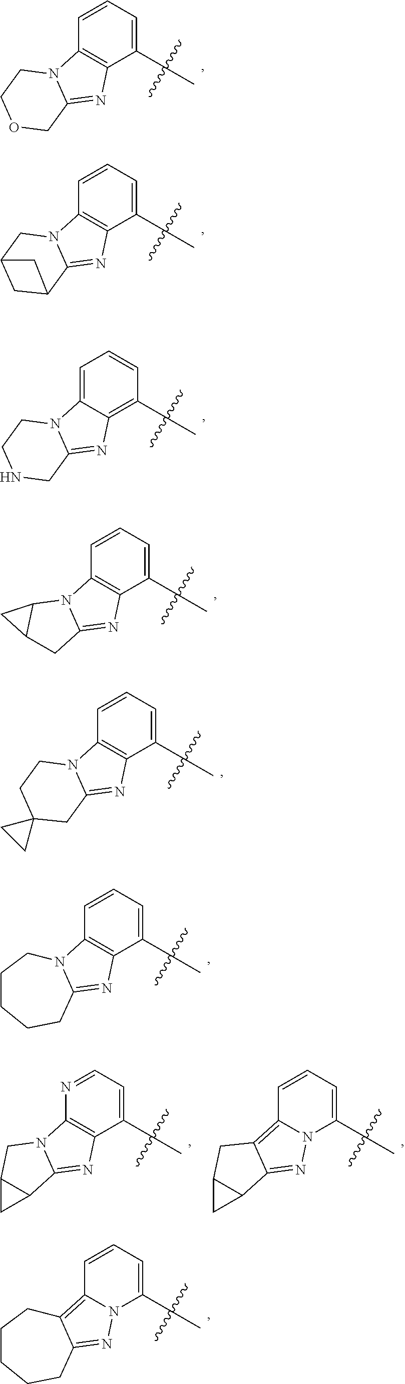 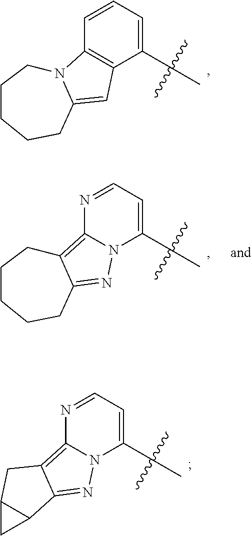 [SMILES: *c1cccc2c1cc1n2CCCCC1 ; *c1ccnc2c3c(nn12)CCCCC3 ; *c1ccnc2c3c(nn12)C1CC1C3]
or
the structural unit 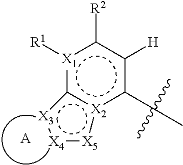
is selected from

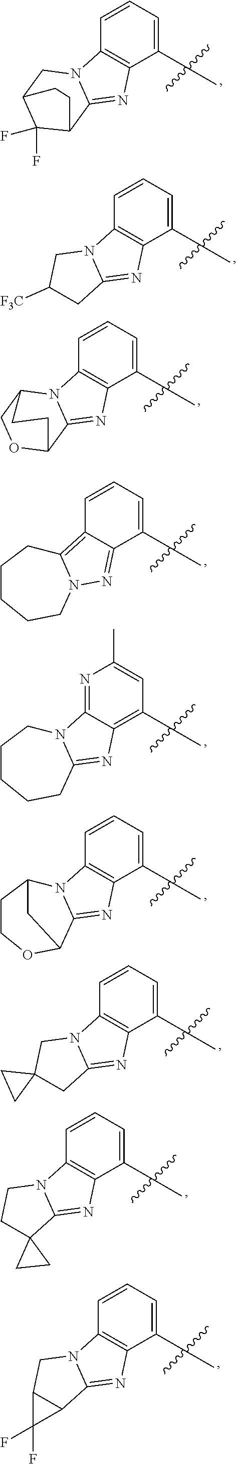

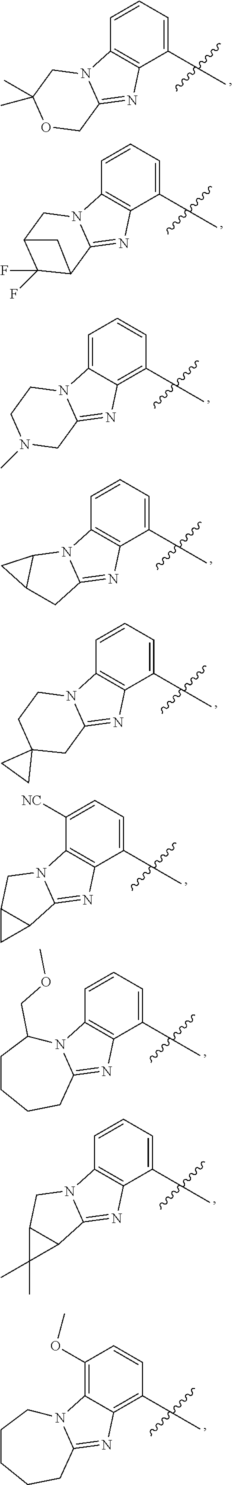

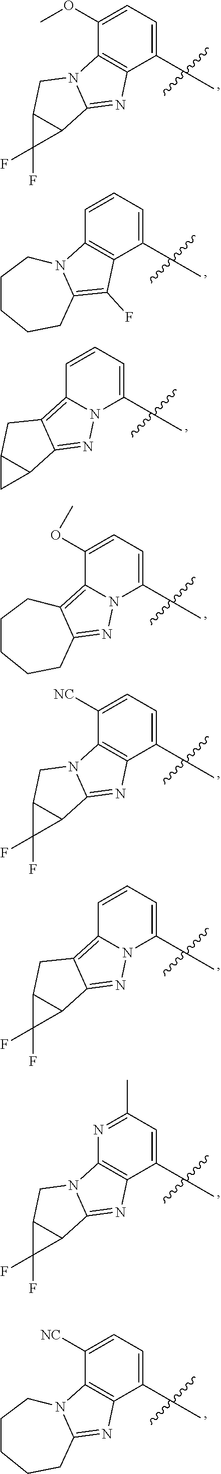

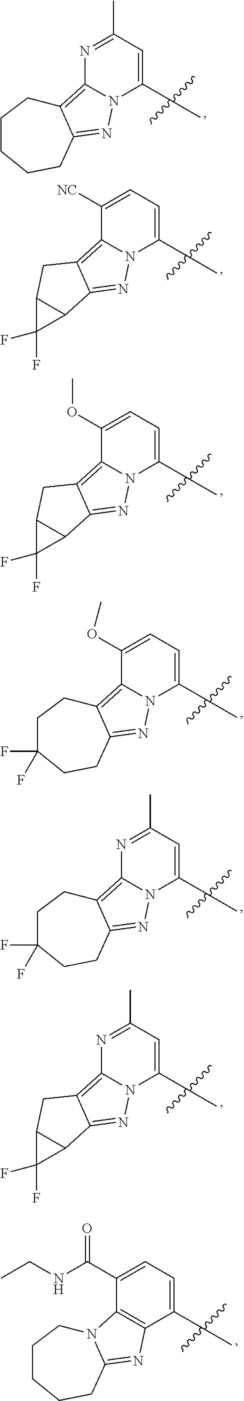

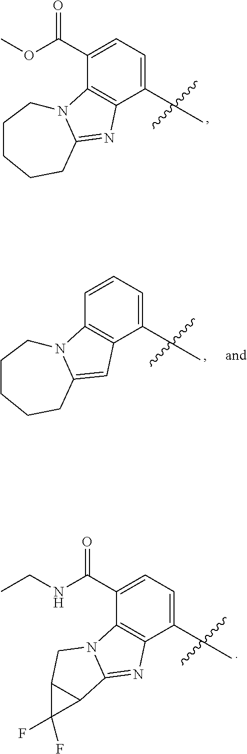 [SMILES: *c1ccc(C(=O)OC)c2c1nc1n2CCCCC1 ; *c1cccc2c1cc1n2CCCCC1 ; *c1ccc(C(=O)NCC)c2c1nc1n2CC2C1C2(F)F]

22. The compound of formula (II) or the pharmaceutically acceptable salt thereof according to claim 20, wherein the compound of formula (II) or the pharmaceutically acceptable salt thereof is a compound of formula (III) or a pharmaceutically acceptable salt thereof: 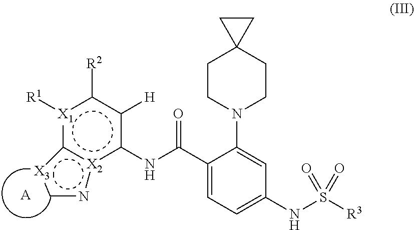
wherein:
A is a monocyclic or bicyclic ring selected from 4-10 membered carbon ring and 4-10 membered heterocycle, wherein the 4-10 membered carbon ring or 4-10 membered heterocycle is optionally substituted with one or more R^{b}, and each R^{b} is independently selected from halogen, OH, C₁-C₆ alkyl, O(C₁-C₄ alkyl), NH₂, NH(C₁-C₄ alkyl), and N(C₁-C₄ alkyl)₂, wherein the C₁-C₆ alkyl is optionally substituted with one or more groups independently selected from halogen, cyano, OH, NH₂, and O(C₁-C₄ alkyl);
X₁, X₂, and X₃ are each independently C or N;
R¹ is absent, H, C₁-C₆ alkyl, cyano, C₁-C₆ alkoxy, (CH₂)ᵣ—C(O)R^{d}, halogen, (CH₂)ᵣ—C₃-C₁₀ cycloalkyl, (CH₂)ᵣ—C₅-C₁₀ cycloalkenyl, (CH₂)ᵣ-4-10 membered heterocyclyl, (CH₂)ᵣ—C₆-C₁₀ aryl, or (CH₂)ᵣ-5-10 membered heteroaryl, wherein r is 0, 1, 2, or 3, and the C₁-C₆ alkyl, C₁-C₆ alkoxy, C₃-C₁₀ cycloalkyl, C₅-C₁₀ cycloalkenyl, 4-10 membered heterocyclyl, C₆-C₁₀ aryl, or 5-10 membered heteroaryl is optionally substituted with one or more groups independently selected from halogen, CN, OH, NH₂, C₁-C₆ alkyl, C₃-C₆ cycloalkyl, 4-7 membered heterocyclyl, O(C₁-C₄ alkyl), NH(C₁-C₄ alkyl), and N(C₁-C₄ alkyl)₂;
R² is H, C₁-C₆ alkyl, cyano, C₁-C₆ alkoxy, C(O)R^{d}, halogen, C₁-C₆ haloalkyl, or C₁-C₆ haloalkoxy;
R^{d} is selected from OH, C₁-C₆ alkyl, O(C₁-C₄ alkyl), NH₂, NH(C₁-C₄ alkyl), and N(C₁-C₄ alkyl)₂, and when one of R¹ and R² is H, then the other is not H, cyano, or halogen; and
R³ is selected from C₁-C₆ alkyl optionally substituted with one or more halogen or OH.
